(19) United States
(12) Patent Application Publication
(10) Pub. No.: US 2014/0243302 A1
(43) Pub. Date: Aug. 28, 2014

(54) BRIDGED BICYCLIC COMPOUNDS FOR THE TREATMENT OF BACTERIAL INFECTIONS

(71) Applicants: Yasumichi Fukuda, Shimotsuga-gun (JP); David E. Kaelin, JR., East Brunswick, NJ (US); Sheo B. Singh, Edison, NJ (US)

(72) Inventors: Yasumichi Fukuda, Shimotsuga-gun (JP); David E. Kaelin, JR., East Brunswick, NJ (US); Sheo B. Singh, Edison, NJ (US)

(73) Assignees: MERCK SHARP & DOHME CORPORATION, Rahway, NJ (US); KYORIN PHARMACEUTICAL CO., LTD., Tokyo (JP)

(21) Appl. No.: 14/126,225

(22) PCT Filed: Jun. 26, 2012
(86) PCT No.: PCT/US2012/044267

§ 371 Date: Mar. 3, 2014

Related U.S. Application Data
(60) Provisional application No. 61/501,692, filed on Jun. 27, 2011.

(51) Int. Cl. A61K 31/542; A61K 31/5377; A61K 31/498; A61K 31/4375; A61K 45/06; C07D 513/04; C07D 498/04; C07D 471/04; C07D 491/056; A61K 31/5383; C07D 515/04

(52) U.S. Cl. CPC A61K 31/542; A61K 31/5383; A61K 31/5377; A61K 31/498; A61K 31/4375; C07D 515/04; C07D 513/04; C07D 498/04; C07D 471/04; C07D 491/056; A61K 45/06
USPC 514/192; 514/210.02; 514/222.8; 514/224.2; 514/230.5; 514/234.5; 514/249; 514/275; 514/300; 514/302; 544/2; 544/48; 544/105; 544/122; 544/127; 544/331; 544/354; 546/115; 546/122

(57) ABSTRACT

Novel bridged bicyclic compounds are disclosed herein, along with their pharmaceutically acceptable salts, hydrates and prodrugs. Also disclosed are compositions comprising such compounds, methods of preparing such compounds and methods of using such compounds as antibacterial agents. The disclosed compounds, their pharmaceutically acceptable salts, hydrates and prodrugs, as well as compositions comprising such compounds, salts, hydrates and prodrugs, are useful for treating bacterial infections and associated diseases and conditions.

### CROSS REFERENCE TO RELATED APPLICATIONS

This application claims the benefit of and priority to U.S. provisional patent application No. 61/501,692 filed Jun. 27, 2011, which is incorporated herein by reference in its entirety as if set forth fully below.

### JOINT RESEARCH AGREEMENT

The present invention was made as a result of activities undertaken within the scope of joint research agreements between Merck & Co., Inc. and Kyorin Pharmaceutical Co., and between Merck & Co., Inc. and WuXi AppTec.

### FIELD OF THE INVENTION

The present invention relates to novel bridged bicyclic compounds (including pharmaceutically acceptable salts, hydrates and prodrugs thereof), compositions containing such compounds, synthesis of such compounds, and use of such compounds as antibacterial agents. The novel compounds of this disclosure and compositions comprising such compounds are useful for treating bacterial infections and associated diseases and conditions.

### BACKGROUND OF THE INVENTION

Bacterial infection is a major healthcare problem, and the incidence of hospital-acquired bacterial diseases continues to rise, particularly with drug-resistant strains. See Chu et al., 1996, J. Med. Chem. 39:3853-3874. As a result of drug resistance, many bacterial infections are either difficult to treat with today's antibiotics or even untreatable. This problem has become especially serious with the development of multiple drug resistance in certain strains of bacteria, such as Staphylococcus aureus, Streptococcus pneumoniae, Mycobacterium tuberculosis, Enterococcus sp. and Pseudomonas sp. The appearance of vancomycin resistant Enterococcus has been particularly alarming because vancomycin was formerly the only effective antibiotic for treating this infection, and had been considered for many infections to be the drug of “last resort”.

Hospitals, in particular, serve as centers for the formation and transmission of drug-resistant organisms. Infections occurring in hospitals, known as nosocomial infections, are becoming an increasingly serious problem. Of the two million Americans infected in hospitals each year, more than half of these infections resist at least one antibiotic. The Center for Disease Control reported that in 1992, over 13,000 hospital patients died of bacterial infections that were resistant to antibiotic treatment. See Lewis, “The Rise of Antibiotic-Resistant Infections”, FDA Consumer, Vol. 29, September 1995. The rate of infections continue to rise; as reported in 2007, over 18,000 patients died as a result of Methicillin-resistant S. aureus infections. See Klevens et al., 2007, J. Am. Med. Assoc. 298:1763-1771.

As bacterial resistance to antibiotics has become an important public health problem, there is a continuing need to develop newer and more potent antibiotics. More particularly, there is a need for antibiotics that represent a new class of compounds not previously used to treat bacterial infection. Such compounds would be particularly useful in treating nosocomial infections in hospitals where the formation and transmission of resistant bacteria are becoming increasingly prevalent.

### SUMMARY OF THE INVENTION

The present invention relates to bridged bicyclic compounds. These compounds, or pharmaceutically acceptable salts thereof, are useful in the treatment of bacterial infections caused by one or more of various pathogens including, but not limited to, Staphylococcus aureus. In particular, the present invention includes a compound of Formula I:

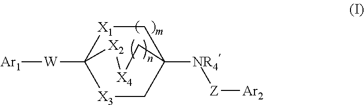

wherein:

X₁, X₂, and X₃ are independently CR₁R₂, O, S, S═O, SO₂ or NR₀;

X₄ is CR₁R₂, O, S, S═O, SO₂, NR₀, or is absent;

with the provisos that if X₂ is O, S, S═O, SO₂ or NR₀, then X₄ is CR₁R₂, if X₄ is O, S, S═O, SO₂ or NR₀, then X₂ is CR₁R₂, and no more than two of X₁, X₂, X₃ and X₄ are O, S, S═O, SO₂ or NR₀;

m is 1, 2, or 3;

n is 0, 1, or 2;

W is C(═O), —CR₁R₂—, —CH═CH—, —C≡C—, —CR₁R₂—CR₁′R₂′—, —O—CR₁R₂—, —NR₄—CR₁R₂—, or a group of the following structure:

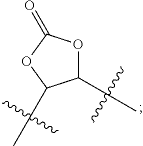 [SMILES: *C1OC(=O)OC1*]

R₀ is H, (C₁₋₆)alkyl, acyl or sulfonyl;

each R₁, R₂, R₁′, and R₂′ is independently H, (C₁₋₆)alkyl, (C₁₋₆)hydroxyalkyl, —CO₂R₃, —CONR₄R₅, halogen, OR₃, CF₃, aryl, heteroaryl or NHR₄;

with the proviso that R₁ is not OR₃ or NHR₄ when R₂ is OR₃ or NHR₄, and R₁′ is not OR₃ or NHR₄ when R₂′ is OR₃ or NHR₄;

wherein R₁ and R₂, or R₁′ and R₂′ on the same carbon together may form ═O or ═NOR₄;

R₃ is H, (C₁₋₆)alkyl, hydroxy(C₁₋₆)alkyl, or CF₃;

R₄, R_{4′} and R₅ are independently H, (C₁₋₆)alkyl, or CO₂R₃;

Z is CH₂, C(═O), CH₂—CH═CH, or SO₂;

Ar₁ is a group having one of the following structures:

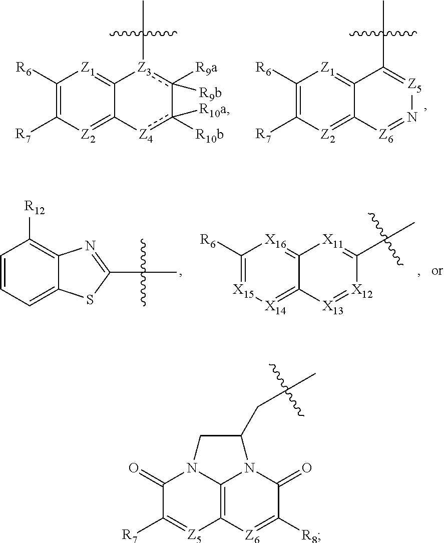

Z₁ is CR₁ₐ or N;

Z₂, Z₅ and Z₆ are independently CR_{1b}, or N;

Z₃ is C or N;

wherein Z₃ is not N if the [symbol] bond to which it is attached is a double bond;

Z₄ is CR₁₁ₐR_{11b}, N, CR₁₁ₐ, NR₁₁ₐ, or O;

wherein Z₄ is not O, NR₁₁ₐ or CR₁₁ₐR_{11b} if the [symbol] bond to which it is attached is a double bond;

X₁₁, X₁₃, X₁₄ and X₁₆ are independently N or CR₁ₐ;

wherein at least one of X₁₁, X₁₃, X₁₄ and X₁₆ is N;

X₁₂ is CH, C—(C₁₋₆)alkyl, C—(C₁₋₆)alkoxy, C-halo, or C—COOH;

X₁₅ is CH, C—(C₁₋₆)alkyl or C-halo;

R₆ is H; OH; NR₁₃R₁₄; (C₁₋₆)alkyl; C(O)OR₁₃; halo; CF₃; cyano; allyloxy; —R₁₅COOR₁₄; —OR₁₅COOR₁₄; (C₁₋₆)alkoxy, (C₃₋₆)cycloalkoxy, (C₃₋₆)heterocycleoxy, (C₃₋₆)cycloalkylalkoxy, or (C₃₋₆)heterocycloalkoxy which are optionally substituted with NR₁₃R₁₄, OH, CF₃, COOR₁₄, cyano, oxo, (C₁₋₆)alkyl or (C₁₋₆)alkoxy; S(O)₂R₁₃ optionally substituted with a (C₁₋₆)alkyl; or

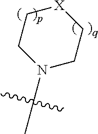

wherein X is CR_{1c}, O or S;

each p and q is 0, 1, or 2, with the proviso that if X is O or S, both p and q cannot be 0;

each R₇ and R₈ is independently H, halo, OH, (C₁₋₆)alkoxy, NR₁₃R₁₄, CF₃, or cyano;

R₉ₐ is H, halo, OH, (C₁₋₆)alkoxy, NH₂, or cyano; R_{9b} is absent; and the [symbol] bond attached to Z₃ is a double bond; or

R₉ₐ and R_{9b} together form oxo; and the [symbol] bond attached to Z₃ is a single bond;

R₁₀ₐ is H or (C₁₋₆)alkyl; R_{10b} is absent; and the [symbol] bond attached to Z₄ is a double bond; or

R₁₀ₐ and R_{10b} together form oxo; and the [symbol] bond attached to Z₄ is a single bond;

R₁₁ₐ is H or (C₁₋₆)alkyl; and R_{11b} is absent; and the [symbol] bond attached to Z₄ is a double bond or Z₄ is NR₁₁ₐ; or

R₁₁ₐ and R_{11b} together form oxo; and the [symbol] bond attached to Z₄ is a single bond;

or R₁₀ₐ and R₁₁ₐ together with the atoms to which they are attached form a 5-membered saturated, unsaturated or aromatic ring having 0 to 3 N and optionally substituted with a (C₁₋₆)alkyl, wherein R_{10b} and R_{11b} are H or absent, depending on valence;

each R₁₂, R₁₃ and R₁₄ is independently H or (C₁₋₆)alkyl;

each R₁₅ is independently (C₁-C₆)alkylene or (C₂-C₆)alkenylene with the proviso that when R₆ is —OR₁₅COOR₁₄, R₁₅ is not C₂alkenylene;

R₁ₐ is H, OH, (C₁₋₆)alkoxy, cyano, or halo;

R_{1b} is H, (C₁₋₆)alkyl, (C₁₋₆)alkoxy, halo, cyano, or C(O)OR₁₃;

R_{1c} is H, halo or (C₁₋₆)alkyl;

Ar₂ is

(i) C₃-C₆-cycloalkyl, optionally substituted with —OH, halo, cyano, NR₁₃R₁₄ or (C₁₋₆)alkyl;

(ii) aryl, wherein aryl is phenyl or naphthyl optionally substituted with 1 to 3 substituents selected from OH, halo, (C₁₋₆)alkoxy, halo(C₁₋₆)alkoxy and (C₁₋₆)alkyl;

(iii) a heterocyclyl, wherein the heterocyclyl is a 5- to 6-membered non-aromatic or aromatic ring having 1 or 2 heteroatoms selected from N, O or S optionally substituted with 1 to 3 substitutents selected from OH, halo, cyano, (C₁₋₆)alkoxy, (C₁₋₆)alkyl, NR₁₃R₁₄ and a 5- to 6-membered aromatic or non-aromatic ring having 1 or 2 heteroatoms selected from N, O or S; wherein (C₁₋₆)alkoxy or (C₁₋₆)alkyl optionally substituted with 1 or 2 halo; or

(iv) a group having one of the following structures:

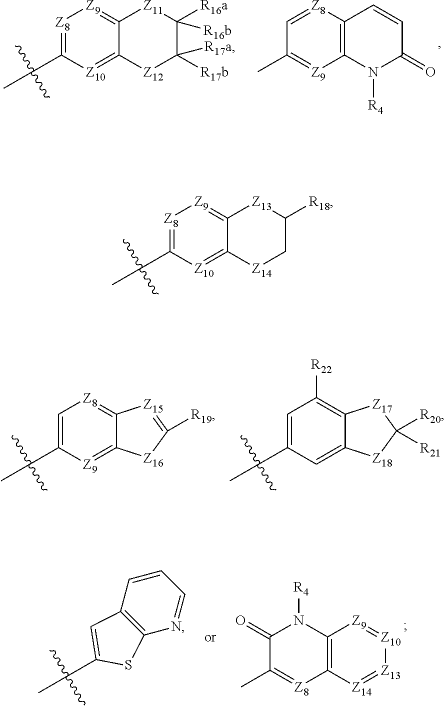

each Z₈, Z₉ and Z₁₀ is independently CR₁ₐ or N;

Z₁₁ and Z₁₂ are each independently CR₁ₐR_{1b}, NR₄, O, or S;

Z₁₃ and Z₁₄ are each independently CR₁ₐ or N;

Z₁₅ is CR₁ₐ or N;

Z₁₆ is CR₁ₐR_{1b} or NH;

each Z₁₇ and Z₁₈ is independently NR₄ or O;

each R₁₆ₐ and R_{16b} is independently H or CH₃;

or R₁₆ₐ and R_{16b} together form oxo;

each R₁₇ₐ and R_{17b} is H;

or R₁₇ₐ and R_{17b} together form oxo or ═NOR₃;

R₁₈ is H or (C₁₋₆)alkoxy;

R₁₉ is H or halo;

each R₂₀, R₂₁ and R₂₂ is independently H or halo;

or a pharmaceutically acceptable salt thereof.

In an embodiment, a compound of Formula (Ib) is provided:

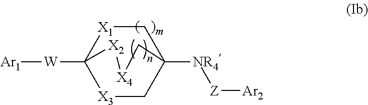

wherein:

X₁, X₂, and X₃ are independently CR₁R₂, O, S, S═O, SO₂ or NR₀;

X₄ is CR₁R₂, O, S, S═O, SO₂, NR₀, or is absent;

with the provisos that if X₂ is O, S, S═O, SO₂ or NR₀, then X₄ is CR₁R₂, if X₄ is O, S, S═O, SO₂ or NR₀, then X₂ is CR₁R₂, and no more than two of X₁, X₂, X₃ and X₄ are O, S, S═O, SO₂ or NR₀;

m is 1, 2, or 3;

n is 0, 1, or 2;

W is C(═O), —CR₁R₂—, —CH═CH—, —C≡—, —CR₁R₂—CR₁′R₂′—, —O—CR₁R₂—, —O—CR₁R₂—CR₁′R₂′—,

—NR₄—CR₁R₂—, or a group of the following structure:

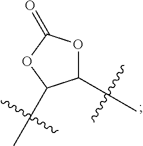 [SMILES: *C1OC(=O)OC1*]

R₀ is H, (C₁₋₆)alkyl, acyl or sulfonyl;

each R1, R2, R1′, and R2′ is independently H, (C₁₋₆)alkyl, (C₁₋₆)hydroxyalkyl, —CO₂R₃, —CONR₄R₅, halogen, OR₃, CF₃, aryl, heteroaryl or NHR₄;

with the proviso that R₁ is not OR₃ or NHR₄ when R₂ is OR₃ or NHR₄, and R₁′ is not OR₃ or NHR₄ when R₂′ is OR₃ or NHR₄;

wherein R₁ and R₂, or R₁′ and R₂′ on the same carbon together may form ═O or ═NOR₄;

R₃ is H, (C₁₋₆)alkyl, hydroxy(C₁₋₆)alkyl, or CF₃;

R₄, R₄′ and R₅ are independently H, (C₁₋₆)alkyl, or CO₂R₃;

Z is CH₂, C(═O), CH₂—CH═CH, CH₂—CH₂—O, or SO₂;

Ar₁ is a group having one of the following structures:

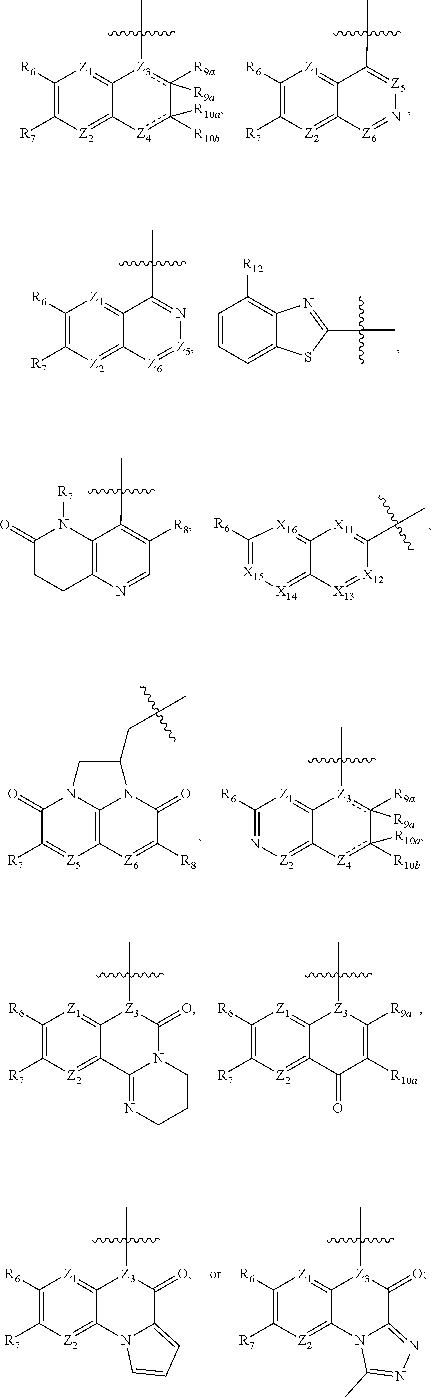

Z₁ is CR₁ₐ or N;

Z₂, Z₅ and Z₆ are independently CR_{1b}, or N;

Z₃ is C or N;

wherein Z₃ is not N if the [symbol] bond to which it is attached is a double bond;

Z₄ is CR₁₁ₐR_{11b}, N, CR₁₁ₐ, NR₁₁ₐ, or O;

wherein Z₄ is not O, NR₁₁ₐ or CR₁₁ₐR_{11b} if the [symbol] bond to which it is attached is a double bond;

X₁₁, X₁₃, X₁₄ and X₁₆ are independently N or CR₁ₐ;

wherein at least one of X₁₁, X₁₃, X₁₄ and X₁₆ is N;

X₁₂ is CH, C—(C₁₋₆)alkyl, C—(C₁₋₆)alkoxy, C-halo, or C—COOH;

X₁₅ is CH, C—(C₁₋₆)alkyl or C-halo;

R₆ is H; OH; NR₁₃R₁₄; (C₁₋₆)alkyl; C(O)OR₁₃; halo; CF₃; cyano; allyloxy; —R₁₅COOR₁₄; —OR₁₅COOR₁₄; —OR₁₅CONR₁₃R₁₄; (C₁₋₆)alkoxy, (C₃₋₆)cycloalkoxy, (C₃₋₆)heterocycleoxy, (C₃₋₁₀)cycloalkylalkoxy, or (C₃₋₁₀)heterocycloalkoxy which are optionally substituted with 1 to 3 substituents selected from NR₁₃R₁₄, CONR₁₃R₁₄, OH, halo, CF₃, COOR₁₄, cyano, oxo, (C₁₋₆)alkyl, or (C₁₋₆)alkoxy; S(O)₂R₁₃ optionally substituted with a (C₁₋₆)alkyl; or

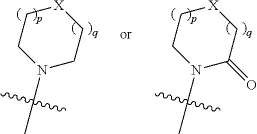

wherein X is CR_{1c}, O, S or SO₂;

each p and q is 0, 1, or 2, with the proviso that if X is O or S, both p and q cannot be 0;

each R₇ and R₈ is independently H, halo, OH, (C₁₋₆)alkoxy, NR₁₃R₁₄, CF₃, or cyano;

R₉ₐ is H, halo, OH, (C₁₋₆)alkoxy, NH₂, or cyano; R_{9b} is absent; and the [symbol] bond attached to Z₃ is a double bond; or

R₉ₐ and R_{9b} together form oxo; and the [symbol] bond attached to Z₃ is a single bond;

R₁₀ₐ is H or (C₁₋₆)alkyl; R_{10b} is absent; and the [symbol] bond attached to Z₄ is a double bond; or

R₁₀ₐ and R_{10b} together form oxo; and the [symbol] bond attached to Z₄ is a single bond;

R₁₁ₐ is H or (C₁₋₆)alkyl; and R_{11b} is absent; and the [symbol] bond attached to Z₄ is a double bond or Z₄ is NR₁₁ₐ; or

R₁₁ₐ and R_{11b} together form oxo; and the [symbol] bond attached to Z₄ is a single bond;

or R₁₀ₐ and R₁₁ₐ together with the atoms to which they are attached form a 5-membered saturated, unsaturated or aromatic ring having 0 to 3 N and optionally substituted with a (C₁₋₆)alkyl, wherein R_{10b} and R_{11b} are H or absent, depending on valence;

each R₁₂, R₁₃ and R₁₄ is independently H, (C₁₋₆)alkyl, or (C₁₋₆)hydroxyalkyl;

each R₁₅ is independently (C₁-C₆)alkylene or (C₂-C₆)alkenylene with the proviso that when R₆ is —OR₁₅COOR₁₄, R₁₅ is not C₂alkenylene;

R₁ₐ is H, OH, (C₁₋₆)alkoxy, cyano, or halo;

R_{1b} is H, (C₁₋₆)alkyl, (C₁₋₆)alkenyl, (C₁₋₆)alkoxy, halo, cyano, or C(O)OR₁₃;

R_{1c} is H, OH, halo or (C₁₋₆)alkyl;

Ar₂ is

(i) C₃-C₆-cycloalkyl, optionally substituted with —OH, halo, cyano, NR₁₃R₁₄ or (C₁₋₆)alkyl;

(ii) aryl, wherein aryl is phenyl or naphthyl optionally substituted with 1 to 3 substituents selected from OH, halo, (C₁₋₆)alkoxy, halo(C₁₋₆)alkoxy and (C₁₋₆)alkyl;

(iii) a heterocyclyl, wherein the heterocyclyl is a 5- to 6-membered non-aromatic or aromatic ring having 1 or 2 heteroatoms selected from N, O or S optionally substituted with 1 to 3 substitutents selected from OH, halo, cyano, (C₁₋₆)alkoxy, (C₁₋₆)alkyl, NR₁₃R₁₄ and a 5- to 6-membered aromatic or non-aromatic ring having 1 or 2 heteroatoms selected from N, O or S; wherein (C₁₋₆)alkoxy or (C₁₋₆)alkyl are optionally substituted with 1 or 2 halo; or

(iv) a group having one of the following structures:

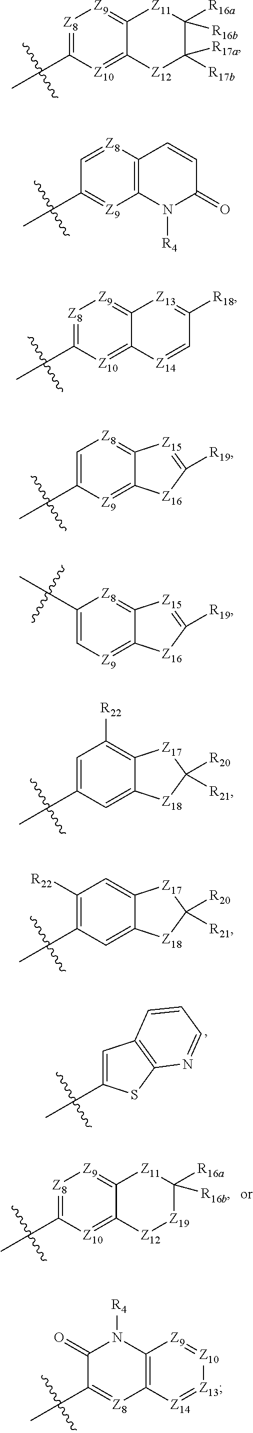

each Z₈, Z₉ and Z₁₀ is independently CR₁ₐ, CR_{1b} or N;

Z₁₁ and Z₁₂ are each independently CR₁ₐR_{1b}, NR₄, O, SO₂ or S;

Z₁₃ and Z₁₄ are each independently CR₁ₐ or N;

Z₁₅ is CR₁ₐ or N;

Z₁₆ is CR₁ₐR_{1b} or NH;

each Z₁₇ and Z₁₈ is independently NR₄ or O;

Z₁₉ is SO₂;

each R₁₆ₐ and R_{16b} is independently H or CH₃;

or R₁₆ₐ and R_{16b} together form oxo;

each R₁₇ₐ and R_{17b} is H;

or R₁₇ₐ and R_{17b} together form oxo or ═NOR₃;

R₁₈ is H or (C₁₋₆)alkoxy;

R₁₉ is H or halo;

each R₂₀, R₂₁ and R₂₂ is independently H or halo;

or R₂₀ and R₂₁ together form oxo;

or a pharmaceutically acceptable salt thereof.

These compounds are potent antibacterial agents useful against pathogens associated with bacterial infections.

Additional aspects of the invention relate to compositions comprising the compounds of the invention, optionally in the presence of a second therapeutic agent. In addition, aspects of the invention relate to methods of preparing a compound of the invention, to methods of preparing compositions of the invention, to methods of treating bacterial infection in patients using a compound of the invention, and to methods of controlling bacterial infection in patients using a compound of the invention.

Other embodiments, aspects and features of the present invention are either further described in or will be apparent from the ensuing description, examples and appended claims.

### DETAILED DESCRIPTION OF THE INVENTION

The present invention relates to compounds of Formula (I) and pharmaceutically acceptable salts thereof, as defined above and a first embodiment of the invention. Different embodiments further describing Formula (I) variables are described below.

In a second embodiment of the invention, the present invention relates to compounds of Formula (Ia) and pharmaceutically acceptable salts thereof

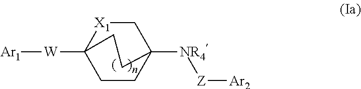

wherein:
X₁ is CH₂, O, or NR₀;
n is 0 or 1;
W is C(═O), —CH═CH—, —C≡C—, —CR₁R₂—CR₁R₂—, —CH₂—CR₁R₂—, —CR₁R₂—CH₂, —O—CR₁R₂—,
—NHR₄—CR₁R₂—, or a group of the following structure:

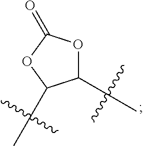 [SMILES: *C1OC(=O)OC1*]

each R₁ and R₂ is independently H, halo, (C₁₋₆)alkyl, OR₃, or NHR₄, wherein no more than one of R₁ or R₂ on the same carbon is OR₃ or NHR₄;
or R₁ and R₂ on the same carbon together form ═O or ═NOR₃;
R₃ is H or (C₁₋₆)alkyl;
Ar₁ is a group having one of the following structures:

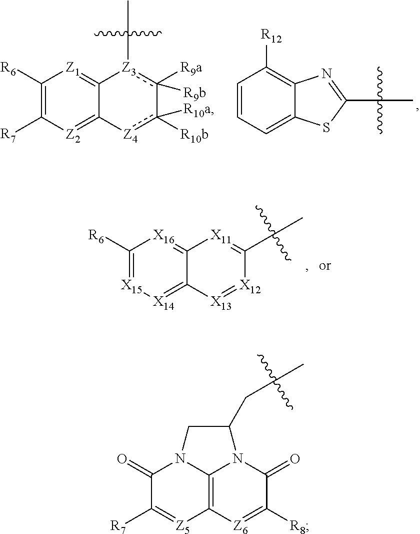

and all other variables as provided for in the first embodiment.

In an embodiment, a compound of Formula (Ib) is provided:

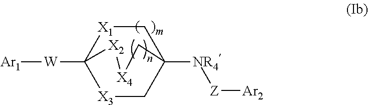

wherein:

X₁, X₂, and X₃ are independently CR₁R₂, O, S, S═O, SO₂ or NR₀;

X₄ is CR₁R₂, O, S, S═O, SO₂, NR₀, or is absent;

with the provisos that if X₂ is O, S, S═O, SO₂ or NR₀, then X₄ is CR₁R₂, if X₄ is O, S, S═O, SO₂ or NR₀, then X₂ is CR₁R₂, and no more than two of X₁, X₂, X₃ and X₄ are O, S, S═O, SO₂ or NR₀;

m is 1, 2, or 3;

n is 0, 1, or 2;

W is C(═O), —CR₁R₂—, —CH═CH—, —C≡C—, —CR₁R₂—CR₁′R₂′—, —O—CR₁R₂—, —O—CR₁R₂—CR₁′R₂′—,

—NR₄—CR₁R₂—, or a group of the following structure:

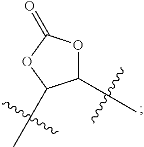 [SMILES: *C1OC(=O)OC1*]

R₀ is H, (C₁₋₆)alkyl, acyl or sulfonyl;

each R1, R2, R1′, and R2′ is independently H, (C₁₋₆)alkyl, (C₁₋₆)hydroxyalkyl, —CO₂R₃, —CONR₄R₅, halogen, OR₃, CF₃, aryl, heteroaryl or NHR₄;

with the proviso that R₁ is not OR₃ or NHR₄ when R₂ is OR₃ or NHR₄, and R₁′ is not OR₃ or NHR₄ when R₂′ is OR₃ or NHR₄;

wherein R₁ and R₂, or R₁′ and R₂′ on the same carbon together may form ═O or ═NOR₄;

R₃ is H, (C₁₋₆)alkyl, hydroxy(C₁₋₆)alkyl, or CF₃;

R₄, R₄′ and R₅ are independently H, (C₁₋₆)alkyl, or CO₂R₃;

Z is CH₂, C(═O), CH₂—CH═CH, CH₂—CH₂—O, or SO₂;

Ar₁ is a group having one of the following structures:

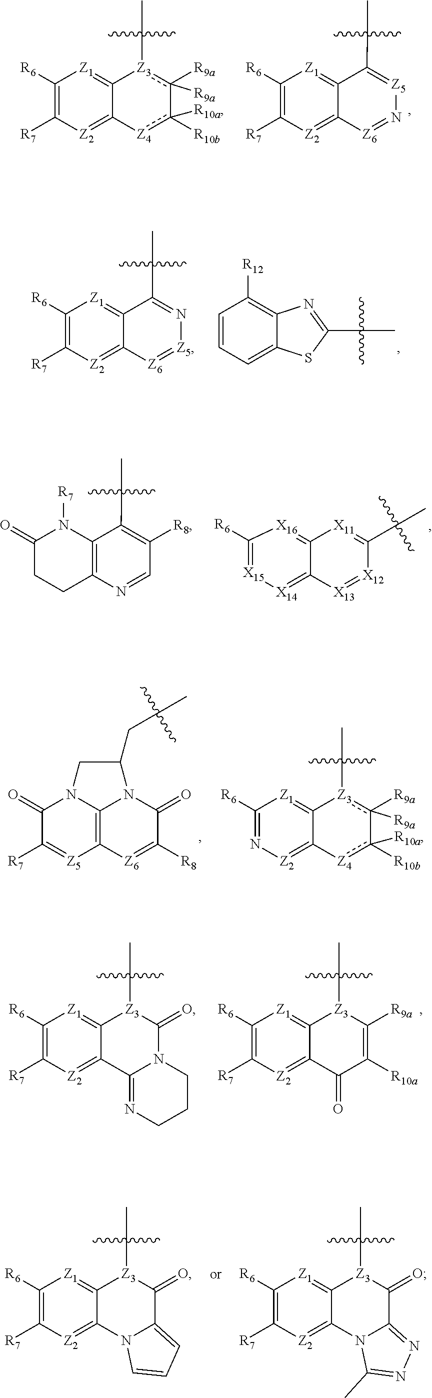

Z₁ is CR₁ₐ or N;

Z₂, Z₅ and Z₆ are independently CR_{1b}, or N;

Z₃ is C or N;

wherein Z₃ is not N if the [symbol] bond to which it is attached is a double bond;

Z₄ is CR₁₁ₐR_{11b}, N, CR₁₁ₐ, NR₁₁ₐ, or O;

wherein Z₄ is not O, NR₁₁ₐ or CR₁₁ₐR_{11b} if the [symbol] bond to which it is attached is a double bond;

X₁₁, X₁₃, X₁₄ and X₁₆ are independently N or CR₁ₐ;

wherein at least one of X₁₁, X₁₃, X₁₄ and X₁₆ is N;

X₁₂ is CH, C—(C₁₋₆)alkyl, C—(C₁₋₆)alkoxy, C-halo, or C—COOH;

X₁₅ is CH, C—(C₁₋₆)alkyl or C-halo;

R₆ is H; OH; NR₁₃R₁₄; (C₁₋₆)alkyl; C(O)OR₁₃; halo; CF₃; cyano; allyloxy; —R₁₅COOR₁₄; —OR₁₅COOR₁₄; —OR₁₅CONR₁₃R₁₄; (C₁₋₆)alkoxy, (C₃₋₆)cycloalkoxy, (C₃₋₆)heterocycleoxy, (C₃₋₁₀)cycloalkylalkoxy, or (C₃₋₁₀)heterocycloalkoxy which are optionally substituted with 1 to 3 substituents selected from NR₁₃R₁₄, CONR₁₃R₁₄, OH, halo, CF₃, COOR₁₄, cyano, oxo, (C₁₋₆)alkyl, or (C₁₋₆)alkoxy; S(O)₂R₁₃ optionally substituted with a (C₁₋₆)alkyl; or

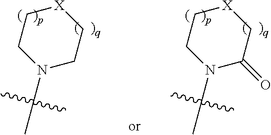

wherein X is CR_{1c}, O, S or SO₂;

each p and q is 0, 1, or 2, with the proviso that if X is O or S, both p and q cannot be 0;

each R₇ and R₈ is independently H, halo, OH, (C₁₋₆)alkoxy, NR₁₃R₁₄, CF₃, or cyano;

R₉ₐ is H, halo, OH, (C₁₋₆)alkoxy, NH₂, or cyano; R_{9b} is absent; and the [symbol] bond attached to Z₃ is a double bond; or

R₉ₐ and R_{9b} together form oxo; and the [symbol] bond attached to Z₃ is a single bond;

R₁₀ₐ is H or (C₁₋₆)alkyl; R_{10b} is absent; and the [symbol] bond attached to Z₄ is a double bond; or

R₁₀ₐ and R_{10b} together form oxo; and the [symbol] bond attached to Z₄ is a single bond;

R₁₁ₐ is H or (C₁₋₆)alkyl; and R_{11b} is absent; and the [symbol] bond attached to Z₄ is a double bond or Z₄ is NR₁₁ₐ; or

R₁₁ₐ and R_{11b} together form oxo; and the [symbol] bond attached to Z₄ is a single bond;

or R₁₀ₐ and R₁₁ₐ together with the atoms to which they are attached form a 5-membered saturated, unsaturated or aromatic ring having 0 to 3 N and optionally substituted with a (C₁₋₆)alkyl, wherein R_{10b} and R_{11b} are H or absent, depending on valence;

each R₁₂, R₁₃ and R₁₄ is independently H, (C₁₋₆)alkyl, or (C₁₋₆)hydroxyalkyl;

each R₁₅ is independently (C₁-C₆)alkylene or (C₂-C₆)alkenylene with the proviso that when R₆ is —OR₁₅COOR₁₄, R₁₅ is not C₂alkenylene;

R₁ₐ is H, OH, (C₁₋₆)alkoxy, cyano, or halo;

R_{1b} is H, (C₁₋₆)alkyl, (C₁₋₆)alkenyl, (C₁₋₆)alkoxy, halo, cyano, or C(O)OR₁₃;

R_{1c} is H, OH, halo or (C₁₋₆)alkyl;

Ar₂ is

(i) C₃-C₆-cycloalkyl, optionally substituted with —OH, halo, cyano, NR₁₃R₁₄ or (C₁₋₆)alkyl;

(ii) aryl, wherein aryl is phenyl or naphthyl optionally substituted with 1 to 3 substituents selected from OH, halo, (C₁₋₆)alkoxy, halo(C₁₋₆)alkoxy and (C₁₋₆)alkyl;

(iii) a heterocyclyl, wherein the heterocyclyl is a 5- to 6-membered non-aromatic or aromatic ring having 1 or 2 heteroatoms selected from N, O or S optionally substituted with 1 to 3 substitutents selected from OH, halo, cyano, (C₁₋₆)alkoxy, (C₁₋₆)alkyl, NR₁₃R₁₄ and a 5- to 6-membered aromatic or non-aromatic ring having 1 or 2 heteroatoms selected from N, O or S; wherein (C₁₋₆)alkoxy or (C₁₋₆)alkyl are optionally substituted with 1 or 2 halo; or

(iv) a group having one of the following structures:

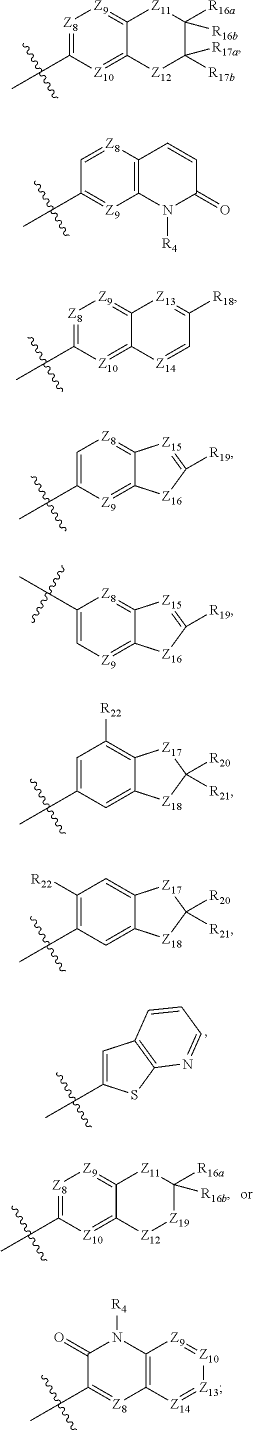

each Z₈, Z₉ and Z₁₀ is independently CR₁ₐ, CR_{1b} or N;

Z₁₁ and Z₁₂ are each independently CR₁ₐR_{1b}, NR₄, O, SO₂ or S;

Z₁₃ and Z₁₄ are each independently CR₁ₐ or N;

Z₁₅ is CR₁ₐ or N;

Z₁₆ is CR₁ₐR_{1b} or NH;

each Z₁₇ and Z₁₈ is independently NR₄ or O;

Z₁₉ is SO₂;

each R₁₆ₐ and R_{16b} is independently H or CH₃;

or R₁₆ₐ and R_{16b} together form oxo;

each R₁₇ₐ and R_{17b} is H;

or R₁₇ₐ and R_{17b} together form oxo or ═NOR₃;

R₁₈ is H or (C₁₋₆)alkoxy;

R₁₉ is H or halo;

each R₂₀, R₂₁ and R₂₂ is independently H or halo;

or R₂₀ and R₂₁ together form oxo;

or a pharmaceutically acceptable salt thereof.

In another embodiment, a compound of Formula (Ia), or a pharmaceutically acceptable salt thereof, is provided:

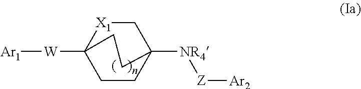

wherein:
X₁ is CH₂, O, or NR₀;
n is 0 or 1;
W is C(═O), —CH═CH—, —C≡C—, —CR₁R₂—CR₁R₂—, —O—CR₁R₂—CR₁R₂—, —CH₂—CR₁R₂—, —CR₁R₂—CH₂—, —O—CR₁R₂—, —NHR₄—CR₁R₂—, or a group of the following structure:

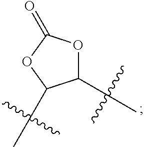 [SMILES: *C1OC(=O)OC1*]

each R₁ and R₂ is independently H, halo, (C₁₋₆)alkyl, OR₃, or NHR₄, wherein only one of R₁ or R₂ on the same carbon is OR₃ or NHR₄;
or R₁ and R₂ on the same carbon together form ═O or ═NOR₃;
R₃ is H or (C₁₋₆)alkyl;
Ar₁ is a group having one of the following structures:

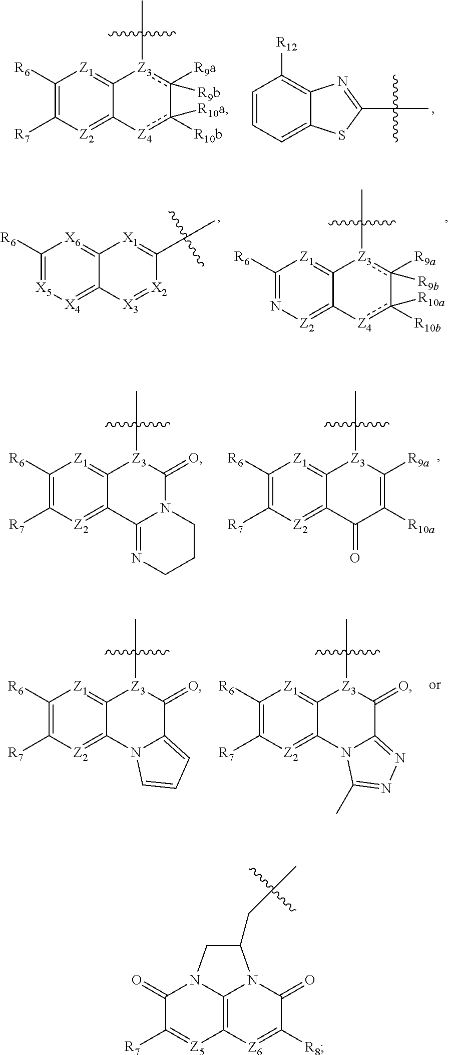

and all other variables are as defined in the context of Formula (Ib).

In a third embodiment of the invention, the present invention relates to compounds of Formula (I), (Ia), or (Ib) and pharmaceutically acceptable salts thereof, wherein
X₁ is CH₂ or O;
n is 1;
W is —CH═CH—, —C≡C—, —CR₁R₂—CR₁R₂—, —CH₂—CR₁R₂—, —CR₁R₂—CH₂— or —O—CH₂—;
each R₁ and R₂ is independently H, (C₁₋₆)alkyl or OH, wherein no more than one of R₁ or R₂ on the same carbon is OH;
R₄′ is H or (C₁₋₆)alkyl;
Z is CH₂ or CH₂—CH═CH;
Ar₁ is a group of the following structure:

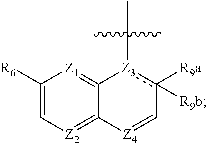

Z₄ is CR₁₁ₐ or N;
and no more than three of Z₁, Z₂, Z₃, and Z₄ are N;
R₆ is OH; (C₁₋₆)alkyl; halo; CF₃; cyano; (C₁₋₆)alkoxy, (C₃₋₆)cycloalkoxy, (C₃₋₆)heterocycleoxy, (C₃₋₆)cycloalkylalkoxy, or (C₃₋₆)heterocycloalkoxy which are optionally substituted with NR₁₃R₁₄, OH, CF₃, COOR₁₄, cyano, oxo or (C₁₋₆)alkoxy;
R₉ₐ is H, F, Cl, OH, (C₁₋₆)alkoxy, or cyano; R_{9b} is absent; and the [symbol] bond attached to Z₃ is a double bond; or
R₉ₐ and R_{9b} together form oxo; and the [symbol] bond attached to Z₃ is a single bond;
R₁₁ₐ is H or (C₁₋₆)alkyl;
R₁ₐ is H, halo or (C₁₋₆)alkoxy;
R_{1b} is H, (C₁₋₆)alkyl, halo, or (C₁₋₆)alkoxy;
Ar₂ is selected from
   aryl, wherein aryl is phenyl optionally substituted with 1 or 2 halo;
   or a group of the following structure:

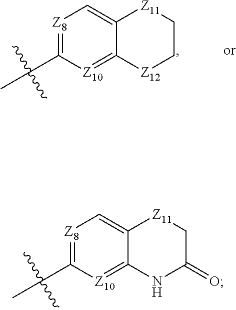

Z₁₀ is CH or N;
each Z₁₁ and Z₁₂ is CR₁ₐR_{1b}, N—(C₁₋₆)alkyl, O or S;
and the other variables are as provided for in the first or second embodiment.

In a fourth embodiment of the invention, the present invention relates to compounds of Formula (I), (Ia), or (Ib) and pharmaceutically acceptable salts thereof,
X₁ is CH₂ or O;
n is 1;
W is —CH₂—CH₂— or —CH₂—CHOH—;
Z is CH₂;
Ar₁ is a group having one of the following structures:

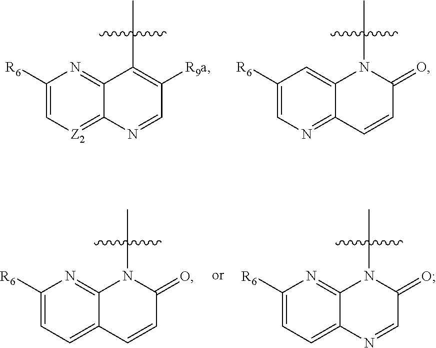

Z₂ is CR_{1b};
R₆ is (C₁₋₆)alkyl, halo, cyano, or (C₁₋₆)alkoxy, (C₃₋₆)cycloalkylalkoxy, or (C₃₋₆)heterocycloalkoxy which are optionally substituted with OH, COOR₁₄, cyano, or oxo;
R₉ₐ is F, Cl, OH, or cyano;
R_{1b} is H or (C₁₋₆)alkyl;
Ar₂ is a group having one of the following structures:

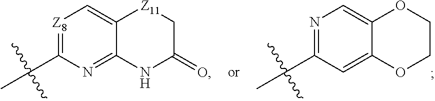

Z₈ is CR₁ₐ;
R₁ₐ is H, halo or (C₁₋₆)alkoxy;
Z₁₁ is O or S;

and the other variables are as provided for in any of the first through third embodiments.

In a fifth embodiment of the invention, the present invention relates to compounds of Formula (I), (Ia), or (Ib) and pharmaceutically acceptable salts thereof,

wherein
X₁ is CH₂ or O;
n is 1;
W is —CH₂—CH₂—, —CH═CH—, —C≡C—, —CH₂—CHOH—, —CHOH—CH₂—, —CH₂—C(CH₃)OH—, or —O—CH₂;
Z is CH₂ or —CH₂—CH═CH—;
An is a group having one of the following structures:

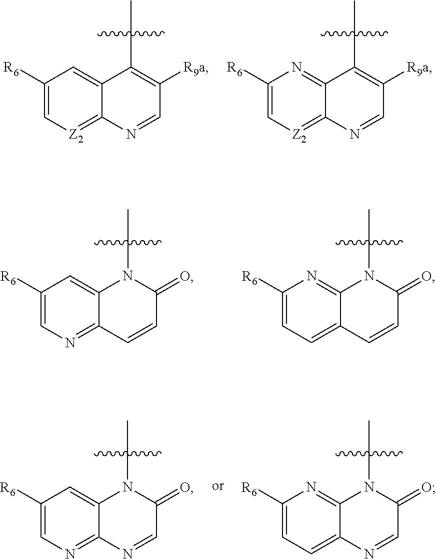

Z₂ is CR_{1b};
R₆ is (C₁₋₆)alkyl, halo, cyano, or (C₁₋₆)alkoxy, (C₃₋₆)cycloalkylalkoxy, or (C₃₋₆)heterocycloalkoxy which are optionally substituted with OH, COOR₁₄, cyano, or oxo;
R₉ₐ is H, F, Cl, OH, or cyano;
R_{1b} is H, F, Cl, or (C₁₋₆)alkyl;
Ar₂ is a group having one of the following structures:

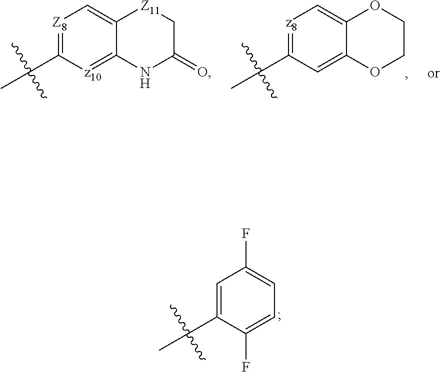

Z₈ and Z₁₀ are independently CR₁ₐ or N;
R₁ₐ is H, F, Cl, or (C₁₋₆)alkoxy;
Z₁₁ is O or S;

and the other variables are as provided for in any of the first through fourth embodiments.

In a sixth embodiment of the invention, the present invention relates to compounds of Formula (I), (Ia), or (Ib) and pharmaceutically acceptable salts thereof, wherein W is —CH₂—CHOH—, and the other variables are as provided for in any of the first through fifth embodiments.

In a seventh embodiment of the invention, the present invention relates to compounds of Formula (I), (Ia), or (Ib) and pharmaceutically acceptable salts thereof, wherein Ar₂ is

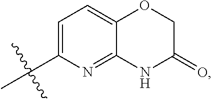 [SMILES: *c1ccc2c(n1)NC(=O)CO2]

and the other variables are as provided for in any of the first through sixth embodiments.

In an eighth embodiment of the invention, the present invention relates to compounds of Formula (I), (Ia), or (Ib) and pharmaceutically acceptable salts thereof, wherein

Ar₁ is

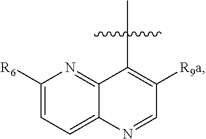

and the other variables are as provided for in any of the first through seventh embodiments.

Exemplary Ar₁ groups of this embodiment of the invention include but are not limited to the following:

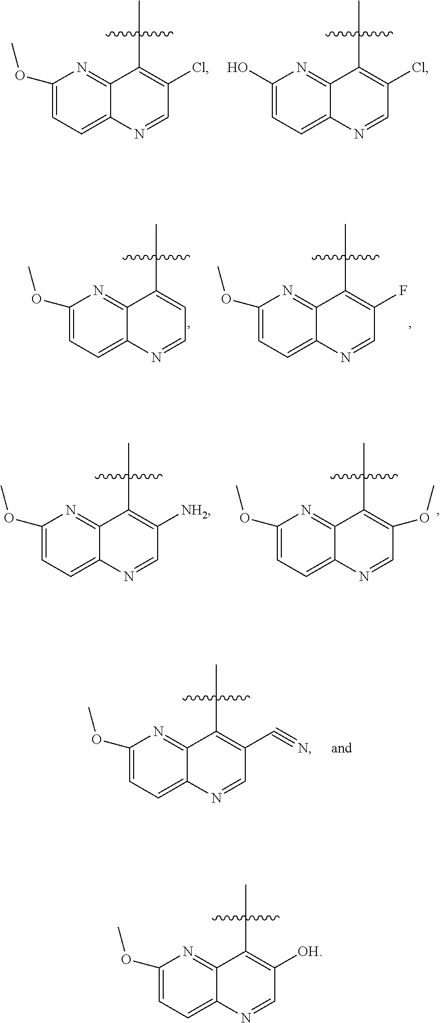

In a ninth embodiment of the invention, the present invention relates to compounds of Formula (I), (Ia), or (Ib) and pharmaceutically acceptable salts thereof, wherein X₁ is O and the other variables are as provided for in any of the first through eighth embodiments.

In an embodiment, each R₁, R₂, R₁′, and R₂′ is independently H, OH, (C₁₋₆)alkyl, or (C₁₋₆)hydroxyalkyl. In an embodiment, Ar₁ is

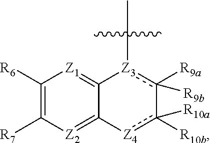

wherein Z₁-Z₄, R₆, R₇, R₉ₐ, R_{9b}, R₁₀ₐ and R_{10b} are as described in the context of formula I. In an embodiment, Ar₁ is

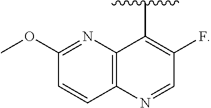 [SMILES: *c1c(F)cnc2ccc(OC)nc12]

In an embodiment, Ar₂ is

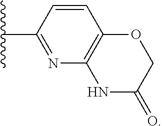 [SMILES: *c1ccc2c(n1)NC(=O)CO2]

In another embodiment of the invention, the compound of the invention is selected from the exemplary species depicted in Examples 1 through 190 shown below (including free base forms thereof and any pharmaceutically acceptable salts thereof). In an embodiment, the compound of the invention is selected from the exemplary species depicted in Examples 194 through 319 provided below (including free base forms thereof and any pharmaceutically acceptable salts thereof).

In certain embodiments, the compound of the invention is selected from the group consisting of:
(E)-6-((1-(2-(3-Fluoro-6-methoxy-1,5-naphthyridin-4-yl)vinyl)-2-oxabicyclo[2.2.2]octan-4-ylamino)methyl)-2H-pyrido[3,2-b][1,4]oxazin-3(4H)-one;
6-((1-(2-(3-Chloro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one;
N-((2,3-Dihydrobenzo[b][1,4]dioxin-6-yl)methyl)-1-(2-(3-fluoro-6-methoxy-1,5-naphthyridin-4-yl)ethyl)-2-oxabicyclo[2.2.2]octan-4-amine;
7-Chloro-6-(((1-(2-(3-fluoro-6-methoxy-1,5-naphthyridin-4-yl)ethyl)-2-oxabicyclo[2.2.2]octan-4-yl)amino)methyl)-2H-pyrido[3,2-b][1,4]oxazin-3(4H)-one;
6-((4-(2-(3-Fluoro-6-methoxy-1,5-naphthyridin-4-yl)ethyl)bicyclo[2.2.2]octan-1-ylamino)methyl)-2H-pyrido[3,2-b][1,4]oxazin-3(4H)-one;
6-((1-(2-(3-Fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one;
6-Methoxy-4-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-3-carbonitrile;
6-(((1-(1-Hydroxy-2-(7-methoxy-2-oxo-1,5-naphthyridin-1(2H)-yl)ethyl)-2-oxabicyclo[2.2.2]octan-4-yl)amino)methyl)-2H-pyrido[3,2-b][1,4]oxazin-3(4H)-one;
6-(((1-(2-(7-Methoxy-2-oxopyrido[2,3-b]pyrazin-1(2H)-yl)ethyl)-2-oxabicyclo[2.2.2]octan-4-yl)amino)methyl)-2H-pyrido[3,2-b][1,4]oxazin-3(4H)-one;
6-((1-(2-(7-Methoxy-2-oxo-1,8-naphthyridin-1(2H)-yl)ethyl)-2-oxabicyclo[2.2.2]octan-4-ylamino)methyl)-2H-pyrido[3,2-b][1,4]oxazin-3(4H)-one;
6-((1-(2-(3,8-difluoro-6-methoxyquinolin-4-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-methoxy-1,5-naphthyridin-4-yl)ethyl)-2-oxabicyclo[2.2.2]octan-4-ylamino)methyl)-2H-pyrido[3,2-b][1,4]thiazin-3(4H)-one;
7-Fluoro-6-(((1-(2-(3-fluoro-6-methoxy-1,5-naphthyridin-4-yl)ethyl)-2-oxabicyclo[2.2.2]octan-4-yl)amino)methyl)-2H-pyrido[3,2-b][1,4]oxazin-3(4H)-one;
(E)-6-((4-(2-(3-Chloro-6-methoxy-1,5-naphthyridin-4-yl)vinyl)bicyclo[2.2.2]octan-1-ylamino)methyl)-2H-pyrido[3,2-b][1,4]oxazin-3(4H)-one;
6-(((1-(2-(7-Methoxy-2-oxo-1,5-naphthyridin-1(2H)-yl)ethyl)-2-oxabicyclo[2.2.2]octan-4-yl)amino)methyl)-2H-pyrido[3,2-b][1,4]oxazin-3(4H)-one;
6-((1-(2-(3-Fluoro-6-methoxyquinolin-4-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-(3-hydroxypropoxy)-1,5-naphthyridin-4-yl)ethyl)-2-oxabicyclo[2.2.2]octan-4-ylamino)methyl)-2H-pyrido[3,2-b][1,4]oxazin-3(4H)-one;
N-[(2E)-3-(2,5-Difluorophenyl)prop-2-en-1-yl]-1-[2-(3-fluoro-6-methoxy-1,5-naphthyridin-4-yl)ethyl]-2-oxabicyclo[2.2.2]octan-4-amine;
6-((1-(2-(3-fluoro-6-methoxy-8-methyl-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(6-Methoxy-1,5-naphthyridin-4-yl)ethyl)-2-oxabicyclo[2.2.2]octan-4-ylamino)methyl)-2H-pyrido[3,2-b][1,4]oxazin-3(4H)-one;
6-((4-((3-Chloro-6-methoxy-1,5-naphthyridin-4-yloxy)methyl)bicyclo[2.2.2]octan-1-ylamino)methyl)-2H-pyrido[3,2-b][1,4]oxazin-3(4H)-one;
6-((1-(2-(7-Methoxy-2-oxo-1,8-naphthyridin-1(2H)-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-(2-hydroxyethoxy)-1,5-naphthyridin-4-yl)ethyl)-2-oxabicyclo[2.2.2]octan-4-ylamino)methyl)-2H-pyrido[3,2-b][1,4]oxazin-3(4H)-one;
6-((4-(2-(3-Chloro-6-methoxy-1,5-naphthyridin-4-yl)ethyl)bicyclo[2.2.2]octan-1-ylamino)methyl)-2H-pyrido[3,2-b][1,4]oxazin-3(4H)-one;
6-((1-(2-(6-Methoxy-1,5-naphthyridin-4-yl)ethyl)-2-oxabicyclo[2.2.2]octan-4-ylamino)methyl)-2H-pyrido[3,2-b][1,4]oxazin-3(4H)-one;
6-((4-((3-Chloro-6-methoxy-1,5-naphthyridin-4-yl)ethynyl)bicyclo[2.2.2]octan-1-ylamino)methyl)-2H-pyrido[3,2-b][1,4]oxazin-3(4H)-one;
6-((4-(2-(6-Methoxy-1,5-naphthyridin-4-yl)ethyl)bicyclo[2.2.2]octan-1-ylamino)methyl)-2H-pyrido[3,2-b][1,4]oxazin-3(4H)-one;
6-((4-(2-(3-Chloro-6-methoxy-1,5-naphthyridin-4-yl)-2-hydroxyethyl)bicyclo[2.2.2]octan-1-ylamino)methyl)-2H-pyrido[3,2-b][1,4]oxazin-3(4H)-one;
6-((4-(2-(3-Chloro-6-methoxy-1,5-naphthyridin-4-yl)-1-hydroxyethyl)bicyclo[2.2.2]octan-1-ylamino)methyl)-2H-pyrido[3,2-b][1,4]oxazin-3(4H)-one;
6-(((1-(1-Hydroxy-2-(6-methoxy-3-oxopyrido[2,3-b]pyrazin-4(3H)-yl)ethyl)-2-oxabicyclo[2.2.2]octan-4-yl)amino)methyl)-2H-pyrido[3,2-b][1,4]oxazin-3(4H)-one;
7-((1-(2-(3-Fluoro-6-methoxy-1,5-naphthyridin-4-yl)ethyl)-2-oxabicyclo[2.2.2]octan-4-ylamino)methyl)-1H-pyrido[3,4-b][1,4]oxazin-2(3H)-one;
7-Fluoro-6-((1-(2-(3-fluoro-6-methoxy-1,5-naphthyridin-4-yl)-1-hydroxyethyl)-2-oxabicyclo[2.2.2]octan-4-ylamino)methyl)-2H-pyrido[3,2-b][1,4]oxazin-3(4H)-one;
6-((4-(2-(3-Chloro-6-methoxy-1,5-naphthyridin-4-yl)-1-hydroxyethyl)bicyclo[2.2.2]octan-1-ylamino)methyl)-2H-pyrido[3,2-b][1,4]oxazin-3(4H)-one;
6-(((1-(1-Hydroxy-2-(7-methoxy-2-oxo-1,8-naphthyridin-1(2H)-yl)ethyl)-2-oxabicyclo[2.2.2]octan-4-yl)amino)methyl)-2H-pyrido[3,2-b][1,4]oxazin-3(4H)-one;
6-(((1-(2-(3-Hydroxy-6-methoxy-1,5-naphthyridin-4-yl)ethyl)-2-oxabicyclo[2.2.2]octan-4-yl)amino)methyl)-2H-pyrido[3,2-b][1,4]oxazin-3(4H)-one;
1-(4-((2,3-Dihydro-[1,4]dioxino[2,3-c]pyridin-7-yl)methylamino)-2-oxabicyclo[2.2.2]octan-1-yl)-2-(3-fluoro-6-methoxy-1,5-naphthyridin-4-yl)ethanol;
4-(2-(4-((2,3-Dihydro-[1,4]dioxino[2,3-c]pyridin-7-yl)methylamino)-2-oxabicyclo[2.2.2]octan-1-yl)ethyl)-6-methoxy-1,5-naphthyridine-3-carbonitrile;
6-((1-(2-(3-Fluoro-6-methoxy-1,5-naphthyridin-4-yl)-1-hydroxyethyl)-2-oxabicyclo[2.2.2]octan-4-ylamino)methyl)-2H-pyrido[3,2-b][1,4]oxazin-3(4H)-one;
6-(((1-(1-Hydroxy-2-(6-methoxy-3-oxopyrido[2,3-b]pyrazin-4(3H)-yl)ethyl)-2-oxabicyclo[2.2.2]octan-4-yl)amino)methyl)-2H-pyrido[3,2-b][1,4]oxazin-3(4H)-one;
7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-2-carbonitrile;
4-(2-(4-((2,3-Dihydro-[1,4]dioxino[2,3-c]pyridin-7-yl)methylamino)-2-oxabicyclo[2.2.2]octan-1-yl)ethyl)-6-methoxypyrido[3,2-b]pyrazin-3(4H)-one;
6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridine-3-carbonitrile;
6-(((1-(1-Hydroxy-2-(7-methoxy-2-oxo-1,8-naphthyridin-1(2H)-yl)ethyl)-2-oxabicyclo[2.2.2]octan-4-yl)amino)methyl)-2H-pyrido[3,2-b][1,4]oxazin-3(4H)-one;
6-((4-((3-Chloro-6-methoxy-1,5-naphthyridin-4-yloxy)methyl)bicyclo[2.2.2]octan-1-ylamino)methyl)-2H-pyrido[3,2-b][1,4]oxazin-3(4H)-one;
6-((1-((3-Fluoro-6-methoxy-1,5-naphthyridin-4-yloxy)methyl)-2-oxabicyclo[2.2.2]octan-4-ylamino)methyl)-2H-pyrido[3,2-b][1,4]oxazin-3(4H)-one;
((1-(1-(3-Fluoro-6-methoxy-1,5-naphthyridin-4-yl)-2-hydroxypropan-2-yl)-2-oxabicyclo[2.2.2]octan-4-ylamino)methyl)-2H-pyrido[3,2-b][1,4]oxazin-3(4H)-one;
6-((1-(2-(3-Fluoro-6-methoxy-1,5-naphthyridin-4-yl)-1-hydroxyethyl)-2-oxabicyclo[2.2.2]octan-4-ylamino)methyl)-2H-pyrido[3,2-b][1,4]thiazin-3(4H)-one;
6-((1-(2-(7-Methyl-2-oxo-1,8-naphthyridin-1(2H)-yl)ethyl)-2-oxabicyclo[2.2.2]octan-4-ylamino)methyl)-2H-pyrido[3,2-b][1,4]oxazin-3(4H)-one; and
6-((1-(2-(7-Fluoro-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one; and
   pharmaceutically acceptable salts thereof.

In certain embodiments, the compound of the invention is selected from the group consisting of:
1-(2-(3-Fluoro-6-methoxy-1,5-naphthyridin-4-yl)-3-hydroxypropyl)-N-((3-oxo-3,4-dihydro-2H-pyrido[3,2-b][1,4]oxazin-6-yl)methyl)-2-oxabicyclo[2.2.2]octan-4-aminium chloride;
sodium 2-(3-fluoro-6-methoxy-1,5-naphthyridin-4-yl)-3-(4-(((3-oxo-3,4-dihydro-2H-pyrido[3,2-b][1,4]oxazin-6-yl)methyl)amino)-2-oxabicyclo[2.2.2]octan-1-yl)propanoate;
7-Chloro-N-(4-(2-(3-fluoro-6-methoxy-1,5-naphthyridin-4-yl)ethyl)bicyclo[2.2.2]octan-1-yl)-3-oxo-3,4-dihydro-2H-pyrido[3,2-b][1,4]thiazine-6-carboxamide;
1-(2-(3-Fluoro-6-methoxy-1,5-naphthyridin-4-yl)ethyl)-N-((7-fluoro-8-methyl-3-oxo-3,4-dihydro-2H-pyrido[3,2-b][1,4]oxazin-6-yl)methyl)-2-oxabicyclo[2.2.2]octan-4-aminium chloride;
N-((7-Ethyl-8-methyl-3-oxo-3,4-dihydro-2H-pyrido[3,2-b][1,4]oxazin-6-yl)methyl)-1-(2-(3-fluoro-6-methoxy-1,5-naphthyridin-4-yl)ethyl)-2-oxabicyclo[2.2.2]octan-4-aminium chloride;
1-(2-(3-Fluoro-6-methoxy-1,5-naphthyridin-4-yl)ethyl)-N-((8-methyl-3-oxo-7-vinyl-3,4-dihydro-2H-pyrido[3,2-b][1,4]oxazin-6-yl)methyl)-2-oxabicyclo[2.2.2]octan-4-aminium chloride;
(R)—N-((2,3-Dihydro-[1,4]dioxino[2,3-c]pyridin-7-yl)methyl)-4-(2-(3-fluoro-6-methoxy-1,5-naphthyridin-4-yl)-1-hydroxyethyl)bicyclo[2.2.2]octan-1-aminium chloride;
(S)—N-((2,3-Dihydro-[1,4]dioxino[2,3-c]pyridin-7-yl)methyl)-4-(2-(3-fluoro-6-methoxy-1,5-naphthyridin-4-yl)-1-hydroxyethyl)bicyclo[2.2.2]octan-1-aminium chloride;
1-(2-(6-Cyano-3-oxo-2H-benzo[b][1,4]oxazin-4(3H)-yl)ethyl)-N-((3-oxo-3,4-dihydro-2H-pyrido[3,2-b][1,4]oxazin-6-yl)methyl)-2-oxabicyclo[2.2.2]octan-4-aminium chloride;
1-(2-(6-Bromo-3-oxo-2H-benzo[b][1,4]oxazin-4(3H)-yl)ethyl)-N-((3-oxo-3,4-dihydro-2H-pyrido[3,2-b][1,4]oxazin-6-yl)methyl)-2-oxabicyclo[2.2.2]octan-4-aminium chloride
(S)-1-(2-(3-Fluoro-6-methoxy-1,5-naphthyridin-4-yl)-1-hydroxyethyl)-N-((3-oxo-3,4-dihydro-2H-benzo[b][1,4]thiazin-6-yl)methyl)-2-oxabicyclo[2.2.2]octan-4-aminium chloride;
(S)—N-((1,1-Dioxido-3-oxo-3,4-dihydro-2H-benzo[b][1,4]thiazin-6-yl)methyl)-1-(2-(3-fluoro-6-methoxy-1,5-naphthyridin-4-yl)-1-hydroxyethyl)-2-oxabicyclo[2.2.2]octan-4-aminium chloride;
(S)-6-(((1-(2-(3-Fluoro-6-methoxy-1,5-naphthyridin-4-yl)-1-hydroxyethyl)-2-oxabicyclo[2.2.2]octan-4-yl)ammonio)methyl)-3,4-dihydro-2H-pyrido[3,2-b][1,4]oxazin-4-ium chloride;
6-(((1-(2-(3-Fluoro-6-methoxy-1,5-naphthyridin-4-yl)ethyl)-2-oxabicyclo[2.2.2]octan-4-yl)ammonio)methyl)-2,3-dihydro-1H-pyrido[2,3-b][1,4]oxazin-1-ium chloride;
6-(((1-(2-(3-Fluoro-6-methoxy-1,5-naphthyridin-4-yl)-2-hydroxyethyl)-2-thiabicyclo[2.2.2]octan-4-yl)amino)methyl)-2H-pyrido[3,2-b][1,4]oxazin-3(4H)-one;
1-(2-(3-Fluoro-6-(2-hydroxyethoxy)-1,5-naphthyridin-4-yl)-2-hydroxypropyl)-N-((3-oxo-3,4-dihydro-2H-pyrido[3,2-b][1,4]oxazin-6-yl)methyl)-2-oxabicyclo[2.2.2]octan-4-aminium chloride;
1-(2-(3-Fluoro-6-(3-hydroxypropoxy)-1,5-naphthyridin-4-yl)-2-hydroxyethyl)-N-((3-oxo-3,4-dihydro-2H-pyrido[3,2-b][1,4]oxazin-6-yl)methyl)-2-oxabicyclo[2.2.2]octan-4-aminium chloride;
4-(2-Hydroxy-2-(4-((3-oxo-3,4-dihydro-2H-pyrido[3,2-b][1,4]oxazin-6-yl)methylamino)-2-oxabicyclo[2.2.2]octan-1-yl)ethyl)-6-methoxy-1,5-naphthyridine-3-carbonitrile Hydrochloride;
6-(2-Hydroxyethoxy)-4-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-3-carbonitrile;
6-(3-Hydroxypropoxy)-4-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-3-carbonitrile;
6-((1-(2-(6-(((1S,3R,4S)-3,4-Dihydroxycyclopentyl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
8-(2-(4-((3-Oxo-3,4-dihydro-2H-pyrido[3,2-b][1,4]oxazin-6-yl)methylamino)-2-oxabicyclo[2.2.2]octan-1-yl)ethyl)-1,5-naphthyridine-2,7-dicarbonitrile;
6-((1-(1-Hydroxy-2-(7-methoxy-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one Hydrochloride;
6-((1-(2-(6-((1-Aminocyclopropyl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(7-Methoxy-4-oxoquinolin-1(4H)-yl)ethyl)-2-oxabicyclo[2.2.2]octan-4-ylamino)methyl)-2H-pyrido[3,2-b][1,4]oxazin-3(4H)-one;
1-(2-(4-((2,3-Dihydro-[1,4]dioxino[2,3-c]pyridin-7-yl)methylamino)-2-oxabicyclo[2.2.2]octan-1-yl)-2-hydroxyethyl)-7-methoxy-1,5-naphthyridin-2(1H)-one;
4-(2-(4-((2,3-Dihydro-[1,4]dioxino[2,3-c]pyridin-7-yl)methylamino)-2-oxabicyclo[2.2.2]octan-1-yl)-2-hydroxyethyl)-6-methoxypyrido[3,2-b]pyrazin-3(4H)-one;
6-((1-(2-(6-((3R,4S)-4-Aminotetrahydro furan-3-yloxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(6-((3S,4R)-4-Aminotetrahydrofuran-3-yloxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-(3-hydroxypropoxy)-1,5-naphthyridin-4-yl)-1-hydroxyethyl)-2-oxabicyclo[2.2.2]octan-4-ylamino)methyl)-2H-pyrido[3,2-b][1,4]oxazin-3(4H)-one Hydrochloride;
5-(2-(4-((2,3-Dihydro-[1,4]dioxino[2,3-c]pyridin-7-yl)methylamino)-2-oxabicyclo[2.2.2]octan-1-yl)-2-hydroxyethyl)-6-oxo-5,6-dihydro-1,5-naphthyridine-3-carbonitrile;
5-(2-Hydroxy-2-(4-((3-oxo-3,4-dihydro-2H-pyrido[3,2-b][1,4]oxazin-6-yl)methylamino)-2-oxabicyclo[2.2.2]octan-1-yl)ethyl)-6-oxo-5,6-dihydro-1,5-naphthyridine-3-carbonitrile;
5-(2-(4-((2,3-Dihydro-[1,4]dioxino[2,3-c]pyridin-7-yl)methylamino)-2-oxabicyclo[2.2.2]octan-1-yl)-2-hydroxyethyl)-6-oxo-5,6-dihydro-1,5-naphthyridine-3-carbonitrile;
5-(2-Hydroxy-2-(4-((3-oxo-3,4-dihydro-2H-pyrido[3,2-b][1,4]oxazin-6-yl)methylamino)-2-oxabicyclo[2.2.2]octan-1-yl)ethyl)-6-oxo-5,6-dihydro-1,5-naphthyridine-3-carbonitrile;
7-((1-(2-(3-Fluoro-6-methoxy-1,5-naphthyridin-4-yl)ethyl)-2-oxabicyclo[2.2.2]octan-4-ylamino)methyl)-1H-pyrido[2,3-e][1,3,4]oxathiazine-2,2-dioxide;
6-((1-(2-(6-(((2S,3R)-3-Amino-4-oxoazetidin-2-yl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(6-(((1r,3R,4S)-3,4-Dihydroxycyclopentyl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-((3-hydroxyoxetan-3-yl)methoxy)-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-(2-hydroxyethoxy)-1,5-naphthyridin-4-yl)-1-hydroxyethyl)-2-oxabicyclo[2.2.2]octan-4-ylamino)methyl)-2H-pyrido[3,2-b][1,4]oxazin-3(4H)-one Hydrochloride;
3-((1-(2-(3-Fluoro-6-methoxy-1,5-naphthyridin-4-yl)ethyl)-2-oxabicyclo[2.2.2]octan-4-ylamino)methyl)-1-methyl-1,8-naphthyridin-2(1H)-one Hydrochloride;
6-((1-(2-(3-Fluoro-6-((5-hydroxy-4-oxo-1,4-dihydropyridin-2-yl)methoxy)-1,5-naphthyridin-4-yl)ethyl)-2-oxabicyclo[2.2.2]octan-4-ylamino)methyl)-2H-pyrido[3,2-b][1,4]oxazin-3(4H)-one;
Methyl 2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylate;
6-((1-(2-(3-Fluoro-6-methoxy-1,5-naphthyridin-4-yl)-2-hydroxyethyl)-2-oxabicyclo[2.2.2]octan-4-ylamino)methyl)-2H-pyrido[3,2-b][1,4]oxazin-3(4H)-one;
3-((1-(2-(3-Fluoro-6-methoxy-1,5-naphthyridin-4-yl)ethyl)-2-oxabicyclo[2.2.2]octan-4-ylamino)methyl)-1-methyl-1,5-naphthyridin-2(1H)-one;
2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylic Acid;
6-((1-(2-(3-Fluoro-6-hydroxy-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-(2-(5-hydroxy-4-oxo-1,4-dihydropyridin-2-yl)ethoxy)-1,5-naphthyridin-4-yl)ethyl)-2-oxabicyclo[2.2.2]octan-4-ylamino)methyl)-2H-pyrido[3,2-b][1,4]oxazin-3(4H)-one;
7-((1-(2-(3-Fluoro-6-methoxy-1,5-naphthyridin-4-yl)ethyl)-2-oxabicyclo[2.2.2]octan-4-ylamino)methyl)-1H-pyrido[2,3-b][1,4]oxazin-2(3H)-one Hydrochloride;
6-((1-(2-(6-((3R,4S)-4-Aminotetrahydrofuran-3-yloxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
8-Chloro-3-((1-(2-(3-fluoro-6-methoxy-1,5-naphthyridin-4-yl)ethyl)-2-oxabicyclo[2.2.2]octan-4-ylamino)methyl)-1-methylquinolin-2(1H)-one;
(E)-6-Methoxy-4-(2-(4-((3-oxo-3,4-dihydro-2H-pyrido[3,2-b][1,4]oxazin-6-yl)methylamino)-2-oxabicyclo[2.2.2]octan-1-yl)vinyl)pyrido[3,2-c]pyridazine-3-carbonitrile;
6-((1-(1-Hydroxy-2-(7-(2-hydroxyethoxy)-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one;
6-Methoxy-4-(2-(4-((3-oxo-3,4-dihydro-2H-pyrido[3,2-b][1,4]oxazin-6-yl)methylamino)-2-oxabicyclo[2.2.2]octan-1-yl)ethyl)pyrido[3,2-c]pyridazine-3-carbonitrile;
6-((1-(1-Hydroxy-2-(7-(3-hydroxypropoxy)-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one;
6-((1-(2-(6-((3S,4S)-4-Amino-5-oxopyrrolidin-3-yloxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
methyl 2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylate;
2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylic Acid;
6-((1-(2-(6-(((2R,3R)-3-Aminooxetan-2-yl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
Ethyl 4-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanoate;
4-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanoic Acid;
6-((1-(2-(6-(((2S,3R)-3-Aminooxetan-2-yl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
7-((1-(2-(3-Fluoro-6-methoxy-1,5-naphthyridin-4-yl)ethyl)-2-oxabicyclo[2.2.2]octan-4-ylamino)methyl)-5-methylpyrido[3,2-b]pyrazin-6(5H)-one Hydrochloride;
6-((1-(2-(6-Methoxypyrido[3,2-d]pyrimidin-4-yl)ethyl)-2-oxabicyclo[2.2.2]octan-4-ylamino)methyl)-2H-pyrido[3,2-b][1,4]oxazin-3(4H)-one Hydrochloride;
6-((1-(2-(6-((2-Aminocyclopropyl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
4-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanenitrile;
ethyl 4-(7-Cyano-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanoate;
4-(7-Cyano-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanoic Acid;
6-((1-(2-(6-(((2S,3S)-3-Aminooxetan-2-yl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
4-(2-Hydroxy-2-(4-((3-oxo-3,4-dihydro-2H-pyrido[3,2-b][1,4]oxazin-6-yl)methylamino)-2-oxabicyclo[2.2.2]octan-1-yl)ethyl)-6-oxo-5,6,7,8-tetrahydro-1,5-naphthyridine-3-carbonitrile;
6-((1-(2-(7-(2-Hydroxyethoxy)-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one;
6-((1-(2-(7-(3-Hydroxypropoxy)-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one;
methyl 5-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)pentanoate;
2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylic Acid Hydrochloride;
5-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)pentanoic Acid;
methyl 7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-2-carboxylate;
7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-2-carboxylic Acid;
6-((1-(2-(6-(((2R,3S)-3-Aminooxetan-2-yl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
methyl 1-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylate;
1-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylic Acid;
methyl 2-((6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridin-3-yloxy)methyl)cyclopropanecarboxylate;
methyl 2-((6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridin-3-yloxy)methyl)cyclopropanecarboxylate;
4-(2-Hydroxy-2-(4-((3-oxo-3,4-dihydro-2H-pyrido[3,2-b][1,4]oxazin-6-yl)methylamino)-2-oxabicyclo[2.2.2]octan-1-yl)ethyl)-6-(2-hydroxyethoxy)-1,5-naphthyridine-3-carbonitrile;
4-(2-Hydroxy-2-(4-((3-oxo-3,4-dihydro-2H-pyrido[3,2-b][1,4]oxazin-6-yl)methylamino)-2-oxabicyclo[2.2.2]octan-1-yl)ethyl)-6-(3-hydroxypropoxy)-1,5-naphthyridine-3-carbonitrile;
methyl 2-((6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridin-3-yloxy)methyl)cyclopropanecarboxylate;
ethyl 2,2-Difluoro-4-(7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanoate;
2,2-Difluoro-4-(7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanamide;
methyl 2-((6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridin-3-yloxy)methyl)cyclopropanecarboxylate;
2,2-Difluoro-4-(7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanoic Acid;
1-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarbonitrile;
2-((6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridin-3-yloxy)methyl)cyclopropanecarboxylic Acid Hydrochloride;
6-((1-(2-(6-(Difluoromethoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
5,8-Difluoro-3-((1-(2-(3-fluoro-6-methoxy-1,5-naphthyridin-4-yl)ethyl)-2-oxabicyclo[2.2.2]octan-4-ylamino)methyl)-1-methylquinolin-2(1H)-one;
6-((1-(2-(3-Fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
2-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)-N-methylacetamide;
N-((5,8-Difluoro-2-methoxyquinolin-3-yl)methyl)-1-(2-(3-fluoro-6-methoxy-1,5-naphthyridin-4-yl)ethyl)-2-oxabicyclo[2.2.2]octan-4-amine;
N-(2-(2,5-Difluorophenoxy)ethyl)-1-(2-(3-fluoro-6-methoxy-1,5-naphthyridin-4-yl)ethyl)-2-oxabicyclo[2.2.2]octan-4-amine;
4-(7-Fluoro-8-((2-(4-((3-oxo-3,4-dihydro-2H-pyrido[3,2-b][1,4]oxazin-6-yl)methyl)amino)-2-oxabicyclo[2.2.2]octan-1-yl)ethyl)-1,5-naphthyridin-2-yl)thiomorpholine-1,1-dioxide;
2-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)-N,N-dimethylacetamide;
6-((1-(2-(3-Fluoro-6-methyl-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-(2-oxooxazolidin-3-yl)-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-(4-hydroxypiperidin-1-yl)-1,5-naphthyridin-4-yl)ethyl)-2-oxabicyclo[2.2.2]octan-4-ylamino)methyl)-2H-pyrido[3,2-b][1,4]oxazin-3(4H)-one;
(S)-6-((1-(2-(3-Fluoro-6-(3-hydroxypyrrolidin-1-yl)-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-(3-hydroxyazetidin-1-yl)-1,5-naphthyridin-4-yl)ethyl)-2-oxabicyclo[2.2.2]octan-4-ylamino)methyl)-2H-pyrido[3,2-b][1,4]oxazin-3(4H)-one;
(R)-6-((1-(2-(3-Fluoro-6-(3-hydroxypyrrolidin-1-yl)-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-(2-hydroxyethylamino)-1,5-naphthyridin-4-yl)ethyl)-2-oxabicyclo[2.2.2]octan-4-ylamino)methyl)-2H-pyrido[3,2-b][1,4]oxazin-3(4H)-one;
2-(8-(2-(4-((2,3-Dihydro-[1,4]dioxino[2,3-c]pyridin-7-yl)methylamino)-2-oxabicyclo[2.2.2]octan-1-yl)ethyl)-7-fluoro-1,5-naphthyridin-2-yloxy)-N-methylacetamide;
(E)-2-(8-(2-(4-(3-(2,5-Difluorophenyl)allylamino)-2-oxabicyclo[2.2.2]octan-1-yl)ethyl)-7-fluoro-1,5-naphthyridin-2-yloxy)-N-methylacetamide;
(E)-1-(2-(3-Fluoro-6-methoxy-1,5-naphthyridin-4-yl)ethyl)-N-(3-(pyridin-2-yl)allyl)-2-oxabicyclo[2.2.2]octan-4-amine;
   and pharmaceutically acceptable salts thereof.

Other embodiments of the present invention include the following (where reference to a compound of Formulas (I) or (Ib) encompasses the various embodiments and aspects described herein, as well as their pharmaceutically acceptable salts):

(a) A composition comprising a compound of Formula (I) or (Ib) and a carrier, adjuvant, or vehicle;

(b) A pharmaceutical composition comprising a compound of Formula (I) or

(Ib) and a pharmaceutically acceptable carrier, adjuvant, or vehicle;

(c) The pharmaceutical composition of (b), further comprising a second therapeutic agent;

(d) The pharmaceutical composition of (c), wherein the second therapeutic agent is a carbapenem, penicillin, cephalosporin or other β-lactam antibiotic;

(e) The pharmaceutical composition of (d), wherein the second therapeutic agent is imipenem or ertapenem;

(f) A pharmaceutical combination which is (1) a compound of Formula (I) or (Ib) and (2) a second therapeutic agent, wherein the compound of Formula (I) or (Ib) and the second therapeutic agent are each employed in an amount that renders the combination effective for treating bacterial infections;

(g) The combination of (f), wherein the second therapeutic agent is a carbapenems, penicillin, cephalosporin or other β-lactam antibiotic;

(h) The combination of (g), wherein the second therapeutic agent is imipenem or ertapenem;

(i) A method of treating a bacterial infections in a subject in need thereof comprising administering to the subject an effective amount of a compound of Formula (I) or (Ib);

(j) The method of (i), wherein the compound of Formula (I) or (Ib), is administered in combination, either sequentially or concurrently, with a second therapeutic agent effective against bacterial infections;

(k) The method of (j), wherein the second therapeutic agent is a carbapenem, penicillin, cephalosporin or other β-lactam antibiotic;

(l) The method of (k), wherein the second therapeutic agent is imipenem or ertapenem; and

(m) A method of treating bacterial infections in a subject in need thereof comprising administering to the subject a pharmaceutical composition of (b), (c), (d), or (e) or the combination of (f), (g) or (h).

The present invention also includes a compound of the present invention (i) for use in, (ii) for use as a medicine or medicament for, or (iii) for use in the preparation of a medicament for: treating bacterial infections. In these uses, the compounds of the present invention can optionally be employed in combination, either sequentially or concurrently, with one or more therapeutic agents effective against bacterial infections.

In the embodiments of the compound as provided herein, it is to be understood that each embodiment may be combined with one or more other embodiments, to the extent that such a combination provides a stable compound and is consistent with the description of the embodiments. It is further to be understood that the embodiments of compositions and methods provided as (a) through (m) herein are understood to include all embodiments of the compounds, including such embodiments as result from combinations of embodiments of the compound.

In addition, it is understood that, in the description of embodiments of the compounds as set forth herein, indicated substitutions are included only to the extent that the substitutents provide stable compounds consistent with the definition.

Additional embodiments of the invention include the pharmaceutical compositions, combinations and methods set forth in (a)-(m) herein and the uses set forth in the preceding paragraph, wherein the compound of the present invention employed therein is a compound of one of the embodiments or aspects of the compounds described herein. In all of these embodiments or aspects as well as those described hereinbelow, the compound may optionally be used in the form of a pharmaceutically acceptable salt or hydrate when appropriate.

In the compounds of generic Formula (I) or (Ib), the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I or (Ib). For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I or (Ib) can be prepared without undue experimentation by conventional techniques well known to those skilled in the art or by processes analogous to those described in the Schemes and Examples herein using appropriate isotopically-enriched reagents andor intermediates.

The present compounds (including pharmaceutical acceptable salt andor hydrate forms) have antimicrobial (e.g., antibacterial) activities and are useful for the treatment of bacterial infections. As used herein, unless otherwise indicated, the term “bacterial infection (s)” includes bacterial infections that occur in mammals as well as disorders related to bacterial infections that may be treated by administering antibiotics such as the compounds of the present invention. Such bacterial infections and disorders related to such infections include one or more of the following: pneumonia, otitis media, sinusitus, bronchitis, tonsillitis, and mastoiditis related to infection by Streptococcus pneumoniae, Haemophilus influenzae, Moraxella catarrhalis, Staphylococcus aureus, or Peptostreptococcus spp.; pharynigitis, rheumatic fever, and glomerulonephritis related to infection by Streptococcus pyogenes, Groups C and G streptococci, Clostridium diptheriae, or Actinobacillus haemolyticum; respiratory tract infections related to infection by Mycoplasma pneumoniae, Legionella pneumophila, Streptococcus pneumoniae, Haemophilus influenzae, or Chlamydia pneumoniae; uncomplicated skin and soft tissue infections, abscesses and osteomyelitis, and puerperal fever related to infection by Staphylococcus aureus, coagulase-positive staphylococci (i.e., S. epidermidis, S. hemolyticus, etc.), Streptococcus pyogenes, Streptococcus agalactiae, Streptococcal groups C-F (minute-colony streptococci), viridans streptococci, Corynebacterium minutissimum, Clostridium spp., or Bartonella henselae; uncomplicated acute urinary tract infections related to infection by Staphylococcus saprophyticus or Enterococcus spp.; urethritis and cervicitis; and sexually transmitted diseases related to infection by Chlamydia trachormatis, Haemophilus ducreyi, Treponema pallidum, Ureaplasma urealyticum, or Neisseria gonorrheae; toxin diseases related to infection by S. aureus (food poisoning and Toxic shock syndrome), or Groups A, S, and C streptococci; ulcers related to infection by Helicobacter pylori; systemic febrile syndromes related to infection by Borrelia recurrentis; Lyme disease related to infection by Borrelia burgdorferi; conjunctivitis, keratitis, and dacrocystitis related to infection by Chlamydia trachomatis, Neisseria gonorrhoeae, S. aureus, S. pneumoniae, S. pyogenes, H. influenza, or Listeria spp.; disseminated Mycobacterium avium complex (MAC) disease related to infection by Mycobacterium avium, or Mycobacterium intracellulare; gastroenteritis related to infection by Campylobacter jejuni; intestinal protozoa related to infection by Cryptosporidium spp. odontogenic infection related to infection by viridans streptococci; persistent cough related to infection by Bordetella pertussis; gas gangrene related to infection by Clostridium perfringens or Bacteroides spp.; and atherosclerosis related to infection by Helicobacter pylori or Chlamydia pneumoniae.

Bacterial infections and disorders related to such infections that may be treated or prevented in animals include one or more of the following: bovine respiratory disease related to infection by P. haem., P. multocida, Mycoplasma bovis, or Bordetella spp.; cow enteric disease related to infection by E. coli; dairy cow mastitis related to infection by S. aureus, Streptococcus uberis, Streptococcus agalactiae, Streptococcus dysgalactiae, Klebsiella spp., Corynebacterium spp., or Enterococcus spp.; swine respiratory disease related to infection by Actinobacillus pleurpneumoniae, Pasteurella multocida, or Mycoplasma spp.; swine enteric disease related to infection by E. coli, Lawsonia intracellularis, Salmonella, or Serpulina hyodyisinteriae; cow footrot related to infection by Fusobacterium spp.; cow metritis related to infection by E. coli; cow hairy warts related to infection by Fusobacterium necrophorum or Bacteroides nodosus; cow pink-eye related to infection by Moraxella bovis; urinary tract infection in dogs and cats related to infection by E. coli; skin and soft tissue infections in dogs and cats related to infection by S. epidermidis, S. interrmedius, coagulase neg. Staphylococcus or P. multocida; and dental or mouth infections in dogs and oats related to infection by Alcaligenes spp., Bacteroides spp., Clostridium spp., Enterobacter spp., Eubacterium, Peptostreptococcus, Porphfyromonas, or Prevotella.

In one embodiments, the bacterial infections and disorders related to such infections includes one or more of the following: Staphylococcus aureus Smith, Enterococcus faecium A2373, Streptococcus pneumoniae IID554, and Escherichia coli ATCC 25922.

Other bacterial infections and disorders related to such infections that may be treated or prevented in accord with the method of the present invention are referred to in J. P. Sanford et al., “The Sanford Guide To Antimicrobial Therapy,”26th Edition, (Antimicrobial Therapy, Inc., 1996).

Examples of carbapenems that may be co-administered with the compounds of the invention include, but are not limited to, imipenem, meropenem, biapenem, (4R,5S,6S)-3-[3S,5S)-5-(3-carboxyphenyl-carbamoyl)pyrrolidin-3-ylthio]-6-(1R)-1-hydroxyethyl]-4-methyl-7-oxo-1-azabicyclo[3.2.0]hept-2-ene-2-carboxylic acid (ertapenem), (1S,5R,6S)-2-(4-(2-(((carbamoylmethyl)-1,4-diazoniabicyclo[2.2.2]oct-1-yl)-ethyl (1,8-naphthosultam)methyl)-6-[1(R)-hydroxyethyl]-1-methylcarbapen-2-em-3-carboxylate chloride, BMS181139 ([4R-[4α,5β,6β(R*)]]-4-[2-[(aminoiminomethyl)amino]ethyl]-3-[(2-cyanoethyl)thio]-6-(1-hydroxyethyl)-7-oxo-1-azabicyclo[3.2.0]hept-2-ene-2-carboxylic acid), B02727 ([4R-3[3S*,5S*(R*)],4α,5β,6β(R*)]]-6-(1-hydroxyethyl)-3-[[5-[1-hydroxy-3-(methylamino)propyl]-3-pyrrolidinyl]thio]-4-methyl-7-oxo-1-azabicyclo[3.2.0]hept-2-ene-2-carboxylic acid monohydro chloride), E1010 ((1R,5S,6S)-6-[1(R)-hydroxymethyl]-2-[2(5)-[1(R)-hydroxy-1-[pyrrolidin-3(R)-yl]methyl]pyrrolidin-4(S)-ylsulfanyl]-1-methyl-1-carba-2-penem-3-carboxylic acid hydrochloride) and S4661 ((1R,5S,6S)-2-[(3S,5S)-5-(sulfamoylaminomethyl)pyrrolidin-3-yl]thio-6-[(1R-1-hydroxyethyl]-1-methylcarbapen-2-em-3-carboxylic acid), and (1S,5R,6S)-1-methyl-2-{7-[4-(aminocarbonylmethyl)-1,4-diazoniabicyclo(2.2.2)octan-lyl]-methyl-fluoren-9-on-3-yl}-6-(1R-hydroxyethyl)-carbapen-2-em-3 carboxylate chloride.

Examples of penicillins suitable for co-administration with the compounds according to the invention include benzylpenicillin, phenoxymethylpenicillin, carbenicillin, azidocillin, propicillin, ampicillin, amoxycillin, epicillin, ticarcillin, cyclacillin, pirbenicillin, azloccillin, mezlocillin, sulbenicillin, piperacillin, and other known penicillins. The penicillins may be used in the form of pro-drugs thereof; for example as in vivo hydrolysable esters, for example, the acetoxymethyl, pivaloyloxymethyl, α-ethoxycarbonyloxy-ethyl and phthalidyl esters of ampicillin, benzylpenicillin and amoxycillin; as aldehyde or ketone adducts of penicillins containing a 6-α-aminoacetamido side chain (for example hetacillin, metampicillin and analogous derivatives of amoxycillin); and as α-esters of carbenicillin and ticarcillin, for example the phenyl and indanyl α-esters.

Examples of cephalosporins that may be co-administered with the compounds according to the invention include, cefatrizine, cephaloridine, cephalothin, cefazolin, cephalexin, cephacetrile, cephapirin, cephamandole nafate, cephradine, 4-hydroxycephalexin, cephaloglycin, cefoperazone, cefsulodin, ceftazidime, cefuroxime, cefmetazole, cefotaxime, ceftriaxone, and other known cephalosporins, all of which may be used in the form of pro-drugs thereof.

Examples of β-lactam antibiotics other than penicillins and cephalosporins that may be co-administered with the compounds according to the invention include aztreonam, latamoxef (MOXALACTAM), and other known β-lactam antibiotics such as serine β-lactamase inhibitors including, but are not limited to, clavulanic acid, sulbactam or tazobactam.

When the compounds of Formula I or (Ib) are combined with a carbapenem antibiotic, a dehydropeptidase (DHP) inhibitor may also be combined. Many carbapenems are susceptible to attack by a renal enzyme known as DHP. This attack or degradation may reduce the efficacy of the carbapenem antibacterial agent Inhibitors of DHP and their use with carbapenems are disclosed in for example European Patent Application Publication No. EP 0007614. An exemplary DHP inhibitor is 7-(L-2-amino-2-carboxyethylthio)-2-(2,2-dimethylcyclopropanecarboxamide)-2-heptenoic acid or a useful salt thereof.

The term “acyl”, as used herein, refers to a carbonyl containing substituent represented by the formula —C(O)—R in which R is H, alkyl, a cycloalkyl, an aryl, a heterocycle, cycloalkyl- or aryl-substituted alkyl or heterocycle-substituted alkyl wherein the alkyl, alkoxy, cycloalkyl, aryl and heterocycle are as defined herein. Representative acyl groups include, but are not limited to, alkanoyl (e.g. acetyl), aroyl (e.g. benzoyl), and heteroaroyl.

The term “sulfonyl”, as used herein, refers to a substituent represented by the formula —S(O)₂—R in which R is H, alkyl, a cycloalkyl, an aryl, a heterocycle, cycloalkyl- or aryl-substituted alkyl or heterocycle-substituted alkyl wherein the alkyl, alkoxy, cycloalkyl, aryl and heterocycle are as defined herein.

The term “alkenyl”, as used herein, refers to a straight or branched-chain acyclic unsaturated hydrocarbon having a number of carbon atoms in the specified range and containing at least one double bond. Thus, for example, “C₂-C₃ alkenyl” refers to vinyl, (1Z)-1-propenyl, (1E)-1-propenyl, 2-propenyl, or isopropenyl.

The term “alkoxy”, as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl”, as used herein, refers to any linear or branched chain alkyl group having a number of carbon atoms in the specified range, for example 1-8, 1-6 or 1-4. Thus, for example, “C₁₋₆ alkyl” (or “C₁-C₆ alkyl”) refers to all of the hexyl alkyl and pentyl alkyl isomers as well as n-, iso-, sec- and t-butyl, n- and isopropyl, ethyl and methyl. As another example, “C₁₋₄ alkyl” refers to n-, iso-, sec- and t-butyl, n- and isopropyl, ethyl and methyl. C₁₋₆ alkyl and C₁₋₄ alkyl are examples of lower alkyls.

The term “aryl”, as used herein, refers to a mono- or bicyclic carbocyclic ring system having one or two aromatic rings. Exemplary aryls include, but are not limited to, phenyl, naphthyl, tetrahydronaphthyl, indanyl, indenyl and the like. Aryl groups (including bicyclic aryl groups) can be unsubstituted (unless otherwise indicated, such groups are unsubstituted) or substituted with one, two or three substituents independently selected from lower alkyl, substituted lower alkyl, haloalkyl, alkoxy, thioalkoxy, amino, alkylamino, dialkylamino, acylamino, cyano, hydroxy, halo, mercapto, nitro, carboxaldehyde, carboxy, alkoxycarbonyl and carboxamide.

The term “cycloalkylalkoxy” refers to a cycloalkyl group, as defined herein, appended to the parent molecular moiety through an alkoxy group, as defined herein. The cycloalkyl group may have one or more carbon atoms in common with the alkoxy group. A (C₃₋₆)cycloalkylalkoxy refers to a C₃₋₆ cycloalkyl group attached to an alkoxy group. Representative examples of cycloalkylalkoxy include 2-(1-ethylcyclopropyl)methoxy, 2-(1-propylcyclopropoxy), 2-(2-ethylcyclopropoxy), 2-(3-ethylcyclohexyl)methoxy, 2-(4-ethylcyclohexyl)methoxy, 2-(4-propylcyclohexyl)methoxy, 2-(2-(4-propylcyclohexyl)ethoxy), 2-(2-ethylcyclopentyl)methoxy, and 2-(2-propylcyclopentyloxy)pyridine.

The terms “cycloalkoxy” or “cycloalkyloxy” refers to a cycloalkyl group, as defined herein, appended to the parent molecular moiety through an oxygen atom. Representative examples of cycloalkyloxy include, but are not limited to, cyclopropyloxy, cyclobutyloxy, cyclopentyloxy, cyclohexyloxy, cycloheptyloxy, and cyclooctyloxy.

The term “cycloalkyl”, as used herein, refers to any cyclic ring of an alkane having a number of carbon atoms in the specified range. Thus, for example, “C₃₋₆ cycloalkyl” (or “C₃-C₆ cycloalkyl”) refers to cyclopropyl, cyclobutyl, cyclopentyl, and cyclohexyl.

The term “halogen” (or “halo”), as used herein, refers to fluorine, chlorine, bromine and iodine (alternatively referred to as fluoro, chloro, bromo, and iodo).

The term “heteroaryl”, as used herein, refers to a cyclic aromatic radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms. Exemplary heteroaryls include, but are not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like. Heteroaryl groups (including bicyclic heteroaryl groups) can be unsubstituted or substituted with one, two or three substituents independently selected from lower alkyl, substituted lower alkyl, haloalkyl, alkoxy, thioalkoxy, amino, alkylamino, dialkylamino, acylamino, cyano, hydroxy, halo, mercapto, nitro, carboxaldehyde, carboxy, alkoxycarbonyl and carboxamide.

The term “heterocycle” (and variations thereof such as “heterocyclic” or “heterocyclyl”), as used herein, broadly refers to (i) a stable 4- to 8-membered, saturated or unsaturated monocyclic ring, and the ring system contains one or more heteroatoms (e.g., from 1 to 6 heteroatoms, or from 1 to 4 heteroatoms) selected from N, O and S and a balance of carbon atoms (the monocyclic ring typically contains at least one carbon atom and the ring systems typically contain at least two carbon atoms); and wherein any one or more of the nitrogen and sulfur heteroatoms is optionally oxidized, and any one or more of the nitrogen heteroatoms is optionally quaternized. Unless otherwise specified, the heterocyclic ring may be attached at any heteroatom or carbon atom, provided that attachment results in the creation of a stable structure. Heterocycle groups (including bicyclic heterocycle groups) can be unsubstituted or substituted with one, two or three substituents independently selected from lower alkyl, substituted lower alkyl, haloalkyl, alkoxy, thioalkoxy, amino, alkylamino, dialkylamino, acylamino, cyano, hydroxy, halo, mercapto, nitro, carboxaldehyde, carboxy, alkoxycarbonyl and carboxamide. Unless otherwise specified, when the heterocyclic ring has substituents, it is understood that the substituents may be attached to any atom in the ring, whether a heteroatom or a carbon atom, provided that a stable chemical structure results.

The term “heterocycloalkoxy” means a heterocycle group, as defined herein, appended to the parent molecular moiety through an alkoxy group, as defined herein. The heterocycle group may have one or more carbon atoms in common with the alkoxy group. A (C₃₋₆)heterocycloalkoxy refers to a C₃₋₆ heterocycle group attached to an alkoxy group. Representative examples of heterocycloalkoxy include, but are not limited to, 2-(5-ethyltetrahydro-2H-pyran-2-yl)methoxy), 2-pyridin-3-ylethoxy, 3-quinolin-3-ylpropoxy, and 5-pyridin-4-ylpentyloxy.

The term “heterocycleoxy” means a heterocycle group, as defined herein, appended to the parent molecular moiety through an oxygen atom. Representative examples of heterocycleoxy include, but are not limited to, pyridin-3-yloxy and quinolin-3-yloxy.

The term “oxo”, as used herein, means ═O and as used herein, the term “imino” means ═NR₀, wherein R₀ is as previously defined.

The term “optionally substituted with 1 to 3 substituents,” as used herein, means optional substitution with 1, 2 or 3 substituents, where the 1, 2 or 3 substitutents may be the same or different, or two may be the same and one may be different. Where the substituents are selected from categories of substituents, the 1, 2 or 3 substitutents may be selected from the same or different categories, or two may be selected from the same category and one may be selected from a different category.

The term “or”, as used herein, denotes alternatives that may, where appropriate, be combined.

Unless expressly stated to the contrary, all ranges cited herein are inclusive. For example, a heterocyclic ring described as containing from “1 to 4 heteroatoms” means the ring can contain 1, 2, 3 or 4 heteroatoms. It is also to be understood that any range cited herein includes within its scope all of the sub-ranges within that range. Thus, for example, a heterocyclic ring described as containing from “1 to 4 heteroatoms” is intended to include as aspects thereof, heterocyclic rings containing 2 to 4 heteroatoms, 3 or 4 heteroatoms, 1 to 3 heteroatoms, 2 or 3 heteroatoms, 1 or 2 heteroatoms, 1 heteroatom, 2 heteroatoms, and so forth.

Any of the various cycloalkyl and heterocyclic/heteroaryl rings and ring systems defined herein may be attached to the rest of the compound at any ring atom (i.e., any carbon atom or any heteroatom) provided that a stable compound results. Suitable 5- or 6-membered heteroaromatic rings include, but are not limited to, pyridyl, pyrrolyl, pyrazinyl, pyrimidinyl, pyridazinyl, triazinyl, thienyl, furanyl, imidazolyl, pyrazolyl, triazolyl, tetrazolyl, oxazolyl, isooxazolyl, oxadiazolyl, oxatriazolyl, thiazolyl, isothiazolyl, and thiadiazolyl. Suitable 9- or 10-membered heteroaryl rings include, but are not limited to, quinolinyl, isoquinolinyl, indolyl, indazolyl, benzimidazolyl, benztriazoyl, imidazopyridinyl, triazolopyridinyl, and imidazopyrimidinyl. Suitable 4- to 6-membered heterocyclyls include, but are not limited to, azetidinyl, piperidinyl, morpholinyl, thiomorpholinyl, thiazolidinyl, isothiazolidinyl, oxazolidinyl, isoxazolidinyl, pyrrolidinyl, imidazolidinyl, piperazinyl, tetrahydrofuranyl, tetrahydrothienyl, pyrazolidinyl, hexahydropyrimidinyl, thiazinanyl, thiadiazinanyl, tetrahydropyranyl, tetrahydrothiopyranyl, and dioxanyl.

A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject). Reference to a compound also includes stable complexes of the compound such as a stable hydrate.

As a result of the selection of substituents and substituent patterns, certain of the compounds of the present invention can have asymmetric centers and can occur as mixtures of stereoisomers, or as individual diastereomers, or enantiomers. Unless otherwise indicated, all isomeric forms of these compounds, whether isolated or in mixtures, are within the scope of the present invention. Also included within the scope of the present invention are tautomeric forms of the present compounds as depicted.

When any variable occurs more than one time in any constituent or in Formula (I) or in any other formula depicting and describing compounds of the invention, its definition on each occurrence is independent of its definition at every other occurrence. Also, combinations of substituents andor variables are permissible only if such combinations result in stable compounds.

The terms “substituted” and “optionally substituted” include mono- and poly-substitution by a named substituent to the extent such single and multiple substitution (including multiple substitution at the same site) is chemically allowed. Hence, the terms specifically contemplate one or more substitutions. Unless expressly stated to the contrary, substitution by a named substituent is permitted on any atom in a ring (e.g., an aryl, a cycloalkyl, a heteroaryl, or a heterocyclyl) provided such ring substitution is chemically allowed and results in a stable compound.

Compounds of the present invention may be administered in the form of “pharmaceutically acceptable salts”, hydrates, esters, etc., as appropriate. Other salts may, however, be useful in the preparation of the compounds according to the invention or of their pharmaceutically acceptable salts. For example, when the compounds of the present invention contain a basic amine group, they may be conveniently isolated as trifluoroacetic acid salts (e.g. following HPLC purification). Conversion of the trifluoroacetic acid salts to other salts, including pharmaceutically acceptable salts, may be accomplished by a number of standard methods known in the art. For example, an appropriate ion exchange resin may be employed to generate the desired salt. Alternatively, conversion of a trifluoroacetic acid salt to the parent free amine may be accomplished by standard methods known in the art (e.g. neutralization with an appropriate inorganic base such as NaHCO₃). Other desired amine salts may then be prepared in a conventional manner by reacting the free base with a suitable organic or inorganic acid. Representative pharmaceutically acceptable quaternary ammonium salts include the following: hydrochloride, sulfate, phosphate, carbonate, acetate, tartrate, citrate, malate, succinate, lactate, stearate, fumarate, hippurate, maleate, gluconate, ascorbate, adipate, gluceptate, glutamate, glucoronate, propionate, benzoate, mesylate, tosylate, oleate, lactobionate, laurylsulfate, besylate, caprylate, isetionate, gentisate, malonate, napsylate, edisylate, pamoate, xinafoate, napadisylate, hydrobromide, nitrate, oxalate, cinnamate, mandelate, undecylenate, and camsylate. Many of the compounds of the invention carry an acidic carboxylic acid moiety, in which case suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

The present invention includes within its scope prodrugs of the compounds of this invention. In general, such prodrugs will be functional derivatives of the compounds of this invention which are readily convertible in vivo into the required compound. Thus, in the methods of treatment of the present invention, the term “administering” shall encompass the treatment of the various conditions described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs,” ed. H. Bundgaard, Elsevier, 1985, which is incorporated by reference herein in its entirety. Metabolites of these compounds include active species produced upon introduction of compounds of this invention into the biological milieu.

The term “administration” and variants thereof (e.g., “administering” a compound) in reference to a compound of the invention mean providing the compound or a prodrug of the compound to the subject in need of treatment. When a compound of the invention or a prodrug thereof is provided in combination with one or more other active agents (e.g., other antibacterial agents useful for treating bacterial infections), “administration” and its variants are each understood to include concurrent and sequential provision of the compound or prodrug and other agents.

As used herein, the term “composition” is intended to encompass a product comprising the specified ingredients, as well as any product which results, directly or indirectly, from combining the specified ingredients.

By “pharmaceutically acceptable,” it is meant that the ingredients of the pharmaceutical composition must be compatible with each other and not deleterious to the recipient thereof.

The term “subject” (alternatively referred to herein as “patient”) as used herein refers to an animal, preferably a mammal, most preferably a human, who has been the object of treatment, observation or experiment.

The term “effective amount” as used herein means that amount of active compound or pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. When the active compound (i.e., active ingredient) is administered as the salt, references to the amount of active ingredient are to the free acid or free base form of the compound.

For the purpose of treating bacterial infection, the compounds of the present invention, optionally in the form of a salt or a hydrate, can be administered by means that produces contact of the active agent with the agent's site of action. They can be administered by conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered by one or more of the following: orally, parenterally (including subcutaneous injections, intravenous, intramuscular, intrasternal injection or infusion techniques), by inhalation (e.g., nasal or buccal inhalation spray, aerosols from metered dose inhalator, and dry powder inhalator), by nebulizer, ocularly, topically, transdermally, or rectally, in the form of a unit dosage of a pharmaceutical composition containing an effective amount of the compound and conventional non-toxic pharmaceutically-acceptable carriers, adjuvants and vehicles. Liquid preparations suitable for oral administration (e.g., suspensions, syrups, elixirs and the like) can be prepared according to techniques known in the art and can employ the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions of the present invention and of ingredients suitable for use in said compositions is provided in Remington's Pharmaceutical Sciences, 20^{th} edition, edited by A. R. Gennaro, Mack Publishing Co., 2000.

The compounds of this invention can be administered, e.g., orally or intravenously, in a dosage range of, for example, 0.001 to 1000 mgkg of mammal (e.g., human) body weight per day in a single dose or in divided doses. An example of a dosage range is 0.01 to 500 mgkg body weight per day orally or intravenously in a single dose or in divided doses. Another example of a dosage range is 0.1 to 100 mgkg body weight per day orally or intravenously in single or divided doses. For oral administration, the compositions can be provided in the form of tablets or capsules containing, for example, 1.0 to 500 milligrams of the active ingredient, particularly 1, 5, 10, 15, 20, 25, 50, 75, 100, 150, 200, 250, 300, 400, and 500 milligrams of the active ingredient for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The present invention also includes processes for making compounds of Formula (I). The compounds of the present invention may be prepared according to the following reaction schemes and examples, using the appropriate intermediates and starting materials described in the Intermediates and Experimentals sections below, or modifications thereof.

In cases where Ar₁ contains an acidic methyl group Me-Ar₁ can be treated with an appropriate base, for example lithium diisopropylamide (LDA), and allowed to react with an aldehyde of the general structure I to give II, wherein W=—CH₂CHOH (Scheme 1). The nitrogen protecting group can be removed using, in the case of Boc, HCl or TFA to give III. Combination of III with an appropriate aldehyde using conditions capable of reductive amination (e.g. NaBH(OAc)₃) yields the final compound IV.

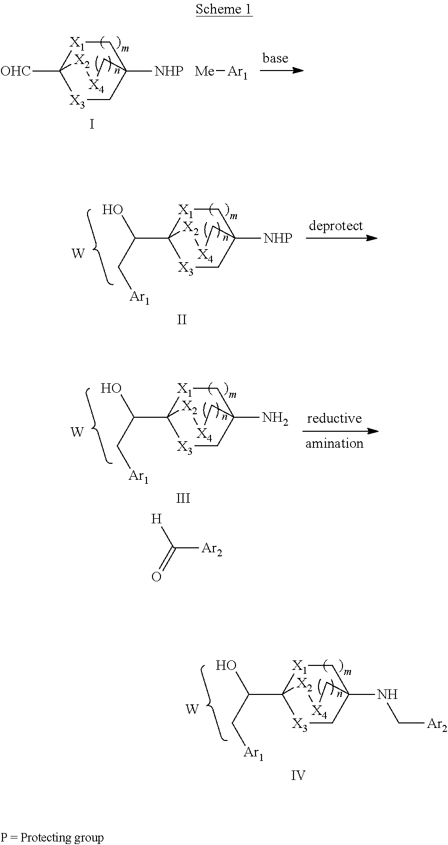

Alternatively, the hydroxyl group of compound II can be alkylated or acylated using conditions familiar to those skilled in the art to give V, which can be further transformed to desired products using the method described in Scheme 1 (Scheme 2).

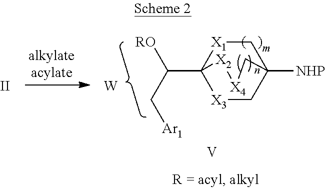

In another embodiment, an intermediate of the general structure III can be treated with either an alkyl or acyl chloride or an alkyl or aryl sulfonyl chloride in the presence of an appropriate base to give VI or VII, respectively (Scheme 3).

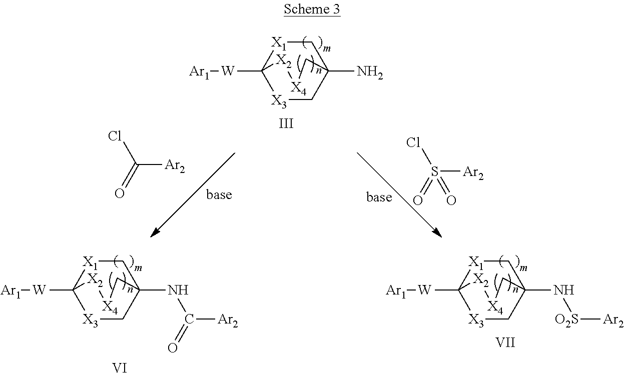

An alternate class of compounds can be prepared by reacting VIII with the appropriate aryl bromide in the presence of an appropriate palladium catalyst to give IX, which can be transformed into the final products by nitrogen deprotection followed by derivatization (Scheme 4).

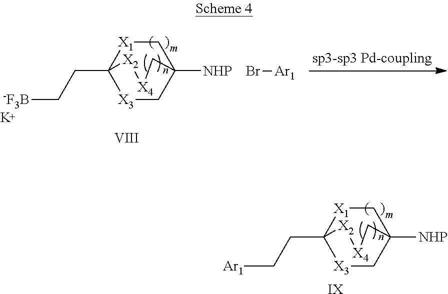

An additional class of compounds can be prepared by reacting X with the appropriate aryl bromide in the presence of an appropriate palladium catalyst to give XI, which can be transformed into the final products by nitrogen deprotection followed by derivatization (Scheme 5).

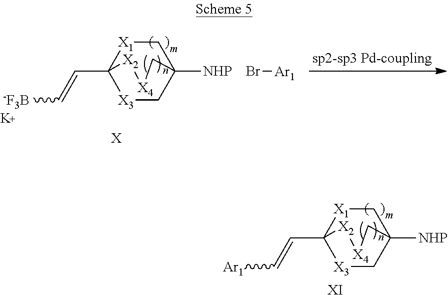

An additional class of compounds can be prepared by reacting XII with the appropriate aryl bromide in the presence of an appropriate palladium catalyst to give XIII, which can be transformed into the final products by nitrogen deprotection followed by derivatization (Scheme 6). Compounds of the structure XIII can be transformed to the corresponding trans olefin by catalytic hydrogenation to give XIV.

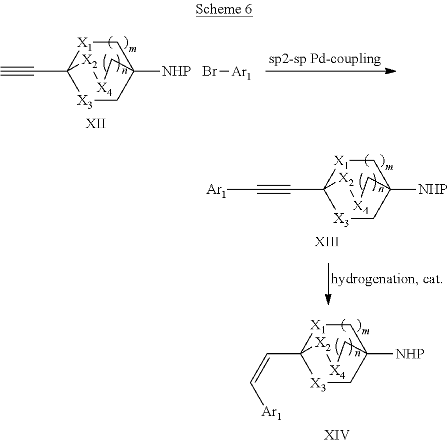

An alternate class of compounds can be prepared starting from the appropriate aryl bromide Br—Ar₁ by performing a halogen-metal exchange using, for example, n-BuLi followed by addition of XV to give XVI (Scheme 7).

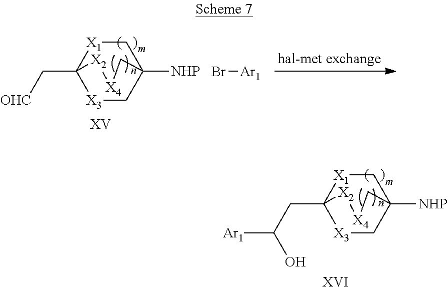

A class of ether linked compounds can be prepared by reacting XVII with HO—Ar₁ and an appropriate base to give XVIII. The ester of XVIII can be converted to the corresponding amine using conditions familiar to those skilled in the art (saponification, followed by Curtius rearrangement) to give XIX (Scheme 8).

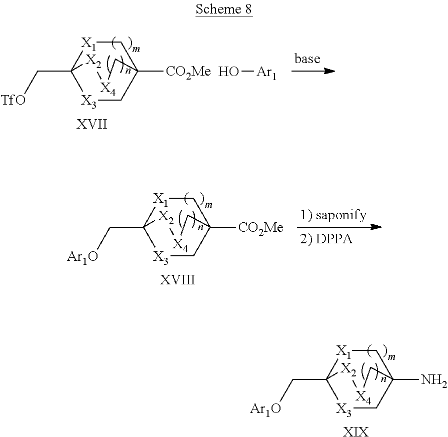

An additional class of compounds can be prepared by performing a reductive amination on XX using ammonia followed by protection of the resultant amine with, for example, CbzCl to give XXI (Scheme 9). Selective deprotection of P₁ followed by transformation as described above and then deprotection of P₂ gives the final products. Alternatively, XX can be converted directly into final products. An additional approach involves reacting the ketone of XX with hydroxylamine or an alkylhydroxylamine to give XXIII, which can be converted to final products using the methods described above.

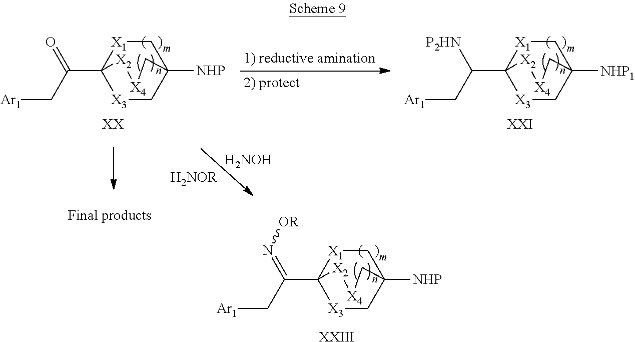

A class of dihydroxy-containing compounds can be prepared from XXIV using, for example, osmium tetroxide, to give XXV, which can be further transformed as described above (Scheme 10).

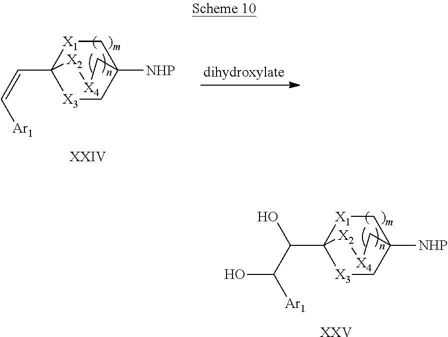

Compounds where Ar₁ contains an acidic —NH within the ring can be prepared by treatment of H₂N—Ar₁ with an appropriate base followed by addition of XXVI to give XXVII (Scheme 11)

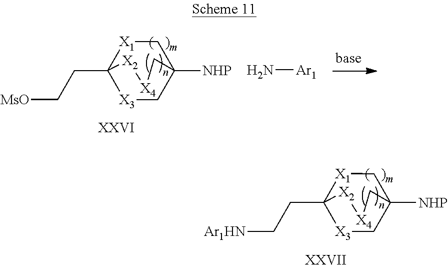

In a closely related transformation, triflate XXVIII can be used to alkylate HN—Ar₁ (Scheme 12).

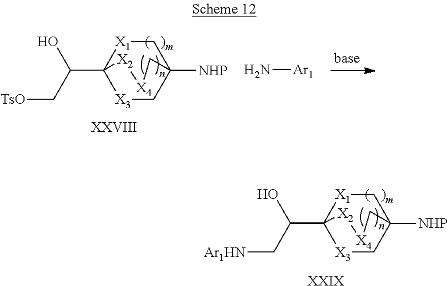

The antibacterial activity of the present compounds can be demonstrated by various assays known in the art, for example, by their minimum inhibitory concentration (MIC-100) against bacteria and minimum effective concentration (MEC). Compounds provided in the Examples were generally found to inhibit the growth of S. aureus in the range of 0.015 to 64 μg/mL.

The potency of antibacterial agents was measured using the Minimal Inhibitory Concentration (MIC) assay. The assay measures the ability of test agents to inhibit the growth of bacteria on agar-containing medium.

The bacterial test strains used were exemplified by Staphylococcus aureus Smith, Enterococcus faecium A2373, Streptococcus pneumoniae IID554, and Escherichia coli ATCC 25922. All strains were maintained as frozen stocks held at −80° C. in skim milk. Other bacterial test strains are well known to those skilled in the art and can be used for testing.

Mueller Hinton Agar (MHA BBL; Becton Dickinson and Company, Sparks, Md.) was used as the medium. MHA was supplemented with 5% defibrinated horse blood (DHB; Nippon Biotest Laboratories inc.) to support the growth of S. pneumoniae and E. faecium.

MIC values were determined using a modified agar dilution procedure described by the Clinical and Laboratory Standards Institute (CLSI; Methods for Dilution Antimicrobial Susceptibility Tests for Bacteria That Grow Aerobically; Approved Standard—Eighth Edition. CLSI document M07-A8 [ISBN 1-56238-689-1]. Clinical and Laboratory Standards Institute, 940 West Valley Road, Suite 1400, Wayne, Pa. 19087-1898 USA, 2009).

Stock solutions (6.4 mgmL) of test compounds were prepared in 100% ultrapure dimethyl sulfoxide (DMSO; source) on the day of the assay. Subsequent serial dilutions were performed to generate solutions with concentrations ranging from 6.4 to 0.0002 mgmL in 100% DMSO.

Agar medium containing test compound was prepared by adding the dilutions of antimicrobial solution to molten MHA at a temperature of 45-50° C. The agar and antimicrobial solution were mixed thoroughly, poured into petri dishes, and allowed to solidify at room temperature. The final concentration of test compounds in the MHA medium ranged from 128 to 0.001 μg/mL with two-fold dilutions. MHA plates lacking antibacterial compound were used for growth controls.

Prior to susceptibility testing, the bacterial isolates were removed from frozen storage, thawed at room temperature, sub-cultured to MHA medium and incubated overnight at 35° C. S. pneumoniae and E. faecium were subcultured on MHA supplemented with 5% DHB at 35° C. with 5%. Colonies from each plate were suspended in normal saline. This suspension was adjusted to the turbidity of a 0.5 McFarland standard, 1-2×10⁸ colony forming units (CFU) per mL, and diluted 100-fold to 1-2×10⁶ CFUmL.

Suspensions of bacterial cultures were applied to the surface of MHA plates containing test compound as well as to a growth control plate lacking test compound using an inoculum-replicating device with 4 mm pins. The replicating device applied 5 uL of the bacterial suspension such that each spot contained approx. 1×10⁴ CFU. Plates were dried for about 40 min and incubated at 35° C. for 16-20 hr prior to scoring. The MIC was recorded as the lowest concentration of test agent that completely inhibited growth.

S. aureus Smith and S. pneumoniae IID554 strains were susceptible to levofloxacin, vancomycin, and linezolid based on MIC interpretive standards defined by CLSI. E. faecium A2373 was susceptible to linezolid but resistant to vancomycin. E. coli ATCC 25922 and Pseudomonas aeruginosa PAO1 were susceptible to levofloxacin and imipenem. All test agents demonstrated potent activity against S. aureus with MIC values ranging from 0.016 to 32 μg/mL. See Table 1. MIC results were slightly higher against E. coli ATCC 25922 (values ranged from 1 to >64 μg/mL, data not shown). Representative compounds, tested against multiple bacteria, demonstrated broad spectrum antibacterial activity. See Table 2.

Example Numbers correspond to the examples described in the Examples section.

TABLE 1

| Example Number | S_aureus_Smith_WT_MIC (μg/mL) |
|---|---|
| 1 | 0.0310 |
| 2 | 0.125 |
| 3 | 0.250 |
| 4 | 0.0160 |
| 5 | 0.0310 |
| 6 | 0.0310 |
| 7 | 0.500 |
| 8 | 0.500 |
| 9 | 0.125 |
| 10 | 0.0160 |
| 11 | 2.00 |
| 12 | 0.250 |
| 13a | 0.0310 |
| 13b | 0.0630 |
| 14a | 0.0310 |
| 14b | 0.0310 |
| 15 | 0.0310 |
| 16 | 0.0160 |
| 17 | 0.0310 |
| 18 | 0.0160 |
| 19 | 0.0080 |
| 20a | 0.0630 |
| 20b | 0.0630 |
| 21a | 0.250 |
| 21b | 0.125 |
| 22 | 0.250 |
| 23 | 0.125 |
| 24 | 4.00 |
| 26a | 0.250 |
| 26b | 1.00 |
| 27a | 0.250 |
| 27b | 2.00 |
| 28 | 0.500 |
| 29 | 0.250 |
| 30 | 2.00 |
| 31 | 0.0630 |
| 32 | 0.250 |
| 33 | 0.0630 |
| 34 | 0.0310 |
| 35 | 0.0160 |
| 36 | 0.0310 |
| 37 | 0.0630 |
| 38 | 0.250 |
| 39 | 0.500 |
| 40 | 0.250 |
| 41 | 0.500 |
| 42 | 0.0630 |
| 43 | 1.00 |
| 44 | 0.0160 |
| 45 | 0.0310 |
| 46 | 2.00 |
| 47 | 2.00 |
| 48 | 2.00 |
| 49 | 1.00 |
| 50 | 0.125 |
| 51a | 0.0310 |
| 51b | 0.0630 |
| 52a | 0.0310 |
| 52b | 0.0630 |
| 53a | 0.500 |
| 53b | 0.250 |
| 54a | 0.250 |
| 54b | 0.0160 |
| 55a | 1.00 |
| 55b | 0.250 |
| 56 | 0.0310 |
| 57 | 4.00 |
| 58 | 0.125 |
| 59 | 0.0160 |
| 60 | 8.00 |
| 61 | 0.063 |
| 62 | 4.00 |
| 63 | 0.250 |
| 64 | 4.00 |
| 65 | 2.00 |
| 66 | 0.125 |
| 67 | 0.0310 |
| 68 | 0.0160 |
| 69 | 0.0080 |
| 70 | 2.00 |
| 71 | 1.00 |
| 72 | 0.125 |
| 73 | 0.0160 |
| 74 | 0.250 |
| 75 | 4.00 |
| 76 | 0.125 |
| 77 | 0.125 |
| 78 | 0.250 |
| 79 | 0.500 |
| 80 | 0.0630 |
| 81 | 0.500 |
| 82 | 0.250 |
| 83 | 1.00 |
| 84 | 0.250 |
| 85 | 0.125 |
| 86 | 16.0 |
| 87 | 0.0630 |
| 88 | 0.0080 |
| 89 | 0.125 |
| 90 | 0.125 |
| 91 | 16.0 |
| 92 | 0.125 |
| 93 | 16.0 |
| 94 | >16.0 |
| 95 | 0.500 |
| 96 | 0.0310 |
| 97 | 1.00 |
| 98 | 0.0160 |
| 99 | 0.250 |
| 100a | 0.250 |
| 100b | 0.0630 |
| 101 | 2.00 |
| 102 | 2.00 |
| 103 | 4.00 |
| 104 | >8.0 |
| 105 | 2.00 |
| 106 | 1.00 |
| 107 | 32.0 |
| 108 | >8.0 |
| 109 | 8.00 |
| 110 | 4.00 |
| 111 | 0.125 |
| 112 | 1.00 |
| 113 | 16.0 |
| 114 | 4.00 |
| 115 | 0.500 |
| 116 | 16.0 |
| 117 | 64.0 |
| 118 | 32.0 |
| 119 | 0.125 |
| 120 | 0.0160 |
| 121 | 0.0160 |
| 122 | 0.0630 |
| 123 | 1.00 |
| 124 | 0.0310 |
| 125 | 0.0160 |
| 126 | 0.250 |
| 127 | 0.250 |
| 128 | 0.0630 |
| 129 | 0.0630 |
| 130 | 0.0630 |
| 131 | 0.0160 |
| 132 | 0.0310 |
| 133 | 1.00 |
| 134 | 0.0630 |
| 135 | 0.0310 |
| 136 | 0.250 |
| 137 | 0.0630 |
| 138 | 0.500 |
| 139 | 0.0630 |
| 140 | 0.0630 |
| 141 | 0.0630 |
| 142 | 0.0310 |
| 143 | 0.0310 |
| 144 | 0.125 |
| 145 | 0.250 |
| 146 | 0.125 |
| 147 | 0.125 |
| 148 | 0.0310 |
| 149 | 0.0630 |
| 150 | 0.500 |
| 151a | 32.0 |
| 151b | 4.00 |
| 152 | 0.250 |
| 153 | 0.0630 |
| 154 | 1.00 |
| 155 | 0.0160 |
| 156 | 0.0630 |
| 157a | 2.00 |
| 157b | 2.00 |
| 158 | 0.125 |
| 159 | 2.00 |
| 160 | 0.250 |
| 161 | 2.00 |
| 162 | 2.00 |
| 163 | 4.00 |
| 164 | 0.500 |
| 165 | 2.00 |
| 166 | 0.500 |
| 167 | 2.00 |
| 168 | 16.0 |
| 169 | 0.0630 |
| 170 | 0.125 |
| 171 | 0.125 |
| 172 | 0.0310 |
| 173 | 0.125 |
| 174 | 0.0310 |
| 175 | 1.00 |
| 176 | 0.125 |
| 177 | 1.00 |
| 178 | 0.0630 |
| 179 | 0.500 |
| 180 | 0.0310 |
| 181 | 0.125 |
| 182 | 4.00 |
| 183 | 1.00 |
| 184 | 0.250 |
| 185 | 0.125 |
| 186 | 0.0630 |
| 188 | 0.016 |
| 189 | 0.016 |
| 190 | 0.063 |
| 191 | 0.016 |
| 192 | 0.031 |
| 193 | 0.031 |
| 194 | 1 |
| 195 | 1 |
| 196 | 0.25 |
| 197 | 0.5 |
| 198 | 2 |
| 199 | 0.25 |
| 200 | 0.75 |
| 201 | 0.06 |
| 202 | 0.25 |
| 203 | 0.25 |
| 204 | 0.06 |
| 205 | 2 |
| 206 | 0.06 |
| 207 | 0.5 |
| 208 | 0.06 |
| 209 | 8 |
| 210 | 0.25 |
| 227 | 0.063 |
| 228 | 0.25 |
| 283 | 16 |
| 288 | 0.125 |
| 292 | 0.125 |
| 293 | 0.5 |
| 294 | 0.125 |

TABLE 2

| | Strep_Pn_{—} | E_coli_{—} | P_ae_{—} | A_bau_{—} |
|---|---|---|---|---|
| | IID554_WT_{—} | ATCC25922_{—} | PAO1_{—} | IID876_{—} |
| | MIC_{—} | WT_MIC_{—} | WT_MIC_{—} | WT_MIC_{—} |
| Example | μg/mL | μg/mL | μg/mL | μg/mL |
| 18 | 0.0630 | 1.00 | 4.00 | 0.500 |
| 20a | 0.250 | 4.00 | 16.0 | 1.00 |

The following examples serve only to illustrate the invention and its practice. The examples are not to be construed as limitations on the scope or spirit of the invention.

### ABBREVIATIONS

9-BBN 9-Borabicyclo(3.3.1)nonane
AcOH Acetic acid
Boc t-Butyloxycarbonyl
Boc₂O di-t-Butyl dicarbonate
BuLi n-Butyllithium

### ButOH Butanol

Cat. Catalyst
Cbz Benzyloxycarbonyl (also CBz)

### CH₃CN Acetonitrile

### CH₂Cl₂ Dichloromethane

CsOAc Cesium carbonate

### DMA Dimethylacetamide

### DME Dimethoxyethane

### DCE Dichloroethane

### DCM Dichloromethane

### DMF N,N-Dimethylformamide

DMS Dimethyl sulfide
DMSO Dimethyl sulfoxide
DPPA Diphenyl phosphoryl azide

### Et Ethyl

EtOAc or EA Ethyl acetate

### EtOH Ethanol

Et₂O Diethyl ether

### Et₃N Triethylamine

EMME Diethyl ethoxymethylenemalonate
H₂ Hydrogen or hydrogen atmosphere

### H₂O Water

HOAc Acetic acid
H₂O₂ Hydrogen peroxide
H₂SO₄ Sulfuric acid

### HCHO Formaldehyde

HCl Hydrochloric acid

### HMPA Hexamethylphosphoramide

IBX 2-(Iodoxybenzoic acid
K₂CO₃ Potassium carbonate
KHMDS Potassium hexamethyldisilazide
LAHLiAlH₄ Lithium aluminum hydride (LiAlH₄)
LiCl Lithium chloride
LiHMDS Lithium hexamethyldisilazide
LDA Lithium diisopropyl amide
MCPBA meta-Chloroperoxybenzoic acid (m-CPBA)

### Me Methyl

### MeOH Methanol

MsCl Methanesulfonyl chloride
NaBH₄ Sodium borohydride
NaCl Sodium chloride
NaH Sodium hydride
NaIO₄ Sodium periodate
NaOH Sodium hydroxide

### NCS N-chlorosuccinimide

NH₄Cl Ammonium chloride
Na₂SO₄ Sodium sulfate
NMM N-Methyl morpholine

### NMO 4-Methylmorpholine N-oxide

NMP N-Methyl pyrrolidinone
NOBF₄ Nitrosyl tetrafluoroborate

### O₃ Ozone

OSO₄ Osmium tetroxide

### Pd Palladium

PDC Pyridinium dichromate

### PE Petroleum Ether

### Ph Phenyl

RT or r.t. Room temperature, approximately 25° C.
SeO₂ Selenium dioxide
SOCl₂ Thionyl chloride
t-BuOH tert-Butanol
t-BuOK Potassium t-butoxide
TBAB Tetrabutylammonium bromide
TBME tert-Butyl methyl ether
TsCl Toluenesulfonyl chloride
TsOH Toluenesulfonic acid hydrate

### TEA Triethanolamine

Tf₂O Triflic anhydride
TFA Trifluoroacetic acid

### THF Tetrahydrofuran

TLC Thin layer chromatography
TMSCl Trimethysilyl chloride

### Preparation of Intermediates

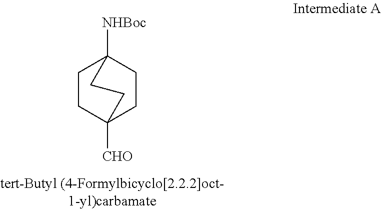 [SMILES: CC(C)(C)OC(=O)NC12CCC(C=O)(CC1)CC2]

Step 1 and 2

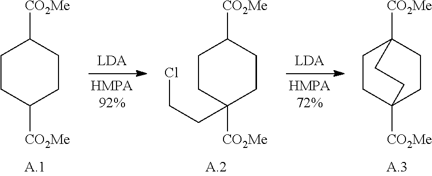 [SMILES: COC(=O)C1CCC(C(=O)OC)CC1 ; COC(=O)C1CCC(CCCl)(C(=O)OC)CC1 ; COC(=O)C12CCC(C(=O)OC)(CC1)CC2]

To a solution of diisopropylamine (42.0 mL) in anhydrous tetrahydrofuran (350 mL) was added a solution of butyllithium (174.0 mL, 1.58 M in hexane) at −15° C., the mixture was stirred at −10° C. for 15 minutes. Hexamethylphosphoramide (174.0 mL) was added to the mixture at −60° C. To a resulting mixture was added a solution of dimethyl cyclohexanedicarboxylate (50.00 g) in anhydrous tetrahydrofuran (50 mL) at −65° C., the mixture was stirred at the same temperature for 1 hour. 1-Bromo-2-chloroethane (25.0 mL) was added to the mixture at −65° C., the resulting mixture was stirred at the same temperature for 1 hour, and further stirred at the room temperature for 1 hour. After quenching the reaction by adding saturated ammonium chloride solution (125 mL), the mixture was concentrated in vacuo. After diluting the residue with water, the mixture was extracted with hexane. The organic extracts were washed with water, dried over anhydrous sodium sulfate, filtered, and then concentrated in vacuo to give A.2 (60.20 g).

To a solution of diisopropylamine (33.8 mL) in anhydrous tetrahydrofuran (310 mL) was added butyllithium (140.0 mL, 1.58 M in hexane) at −15° C., the mixture was stirred at −10° C. for 15 minutes. To a solution of A.2 (crude, 55.17 g) and hexamethylphosphoramide (146.0 mL) was added a lithium diisopropyl amide solution prepared as above at −65° C., the resulting mixture was stirred at the same temperature for 1 hour, and further stirred at the room temperature for 3 hours. After quenching the reaction by adding saturated ammonium chloride solution (170 mL), the mixture was concentrated in vacuo. After diluting the residue with water (800 mL), the resulting precipitates were collected by filtration, washed with water and dried in vacuo to give the crude product (40.5 g).

Another experiment at the same reaction scale gave the crude product (42.6 g).

Flash chromatography (hexane:ethyl acetate=4:1) of the combined crude product (83.1 g) gave A.3 (68.86 g).

¹H NMR (CDCl₃): δ 1.81 (s, 12H), 3.65 (s, 6H).

Step 3

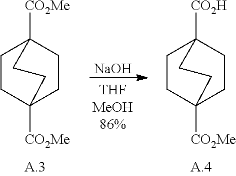

To a solution of A.3 (149.2 g) in anhydrous tetrahydrofuran (2.2 L) was added a solution of sodium hydroxide (264 mL, 2.5 M in methanol) at room temperature, the mixture was stirred at the same temperature for 15.5 hours. The insoluble materials (material A) were collected by filtration and washed with tetrahydrofuran. The combined filtrate and washing were concentrated in vacuo. After dilution of the residue with water, the mixture was washed with hexane. To the aqueous solution was added material A obtained above, the mixture was washed with hexane and adjusted to pH 1 by addition of concentrated hydrochloric acid under cooling with ice. The mixture was extracted with ethyl acetate. The organic extracts were washed with brine, dried over anhydrous sodium sulfate, filtered, and then concentrated in vacuo to give A.4 (120.4 g).

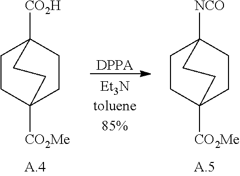

Step 4

To a suspension of A.4 (4.00 g) in anhydrous toluene (94 mL) was added triethylamine (2.89 mL) and diphenyl phosphoryl azide (4.47 mL), the mixture was stirred at room temperature for 2 hours and heated at reflux for 2 hours. The reaction mixture was washed with 10% citric acid solution, saturated sodium hydrogencarbonate solution, water and brine. The organic extracts were dried over anhydrous sodium sulfate, filtered, and then concentrated in vacuo. Flash chromatography (hexane:ethyl acetate=8:1) of the residue gave A.5 (3.35 g).

¹H NMR (CDCl₃): δ 1.80-1.85 (m, 6H), 1.90-1.92 (m, 6H), 3.64 (s, 3H).

Step 5

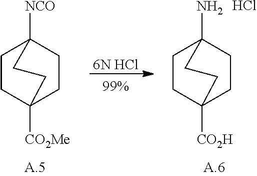

A suspension of A.5 (2.73 g) in 6 N hydrochloric acid (39.3 mL) was heated under reflux for 5 hours, the mixture was concentrated in vacuo to give A.6 (2.67 g).

¹H NMR (DMSO-d₆): δ 1.68-1.80 (m, 12H), 11.6 (br, 3H).

Step 6

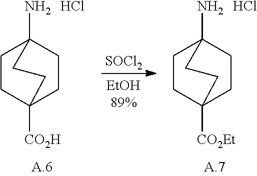

Thionyl chloride (0.15 mL) was added to anhydrous ethanol (3 mL) under cooling with ice, the resulting mixture was added A.6 (206 mg) at room temperature. The mixture was heated under reflux for 3 hours and concentrated in vacuo to give A.7 (208 mg).

¹H NMR (DMSO-d₆): δ 1.14 (t, J=7.3 Hz, 3H), 1.71-1.80 (m, 12H), 4.01 (q, J=7.3 Hz, 2H), 8.21 (br, 3H).

Step 7

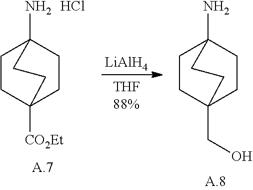 [SMILES: Cl ; CCOC(=O)C12CCC(N)(CC1)CC2 ; "LiAlH4" ; NC12CCC(CO)(CC1)CC2]

To a solution of lithium aluminum hydride (400 mL, 1.0 M solution in diethyl ether) in anhydrous tetrahydrofuran (400 mL) was added A.7 (46.74 g) at −20° C., the mixture was stirred at room temperature for 5 hours. After quenching the reaction by adding water-tetrahydrofuran (1:1, 72 mL) at −20° C., and 5 N sodium hydroxide solution (18 mL) at −5° C., the mixture was stirred at room temperature for 30 minutes. The insoluble materials were filtered off and washed with dichloromethane/methanol (5:1, 300 mL). The combined filtrate and the washing were concentrated in vacuo to give A.8 (33.68 g).

¹H NMR (CDCl₃): δ 1.43-1.54 (m, 12H), 3.27 (s, 2H).

Step 8

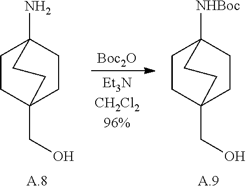 [SMILES: "Boc2O" ; NC12CCC(CO)(CC1)CC2 ; CC(C)(C)OC(=O)NC12CCC(CO)(CC1)CC2]

To a solution of A.8 (15.00 g) in dichloromethane (140 mL) was added a solution of di-tert-butyl dicarbonate (18.78 g) in dichloromethane (16 mL) and triethylamine (12.0 mL) at 4° C., the mixture was stirred at the same temperature for overnight. The mixture was washed with 10% citric acid solution, saturated sodium hydrogencarbonate solution and brine, dried over anhydrous sodium sulfate, filtered, and then concentrated in vacuo. Treatment of the residue with diisopropyl ether gave A.9 (19.09 g).

¹H NMR (CDCl₃): δ 1.22 (t, J=5.5 Hz, 1H), 1.42 (s, 9H), 1.45-1.55 (m, 6H), 1.77-1.88 (m, 6H), 3.26 (d, J=5.5 Hz, 2H), 4.33 (s, 1H).

Step 9

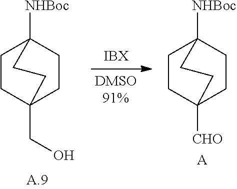

To a solution of A.9 (2.00 g) in dimethyl sulfoxide (31 mL) was added 2-iodoxybenzoic acid (3.29 g) at room temperature, the resulting suspension was stirred at the same temperature for 1 hour. After dilution of the mixture with water, the mixture was extracted with ethyl acetate. The organic extracts were dried over anhydrous sodium sulfate, filtered, and then concentrated in vacuo. Flash chromatography (hexane:ethyl acetate=6:1) of the residue gave A (1.81 g).

¹H NMR (CDCl₃): δ 1.42 (s, 9H), 1.69-1.77 (m, 6H), 1.81-1.96 (m, 6H), 4.37 (s, 1H), 9.44 (s, 1H).

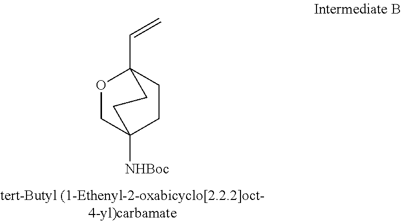 [SMILES: C=CC12CCC(NC(=O)OC(C)(C)C)(CC1)CO2]

Step 1

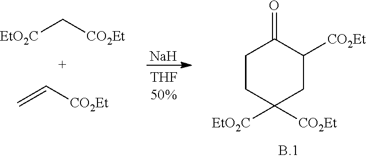

A suspension of sodium hydride (112.3 g) in anhydrous tetrahydrofuran (1 L) was added a solution of diethyl malonate (150 g) in anhydrous tetrahydrofuran (300 mL) at 40-45° C., the suspension was stirred at the same temperature for 15 minutes. A solution of ethyl acrylate (215 mL) in anhydrous tetrahydrofuran (300 mL) was added to the suspension, the resulting mixture was stirred for 15 minutes. The mixture was poured onto ice water, adjusted to pH 3 by addition of concentrated hydrochloric acid and extracted with ethyl acetate. The organic extracts were dried over anhydrous sodium sulfate, filtered, and then concentrated in vacuo. Flash chromatography (hexane:ethyl acetate=9:1→6:1→4:1) of the residue gave B.1 (147.8 g).

¹H NMR (CDCl₃): δ 1.23-1.33 (m, 9H), 2.34-2.46 (m, 6H), 4.19-4.28 (m, 6H).

Step 2

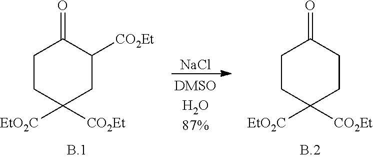 [SMILES: CCOC(=O)C1CC(C(=O)OCC)(C(=O)OCC)CCC1=O ; "NaCl" ; CCOC(=O)C1(C(=O)OCC)CCC(=O)CC1]

A mixture of B.1 (158.4 g) and sodium chloride (86.3 g) in dimethyl sulfoxide (720 mL) and water (21.6 mL) was heated at 160° C. for 1.7 hours. The mixture was poured onto ice water and extracted with ethyl acetate. The organic extracts were dried over anhydrous sodium sulfate, filtered, and then concentrated in vacuo. Flash chromatography (hexane:ethyl acetate=3:1) of the residue gave B.2 (111.7 g).

¹H NMR (CDCl₃): δ 1.24-1.30 (m, 6H), 2.34-2.48 (m, 8H), 4.25 (q, J=7.4 Hz, 4H).

Step 3

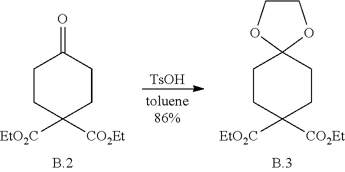 [SMILES: CCOC(=O)C1(C(=O)OCC)CCC(=O)CC1 ; "TsOH" ; CCOC(=O)C1(C(=O)OCC)CCC2(CC1)OCCO2]

A mixture of B.2 (105.5 g), ethylene glycol (29.1 mL) and toluenesulfonic acid hydrate (827 mg) in toluene (870 mL) was heated under reflux for 4 hours with using Dean-Stark apparatus. The mixture was poured onto saturated sodium hydrogencarbonate solution and extracted with ethyl acetate. The organic extracts were dried over anhydrous sodium sulfate, filtered, and then concentrated in vacuo. Flash chromatography (hexane:ethyl acetate=5:1) of the residue gave B.3 (106.6 g).

¹H NMR (CDCl₃): δ 1.25 (t, J=7.3 Hz, 6H), 1.69 (t, J=6.1 Hz, 4H), 2.18 (t, J=6.1 Hz, 4H), 3.94 (s, 4H), 4.18 (q, J=7.3 Hz, 4H).

Step 4

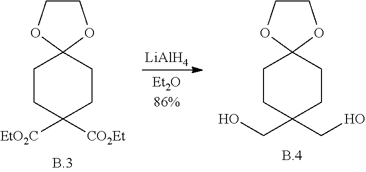 [SMILES: CCOC(=O)C1(C(=O)OCC)CCC2(CC1)OCCO2 ; "LiAlH4" ; OCC1(CO)CCC2(CC1)OCCO2]

To a solution of lithium aluminum hydride (738 mL, 1 M in diethyl ether) was added a solution of B.3 (105.7 g) in anhydrous diethyl ether (738 mL) at −20° C., the resulting suspension was stirred at 0° C. for 3 hours. After quenching the reaction by adding water-tetrahydrofuran (1:1, 132.8 mL) and 5 N sodium hydroxide solution (33.2 mL) under cooling with ice, the mixture was stirred at room temperature for overnight. After dilution of the mixture with dichloromethane-methanol (5:1, 1 L), the insoluble materials were filtered off and washed with dichloromethane-methanol (5:1, 500 mL×2). The combined mixture of the filtrate and washing was added silica-gel (220 g). The suspension was stirred for 15 minutes. The insoluble materials were filtered off and washed with (dichloromethane:methanol=5:1). The combined filtrate and the washing were concentrated in vacuo to give B.4 (64.0 g).

¹H NMR (CDCl₃): δ 1.53-1.58 (m, 4H), 1.60-1.65 (m, 4H), 2.37 (t, J=5.5 Hz, 2H), 3.65 (d, J=5.5 Hz, 4H), 3.95 (s, 4H).

Step 5

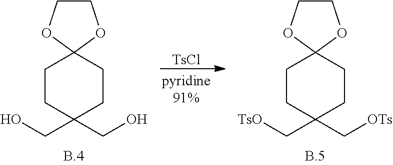

To a solution of B.4 (112.0 g) in anhydrous pyridine (700 mL) was added toluenesulfonyl chloride (232.3 g) under cooling with ice, the resulting suspension was stirred at room temperature for overnight. After dilution of the mixture with ethyl acetate, the mixture was washed with 10% aqueous citric acid solution (1 L×4) and brine. The organic extracts were concentrated in vacuo. Treatment of the residue with ethanol (1.5 L) gave B.5 (343.5 g).

¹H NMR (CDCl₃): δ 1.46-1.52 (m, 8H), 2.46 (s, 6H), 3.84 (s, 4H), 3.88 (s, 4H), 7.35 (d, J=8.0 Hz, 4H), 7.71-7.76 (m, 4H).

Step 6

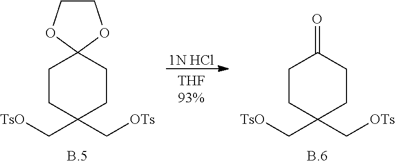 [SMILES: Cc1ccc(S(=O)(=O)OCC2(COS(=O)(=O)c3ccc(C)cc3)CCC3(CC2)OCCO3)cc1 ; "1N HCl" ; Cc1ccc(S(=O)(=O)OCC2(COS(=O)(=O)c3ccc(C)cc3)CCC(=O)CC2)cc1]

A mixture of B.5 (240.1 g), 1 N hydrochloric acid (1.8 L) and tetrahydrofuran (3.6 L) was heated under reflux for 5 hours. The mixture was extracted with ethyl acetate. The organic extracts were washed with water, dried over anhydrous sodium sulfate, filtered, and then concentrated in vacuo to give the crude product. A suspension of the crude product in hexane (1 L) was stirred at room temperature for 30 minutes. The precipitates were collected by filtration to give B.6 (219.0 g).

¹H NMR (CDCl₃): δ 1.72 (t, J=7.3 Hz, 4H), 2.22 (t, J=7.3 Hz, 4H), 2.47 (s, 6H), 3.94 (s, 4H), 7.37 (d, J=7.9 Hz, 4H), 7.72-7.76 (m, 4H).

Step 7

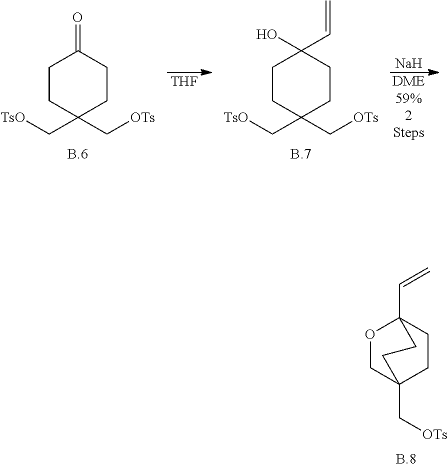

To a solution of vinylmagnesium bromide (203 mL, 1 M in tetrahydrofuran) was added drop wise a solution of B.6 (73.0 g) in anhydrous tetrahydrofuran (312 mL) at −78° C. for 5 hours, the mixture was stirred at the same temperature for 15 minutes. After quenching the reaction by adding saturated ammonium chloride solution, the mixture was evaporated in vacuo to remove tetrahydrofuran. The mixture was extracted with diethyl ether. The organic extracts were washed with brine, dried over anhydrous sodium sulfate, filtered, and then concentrated in vacuo to give the crude alcohol B.7.

¹H NMR (CDCl₃): δ 1.36-1.46 (m, 8H), 2.46 (s, 3H), 2.47 (s, 3H), 3.76 (s, 2H), 3.92 (s, 2H), 5.05 (d, J=11.0 Hz, 1H), 5.18 (d, J=18.4 Hz, 1H), 5.85 (dd, J=17.8, 11.0 Hz, 1H), 7.32-7.38 (m, 4H), 7.70-7.77 (m, 4H).

To a solution of B.7 in anhydrous 1,2-dimethoxyethane (3.2 L) was added sodium hydride (22.5 g, 50% in mineral oil) under cooling with ice, the mixture was stirred at the same temperature for 30 minutes. The mixture was heated under reflux for 2.5 hours. After quenching the reaction by adding saturated ammonium chloride solution, the mixture was extracted with ethyl acetate. The organic extracts were dried over anhydrous sodium sulfate, filtered, and then concentrated in vacuo. Flash chromatography (hexane:ethyl acetate=3:1) of the residue gave B.8 (26.7 g).

¹H NMR (CDCl₃): δ 1.47-1.53 (m, 2H), 1.60-1.72 (m, 4H), 1.82-1.92 (m, 2H), 2.45 (s, 3H), 3.66-3.68 (m, 2H), 3.69 (s, 2H), 5.01 (dd, J=11.0, 1.2 Hz, 1H), 5.12 (dd, J=17.8, 1.2 Hz, 1H), 5.78 (dd, J=17.1, 11.0 Hz, 1H), 7.35 (d, J=8.0 Hz, 2H), 7.76 (d, J=8.0 Hz, 1H).

Step 8

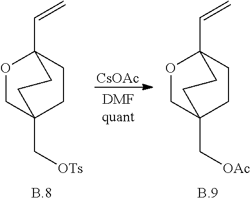 [SMILES: C=CC12CCC(COS(=O)(=O)c3ccc(C)cc3)(CC1)CO2 ; C=CC12CCC(COC(C)=O)(CC1)CO2 ; "CsOAc"]

A mixture of B.8 (27.0 g) and cesium carbonate (52.7 g) in anhydrous N,N-dimethylformamide (500 mL) was heated at 100° C. for overnight. After dilution of the mixture with water, the mixture was extracted with ethyl acetate. The organic extracts were dried over anhydrous sodium sulfate, filtered, and then concentrated in vacuo to give B.9 (17.7 g).

¹H NMR (CDCl₃): δ 1.52-1.62 (m, 2H), 1.66-1.77 (m, 4H), 1.85-1.95 (m, 2H), 2.05 (s, 3H), 3.79-3.81 (m, 4H), 5.03 (dd, J=11.0, 1.8 Hz, 1H), 5.15 (dd, J=17.8, 1.2 Hz, 1H), 5.82 (dd, J=17.7, 1.8 Hz, 1H).

Step 9

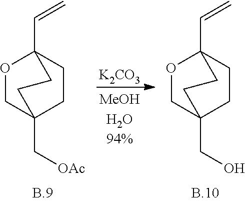

To a solution of B.9 (17.0 g) in methanol (265 mL) was added a solution of potassium carbonate (55.8 g) in water (340 mL) under cooling, the mixture was stirred at room temperature for 2 hours and was evaporated in vacuo to remove methanol. The aqueous mixture was extracted with ethyl acetate. The organic extracts were dried over anhydrous sodium sulfate, filtered, and then concentrated in vacuo. Flash chromatography (hexane:ethyl acetate=1:2) of the residue gave B.10 (13.9 g).

¹H NMR (CDCl₃): δ 1.49-1.59 (m, 2H), 1.64-1.76 (m, 4H), 1.85-1.95 (m, 2H), 3.35 (d, J=5.5 Hz, 2H), 3.81-3.82 (m, 2H), 3.79-3.81 (m, 4H), 5.02 (dd, J=11.0, 1.2 Hz, 1H), 5.16 (dd, J=17.8, 1.2 Hz, 1H), 5.82 (dd, J=17.8, 11.0 Hz, 1H).

Step 10

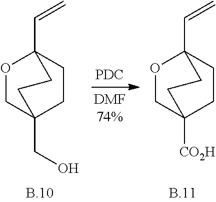

To a solution of B.10 (22.7 g) in N,N-dimethylformamide (360 mL) was added pyridinium dichromate (177.8 g) under cooling with ice, the mixture was stirred at 25-40° C. for 3.5 hours. After dilution of the mixture with water, the mixture was extracted with ethyl acetate. The organic extracts were extracted with 1 N potassium hydroxide solution. The aqueous solution was adjusted to pH 1 by adding concentrated hydrochloric acid and extracted with ethyl acetate. The organic extracts were dried over anhydrous sodium sulfate, filtered, and then concentrated in vacuo. Flash chromatography (hexane:ethyl acetate:acetic acid=1:1:0.02) of the residue gave B.11 (18.1 g).

¹H NMR (DMSO-d₆): δ 1.67-1.87 (m, 8H), 3.83 (s, 2H), 4.96 (dd, J=11.0, 1.8 Hz, 1H), 5.08 (dd, J=17.8, 1.8 Hz, 1H), 5.77 (dd, J=17.7, 11.0 Hz, 1H).

Step 11

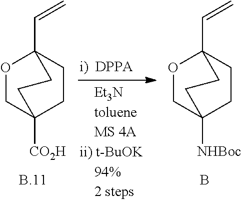

To a suspension of B.11 (10.0 g) and dried molecular sieves (4 Å, 11.0 g, powder) in anhydrous toluene (280 mL) was added triethylamine (8.42 mL) and diphenyl phosphoryl azide (13.0 mL), the mixture was stirred at room temperature for 2 hours and heated at reflux for 2 hours. After insoluble materials were filtered off, the filtrate was concentrated in vacuo. To a solution of the residue in anhydrous tetrahydrofuran (230 mL) was added potassium tert-butoxide (13.6 g) under cooling with ice, the mixture was stirred at room temperature for overnight. After quenching the reaction by addition of 10% aqueous citric acid solution, the mixture was concentrated in vacuo. After dilution of the residue with ethyl acetate, the mixture was washed with saturated sodium hydrogencarbonate solution, water and brine, dried over anhydrous sodium sulfate, filtered, and then concentrated in vacuo. Flash chromatography (toluene:tetrahydrofuran=10:1) of the residue gave B (13.12 g).

¹H NMR (CDCl₃): δ 1.42 (s, 9H), 1.61-2.01 (m, 6H), 2.06-2.12 (m, 2H), 3.99 (s, 2H), 4.28 (s, 1H), 5.02 (dd, J=11.0, 1.2 Hz, 1H), 5.15 (dd, J=17.8, 1.8 Hz, 1H), 5.81 (dd, J=17.8, 11.0 Hz, 1H).

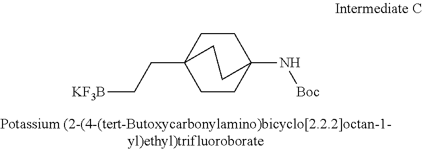

Step 1

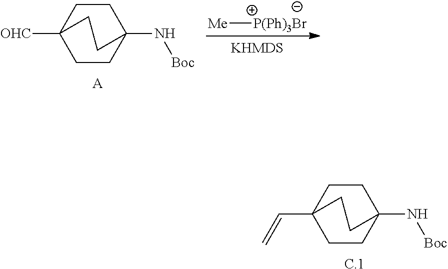

To a suspension of methyltriphenylphosphonium bromide (6.02 g) in toluene (95 mL) was added potassium hexamethyldisilazide (33.7 mL, 0.5 M toluene solution) under cooling with ice, the mixture was stirred at the same temperature for 15 minutes. To the resulting solution was added A (1.78 g), the mixture was stirred at the same temperature for 2 hours. The mixture washed with saturated ammonium chloride solution. The organic extracts were washed with brine, dried over anhydrous sodium sulfate, filtered, and then concentrated in vacuo. Flash chromatography (silica, hexane:ethyl acetate=10:1) of the residue gave C.1 (1.53 g).

¹H NMR (CDCl₃): δ 1.42 (s, 9H), 1.51-1.64 (m, 6H), 1.81-1.90 (m, 6H), 4.32 (br, 1H), 4.82-4.91 (m, 2H), 5.71 (dd, J=18.3, 11.0 Hz, 1H).

MS (CI⁺) m/z: 252 (MH⁺).

HRMS (CI⁺) for C₁₅H₂₆NO₂ (MH⁺): calcd, 252.1964. found, 252.1948.

Step 2

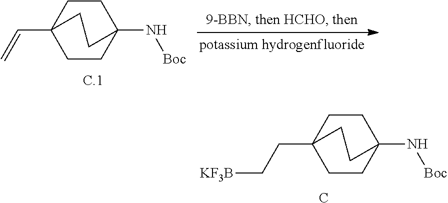

To a solution of C.1 (8.50 g) in tetrahydrofuran (42 mL) was added a solution of 9-borabicyclo(3.3.1)nonane dimer (162 mL, 0.5 M in tetrahydrofuran) under cooling with ice, the mixture was stirred at the same temperature for 20 minutes. After quenching the reaction by adding water (41 mL) under cooling with ice, the mixture was added a solution of formaldehyde (11.1 mL, 37 wt % in water), and the mixture was stirred at room temperature for overnight. After dilution of the mixture with brine, the mixture was extracted with ethyl acetate. The organic extracts were concentrated in vacuo. A solution of the residue in acetone (280 mL) and water (23 mL) was added potassium hydrogen fluoride (26.4 g) under cooling with ice, the mixture was stirred at room temperature for 4 hours, and then concentrated in vacuo. After washing the residue with hexane and diethyl ether, the insoluble materials were extracted with acetone-methanol (5:1) by Soxhlet extractor to give potassium C (4.22 g).

¹H NMR (DMSO-d₆): δ −0.35-0.24 (m, 2H), 0.81-0.91 (m, 2H), 1.23-1.29 (m, 6H), 1.34 (s, 9H), 1.59-1.66 (m, 6H), 6.17 (br, 1H).

MS (FAB⁺) m/z: 360 (MH⁺).

HRMS (FAB⁺) for C₁₅H₂₇BF₃KNO₂ (MH⁺): calcd, 360.1724. found, 360.1711.

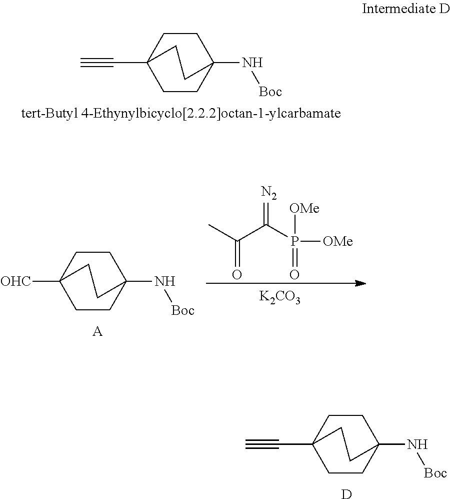

To a solution of dimethyl 1-diazo-2-oxopropylphosphonate (15.2 g) in dichloromethane (400 mL) was added potassium carbonate (8.73 g) and a solution of A (10.0 g) in methanol (400 mL) under cooling with ice, the mixture was stirred at room temperature for 4.5 hours. After quenching the reaction by adding saturated ammonium chloride solution under cooling with ice, the organic extracts were washed with saturated ammonium chloride solution and water, dried over anhydrous sodium sulfate, filtered, and then concentrated in vacuo. Flash chromatography (silica, hexane:ethyl acetate=6:1) of the residue gave D (7.70 g).

¹H NMR (DMSO-d₆): δ 1.44 (s, 9H), 1.77-1.91 (m, 12H), 4.29 (br, 1H).

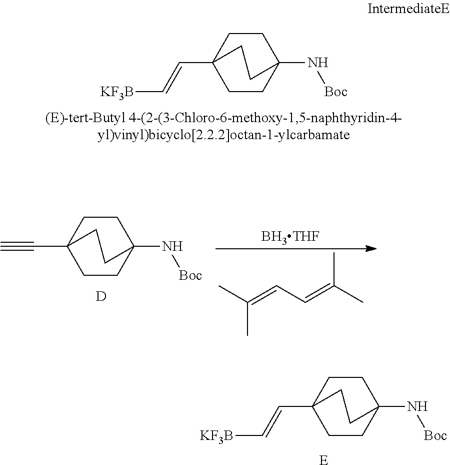

To a solution of 2,5-dimethylhexa-2,4-diene (7.42 g) in tetrahydrofuran (29 mL) was added borane-tetrahydrofuran complex (33.7 mL) under cooling with ice, the mixture was stirred at the same temperature for 3 hours. A solution of D (3.50 g) in tetrahydrofuran (11 mL) was added to the resulting solution of in situ generated Snieckus reagent. The mixture was stirred for 6 hours under cooling with ice. After quenching the reaction by adding water (17.5 mL), formaldehyde (4.2 mL) was added to the mixture. The mixture was stirred at room temperature for 12 hours. After dilution of the mixture with ethyl acetate, the mixture was washed with brine. The organic extracts were dried over anhydrous sodium sulfate, filtered, and then concentrated in vacuo. A solution of the residue in acetone (120 mL) and water (10 mL) was added potassium hydrogenfluoride (11.0 g) under cooling with ice, the mixture was stirred at room temperature for 6 hours, and then concentrated in vacuo. After washing the residue with hexane, the insoluble materials were extracted with acetonemethanol (5:1) by Soxhlet extractor to give Intermediate E (4.63 g).

¹H NMR (DMSO-d₆): δ 1.34 (s, 9H), 1.36-1.42 (m, 6H), 1.62-1.71 (m, 6H), 5.02 (dq, J=18.3, 3.7 Hz, 1H), 5.36 (d, J=18.3 Hz, 1H).

MS (FAB⁺) m/z: 358 (MH⁺).

HRMS (FAB⁺) for C₁₅H₂₅BF₃KNO₂ (MH⁺): calcd, 358.1568. found, 358.1559.

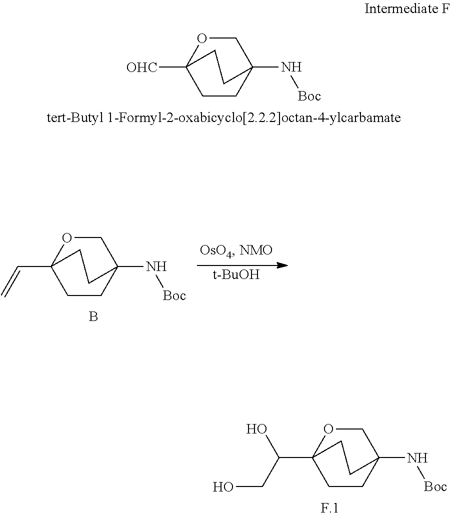

Step 1

To a solution of B (5.00 g) in acetone (84.3 mL) and water (16.9 mL) were added a solution of 4-methylmorpholine N-oxide (20.6 mL, 4.8 M in water) and a solution of osmium tetroxide (10.0 mL, 2.5 wt % in tert-butanol), the mixture was stirred at room temperature for 5 hours. After quenching the reaction by adding a solution of sodium sulfite (73 mL, 17 wt % in water), the mixture was concentrated in vacuo. After dilution of the residue with ethyl acetate, the mixture was washed with water and brine, dried over anhydrous sodium sulfate, filtered, and then concentrated in vacuo. Treatment of the residue with ether gave F.1 (5.18 g).

¹H NMR (CDCl₃): δ 1.42 (s, 9H), 1.60-1.69 (m, 2H), 1.75-1.85 (m, 2H), 1.96-2.17 (m, 4H), 2.38 (dd, J=8.6, 3.7 Hz, 1H), 2.55 (d, J=6.1 Hz, 1H), 3.39-3.45 (m, 1H), 3.60-3.72 (m, 2H), 3.93 (dd, J=7.9, 3.1 Hz, 1H), 3.98 (dd, J=7.9, 2.4 Hz, 1H), 4.28 (br, 1H).

MS (CI⁺) m/z: 288 (MH⁺).

HRMS (CI⁺) for C₁₄H₂₆NO₅ (MH⁺): calcd, 288.1811. found, 288.1818.

Step 2

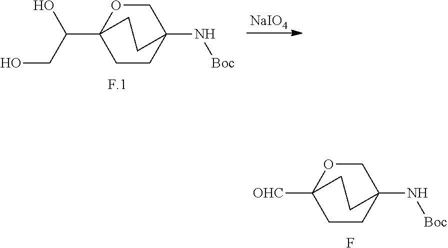

To a solution of F.1 (3.00 g) in tetrahydrofuran (131 mL) was added sodium periodate, the resulting mixture was stirred at room temperature for 30 minutes. After dilution of the mixture with water, the mixture was extracted with ethyl acetate. The organic extracts were washed with brine, dried over anhydrous sodium sulfate, filtered, and then concentrated in vacuo. Treatment of the residue with ether gave F (2.33 g).

¹H NMR (CDCl₃): δ 1.45 (s, 9H), 1.81-1.91 (m, 4H), 1.94-2.06 (m, 2H), 2.07-2.17 (m, 2H), 4.06 (s, 2H), 4.31 (br, 1H), 9.56 (s, 1H).

MS (CI⁺) m/z: 256 (MH⁺).

HRMS (CI⁺) for C₁₃H₂₂NO₄ (MH⁺): calcd, 256.1549. found, 256.1537.

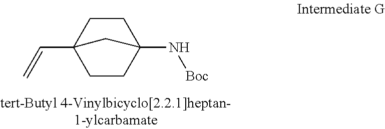 [SMILES: C=CC12CCC(NC(=O)OC(C)(C)C)(CC1)C2]

Step 1

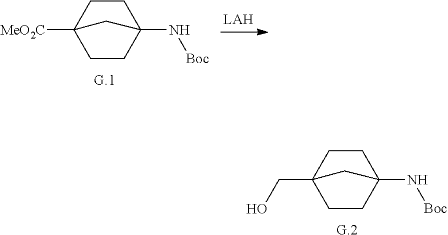

To a solution of methyl G.1 (1.00 g) in tetrahydrofuran (7.4 mL) was added a solution of lithium aluminum hydride (3.71 mL, 1 M in diethyl ether) at −78° C., the mixture was stirred at the same temperature for 6 hours. After quenching the reaction with water and 5 M sodium hydroxide solution, the insoluble materials were filtered off. The filtrate was concentrated in vacuo. Flash chromatography (silica, hexane:ethyl acetate=1:2) of the residue gave G.2 (803 mg).

¹H NMR (CDCl₃): δ 1.25 (t, J=5.5 Hz, 1H), 1.37-1.48 (m, 2H), 1.44 (s, 9H), 1.62-1.91 (m, 8H), 3.64 (d, J=6.1 Hz, 2H), 4.75 (br, 1H).

MS (CI⁺) m/z: 242 (MH⁺).

HRMS (CI⁺) for C₁₃H₂₄NO₃ (MH⁺): calcd, 242.1756. found, 242.1767.

Step 2

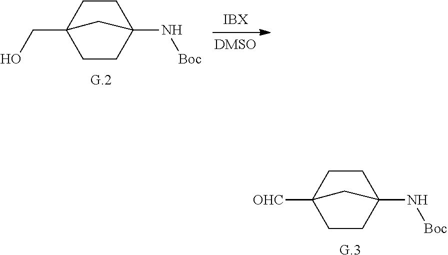

The title compound G.3 (675 mg) was prepared from G.2 (750 mg) in the same manner as described for the synthesis of A.

¹H NMR (CDCl₃): δ 1.45 (s, 9H), 1.49-1.60 (m, 2H), 1.70-1.74 (m, 2H), 1.91 (s, 2H), 2.00-2.12 (m, 4H), 4.76 (br, 1H), 9.75 (s, 1H).

MS (CI⁺) m/z: 240 (MH⁺).

HRMS (CI⁺) for C₁₃H₂₂NO₃ (MH⁺): calcd, 240.1600. found, 240.1599.

Step 3

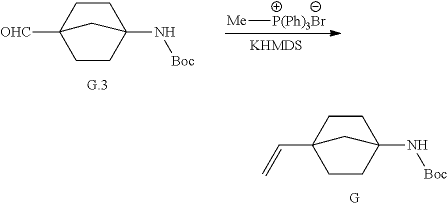

Compound G (440 mg) was prepared from G.3 (649 mg) in the same manner as described for the synthesis of C.1.

¹H NMR (CDCl₃): δ 1.44 (s, 9H), 1.44-1.50 (m, 2H), 1.70-1.90 (m, 8H), 4.74 (br, 1H), 4.95 (dd, J=11.0, 1.8 Hz, 1H), 4.99 (dd, J=17.1, 1.8 Hz, 1H), 5.98 (dd, J=17.2, 11.0 Hz, 1H).

MS (CI⁺) m/z: 238 (MH⁺).

HRMS (CI⁺) for C₁₄H₂₄NO₂ (MH⁺): calcd, 238.1807. found, 238.1837.

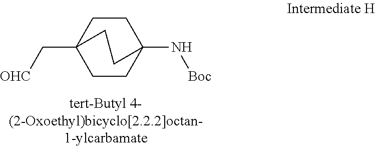 [SMILES: CC(C)(C)OC(=O)NC12CCC(CC=O)(CC1)CC2]

Step 1

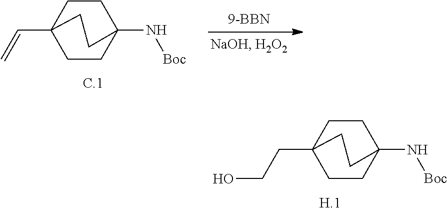 [SMILES: C=CC12CCC(NC(=O)OC(C)(C)C)(CC1)CC2 ; CC(C)(C)OC(=O)NC12CCC(CCO)(CC1)CC2]

To a solution of C.1 (5.00 g) in tetrahydrofuran (86 mL) was added a solution of 9-borabicyclo(3.3.1)nonane dimer (95.5 mL, 0.5 M in tetrahydrofuran) under cooling with ice, the mixture was stirred at the same temperature for 1 hour and further stirred at room temperature for 2 hours. After quenching the reaction by adding 3 M sodium hydroxide solution (19.9 mL) under cooling with ice, the mixture was added 30% hydrogen peroxide solution (26.5 mL) and stirred at the same temperature for 1 hour. After dilution of the mixture with dichloromethane, the mixture was washed with water and brine, dried over anhydrous sodium sulfate, filtered, and then concentrated in vacuo. Flash chromatography (silica, chloroform:methanol=10:1) of the residue gave H.1 (4.92 g).

¹H NMR (CDCl₃): δ 1.15 (br, 1H), 1.42 (s, 9H), 1.46-1.55 (m, 6H), 1.62-1.94 (m, 6H), 3.64 (d, J=7.3 Hz, 2H), 4.30 (br, 1H).

MS (CI⁺) m/z: 270 (MH⁺).

HRMS (CI⁺) for C₁₅H₂₈NO₃ (MH⁺): calcd, 270.2069. found, 270.2108.

Step 2

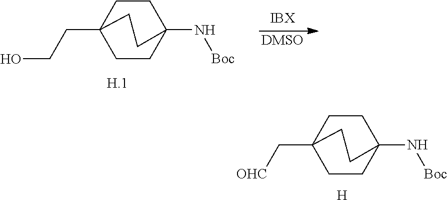

Compound H (963 mg) was prepared from H.1 (1.00 mg) in the same manner as described for the synthesis of A.

¹H NMR (CDCl₃): δ 1.42 (s, 9H), 1.61-1.72 (m, 6H), 1.80-2.18 (m, 6H), 2.18 (d, J=3.1 Hz, 2H), 4.31 (br, 1H), 9.79 (t, J=3.1 Hz, 1H).

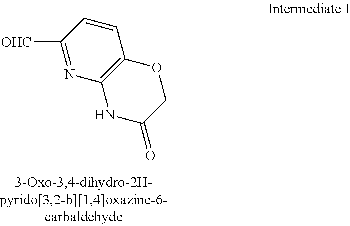 [SMILES: O=Cc1ccc2c(n1)NC(=O)CO2]

Step 1

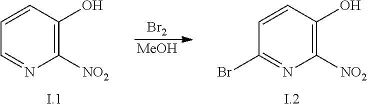 [SMILES: "MeOH" ; O=[N+]([O-])c1ncccc1O ; "Br2" ; O=[N+]([O-])c1nc(Br)ccc1O]

To a solution of 1.1 (140 g) in methanol (2.5 L) was added a solution of sodium methoxide [prepared from sodium (24.2 g) and methanol (215 mL)] at room temperature. The mixture was stirred at the same temperature for 30 minutes. Bromine (51.4 mL) was added dropwise to the mixture at 0° C., the mixture was stirred at the same temperature for 2 hours. After quenching the reaction by adding acetic acid (18 mL), the mixture was concentrated in vacuo to give I.2, which was used for the next step without further purification.

Step 2

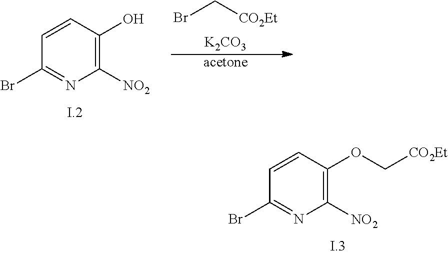

To a suspension of the crude 1.2 and potassium carbonate (277 g) in acetone (1.4 L) was added ethyl bromoacetate (111 mL), the mixture was heated at reflux for 8 hours. After dilution of the mixture with methyl tert-butyl ether (1.4 L), the resulting precipitates were filtered off. The filtrate was concentrated in vacuo to give 1.3, which was used for the next step without further purification.

Step 3

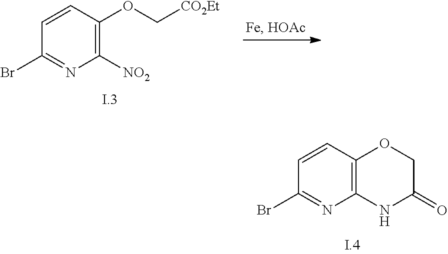 [SMILES: CCOC(=O)COc1ccc(Br)nc1[N+](=O)[O-] ; O=C1COc2ccc(Br)nc2N1]

A suspension of the crude 1.3 and iron powder (162 g) in acetic acid (1.2 L) was heated at 90° C. for 1.5 hours. After dilution of the mixture with ethyl acetate (2.4 L), the resulting precipitates were filtered off. The filtrate was concentrated in vacuo. Flash chromatography (hexane:ethyl acetate=2:1) of the residue gave 1.4 (69.0 g).

¹H NMR (CDCl₃): δ 4.67 (s, 2H), 7.10 (d, J=8.8 Hz, 1H), 7.14 (d, J=8.8 Hz, 1H), 8.01 (brs, 1H).

Step 4

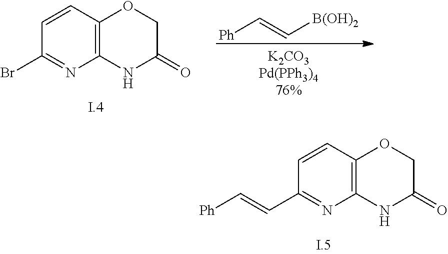 [SMILES: O=C1COc2ccc(Br)nc2N1 ; OB(O)/C=C/c1ccccc1 ; O=C1COc2ccc(/C=C/c3ccccc3)nc2N1]

To a degassed solution of I.4 (28.9 g) in 1,4-dioxane (630 mL) and water (100 mL) was added phenylvinylboronic acid (19.2 g), potassium carbonate (35.6 g) and tetrakis(triphenylphosphine)palladium (4.42 g), the mixture was heated at reflux for 24 hours. After dilution of the mixture with water (720 mL), the resulting precipitates were collected by filtration and washed with water (180 mL). Flash chromatography (NH silica gel, hexane:1,4-dioxane=2:1) of the crude product gave 1.5 (24.3 g).

¹H NMR (CDCl₃): δ 4.68 (s, 2H), 7.01 (d, J=7.9 Hz, 1H), 7.03 (d, J=15.9 Hz, 1H), 7.23 (d, J=7.9 Hz, 1H), 7.36 (t, J=7.3 Hz, 2H), 7.46 (d, J=15.9 Hz, 1H), 7.53 (d, J=7.3 Hz, 1H), 8.09 (brs, 1H).

Step 5

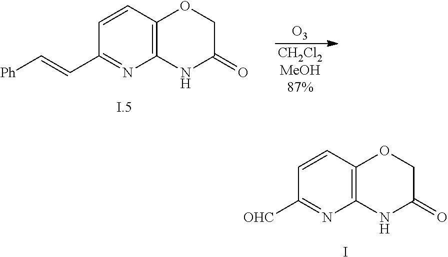

A suspension of I.5 (24.0 g) in dichloromethane (1.2 L) and methanol (420 mL) was bubbled with ozone at −71° C. until a pale blue color appeared. The excess ozone was removed by bubbling air through the suspension for 30 minutes. Dimethyl sulfide (36 mL) was added to the suspension. The mixture was stirred at room temperature for overnight and concentrated in vacuo. After dilution of the mixture with diethyl ether (130 mL) and 0.5 M hydrochloric acid (65 mL), the resulting precipitates were collected by filtration and washed with water (40 mL×3) and diethyl ether (40 mL). Treatment of the crude product with acetone (80 mL) gave I (14.7 g).

¹H NMR (CDCl₃): δ 4.80 (s, 2H), 7.39 (d, J=7.9 Hz, 1H), 7.69 (d, J=7.9 Hz, 1H), 8.35 (brs, 1H), 9.89 (s, 1H).

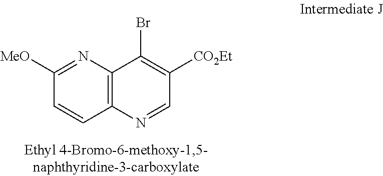 [SMILES: CCOC(=O)c1cnc2ccc(OC)nc2c1Br]

Step 1

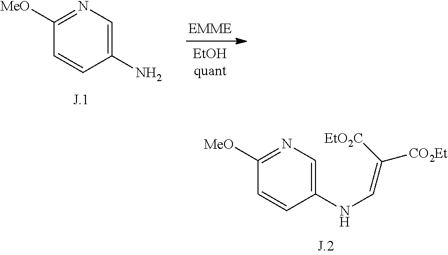 [SMILES: COc1ccc(N)cn1 ; CCOC(=O)C(=CNc1ccc(OC)nc1)C(=O)OCC]

A mixture of J.1 (100 g) and diethyl ethoxymethylenemalonate (178 g) in ethanol (1 L) was heated under reflux for 2 hours. The mixture was concentrated in vacuo to give J.2 (244 g).

¹H NMR (CDCl₃): δ 1.32 (t, J=7.4 Hz, 3H), 1.38 (t, J=7.4 Hz, 3H), 3.94 (s, 3H), 4.24 (q, J=7.4 Hz, 2H), 4.31 (q, J=7.4 Hz, 2H), 6.78 (d, J=8.6 Hz, 1H), 7.43 (dd, J=9.2, 3.1 Hz, 1H), 8.03 (d, J=3.1 Hz, 1H), 8.37 (d, J=3.1 Hz, 1H), 10.90-11.10 (m, 1H).

Step 2

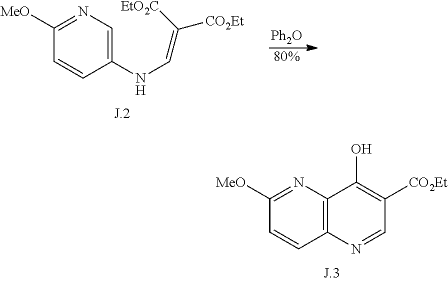

J.2 (60.0 g) was added portionwise to diphenyl ether (300 mL) at 260° C. for 5 minutes. After cooling, the mixture was diluted with pentane. The resulting precipitates were collected by filtration and washed with hexane to give crude J.3. Another two experiments at the same reaction scale gave the crude product J.3. The combined crude J.3 was stirred in hexane (1.2 L), the precipitates were collected by filtration and washed with hexane to give J.3 (157.2 g).

¹H NMR (DMSO-d₆): δ 1.27 (t, J=6.7 Hz, 3H), 3.94 (s, 3H), 4.21 (t, J=6.7 Hz, 2H), 7.20 (d, J=8.6 Hz, 1H), 7.99 (d, J=9.2 Hz, 1H), 8.49 (brs, 1H).

Step 3

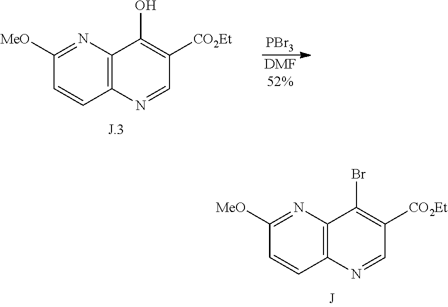 [SMILES: CCOC(=O)c1cnc2ccc(OC)nc2c1O ; "PBr3" ; "DMF52%" ; CCOC(=O)c1cnc2ccc(OC)nc2c1Br]

To a suspension of J.3 (312 g) in anhydrous N,N-dimethylformamide (1.1 L) was added phosphorous tribromide (175 mL) under cooling with water, the mixture was stirred at room temperature for 2.5 hours. The mixture was poured into ice water (4 L), the mixture was adjusted to pH 8 by addition of saturated sodium hydrogencarbonate solution. The resulting precipitates were collected by filtration, washed with water, and dried. Flash chromatography (toluene:ethyl acetate=5:1) of the crude product gave J (203 g).

¹H NMR (DMSO-d₆): δ 1.37 (t, J=7.3 Hz, 3H), 4.09 (s, 3H), 4.43 (q, J=7.3 Hz, 2H), 7.43 (d, J=9.1 Hz, 1H), 8.36 (d, J=9.1 Hz, 1H), 8.91 (s, 1H).

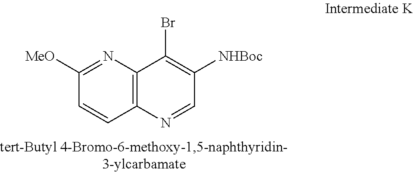 [SMILES: COc1ccc2ncc(NC(=O)OC(C)(C)C)c(Br)c2n1]

Step 1

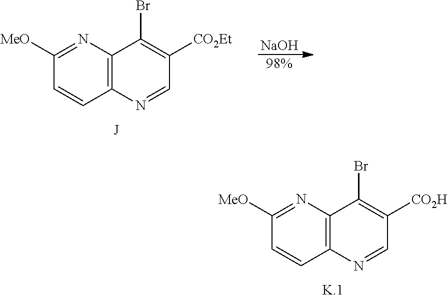 [SMILES: CCOC(=O)c1cnc2ccc(OC)nc2c1Br ; "NaOH" ; "98%" ; COc1ccc2ncc(C(=O)O)c(Br)c2n1]

A suspension of J (192 g) in tetrahydrofuran (1.9 L) was added 2 N sodium hydroxide solution (694 mL) under cooling with ice, the mixture was stirred at room temperature for 3 hours. After quenching the reaction by adding of 2 N hydrochloric acid (375 mL, pH 6), the mixture was evaporated in vacuo to remove tetrahydrofuran. The aqueous mixture was adjusted to pH 2 by addition of 2 N hydrochloric acid (400 mL) and diluted with water (1.3 L). The resulting precipitates were collected by filtration and washed with water to give K.1 (171 g).

¹H NMR (DMSO-d₆): δ 4.09 (s, 3H), 7.41 (d, J=9.1 Hz, 1H), 8.35 (d, J=8.5 Hz, 1H), 14.03 (s, 1H).

Step 2

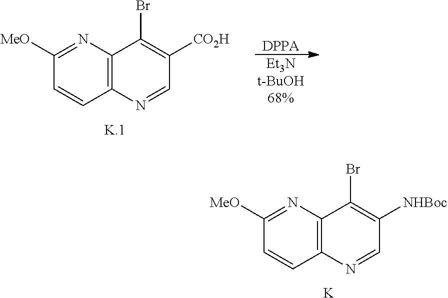

A mixture of K.1 (169 g), diphenyl phosphoryl azide (141 mL), triethylamine (744 mL) and anhydrous tert-butanol (886 mL) in anhydrous N,N-dimethylformamide (2 L) was heated at 100° C. for 1 hour and concentrated in vacuo. After dilution of the residue with ethyl acetate, the mixture was washed with saturated sodium hydrogencarbonate solution and brine, dried over anhydrous sodium sulfate, filtered, and then concentrated in vacuo. Flash chromatography (hexane:ethyl acetate=3:1) of the residue gave K (144 g).

¹H NMR (DMSO-d₆): δ 1.49 (s, 9H), 4.06 (s, 3H), 7.26 (d, J=9.2 Hz, 1H), 8.29 (d, J=9.2 Hz, 1H), 8.83 (s, 1H), 9.15 (s, 1H).

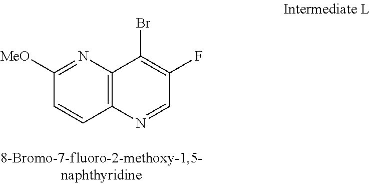 [SMILES: COc1ccc2ncc(F)c(Br)c2n1]

Step 1

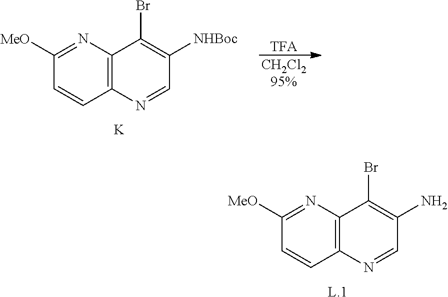

To a solution of K (98.0 g) in dichloromethane (280 mL) was added trifluoroacetic acid (166 mL) at −10° C., the mixture was stirred at room temperature for overnight and concentrated in vacuo. After dilution of the residue with chloroform, the mixture was poured onto saturated sodium hydrogencarbonate solution (2.3 L, pH 8). The resulting precipitates were collected by filtration and washed with water to give L.1 (54.0 g). The combined mixture of the filtrate and washing was extracted with chloroform (1 L). The organic extracts were dried over anhydrous sodium sulfate, filtered, and then concentrated in vacuo to give L.1 (total 67.1 g).

¹H NMR (DMSO-d₆): δ 4.00 (s, 3H), 6.21 (brs, 2H), 6.88 (d, J=8.6 Hz, 1H), 8.05 (d, J=8.6 Hz, 1H), 8.34 (s, 1H).

Step 2

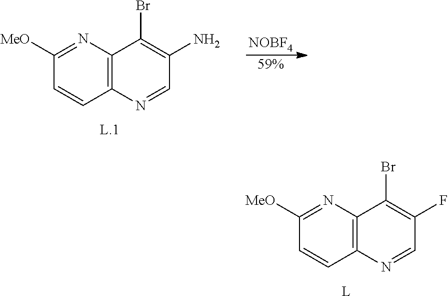

To a solution of L.1 (37.1 g) in anhydrous tetrahydrofuran (580 mL) was added nitrosyl tetrafluoroborate (20.8 g) at −10° C., the mixture was stirred at the same temperature for 50 minutes. Additional nitrosyl tetrafluoroborate (5.39 g) was added to the mixture at the same temperature. After stirring for 35 minutes, additional nitrosyl tetrafluoroborate (1.80 g) was added to the mixture. After stirring for 5 minutes, the resulting precipitates were collected by filtration and washed with cold tetrahydrofuran to give diazonium salt as yellow solid (49.1 g). A suspension of the salt (49.1 g) in decaline (730 mL) was heated at 100° C. for 1 hour. After cooling with NaCl-ice bath, the precipitates were collected by filtration and dissolved with ethyl acetate. The mixture was washed with saturated sodium hydrogencarbonate solution, dried over anhydrous sodium sulfate, filtered, and then concentrated in vacuo. Flash chromatography (toluene:ethyl acetate=30:1) of the residue gave L (22.0 g).

¹H NMR (DMSO-d₆): δ 4.09 (s, 3H), 7.32 (d, J=9.2 Hz), 8.36 (d, J=9.2 Hz), 8.87 (s, 1H).

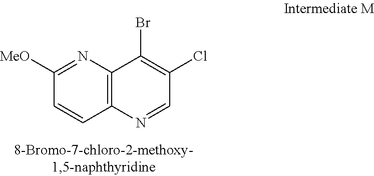 [SMILES: COc1ccc2ncc(Cl)c(Br)c2n1]

Step 1

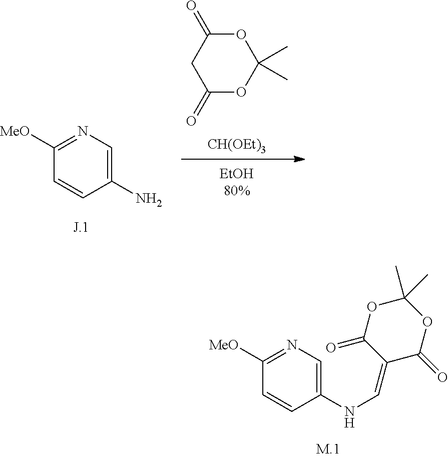

A mixture of J.1 (87.9 g), Meldrum's acid (120 g) and triethyl orthoformate (105 mL) in ethanol (527 mL) was heated under reflux for 1 hour. The resulting precipitates were collected by filtration and washed with ethanol to give M.1 (157 g).

¹H NMR (CDCl₃): δ 1.76 (s, 6H), 3.96 (s, 3H), 6.83 (d, J=8.6 Hz, 1H), 7.52 (dd, J=8.6, 3.1 Hz, 1H), 8.12 (d, J=3.1 Hz, 1H), 8.49 (d, J=14.1 Hz, 1H), 11.18 (d, J=14.1 Hz, 1H).

Step 2

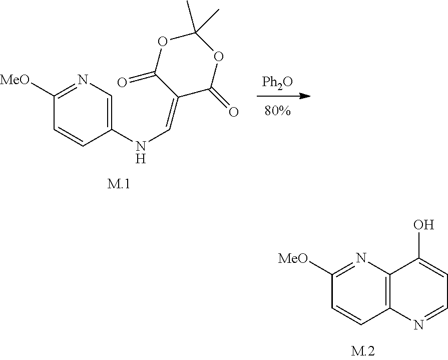

M.1 (54.0 g) was added portionwise to Dowtherm A (320 mL) (Sigma-Aldrich, St. Louis, Mo.) at 240° C. for 5 minutes. After cooling, the resulting precipitates were collected by filtration and washed with diethyl ether to give M.2 (27.3 g).

¹H NMR (DMSO-d₆): δ 3.93 (s, 3H), 6.23 (brs, 1H), 7.15 (d, J=8.6 Hz, 1H), 7.94 (d, J=8.6 Hz, 1H), 8.65 (d, J=2.4 Hz, 1H), 11.72 (brs, 1H).

Step 3

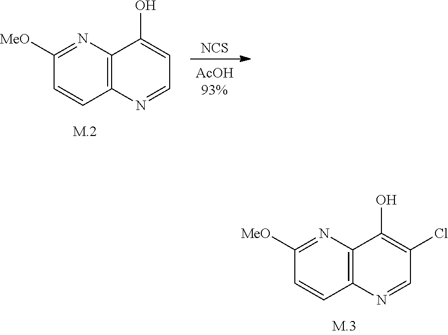

To a solution of M.2 (50.0 g) in acetic acid (heating was needed to dissolve) was added N-chlorosuccinimide (41.7 g), the mixture was stirred at 35-40° C. for 4 hours. The resulting precipitates were collected by filtration. A suspension of the crude product in water was stirred at 80° C. for 1 hour. The precipitates were collected by filtration and washed with water to give M.3 (55.4 g).

¹H NMR (DMSO-d₆): δ 4.07 (s, 3H), 7.47 (d, J=9.2 Hz, 1H), 8.45 (d, J=9.2 Hz, 1H), 8.65 (d, J=2.4 Hz, 1H), 9.08 (s, 1H).

Step 4

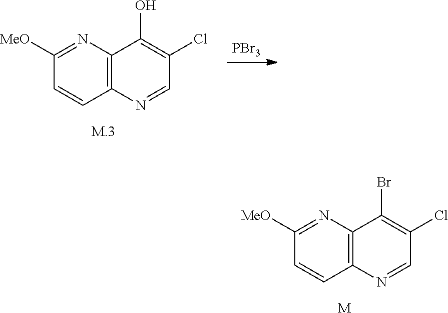

To a solution of M.3 (27 g) in N,N-dimethylformamide (408 mL) was added dropwise phosphorous tribromide (16.4 mL) at 0° C., the mixture was stirred at the same temperature and stirred at room temperature for 2 hours. After dilution of the mixture with ethyl acetate, the mixture was washed with saturated sodium hydrogencarbonate solution. The resulting precipitates were collected by filtration to give the crude M (19.6 g). The organic extracts of the filtrate were dried over anhydrous sodium sulfate, filtered, and then concentrated in vacuo to give the crude M (15.9 g). Recrystallization of the combined crude M from ethanol gave M (25.5 g).

¹H NMR (CDCl₃): δ 4.16 (s, 3H), 7.15 (d, J=8.6 Hz, 1H), 8.19 (d, J=8.6 Hz, 1H), 8.69 (s, 1H).

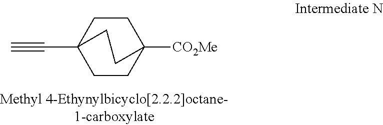

To a solution of dimethyl 1-diazo-2-oxopropylphosphonate (4.41 g) in dichloromethane (70 mL) was added potassium carbonate (1.69 g) and a solution of methyl 4-formylbicyclo[2.2.2]octane-1-carboxylate (1.50 g) in methanol (70 mL) under cooling with ice, the mixture was stirred at room temperature for 1 hour. After quenching the reaction by adding saturated ammonium chloride solution under cooling with ice, the organic extracts were washed with saturated ammonium chloride solution and water, dried over anhydrous sodium sulfate, filtered, and then concentrated in vacuo. Flash chromatography (silica, hexane:dichloromethane=1:1) of the residue gave N (998 mg).

¹H NMR (CDCl₃): δ 1.80 (s, 12H), 2.09 (s, 1H), 3.64 (s, 3H).

MS (CI⁺) m/z: 193 (MH⁺).

HRMS (CI⁺) for C₁₂H₁₇O₂ (MH⁺): calcd, 193.1229. found, 193.1244.

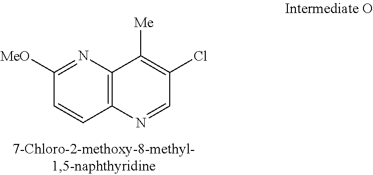 [SMILES: COc1ccc2ncc(Cl)c(C)c2n1]

Step 1

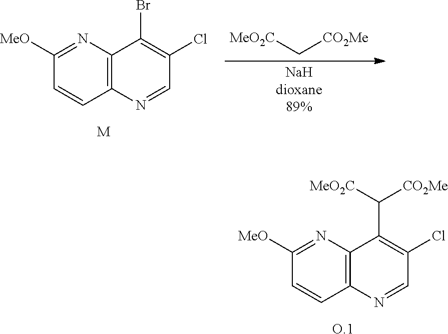

To a suspension of sodium hydride (4.02 g, 60% in mineral oil) in anhydrous 1,4-dioxane (110 mL) was added dimethyl malonate (12.5 mL) under cooling with ice, the mixture was heated at 75° C. for 2 hours. The resulting suspension was added 4-bromo-3-chloronaphthyridine M (10.00 g) and copper bromide (CuBr, 1.84 g), the mixture was heated at 100° C. for 18 hours. After quenching the reaction by adding 2 M hydrochloric acid (50 mL, pH 3), the mixture was diluted with ethyl acetate. The insoluble materials were filtered off, the filtrate was washed with saturated sodium hydrogencarbonate solution and brine. The organic extracts were washed with brine, dried over anhydrous sodium sulfate, filtered, and then concentrated in vacuo. Flash chromatography (hexane:ethyl acetate=3:1) of the residue gave O.1 (10.52 g).

¹H NMR (CDCl₃): δ 3.74 (s, 6H), 3.99 (s, 3H), 5.80 (s, 1H), 7.12 (d, J=9.2 Hz, 2H), 8.20 (d, J=8.6 Hz, 2H), 8.76 (s, 1H).

Step 2

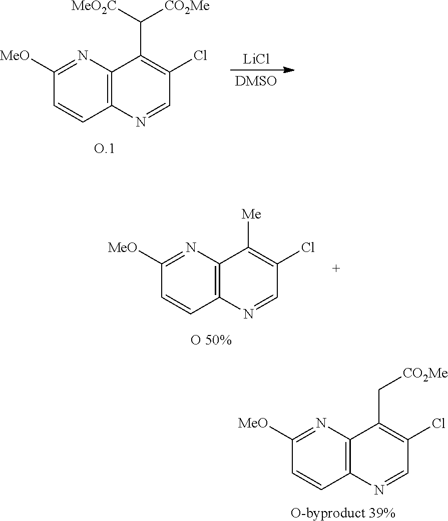

A mixture of O.1 (4.00 g), lithium chloride (2.61 g) and water (560 uL) was heated at 120° C. for overnight. After dilution of the mixture with water, the mixture was extracted with ethyl acetate. The organic extracts were washed with water and brine, dried over anhydrous sodium sulfate, filtered, and then concentrated in vacuo. Flash chromatography (hexane:ethyl acetate=5:1) of the residue gave O (1.29 g, less polar) and O-byproduct (1.26 g, more polar).

¹H NMR (CDCl₃): δ 2.78 (s, 6H), 4.10 (s, 3H), 7.10 (d, J=9.2 Hz, 1H), 8.16 (d, J=9.2 Hz, 1H), 8.66 (s, 1H).

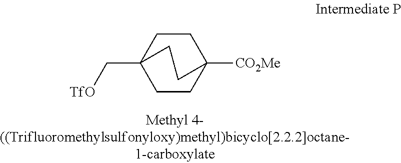 [SMILES: COC(=O)C12CCC(COS(=O)(=O)C(F)(F)F)(CC1)CC2]

To a solution of methyl 4-(hydroxymethyl)bicyclo[2.2.2]octane-1-carboxylate (2.70 g, prepared according to International Patent Application Publication No. WO 2001034610) and 2,6-lutidine (2.54 mL) in dichloromethane (55 mL) was added triflic anhydride (2.97 mL) under cooling with ice, the mixture was stirred at the same temperature for 1.5 hours. After dilution of the mixture with water, the mixture was extracted with dichloromethane. The organic extracts were washed with aqueous sodium hydrogencarbonate solution, 10% hydrochloric acid and brine, dried over anhydrous sodium sulfate, filtered, and then concentrated in vacuo. Flash chromatography (silica, hexane:ethyl acetate=6:1) of the residue gave P (4.38 g).

¹H NMR (CDCl₃): δ 1.51-1.58 (m, 6H), 1.82-1.85 (m, 6H), 3.66 (s, 3H), 4.17 (s, 2H).

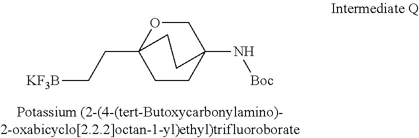

To a solution of B (650 mg) in tetrahydrofuran (3.2 mL) was added a solution of 9-borabicyclo(3.3.1)nonane dimer (12.3 mL, 0.5 M in tetrahydrofuran) under cooling with ice, the mixture was stirred at the same temperature for 5 hours. After quenching the reaction by adding water (3.4 mL) under cooling with ice, the mixture was added a solution of formaldehyde (830 μL, 37 wt % in water), and the mixture was stirred at room temperature for overnight. After dilution of the mixture with brine, the mixture was extracted with ethyl acetate. The organic extracts were concentrated in vacuo. A solution of the residue in acetone (21 mL) and water (1.7 mL) was added potassium hydrogenfluoride (2.00 g) under cooling with ice, the mixture was stirred at room temperature for 4 hours, and then concentrated in vacuo. After washing the residue with diethyl ether, the insoluble materials were extracted with acetone by Soxhlet extractor to give Q (823 mg).

¹H NMR (DMSO-d₆): δ 0.94-1.13 (m, 6H), 1.21-1.59 (m, 4H), 1.68 (s, 9H), 1.81-1.88 (m, 2H), 3.67 (s, 2H).

MS (FAB⁺) m/z: 362 (MH⁺).

HRMS (FAB⁺) for C₁₄H₂₅BF₃KNO₃ (MH⁺): calcd, 362.1517. found, 362.1528.

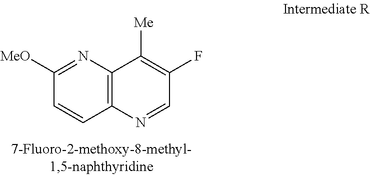 [SMILES: COc1ccc2ncc(F)c(C)c2n1]

A degassed mixture of L (15.0 g), methylboronic acid (6.99 g), tetrakis(triphenylphosphine)palladium (6.74 g), saturated potassium carbonate solution (45.6 mL) and 1,4-dioxane (70.7 mL) was stirred at 100° C. for 100 hours, and then concentrated in vacuo. After dilution of the residue with ethyl acetate, the mixture was washed with water and brine, dried over anhydrous sodium sulfate, filtered, and then concentrated in vacuo. Flash chromatography (silica, hexane:ethyl acetate=4:1) of the residue gave R (9.25 g).

¹H NMR (DMSO-d₆): δ 2.64 (d, J=1.8 Hz, 3H), 4.10 (s, 3H), 7.07 (d, J=9.2 Hz, 1H), 8.17 (d, J=9.2 Hz, 1H), 8.61 (s, 1H).

MS (EI⁺) m/z: 192 (M⁺).

HRMS (EI⁺) for C₁₀H₉FN₂O (M⁺): calcd, 192.0699. found, 192.0715.

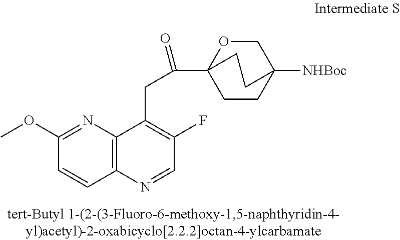 [SMILES: COc1ccc2ncc(F)c(CC(=O)C34CCC(NC(=O)OC(C)(C)C)(CC3)CO4)c2n1]

To a solution of tert-butyl 1-(2-(3-fluoro-6-methoxy-1,5-naphthyridin-4-yl)-1-hydroxyethyl)-2-oxabicyclo[2.2.2]octan-4-ylcarbamate (300 mg) in dichloromethane (6.7 mL) was added Dess-Martin periodinane (313 mg) at room temperature, the mixture was stirred at the same temperature for 18 hours. The mixture was washed with saturated sodium hydrogencarbonate solution, saturated sodium sulfite solution and brine. The organic extracts were dried over anhydrous sodium sulfate, filtered, and then concentrated in vacuo. Flash chromatography (silica, toluene:ethyl acetate=2:1) of the residue gave tert-butyl 1(2-(3-fluoro-6-methoxy-1,5-naphthyridin-4-yl)acetyl)-2-oxabicyclo[2.2.2]octan-4-ylcarbamate (264 mg).

¹H NMR (CDCl₃): δ 1.83-1.90 (m, 2H), 1.99-2.10 (m, 2H), 2.11-2.24 (m, 4H), 4.01 (s, 3H), 4.15 (s, 2H), 4.35 (brs, 1H), 4.54 (s, 2H), 7.05 (d, J=8.6 Hz, 1H), 8.17 (d, J=8.6 Hz, 1H), 8.65 (s, 1H).

MS (ESI⁺) m/z: 446 (MH⁺)

HRMS (ESI⁺) for C₂₃H₂₉FN₃O₅ (MH⁺): calcd, 446.20912. found, 446.20918.

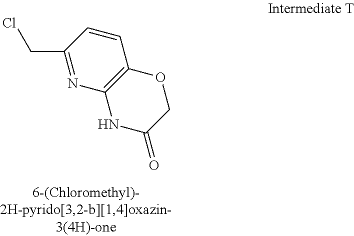 [SMILES: O=C1COc2ccc(CCl)nc2N1]

Step 1

A suspension of I (3.00 g) and 10% Pd—C (300 mg) in methanol (60 mL) and dichloromethane (18 mL) was stirred at room temperature for 7 hours under H₂ atmosphere (1 kg/cm²). After the insoluble materials were filtered off, the filtrate was concentrated in vacuo to give 6-(hydroxymethyl)-2H-pyrido[3,2-b][1,4]oxazin-3(4H)-one (3.00 g).

¹H NMR (DMSO-d₆): δ 4.41 (d, J=5.5 Hz, 2H), 4.60 (s, 2H), 5.31 (t, J=5.8 Hz, 1H), 7.01 (d, J=8.6 Hz, 1H), 7.32 (d, J=7.9 Hz, 1H), 11.16 (s, 1H).

MS (EI⁺) m/z: 180 (M⁺).

HRMS (EI⁺) for C₈H₈N₂O₃ (M⁺): calcd, 180.0535. found, 180.0517.

Step 2

To a suspension of 6-(hydroxymethyl)-2H-pyrido[3,2-b][1,4]oxazin-3(4H)-one (3.31 g), lithium chloride (3.90 g) and triethylamine (3.30 mL) was added methanesulfonyl chloride (1.70 mL) under cooling with ice, the mixture was stirred at room temperature for 22 hours. The mixture was washed with water and brine. The organic extracts were dried over anhydrous sodium sulfate, filtered, and then concentrated in vacuo. Treatment of the residue with diisopropyl ether gave 6-(chloromethyl)-2H-pyrido[3,2-b][1,4]oxazin-3(4H)-one (3.26 g).

¹H NMR (CDCl₃): δ 4.60 (s, 2H), 4.68 (s, 2H), 7.08 (d, J=7.9 Hz, 1H), 7.26 (d, J=7.9 Hz, 1H), 8.67 (s, 1H).

MS (EI⁺) m/z: 198 (M⁺).

HRMS (EI⁺) for C₈H₇ClN₂O₂ (M⁺): calcd, 198.0196. found, 198.0229.

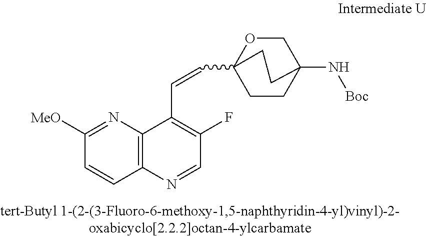 [SMILES: COc1ccc2ncc(F)c(C=CC34CCC(NC(=O)OC(C)(C)C)(CC3)CO4)c2n1]

See Step 1 of EXAMPLE 18

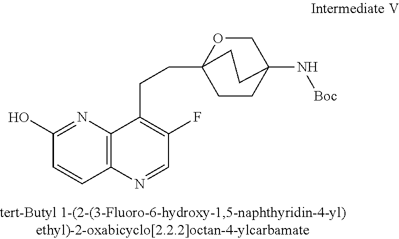 [SMILES: CC(C)(C)OC(=O)NC12CCC(CCc3c(F)cnc4ccc(O)nc34)(CC1)OC2]

A solution of tert-butyl 1-(2-(3-fluoro-6-methoxy-1,5-naphthyridin-4-yl)ethyl)-2-oxabicyclo[2.2.2]octan-4-ylcarbamate (3.50 g) in 1,4-dioxane (42 mL) and 6 N hydrochloric acid (42 mL) was stirred at 70° C. for 30 hours. The mixture was concentrated in vacuo to give 8-(2-(4-amino-2-oxabicyclo[2.2.2]octan-1-yl)ethyl)-7-fluoro-1,5-naphthyridin-2-ol hydrochloride (3.04 g). To a mixture of the obtained hydrochloride (2.87 g), tetrahydrofuran and saturated sodium hydrogencarbonate solution (41 mL) was added di-tert-butyl dicarbonate (1.77 g), the mixture was stirred at 60° C. for overnight. After dilution of the mixture with water, the mixture was extracted with dichloromethane. The organic extracts were washed with brine, dried over anhydrous sodium sulfate, filtered, and then concentrated in vacuo. Flash chromatography (silica, dichloromethane:acetone=5:1) of the residue gave tert-butyl 1-(2-(3-fluoro-6-hydroxy-1,5-naphthyridin-4-yl)ethyl)-2-oxabicyclo[2.2.2]octan-4-ylcarbamate (2.20 g).

¹H NMR (DMSO-d₆): δ 1.36 (s, 9H), 1.46-2.00 (m, 10H), 2.85-2.96 (m, 2H), 3.78 (s, 2H), 6.59 (s, 1H), 6.69 (d, J=9.8 Hz, 1H), 7.90 (d, J=9.8 Hz, 1H), 8.40 (s, 1H).

MS (ESI⁺) m/z: 418 (MH⁺).

HRMS (ESI⁺) for C₂₂H₂₉FN₃O₄ (MH⁺): calcd, 418.21421. found, 418.21404.

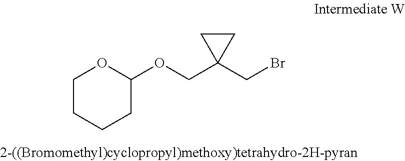 [SMILES: BrCC1(COC2CCCCO2)CC1]

The title compound 2-((1-(bromomethyl)cyclopropyl)methoxy)tetrahydro-2H-pyran (216 mg) was prepared from (1-((tetrahydro-2H-pyran-2-yloxy)methyl)cyclopropyl)methanol (310 mg, prepared according to methods described in Arai et al., 1983, Journal of Medicinal Chemistry. 26:72-78.) in the same manner as described for the synthesis of step 2 of X.

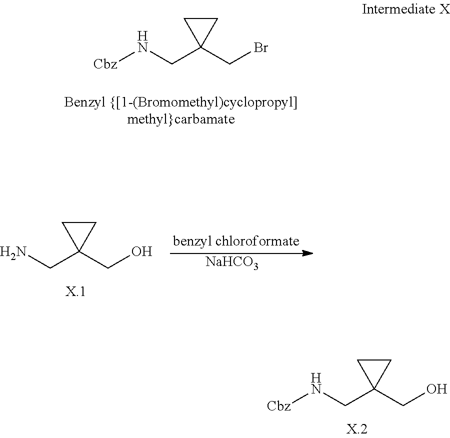

Step 1

To a mixture of X.1 (50.0 mg), sodium hydrogencarbonate (125 mg) in water/tetrahydrofuran (1 mL, 1:1) was added benzyl chloroformate (95 uL) under cooling with ice, the mixture was stirred at room temperature for 3 hours. The mixture was extracted with ethyl acetate. The organic extracts were washed with brine, dried over anhydrous sodium sulfate, filtered, and then concentrated in vacuo. Flash chromatography (silica, hexane:ethyl acetate=2:1) of the residue gave X.2 (80.1 mg).

¹H NMR (CDCl₃): δ 0.46 (s, 4H), 2.78 (br, 1H), 3.20 (d, J=6.1 Hz, 2H), 3.41 (s, 2H), 5.12 (s, 2H), 5.20 (br, 1H), 7.29-7.39 (m, 5H).

MS (CI⁺) m/z: 236 (MH⁺).

HRMS (CI⁺) for C₁₃H₁₈NO₃ (MH⁺): calcd, 236.1287. found, 236.1298.

Step 2

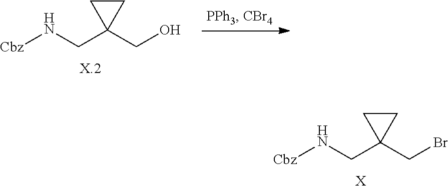

To a solution of X.2 (80.1 mg) and triphenylphosphine (114 mg) in dichloromethane (1.9 mL) was added carbon tetrabromide (144 mg) under cooling with ice, the mixture was stirred at room temperature for 2 hours. The mixture was washed with water. The organic extracts were washed with brine, dried over anhydrous sodium sulfate, filtered, and then concentrated in vacuo. Flash chromatography (silica, hexane:ethyl acetate=7:1) of the residue gave X (70.5 mg).

¹H NMR (CDCl₃): δ 0.55-0.68 (m, 2H), 0.83 (brs, 2H), 3.30 (d, J=6.1 Hz, 2H), 3.40 (s, 2H), 4.98 (br, 1H), 5.11 (s, 2H), 7.29-7.41 (m, 5H).

MS (EI⁺) m/z: 297 (M⁺).

HRMS (EI⁺) for C₁₃H₁₆BrNO₂ (M⁺): calcd, 297.0364. found, 297.0401.

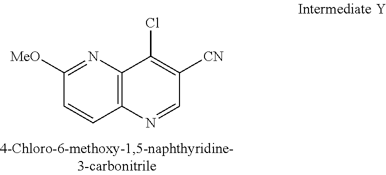 [SMILES: COc1ccc2ncc(C#N)c(Cl)c2n1]

Step 1

To a suspension of K.1 (1.00 g) in toluene (12 mL) was added thionyl chloride (3.5 mL), the mixture was stirred at reflux for 3 hours, and then concentrated in vacuo to give acid chloride. To a suspension of the crude acid chloride in dichloromethane (4 mL) was added concentrated ammonium hydroxide (8 mL) under cooling with ice, the mixture was stirred at room temperature for 1 hour, and then concentrated in vacuo. Treatment of the residue with water gave 4-chloro-6-methoxy-1,5-naphthyridine-3-carboxamide (822 mg).

¹H NMR (CDCl₃): δ 4.15 (s, 3H), 6.12 (brs, 1H), 6.63 (brs, 1H), 7.24 (d, J=9.2 Hz, 1H), 8.24 (d, J=9.2 Hz, 1H), 9.09 (s, 1H).

MS (EI⁺) m/z: 237 (M⁺).

HRMS (EI⁺) for C₁₀H₈ClN₃O₂ (M⁺): calcd, 237.0305. found, 237.0289.

Step 2

To a suspension of 4-chloro-6-methoxy-1,5-naphthyridine-3-carboxamide (800 mg) in dichloromethane (3.0 mL) was added triethylamine (2.5 mL) and trifluoroacetic anhydride (1.3 mL) under cooling with ice, the mixture was stirred at the room temperature for 2 hours. The mixture was diluted with dichloromethane, washed with water. The organic extracts were dried over anhydrous sodium sulfate, filtered, and then concentrated in vacuo. Treatment of the residue with ethanol gave Y (662.6 mg).

¹H NMR (CDCl₃): δ 4.17 (s, 3H), 7.30 (d, J=9.2 Hz, 1H), 8.25 (d, J=9.2 Hz, 1H), 8.85 (s, 1H).

MS (EI⁺) m/z: 219 (M⁺).

HRMS (EI⁺) for C₁₀H₆ClN₃O (M⁺): calcd, 219.0199. found, 219.0203.

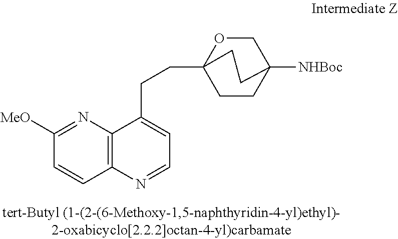 [SMILES: COc1ccc2nccc(CCC34CCC(NC(=O)OC(C)(C)C)(CC3)CO4)c2n1]

See Step 1 of EXAMPLE 17

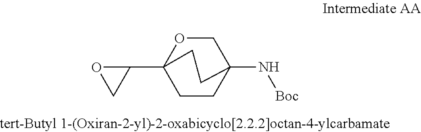 [SMILES: CC(C)(C)OC(=O)NC12CCC(C3CO3)(CC1)OC2]

To a solution of AB (81.0 mg) in methanol (1.2 mL) was added potassium carbonate (25.4 mg) under cooling with ice, the mixture was stirred at the same temperature for 6 hours and further stirred at room temperature for overnight. After dilution of the mixture with water, the mixture was extracted with ethyl acetate. The organic extracts were washed with brine, dried over anhydrous sodium sulfate, filtered, and then concentrated in vacuo. Flash chromatography (silica, hexane:ethyl acetate=2:1) of the residue gave AA (46.9 mg).

¹H NMR (CDCl₃): δ 1.42 (s, 9H), 1.62-2.13 (m, 8H), 2.68-2.73 (m, 2H), 2.87 (m, 1H), 3.95 (s, 1H), 4.28 (brs, 1H).

MS (CI⁺) m/z: 270 (MH⁺).

HRMS (CI⁺) for C₁₄H₂₄NO₄ (MH⁺): calcd, 270.1705. found, 270.1710.

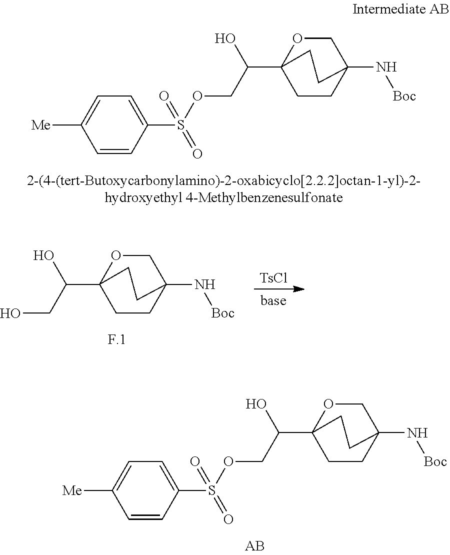 [SMILES: Cc1ccc(S(=O)(=O)OCC(O)C23CCC(NC(=O)OC(C)(C)C)(CC2)CO3)cc1 ; CC(C)(C)OC(=O)NC12CCC(C(O)CO)(CC1)OC2 ; "TsCl" ; "base" ; Cc1ccc(S(=O)(=O)OCC(O)C23CCC(NC(=O)OC(C)(C)C)(CC2)CO3)cc1]

To a solution of F.1 (2.00 g) and N,N,N′,N′-tetramethylpropanediamine (1.74 mL) in acetonitrile (63 mL) was added a solution of tosylchloride (1.46 g) in acetonitrile (7 mL) under cooling with ice, the mixture was stirred at the same temperature for 3 hours. After dilution of the mixture with water, the mixture was extracted with ethyl acetate. The organic extracts were washed with brine, dried over anhydrous sodium sulfate, filtered, and then concentrated in vacuo. Flash chromatography (silica, ethyl acetate) of the residue gave AB (1.78 g). Optical resolution (CHIRALPAK IA, methyl tert-butyl ether:isopropanol=92:8) of the racemate (508 mg) gave Enantiomer A (252 mg) and Enantiomer B (248 mg).

Enantiomer A: ¹H NMR (CDCl₃): δ 1.26 (br, 1H), 1.41 (s, 9H), 1.62-2.08 (m, 8H), 2.45 (m, 3H), 3.60-3.64 (m, 1H), 3.89-3.96 (m, 3H), 4.18 (dd, J=10.4, 3.1 Hz, 1H), 4.28 (brs, 1H), 7.35 (d, J=7.9 Hz, 2H), 7.79 (d, J=8.6 Hz, 2H).

Enantiomer B: ¹H NMR (CDCl₃): δ 1.27 (br, 1H), 1.41 (s, 9H), 1.62-2.09 (m, 8H), 2.47 (m, 3H), 3.62-3.66 (m, 1H), 3.91-3.98 (m, 3H), 4.19 (dd, J=10.4, 3.7 Hz, 1H), 4.29 (brs, 1H), 7.36 (d, J=8.0 Hz, 2H), 7.80 (d, J=8.0 Hz, 2H).

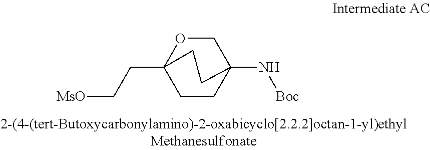 [SMILES: CC(C)(C)OC(=O)NC12CCC(CCOS(C)(=O)=O)(CC1)OC2]

Step 1

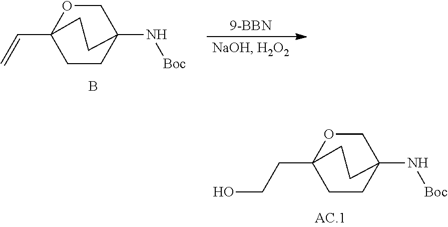

The compound AC.1 (4.62 g) was prepared from B (5.00 g). To a solution of B (5.00 g) in tetrahydrofuran (86 mL) was added a solution of 9-borabicyclo(3.3.1)nonane dimer (94.5 mL, 0.5 M in tetrahydrofuran) under cooling with ice, the mixture was stirred at room temperature for 2 hours. After quenching the reaction by adding 3 M sodium hydroxide solution (19.8 mL) under cooling with ice, the mixture was added 30% hydrogen peroxide solution (26.9 mL) and stirred at the same temperature for 1 hour. After dilution of the mixture with dichloromethane, the mixture was washed with water and brine, dried over anhydrous sodium sulfate, filtered, and then concentrated in vacuo. Flash chromatography (silica, hexane:acetone=2:1) of the residue gave AC.1 (4.62 g).

¹H NMR (CDCl₃): δ 1.42 (s, 9H), 1.59-2.14 (m, 10H), 3.11 (t, J=5.5 Hz, 1H), 3.75 (dd, J=10.1, 5.5 Hz, 2H), 3.94 (s, 1H), 4.26 (brs, 1H).

MS (CI⁺) m/z: 272 (MH⁺).

HRMS (CI⁺) for C₁₄H₂₆NO₄ (MH⁺): calcd, 272.1862. found, 272.1861.

Step 2

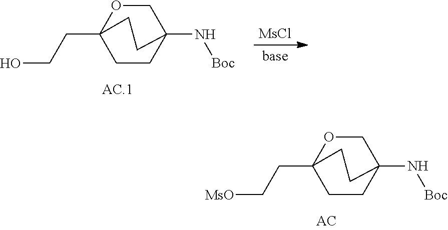 [SMILES: "MsCl" ; CC(C)(C)OC(=O)NC12CCC(CCO)(CC1)OC2 ; "base" ; CC(C)(C)OC(=O)NC12CCC(CCOS(C)(=O)=O)(CC1)OC2]

The compound AC (5.45 g) was prepared from AC.1 (4.50 g). To a solution of AC.1 (4.50 g) and triethylamine (3.46 mL) in dichloromethane (170 mL) was added methanesulfonyl chloride (1.54 mL) under cooling with ice, the mixture was stirred at the same temperature for 1.5 hours. After dilution of the mixture with ice water, the mixture was extracted with dichloromethane. The organic extracts were washed with brine, dried over anhydrous sodium sulfate, filtered, and then concentrated in vacuo. Flash chromatography (silica, hexane:ethyl acetate=1:1) of the residue gave AC (5.45 g).

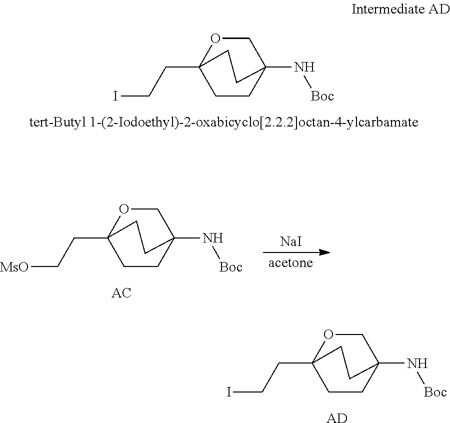

A mixture of AC (3.00 g) and sodium iodide (6.43 g) in acetone (23.9 mL) was stirred at 60° C. for 5 hours. After insoluble materials were filtered off, the filtrate was concentrated in vacuo. Flash chromatography (silica, hexane:ethyl acetate=5:1) of the residue gave AD (3.10 g).

¹H NMR (CDCl₃): δ 1.42 (s, 9H), 1.62-2.17 (m, 10H), 3.14-3.18 (m, 2H), 3.90 (s, 2H), 4.25 (brs, 1H).

MS (ESI⁺) m/z: 382 (MH⁺).

HRMS (ESI⁺) for C₁₄H₂₅INO₃ (MH⁺): calcd, 382.08791. found, 382.08833.

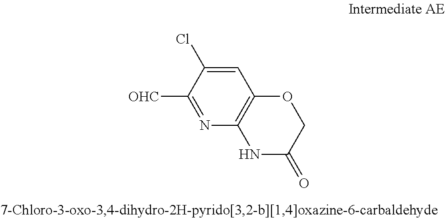 [SMILES: O=Cc1nc2c(cc1Cl)OCC(=O)N2]

This reagent was prepared according to the procedure described in International Patent Publication No. WO 2006020561.

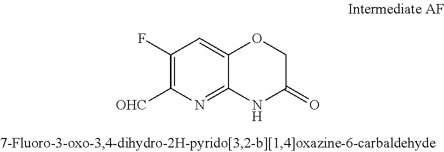 [SMILES: O=Cc1nc2c(cc1F)OCC(=O)N2]

Step 1

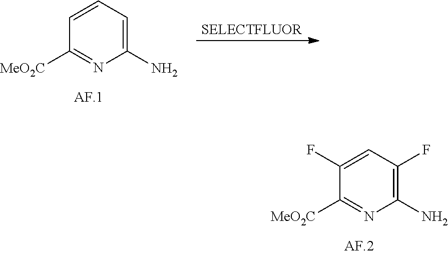 [SMILES: COC(=O)c1cccc(N)n1 ; "SELECTFLUOR" ; COC(=O)c1nc(N)c(F)cc1F]

To a solution of AF.1 (18.0 g) in acetonitrile (590 mL) was added SELECTFLUOR (41.9 g) at room temperature, the mixture was stirred at the same temperature for 4 days and then concentrated in vacuo. After dilution of the residue with ethyl acetate, the mixture was washed with saturated sodium hydrogencarbonate solution. The organic extracts were washed with brine, dried over anhydrous sodium sulfate, filtered, and then concentrated in vacuo. Flash chromatography (silica, hexane:ethyl acetate=1:1) of the residue gave AF.2 (1.10 g).

¹H NMR (CDCl₃): δ 3.97 (s, 3H), 4.79 (br, 2H), 7.19 (t, J=9.2 Hz, 1H).

MS (EI⁺) m/z: 188 (M⁺).

HRMS (EI⁺) for C₇H₆F₂N₂O₂ (M⁺): calcd, 188.0397. found, 188.0424.

Step 2

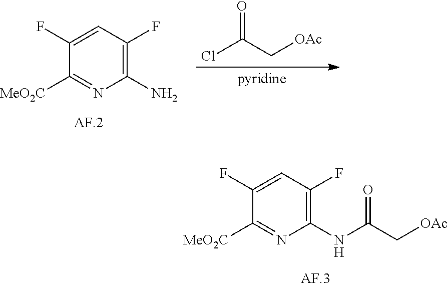

To a solution of AF.2 (590 mg) in pyridine (12.5 mL) was added acetoxyacetyl chloride (0.37 mL) under cooling with ice, the mixture was stirred at room temperature for 24 hours and then concentrated in vacuo. After dilution of the residue with ethyl acetate, the mixture was washed with saturated sodium hydrogencarbonate solution. The organic extracts were dried over anhydrous sodium sulfate, filtered, and then concentrated in vacuo. Flash chromatography (silica, hexane:ethyl acetate=1:1) of the residue gave methyl AF.3 (630 mg).

¹H NMR (CDCl₃): δ 2.22 (s, 3H), 4.01 (s, 3H), 4.87 (br, 2H), 7.45 (t, J=8.6 Hz, 1H), 8.17 (br, 1H).

MS (EI⁺) m/z: 288 (M⁺).

HRMS (EI⁺) for C₁₁H₁₀F₂N₂O₅ (M⁺): calcd, 288.0558. found, 288.0544.

Step 3

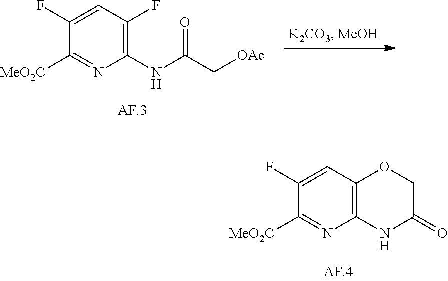

To a solution of AF.3 (625 mg) in methanol (43 mL) was added potassium carbonate (600 mg) under cooling with ice, then mixture was stirred at room temperature for 1 hour and then concentrated in vacuo. After dilution of the residue with ethyl acetate, the mixture was washed with water and 10% citric acid solution. The organic extracts were dried over anhydrous sodium sulfate, filtered, and then concentrated in vacuo. Flash chromatography (silica, hexane:ethyl acetate=3:2) of the residue gave AF.4 (150 mg).

¹H NMR (DMSO-d₆): δ 3.82 (s, 3H), 4.76 (s, 2H), 7.57 (d, J=11.0 Hz, 1H), 11.65 (s, 1H).

MS (EI⁺) m/z: 226 (M⁺).

HRMS (EI⁺) for C₉H₇FN₂O₄ (M⁺): calcd, 226.0390. found, 226.0377.

Step 4

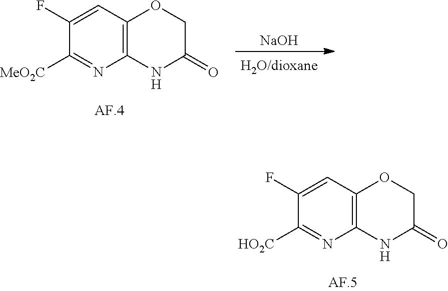 [SMILES: COC(=O)c1nc2c(cc1F)OCC(=O)N2 ; "NaOH" ; O=C1COc2cc(F)c(C(=O)O)nc2N1]

To a solution of AF.4 (370 mg) in 1,4-dioxane (55 mL) and water (14 mL) was added 0.5 N sodium hydroxide solution (3.7 mL) under cooling with ice, the mixture was stirred at room temperature for 12 hours and then concentrated in vacuo. After dilution of the residue with water, the mixture was washed with water and 10% citric acid solution. The organic extracts were dried over anhydrous sodium sulfate, filtered, and then concentrated in vacuo. After dilution of the residue with water, the resulting mixture was adjusted to pH 5 by addition of 1 N hydrochloric acid. The resulting precipitates were collected by filtration to give AF.5 (154 mg).

¹H NMR (DMSO-d₆): δ 4.62 (s, 2H), 7.24 (d, J=9.2 Hz, 1H), 11.17 (s, 1H).

MS (EI⁺) m/z: 212 (M⁺).

HRMS (EI⁺) for C₈H₅FN₂O₄ (M⁺): calcd, 212.0233. found, 212.0243.

Step 5

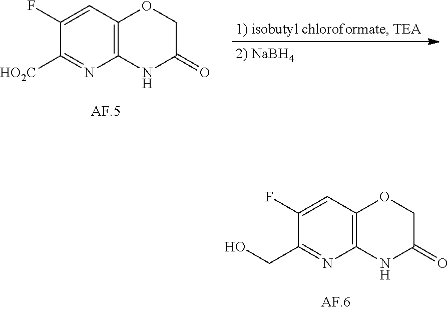 [SMILES: O=C1COc2cc(F)c(C(=O)O)nc2N1 ; O=C1COc2cc(F)c(CO)nc2N1]

To a solution of AF.5 (300 mg) and triethylamine (0.45 mL) in N,N-dimethylformamide (14 mL) was added isobutyl chloroformate (0.20 mL) at −10° C., the mixture was stirred at the same temperature for 30 minutes. The insoluble materials were filtered off. To a suspension of sodium borohydride (161 mg) in water (7 mL) was added the filtrate thus obtained under cooling with ice, the mixture was stirred at room temperature for 30 minutes. The resulting mixture was adjusted to pH 7 by addition of 1 N hydrochloric acid and then concentrated in vacuo. Flash chromatography (silica, hexane:ethyl acetate=1:4) of the residue gave AF.6 (82.2 mg).

¹H NMR (DMSO-d₆): δ 4.42 (dd, J=5.5, 2.4 Hz, 2H), 4.65 (s, 2H), 5.18 (t, J=5.5 Hz, 1H), 7.42 (d, J=9.7 Hz, 1H), 11.32 (s, 1H).

MS (EI⁺) m/z: 198 (M⁺).

HRMS (EI⁺) for C₈H₇FN₂O₃ (M⁺): calcd, 198.0441. found, 198.0475.

Step 6

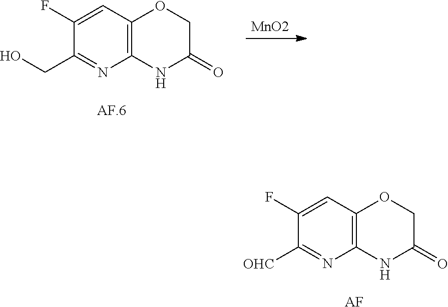

To a solution of AF.6 (80.0 mg) in tetrahydrofuran (6 mL) was added manganese dioxide (281 mg), the mixture was stirred at room temperature for 2 hours and further stirred at 60° C. for 3 hours. After insoluble materials were filtered off, the filtrate was concentrated in vacuo. Treatment of the residue with diethyl ether gave AF (64.5 mg).

¹H NMR (DMSO-d₆): δ 4.80 (s, 2H), 7.60 (d, J=11.0 Hz, 1H), 9.90 (s, 1H), 11.70 (s, 1H).

MS (EI⁺) m/z: 196 (M⁺).

HRMS (EI⁺) for C₈H₅FN₂O₃ (M⁺): calcd, 196.0284. found, 196.0293.

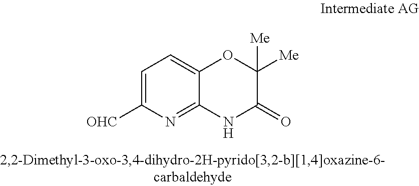 [SMILES: CC1(C)Oc2ccc(C=O)nc2NC1=O]

Step 1

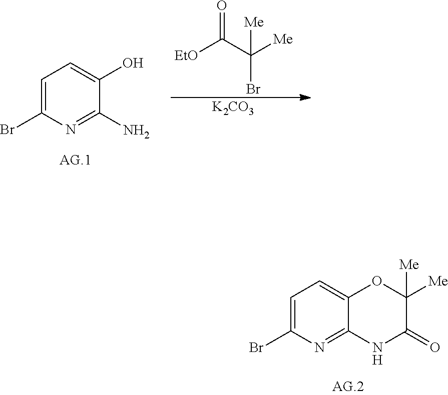

A suspension of AG.1 (1.0 g) and potassium carbonate (2.24 g) in acetone (21 mL) was added ethyl 2-bromo-2-methylpropanoate (1.1 mL), the mixture was stirred under reflux for 9 hours, and then concentrated in vacuo. After dilution of the residue with dichloromethane/methanol, the mixture was washed with water. The organic extracts were washed with brine, dried over anhydrous sodium sulfate, filtered, and then concentrated in vacuo. Treatment of the residue with ethanol gave AG.2 (976 mg).

¹H NMR (DMSO-d₆): δ 1.41 (s, 6H), 7.17 (d, J=7.9 Hz, 1H), 7.32 (d, J=8.6 Hz, 1H).

MS (EI⁺) m/z: 256 (M⁺).

HRMS (EI⁺) for C₉H₉BrN₂O₂ (M⁺): calcd, 255.9847. found, 255.9874.

Step 2

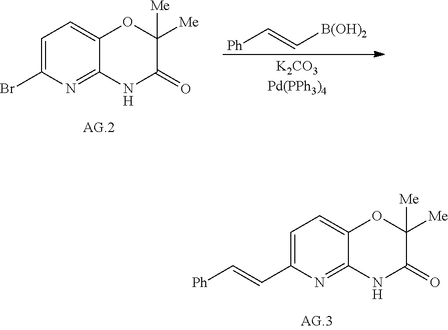 [SMILES: CC1(C)Oc2ccc(Br)nc2NC1=O ; OB(O)/C=C/c1ccccc1 ; CC1(C)Oc2ccc(/C=C/c3ccccc3)nc2NC1=O]

Compound AG.3 (780 mg) was prepared from AG.2 (900 mg) and (E)-styrylboronic acid (534 mg). To a degassed solution of AG.2 (900 mg) in 1,4-dioxane (17.5 mL) and water (14 mL) was added phenylvinylboronic acid (534 mg), potassium carbonate (967 mg) and tetrakis(triphenylphosphine)palladium (121 mg), the mixture was heated at reflux for 13.5 hours. After dilution of the mixture with water (30 mL), the resulting precipitates were collected by filtration and washed with water. Flash chromatography (silica, hexane:ethyl acetate=2:1) of the crude product gave AG.3 (780 mg).

¹H NMR (DMSO-d₆): δ 1.43 (s, 6H), 7.15 (d, J=7.9 Hz, 1H), 7.20 (d, J=16.3 Hz, 1H), 7.26-7.31 (m, 1H), 7.34 (d, J=7.9 Hz, 1H), 7.37 (d, J=7.9 Hz, 1H), 7.45 (d, J=16.3 Hz, 1H), 7.58 (d, J=7.3 Hz, 2H), 11.19 (br, 1H).

MS (EI⁺) m/z: 280 (M⁺).

HRMS (EI⁺) for C₁₇H₁₆N₂O₂ (M⁺): calcd, 280.1212. found, 280.1218.

Step 3

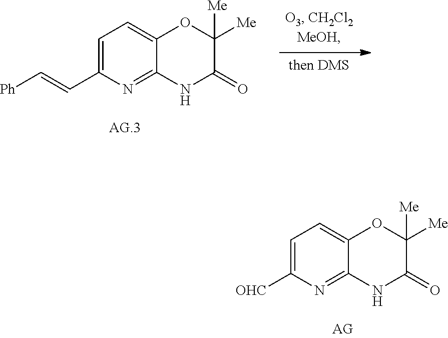 [SMILES: CC1(C)Oc2ccc(/C=C/c3ccccc3)nc2NC1=O ; CC1(C)Oc2ccc(C=O)nc2NC1=O]

A solution of A.3 (600 mg) in dichloromethane (25 mL) and methanol (9 mL) was cooled to −78° C. Ozone was bubbled through the solution with stirring for 40 minutes, and then the excess ozone was removed by bubbling air through the solution for 10 minutes. Dimethyl sulfide (0.79 mL) was added to the mixture. The resulting mixture was stirred at room temperature for 50 minutes and then concentrated in vacuo. Treatment of the residue with diethyl ether and 0.1 M hydrochloric acid gave AG (365 mg).

¹H NMR (DMSO-d₆): δ 1.47 (s, 6H), 7.53 (d, J=8.4 Hz, 1H), 7.62 (d, J=7.9 Hz, 1H), 9.79 (s, 1H), 11.60 (br, 1H).

MS (EI⁺) m/z: 206 (M⁺).

HRMS (EI⁺) for C₁₀H₁₀N₂O₃ (M⁺): calcd, 206.0691. found, 206.0666.

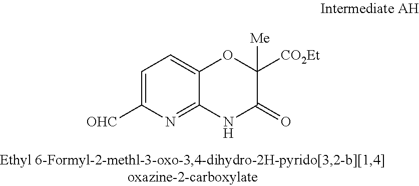 [SMILES: CCOC(=O)C1(C)Oc2ccc(C=O)nc2NC1=O]

Step 1

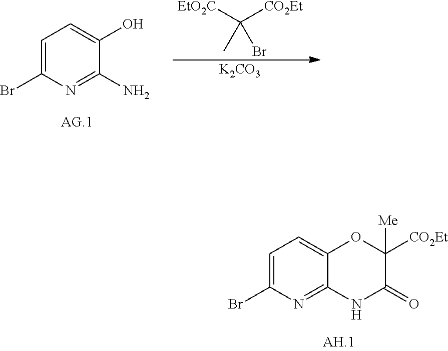 [SMILES: Nc1nc(Br)ccc1O ; "K2CO3" ; CCOC(=O)C(C)(Br)C(=O)OCC ; CCOC(=O)C1(C)Oc2ccc(Br)nc2NC1=O]

To a mixture of diethyl 2-bromo-2-methylmalonate (1.07 g) and potassium fluoride (0.58 g) in N,N-dimethylformamide (3 mL) was added a solution of AG.1 (0.24 g) in N,N-dimethylformamide (1 mL), the mixture was stirred at 60° C. for 3 hours, and then concentrated in vacuo. Flash chromatography (silica, toluene:ethyl acetate=3:1) of the residue gave AH.1 (0.32 g).

¹H NMR (DMSO-d₆): δ 1.07 (t, J=7.9 Hz, 3H), 1.71 (s, 3H), 4.08-4.15 (m, 2H), 7.22 (d, J=9.2 Hz, 1H), 7.43 (d, J=9.2 Hz, 1H), 11.82 (s, 1H).

MS (EI⁺) m/z: 314 (M⁺).

Step 2

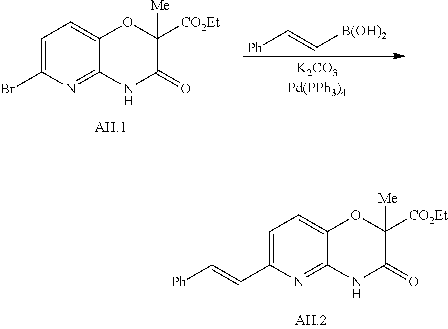 [SMILES: CCOC(=O)C1(C)Oc2ccc(Br)nc2NC1=O ; OB(O)/C=C/c1ccccc1 ; CCOC(=O)C1(C)Oc2ccc(/C=C/c3ccccc3)nc2NC1=O]

To a degassed solution of AH.1 (500 mg) in N,N-dimethylformamide (16 mL) was added phenylvinylboronic acid (484 mg), potassium carbonate (448 mg) and tetrakis(triphenylphosphine)palladium (55.7 mg), the mixture was heated at 100° C. for 15 hours, and then concentrated in vacuo. After dilution of the residue with ethyl acetate, the mixture was washed with water. The organic extracts were washed with brine, dried over anhydrous sodium sulfate, filtered, and then concentrated in vacuo. Flash chromatography (silica, hexane:ethyl acetate=3:1) of the crude product gave AH.2 (439 mg).

¹H NMR (DMSO-d₆): δ 1.08 (t, J=7.3 Hz, 3H), 1.71 (s, 3H), 4.05-4.17 (m, 2H), 7.18 (d, J=8.6 Hz, 1H), 7.20 (d, J=15.9 Hz, 1H), 7.24-7.31 (m, 1H), 7.36-7.48 (m, 4H), 7.58 (d, J=7.3 Hz, 2H), 11.56 (br, 1H).

MS (EI⁺) m/z: 338 (M⁺).

HRMS (EI⁺) for C₁₉H₁₈N₂O (M⁺): calcd, 338.1267. found, 338.1281.

Step 3

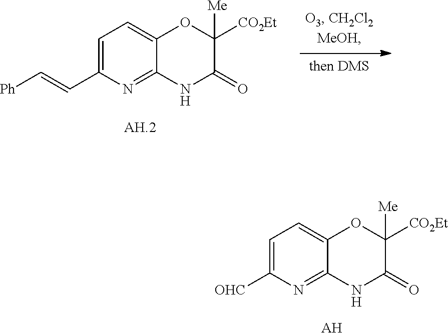 [SMILES: CCOC(=O)C1(C)Oc2ccc(/C=C/c3ccccc3)nc2NC1=O ; CCOC(=O)C1(C)Oc2ccc(C=O)nc2NC1=O ; "AH"]

A solution of AH.2 (430 mg) in dichloromethane (15 mL) and methanol (5 mL) was cooled to −60° C. Ozone was bubbled through the solution with stirring for 40 minutes, and then the excess ozone was removed by bubbling air through the solution for 10 minutes. Dimethyl sulfide (0.47 mL) was added to the mixture. The resulting mixture was stirred at room temperature for 50 minutes and then concentrated in vacuo. Treatment of the residue with diethyl ether gave AH (207 mg).

¹H NMR (DMSO-d₆): δ 1.07 (t, J=7.3 Hz, 3H), 1.76 (s, 3H), 4.08-4.16 (m, 2H), 7.63 (d, J=7.9 Hz, 1H), 7.66 (d, J=7.9 Hz, 1H), 9.79 (s, 1H), 11.98 (br, 1H).

MS (EI⁺) m/z: 264 (M⁺).

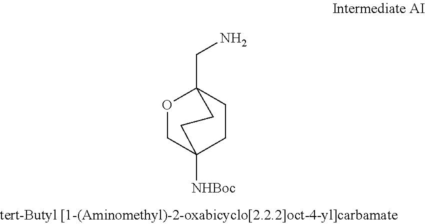 [SMILES: CC(C)(C)OC(=O)NC12CCC(CN)(CC1)OC2]

Step 1

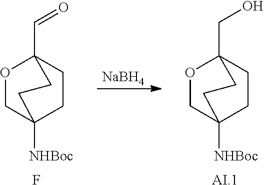

To a solution of F (255 mg) in methanol (5 mL) was added sodium borohydride (76 mg) at 0° C. and the mixture was stirred at room temperature. Concentrated, the residue was dissolved with ethyl acetate and washed with water and brine, dried over anhydrous sodium sulfate and condensed to give crude AI.1 and used directly.

Step 2

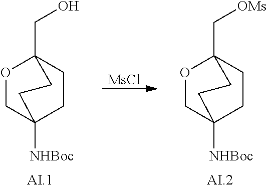

A solution of AI.1 (220 mg) and triethylamine (130.5 mg) in anhydrous dichloromethane (5 mL) was added methanesulfonyl chloride (118 mg). The mixture was stirred for 1 hour and then washed subsequently with saturated aqueous sodium hydrogencarbonate, water and brine, dried over anhydrous sodium sulfate, and condensed to give AI.2 (200 mg).

¹H NMR (CDCl₃): δ 1.35 (s, 9H), 1.59-1.66 (m, 2H), 1.75-1.81 (m, 2H), 1.90-1.96 (m, 2H), 2.03-2.08 (m, 2H), 2.99 (s, 3H), 3.90 (s, 2H), 3.97 (s, 2H), 4.25 (s, 1H).

Step 3

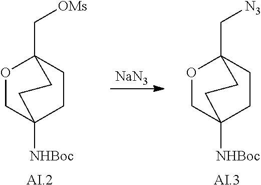

A mixture of AI.2 (200 mg), sodium azide (43 mg) and sodium iodide (15 mg) in dimethyl sulfoxide (4 mL) was stirred overnight at 100° C. After dilution of the residue with ethyl acetate, the mixture was washed with water thrice and brine, dried over anhydrous sodium sulfate and condensed. The residue was purified by prep-TLC (petroleum ether:ethyl acetate=10:1) to afford pure AI.3.

¹H NMR (CDCl₃): δ 1.35 (s, 9H), 1.50-1.61 (m, 2H), 1.64-1.81 (m, 2H), 1.87-1.94 (m, 2H), 1.98-2.15 (m, 2H), 3.06 (s, 2H), 3.89 (s, 2H), 4.34 (s, 1H).

Step 4

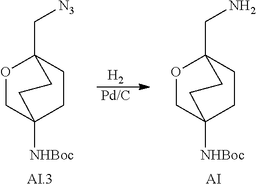

A mixture of compound AI.3 (180 mg) and 10% Pd/C (20 mg) in ethyl acetate (5 mL) and acetic acid (0.5 mL) was stirred under 15 psi of hydrogen at room temperature for 5 hours. Filtrated and condensed to afford pure AI (126 mg).

¹H NMR (CDCl₃): δ 1.35 (s, 9H), 1.54-1.61 (m, 2H), 1.76-1.81 (m, 2H), 1.87-1.94 (m, 2H), 2.00-2.06 (m, 2H), 3.06 (s, 2H), 3.88 (s, 2H), 4.29 (s, 1H).

MS m/z: 257 (MH⁺).

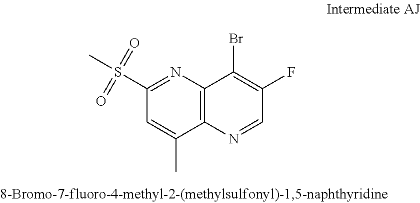 [SMILES: Cc1cc(S(C)(=O)=O)nc2c(Br)c(F)cnc12]

Step 1

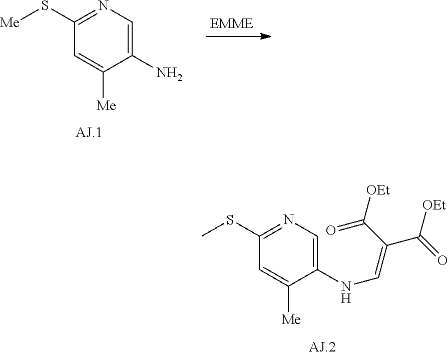 [SMILES: CSc1cc(C)c(N)cn1 ; CCOC(=O)C(=CNc1cnc(SC)cc1C)C(=O)OCC]

A mixture of AJ.1 (3.1 g) and diethyl ethoxymethylenemalonate (4.3 g) in toluene (80 mL) was refluxed for 1 hour. Concentrated to dryness afforded a solid which was used directly for the next step. MS m/z: 325 (MH⁺).

Step 2

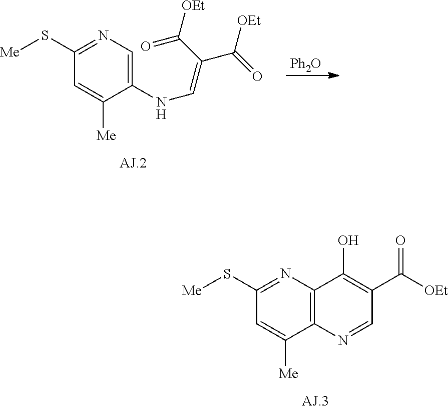

Compound AJ.2 (5.7 g, crude) was added portionwise to diphenyl ether (30 mL) at 260° C. and refluxed for 8 minutes. The mixture was cooled to 60° C. and diluted with petroleum ether. The resulting precipitates were collected by filtration and washed with petroleum ether to give crude AJ.3 (2.8 g). MS m/z: 279 (MH⁺).

Step 3

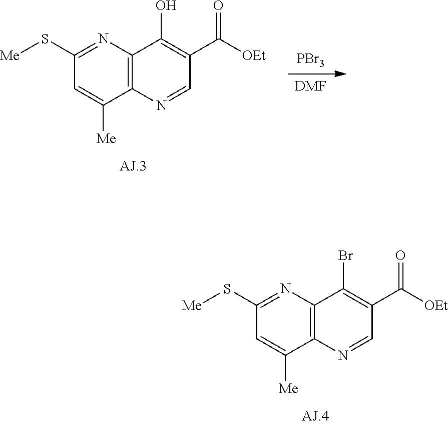

To a suspension of AJ.3 (2.8 g, crude) in N,N-dimethylformamide (40 mL) was added phosphorous tribromide (3.2 g) under cooling with water. The mixture was stirred at room temperature for 30 minutes then poured into ice water, and adjust to pH 10 by addition of saturated sodium hydrogencarbonate solution. The resulting precipitates were collected by filtration and washed with water. The wet cake (2.5 g) was used directly for the next step. MS m/z: 341 (MH⁺).

Step 4

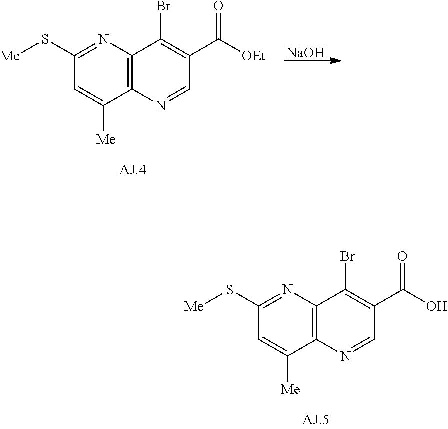

To the solution of AJ.4 (2.5 g wet in 30 mL of tetrahydrofuran) was added a solution of sodium hydroxide (0.5 g in 10 mL of water) slowly. The mixture was stirred overnight at room temperature. Concentrated and acidified to pH 5 with concentrated hydrochloric acid. The white precipitate was collected by filtration, washed with water and dried under vacuum to afford pure AJ.5 (1.8 g). MS m/z: 315 (MH⁺).

Step 5

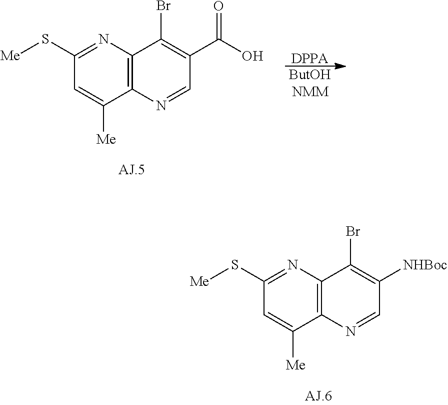

A mixture of AJ.5 (1.6 g) and N-methylmorpholine (0.6 g) in 1,2-dichloroethane (60 mL) was stirred at room temperature for 15 minutes. Diphenyl phosphoryl azide (1.7 g) was added dropwise to the clear solution and stirred for 30 minutes then refluxed for another 75 minutes. To the reaction mixture was added tert-butanol (20 mL) and refluxed overnight before cooled down. The reaction mixture was diluted with dichloromethane (300 mL), washed with water and brine, and concentrated. The residue was purified by column chromatography (20% ethyl acetate in petroleum ether) to give AJ.6 (1.3 g). MS m/z: 386 (MH⁺).

Step 6

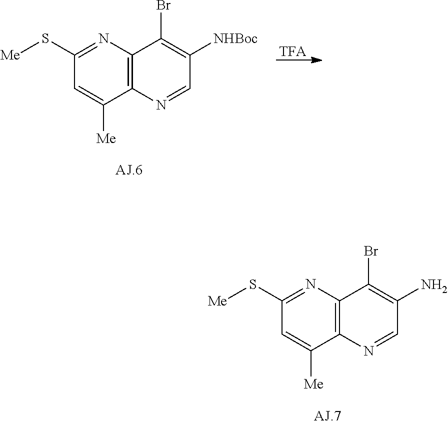 [SMILES: CSc1cc(C)c2ncc(NC(=O)OC(C)(C)C)c(Br)c2n1 ; CSc1cc(C)c2ncc(N)c(Br)c2n1 ; "TFA"]

To a solution of AJ.6 (1.3 g) in dichloromethane (15 mL) was added trifluoroacetic acid (15 mL) and the mixture was stirred overnight at room temperature. Concentrated, residue was dissolved in ethyl acetate (200 mL) and washed subsequently with saturated sodium carbonate, water and brine. The ethyl acetate layer was dried over anhydrous sodium sulfate and concentrated to give pure AJ.7 (0.9 g). MS m/z: 286 (MH⁺).

Step 7

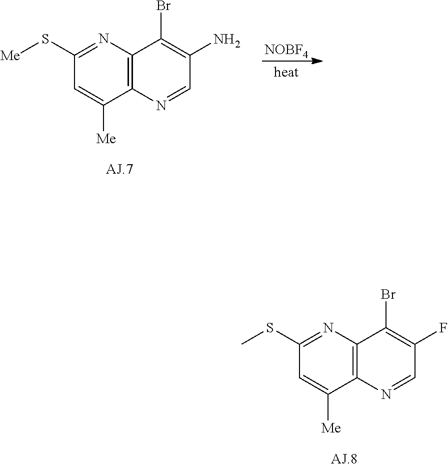

To an ice-cooled solution of AJ.7 (230 mg) in dry tetrahydrofuran (10 mL) was added nitrosyl tetrafluoroborate (140 mg). The mixture was stirred at 0° C. for 50 minutes then filtrated. The solid cake was washed with cold tetrahydrofuran (1 mL) and dried by vacuum at room temperature to afford a brown powder. This powder was suspended in decaline was heated to 100° C. for 1 hour. Cooled down, diluted with petroleum ether (100 mL) and filtrated through a silica gel pad washed with petroleum ether to remove the decaline then washed with dichloromethane to afford a white solid (140 mg). MS m/z: 287 (MH⁺).

Step 8

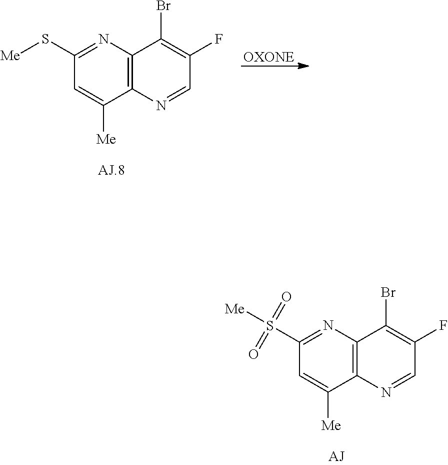 [SMILES: CSc1cc(C)c2ncc(F)c(Br)c2n1 ; "OXONE" ; Cc1cc(S(C)(=O)=O)nc2c(Br)c(F)cnc12]

A suspension of AJ.8 (140 mg) and OXONE (1 g) in methanol/tetrahydrofuran/water (5 mL5 mL5 mL) was stirred at room temperature for 3 hours. Concentrated, the residue was washed with water and dried under vacuum to afford AJ as a white solid (130 mg). MS m/z: 319 (MH⁺).

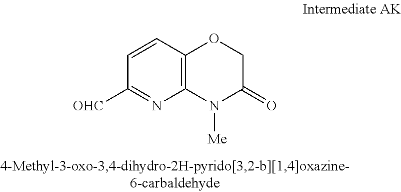 [SMILES: CN1C(=O)COc2ccc(C=O)nc21]

To a suspension of I (35.6 mg), potassium carbonate (69.1 mg) and benzyl triethylammonium chloride (45.6 mg) in acetonitrile (1 mL), iodomethane (12.5 μL) was added and the mixture stirred at room temperature for 2 hours. After insoluble materials were filtered off, the filtrate was concentrated in vacuo. Dichloromethane solution of the residue was washed with 10% citric acid solution, saturated hydrogencarbonate solution and brine. The organic extracts were dried over anhydrous sodium sulfate, filtered, and then concentrated in vacuo. Flash chromatography (silica, toluene:ethyl acetate=2:1) of the residue gave 4-methyl-3-oxo-3,4-dihydro-2H-pyrido[3,2-b][1,4]oxazine-6-carbaldehyde (34.1 mg).

¹H NMR (DMSO-d₆): δ 3.39 (s, 3H), 4.90 (s, 2H), 7.53 (d, J=8.6 Hz, 1H), 7.65 (d, J=8.6 Hz, 1H), 9.85 (s, 1H).

MS (EI⁺) m/z: 192 (M⁺).

HRMS (EI⁺) for C₉H₈N₂O₃ (M): calcd, 192.0535. found, 192.0537.

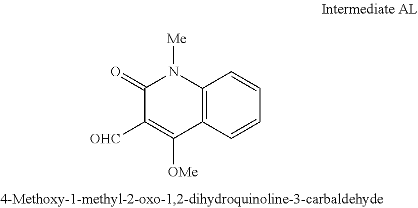 [SMILES: COc1c(C=O)c(=O)n(C)c2ccccc12]

The title compound was prepared according to methods described in Kobayashi et al., 2009, Tetrahedron Lett. 50:6665-6667.

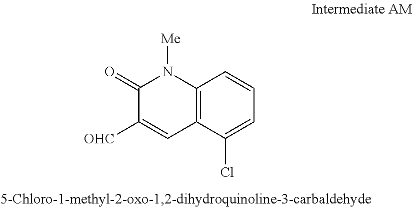 [SMILES: Cn1c(=O)c(C=O)cc2c(Cl)cccc21]

Step 1

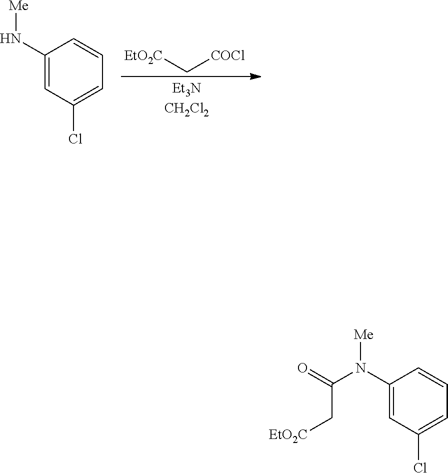 [SMILES: CNc1cccc(Cl)c1 ; CCOC(=O)CC(=O)Cl ; CCOC(=O)CC(=O)N(C)c1cccc(Cl)c1]

To a solution of 3-chloro-N-methylaniline (3.61 g) and triethylamine (5.34 mL) in dichloromethane (100 mL) was added ethyl 3-chloro-3-oxopropanoate (5.00 g) under cooling with ice, the mixture was stirred at room temperature for 2 hours. The mixture was washed with 1 N hydrochloric acid and water. The organic extracts were dried over anhydrous sodium sulfate, filtered, and then concentrated in vacuo. Flash chromatography (silica, hexane:ethyl acetate=2:1) of the residue gave ethyl 3-((3-chlorophenyl)(methyl)amino)-3-oxopropanoate (6.52 g).

¹H NMR (CDCl₃): δ 1.25 (t, J=6.7 Hz, 3H), 3.30 (s, 3H), 4.14 (q, J=7.3 Hz, 2H), 7.16 (m, 1H), 7.27 (m, 1H), 7.38 (m, 1H).

MS (EI⁺) m/z: 255 (M⁺).

HRMS (EI⁺) for C₁₂H₁₄ClNO₃ (M⁺): calcd, 255.0662. found, 255.0659.

Step 2

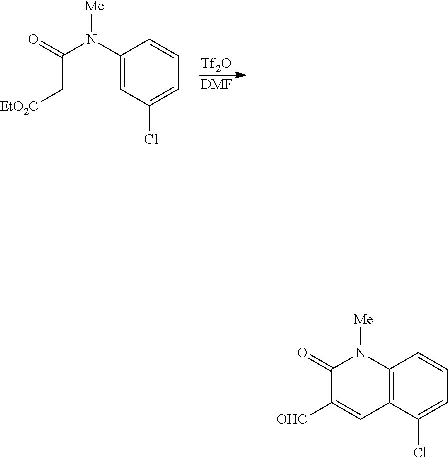

To a solution of ethyl 3-((3-chlorophenyl)(methyl)amino)-3-oxopropanoate (2.56 g) in N,N-dimethylformamide (10 mL) was added triflic anhydride (5.1 mL) at −10° C., the mixture stirred at room temperature for 15 hours. The mixture was poured into ice water. The resulting precipitates were collected by filtration and washed with ethanol. The filtrate was concentrated in vacuo. Flash chromatography (silica, hexane:ethyl acetate=1:1) of the residue gave 5-chloro-1-methyl-2-oxo-1,2-dihydroquinoline-3-carbaldehyde (15.6 mg).

¹H NMR (CDCl₃): δ 3.77 (s, 3H), 7.32 (d, J=8.6 Hz, 1H), 7.36 (d, J=7.9 Hz, 1H), 7.60 (t, J=8.6 Hz, 1H), 8.82 (s, 1H), 10.49 (s, 1H).

MS (EI⁺) m/z: 221 (M⁺).

HRMS (EI⁺) for C₁₁H₈ClNO₂ (M⁺): calcd, 221.0244. found, 221.0265.

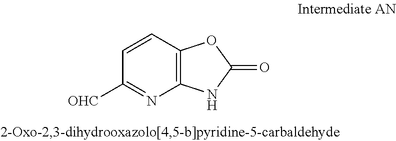 [SMILES: O=Cc1ccc2oc(=O)[nH]c2n1]

Step 1

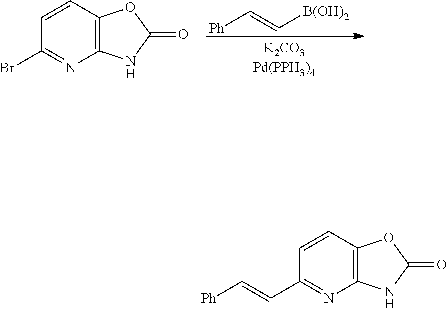

To a degassed solution of 5-bromooxazolo[4,5-b]pyridin-2(3H)-one (430 mg, prepared according to the literature; International Patent Application Publication No. WO 2008148449) in 1,4-dioxane (10 mL) and water (8 mL) was added phenylvinylboronic acid (305 mg), potassium carbonate (553 mg) and tetrakis(triphenylphosphine)palladium (69.3 mg); the mixture was heated at reflux for 17 hours, and then concentrated in vacuo. After dilution of the residue with dichloromethane, the mixture was washed with aqueous ammonium chloride solution. The organic extracts were washed with brine, dried over anhydrous sodium sulfate, filtered, and then concentrated in vacuo. Flash chromatography (silica, hexane: 1,4-dioxane=2:1) of the residue gave styryloxazolo[4,5-b]pyridin-2(3H)-one (265 mg).

¹H NMR (DMSO-d₆): δ 7.22 (d, J=7.9 Hz, 1H), 7.26-7.33 (m, 2H), 7.36-7.42 (m, 2H), 7.48 (d, J=15.8 Hz, 1H), 7.62 (d, J=7.9 Hz, 3H), 12.42 (brs, 1H).

MS (EI⁺) m/z: 238 (M⁺).

HRMS (EI⁺) for C₁₄H₁₀N₂O₂ (M⁺): calcd, 238.0742. found, 238.0759.

Step 2

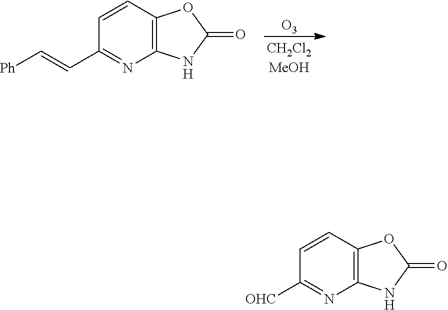

A suspension of styryloxazolo[4,5-b]pyridin-2(3H)-one (250 mg) in dichloromethane (12.4 mL) and methanol (4.5 mL) was bubbled with ozone at −65° C. until a pale blue colour appeared. The excess ozone was removed by bubbling air through the suspension for 20 minutes. Dimethyl sulfide (0.39 mL) was added to the suspension. The mixture was stirred at room temperature for 1 hour and concentrated in vacuo. After dilution of the mixture with diethyl ether (2 mL) and 0.5 M hydrochloric acid (1 mL), the resulting precipitates were collected by filtration. Treatment of the crude product with diethyl ether gave 2-oxo-2,3-dihydrooxazolo[4,5-b]pyridine-5-carbaldehyde (148 mg).

¹H NMR (DMSO-d₆): δ 7.76 (d, J=8.6 Hz, 1H), 7.83 (d, J=7.9 Hz, 1H), 9.87 (s, 1H), 12.82 (brs, 1H).

MS (EI⁺) m/z: 164 (M⁺).

HRMS (EI⁺) for C₇H₄N₂O₃ (M⁺): calcd, 164.0222. found, 164.0217.

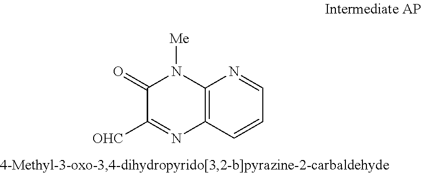 [SMILES: Cn1c(=O)c(C=O)nc2cccnc21]

Step 1

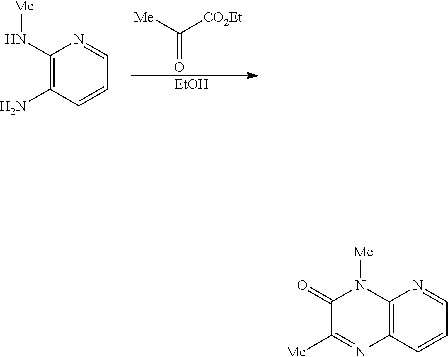 [SMILES: "EtOH" ; CNc1ncccc1N ; CCOC(=O)C(C)=O ; Cc1nc2cccnc2n(C)c1=O]

A mixture of N²-methylpyridine-2,3-diamine (0.30 g) and ethyl pyruvate (0.31 g) in ethanol (4 mL) was heated under reflux for 5 hours. After dilution of the mixture with water, the mixture was extracted with ethyl acetate. The organic extracts were dried over anhydrous sodium sulfate, filtered, and then concentrated in vacuo. Flash chromatography (silica, hexane:ethyl acetate=3:1) of the residue gave 2,4-dimethylpyrido[3,2-b]pyrazin-3(4H)-one (0.32 g).

¹H NMR (DMSO-d₆): δ 2.49 (s, 3H), 3.69 (s, 3H), 7.44 (dd, J=7.9, 4.8 Hz, 1H), 8.20 (dd, J=7.9, 1.2 Hz, 1H), 8.62 (dd, J=4.2, 1.2 Hz, 1H).

MS (EI⁺) m/z: 175 (M⁺).

Step 2

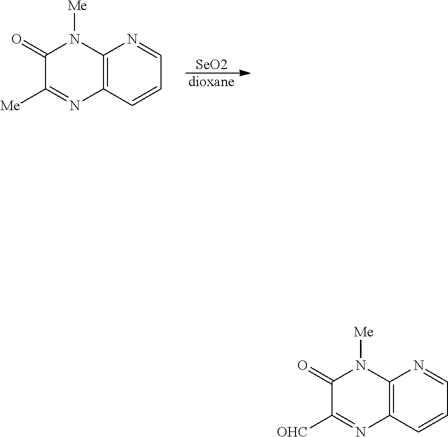

A suspension of 2,4-dimethylpyrido[3,2-b]pyrazin-3(4H)-one (10.0 g) and selenium dioxide (13.3 g) in 1,4-dioxane (500 mL) was heated under reflux for 3 hours. The resulting insoluble materials were filtered off. After dilution of the filtrate with water, the mixture was extracted with ethyl acetate. The organic extracts were washed with water and brine, dried over anhydrous sodium sulfate, filtered, and then concentrated in vacuo to give 4-methyl-3-oxo-3,4-dihydropyrido[3,2-b]pyrazine-2-carbaldehyde (9.00 g).

¹H NMR (DMSO-d₆): δ 3.68 (s, 3H), 7.54 (dd, J=7.9, 4.3 Hz, 1H), 8.43 (dd, J=7.9, 1.2 Hz, 1H), 8.78 (dd, J=4.3, 1.2 Hz, 1H), 10.24 (s, 1H).

MS (CI⁺) m/z: 190 (MH⁺).

### EXAMPLES

Many of the following compounds were prepared in a pharmaceutically acceptable salt form (e.g. amine hydrochloride) for use in characterization, ease of handling, and use in subsequent transformations. It is within the purview of those skilled in the art to prepare the corresponding free base forms as well as alternative salts using well-known methods.

### Example 1

### 6-((4-(2-(6-Methoxy-1,5-naphthyridin-4-yl)ethyl)bicyclo[2.2.2]octan-1-ylamino)methyl)-2H-pyrido[3,2-b][1,4]oxazin-3(4H)-one Hydrochloride

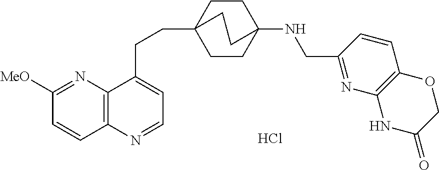 [SMILES: COc1ccc2nccc(CCC34CCC(NCc5ccc6c(n5)NC(=O)CO6)(CC3)CC4)c2n1 ; Cl]

### Step 1

### tert-Butyl 4-(2-(6-Methoxy-1,5-naphthyridin-4-yl)ethyl)bicyclo[2.2.2]octan-1-ylcarbamate

A degassed mixture of C (463 mg), 8-bromo-2-methoxy-1,5-naphthyridine (310 mg), cesium carbonate (1.27 g) and Pd PEPPSI-iPr (Sigma-Aldrich, St. Louis, Mo.) (35.2 mg) in tetrahydrofuran/water (9:1, 2.6 mL) was stirred at 100° C. in a sealed tube for 33 hours. After dilution of the reaction mixture with water, the mixture was extracted with dichloromethane. The organic extracts were washed with brine, dried over anhydrous sodium sulfate, filtered, and then concentrated in vacuo. Flash chromatography (silica, hexane:ethyl acetate=5:2) of the residue gave tert-butyl 4-(2-(6-methoxy-1,5-naphthyridin-4-yl)ethyl)bicyclo[2.2.2]octan-1-ylcarbamate (255 mg).

¹H NMR (CDCl₃): δ 1.43 (s, 9H), 1.50-1.54 (m, 2H), 1.58-1.67 (m, 6H), 1.79-1.95 (m, 6H), 3.03-3.07 (m, 2H), 4.06 (s, 3H), 4.34 (s, 1H), 7.10 (d, J=9.2 Hz, 1H), 7.33 (d, J=4.9 Hz, 1H), 8.17 (d, J=9.2 Hz, 1H), 8.63 (d, J=4.3 Hz, 1H).

MS (ESI⁺) m/z: 412 (MH⁺).

HRMS (ESI⁺) for C₂₄H₃₄N₃O₃ (MH⁺): calcd, 412.26002. found, 412.25963.

### Step 2

### 4-(2-(6-Methoxy-1,5-naphthyridin-4-yl)ethyl)bicyclo[2.2.2]octan-1-amine

To a solution of tert-butyl 4-(2-(6-methoxy-1,5-naphthyridin-4-yl)ethyl)bicyclo[2.2.2]octan-1-ylcarbamate (308 mg) in dichloromethane (3.1 mL) was added trifluoroacetic acid (3.1 mL) at 0° C., the mixture was stirred at the same temperature for 1.5 hours and then concentrated in vacuo. After dilution of the residue with water, the mixture was adjusted to pH 11 by adding 1 N sodium hydroxide solution. The aqueous mixture was extracted with dichloromethane/methanol (10:1). The organic extracts were washed with 1 N sodium hydroxide solution and brine, dried over anhydrous sodium sulfate, filtered, and then concentrated in vacuo to give 4-(2-(6-methoxy-1,5-naphthyridin-4-yl)ethyl)bicyclo[2.2.2]octan-1-amine (177 mg).

¹H NMR (DMSO-d₆): δ 1.16 (s, 2H), 1.35-1.58 (m, 14H), 2.95-3.04 (m, 2H), 4.00 (s, 3H), 7.22 (d, J=9.2 Hz, 1H), 7.49 (d, J=4.9 Hz, 1H), 8.21 (d, J=8.6 Hz, 1H), 8.62 (d, J=4.3 Hz, 1H).

MS (ESI⁺) m/z: 312 (MH⁺).

HRMS (ESI⁺) for C₁₉H₂₆N₃O (MH⁺): calcd, 312.20759. found, 312.20769.

### Step 3

### 6-((4-(2-(6-Methoxy-1,5-naphthyridin-4-yl)ethyl)bicyclo[2.2.2]octan-1-ylamino)methyl)-2H-pyrido[3,2-b][1,4]oxazin-3(4H)-one

A mixture of 4-(2-(6-methoxy-1,5-naphthyridin-4-yl)ethyl)bicyclo[2.2.2]octan-1-amine (165 mg), I (104 mg) and 3 Å molecular sieves (99 mg) in chloroform (2.1 mL) and methanol (2.1 mL) was heated under reflux for 1 hour. To the resulting mixture was added sodium triacetoxyborohydride (426 mg) at 0° C., the mixture was stirred at room temperature for overnight. After insoluble materials were filtered off, the filtrate was washed with sodium carbonate solution, dried over anhydrous sodium sulfate, filtered, and then concentrated in vacuo. Flash chromatography (silica, dichloromethane:methanol=8:1) of the residue gave 6-((4-(2-(6-methoxy-1,5-naphthyridin-4-yl)ethyl)bicyclo[2.2.2]octan-1-ylamino)methyl)-2H-pyrido[3,2-b][1,4]oxazin-3(4H)-one (200 mg).

¹H NMR (DMSO-d₆): δ 1.40-1.49 (m, 2H), 1.49-1.69 (m, 13H), 2.95-3.06 (m, 2H), 3.60 (s, 2H), 4.01 (s, 3H), 4.58 (s, 2H), 7.00 (d, J=8.6 Hz, 1H), 7.23 (d, J=9.2 Hz, 1H), 7.26 (d, J=8.6 Hz, 1H), 7.50 (d, J=4.3 Hz, 1H), 8.21 (d, J=9.2 Hz, 1H), 8.63 (d, J=4.3 Hz, 1H), 11.14 (s, 1H).

MS (ESI⁺) m/z: 474 (MH⁺).

HRMS (ESI⁺) for C₂₇H₃₂N₅O₃ (MH⁺): calcd, 474.25051. found, 474.25119.

### Step 4

### 6-((4-(2-(6-Methoxy-1,5-naphthyridin-4-yl)ethyl)bicyclo[2.2.2]octan-1-ylamino)methyl)-2H-pyrido[3,2-b][1,4]oxazin-3(4H)-one Hydrochloride

The title compound 6-((4-(2-(6-methoxy-1,5-naphthyridin-4-yl)ethyl)bicyclo[2.2.2]octan-1-ylamino)methyl)-2H-pyrido[3,2-b][1,4]oxazin-3(4H)-one hydrochloride (172 mg) was prepared from 6-((4-(2-(6-methoxy-1,5-naphthyridin-4-yl)ethyl)bicyclo[2.2.2]octan-1-ylamino)methyl)-2H-pyrido[3,2-b][1,4]oxazin-3(4H)-one (180 mg) in the same manner as described for Step 4 of EXAMPLE 3.

¹H NMR (DMSO-d₆): δ 1.45-1.55 (m, 2H), 1.57-1.77 (m, 6H), 1.85-2.00 (m, 6H), 2.93-3.09 (m, 2H), 4.02 (s, 3H), 4.04-4.07 (m, 2H), 4.68 (s, 2H), 7.23 (d, J=7.9 Hz, 1H), 7.24 (d, J=9.2 Hz, 1H), 7.45 (d, J=7.9 Hz, 1H), 7.53 (d, J=4.3 Hz, 1H), 8.23 (d, J=9.2 Hz, 1H), 8.65 (d, J=4.3 Hz, 1H), 8.96 (brs, 2H), 11.31 (s, 1H).

MS (ESI⁺) m/z: 474 (MH⁺) (as free base).

HRMS (ESI⁺) for C₂₇H₃₂N₅O₃ (MH⁺) (as free base): calcd, 474.25051. found, 474.25081.

### Example 2

### Ethyl 6-Methoxy-4-(2-(4-((3-oxo-3,4-dihydro-2H-pyrido[3,2-b][1,4]oxazin-6-yl)methylamino)bicyclo[2.2.2]octan-1-yl)ethyl)-1,5-naphthyridine-3-carboxylate

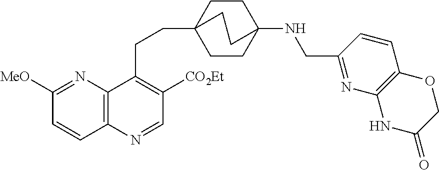 [SMILES: CCOC(=O)c1cnc2ccc(OC)nc2c1CCC12CCC(NCc3ccc4c(n3)NC(=O)CO4)(CC1)CC2]

### Step 1

### Ethyl 4-(2-(4-(tert-Butoxycarbonylamino)bicyclo[2.2.2]octan-1-yl)ethyl)-6-methoxy-1,5-naphthyridine-3-carboxylate

The title compound ethyl 4-(2-(4-(tert-butoxycarbonylamino)bicyclo[2.2.2]octan-1-yl)ethyl)-6-methoxy-1,5-naphthyridine-3-carboxylate (121 mg) was prepared from J (173 mg) and C (300 mg) in the same manner as described for Step 1 of EXAMPLE 1.

¹H NMR (CDCl₃): δ 1.45 (m, 14H), 1.62-1.73 (m, 6H), 1.78-1.95 (m, 6H), 3.43-3.53 (m, 2H), 4.08 (s, 3H), 4.34 (s, 1H), 4.45 (q, J=7.1 Hz, 2H), 7.16 (d, J=9.2 Hz, 1H), 8.18 (d, J=9.2 Hz, 1H), 9.10 (s, 1H).

MS (EI⁺) m/z: 483 (M⁺).

HRMS (EI⁺) for C₂₇H₃₇N₃O₅ (M⁺): calcd, 483.2733. found, 483.2692.

### Step 2

### Ethyl 4-(2-(4-Aminobicyclo[2.2.2]octan-1-yl)ethyl)-6-methoxy-1,5-naphthyridine-3-carboxylate

The title compound ethyl 4-(2-(4-aminobicyclo[2.2.2]octan-1-yl)ethyl)-6-methoxy-1,5-naphthyridine-3-carboxylate (75.4 mg) was prepared from ethyl 4-(2-(4-(tert-butoxycarbonylamino)bicyclo[2.2.2]octan-1-yl)ethyl)-6-methoxy-1,5-naphthyridine-3-carboxylate (102 mg) in the same manner as described for Step 2 of EXAMPLE 1.

¹H NMR (CDCl₃): δ 1.26-1.75 (m, 19H), 3.44-3.54 (m, 2H), 4.09 (s, 3H), 4.46 (q, J=7.1 Hz, 2H), 7.16 (d, J=9.2 Hz, 1H), 8.18 (d, J=9.2 Hz, 1H), 9.10 (s, 1H).

MS (ESI⁺) m/z: 384 (MH⁺).

HRMS (ESI⁺) for C₂₂H₃₀N₃O₃ (MH⁺): calcd, 384.22872. found, 384.22910.

### Step 3

### Ethyl 6-Methoxy-4-(2-(4-((3-oxo-3,4-dihydro-2H-pyrido[3,2-b][1,4]oxazin-6-yl)methylamino)bicyclo[2.2.2]octan-1-yl)ethyl)-1,5-naphthyridine-3-carboxylate

The title compound ethyl 6-methoxy-4-(2-(4-((3-oxo-3,4-dihydro-2H-pyrido[3,2-b][1,4]oxazin-6-yl)methylamino)bicyclo[2.2.2]octan-1-yl)ethyl)-1,5-naphthyridine-3-carboxylate (64.0 mg) was prepared from ethyl 4-(2-(4-aminobicyclo[2.2.2]octan-1-yl)ethyl)-6-methoxy-1,5-naphthyridine-3-carboxylate (71.4 mg) and I (34.8 mg) in the same manner as described for Step 3 of EXAMPLE 1.

¹H NMR (DMSO-d₆): δ 1.32-1.61 (m, 18H), 3.38 (m, 2H), 3.61 (s, 2H), 4.04 (s, 3H), 4.39 (q, J=7.1 Hz, 2H), 4.58 (s, 2H), 7.01 (d, J=7.9 Hz, 1H), 7.26 (d, J=7.9 Hz, 1H), 7.35 (d, J=9.2 Hz, 1H), 8.28 (d, J=9.2 Hz, 1H), 8.98 (s, 1H), 11.13 (s, 1H).

MS (ESI⁺) m/z: 546 (MH⁺).

HRMS (ESI⁺) for C₃₀H₃₆N₅O₅ (MH⁺): calcd, 546.27164. found, 546.27192.

### Example 3

### 6-((4-(2-(3-Amino-6-methoxy-1,5-naphthyridin-4-yl)ethyl)bicyclo[2.2.2]octan-1-ylamino)methyl)-2H-pyrido[3,2-b][1,4]oxazin-3(4H)-one Hydrochloride

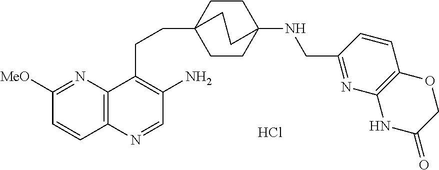 [SMILES: COc1ccc2ncc(N)c(CCC34CCC(NCc5ccc6c(n5)NC(=O)CO6)(CC3)CC4)c2n1 ; Cl]

### Step 1

### tert-Butyl 4-(2-(6-Methoxy-3-(3-tert-butoxycarbonylamino)-1,5-naphthyridin-4-yl)ethyl)bicyclo[2.2.2]octan-1-ylcarbamate

The title compound tert-butyl 4-(2-(6-methoxy-3-(3-tert-butoxycarbonylamino)-1,5-naphthyridin-4-yl)ethyl)bicyclo[2.2.2]octan-1-ylcarbamate (195 mg) was prepared from K (197 mg) and C (300 mg) in the same manner as described for Step 1 of EXAMPLE 1.

¹H NMR (CDCl₃): δ 1.35 (m, 2H), 1.43 (s, 9H), 1.55 (s, 9H), 1.59-1.70 (m, 6H), 1.78-1.98 (m, 6H), 2.98-3.11 (m, 2H), 4.05 (s, 3H), 4.34 (s, 1H), 6.27 (s, 1H), 7.02 (d, J=9.2 Hz, 1H), 8.14 (d, J=8.6 Hz, 1H), 9.06 (s, 1H).

MS (ESI⁺) m/z: 527 (MH⁺).

HRMS (ESI⁺) for C₂₉H₄₃N₄O₅ (MH⁺): calcd, 527.32334. found, 527.32337.

### Step 2

### 4-(2-(4-Aminobicyclo[2.2.2]octan-1-yl)ethyl)-6-methoxy-1,5-naphthyridin-3-amine

The title compound 4-(2-(4-aminobicyclo[2.2.2]octan-1-yl)ethyl)-6-methoxy-1,5-naphthyridin-3-amine (89.1 mg) was prepared from tert-butyl 4-(2-(6-methoxy-3-(3-tert-butoxycarbonylamino)-1,5-naphthyridin-4-yl)ethyl)bicyclo[2.2.2]octan-1-ylcarbamate (168 mg) in the same manner as described for Step 2 of EXAMPLE 1.

¹H NMR (CDCl₃): δ 0.95-1.75 (m, 16H), 2.94-2.98 (m, 2H), 3.87 (s, 2H), 4.05 (s, 3H), 6.85 (d, J=9.2 Hz, 1H), 8.02 (d, J=4.9 Hz, 1H), 8.30 (s, 1H).

MS (ESI⁺) m/z: 327 (MH⁺).

HRMS (ESI⁺) for C₁₉H₂₇N₄O (MH⁺): calcd, 327.21849. found, 327.21885.

### Step 3

### 6-((4-(2-(3-Amino-6-methoxy-1,5-naphthyridin-4-yl)ethyl)bicyclo[2.2.2]octan-1-ylamino)methyl)-2H-pyrido[3,2-b][1,4]oxazin-3(4H)-one

The title compound 6-((4-(2-(3-amino-6-methoxy-1,5-naphthyridin-4-yl)ethyl)bicyclo[2.2.2]octan-1-ylamino)methyl)-2H-pyrido[3,2-b][1,4]oxazin-3(4H)-one (93.2 mg) was prepared from 4-(2-(4-aminobicyclo[2.2.2]octan-1-yl)ethyl)-6-methoxy-1,5-naphthyridin-3-amine (83.0 mg) and I (50.0 mg) in the same manner as described for Step 3 of EXAMPLE 1.

¹H NMR (DMSO-d₆): δ 1.34-1.74 (m, 16H), 2.93-3.02 (m, 2H), 3.74 (s, 2H), 3.88 (s, 2H), 4.05 (s, 3H), 4.62 (s, 2H), 6.86 (d, J=8.6 Hz, 1H), 6.94 (d, J=7.9 Hz, 1H), 7.19 (d, J=7.9 Hz, 1H), 8.03 (d, J=9.2 Hz, 1H), 8.30 (s, 1H).

MS (ESI⁺) m/z: 489 (MH⁺).

HRMS (ESI⁺) for C₂₇H₃₃N₆O₃ (MH⁺): calcd, 489.26141. found, 489.26154.

### Step 4

### 6-((4-(2-(3-Amino-6-methoxy-1,5-naphthyridin-4-yl)ethyl)bicyclo[2.2.2]octan-1-ylamino)methyl)-2H-pyrido[3,2-b][1,4]oxazin-3(4H)-one Hydrochloride

To a solution of 6-((4-(2-(3-amino-6-methoxy-1,5-naphthyridin-4-yl)ethyl)bicyclo[2.2.2]octan-1-ylamino)methyl)-2H-pyrido[3,2-b][1,4]oxazin-3(4H)-one (90.0 mg) in dichloromethane/ethanol (5:1, 15.8 mL) was added a solution of hydrogen chloride (46 μL, 4 M in 1,4-dioxane), the mixture was stirred at room temperature for 4 hours and then concentrated in vacuo to give 6-((4-(2-(3-amino-6-methoxy-1,5-naphthyridin-4-yl)ethyl)bicyclo[2.2.2]octan-1-ylamino)methyl)-2H-pyrido[3,2-b][1,4]oxazin-3(4H)-one hydrochloride (95.4 mg).

¹H NMR (DMSO-d₆): δ 1.25-1.34 (m, 2H), 1.64 (m, 6H), 1.84 (m, 6H), 2.81-2.91 (m, 2H), 3.96 (s, 3H), 4.06 (s, 2H), 4.68 (s, 2H), 5.57 (s, 2H), 6.79 (d, J=9.2 Hz, 1H), 7.20 (d, J=7.9 Hz, 1H), 7.45 (d, J=7.9 Hz, 1H), 7.95 (d, J=8.6 Hz, 1H), 8.28 (s, 1H), 8.87 (s, 2H), 11.31 (s, 1H).

MS (ESI⁺) m/z: 489 (MH⁺) (as free base).

HRMS (ESI⁺) for C₂₇H₃₃N₆O₃ (MH⁺) (as free base): calcd, 489.26141. found, 489.26196.

### Example 4

### 6-((4-(2-(3-Fluoro-6-methoxy-1,5-naphthyridin-4-yl)ethyl)bicyclo[2.2.2]octan-1-ylamino)methyl)-2H-pyrido[3,2-b][1,4]oxazin-3(4H)-one Hydrochloride

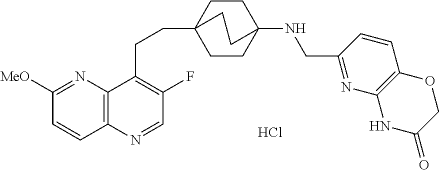 [SMILES: COc1ccc2ncc(F)c(CCC34CCC(NCc5ccc6c(n5)NC(=O)CO6)(CC3)CC4)c2n1 ; Cl]

### Step 1

### tert-Butyl 4-(2-(3-Fluoro-6-methoxy-1,5-naphthyridin-4-yl)ethyl)bicyclo[2.2.2]octan-1-ylcarbamate

The title compound tert-butyl 4-(2-(3-fluoro-6-methoxy-1,5-naphthyridin-4-yl)ethyl)bicyclo[2.2.2]octan-1-ylcarbamate (138 mg) was prepared from L (190 mg) and C (375 mg) in the same manner as described for Step 1 of EXAMPLE 1.

¹H NMR (CDCl₃): δ 1.36-1.50 (m, 11H), 1.58-1.62 (m, 6H), 1.77-1.96 (m, 6H), 3.03-3.12 (m, 2H), 4.06 (s, 3H), 4.34 (s, 1H), 7.05 (d, J=9.2 Hz, 1H), 8.15 (d, J=8.6 Hz, 1H), 8.58 (s, 1H).

MS (EI⁺) m/z: 429 (M⁺).

HRMS (EI⁺) for C₂₄H₃₂FN₃O₃ (M⁺): calcd, 429.2428. found, 429.2451.

### Step 2

### 4-(2-(3-Fluoro-6-methoxy-1,5-naphthyridin-4-yl)ethyl)bicyclo[2.2.2]octan-1-amine

The title compound 4-(2-(3-fluoro-6-methoxy-1,5-naphthyridin-4-yl)ethyl)bicyclo[2.2.2]octan-1-amine (112 mg) was prepared from tert-butyl 4-(2-(3-fluoro-6-methoxy-1,5-naphthyridin-4-yl)ethyl)bicyclo[2.2.2]octan-1-ylcarbamate (168 mg) in the same manner as described for Step 2 of EXAMPLE 1.

¹H NMR (CDCl₃): δ 0.95-1.40 (m, 2H), 1.41-1.50 (m, 2H), 1.55-1.67 (m, 12H), 3.04-3.12 (m, 2H), 4.08 (s, 3H), 7.06 (d, J=9.1 Hz, 1H), 8.15 (d, J=9.1 Hz, 1H), 8.58 (s, 1H).

MS (EI⁺) m/z: 329 (M⁺).

HRMS (EI⁺) for C₁₉H₂₄FN₃O (M⁺): calcd, 329.1903. found, 329.1919.

### Step 3

### 6-((4-(2-(3-Fluoro-6-methoxy-1,5-naphthyridin-4-yl)ethyl)bicyclo[2.2.2]octan-1-ylamino)methyl)-2H-pyrido[3,2-b][1,4]oxazin-3(4H)-one

The title compound 6-((4-(2-(3-fluoro-6-methoxy-1,5-naphthyridin-4-yl)ethyl)bicyclo[2.2.2]octan-1-ylamino)methyl)-2H-pyrido[3,2-b][1,4]oxazin-3(4H)-one (120 mg) was prepared from 4-(2-(3-fluoro-6-methoxy-1,5-naphthyridin-4-yl)ethyl)bicyclo[2.2.2]octan-1-amine (100 mg) and I (59.3 mg) in the same manner as described for Step 3 of EXAMPLE 1.

¹H NMR (DMSO-d₆): δ 1.36-1.44 (m, 2H), 1.50-1.61 (m, 13H), 2.98-3.07 (m, 2H), 3.60 (s, 2H), 4.03 (s, 3H), 4.58 (s, 2H), 7.01 (d, J=7.9 Hz, 1H), 7.22 (d, J=9.1 Hz, 1H), 7.27 (d, J=8.5 Hz, 1H), 8.26 (d, J=9.1 Hz, 1H), 8.74 (s, 1H), 11.14 (s, 1H).

MS (ESI⁺) m/z: 492 (MH⁺).

HRMS (ESI⁺) for C₂₇H₃₁FN₅O₃ (MH⁺): calcd, 492.24109. found, 492.24062.

### Step 4

### 6-((4-(2-(3-Fluoro-6-methoxy-1,5-naphthyridin-4-yl)ethyl)bicyclo[2.2.2]octan-1-ylamino)methyl)-2H-pyrido[3,2-b][1,4]oxazin-3(4H)-one Hydrochloride

The title compound 6-((4-(2-(3-fluoro-6-methoxy-1,5-naphthyridin-4-yl)ethyl)bicyclo[2.2.2]octan-1-ylamino)methyl)-2H-pyrido[3,2-b][1,4]oxazin-3(4H)-one hydrochloride (87.5 mg) was prepared from 6-((4-(2-(3-fluoro-6-methoxy-1,5-naphthyridin-4-yl)ethyl)bicyclo[2.2.2]octan-1-ylamino)methyl)-2H-pyrido[3,2-b][1,4]oxazin-3(4H)-one (109 mg) in the same manner as described for Step 4 of EXAMPLE 3.

¹H NMR (DMSO-d₆): δ 1.39-1.50 (m, 2H), 1.56-1.72 (m, 6H), 1.79-1.98 (m, 6H), 2.98-3.09 (m, 2H), 4.04 (s, 5H), 4.68 (s, 2H), 7.20-7.25 (m, 2H), 7.45 (d, J=7.9 Hz, 1H), 8.27 (d, J=9.2 Hz, 1H), 8.76 (s, 1H), 8.92 (s, 2H), 11.31 (s, 1H).

MS (ESI⁺) m/z: 492 (MH⁺) (as free base).

HRMS (ESI⁺) for C₂₇H₃₁FN₅O₃ (MH⁺) (as free base): calcd, 492.24109. found, 492.24095.

### Example 5

### 6-((4-(2-(3-Chloro-6-methoxy-1,5-naphthyridin-4-yl)ethyl)bicyclo[2.2.2]octan-1-ylamino)methyl)-2H-pyrido[3,2-b][1,4]oxazin-3(4H)-one Hydrochloride

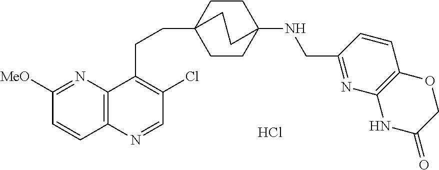 [SMILES: COc1ccc2ncc(Cl)c(CCC34CCC(NCc5ccc6c(n5)NC(=O)CO6)(CC3)CC4)c2n1 ; Cl]

### Step 1

### tert-Butyl 4-(2-(3-Chloro-6-methoxy-1,5-naphthyridin-4-yl)ethyl)bicyclo[2.2.2]octan-1-ylcarbamate

The title compound tert-butyl 4-(2-(3-chloro-6-methoxy-1,5-naphthyridin-4-yl)ethyl)bicyclo[2.2.2]octan-1-ylcarbamate (1.64 g) was prepared from M (1.52 g) and C (2.00 g) in the same manner as described for Step 1 of EXAMPLE 1.

¹H NMR (CDCl₃): δ 1.36-1.47 (m, 11H), 1.62-1.68 (m, 6H), 1.81-1.92 (m, 6H), 3.17-3.26 (m, 2H), 4.06 (s, 3H), 4.34 (br, 1H), 7.09 (d, J=9.2 Hz, 1H), 8.14 (d, J=9.2 Hz, 1H), 8.63 (s, 1H).

MS (ESI⁺) m/z: 446 (MH⁺).

HRMS (ESI⁺) for C₂₄H₃₃ClN₃O₃ (MH⁺): calcd, 446.22104. found, 446.22132.

### Step 2

### 4-(2-(3-Chloro-6-methoxy-1,5-naphthyridin-4-yl)ethyl)bicyclo[2.2.2]octan-1-amine

The title compound 4-(2-(3-chloro-6-methoxy-1,5-naphthyridin-4-yl)ethyl)bicyclo[2.2.2]octan-1-amine (152 mg) was prepared from tert-butyl 4-(2-(3-chloro-6-methoxy-1,5-naphthyridin-4-yl)ethyl)bicyclo[2.2.2]octan-1-ylcarbamate (200 mg) in the same manner as described for Step 2 of EXAMPLE 1.

¹H NMR (DMSO-d₆): δ 1.30-1.36 (m, 2H), 1.43-1.59 (m, 12H), 3.12-3.16 (m, 2H), 4.02 (s, 3H), 7.26 (d, J=9.2 Hz, 1H), 8.25 (d, J=9.2 Hz, 1H), 8.71 (s, 1H).

MS (ESI⁺) m/z: 346 (MH⁺).

HRMS (ESI⁺) for C₁₉H₂₅ClN₃O (MH⁺): calcd, 346.16861. found, 346.16896.

### Step 3

### 6-((4-(2-(3-Chloro-6-methoxy-1,5-naphthyridin-4-yl)ethyl)bicyclo[2.2.2]octan-1-ylamino)methyl)-2H-pyrido[3,2-b][1,4]oxazin-3(4H)-one

The title compound 6-((4-(2-(3-chloro-6-methoxy-1,5-naphthyridin-4-yl)ethyl)bicyclo[2.2.2]octan-1-ylamino)methyl)-2H-pyrido[3,2-b][1,4]oxazin-3(4H)-one (109 mg) was prepared from 4-(2-(3-chloro-6-methoxy-1,5-naphthyridin-4-yl)ethyl)bicyclo[2.2.2]octan-1-amine (140 mg) and I (79.3 mg) in the same manner as described for Step 3 of EXAMPLE 1.

¹H NMR (DMSO-d₆): δ 1.30-1.38 (m, 2H), 1.56 (m, 12H), 3.12-3.20 (m, 2H), 3.62 (s, 2H), 4.03 (s, 3H), 4.59 (s, 2H), 7.01 (d, J=7.9 Hz, 1H), 7.27 (d, J=9.2 Hz, 2H), 8.26 (d, J=9.2 Hz, 1H), 8.72 (s, 1H), 11.14 (br, 1H).

MS (ESI⁺) m/z: 508 (WO.

HRMS (ESI⁺) for C₂₇H₃₁ClN₅O₃ (MH⁺): calcd, 508.21154. found, 508.21154.

### Step 4

### 6-((4-(2-(3-Chloro-6-methoxy-1,5-naphthyridin-4-yl)ethyl)bicyclo[2.2.2]octan-1-ylamino)methyl)-2H-pyrido[3,2-b][1,4]oxazin-3(4H)-one Hydrochloride

The title compound 6-((4-(2-(3-chloro-6-methoxy-1,5-naphthyridin-4-yl)ethyl)bicyclo[2.2.2]octan-1-ylamino)methyl)-2H-pyrido[3,2-b][1,4]oxazin-3(4H)-one hydrochloride (90.2 mg) was prepared from 6-((4-(2-(3-chloro-6-methoxy-1,5-naphthyridin-4-yl)ethyl)bicyclo[2.2.2]octan-1-ylamino)methyl)-2H-pyrido[3,2-b][1,4]oxazin-3(4H)-one (87.0 mg) in the same manner as described for Step 4 of EXAMPLE 3.

¹H NMR (DMSO-d₆): δ 1.34-1.43 (m, 2H), 1.58-1.72 (m, 6H), 1.78-1.96 (m, 6H), 3.13-3.22 (m, 2H), 3.99-4.10 (br, 2H), 4.04 (s, 3H), 4.68 (s, 2H), 7.17-7.25 (m, 1H), 7.29 (d, J=9.2 Hz, 1H), 7.45 (d, J=8.6 Hz, 1H), 8.27 (d, J=9.2 Hz, 1H), 8.74 (s, 1H), 8.94 (br, 2H), 11.32 (br, 1H).

MS (ESI⁺) m/z: 508 (MH⁺) (as free base).

HRMS (ESI⁺) for C₂₇H₃₁ClN₅O₃ (MH⁺) (as free base): calcd, 508.21154. found, 508.21072.

### Example 6

### 6-((4-((3-Chloro-6-methoxy-1,5-naphthyridin-4-yl)ethynyl)bicyclo[2.2.2]octan-1-ylamino)methyl)-2H-pyrido[3,2-b][1,4]oxazin-3(4H)-one Hydrochloride

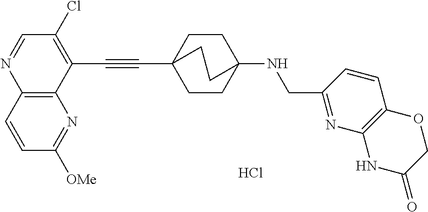 [SMILES: COc1ccc2ncc(Cl)c(C#CC34CCC(NCc5ccc6c(n5)NC(=O)CO6)(CC3)CC4)c2n1 ; Cl]

### Step 1

### tert-Butyl 4-((3-Chloro-6-methoxy-1,5-naphthyridin-4-yl)ethynyl)bicyclo[2.2.2]octan-1-ylcarbamate

A degassed mixture of D (3.50 g), M (2.56 g) and copper(I) iodide (534 mg) in N,N-dimethylformamide (93.5 mL) was added bis(triphenylphosphine)palladium(II) dichloride (985 mg) and triethylamine (19.5 mL), the mixture was stirred at 60° C. for overnight and then concentrated in vacuo. After dilution of the residue with ethyl acetate, the mixture was washed with water and brine, dried over anhydrous sodium sulfate, filtered, and then concentrated in vacuo. Flash chromatography (silica, hexane:ethyl acetate=4:1) of the residue gave tert-butyl 4-((3-chloro-6-methoxy-1,5-naphthyridin-4-yl)ethynyl)bicyclo[2.2.2]octan-1-ylcarbamate (2.24 g).

¹H NMR (CDCl₃): δ 1.43 (s, 9H), 1.91-1.97 (m, 6H), 2.03-2.12 (m, 6H), 4.12 (s, 3H), 4.35 (br, 1H), 7.10 (d, J=9.2 Hz, 1H), 8.14 (d, J=8.6 Hz, 1H), 8.69 (s, 1H).

### Step 2

### 4-((3-Chloro-6-methoxy-1,5-naphthyridin-4-yl)ethynyl)bicyclo[2.2.2]octan-1-amine

The title compound 4-((3-chloro-6-methoxy-1,5-naphthyridin-4-yl)ethynyl)bicyclo[2.2.2]octan-1-amine (340 mg) was prepared from tert-butyl 4-((3-chloro-6-methoxy-1,5-naphthyridin-4-yl)ethynyl)bicyclo[2.2.2]octan-1-ylcarbamate (450 mg) in the same manner as described for Step 2 of EXAMPLE 1.

¹H NMR (DMSO-d₆): δ 1.35 (br, 2H), 1.45-1.52 (m, 6H), 1.89-1.96 (m, 6H), 4.05 (s, 1H), 7.29 (d, J=9.2 Hz, 1H), 8.27 (d, J=9.2 Hz, 1H), 8.81 (s, 1H).

MS (ESI⁺) m/z: 342 (MH⁺).

HRMS (ESI⁺) for C₁₉H₂₁ClN₃O (MH⁺): calcd, 342.13731. found, 342.13694.

### Step 3

### 6-((4-((3-Chloro-6-methoxy-1,5-naphthyridin-4-yl)ethynyl)bicyclo[2.2.2]octan-1-ylamino)methyl)-2H-pyrido[3,2-b][1,4]oxazin-3(4H)-one

The title compound 6-((4-((3-chloro-6-methoxy-1,5-naphthyridin-4-yl)ethynyl)bicyclo[2.2.2]octan-1-ylamino)methyl)-2H-pyrido[3,2-b][1,4]oxazin-3(4H)-one (150 mg) was prepared from 4-((3-chloro-6-methoxy-1,5-naphthyridin-4-yl)ethynyl)bicyclo[2.2.2]octan-1-amine (300 mg) and I (172 mg) in the same manner as described for Step 3 of EXAMPLE 1.

¹H NMR (DMSO-d₆): δ 1.52-1.64 (m, 6H), 1.76 (br, 1H), 1.90-2.02 (m, 6H), 3.61 (brs, 2H), 4.05 (s, 3H), 4.58 (s, 2H), 7.02 (d, J=7.9 Hz, 1H), 7.27 (d, J=8.6 Hz, 1H), 7.29 (d, J=9.2 Hz, 1H), 8.28 (d, J=9.2 Hz, 1H), 8.82 (s, 1H), 11.14 (br, 1H).

MS (ESI⁺) m/z: 504 (MH⁺).

HRMS (ESI⁺) for C₂₇H₂₇ClN₅O₃ (MH⁺): calcd, 504.18024. found, 504.18010.

### Step 4

### 6-((4-((3-Chloro-6-methoxy-1,5-naphthyridin-4-yl)ethynyl)bicyclo[2.2.2]octan-1-ylamino)methyl)-2H-pyrido[3,2-b][1,4]oxazin-3(4H)-one Hydrochloride

The title compound 6-((4-((3-chloro-6-methoxy-1,5-naphthyridin-4-yl)ethynyl)bicyclo[2.2.2]octan-1-ylamino)methyl)-2H-pyrido[3,2-b][1,4]oxazin-3(4H)-one hydrochloride (107 mg) was prepared from 6-((4-((3-chloro-6-methoxy-1,5-naphthyridin-4-yl)ethynyl)bicyclo[2.2.2]octan-1-ylamino)methyl)-2H-pyrido[3,2-b][1,4]oxazin-3(4H)-one (100 mg) in the same manner as described for Step 4 of EXAMPLE 3.

¹H NMR (DMSO-d₆): δ 1.87-2.01 (m, 6H), 2.05-2.12 (m, 6H), 4.07 (s, 3H), 4.69 (s, 2H), 7.21 (d, J=7.9 Hz, 1H), 7.31 (d, J=9.2 Hz, 1H), 7.45 (d, J=8.6 Hz, 1H), 8.30 (d, J=9.2 Hz, 1H), 8.84 (s, 1H), 9.04 (br, 2H), 11.33 (br, 1H).

MS (ESI⁺) m/z: 504 (MH⁺).

HRMS (ESI⁺) for C₂₇H₂₇ClN₅O₃ (MH⁺): calcd, 504.18024. found, 504.18010.

### Example 7

### (Z)-6-((4-(2-(3-Chloro-6-hydroxy-1,5-naphthyridin-4-yl)vinyl)bicyclo[2.2.2]octan-1-ylamino)methyl)-2H-pyrido[3,2-b][1,4]oxazin-3(4H)-one

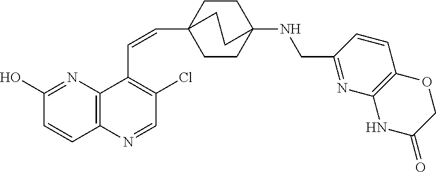 [SMILES: O=C1COc2ccc(CNC34CCC(/C=C\c5c(Cl)cnc6ccc(O)nc56)(CC3)CC4)nc2N1]

### Step 1

### Methyl 4-((3-Chloro-6-methoxy-1,5-naphthyridin-4-yl)ethynyl)bicyclo[2.2.2]octane-1-carboxylate

The title compound methyl 4-((3-chloro-6-methoxy-1,5-naphthyridin-4-yl)ethynyl)bicyclo[2.2.2]octane-1-carboxylate (29.4 mg) was prepared from M (31.6 mg) and N (30.0 mg) in the same manner as described for Step 1 of EXAMPLE 6.

¹H NMR (CDCl₃): δ 1.81-1.91 (m, 6H), 1.97-2.06 (m, 6H), 3.67 (s, 3H), 4.13 (s, 3H), 7.11 (d, J=9.2 Hz, 1H), 8.15 (d, J=9.2 Hz, 1H), 8.70 (s, 1H).

MS (ESI⁺) m/z: 385 (MH⁺).

HRMS (ESI⁺) for C₂₁H₂₂ClN₂O₃ (MH⁺): calcd, 385.13189. found, 385.13231.

### Step 2

### (Z)-Methyl 4-(2-(3-Chloro-6-methoxy-1,5-naphthyridin-4-yl)vinyl)bicyclo[2.2.2]octane-1-carboxylate

A suspension of 4-((3-chloro-6-methoxy-1,5-naphthyridin-4-yl)ethynyl)bicyclo[2.2.2]octane-1-carboxylate (200 mg) and 5% platinum on carbon (88.9 mg) in tetrahydrofuran (29 mL) was stirred at room temperature for 7 hours under H₂ atmosphere (3 kg/cm²). After the insoluble materials were filtered off, the filtrate was concentrated in vacuo. Flash chromatography (silica, toluene:acetonitrile=20:1) of the residue gave (Z)-methyl 4-(2-(3-chloro-6-methoxy-1,5-naphthyridin-4-yl)vinyl)bicyclo[2.2.2]octane-1-carboxylate (126 mg).

¹H NMR (CDCl₃): δ 1.32-1.51 (m, 6H), 1.53-1.66 (m, 6H), 3.57 (s, 3H), 4.05 (s, 3H), 5.76 (d, J=13.4 Hz, 1H), 6.18 (d, J=12.8 Hz, 1H), 7.10 (d, J=9.2 Hz, 1H), 8.17 (d, J=9.2 Hz, 1H), 8.69 (s, 1H).

MS (ESI⁺) m/z: 387 (MH⁺).

HRMS (ESI⁺) for C₂₁H₂₄ClN₂O₃ (MH⁺): calcd, 387.14754. found, 387.14761.

### Step 3

### (Z)-4-(2-(3-Chloro-6-methoxy-1,5-naphthyridin-4-yl)vinyl)bicyclo[2.2.2]octane-1-carboxylic Acid

The title compound (Z)-4-(2-(3-chloro-6-methoxy-1,5-naphthyridin-4-yl)vinyl)bicyclo[2.2.2]octane-1-carboxylic acid was prepared from (Z)-methyl 4-(2-(3-chloro-6-methoxy-1,5-naphthyridin-4-yl)vinyl)bicyclo[2.2.2]octane-1-carboxylate (60.0 mg) in the same manner as described for Step 2 of EXAMPLE 15.

MS (ESI⁺) m/z: 373 (MH⁺).

HRMS (ESI⁺): for C₂₀H₂₂ClN₂O₃ (MH⁺): calcd, 373.13189. found, 373.13162.

### Step 4

### (Z)-4-(2-(3-Chloro-6-hydroxy-1,5-naphthyridin-4-yl)vinyl)bicyclo[2.2.2]octan-1-amine

The title compound (Z)-4-(2-(3-chloro-6-hydroxy-1,5-naphthyridin-4-yl)vinyl)bicyclo[2.2.2]octan-1-amine (43.5 mg) was prepared from (Z)-methyl 4-(2-(3-chloro-6-methoxy-1,5-naphthyridin-4-yl)vinyl)bicyclo[2.2.2]octane-1-carboxylate (48.0 mg) in the same manner as described for Step 3 of EXAMPLE 15.

¹H NMR (DMSO-d₆): δ 1.36-1.60 (m, 12H), 5.82 (d, J=12.8 Hz, 1H), 6.02 (d, J=12.8 Hz, 1H), 6.77 (d, J=9.8 Hz, 1H), 7.72-7.75 (br, 3H), 7.94 (d, J=9.8 Hz, 1H), 8.51 (s, 1H).

MS (ESI⁺) m/z: 330 (MH⁺).

### Step 5

### (Z)-6-((4-(2-(3-Chloro-6-hydroxy-1,5-naphthyridin-4-yl)vinyl)bicyclo[2.2.2]octan-1-ylamino)methyl)-2H-pyrido[3,2-b][1,4]oxazin-3(4H)-one

The title compound (Z)-6-((4-(2-(3-chloro-6-hydroxy-1,5-naphthyridin-4-yl)vinyl)bicyclo[2.2.2]octan-1-ylamino)methyl)-2H-pyrido[3,2-b][1,4]oxazin-3(4H)-one (21.6 mg) was prepared from (Z)-4-(2-(3-chloro-6-hydroxy-1,5-naphthyridin-4-yl)vinyl)bicyclo[2.2.2]octan-1-amine (35.0 mg) and I (18.1 mg) in the same manner as described for Step 3 of EXAMPLE 1.

¹H NMR (DMSO-d₆): δ 1.28-1.49 (m, 12H), 3.49 (s, 2H), 4.55 (s, 2H), 5.83 (d, J=12.8 Hz, 1H), 5.97 (d, J=12.8 Hz, 1H), 6.77 (d, J=9.8 Hz, 1H), 6.93 (d, J=7.9 Hz, 1H), 7.22 (d, J=7.9 Hz, 1H), 7.94 (d, J=9.8 Hz, 1H), 8.50 (s, 1H).

MS (ESI⁺) m/z: 492 (MH⁺).

HRMS (ESI⁺) for C₂₆H₂₇ClN₅O₃ (MH⁺): calcd, 492.18024. found, 492.18023.

### Example 8

### (Z)-6-((4-(2-(3-Chloro-6-methoxy-1,5-naphthyridin-4-yl)vinyl)bicyclo[2.2.2]octan-1-ylamino)methyl)-2H-pyrido[3,2-b][1,4]oxazin-3(4H)-one

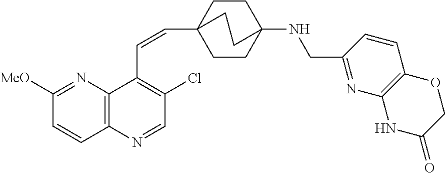 [SMILES: COc1ccc2ncc(Cl)c(/C=C\C34CCC(NCc5ccc6c(n5)NC(=O)CO6)(CC3)CC4)c2n1]

### Step 1

### (Z)-4-(2-(3-Chloro-6-methoxy-1,5-naphthyridin-4-yl)vinyl)bicyclo[2.2.2]octan-1-amine

A mixture of (Z)-4-(2-(3-chloro-6-methoxy-1,5-naphthyridin-4-yl)vinyl)bicyclo[2.2.2]octane-1-carboxylic acid (40.0 mg), triethylamine (15.8 μL) and diphenyl phosphoryl azide (24.5 μL) in toluene (1 mL) was stirred at room temperature for 2 hours, reflux at 120° C. and concentrated in vacuo. A solution of the residue in 1,4-dioxane (0.53 mL) and 6 N hydrochloric acid (0.53 mL) was stirred at room temperature for 1 hour. After dilution of the residue with dichloromethane and water, the mixture was washed with 1 N sodium hydroxide solution and brine, dried over anhydrous sodium sulfate, filtered, and then concentrated in vacuo to give (Z)-4-(2-(3-chloro-6-methoxy-1,5-naphthyridin-4-yl)vinyl)bicyclo[2.2.2]octan-1-amine (35.6 mg).

¹H NMR (CDCl₃): δ 1.25-1.78 (m, 12H), 4.04 (s, 3H), 5.76 (d, J=12.8 Hz, 1H), 6.16 (d, J=13.4 Hz, 1H), 7.10 (d, J=9.2 Hz, 1H), 8.17 (d, J=9.2 Hz, 1H), 8.69 (s, 1H).

### Step 2

### (Z)-6-((4-(2-(3-Chloro-6-methoxy-1,5-naphthyridin-4-yl)vinyl)bicyclo[2.2.2]octan-1-ylamino)methyl)-2H-pyrido[3,2-b][1,4]oxazin-3(4H)-one

The title compound (Z)-6-((4-(2-(3-chloro-6-methoxy-1,5-naphthyridin-4-yl)vinyl)bicyclo[2.2.2]octan-1-ylamino)methyl)-2H-pyrido[3,2-b][1,4]oxazin-3(4H)-one (20.7 mg) was prepared from (Z)-4-(2-(3-chloro-6-methoxy-1,5-naphthyridin-4-yl)vinyl)bicyclo[2.2.2]octan-1-amine (35.5 mg) and I (18.4 mg) in the same manner as described for Step 3 of EXAMPLE 1.

¹H NMR (DMSO-d₆): δ 1.26-1.55 (m, 12H), 3.46 (s, 2H), 3.97 (s, 3H), 4.55 (s, 2H), 5.77 (d, J=12.8 Hz, 1H), 6.18 (d, J=12.8 Hz, 1H), 6.91 (d, J=7.9 Hz, 1H), 7.21 (d, J=7.9 Hz, 1H), 7.27 (d, J=9.2 Hz, 1H), 8.28 (d, J=8.6 Hz, 1H), 8.78 (s, 1H).

MS (ESI⁺) m/z: 506 (MH⁺).

HRMS (ESI⁺) for C₂₇H₂₉ClN₅O₃ (MH⁺): calcd, 506.19589. found, 506.19554.

### Example 9

### 6-((4-(2-(3-Chloro-6-hydroxy-1,5-naphthyridin-4-yl)ethyl)bicyclo[2.2.2]octan-1-ylamino)methyl)-2H-pyrido[3,2-b][1,4]oxazin-3(4H)-one

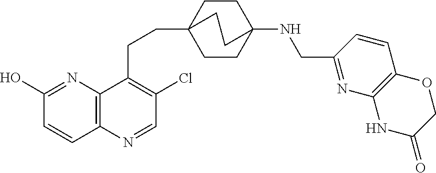 [SMILES: O=C1COc2ccc(CNC34CCC(CCc5c(Cl)cnc6ccc(O)nc56)(CC3)CC4)nc2N1]

A solution of 6-((4-(2-(3-chloro-6-methoxy-1,5-naphthyridin-4-yl)ethyl)bicyclo[2.2.2]octan-1-ylamino)methyl)-2H-pyrido[3,2-b][1,4]oxazin-3(4H)-one (145 mg) in 6 M hydrochloric acid (3.0 mL) was stirred under reflux for 1.5 hours and concentrated in vacuo. Treatment of the residue with water gave 6-((4-(2-(3-chloro-6-hydroxy-1,5-naphthyridin-4-yl)ethyl)bicyclo[2.2.2]octan-1-ylamino)methyl)-2H-pyrido[3,2-b][1,4]oxazin-3(4H)-one (78.3 mg).

¹H NMR (DMSO-d₆): δ 1.20-1.24 (m, 2H), 1.54 (s, 12H), 2.90-3.00 (m, 2H), 3.65 (s, 2H), 4.59 (s, 2H), 6.76 (d, J=9.8 Hz, 1H), 7.02 (d, J=7.9 Hz, 1H), 7.28 (d, J=8.6 Hz, 1H), 7.91 (d, J=9.8 Hz, 1H), 8.44 (s, 1H), 11.15 (br, 1H).

MS (ESI⁺) m/z: 494 (MH⁺).

HRMS (ESI⁺) for C₂₆H₂₉ClN₅O₃ (MH⁺): calcd, 494.19589. found, 494.19561.

### Example 10

### (E)-6-((4-(2-(3-Chloro-6-methoxy-1,5-naphthyridin-4-yl)vinyl)bicyclo[2.2.2]octan-1-ylamino)methyl)-2H-pyrido[3,2-b][1,4]oxazin-3(4H)-one Hydrochloride

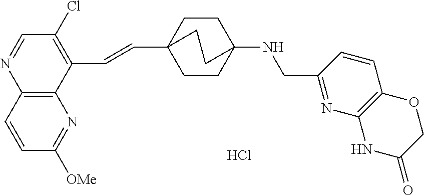 [SMILES: COc1ccc2ncc(Cl)c(/C=C/C34CCC(NCc5ccc6c(n5)NC(=O)CO6)(CC3)CC4)c2n1 ; Cl]

### Step 1

### (E)-tert-Butyl 4-(2-(3-Chloro-6-methoxy-1,5-naphthyridin-4-yl)vinyl)bicyclo[2.2.2]octan-1-ylcarbamate

The title compound (E)-tert-butyl 4-(2-(3-chloro-6-methoxy-1,5-naphthyridin-4-yl)vinyl)bicyclo[2.2.2]octan-1-ylcarbamate (1.87 g) was prepared from E (3.20 g) and M (2.45 g) in the same manner as described for Step 1 of EXAMPLE 1.

¹H NMR (DMSO-d₆): δ 1.36 (s, 9H), 1.63-1.75 (m, 6H), 1.77-1.84 (m, 6H), 4.00 (s, 3H), 6.42 (br, 1H), 6.69 (d, J=16.5 Hz, 1H), 7.28 (d, J=9.2 Hz, 1H), 7.39 (d, J=16.5 Hz, 1H), 8.26 (d, J=9.2 Hz, 1H), 8.74 (s, 1H).

MS (ESI⁺) m/z: 444 (MH⁺).

HRMS (ESI⁺) for C₂₄H₃₁ClN₃O₃ (MH⁺): calcd, 444.20539. found, 444.20515.

### Step 2

### (E)-4-(2-(3-Chloro-6-methoxy-1,5-naphthyridin-4-yl)vinyl)bicyclo[2.2.2]octan-1-amine

The title compound (E)-4-(2-(3-chloro-6-methoxy-1,5-naphthyridin-4-yl)vinyl)bicyclo[2.2.2]octan-1-amine (337 mg) was prepared from (E)-tert-butyl 4-(2-(3-chloro-6-methoxy-1,5-naphthyri din-4-yl)vinyl)bicyclo[2.2.2]octan-1-ylcarbamate (450 mg) in the same manner as described for Step 2 of EXAMPLE 1.

¹H NMR (DMSO-d₆): δ 1.55-1.61 (m, 6H), 1.69-1.76 (m, 6H), 4.00 (s, 3H), 6.69 (d, J=16.5 Hz, 1H), 7.28 (d, J=9.2 Hz, 1H), 7.40 (d, J=16.5 Hz, 1H), 8.26 (d, J=8.6 Hz, 1H), 8.74 (s, 1H).

MS (ESI⁺) m/z: 344 (MH⁺).

HRMS (ESI⁺) for C₁₉H₂₃ClN₃O (MH⁺): calcd, 344.15296. found, 344.15284.

### Step 3

### (E)-6-((4-(2-(3-Chloro-6-methoxy-1,5-naphthyridin-4-yl)vinyl)bicyclo[2.2.2]octan-1-ylamino)methyl)-2H-pyrido[3,2-b][1,4]oxazin-3(4H)-one

The title compound (E)-6-((4-(2-(3-chloro-6-methoxy-1,5-naphthyridin-4-yl)vinyl)bicyclo[2.2.2]octan-1-ylamino)methyl)-2H-pyrido[3,2-b][1,4]oxazin-3(4H)-one (297 mg) was prepared from (E)-4-(2-(3-chloro-6-methoxy-1,5-naphthyridin-4-yl)vinyl)bicyclo[2.2.2]octan-1-amine (300 mg) and I (155 mg) in the same manner as described for Step 3 of EXAMPLE 1.

¹H NMR (DMSO-d₆): δ 1.53-1.62 (m, 6H), 1.67-1.74 (m, 6H), 3.63 (s, 2H), 4.00 (s, 3H), 4.59 (s, 2H), 6.71 (d, J=16.5 Hz, 1H), 7.02 (d, J=7.9 Hz, 1H), 7.27 (d, J=8.6 Hz, 1H), 7.28 (d, J=9.2 Hz, 1H), 7.42 (d, J=16.5 Hz, 1H), 8.26 (d, J=9.2 Hz, 1H), 8.75 (s, 1H), 11.14 (br, 1H).

MS (ESI⁺) m/z: 506 (MH⁺).

HRMS (ESI⁺): calcd for C₂₇H₂₉ClN₅O₃, 506.19589. found, 506.19590.

### Step 4

### (E)-6-((4-(2-(3-Chloro-6-methoxy-1,5-naphthyridin-4-yl)vinyl)bicyclo[2.2.2]octan-1-ylamino)methyl)-2H-pyrido[3,2-b][1,4]oxazin-3(4H)-one Hydrochloride

The title compound (E)-6-((4-(2-(3-chloro-6-methoxy-1,5-naphthyridin-4-yl)vinyl)bicyclo[2.2.2]octan-1-ylamino)methyl)-2H-pyrido[3,2-b][1,4]oxazin-3(4H)-one hydrochloride (116 mg) was prepared from (E)-6-((4-(2-(3-chloro-6-methoxy-1,5-naphthyridin-4-yl)vinyl)bicyclo[2.2.2]octan-1-ylamino)methyl)-2H-pyrido[3,2-b][1,4]oxazin-3(4H)-one (100 mg) in the same manner as described for Step 4 of EXAMPLE 3.

¹H NMR (DMSO-d₆): δ 1.80-1.83 (m, 6H), 1.94-1.97 (m, 6H), 4.01 (s, 3H), 4.08 (t, J=6.7 Hz, 1H), 4.69 (s, 2H), 6.74 (d, J=16.5 Hz, 1H), 7.26 (d, J=7.9 Hz, 1H), 7.30 (d, J=9.2 Hz, 1H), 7.41 (d, J=16.5 Hz, 1H), 7.45 (d, J=7.9 Hz, 1H), 8.28 (d, J=8.6 Hz, 1H), 8.77 (s, 1H), 9.09 (br, 2H), 11.33 (s, 1H).

MS (ESI⁺) m/z: 506 (MH⁺).

HRMS (ESI⁺) for C₂₇H₂₉ClN₅O₃ (MH⁺): calcd, 506.19589. found, 506.19590.

### Example 11

### 6-((4-(4-Methylbenzo[d]thiazole-2-carbonyl)bicyclo[2.2.2]octan-1-ylamino)methyl)-2H-pyrido[3,2-b][1,4]oxazin-3(4H)-one

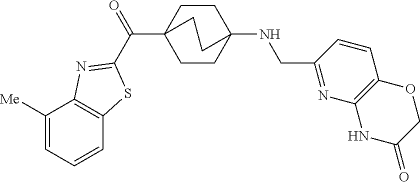 [SMILES: Cc1cccc2sc(C(=O)C34CCC(NCc5ccc6c(n5)NC(=O)CO6)(CC3)CC4)nc12]

### Step 1

### tert-Butyl 4-(Hydroxy(4-methylbenzo[d]thiazol-2-yl)methyl)bicyclo[2.2.2]octan-1-ylcarbamate

To a solution of 4-methylbenzo[d]thiazole (835 mg) in tetrahydrofuran (20 mL) was added a solution of butyllithium (2.0 mL 2.77 M in hexane) at −78° C., the mixture was stirred at the same temperature for 15 minutes. The resulting solution was added a solution of A (709 mg) in tetrahydrofuran (5.6 mL) at −78° C., the mixture was stirred at the same temperature for 50 minutes and further stirred at room temperature for 2 hours. After quenching the reaction by adding saturated ammonium chloride solution, the mixture was extracted with ethyl acetate. The organic extracts were washed with brine, dried over anhydrous sodium sulfate, filtered, and then concentrated in vacuo. Flash chromatography (silica, hexane:ethyl acetate=3:1) of the residue gave tert-butyl 4-(hydroxy(4-methylbenzo[d]thiazol-2-yl)methyl)bicyclo[2.2.2]octan-1-ylcarbamate (339 mg).

¹H NMR (CDCl₃): δ 1.41 (s, 9H), 1.68-1.77 (m, 6H), 1.77-1.88 (m, 6H), 2.72 (s, 3H), 3.10 (d, J=5.5 Hz, 1H), 4.30 (s, 1H), 4.68 (d, J=4.9 Hz, 1H), 7.26-7.30 (m, 2H), 7.69-7.71 (m, 1H).

MS (CI⁺) m/z: 403 (MH⁺).

HRMS (CI⁺) for C₂₂H₃₁N₂O₃S (MH⁺): calcd, 403.2055. found, 403.2035.

### Step 2

### tert-Butyl 4-(4-Methylbenzo[d]thiazole-2-carbonyl)bicyclo[2.2.2]octan-1-ylcarbamate

The title compound tert-butyl 4-(4-methylbenzo[d]thiazole-2-carbonyl)bicyclo[2.2.2]octan-1-ylcarbamate (232 mg) was prepared from tert-butyl 4-(hydroxy(4-methylbenzo[d]thiazol-2-yl)methyl)bicyclo[2.2.2]octan-1-ylcarbamate (300 mg) in the same manner as described for Step 9 of Intermediate A.

¹H NMR (CDCl₃): δ 1.45 (s, 9H), 1.92-2.04 (m, 6H), 2.23-2.37 (m, 6H), 2.78 (s, 3H), 4.42 (s, 1H), 7.33 (d, J=7.6 Hz, 1H), 7.39 (t, J=7.6 Hz, 1H), 7.76 (d, J=7.6 Hz, 1H).

MS (ESI⁺) m/z: 401 (MH⁺).

HRMS (ESI⁺) for C₂₂H₂₉N₂O₃S (MH⁺): calcd, 401.18989. found, 401.18907.

### Step 3

### (4-Aminobicyclo[2.2.2]octan-1-yl)(4-methylbenzo[d]thiazol-2-yl)methanone

The title compound (4-aminobicyclo[2.2.2]octan-1-yl)(4-methylbenzo[d]thiazol-2-yl)methanone (132 mg) was prepared from (200 mg) in the same manner as described for Step 2 of EXAMPLE 1.

¹H NMR (DMSO-d₆): δ 1.31 (s, 2H), 1.46-1.63 (m, 6H), 2.09-2.23 (m, 6H), 2.73 (s, 3H), 7.44 (d, J=7.3 Hz, 1H), 7.50 (t, J=7.3 Hz, 1H), 8.00 (d, J=7.3 Hz, 1H).

MS (ESI⁺) m/z: 301 (MH⁺).

HRMS (ESI⁺) for C₁₇H₂₁N₂OS (MH⁺): calcd, 301.13746. found, 301.13778.

### Step 4

### 6-((4-(4-Methylbenzo[d]thiazole-2-carbonyl)bicyclo[2.2.2]octan-1-ylamino)methyl)-2H-pyrido[3,2-b][1,4]oxazin-3(4H)-one

The title compound 6-((4-(4-methylbenzo[d]thiazole-2-carbonyl)bicyclo[2.2.2]octan-1-ylamino)methyl)-2H-pyrido[3,2-b][1,4]oxazin-3(4H)-one (47.2 mg) was prepared from (4-aminobicyclo[2.2.2]octan-1-yl)(4-methylbenzo[d]thiazol-2-yl)methanone (60.0 mg) and I (35.6 mg) in the same manner as described for Step 3 of EXAMPLE 1.

¹H NMR (DMSO-d₆): δ 1.55-1.73 (m, 6H), 2.10-2.27 (m, 6H), 2.73 (s, 3H), 3.65 (s, 2H), 4.59 (s, 2H), 7.04 (d, J=7.9 Hz, 1H), 7.28 (d, J=7.9 Hz, 1H), 7.45 (d, J=7.3 Hz, 1H), 7.50 (t, J=7.6 Hz, 1H), 8.00 (d, J=7.9 Hz, 1H), 11.15 (s, 1H).

MS (ESI⁺) m/z: 463 (MH⁺).

HRMS (ESI⁺) for C₂₅H₂₇N₄O₃S (MH⁺): calcd, 463.18039. found, 463.18092.

### Example 12

### 6-((4-(2-(3-Fluoro-6-methoxy-1,5-naphthyridin-4-yl)ethyl)bicyclo[2.2.1]heptan-1-ylamino)methyl)-2H-pyrido[3,2-b][1,4]oxazin-3(4H)-one

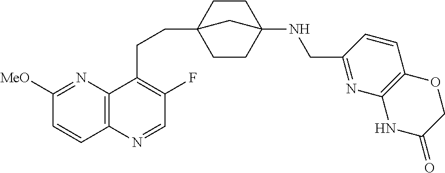 [SMILES: COc1ccc2ncc(F)c(CCC34CCC(NCc5ccc6c(n5)NC(=O)CO6)(CC3)C4)c2n1]

### Step 1

### tert-Butyl 4-(2-(3-Fluoro-6-methoxy-1,5-naphthyridin-4-yl)ethyl)bicyclo[2.2.1]heptan-1-ylcarbamate

The title compound tert-butyl 4-(2-(3-fluoro-6-methoxy-1,5-naphthyridin-4-yl)ethyl)bicyclo[2.2.1]heptan-1-ylcarbamate (77.8 mg) was prepared from G (100 mg) and L (108 mg) in the same manner as described for Step 1 of EXAMPLE 1.

¹H NMR (CDCl₃): δ 1.45 (s, 9H), 1.60-1.94 (m, 12H), 3.14-3.19 (m, 2H), 4.07 (s, 3H), 4.76 (br, 1H), 7.07 (d, J=8.6 Hz, 1H), 8.16 (d, J=9.2 Hz, 1H), 8.59 (s, 1H).

MS (ESI⁺) m/z: 416 (MH⁺).

HRMS (ESI⁺) for C₂₃H₃₁FN₃O₃ (MH⁺): calcd, 416.23494. found, 416.23449.

### Step 2

### 4-(2-(3-Fluoro-6-methoxy-1,5-naphthyridin-4-yl)ethyl)bicyclo[2.2.1]heptan-1-amine

The title compound 4-(2-(3-fluoro-6-methoxy-1,5-naphthyridin-4-yl)ethyl)bicyclo[2.2.1]heptan-1-amine (212 mg) was prepared from tert-butyl 4-(2-(3-fluoro-6-methoxy-1,5-naphthyridin-4-yl)ethyl)bicyclo[2.2.1]heptan-1-ylcarbamate (370 mg) in the same manner as described for Step 2 of EXAMPLE 1.

¹H NMR (CDCl₃): δ 1.36 (s, 2H), 1.57-1.74 (m, 8H), 1.80-1.89 (m, 2H), 3.14-3.19 (m, 2H), 4.07 (s, 3H), 7.06 (d, J=9.1 Hz, 1H), 8.16 (d, J=9.1 Hz, 1H), 8.59 (s, 1H).

MS (ESI⁺) m/z: 316 (MH⁺).

HRMS (ESI⁺) for C₁₈H₂₃FN₃O (MH⁺): calcd, 316.18251. found, 316.18280.

### Step 3

### 6-((4-(2-(3-Fluoro-6-methoxy-1,5-naphthyridin-4-yl)ethyl)bicyclo[2.2.1]heptan-1-ylamino)methyl)-2H-pyrido[3,2-b][1,4]oxazin-3(4H)-one

The title compound 6-((4-(2-(3-fluoro-6-methoxy-1,5-naphthyridin-4-yl)ethyl)bicyclo[2.2.1]heptan-1-ylamino)methyl)-2H-pyrido[3,2-b][1,4]oxazin-3(4H)-one (50.2 mg) was prepared from 4-(2-(3-fluoro-6-methoxy-1,5-naphthyridin-4-yl)ethyl)bicyclo[2.2.1]heptan-1-amine (100 mg) and I (59.3 mg) in the same manner as described for Step 3 of EXAMPLE 1.

¹H NMR (CDCl₃): δ 1.40 (s, 2H), 1.58-1.94 (m, 10H), 3.15-3.19 (m, 2H), 3.82 (s, 2H), 4.06 (s, 3H), 4.63 (s, 2H), 6.95 (d, J=8.6 Hz, 1H), 7.06 (d, J=9.2 Hz, 1H), 7.20 (d, J=7.9 Hz, 1H), 8.17 (d, J=9.2 Hz, 1H), 8.60 (s, 1H).

MS (ESI⁺) m/z: 478 (MH⁺).

HRMS (ESI⁺) for C₂₆H₂₉FN₅O₃ (MH⁺): calcd, 478.22544. found, 478.22577.

### Example 13

### 6-((4-(2-(3-Chloro-6-methoxy-1,5-naphthyridin-4-yl)-2-hydroxyethyl)bicyclo[2.2.2]octan-1-ylamino)methyl)-2H-pyrido[3,2-b][1,4]oxazin-3(4H)-one (Enantiomer A and Enantiomer B)

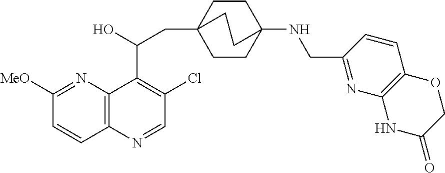 [SMILES: COc1ccc2ncc(Cl)c(C(O)CC34CCC(NCc5ccc6c(n5)NC(=O)CO6)(CC3)CC4)c2n1]

### Step 1

### tert-Butyl 4-(2-(3-Chloro-6-methoxy-1,5-naphthyridin-4-yl)-2-hydroxyethyl)bicyclo[2.2.2]octan-1-ylcarbamate (Enantiomer A and Enantiomer B)

To a solution of M (1.00 g) in tetrahydrofuran (37 mL) was added a solution of butyllithium (974 μL, 2.5 M in hexane) at −78° C., the mixture was stirred at the same temperature for 30 minutes. The mixture was added H (326 mg) at −78° C., the mixture was stirred at the same temperature for 4 hours. After quenching the reaction by adding 10% citric acid solution, the mixture was diluted with dichloromethane and washed with water. The organic extracts were washed with brine, dried over anhydrous sodium sulfate, filtered, and then concentrated in vacuo. Flash chromatography (silica, toluene:ethyl acetate=10:1) of the residue gave tert-butyl 4-(2-(3-chloro-6-methoxy-1,5-naphthyridin-4-yl)-2-hydroxyethyl)bicyclo[2.2.2]octan-1-ylcarbamate (303 mg). Optical resolution (CHIRALPAK IC, hexane:ethanol=25:75) of the racemate (303 mg) gave Enantiomer A (147 mg) and Enantiomer B (149 mg).

Enantiomer A: ¹H NMR (CDCl₃): δ 1.43 (s, 9H), 1.65-1.87 (m, 12H), 2.00 (dd, J=14.7, 11.0 Hz, 1H), 4.06 (s, 3H), 4.33 (br, 1H), 5.54 (dt, J=11.0, 1.8 Hz, 1H), 6.27 (d, J=11.0 Hz, 1H), 7.14 (d, J=8.6 Hz, 1H), 8.23 (d, J=9.2 Hz, 1H), 8.66 (s, 1H).

MS (ESI⁺) m/z: 462 (MH⁺).

HRMS (ESI⁺) for C₂₄H₃₃ClN₃O₄ (MH⁺): calcd, 462.21596. found, 462.21540.

Enantiomer B: ¹H NMR (CDCl₃): δ 1.43 (s, 9H), 1.65-1.87 (m, 12H), 2.00 (dd, J=14.7, 10.4 Hz, 1H), 4.06 (s, 3H), 4.33 (br, 1H), 5.54 (dt, J=11.0, 1.8 Hz, 1H), 6.27 (d, J=11.0 Hz, 1H), 7.14 (d, J=9.2 Hz, 1H), 8.23 (d, J=9.2 Hz, 1H), 8.66 (s, 1H).

MS (ESI⁺) m/z: 462 (MH⁺).

HRMS (ESI⁺) for C₂₄H₃₃ClN₃O₄ (MH⁺): calcd, 462.21596. found, 462.21540.

### Step 2

### 2-(4-Aminobicyclo[2.2.2]octan-1-yl)-1-(3-chloro-6-methoxy-1,5-naphthyridin-4-yl)ethanol (Enantiomer A)

The title compound 2-(4-aminobicyclo[2.2.2]octan-1-yl)-1-(3-chloro-6-methoxy-1,5-naphthyridin-4-yl)ethanol (75.5 mg) was prepared from tert-butyl 4-(2-(3-chloro-6-methoxy-1,5-naphthyridin-4-yl)-2-hydroxyethyl)bicyclo[2.2.2]octan-1-ylcarbamate (100 mg, Enantiomer A) in the same manner as described for Step 2 of EXAMPLE 1.

¹H NMR (CDCl₃): δ 1.25 (br, 2H), 1.41 (dd, J=14.7, 1.8 Hz, 1H), 1.59-1.83 (m, 12H), 2.01 (dd, J=14.7, 9.2 Hz, 1H), 4.06 (s, 3H), 5.45 (d, J=10.4 Hz, 1H), 6.30 (br, 1H), 7.15 (d, J=9.2 Hz, 1H), 8.23 (d, J=9.2 Hz, 1H), 8.66 (s, 1H).

MS (ESI⁺) m/z: 362 (MH⁺).

HRMS (ESI⁺) for C₁₉H₂₅ClN₃O₂ (MH⁺): calcd, 362.16353. found, 362.16285.

Enantiomer B of 2-(4-aminobicyclo[2.2.2]octan-1-yl)-1-(3-chloro-6-methoxy-1,5-naphthyridin-4-yl)ethanol (132 mg) was prepared in the same manner from tert-butyl 4-(2-(3-chloro-6-methoxy-1,5-naphthyridin-4-yl)-2-hydroxyethyl)bicyclo[2.2.2]octan-1-ylcarbamate (170 mg, Enantiomer B).

¹H NMR (DMSO-d₆): δ 1.14 (br, 2H), 1.32-1.65 (m, 13H), 2.3 (dd, J=14.7, 9.2 Hz, 1H), 4.03 (s, 3H), 5.45 (d, J=7.9 Hz, 1H), 5.78 (br, 1H), 7.31 (d, J=9.2 Hz, 1H), 8.31 (d, J=8.6 Hz, 1H), 8.72 (s, 1H).

MS (ESI⁺) m/z: 362 (MH⁺).

HRMS (ESI⁺) for C₁₉H₂₅ClN₃O₂ (MH⁺): calcd, 362.16353. found, 362.16416.

### Step 3

### 6-((4-(2-(3-Chloro-6-methoxy-1,5-naphthyridin-4-yl)-2-hydroxyethyl)bicyclo[2.2.2]octan-1-ylamino)methyl)-2H-pyrido[3,2-b][1,4]oxazin-3(4H)-one (Enantiomer A)

The title compound 6-((4-(2-(3-chloro-6-methoxy-1,5-naphthyridin-4-yl)-2-hydroxyethyl)bicyclo[2.2.2]octan-1-ylamino)methyl)-2H-pyrido[3,2-b][1,4]oxazin-3(4H)-one (77.8 mg) was prepared from 2-(4-aminobicyclo[2.2.2]octan-1-yl)-1-(3-chloro-6-methoxy-1,5-naphthyridin-4-yl)ethanol (60.0 mg, Enantiomer A) and I (31.1 mg) in the same manner as described for Step 3 of EXAMPLE 1.

¹H NMR (DMSO-d₆): δ 1.42-1.68 (m, 14H), 2.05 (dd, J=14.7, 9.2 Hz, 1H), 3.58 (s, 2H), 4.03 (s, 3H), 4.58 (s, 2H), 5.47 (d, J=7.3 Hz, 1H), 5.79 (br, 1H), 6.99 (d, J=7.9 Hz, 1H), 7.26 (d, J=7.9 Hz, 1H), 7.32 (d, J=9.2 Hz, 1H), 8.31 (d, J=9.2 Hz, 1H), 8.73 (s, 1H), 11.12 (br, 1H).

MS (ESI⁺) m/z: 524 (MH⁺).

HRMS (ESI⁺) for C₂₇H₃₁ClN₅O₄ (MH⁺): calcd, 524.20646. found, 524.20636.

Enantiomer B of 6-((4-(2-(3-chloro-6-methoxy-1,5-naphthyridin-4-yl)-2-hydroxyethyl)bicyclo[2.2.2]octan-1-ylamino)methyl)-2H-pyrido[3,2-b][1,4]oxazin-3(4H)-one (76.3 mg) was prepared in the same manner from 2-(4-aminobicyclo[2.2.2]octan-1-yl)-1-(3-chloro-6-methoxy-1,5-naphthyridin-4-yl)ethanol (60.0 mg, Enantiomer B).

¹H NMR (DMSO-d₆): δ 1.44-1.69 (m, 14H), 2.05 (dd, J=14.7, 9.2 Hz, 1H), 3.58 (s, 2H), 4.03 (s, 3H), 4.58 (s, 2H), 5.47 (d, J=7.3 Hz, 1H), 5.80 (br, 1H), 6.99 (d, J=7.9 Hz, 1H), 7.26 (d, J=7.9 Hz, 1H), 7.32 (d, J=9.2 Hz, 1H), 8.31 (d, J=9.2 Hz, 1H), 8.73 (s, 1H), 11.13 (br, 1H).

MS (ESI⁺) m/z: 524 (MH⁺).

HRMS (ESI⁺) for C₂₇H₃₁ClN₅O₄ (MH⁺): calcd, 524.20646. found, 524.20718.

### Example 14

### 6-((4-(2-(3-Chloro-6-methoxy-1,5-naphthyridin-4-yl)-1-hydroxyethyl)bicyclo[2.2.2]octan-1-ylamino)methyl)-2H-pyrido[3,2-b][1,4]oxazin-3(4H)-one Hydrochloride (Enantiomer A and Enantiomer B)

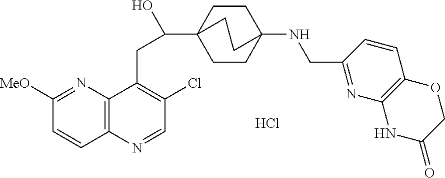 [SMILES: COc1ccc2ncc(Cl)c(CC(O)C34CCC(NCc5ccc6c(n5)NC(=O)CO6)(CC3)CC4)c2n1 ; Cl]

### Step 1

### tert-Butyl 4-(2-(3-Chloro-6-methoxy-1,5-naphthyridin-4-yl)-1-hydroxyethyl)bicyclo[2.2.2]octan-1-ylcarbamate (Enantiomer A and Enantiomer B)

To a solution of O (3.34 g) in tetrahydrofuran (160 mL) was added a solution of lithium diisopropyl amide (16.0 mL, 1.0 M in tetrahydrofuran) at −78° C., the mixture was stirred at the same temperature for 1.5 hours. The resulting mixture was added A (1.35 g) at −78° C., the mixture was stirred at the same temperature for 2 hours. After quenching the reaction by adding 10% citric acid solution, the mixture was extracted with dichloromethane. The organic extracts were washed with water and brine, dried over anhydrous sodium sulfate, filtered, and then concentrated in vacuo. Flash chromatography (silica, hexane:ethyl acetate=2:1) of the residue gave 4-(2-(3-chloro-6-methoxy-1,5-naphthyridin-4-yl)-1-hydroxyethyl)bicyclo[2.2.2]octan-1-ylcarbamate (1.57 g). Optical resolution (CHIRALPAK IC, hexane:ethanol=30:70) of the racemate (820 mg) gave Enantiomer A (401 mg) and Enantiomer B (414 mg).

Enantiomer A: ¹H NMR (CDCl₃): δ 1.44 (s, 9H), 1.65-1.96 (m, 12H), 3.35 (d, J=11.6 Hz, 1H), 3.43-3.56 (m, 2H), 3.67 (br, 1H), 4.07 (s, 3H), 4.37 (br, 1H), 7.11 (d, J=9.2 Hz, 1H), 8.20 (d, J=9.2 Hz, 1H), 8.71 (s, 1H).

MS (ESI⁺) m/z: 462 (MH⁺).

HRMS (ESI⁺) for C₂₄H₃₃ClN₃O₄ (MH⁺): calcd, 462.21596. found, 462.21571.

Enantiomer B: ¹H NMR (CDCl₃): δ 1.44 (s, 9H), 1.65-1.96 (m, 12H), 3.35 (d, J=12.8 Hz, 1H), 3.46 (t, J=10.4 Hz, 1H) 3.54 (dd, J=10.4, 3.7 Hz, 1H), 3.68 (br, 1H), 4.07 (s, 3H), 4.37 (br, 1H), 7.11 (d, J=9.2 Hz, 1H), 8.20 (d, J=9.2 Hz, 1H), 8.71 (s, 1H).

MS (ESI⁺) m/z: 462 (MH⁺).

HRMS (ESI⁺) for C₂₄H₃₃ClN₃O₄ (MH⁺): calcd, 462.21596. found, 462.21567.

### Step 2

### 1-(4-Aminobicyclo[2.2.2]octan-1-yl)-2-(3-chloro-6-methoxy-1,5-naphthyridin-4-yl)ethanol (Enantiomer A)

The title compound 1-(4-aminobicyclo[2.2.2]octan-1-yl)-2-(3-chloro-6-methoxy-1,5-naphthyridin-4-yl)ethanol (256 mg) was prepared from tert-butyl 4-(2-(3-chloro-6-methoxy-1,5-naphthyridin-4-yl)-1-hydroxyethyl)bicyclo[2.2.2]octan-1-ylcarbamate (340 mg, Enantiomer A) in the same manner as described for Step 2 of EXAMPLE 1.

¹H NMR (CDCl₃): δ 1.55-1.65 (m, 6H), 1.65-1.84 (m, 6H), 3.36 (dd, J=12.2, 1.8 Hz, 1H), 3.48 (t, J=12.2 Hz, 1H), 3.55 (d, J=11.6 Hz, 1H), 4.08 (s, 3H), 7.12 (d, J=9.2 Hz, 1H), 8.20 (d, J=9.2 Hz, 1H), 8.71 (s, 1H).

MS (ESI⁺) m/z: 362 (MH⁺).

HRMS (ESI⁺) for C₁₉H₂₅ClN₃O₂ (MH⁺): calcd, 362.16353. found, 362.16364.

Enantiomer B of 1-(4-aminobicyclo[2.2.2]octan-1-yl)-2-(3-chloro-6-methoxy-1,5-naphthyridin-4-yl)ethanol (46.6 mg) was prepared in the same manner from tert-butyl 4-(2-(3-chloro-6-methoxy-1,5-naphthyridin-4-yl)-1-hydroxyethyl)bicyclo[2.2.2]octan-1-ylcarbamate (64.4 mg, Enantiomer B).

¹H NMR (DMSO-d₆): δ 1.21 (s, 2H), 1.37-1.47 (m, 6H), 1.49-1.68 (m, 6H), 3.20-3.35 (m, 2H), 3.61-3.69 (m, 1H), 4.01 (s, 3H), 4.04 (d, J=6.1 Hz, 1H), 7.25 (d, J=9.2 Hz, 1H), 8.25 (d, J=9.2 Hz, 1H), 8.70 (s, 1H).

MS (ESI⁺) m/z: 362 (MH⁺).

HRMS (ESI⁺) for C₁₉H₂₅ClN₃O₂ (MH⁺): calcd, 362.16353. found, 362.16381.

### Step 3

### 6-((4-(2-(3-Chloro-6-methoxy-1,5-naphthyridin-4-yl)-1-hydroxyethyl)bicyclo[2.2.2]octan-1-ylamino)methyl)-2H-pyrido[3,2-b][1,4]oxazin-3(4H)-one (Enantiomer A)

The title compound 6-((4-(2-(3-chloro-6-methoxy-1,5-naphthyridin-4-yl)-1-hydroxyethyl)bicyclo[2.2.2]octan-1-ylamino)methyl)-2H-pyrido[3,2-b][1,4]oxazin-3(4H)-one (231 mg) was prepared from 1-(4-aminobicyclo[2.2.2]octan-1-yl)-2-(3-chloro-6-methoxy-1,5-naphthyridin-4-yl)ethanol (200 mg, Enantiomer A) and I (103 mg) in the same manner as described for Step 3 of EXAMPLE 1.

¹H NMR (DMSO-d₆): δ 1.45-1.73 (m, 12H), 3.21-3.36 (m, 2H), 3.60-3.74 (m, 2H), 4.01 (s, 3H), 4.11 (br, 1H), 4.60 (s, 2H), 7.04 (d, J=7.9 Hz, 1H), 7.25 (d, J=9.2 Hz, 1H), 7.30 (d, J=6.7 Hz, 1H), 8.25 (d, J=9.2 Hz, 1H), 8.71 (s, 1H), 11.17 (br, 1H).

MS (ESI⁺) m/z: 524 (MH⁺).

HRMS (ESI⁺) for C₂₇H₃₁ClN₅O₄ (MH⁺): calcd, 524.20646. found, 524.20656.

Enantiomer B of 6-((4-(2-(3-chloro-6-methoxy-1,5-naphthyridin-4-yl)-1-hydroxyethyl)bicyclo[2.2.2]octan-1-ylamino)methyl)-2H-pyrido[3,2-b][1,4]oxazin-3(4H)-one (45.5 mg) was prepared in the same manner from 1-(4-aminobicyclo[2.2.2]octan-1-yl)-2-(3-chloro-6-methoxy-1,5-naphthyridin-4-yl)ethanol (40.0 mg, Enantiomer B).

¹H NMR (DMSO-d₆): δ 1.45-1.90 (m, 12H), 3.21-3.36 (m, 2H), 3.58-3.71 (m, 2H), 4.01 (s, 3H), 4.10 (s, 1H), 4.60 (s, 2H), 7.02 (d, J=7.9 Hz, 1H), 7.25 (d, J=9.2 Hz, 1H), 8.25 (d, J=9.2 Hz, 1H), 8.71 (s, 1H), 11.15 (br, 1H).

MS (ESI⁺) m/z: 524 (MH⁺).

HRMS (ESI⁺) for C₂₇H₃₁ClN₅O₄ (MH⁺): calcd, 524.20646. found, 524.20621.

### Step 4

### 6-((4-(2-(3-Chloro-6-methoxy-1,5-naphthyridin-4-yl)-1-hydroxyethyl)bicyclo[2.2.2]octan-1-ylamino)methyl)-2H-pyrido[3,2-b][1,4]oxazin-3(4H)-one Hydrochloride (Enantiomer A)

The title compound 6-((4-(2-(3-chloro-6-methoxy-1,5-naphthyridin-4-yl)-1-hydroxyethyl)bicyclo[2.2.2]octan-1-ylamino)methyl)-2H-pyrido[3,2-b][1,4]oxazin-3(4H)-one hydrochloride (169 mg) was prepared from 6-((4-(2-(3-chloro-6-methoxy-1,5-naphthyridin-4-yl)-1-hydroxyethyl)bicyclo[2.2.2]octan-1-ylamino)methyl)-2H-pyrido[3,2-b][1,4]oxazin-3(4H)-one (150 mg, Enantiomer A) in the same manner as described for Step 4 of EXAMPLE 3.

¹H NMR (DMSO-d₆): δ 1.69-1.80 (m, 6H), 1.85-1.94 (m, 6H), 3.23 (t, J=11.6 Hz, 1H), 3.37 (dd, J=11.6, 2.4 Hz, 1H), 3.72 (dd, J=10.4, 2.4 Hz, 1H), 4.02 (s, 3H), 4.68 (s, 2H), 7.27 (d, J=9.2 Hz, 1H), 7.28 (d, J=8.6 Hz, 1H), 7.44 (d, J=7.9 Hz, 1H), 8.27 (d, J=8.6 Hz, 1H), 8.72 (s, 1H), 9.08 (br, 2H), 11.32 (br, 1H).

MS (ESI⁺) m/z: 524 (MH⁺) (as free base).

HRMS (ESI⁺) for C₂₇H₃₁ClN₅O₄ (MH⁺) (as free base): calcd, 524.20646. found, 524.20644.

Enantiomer B of 6-((4-(2-(3-chloro-6-methoxy-1,5-naphthyridin-4-yl)-1-hydroxyethyl)bicyclo[2.2.2]octan-1-ylamino)methyl)-2H-pyrido[3,2-b][1,4]oxazin-3(4H)-one hydrochloride (165 mg) was prepared in the same manner from 6-((4-(2-(3-chloro-6-methoxy-1,5-naphthyridin-4-yl)-1-hydroxyethyl)bicyclo[2.2.2]octan-1-ylamino)methyl)-2H-pyrido[3,2-b][1,4]oxazin-3(4H)-one (150 mg, Enantiomer B).

¹H NMR (DMSO-d₆): δ 1.69-1.74 (m, 6H), 1.85-1.89 (m, 6H), 3.23 (t, J=11.0 Hz, 1H), 3.37 (dd, J=12.2, 2.4 Hz, 1H), 3.72 (dd, J=11.0, 2.4 Hz, 1H), 4.02 (s, 3H), 4.68 (s, 2H), 7.25 (d, J=8.6 Hz, 1H), 7.27 (d, J=8.6 Hz, 1H), 7.45 (d, J=7.9 Hz, 1H), 8.27 (d, J=8.6 Hz, 1H), 8.72 (s, 1H), 9.00 (br, 2H), 11.32 (br, 1H).

MS (ESI⁺) m/z: 524 (MH⁺) (as free base).

HRMS (ESI⁺) for C₂₇H₃₁ClN₅O₄ (MH⁺) (as free base): calcd, 524.20646. found, 524.20611.

### Example 15

### 6-((4-((3-Chloro-6-methoxy-1,5-naphthyridin-4-yloxy)methyl)bicyclo[2.2.2]octan-1-ylamino)methyl)-2H-pyrido[3,2-b][1,4]oxazin-3(4H)-one

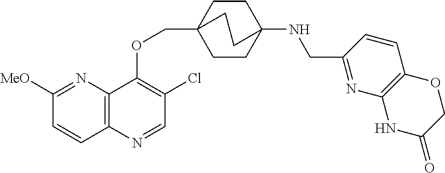 [SMILES: COc1ccc2ncc(Cl)c(OCC34CCC(NCc5ccc6c(n5)NC(=O)CO6)(CC3)CC4)c2n1]

### Step 1

### Methyl 4-((3-Chloro-6-methoxy-1,5-naphthyridin-4-yloxy)methyl)bicyclo[2.2.2]octane-1-carboxylate

To a solution of M.3 (1.82 g) in N,N-dimethylformamide (86 mL) was added sodium hydride (436 mg, 50% in mineral oil) and P (3.00 g) under cooling with ice, the mixture was stirred at the room temperature for 6 hours, and then concentrated in vacuo. After dilution of the residue with dichloromethane, the mixture was washed with water, 10% hydrochloric acid and brine. The organic extracts were dried over anhydrous sodium sulfate, filtered, and then concentrated in vacuo. Flash chromatography (silica, hexane:ethyl acetate=5:1) of the residue gave methyl 4-((3-chloro-6-methoxy-1,5-naphthyridin-4-yloxy)methyl)bicyclo[2.2.2]octane-1-carboxylate (1.51 g).

¹H NMR (CDCl₃): δ 1.65-1.76 (m, 6H), 1.82-1.92 (m, 6H), 3.67 (s, 3H), 4.05 (s, 3H), 4.40 (s, 2H), 7.09 (d, J=9.2 Hz, 1H), 8.15 (d, J=9.2 Hz, 1H), 8.64 (s, 1H).

MS (ESI⁺) m/z: 391 (MH⁺).

HRMS (ESI⁺) for C₂₀H₂₄ClN₂O₄ (MH⁺): calcd, 391.24109. found, 391.24095.

### Step 2

### 4-((3-Chloro-6-methoxy-1,5-naphthyridin-4-yloxy)methyl)bicyclo[2.2.2]octane-1-carboxylic Acid

To a solution of methyl 4-((3-chloro-6-methoxy-1,5-naphthyridin-4-yloxy)methyl)bicyclo[2.2.2]octane-1-carboxylate (1.40 g) in methanol (28.6 mL) was added 1 N sodium hydroxide solution (14.3 mL), the mixture was stirred at 70° C. for 3 hours, and then concentrated in vacuo. After dilution of the residue with water and 10% hydrochloric acid, the resulting precipitates were collected by filtration and washed with water to give 4-((3-chloro-6-methoxy-1,5-naphthyridin-4-yloxy)methyl)bicyclo[2.2.2]octane-1-carboxylic acid (1.30 g).

¹H NMR (CDCl₃): δ 1.71-1.75 (m, 6H), 1.89-1.93 (m, 6H), 4.05 (s, 3H), 4.42 (s, 2H), 7.10 (d, J=9.2 Hz, 1H), 8.17 (d, J=9.2 Hz, 1H), 8.65 (s, 1H).

MS (ESI⁺) m/z: 377 (MH⁺).

HRMS (ESI⁺) for C₁₉H₂₂ClN₂O₄ (MH⁺): calcd, 377.12681. found, 377.12754.

### Step 3

### 4-((3-Chloro-6-methoxy-1,5-naphthyridin-4-yloxy)methyl)bicyclo[2.2.2]octan-1-amine

A mixture of 4-((3-chloro-6-methoxy-1,5-naphthyridin-4-yloxy)methyl)bicyclo[2.2.2]octane-1-carboxylic acid (1.20 g), triethylamine (488 μL) and diphenyl phosphoryl azide (755 μL) in toluene (32 mL) was stirred at room temperature for 2 hours, reflux at 120° C. and concentrated in vacuo. A solution of the residue in 1,4-dioxane (16 mL) and 6 N hydrochloric acid (16 mL) was stirred at room temperature for 30 minutes and then concentrated in vacuo. After dilution of the residue with dichloromethane, the mixture was washed with 1 N sodium hydroxide solution and brine, dried over anhydrous sodium sulfate, filtered, and then concentrated in vacuo. Treatment of the residue with hexane gave 4-((3-chloro-6-methoxy-1,5-naphthyridin-4-yloxy)methyl)bicyclo[2.2.2]octan-1-amine (788 mg).

¹H NMR (CDCl₃): δ 1.57-1.61 (m, 6H), 1.70-1.78 (m, 6H), 4.05 (s, 3H), 4.40 (s, 2H), 7.09 (d, J=9.2 Hz, 1H), 8.15 (d, J=9.2 Hz, 1H), 8.64 (s, 1H).

MS (ESI⁺) m/z: 348 (MH⁺).

HRMS (ESI⁺) for C₁₈H₂₃ClN₃O₂ (MH⁺): calcd, 348.14788. found, 348.14755.

### Step 4

### 6-((4-((3-Chloro-6-methoxy-1,5-naphthyridin-4-yloxy)methyl)bicyclo[2.2.2]octan-1-ylamino)methyl)-2H-pyrido[3,2-b][1,4]oxazin-3(4H)-one

The title compound 6-((4-((3-chloro-6-methoxy-1,5-naphthyridin-4-yloxy)methyl)bicyclo[2.2.2]octan-1-ylamino)methyl)-2H-pyrido[3,2-b][1,4]oxazin-3(4H)-one (92.0 mg) was prepared from 4-((3-chloro-6-methoxy-1,5-naphthyridin-4-yloxy)methyl)bicyclo[2.2.2]octan-1-amine (100 mg) and I (51.2 mg) in the same manner as described for Step 3 of EXAMPLE 1.

¹H NMR (CDCl₃): δ 1.65-1.86 (m, 12H), 3.76 (s, 2H), 4.05 (s, 3H), 4.41 (s, 2H), 4.62 (s, 2H), 6.95 (d, J=7.9 Hz, 1H), 7.09 (d, J=9.2 Hz, 1H), 7.19 (d, J=8.6 Hz, 1H), 8.16 (d, J=9.2 Hz, 1H), 8.64 (s, 1H).

MS (ESI⁺) m/z: 510 (MH⁺).

HRMS (ESI⁺) for C₂₆H₂₉ClN₅O₄ (MH⁺): calcd, 510.19081. found, 510.19066.

### Example 16

### 6-((1-(2-(3-Chloro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one Hydrochloride

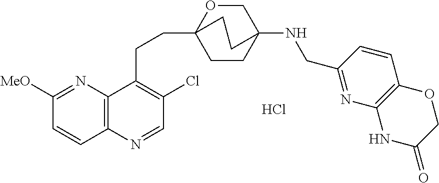 [SMILES: COc1ccc2ncc(Cl)c(CCC34CCC(NCc5ccc6c(n5)NC(=O)CO6)(CC3)CO4)c2n1 ; Cl]

### Step 1

### tert-Butyl 1-(2-(3-Chloro-6-methoxy-1,5-naphthyridin-4-yl)ethyl)-2-oxabicyclo[2.2.2]octan-4-ylcarbamate

The title compound tert-butyl 1-(2-(3-chloro-6-methoxy-1,5-naphthyridin-4-yl)ethyl)-2-oxabicyclo[2.2.2]octan-4-ylcarbamate (2.40 mg) was prepared from Q (30.2 mg) and M (22.8 mg) in the same manner as described for Step 1 of EXAMPLE 1

¹H NMR (CDCl₃): δ 1.44 (s, 9H), 1.67-1.75 (m, 2H), 1.78-1.93 (m, 4H), 1.98-2.20 (m, 4H), 3.29-3.34 (m, 2H), 3.98 (s, 2H), 4.07 (s, 3H), 4.30 (br, 1H), 7.09 (d, J=9.1 Hz, 1H), 8.14 (d, J=9.1 Hz, 1H), 8.64 (s, 1H).

MS (ESI⁺) m/z: 448 (MH⁺).

HRMS (ESI⁺) for C₂₃H₃₁ClN₃O₄ (MH⁺): calcd, 448.20031. found, 448.20024.

### Step 2

### 1-(2-(3-Chloro-6-methoxy-1,5-naphthyridin-4-yl)ethyl)-2-oxabicyclo[2.2.2]octan-4-amine

The title compound 1-(2-(3-chloro-6-methoxy-1,5-naphthyridin-4-yl)ethyl)-2-oxabicyclo[2.2.2]octan-4-amine (35.1 mg) was prepared from tert-butyl 1-(2-(3-chloro-6-methoxy-1,5-naphthyridin-4-yl)ethyl)-2-oxabicyclo[2.2.2]octan-4-ylcarbamate (45.0 mg) in the same manner as described for Step 2 of EXAMPLE 1.

¹H NMR (DMSO-d₆): δ 1.29 (s, 2H), 1.47-1.74 (m, 8H), 1.78-1.88 (m, 2H), 3.05-3.13 (m, 2H), 3.44 (s, 2H), 4.02 (s, 3H), 7.22 (d, J=9.2 Hz, 1H), 8.26 (d, J=9.2 Hz, 1H), 8.74 (s, 1H).

MS (CI⁺) m/z: 348 (MH⁺).

HRMS (CI⁺) for C₁₈H₂₃ClN₃O₂ (MH⁺): calcd, 348.1479. found, 348.1477.

### Step 3

### 6-((1-(2-(3-Chloro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one

The title compound 6-((1-(2-(3-chloro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one (25.1 mg) was prepared from 1-(2-(3-chloro-6-methoxy-1,5-naphthyridin-4-yl)ethyl)-2-oxabicyclo[2.2.2]octan-4-amine (30.0 mg) and I (16.1 mg) in the same manner as described for Step 3 of EXAMPLE 1.

¹H NMR (DMSO-d₆): δ 1.54-1.80 (m, 8H), 1.82-1.93 (m, 3H), 3.23-3.27 (m, 2H), 3.59 (s, 2H), 3.63 (d, J=6.7 Hz, 2H), 4.03 (s, 3H), 4.59 (s, 2H), 7.01 (d, J=7.9 Hz, 1H), 7.27 (d, J=9.1 Hz, 1H), 7.28 (d, J=7.9 Hz, 1H), 8.26 (d, J=9.1 Hz, 1H), 8.72 (s, 1H), 11.15 (br, 1H).

MS (ESI⁺) m/z: 510 (MH⁺).

HRMS (ESI⁺) for C₂₆H₂₉ClN₅O₄ (MH⁺): calcd, 510.19081. found, 510.19054.

### Step 4

### 6-((1-(2-(3-Chloro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one Hydrochloride

The title compound 6-((1-(2-(3-chloro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one hydrochloride (41.1 mg) was prepared from 6-((1-(2-(3-chloro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one (39.1 mg) in the same manner as described for Step 4 of EXAMPLE 3.

¹H NMR (DMSO-d₆): δ 1.61-1.69 (m, 2H), 1.82-1.93 (m, 2H), 1.95-2.05 (m, 6H), 3.22-3.30 (m, 2H), 3.93 (s, 2H), 4.04 (s, 3H), 4.07-4.15 (m, 2H), 4.69 (s, 2H), 7.22 (d, J=8.6 Hz, 1H), 7.29 (d, J=8.6 Hz, 1H), 7.46 (d, J=8.6 Hz, 1H), 8.28 (d, J=8.6 Hz, 1H), 8.74 (s, 1H), 9.29 (s, 2H), 11.33 (s, 1H).

MS (ESI⁺) m/z: 510 (MH⁺) (as free base).

HRMS (ESI⁺) for C₂₆H₂₉ClN₅O₄ (MH⁺) (as free base): calcd, 510.19081. found, 510.19133.

### Example 17

### 6-((1-(2-(6-Methoxy-1,5-naphthyridin-4-yl)ethyl)-2-oxabicyclo[2.2.2]octan-4-ylamino)methyl)-2H-pyrido[3,2-b][1,4]oxazin-3(4H)-one Hydrochloride

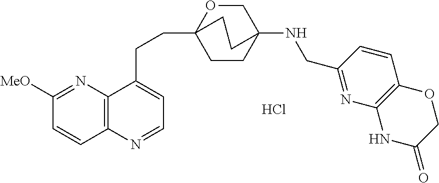 [SMILES: COc1ccc2nccc(CCC34CCC(NCc5ccc6c(n5)NC(=O)CO6)(CC3)CO4)c2n1 ; Cl]

### Step 1

### tert-Butyl 1-(2-(6-Methoxy-1,5-naphthyridin-4-yl)ethyl)-2-oxabicyclo[2.2.2]octan-4-ylcarbamate

To a solution of B (200 mg) in tetrahydrofuran (3.4 mL) was added a solution of 9-borabicyclo(3.3.1)nonane dimer (3.2 mL, 0.5 M in tetrahydrofuran) under cooling with ice, the mixture was stirred at room temperature for 4 hours. The reaction was quenched by adding water. 8-Bromo-2-methoxy-1,5-naphthyridine (189 mg), tetrakis(triphenylphosphine)palladium (182 mg), potassium phosphate (1.18 g) and ethanol/water (1.85 mL, 4:1) were added to the mixture. The resulting mixture was stirred at 70° C. for overnight and then concentrated in vacuo. After dilution of the residue with ethyl acetate, the mixture was washed with water and brine, dried over anhydrous sodium sulfate, filtered, and then concentrated in vacuo. Flash chromatography (silica, hexane:ethyl acetate=1:1) of the residue gave tert-butyl 1-(2-(6-methoxy-1,5-naphthyridin-4-yl)ethyl)-2-oxabicyclo[2.2.2]octan-4-ylcarbamate (139 mg).

¹H NMR (CDCl₃): δ 1.44 (s, 9H), 1.71-1.90 (m, 6H), 1.92-2.18 (m, 4H), 3.13-3.21 (m, 2H), 3.99 (s, 2H), 4.07 (s, 3H), 4.30 (br, 1H), 7.10 (d, J=9.2 Hz, 1H), 7.38 (d, J=4.3 Hz, 1H), 8.17 (d, J=9.2 Hz, 1H), 8.64 (d, J=4.3 Hz, 1H).

MS (ESI⁺) m/z: 414 (MH⁺).

HRMS (ESI⁺) for C₂₃H₃₂N₃O₄ (MH⁺): calcd, 414.23928. found, 414.24013.

### Step 2

### 1-(2-(6-Methoxy-1,5-naphthyridin-4-yl)ethyl)-2-oxabicyclo[2.2.2]octan-4-amine

The title compound 1-(2-(6-methoxy-1,5-naphthyridin-4-yl)ethyl)-2-oxabicyclo[2.2.2]octan-4-amine (71.2 mg) was prepared from tert-butyl 1-(2-(6-methoxy-1,5-naphthyridin-4-yl)ethyl)-2-oxabicyclo[2.2.2]octan-4-ylcarbamate (95.0 mg) in the same manner as described for Step 2 of EXAMPLE 1.

¹H NMR (CDCl₃): δ 1.63-1.88 (m, 8H), 1.95-2.06 (m, 2H), 3.14-3.21 (m, 2H), 3.67 (s, 2H), 4.07 (s, 3H), 7.10 (d, J=9.2 Hz, 1H), 7.38 (d, J=4.9 Hz, 1H), 8.18 (d, J=9.2 Hz, 1H), 8.64 (d, J=4.3 Hz, 1H).

MS (ESI⁺) m/z: 314 (MH⁺).

HRMS (ESI⁺) for C₁₈H₂₄N₃O₂ (MH⁺): calcd, 314.18685. found, 314.18768.

### Step 3

### 6-((1-(2-(6-M ethoxy-1,5-naphthyridin-4-yl)ethyl)-2-oxabicyclo[2.2.2]octan-4-ylamino)methyl)-2H-pyrido[3,2-b][1,4]oxazin-3(4H)-one

The title compound 6-((1-(2-(6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one (39.6 mg) was prepared from 1-(2-(6-methoxy-1,5-naphthyri din-4-yl)ethyl)-2-oxabicyclo[2.2.2]octan-4-amine (50.0 mg) and I (29.8 mg) in the same manner as described for Step 3 of EXAMPLE 1.

¹H NMR (DMSO-d₆): δ 1.60-1.78 (m, 8H), 1.79-1.88 (m, 3H), 3.06-3.11 (m, 2H), 3.59 (s, 2H), 3.63 (d, J=6.1 Hz, 2H), 4.01 (s, 3H), 4.59 (s, 2H), 7.01 (d, J=7.9 Hz, 1H), 7.23 (d, J=9.2 Hz, 1H), 7.27 (d, J=8.6 Hz, 1H), 7.51 (d, J=4.9 Hz, 1H), 8.22 (d, J=8.6 Hz, 1H), 8.64 (d, J=4.3 Hz, 1H), 11.15 (br, 1H).

MS (ESI⁺) m/z: 476 (MH⁺).

HRMS (ESI⁺) for C₂₆H₃₀N₅O₄ (MH⁺): calcd, 476.22978. found, 476.22907.

### Step 4

### 6((1-(2-(6-Methoxy-1,5-naphthyridin-4-yl)ethyl)-2-oxabicyclo[2.2.2]octan-4-ylamino)methyl)-2H-pyrido[3,2-b][1,4]oxazin-3(4H)-one Hydrochloride

The title compound 6-((1-(2-(6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one hydrochloride (42.0 mg) was prepared from 6-((1-(2-(6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one (42.5 mg) in the same manner as described for Step 4 of EXAMPLE 3.

¹H NMR (DMSO-d₆): δ 1.75-1.92 (m, 4H), 1.96-2.10 (m, 6H), 3.11-3.21 (m, 2H), 3.93 (s, 2H), 4.05 (s, 3H), 4.05 (d, J=6.1 Hz, 2H), 4.69 (s, 2H), 7.25 (d, J=8.6 Hz, 1H), 7.36 (d, J=9.2 Hz, 1H), 7.45 (d, J=7.9 Hz, 1H), 7.70 (d, J=4.9 Hz, 1H), 8.34 (d, J=9.2 Hz, 1H), 8.77 (d, J=4.9 Hz, 1H), 9.37 (s, 2H), 11.32 (s, 1H).

MS (ESI⁺) m/z: 476 (MH⁺) (as free base).

HRMS (ESI⁺) for C₂₆H₃₀N₅O₄ (MH⁺) (as free base): calcd, 476.22978. found, 476.22914.

### Example 18

### 6-((1-(2-(3-Fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one Hydrochloride

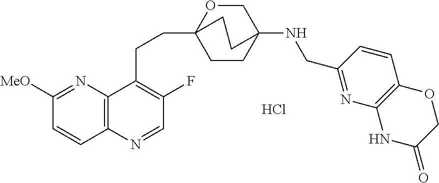 [SMILES: COc1ccc2ncc(F)c(CCC34CCC(NCc5ccc6c(n5)NC(=O)CO6)(CC3)CO4)c2n1 ; Cl]

### Step 1

### (E)-tert-Butyl 1-(2-(3-Fluoro-6-methoxy-1,5-naphthyridin-4-yl)vinyl)-2-oxabicyclo[2.2.2]octan-4-ylcarbamate

A suspension of B (0.99 g), L (1.00 g), palladium(II) acetate (87.3 mg) and silver carbonate (644 mg) in benzene (23 mL) was stirred under reflux for overnight. After dilution of the mixture with ethyl acetate, the insoluble materials were filtered off. The filtrate was washed with water and brine, dried over anhydrous sodium sulfate, filtered, and then concentrated in vacuo. Flash chromatography (silica, toluene:ethyl acetate=6:1) of the residue gave (E)-tert-butyl 1-(2-(3-fluoro-6-methoxy-1,5-naphthyridin-4-yl)vinyl)-2-oxabicyclo[2.2.2]octan-4-ylcarbamate (1.14 g).

¹H NMR (CDCl₃): δ 1.44 (s, 9H), 1.89-2.02 (m, 4H), 2.08-2.25 (m, 4H), 4.10 (s, 5H), 4.34 (brs, 1H), 7.07 (d, J=8.6 Hz, 1H), 7.20 (d, J=16.5 Hz, 1H), 7.38 (d, J=16.5 Hz, 1H), 8.16 (d, J=9.2 Hz, 1H), 8.62 (d, J=2.4 Hz, 1H).

MS (ESI⁺) m/z: 430 (MH⁺).

HRMS (ESI⁺) for C₂₃H₂₉FN₃O₄ (MH⁺): calcd, 430.21412. found, 430.21432.

### Step 2

### tert-Butyl 1-(2-(3-Fluoro-6-methoxy-1,5-naphthyridin-4-yl)ethyl)-2-oxabicyclo[2.2.2]octan-4-ylcarbamate

A suspension of (E)-tert-butyl 1-(2-(3-fluoro-6-methoxy-1,5-naphthyridin-4-yl)vinyl)-2-oxabicyclo[2.2.2]octan-4-ylcarbamate (13.4 g), 10% Pd—C (2.01 g) in N,N-dimethylformamide (156 mL) was stirred at room temperature for 1 hour under H₂ atmosphere (1 kg/cm²). After the insoluble materials were filtered off, the filtrate was concentrated in vacuo. Flash chromatography (silica, hexane:ether=3:1) of the residue gave tert-butyl 1-(2-(3-fluoro-6-methoxy-1,5-naphthyridin-4-yl)ethyl)-2-oxabicyclo[2.2.2]octan-4-ylcarbamate (12.4 g).

¹H NMR (CDCl₃): δ 1.43 (s, 9H), 1.71-1.93 (m, 6H), 1.91-2.07 (m, 4H), 3.15-3.23 (m, 2H), 3.96 (s, 2H), 4.08 (s, 3H), 4.29 (br, 1H), 7.05 (d, J=9.2 Hz, 1H), 8.16 (d, J=9.2 Hz, 1H), 8.59 (s, 1H).

MS (ESI⁺) m/z: 432 (MH⁺).

HRMS (ESI⁺) for C₂₃H₃₁FN₃O₄ (MH⁺): calcd, 432.22986. found, 432.23055.

### Step 3

### 1-(2-(3-Fluoro-6-methoxy-1,5-naphthyridin-4-yl)ethyl)-2-oxabicyclo[2.2.2]octan-4-amine

The title compound 1-(2-(3-fluoro-6-methoxy-1,5-naphthyridin-4-yl)ethyl)-2-oxabicyclo[2.2.2]octan-4-amine (1.14 g) was prepared from tert-butyl 1-(2-(3-fluoro-6-methoxy-1,5-naphthyridin-4-yl)ethyl)-2-oxabicyclo[2.2.2]octan-4-ylcarbamate (1.50 g) in the same manner as described for Step 2 of EXAMPLE 1.

¹H NMR (CDCl₃): δ 1.63-1.78 (m, 8H), 1.82-1.89 (m, 2H), 3.08-3.18 (m, 2H), 3.58 (s, 2H), 4.03 (s, 3H), 5.24 (br, 2H), 7.22 (d, J=9.2 Hz, 1H), 8.26 (d, J=9.2 Hz, 1H), 8.74 (s, 1H).

MS (ESI⁺) m/z: 332 (MH⁺).

HRMS (ESI⁺) for C₁₈H₂₃FN₃O₂ (MH⁺): calcd, 332.17743. found, 332.17750.

### Step 4

### 6-((1-(2-(3-Fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one

The title compound 6-((1-(2-(3-fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one (42.8 mg) was prepared from 1-(2-(3-fluoro-6-methoxy-1,5-naphthyridin-4-yl)ethyl)-2-oxabicyclo[2.2.2]octan-4-amine (40.0 mg) and I (22.6 mg) in the same manner as described for Step 3 of EXAMPLE 1.

¹H NMR (DMSO-d₆): δ 1.62-1.77 (m, 8H), 1.83-1.92 (m, 3H), 3.08-3.15 (m, 2H), 3.58 (s, 2H), 3.62 (d, J=6.1 Hz, 2H), 4.03 (s, 3H), 4.59 (s, 2H), 7.01 (d, J=7.9 Hz, 1H), 7.22 (d, J=9.2 Hz, 1H), 7.27 (d, J=7.9 Hz, 1H), 8.26 (d, J=9.2 Hz, 1H), 8.74 (s, 1H), 11.14 (br, 1H).

MS (ESI⁺) m/z: 494 (MH⁺).

HRMS (ESI⁺) for C₂₆H₂₉FN₅O₄ (MH⁺): calcd, 494.22036. found, 494.22013.

### Step 5

### 6-((1-(2-(3-Fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one Hydrochloride

The title compound 6-((1-(2-(3-fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one hydrochloride (40.0 mg) was prepared from 6-((1-(2-(3-fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one (40.0 mg) in the same manner as described for Step 4 of EXAMPLE 3.

¹H NMR (DMSO-d₆): δ 1.66-1.73 (m, 2H), 1.79-1.91 (m, 2H), 1.93-2.10 (m, 6H), 3.09-3.18 (m, 2H), 3.91 (s, 2H), 4.04 (s, 3H), 4.10 (t, J=6.1 Hz, 2H), 4.69 (s, 2H), 7.22 (d, J=7.9 Hz, 1H), 7.24 (d, J=8.6 Hz, 1H), 7.45 (d, J=7.9 Hz, 1H), 8.27 (d, J=8.6 Hz, 1H), 8.76 (s, 1H), 9.25-9.36 (s, 2H), 11.32 (s, 1H).

MS (ESI⁺) m/z: 494 (MH⁺) (as free base).

HRMS (ESI⁺) for C₂₆H₂₉FN₅O₄ (MH⁺) (as free base): calcd, 494.22036. found, 494.22017.

### Example 19

### (E)-6-((1-(2-(3-Fluoro-6-methoxy-1,5-naphthyridin-4-yl)vinyl)-2-oxabicyclo[2.2.2]octan-4-ylamino)methyl)-2H-pyrido[3,2-b][1,4]oxazin-3(4H)-one

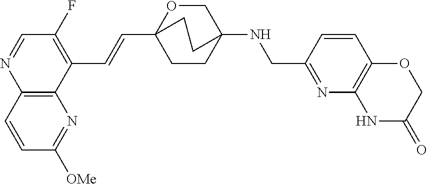 [SMILES: COc1ccc2ncc(F)c(/C=C/C34CCC(NCc5ccc6c(n5)NC(=O)CO6)(CC3)CO4)c2n1]

### Step 1

### (E)-1-(2-(3-Fluoro-6-methoxy-1,5-naphthyridin-4-yl)vinyl)-2-oxabicyclo[2.2.2]octan-4-amine

The title compound (E)-1-(2-(3-fluoro-6-methoxy-1,5-naphthyridin-4-yl)vinyl)-2-oxabicyclo[2.2.2]octan-4-amine (71.2 mg) was prepared from (E)-tert-butyl 1-(2-(3-fluoro-6-methoxy-1,5-naphthyridin-4-yl)vinyl)-2-oxabicyclo[2.2.2]octan-4-ylcarbamate (100 mg, see Step 1 of Example 18) in the same manner as described for Step 2 of EXAMPLE 1.

¹H NMR (CDCl₃): δ 1.35 (brs, 2H), 1.69-1.85 (m, 4H), 1.93-2.01 (m, 2H), 2.07-2.17 (m, 2H), 3.77 (s, 2H), 4.10 (s, 3H), 7.07 (d, J=9.1 Hz, 1H), 7.24 (d, J=16.3 Hz, 1H), 7.36 (d, J=17.0 Hz, 1H), 8.16 (d, J=9.1 Hz, 1H), 8.62 (d, J=2.4 Hz, 1H).

MS (ESI⁺) m/z: 330 (MH⁺).

HRMS (ESI⁺) for C₁₈H₂₁FN₃O₂ (MH⁺): calcd, 330.16178. found, 330.16207.

### Step 2

### (E)-6-((1-(2-(3-Fluoro-6-methoxy-1,5-naphthyridin-4-yl)vinyl)-2-oxabicyclo[2.2.2]octan-4-ylamino)methyl)-2H-pyrido[3,2-b][1,4]oxazin-3(4H)-one

The title compound (E)-6-((1-(2-(3-fluoro-6-methoxy-1,5-naphthyridin-4-yl)vinyl)-2-oxabicyclo[2.2.2]octan-4-ylamino)methyl)-2H-pyrido[3,2-b][1,4]oxazin-3(4H)-one (47.0 mg) was prepared from (E)-1-(2-(3-fluoro-6-methoxy-1,5-naphthyridin-4-yl)vinyl)-2-oxabicyclo[2.2.2]octan-4-amine (65.0 mg) and I (36.9 mg) in the same manner as described for Step 3 of EXAMPLE 1.

¹H NMR (CDCl₃): δ 1.65-1.81 (m, 4H), 1.84-2.00 (m, 5H), 3.66 (d, J=6.1 Hz, 2H), 3.72 (s, 2H), 4.02 (s, 3H), 4.59 (s, 2H), 7.03 (d, J=7.9 Hz, 1H), 7.17 (d, J=16.5 Hz, 1H), 7.26 (d, J=8.6 Hz, 1H), 7.27 (d, J=16.5 Hz, 1H), 7.28 (d, J=7.9 Hz, 1H), 8.27 (d, J=8.6 Hz, 1H), 8.79 (d, J=1.8 Hz, 1H), 11.16 (brs, 1H).

MS (ESI⁺) m/z: 492 (MH⁺).

HRMS (ESI⁺) for C₂₆H₂₇FN₅O₄ (MH⁺): calcd, 492.20471. found, 492.20511.

### Example 20

### 6-((1-(2-(3-Fluoro-6-methoxy-1,5-naphthyridin-4-yl)-1-hydroxyethyl)-2-oxabicyclo[2.2.2]octan-4-ylamino)methyl)-2H-pyrido[3,2-b][1,4]oxazin-3(4H)-one Hydrochloride (Enantiomer A and Enantiomer B)

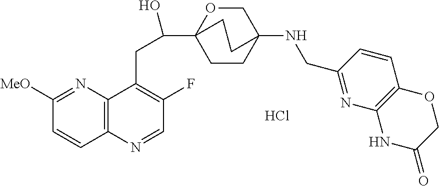 [SMILES: COc1ccc2ncc(F)c(CC(O)C34CCC(NCc5ccc6c(n5)NC(=O)CO6)(CC3)CO4)c2n1 ; Cl]

### Step 1

### tert-Butyl 1-(2-(3-Fluoro-6-methoxy-1,5-naphthyridin-4-yl)-1-hydroxyethyl)-2-oxabicyclo[2.2.2]octan-4-ylcarbamate (Enantiomer A and Enantiomer B)

To a suspension of R (1.13 g) in tetrahydrofuran (58.8 mL) was added a solution of lithium diisopropyl amide (5.88 mL, 1.0 M in tetrahydrofuran) at −78° C., the mixture was stirred at the same temperature for 50 minutes. F (500 mg) was added to the mixture at −78° C., the resulting mixture was stirred at the same temperature for 1.5 hours. After quenching the reaction by adding 10% citric acid solution, the mixture was extracted with dichloromethane. The organic extracts were washed with water and brine, dried over anhydrous sodium sulfate, filtered, and then concentrated in vacuo. Flash chromatography (silica, hexane:ethyl acetate=1:1) of the residue gave tert-butyl 1-(2-(3-fluoro-6-methoxy-1,5-naphthyridin-4-yl)-1-hydroxyethyl)-2-oxabicyclo[2.2.2]octan-4-ylcarbamate (446 mg). Optical resolution (CHIRALPAK IA, hexane:isopropanol:methyl tert-butyl ether=20:50:30) of the racemate (400 mg) gave Enantiomer A (206 mg) and Enantiomer B (197 mg).

Enantiomer A: ¹H NMR (DMSO-d₆): δ 1.38 (s, 9H), 1.71-2.08 (m, 8H), 2.99 (dd, J=12.6, 10.1 Hz, 1H), 3.30-3.36 (m, 1H), 3.70-3.77 (m, 3H), 4.02 (s, 3H), 4.48 (d, J=6.1 Hz, 2H), 6.59 (br, 1H), 7.20 (d, J=9.2 Hz, 1H), 8.25 (d, J=9.2 Hz, 1H), 8.72 (s, 1H).

MS (ESI⁺) m/z: 448 (MH⁺).

HRMS (ESI⁺) for C₂₃H₃₁FN₃O₅ (MH⁺): calcd, 448.22477. found, 448.22493.

Enantiomer B: ¹H NMR (DMSO-d₆): δ 1.36 (s, 9H), 1.72-2.01 (m, 8H), 2.99 (dd, J=12.1, 10.3 Hz, 1H), 3.28-3.36 (m, 1H), 3.70-3.80 (m, 3H), 4.02 (s, 3H), 4.48 (d, J=5.5 Hz, 2H), 6.59 (br, 1H), 7.20 (d, J=9.1 Hz, 1H), 8.25 (d, J=9.1 Hz, 1H), 8.72 (s, 1H).

MS (ESI⁺) m/z: 448 (MH⁺).

HRMS (ESI⁺) for C₂₃H₃₁FN₃O₅ (MH⁺): calcd, 448.22477. found, 448.22475.

### Step 2

### 1-(4-Amino-2-oxabicyclo[2.2.2]octan-1-yl)-2-(3-fluoro-6-methoxy-1,5-naphthyridin-4-yl)ethanol (Enantiomer A)

The title compound 1-(4-amino-2-oxabicyclo[2.2.2]octan-1-yl)-2-(3-fluoro-6-methoxy-1,5-naphthyridin-4-yl)ethanol (122 mg) was prepared from tert-butyl 1-(2-(3-fluoro-6-methoxy-1,5-naphthyridin-4-yl)-1-hydroxyethyl)-2-oxabicyclo[2.2.2]octan-4-ylcarbamate (170 mg, Enantiomer A) in the same manner as described for Step 2 of EXAMPLE 1.

¹H NMR (CDCl₃): δ 1.30 (s, 2H), 1.46-1.62 (m, 4H), 1.68-1.81 (m, 3H), 1.86-1.98 (m, 1H), 3.01 (dd, J=12.2, 10.4 Hz, 1H), 3.29-3.32 (m, 1H), 3.43 (s, 2H), 3.73 (ddd, J=9.8, 6.1, 3.1 Hz, 1H), 4.41 (d, J=6.1 Hz, 1H), 7.20 (d, J=9.2 Hz, 1H), 8.25 (d, J=9.2 Hz, 1H), 8.72 (s, 1H).

MS (CI⁺) m/z: 348 (MH⁺).

HRMS (CI⁺) for C₁₈H₂₃FN₃O₃ (MH⁺): calcd, 348.1723. found, 348.1721.

Enantiomer B of 1-(4-amino-2-oxabicyclo[2.2.2]octan-1-yl)-2-(3-fluoro-6-methoxy-1,5-naphthyridin-4-yl)ethanol (100 mg) was prepared in the same manner from tert-butyl 1-(2-(3-fluoro-6-methoxy-1,5-naphthyridin-4-yl)-1-hydroxyethyl)-2-oxabicyclo[2.2.2]octan-4-ylcarbamate (145 mg, Enantiomer B).

¹H NMR (CDCl₃): δ 1.31 (s, 2H), 1.46-1.62 (m, 4H), 1.69-1.81 (m, 3H), 1.89-1.98 (m, 1H), 3.01 (dd, J=12.2, 10.4 Hz, 1H), 3.29-3.37 (m, 1H), 3.43 (s, 2H), 3.73 (ddd, J=9.8, 6.1, 3.1 Hz, 1H), 4.40 (d, J=6.1 Hz, 1H), 7.20 (d, J=9.2 Hz, 1H), 8.25 (d, J=8.6 Hz, 1H), 8.72 (s, 1H).

MS (CI⁺) m/z: 348 (MH⁺).

HRMS (CI⁺) for C₁₈H₂₃FN₃O₃ (MH⁺): calcd, 348.1723. found, 348.1701.

### Step 3

### 6-((1-(2-(3-Fluoro-6-methoxy-1,5-naphthyridin-4-yl)-1-hydroxyethyl)-2-oxabicyclo[2.2.2]octan-4-ylamino)methyl)-2H-pyrido[3,2-b][1,4]oxazin-3(4H)-one (Enantiomer A)

The title compound 6-((1-(2-(3-fluoro-6-methoxy-1,5-naphthyridin-4-yl)-1-hydroxyethyl)-2-oxabicyclo[2.2.2]octan-4-ylamino)methyl)-2H-pyrido[3,2-b][1,4]oxazin-3(4H)-one (120 mg) was prepared from 1-(4-amino-2-oxabicyclo[2.2.2]octan-1-yl)-2-(3-fluoro-6-methoxy-1,5-naphthyridin-4-yl)ethanol (100 mg, Enantiomer A) and I (53.8 mg) in the same manner as described for Step 3 of EXAMPLE 1.

¹H NMR (DMSO-d₆): δ 1.56-2.03 (m, 9H), 3.03 (t, J=10.4 Hz, 1H), 3.29-3.37 (m, 1H), 3.57 (s, 2H), 3.63 (m, 2H), 3.71-3.79 (m, 1H), 4.02 (s, 3H), 4.44 (d, J=6.1 Hz, 1H), 4.59 (s, 2H), 7.01 (d, J=7.9 Hz, 1H), 7.21 (d, J=9.2 Hz, 1H), 7.28 (d, J=7.9 Hz, 1H), 8.25 (d, J=9.2 Hz, 1H), 8.72 (s, 1H), 11.15 (s, 1H).

MS (ESI⁺) m/z: 510 (MH⁺).

HRMS (ESI⁺) for C₂₆H₂₉FN₅O₅ (MH⁺): calcd, 510.21527. found, 510.21492.

Enantiomer B of 6-((1-(2-(3-fluoro-6-methoxy-1,5-naphthyridin-4-yl)-1-hydroxyethyl)-2-oxabicyclo[2.2.2]octan-4-ylamino)methyl)-2H-pyrido[3,2-b][1,4]oxazin-3(4H)-one (114 mg) was prepared in the same manner from 1-(4-amino-2-oxabicyclo[2.2.2]octan-1-yl)-2-(3-fluoro-6-methoxy-1,5-naphthyridin-4-yl)ethanol (90.0 mg, Enantiomer B).

¹H NMR (DMSO-d₆): δ 1.54-2.03 (m, 9H), 3.02 (dd, J=12.2, 11.0 Hz, 1H), 3.28-3.38 (m, 1H), 3.57 (s, 2H), 3.63 (m, 2H), 3.73-3.79 (m, 1H), 4.02 (s, 3H), 4.44 (d, J=6.1 Hz, 1H), 4.59 (s, 2H), 7.01 (d, J=7.9 Hz, 1H), 7.21 (d, J=9.2 Hz, 1H), 7.28 (d, J=7.9 Hz, 1H), 8.26 (d, J=9.2 Hz, 1H), 8.72 (s, 1H), 11.15 (s, 1H).

MS (ESI⁺) m/z: 510 (MH⁺).

HRMS (ESI⁺) for C₂₆H₂₉FN₅O₅ (MH⁺): calcd, 510.21527. found, 510.21587.

### Step 4

### 6-((1-(2-(3-Fluoro-6-methoxy-1,5-naphthyridin-4-yl)-1-hydroxyethyl)-2-oxabicyclo[2.2.2]octan-4-ylamino)methyl)-2H-pyrido[3,2-b][1,4]oxazin-3(4H)-one Hydrochloride (Enantiomer A)

The title compound 6-((1-(2-(3-fluoro-6-methoxy-1,5-naphthyridin-4-yl)-1-hydroxyethyl)-2-oxabicyclo[2.2.2]octan-4-ylamino)methyl)-2H-pyrido[3,2-b][1,4]oxazin-3(4H)-one hydrochloride (200 mg) was prepared from 6-((1-(2-(3-fluoro-6-methoxy-1,5-naphthyridin-4-yl)-1-hydroxyethyl)-2-oxabicyclo[2.2.2]octan-4-ylamino)methyl)-2H-pyrido[3,2-b][1,4]oxazin-3(4H)-one (210 mg, Enantiomer A) in the same manner as described for Step 4 of EXAMPLE 3.

¹H NMR (DMSO-d₆): δ 1.82-2.16 (m, 8H), 3.04 (m, 1H), 3.37 (m, 1H), 3.80 (br, 1H), 3.88 (s, 2H), 4.03 (s, 3H), 4.10 (br, 2H), 4.69 (s, 2H), 4.70 (brs, 1H), 7.20 (br, 1H), 7.22 (d, J=9.2 Hz, 1H), 7.45 (d, J=7.9 Hz, 1H), 8.27 (d, J=9.2 Hz, 1H), 8.74 (s, 1H), 9.26 (br, 2H), 11.32 (s, 1H).

MS (ESI⁺) m/z: 510 (MH⁺) (as free base).

HRMS (ESI⁺) for C₂₆H₂₉FN₅O₅ (MH⁺) (as free base): calcd, 510.21527. found, 510.21491.

Enantiomer B of 6-((1-(2-(3-fluoro-6-methoxy-1,5-naphthyridin-4-yl)-1-hydroxyethyl)-2-oxabicyclo[2.2.2]octan-4-ylamino)methyl)-2H-pyrido[3,2-b][1,4]oxazin-3(4H)-one hydrochloride (219 mg) was prepared in the same manner from 6-((1-(2-(3-fluoro-6-methoxy-1,5-naphthyridin-4-yl)-1-hydroxyethyl)-2-oxabicyclo[2.2.2]octan-4-ylamino)methyl)-2H-pyrido[3,2-b][1,4]oxazin-3(4H)-one (210 mg, Enantiomer B).

¹H NMR (DMSO-d₆): δ 1.81-2.17 (m, 8H), 3.04 (m, 1H), 3.37 (m, 1H), 3.80 (br, 1H), 3.88 (s, 2H), 4.03 (s, 3H), 4.10 (br, 2H), 4.69 (s, 2H), 4.70 (brs, 1H), 7.16-7.20 (m, 1H), 7.22 (d, J=8.6 Hz, 1H), 7.45 (d, J=7.9 Hz, 1H), 8.27 (d, J=9.2 Hz, 1H), 8.74 (s, 1H), 9.27 (br, 2H), 11.32 (s, 1H).

MS (ESI⁺) m/z: 510 (MH⁺) (as free base).

HRMS (ESI⁺) for C₂₆H₂₉FN₅O₅ (MH⁺) (as free base): calcd, 510.21527. found, 510.21453.

### Example 21

### 6-((1-(1-Amino-2-(3-fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one Dihydrochloride (Enantiomer A and Enantiomer B)

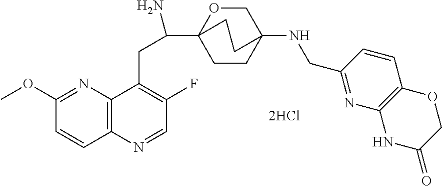 [SMILES: COc1ccc2ncc(F)c(CC(N)C34CCC(NCc5ccc6c(n5)NC(=O)CO6)(CC3)CO4)c2n1 ; "2HCl"]

### Step 1

### tert-Butyl 1-(1-Amino-2-(3-fluoro-6-methoxy-1,5-naphthyridin-4-yl)ethyl)-2-oxabicyclo[2.2.2]octan-4-ylcarbamate

A mixture of S (40.0 mg), ammonium acetate (173 mg) and sodium triacetoxyborohydride (6.54 mg) in methanol (640 μL) and dichloromethane (260 μL) was stirred at room temperature for 6 days and then concentrated in vacuo. After dilution of the residue with dichloromethane, the mixture was washed with saturated sodium hydrogencarbonate solution and brine. The organic extracts were dried over anhydrous sodium sulfate, filtered, and then concentrated in vacuo. Preparative thin layer chromatography (silica, dichloromethane: methanol=10:1) of the residue gave tert-butyl 1-(1-amino-2-(3-fluoro-6-methoxy-1,5-naphthyridin-4-yl)ethyl)-2-oxabicyclo[2.2.2]octan-4-ylcarbamate (26.0 mg).

¹H NMR (DMSO-d₆): δ 1.36 (s, 9H), 1.74-2.00 (m, 8H), 2.83-2.96 (m, 2H), 3.30 (s, 3H), 3.78 (s, 2H), 4.02 (s, 3H), 6.59 (s, 1H), 7.21 (d, J=8.6 Hz, 1H), 8.25 (d, J=9.2 Hz, 1H), 8.73 (s, 1H).

MS (ESI⁺) m/z: 447 (MH⁺).

HRMS (ESI⁺) for C₂₃H₃₂FN₄O₄ (MH⁺): calcd, 447.24076. found, 447.24086.

### Step 2

### tert-Butyl 1-(1-Benzyloxycarbonylamino-2-(3-fluoro-6-methoxy-1,5-naphthyridin-4-yl)ethyl)-2-oxabicyclo[2.2.2]octan-4-ylcarbamate

To a suspension of tert-butyl 1-(1-amino-2-(3-fluoro-6-methoxy-1,5-naphthyridin-4-yl)ethyl)-2-oxabicyclo[2.2.2]octan-4-ylcarbamate (350 mg) in ethyl acetate (2 mL) and sodium hydrogencarbonate solution (316 mg in 3.7 mL of water) was added benzyl chloroformate (134 μL) under cooling with ice, the mixture was stirred at the same temperature for 10 minutes. After dilution of the mixture with water, the mixture was extracted with ethyl acetate. The organic extracts were washed with brine, dried over anhydrous sodium sulfate, filtered, and then concentrated in vacuo. Treatment of the residue with hexaneethyl acetate (2:1) gave tert-butyl 1-(1-benzyloxycarbonylamino-2-(3-fluoro-6-methoxy-1,5-naphthyridin-4-yl)ethyl)-2-oxabicyclo[2.2.2]octan-4-ylcarbamate (398 mg).

¹H NMR (CDCl₃): δ 1.44 (s, 9H), 1.62-1.71 (m, 1H), 1.73-1.94 (m, 3H), 1.98-2.33 (m, 4H), 3.32 (t, J=12.2 Hz, 1H), 3.40-3.53 (m, 1H), 3.90-4.03, (m, 3H), 4.07 (s, 3H), 4.23-4.39 (m, 1H), 4.70 (d, J=12.2 Hz, 1H), 4.76 (d, J=12.8 Hz, 1H), 4.89 (d, J=10.4 Hz, 0.2H), 5.24 (d, J=10.4 Hz, 0.8H), 6.72 (d, J=7.3 Hz, 0.3H), 6.95-7.01 (m, 1.7H), 7.03 (d, J=9.2 Hz, 1H), 7.16-7.30 (m, 3H), 8.09 (d, J=9.2 Hz, 0.1H), 8.14 (d, J=8.6 Hz, 0.9H), 8.53 (s, 1H).

MS (ESI⁺) m/z: 581 (MH⁺).

HRMS (ESI⁺) for C₂₉H₃₂FN₄O₄ (MH⁺): calcd, 581.27754. found, 581.27665.

Optical resolution (CHIRALPAK IA, hexane:IPA:MTBE=85:10:5) of the racemate (380 mg) gave Enantiomer A (183 mg) and Enantiomer B (186 mg).

### Step 3

### Benzyl 1-(4-Amino-2-oxabicyclo[2.2.2]octan-1-yl)-2-(3-fluoro-6-methoxy-1,5-naphthyridin-4-yl)ethylcarbamate (Enantiomer A)

The title compound benzyl 1-(4-amino-2-oxabicyclo[2.2.2]octan-1-yl)-2-(3-fluoro-6-methoxy-1,5-naphthyridin-4-yl)ethylcarbamate (131 mg) was prepared from tert-butyl 1-(1-benzyloxycarbonylamino-2-(3-fluoro-6-methoxy-1,5-naphthyridin-4-yl)ethyl)-2-oxabicyclo[2.2.2]octan-4-ylcarbamate (170 mg, Enantiomer A) in the same manner as described for Step 2 of EXAMPLE 1.

¹H NMR (DMSO-d₆): δ 0.94-1.08 (brs, 2H), 1.46-1.89 (m, 6H), 2.04-2.14 (m, 1H), 2.21-2.31 (m, 1H), 3.26-3.36 (m, 1H), 3.46-3.54 (m, 1H), 3.61-3.71 (m, 2H), 3.97-4.06 (m, 1H), 4.08 (s, 3H), 4.70 (d, J=12.9 Hz, 1H), 4.76 (d, J=12.2 Hz, 1H), 5.25 (d, J=9.8 Hz, 1H), 6.71 (d, J=6.7 Hz, 0.2H), 6.94-7.02 (m, 1.8H), 7.03 (d, J=9.2 Hz, 1H), 7.16-7.33 (m, 3H), 8.09 (d, J=9.2 Hz, 0.2H), 8.14 (d, J=9.2 Hz, 0.8H), 8.53 (s, 1H).

MS (ESI⁺) m/z: 481 (MH⁺).

HRMS (ESI⁺) for C₂₆H₃₀FN₄O₄ (MH⁺): calcd, 481.22511. found, 481.22500.

Enantiomer B of benzyl 1-(4-amino-2-oxabicyclo[2.2.2]octan-1-yl)-2-(3-fluoro-6-methoxy-1,5-naphthyridin-4-yl)ethylcarbamate (132 mg) was prepared in the same manner from tert-butyl 1-(1-benzyloxycarbonylamino-2-(3-fluoro-6-methoxy-1,5-naphthyridin-4-yl)ethyl)-2-oxabicyclo[2.2.2]octan-4-ylcarbamate (170 mg, Enantiomer B).

¹H NMR (DMSO-d₆): δ 0.93-1.13 (brs, 2H), 1.46-1.88 (m, 6H), 2.05-2.14 (m, 1H), 2.20-2.32 (m, 1H), 3.26-3.36 (m, 1H), 3.46-3.54 (m, 1H), 3.61-3.71 (m, 2H), 3.97-4.14 (m, 1H), 4.08 (s, 3H), 4.70 (d, J=12.2 Hz, 1H), 4.76 (d, J=12.2 Hz, 1H), 5.25 (d, J=9.8 Hz, 1H), 6.71 (d, J=6.7 Hz, 0.3H), 6.94-7.01 (m, 1.7H), 7.03 (d, J=8.6 Hz, 1H), 7.16-7.33 (m, 3H), 8.09 (d, J=9.2 Hz, 0.2H), 8.14 (d, J=9.2 Hz, 0.8H), 8.53 (s, 1H).

MS (ESI⁺) m/z: 481 (MH⁺).

HRMS (ESI⁺) for C₂₆H₃₀FN₄O₄ (MH⁺): calcd, 481.22511. found, 481.22522.

### Step 4

### Benzyl 2-(3-Fluoro-6-methoxy-1,5-naphthyridin-4-yl)-1-(4-((3-oxo-3,4-dihydro-2H-pyrido[3,2-b][1,4]oxazin-6-yl)methylamino)-2-oxabicyclo[2.2.2]octan-1-yl)ethylcarbamate (Enantiomer A)

The title compound benzyl 2-(3-fluoro-6-methoxy-1,5-naphthyridin-4-yl)-1-(4-((3-oxo-3,4-dihydro-2H-pyrido[3,2-b][1,4]oxazin-6-yl)methylamino)-2-oxabicyclo[2.2.2]octan-1-yl)ethylcarbamate (121 mg) was prepared from benzyl 1-(4-amino-2-oxabicyclo[2.2.2]octan-1-yl)-2-(3-fluoro-6-methoxy-1,5-naphthyridin-4-yl)ethylcarbamate (100 mg, Enantiomer A) and I (36.9 mg) in the same manner as described for Step 3 of EXAMPLE 1.

¹H NMR (CDCl₃): δ 1.58-1.91 (m, 6H), 2.12-2.34 (m, 2H), 3.27-3.37 (m, 1H), 3.44-3.55 (m, 1H), 3.73-3.81 (m, 4H), 3.98-4.06 (m, 1H), 4.07 (s, 3H), 4.64 (s, 2H), 4.73 (q, J=12.6 Hz, 2H), 5.27 (d, J=9.8 Hz, 1H), 6.71 (d, J=7.3 Hz, 0.3H), 6.93-7.29 (m, 9H), 8.08-8.16 (m, 1.7H), 8.54 (s, 1H).

MS (ESI⁺) m/z: 643 (MH⁺).

HRMS (ESI⁺) for C₃₄H₃₆FN₆O₆ (MH⁺): calcd, 643.26803. found, 643.26717.

Enantiomer B of benzyl 2-(3-fluoro-6-methoxy-1,5-naphthyridin-4-yl)-1-(4-((3-oxo-3,4-dihydro-2H-pyrido[3,2-b][1,4]oxazin-6-yl)methylamino)-2-oxabicyclo[2.2.2]octan-1-yl)ethylcarbamate (117 mg) was prepared in the same manner from benzyl 1-(4-amino-2-oxabicyclo[2.2.2]octan-1-yl)-2-(3-fluoro-6-metho xy-1,5-naphthyridin-4-yl)ethylcarbamate (100 mg, Enantiomer B) and I (36.9 mg).

¹H NMR (CDCl₃): δ 1.63-1.90 (m, 6H), 2.07-2.35 (m, 2H), 3.26-3.37 (m, 1H), 3.46-3.55 (m, 1H), 3.72-3.82 (m, 4H), 3.98-4.06 (m, 1H), 4.08 (s, 3H), 4.64 (s, 2H), 4.73 (q, J=12.9 Hz, 2H), 5.26 (d, J=10.3 Hz, 1H), 6.71 (d, J=6.1 Hz, 0.3H), 6.93-7.30 (m, 9H), 7.94-8.16 (m, 1.7H), 8.54 (s, 1H).

MS (ESI⁺) m/z: 643 (MH⁺).

HRMS (ESI⁺) for C₃₄H₃₆FN₆O₆ (MH⁺): calcd, 643.26803. found, 643.26728.

### Step 5

### 6-((1-(1-Amino-2-(3-fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one (Enantiomer A)

The title compound 6-((1-(1-amino-2-(3-fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one (70.0 mg) was prepared from benzyl 2-(3-fluoro-6-methoxy-1,5-naphthyridin-4-yl)-1-(4-((3-oxo-3,4-dihydro-2H-pyrido[3,2-b][1,4]oxazin-6-yl)methylamino)-2-oxabicyclo[2.2.2]octan-1-yl)ethylcarbamate (100 mg, Enantiomer A) in the same manner as described for Step 4 of EXAMPLE 38.

¹H NMR (DMSO-d₆): δ 1.14 (brs, 2H), 1.57-1.99 (m, 9H), 2.85-2.96 (m, 2H), 3.22-3.41 (m, 1H), 3.58 (s, 2H), 3.63 (s, 2H), 4.02 (s, 3H), 4.59 (s, 2H), 7.01 (d, J=8.6 Hz, 1H), 7.21 (d, J=9.2 Hz, 1H), 7.28 (d, J=8.0 Hz, 1H), 8.25 (d, J=8.6 Hz, 1H), 8.73 (s, 1H), 11.15 (s, 1H).

MS (ESI⁺) m/z: 509 (MH⁺).

HRMS (ESI⁺) for C₂₆H₃₀FN₆O₄ (MH⁺): calcd, 509.23126. found, 509.23213.

Enantiomer B of 6-((1-(1-amino-2-(3-fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one (71.5 mg) was prepared in the same manner from benzyl 2-(3-fluoro-6-methoxy-1,5-naphthyridin-4-yl)-1-(4-((3-oxo-3,4-dihydro-2H-pyrido[3,2-b][1,4]oxazin-6-yl)methylamino)-2-oxabicyclo[2.2.2]octan-1-yl)ethylcarbamate (100 mg, Enantiomer B).

¹H NMR (DMSO-d₆): δ 1.56-1.99 (m, 9H), 2.85-2.98 (m, 2H), 3.27-3.36 (m, 1H), 3.59 (s, 2H), 3.63 (s, 2H), 4.02 (s, 3H), 4.59 (s, 2H), 7.01 (d, J=8.6 Hz, 1H), 7.21 (d, J=9.2 Hz, 1H), 7.28 (d, J=7.9 Hz, 1H), 8.26 (d, J=8.6 Hz, 1H), 8.74 (s, 1H), 11.16 (s, 1H).

MS (ESI⁺) m/z: 509 (MH⁺).

HRMS (ESI⁺) for C₂₆H₃₀FN₆O₄ (MH⁺): calcd, 509.23126. found, 509.23207.

### Step 6

### 6-((1-(1-Amino-2-(3-fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one Hydrochloride (Enantiomer A)

The title compound 6-((1-(1-amino-2-(3-fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one hydrochloride (Enantiomer A) (63.0 mg) was prepared from 6-((1-(1-amino-2-(3-fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one (60.0 mg) in the same manner as described for Step 4 of EXAMPLE 3.

¹H NMR (DMSO-d₆): δ 1.92-2.14 (m, 8H), 3.12-3.21 (m, 1H), 3.42-3.54 (m, 1H), 3.63-3.72 (m, 1H), 3.95-4.03 (m, 2H), 4.06 (s, 3H), 4.03-4.14 (m, 2H), 4.69 (s, 2H), 7.28 (d, J=8.6 Hz, 1H), 7.30 (d, J=8.0 Hz, 1H), 7.45 (d, J=8.0 Hz, 1H), 8.00 (brs, 3H), 8.33 (d, J=9.2 Hz, 1H), 8.83 (s, 1H), 9.68 (brs, 2H), 11.32 (s, 1H).

MS (ESI⁺) m/z: 509 (MH⁺) (as free base).

HRMS (ESI⁺) for C₂₆H₃₀FN₆O₄ (MH⁺) (as free base): calcd, 509.23126. found, 509.23204.

Enantiomer B of 6-((1-(1-amino-2-(3-fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one hydrochloride (57.8 mg) was prepared in the same manner from 6-((1-(1-amino-2-(3-fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one (60.0 mg, Enantiomer B).

¹H NMR (DMSO-d₆): δ 1.93-2.25 (m, 8H), 3.12-3.21 (m, 1H), 3.48-3.56 (m, 1H), 3.63-3.71 (m, 1H), 3.96-4.04 (m, 2H), 4.06 (s, 3H), 4.05-4.14 (m, 2H), 4.69 (s, 2H), 7.28 (d, J=9.2 Hz, 1H), 7.30 (d, J=8.6 Hz, 1H), 7.45 (d, J=8.0 Hz, 1H), 7.99 (brs, 3H), 8.33 (d, J=9.2 Hz, 1H), 8.83 (s, 1H), 9.67 (brs, 2H), 11.31 (s, 1H).

MS (ESI⁺) m/z: 509 (MH⁺) (as free base).

HRMS (ESI⁺) for C₂₆H₃₀FN₆O₄ (MH⁺) (as free base): calcd, 509.23126. found, 509.23115.

### Example 22

### 6-((1-(2-(3-Fluoro-6-methoxy-1,5-naphthyridin-4-yl)-1-methoxyethyl)-2-oxabicyclo[2.2.2]octan-4-ylamino)methyl)-2H-pyrido[3,2-b][1,4]oxazin-3(4H)-one Hydrochloride

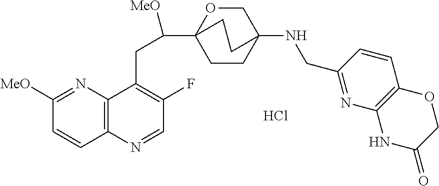 [SMILES: COc1ccc2ncc(F)c(CC(OC)C34CCC(NCc5ccc6c(n5)NC(=O)CO6)(CC3)CO4)c2n1 ; Cl]

### Step 1

### tert-Butyl 1-(2-(3-Fluoro-6-methoxy-1,5-naphthyridin-4-yl)-1-methoxyethyl)-2-oxabicyclo[2.2.2]octan-4-ylcarbamate

To a suspension of sodium hydride (42.9 mg, 55% in mineral oil) in N,N-dimethylformamide (3.5 mL) was added a solution of tert-butyl 1-(2-(3-fluoro-6-methoxy-1,5-naphthyridin-4-yl)-1-hydroxyethyl)-2-oxabicyclo[2.2.2]octan-4-ylcarbamate (200 mg, Enantiomer A) in N,N-dimethylformamide (0.6 mL) at −40° C., the mixture was stirred at −20° C. for 2 hours. Methyl benzenesulfonate (66.7 μL) was added to the mixture. The mixture was stirred under cooling with ice for 2.5 hours. After dilution of the mixture with water, the mixture was extracted with ethyl acetate. The organic extracts were washed with brine, dried over anhydrous sodium sulfate, filtered, and then concentrated in vacuo. Preparative thin layer chromatography (silica, toluene:methanol=7:1) of the residue gave tert-butyl 1-(2-(3-fluoro-6-methoxy-1,5-naphthyridin-4-yl)-1-methoxyethyl)-2-oxabicyclo[2.2.2]octan-4-ylcarbamate (125 mg).

¹H NMR (CDCl₃): δ 1.42 (s, 9H), 1.78-1.94 (m, 4H), 1.97-2.23 (m, 4H), 3.08 (s, 3H), 3.28 (dd, J=12.7, 3.6 Hz, 1H), 3.42 (ddd, J=12.7, 4.2, 1.8 Hz, 1H), 3.61 (dd, J=9.1, 3.6 Hz, 1H), 3.86-3.94 (m, 2H), 4.09 (s, 3H), 4.28 (brs, 1H), 7.07 (d, J=9.1 Hz, 1H), 8.17 (d, J=9.1 Hz, 1H), 8.62 (s, 1H).

MS (ESI⁺) m/z: 462 (MH⁺).

HRMS (ESI⁺) for C₂₄H₃₃FN₃O₅ (MH⁺): calcd, 462.24042. found, 462.23972.

### Step 2

### 1-(2-(3-Fluoro-6-methoxy-1,5-naphthyridin-4-yl)-1-methoxyethyl)-2-oxabicyclo[2.2.2]octan-4-amine

The title compound 1-(2-(3-fluoro-6-methoxy-1,5-naphthyridin-4-yl)-1-methoxyethyl)-2-oxabicyclo[2.2.2]octan-4-amine (52.2 mg) was prepared from tert-butyl 1-(2-(3-fluoro-6-methoxy-1,5-naphthyridin-4-yl)-1-methoxyethyl)-2-oxabicyclo[2.2.2]octan-4-ylcarbamate (80.0 mg) in the same manner as described for Step 2 of EXAMPLE 1.

¹H NMR (CDCl₃): δ 1.60-1.93 (m, 6H), 1.98-2.06 (m, 1H), 2.13-2.22 (m, 1H), 3.07 (s, 3H), 3.29 (dd, J=12.7, 9.1 Hz, 1H), 3.42 (ddd, J=12.7, 4.2, 1.8 Hz, 1H), 3.57 (s, 2H), 3.61 (dd, J=9.1, 4.2 Hz, 1H), 4.09 (s, 3H), 7.07 (d, J=9.1 Hz, 1H), 8.18 (d, J=9.1 Hz, 1H), 8.62 (s, 1H).

MS (ESI⁺) m/z: 362 (MH⁺).

HRMS (ESI⁺) for C₁₉H₂₅FN₃O₃ (MH⁺): calcd, 362.18799. found, 362.18769.

### Step 3

### 6-(((1-(2-(3-Fluoro-6-methoxy-1,5-naphthyridin-4-yl)-1-methoxyethyl)-2-oxabicyclo[2.2.2]octan-4-yl)amino)methyl)-2H-pyrido[3,2-b][1,4]oxazin-3(4H)-one

The title compound 6-(((1-(2-(3-fluoro-6-methoxy-1,5-naphthyridin-4-yl)-1-methoxyethyl)-2-oxabicyclo[2.2.2]octan-4-yl)amino)methyl)-2H-pyrido[3,2-b][1,4]oxazin-3(4H)-one (55.7 mg) was prepared from 1-(2-(3-fluoro-6-methoxy-1,5-naphthyridin-4-yl)-1-methoxyethyl)-2-oxabicyclo[2.2.2]octan-4-amine (50.0 mg) and I (25.9 mg) in the same manner as described for Step 3 of EXAMPLE 1.

¹H NMR (DMSO-d₆): δ 1.55-1.92 (m, 8H), 1.95-2.07 (m, 1H), 2.94 (s, 3H), 3.15 (dd, J=12.2, 9.2 Hz, 1H), 3.29-3.38 (m, 1H), 3.50 (s, 2H), 3.55 (dd, J=9.2, 4.3 Hz, 1H), 3.60 (s, 2H), 4.04 (s, 3H), 4.58 (s, 2H), 6.99 (d, J=7.9 Hz, 1H), 7.23 (d, J=9.2 Hz, 1H), 7.27 (d, J=7.9 Hz, 1H), 8.27 (d, J=9.2 Hz, 1H), 8.75 (s, 1H), 11.15 (s, 1H).

MS (ESI⁺) m/z: 524 (MH⁺).

HRMS (ESI⁺) for C₂₇H₃₁FN₅O₅ (MH⁺): calcd, 524.23092. found, 524.23092.

### Step 4

### 6-(((1-(2-(3-Fluoro-6-methoxy-1,5-naphthyridin-4-yl)-1-methoxyethyl)-2-oxabicyclo[2.2.2]octan-4-yl)amino)methyl)-2H-pyrido[3,2-b][1,4]oxazin-3(4H)-one Hydrochloride

The title compound 6-(((1-(2-(3-fluoro-6-methoxy-1,5-naphthyridin-4-yl)-1-methoxyethyl)-2-oxabicyclo[2.2.2]octan-4-yl)amino)methyl)-2H-pyrido[3,2-b][1,4]oxazin-3(4H)-one hydrochloride (48.4 mg) was prepared from 6-(((1-(2-(3-fluoro-6-methoxy-1,5-naphthyridin-4-yl)-1-methoxyethyl)-2-oxabicyclo[2.2.2]octan-4-yl)amino)methyl)-2H-pyrido[3,2-b][1,4]oxazin-3(4H)-one (50.0 mg) in the same manner as described for Step 4 of EXAMPLE 3.

¹H NMR (DMSO-d₆): δ 1.84-2.17 (m, 8H), 2.98 (s, 3H), 3.17 (dd, J=12.2, 9.2 Hz, 1H), 3.39 (dd, J=12.8, 9.8 Hz, 1H), 3.61 (dd, J=9.2, 4.3 Hz, 1H), 3.81 (s, 2H), 4.04 (s, 3H), 4.09 (d, J=6.1 Hz, 2H), 4.68 (s, 2H), 7.20 (d, J=7.9 Hz, 1H), 7.25 (d, J=8.6 Hz, 1H), 7.45 (d, J=7.9 Hz, 1H), 8.29 (d, J=9.2 Hz, 1H), 8.77 (s, 1H), 9.28 (brs, 2H), 11.32 (s, 1H).

MS (ESI⁺) m/z: 524 (MH⁺) (as free base).

HRMS (ESI⁺) for C₂₇H₃₁FN₅O₅ (MH⁺) (as free base): calcd, 524.23092. found, 524.23153.

### Example 23

### 6-((1-(2-(3-Fluoro-6-methoxy-1,5-naphthyridin-4-yl)acetyl)-2-oxabicyclo[2.2.2]octan-4-ylamino)methyl)-2H-pyrido[3,2-b][1,4]oxazin-3(4H)-one

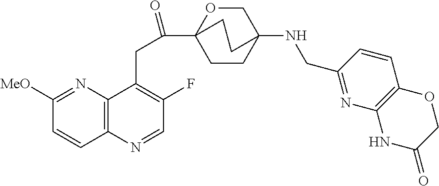 [SMILES: COc1ccc2ncc(F)c(CC(=O)C34CCC(NCc5ccc6c(n5)NC(=O)CO6)(CC3)CO4)c2n1]

### Step 1

### 1-(4-Amino-2-oxabicyclo[2.2.2]octan-1-yl)-2-(3-fluoro-6-methoxy-1,5-naphthyridin-4-yl)ethanone

The title compound 1-(4-amino-2-oxabicyclo[2.2.2]octan-1-yl)-2-(3-fluoro-6-methoxy-1,5-naphthyridin-4-yl)ethanone (23.2 mg) was prepared from S (30.0 mg) in the same manner as described for Step 2 of EXAMPLE 1.

¹H NMR (CDCl₃): δ 1.22 (brs, 2H), 1.66-1.84 (m, 4H), 1.98-2.18 (m, 4H), 3.81 (s, 2H), 3.99 (s, 3H), 4.55 (s, 2H), 7.05 (d, J=9.2 Hz, 1H), 8.18 (d, J=9.2 Hz, 1H), 8.65 (s, 1H).

MS (ESI⁺) m/z: 346 (MH⁺).

HRMS (ESI⁺) for C₁₈H₂₁FN₃O₃ (MH⁺): calcd, 346.15669. found, 346.15730.

### Step 2

### 6-((1-(2-(3-Fluoro-6-methoxy-1,5-naphthyridin-4-yl)acetyl)-2-oxabicyclo[2.2.2]octan-4-ylamino)methyl)-2H-pyrido[3,2-b][1,4]oxazin-3(4H)-one

A mixture of 1-(4-amino-2-oxabicyclo[2.2.2]octan-1-yl)-2-(3-fluoro-6-methoxy-1,5-naphthyridin-4-yl)ethanone (140 mg), T (77.8 mg) and diisopropylethylamine (102 μL) in N,N-dimethylformamide (2.3 mL) was stirred at room temperature for 112 hours and then concentrated in vacuo. After dilution of the residue with dichloromethane, the mixture was washed with saturated sodium hydrogencarbonate solution, water and brine, dried over anhydrous sodium sulfate, filtered, and then concentrated in vacuo. Preparative thin layer chromatography (silica, chloroform:methanol=10:1) of the residue gave 6-((1-(2-(3-fluoro-6-methoxy-1,5-naphthyridin-4-yl)acetyl)-2-oxabicyclo[2.2.2]octan-4-ylamino)methyl)-2H-pyrido[3,2-b][1,4]oxazin-3(4H)-one.

¹H NMR (DMSO-d₆): δ 1.65-1.82 (m, 4H), 1.84-1.95 (m, 2H), 1.98-2.09 (m, 3H), 3.65 (d, J=6.1 Hz, 2H), 3.79 (s, 2H), 3.96 (s, 3H), 4.50 (s, 2H), 4.59 (s, 2H), 7.02 (d, J=8.0 Hz, 1H), 7.23 (d, J=9.2 Hz, 1H), 7.29 (d, J=8.0 Hz, 1H), 8.29 (d, J=9.2 Hz, 1H), 8.81 (s, 1H), 11.16 (s, 1H).

MS (ESI⁺) m/z: 508 (MH⁺).

HRMS (ESI⁺) for C₂₆H₂₇FN₅O₅ (MH⁺): calcd, 508.19962. found, 508.19896.

### Example 24

### 3-((1-(2-(3-Fluoro-6-methoxy-1,5-naphthyridin-4-yl)ethyl)-2-oxabicyclo[2.2.2]octan-4-ylamino)methyl)-1-methylquinoxalin-2(1H)-one

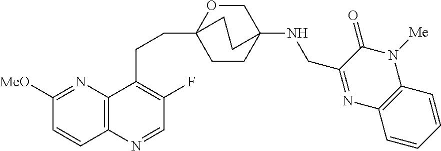 [SMILES: COc1ccc2ncc(F)c(CCC34CCC(NCc5nc6ccccc6n(C)c5=O)(CC3)CO4)c2n1]

The title compound 3-((1-(2-(3-fluoro-6-methoxy-1,5-naphthyridin-4-yl)ethyl)-2-oxabicyclo[2.2.2]octan-4-ylamino)methyl)-1-methylquinoxalin-2(1H)-one (38.0 mg) was prepared from 1-(2-(3-fluoro-6-methoxy-1,5-naphthyridin-4-yl)ethyl)-2-oxabicyclo[2.2.2]octan-4-amine (50.0 mg) and commercially available 3-(bromomethyl)-1-methylquinoxalin-2(1H)-one (38.2 mg) in the same manner as described for Step 2 of EXAMPLE 23.

¹H NMR (DMSO-d₆): δ 1.58-1.91 (m, 10H), 2.05 (br, 1H), 3.05-3.18 (m, 2H), 3.63 (s, 5H), 3.88 (s, 2H), 4.03 (s, 3H), 7.22 (d, J=9.2 Hz, 1H), 7.34-7.41 (m, 1H), 7.54-7.65 (m, 2H), 7.82 (d, J=8.0 Hz, 1H), 8.26 (d, J=9.2 Hz, 1H), 8.74 (s, 1H).

MS (ESI⁺) m/z: 504 (MH⁺).

HRMS (ESI⁺) for C₂₈H₃₁FN₅O₃ (MH⁺): calcd, 504.24109. found, 504.24167.

### Example 25

### 6-((1-(2-(3-Fluoro-6-methoxy-1,5-naphthyridin-4-yl)-1-(hydroxyimino)ethyl)-2-oxabicyclo[2.2.2]octan-4-ylamino)methyl)-2H-pyrido[3,2-b][1,4]oxazin-3(4H)-one (isomer A and isomer B)

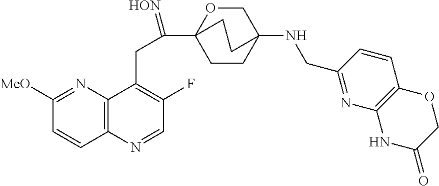

The title compound 6-((1-(2-(3-fluoro-6-methoxy-1,5-naphthyridin-4-yl)-1-(hydroxyimino)ethyl)-2-oxabicyclo[2.2.2]octan-4-ylamino)methyl)-2H-pyrido[3,2-b][1,4]oxazin-3(4H)-one [Isomer A (22.4 mg) and Isomer B (38.7 mg)] was prepared from 6-((1-(2-(3-fluoro-6-methoxy-1,5-naphthyridin-4-yl)acetyl)-2-oxabicyclo[2.2.2]octan-4-ylamino)methyl)-2H-pyrido[3,2-b][1,4]oxazin-3(4H)-one (EXAMPLE 23, 65.0 mg) and hydroxylamine hydrochloride (35.6 mg) in pyridine (7.4 mL) was heated at 80° C. for 51 hours and then concentrated in vacuo. After dilution of the residue with dichloromethane, the mixture was washed with water and brine, dried over anhydrous sodium sulfate, filtered, and then concentrated in vacuo. Preparative thin layer chromatography (silica, chloroform:methanol=10:1) of the residue gave, Isomer A: ¹H NMR (DMSO-d₆): δ 1.55-2.13 (m, 7H), 2.59-2.71 (m, 2H), 3.64 (s, 2H), 3.71 (s, 2H), 3.99 (s, 3H), 4.07 (s, 2H), 4.59 (s, 2H), 7.01 (d, J=8.0 Hz, 1H), 7.20 (d, J=9.2 Hz, 1H), 7.28 (d, J=8.0 Hz, 1H), 8.24 (d, J=8.6 Hz, 1H), 8.73 (s, 1H), 10.55 (s, 1H), 11.15 (s, 1H).

MS (ESI⁺) m/z: 523 (MH⁺).

HRMS (ESI⁺) for C₂₆H₂₈FN₆O₅ (MH⁺): calcd, 523.21052. found, 523.21148.

Isomer B: ¹H NMR (DMSO-d₆): δ 1.47-1.58 (m, 2H), 1.61-1.72 (m, 2H), 1.75-1.91 (m, 3H), 1.96-2.09 (m, 2H), 3.36 (s, 2H), 3.55 (d, J=6.1 Hz, 2H), 4.03 (s, 3H), 4.18 (s, 2H), 4.57 (s, 2H), 6.95 (d, J=8.0 Hz, 1H), 7.21 (d, J=9.2 Hz, 1H), 7.25 (d, J=8.0 Hz, 1H), 8.25 (d, J=9.2 Hz, 1H), 8.66 (s, 1H), 10.75 (s, 1H), 11.12 (s, 1H).

MS (ESI⁺) m/z: 523 (MH⁺).

HRMS (ESI⁺) for C₂₆H₂₈FN₆O₅ (MH⁺): calcd, 523.21052. found, 523.21114.

### Example 26

### 6-((1-(2-(3-Fluoro-6-methoxy-1,5-naphthyridin-4-yl)-1,2-dihydroxyethyl)-2-oxabicyclo[2.2.2]octan-4-ylamino)methyl)-2H-pyrido[3,2-b][1,4]oxazin-3(4H)-one Hydrochloride (Enantiomer A)

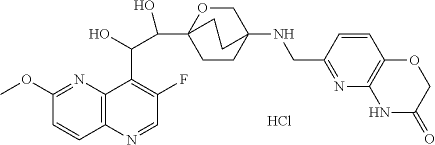 [SMILES: COc1ccc2ncc(F)c(C(O)C(O)C34CCC(NCc5ccc6c(n5)NC(=O)CO6)(CC3)CO4)c2n1 ; Cl]

### Step 1

### tert-Butyl 1-(2-(3-Fluoro-6-methoxy-1,5-naphthyridin-4-yl)-1,2-dihydroxyethyl)-2-oxabicyclo[2.2.2]octan-4-ylcarbamate (Enantiomer A and Enantiomer B)

A mixture of U (100 mg), osmium tetroxide solution (118 μL, 2.5 wt % in tert-butanol) and 4-methylmorpholine N-oxide solution (146 μL, 50 wt % in water) in tert-butanol (1.7 mL) and water (0.17 mL) was stirred at room temperature for 5 hours. After dilution of the mixture with water, the mixture was added sodium hydrogen sulfite (0.14 g). The mixture was extracted with ethyl acetate. The organic extracts were dried over anhydrous sodium sulfate, filtered, and then concentrated in vacuo. Flash chromatography of the residue gave tert-butyl 1-(2-(3-fluoro-6-methoxy-1,5-naphthyridin-4-yl)-1,2-dihydroxyethyl)-2-oxabicyclo[2.2.2]octan-4-ylcarbamate.

¹H NMR (CDCl₃): δ 1.42 (s, 9H), 1.77-2.30 (m, 8H), 3.68-3.73 (m, 2H), 3.82-3.98 (m, 2H), 4.06 (s, 3H), 4.28 (brs, 1H), 5.68 (dd, J=8.6, 3.1 Hz, 1H), 5.78 (d, J=7.9 Hz, 1H), 7.10 (d, J=8.6 Hz, 1H), 8.23 (d, J=9.2 Hz, 1H), 8.65 (d, J=1.2 Hz, 1H).

MS (ESI⁺) m/z: 464 (MH⁺).

HRMS (ESI⁺) for C₂₃H₃₁FN₃O₆ (MH⁺): calcd, 464.21969. found, 464.22023.

### Step 2

### 1-(4-Amino-2-oxabicyclo[2.2.2]octan-1-yl)-2-(3-fluoro-6-methoxy-1,5-naphthyridin-4-yl)ethane-1,2-diol (Enantiomer A)

The title compound 1-(4-amino-2-oxabicyclo[2.2.2]octan-1-yl)-2-(3-fluoro-6-methoxy-1,5-naphthyridin-4-yl)ethane-1,2-diol (140 mg, Enantiomer A) was prepared from tert-butyl 1-(2-(3-fluoro-6-methoxy-1,5-naphthyridin-4-yl)-1,2-dihydroxyethyl)-2-oxabicyclo[2.2.2]octan-4-ylcarbamate (195 mg, Enantiomer A) in the same manner as described for Step 2 of EXAMPLE 1.

¹H NMR (CDCl₃): δ 1.40-2.27 (m, 8H), 3.51-3.63 (m, 2H), 3.65-3.82 (m, 2H), 4.06 (s, 3H), 5.73 (q, J=3.5 Hz, 1H), 5.79 (d, J=7.9 Hz, 1H), 7.11 (d, J=9.2 Hz, 1H), 8.23 (d, J=9.2 Hz, 1H), 8.65 (d, J=1.2 Hz, 1H).

MS (ESI⁺) m/z: 364 (MH⁺).

HRMS (ESI⁺) for C₁₈H₂₃FN₃O₄ (MH⁺): calcd, 364.16726. found, 364.16631.

Enantiomer B of 1-(4-amino-2-oxabicyclo[2.2.2]octan-1-yl)-2-(3-fluoro-6-methoxy-1,5-naphthyridin-4-yl)ethane-1,2-diol (142 mg) was prepared in the similar manner from tert-butyl 1-(2-(3-fluoro-6-methoxy-1,5-naphthyridin-4-yl)-1,2-dihydroxyethyl)-2-oxabicyclo[2.2.2]octan-4-ylcarbamate (195 mg, Enantiomer B).

¹H NMR (CDCl₃): δ 1.40-2.27 (m, 8H), 3.51-3.63 (m, 2H), 3.65-3.82 (m, 2H), 4.06 (s, 3H), 5.73 (q, J=3.5 Hz, 1H), 5.79 (d, J=7.9 Hz, 1H), 7.11 (d, J=9.2 Hz, 1H), 8.23 (d, J=9.2 Hz, 1H), 8.65 (d, J=1.2 Hz, 1H).

MS (ESI⁺) m/z: 364 (MH⁺).

HRMS (ESI⁺) for C₁₈H₂₃FN₃O₄ (MH⁺): calcd, 364.16726. found, 364.16759.

### Step 3

### 6-((1-(2-(3-Fluoro-6-methoxy-1,5-naphthyridin-4-yl)-1,2-dihydroxyethyl)-2-oxabicyclo[2.2.2]octan-4-ylamino)methyl)-2H-pyrido[3,2-b][1,4]oxazin-3(4H)-one (Enantiomer A)

The title compound 6-((1-(2-(3-fluoro-6-methoxy-1,5-naphthyridin-4-yl)-1,2-dihydroxyethyl)-2-oxabicyclo[2.2.2]octan-4-ylamino)methyl)-2H-pyrido[3,2-b][1,4]oxazin-3(4H)-one (156 mg) was prepared from 1-(4-amino-2-oxabicyclo[2.2.2]octan-1-yl)-2-(3-fluoro-6-methoxy-1,5-naphthyridin-4-yl)ethane-1,2-diol (130 mg, Enantiomer A) and I (66.9 mg) in the same manner as described for Step 3 of EXAMPLE 1.

¹H NMR (DMSO-d₆): δ 1.36-2.00 (m, 8H), 2.14 (brs, 1H), 2.88-3.25 (m, 2H), 3.51 (brs, 2H), 3.64 (t, J=5.8 Hz, 1H), 4.03 (s, 3H), 4.57 (s, 2H), 5.00 (d, J=5.5 Hz, 1H), 5.39 (d, J=6.7 Hz, 1H), 5.78 (d, J=6.1 Hz, 1H), 6.93 (d, J=8.6 Hz, 1H), 7.22 (d, J=9.2 Hz, 1H), 7.23 (d, J=9.2 Hz, 1H), 8.26 (d, J=9.2 Hz, 1H), 8.70 (d, J=1.8 Hz, 1H), 11.11 (s, 1H).

MS (ESI⁺) m/z: 526 (MH⁺).

HRMS (ESI⁺) for C₂₆H₂₉FN₅O₆ (MH⁺): calcd, 526.21019. found, 526.20974.

Enantiomer B of 6-((1-(2-(3-fluoro-6-methoxy-1,5-naphthyridin-4-yl)-1,2-dihydroxyethyl)-2-oxabicyclo[2.2.2]octan-4-ylamino)methyl)-2H-pyrido[3,2-b][1,4]oxazin-3(4H)-one (138 mg) was prepared in the similar manner from 1-(4-amino-2-oxabicyclo[2.2.2]octan-1-yl)-2-(3-fluoro-6-metho xy-1,5-naphthyridin-4-yl)ethane-1,2-diol (130 mg, Enantiomer B).

¹H NMR (DMSO-d₆): δ 1.36-2.00 (m, 8H), 2.14 (brs, 1H), 2.95-3.26 (m, 2H), 3.51 (s, 2H), 3.64 (t, J=5.5 Hz, 1H), 4.03 (s, 3H), 4.57 (s, 2H), 5.01 (d, J=6.1 Hz, 1H), 5.39 (d, J=6.7 Hz, 1H), 5.78 (t, J=6.1 Hz, 1H), 6.93 (d, J=7.9 Hz, 1H), 7.22 (d, J=9.2 Hz, 1H), 7.23 (d, J=8.6 Hz, 1H), 8.26 (d, J=9.2 Hz, 1H), 8.70 (d, J=1.8 Hz, 1H), 11.11 (s, 1H).

MS (ESI⁺) m/z: 526 (MH⁺).

HRMS (ESI⁺) for C₂₆H₂₉FN₅O₆ (MH⁺): calcd, 526.21019. found, 526.21068.

### Step 4

### 6-((1-(2-(3-Fluoro-6-methoxy-1,5-naphthyridin-4-yl)-1,2-dihydroxyethyl)-2-oxabicyclo[2.2.2]octan-4-ylamino)methyl)-2H-pyrido[3,2-b][1,4]oxazin-3(4H)-one Hydrochloride (Enantiomer A)

The title compound 6-((1-(2-(3-fluoro-6-methoxy-1,5-naphthyridin-4-yl)-1,2-dihydroxyethyl)-2-oxabicyclo[2.2.2]octan-4-ylamino)methyl)-2H-pyrido[3,2-b][1,4]oxazin-3(4H)-one hydrochloride (108 mg) was prepared from 6-((1-(2-(3-fluoro-6-methoxy-1,5-naphthyridin-4-yl)-1,2-dihydroxyethyl)-2-oxabicyclo[2.2.2]octan-4-ylamino)methyl)-2H-pyrido[3,2-b][1,4]oxazin-3(4H)-one (130 mg, Enantiomer A) in the same manner as described for Step 4 of EXAMPLE 3.

¹H NMR (DMSO-d₆): δ 1.66-2.08 (m, 8H), 3.24 (d, J=7.9 Hz, 1H), 3.51 (d, J=7.3 Hz, 1H), 3.70 (d, J=5.5 Hz, 1H), 3.92-4.04 (m, 2H), 4.04 (s, 3H), 4.67 (s, 2H), 5.23 (brs, 1H), 5.34 (brs, 1H), 5.77 (d, J=6.1 Hz, 1H), 7.10 (d, J=7.9 Hz, 1H), 7.25 (d, J=8.6 Hz, 1H), 7.41 (d, J=7.9 Hz, 1H), 8.29 (d, J=9.2 Hz, 1H), 8.72 (d, J=1.8 Hz, 1H), 8.98 (s, 2H), 11.26 (s, 1H).

MS (ESI⁺) m/z: 526 (MH⁺) (as free base).

HRMS (ESI⁺) for C₂₆H₂₉FN₅O₆ (MH⁺) (as free base): calcd, 526.21019. found, 526.20961.

Enantiomer B of 6-((1-(2-(3-fluoro-6-methoxy-1,5-naphthyridin-4-yl)-1,2-dihydroxyethyl)-2-oxabicyclo[2.2.2]octan-4-ylamino)methyl)-2H-pyrido[3,2-b][1,4]oxazin-3(4H)-one hydrochloride (77.7 mg) was prepared in the similar manner from 6-((1-(2-(3-fluoro-6-methoxy-1,5-naphthyridin-4-yl)-1,2-dihydroxyethyl)-2-oxabicyclo[2.2.2]octan-4-ylamino)methyl)-2H-pyrido[3,2-b][1,4]oxazin-3(4H)-one (125 mg, Enantiomer B).

¹H NMR (DMSO-d₆): δ 1.66-2.01 (m, 8H), 2.21-2.34 (m, 1H), 3.25 (d, J=6.7

Hz, 1H), 3.52 (d, J=7.9 Hz, 1H), 3.70 (d, J=5.5 Hz, 1H), 4.04 (s, 3H), 4.67 (s, 2H), 5.77 (d, J=6.1 Hz, 1H), 7.11 (d, J=7.9 Hz, 1H), 7.24 (d, J=9.2 Hz, 1H), 7.41 (d, J=7.9 Hz, 1H), 8.29 (d, J=9.2 Hz, 1H), 8.72 (d, J=1.8 Hz, 1H), 9.03 (s, 2H), 11.26 (s, 1H).

MS (ESI⁺) m/z: 526 (MH⁺) (as free base).

HRMS (ESI⁺) for C₂₆H₂₉FN₅O₆ (MH⁺) (as free base): calcd, 526.21019. found, 526.21096.

### Example 27

### 6-((1-(5-(3-Fluoro-6-methoxy-1,5-naphthyridin-4-yl)-2-oxo-1,3-dioxolan-4-yl)-2-oxabicyclo[2.2.2]octan-4-ylamino)methyl)-2H-pyrido[3,2-b][1,4]oxazin-3(4H)-one Hydrochloride (Enantiomer A)

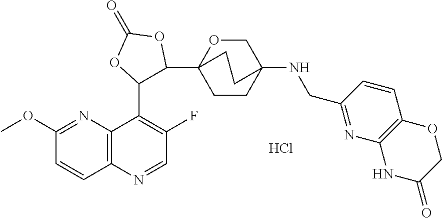 [SMILES: COc1ccc2ncc(F)c(C3OC(=O)OC3C34CCC(NCc5ccc6c(n5)NC(=O)CO6)(CC3)CO4)c2n1 ; Cl]

### Step 1

### tert-Butyl 1-(5-(3-Fluoro-6-methoxy-1,5-naphthyridin-4-yl)-2-oxo-1,3-dioxolan-4-yl)-2-oxabicyclo[2.2.2]octan-4-ylcarbamate (Enantiomer A)

To a solution of tert-butyl 1-(2-(3-fluoro-6-methoxy-1,5-naphthyridin-4-yl)-1,2-dihydroxyethyl)-2-oxabicyclo[2.2.2]octan-4-ylcarbamate (Example 26, Step 1, 270 mg) in dichloromethane (3.0 mL) was added triethylamine (146 μL) and triphosgene (176 mg) under cooling with ice, the mixture was stirred at the same temperature for 3 hours, and then concentrated in vacuo. Flash chromatography (silica, hexane:ethyl acetate=1:1) of the residue gave tert-butyl 1-(5-(3-fluoro-6-methoxy-1,5-naphthyridin-4-yl)-2-oxo-1,3-dioxolan-4-yl)-2-oxabicyclo[2.2.2]octan-4-ylcarbamate (222 mg, Enantiomer A).

¹H NMR (CDCl₃): δ 1.43 (s, 9H), 1.48-2.34 (m, 8H), 3.96-4.08 (m, 2H), 4.10 (s, 3H), 4.32 (brs, 1H), 4.73 (d, J=6.1 Hz, 1H), 6.39 (d, J=5.5 Hz, 1H), 7.13 (d, J=9.2 Hz, 1H), 8.23 (d, J=9.2 Hz, 1H), 8.71 (s, 1H).

MS (ESI⁺) m/z: 490 (MH⁺).

HRMS (ESI⁺) for C₂₄H₂₉FN₃O₇ (MH⁺): calcd, 490.19895. found, 490.19921.

Enantiomer B of tert-butyl 1-(5-(3-fluoro-6-methoxy-1,5-naphthyridin-4-yl)-2-oxo-1,3-dioxolan-4-yl)-2-oxabicyclo[2.2.2]octan-4-ylcarbamate (164 mg) was prepared in the similar manner from tert-butyl 1-(2-(3-fluoro-6-methoxy-1,5-naphthyridin-4-yl)-1,2-dihydroxyethyl)-2-oxabicyclo[2.2.2]octan-4-ylcarbamate (260 mg, Enantiomer B).

¹H NMR (CDCl₃): δ 1.43 (s, 9H), 1.58-1.99 (m, 6H), 2.08-2.35 (m, 2H), 3.96-4.10 (m, 2H), 4.10 (s, 3H), 4.32 (brs, 1H), 4.73 (d, J=6.1 Hz, 1H), 6.39 (d, J=5.5 Hz, 1H), 7.13 (d, J=9.2 Hz, 1H), 8.22 (d, J=9.2 Hz, 1H), 8.71 (s, 1H).

MS (ESI⁺) m/z: 490 (MH⁺).

HRMS (ESI⁺) for C₂₄H₂₉FN₃O₇ (MH⁺): calcd, 490.19895. found, 490.19983.

### Step 2

### 4-(4-Amino-2-oxabicyclo[2.2.2]octan-1-yl)-5-(3-fluoro-6-methoxy-1,5-naphthyridin-4-yl)-1,3-dioxolan-2-one (Enantiomer A)

The title compound 4-(4-amino-2-oxabicyclo[2.2.2]octan-1-yl)-5-(3-fluoro-6-methoxy-1,5-naphthyridin-4-yl)-1,3-dioxolan-2-one (84.5 mg) was prepared from tert-butyl 1-(5-(3-fluoro-6-methoxy-1,5-naphthyridin-4-yl)-2-oxo-1,3-dioxolan-4-yl)-2-oxabicyclo[2.2.2]octan-4-ylcarbamate (110 mg, Enantiomer A) in the same manner as described for Step 2 of EXAMPLE 1.

¹H NMR (CDCl₃): δ 1.35-2.33 (m, 8H), 3.64-3.75 (m, 2H), 4.10 (s, 3H), 4.73 (d, J=5.5 Hz, 1H), 6.40 (d, J=5.5 Hz, 1H), 7.13 (d, J=9.2 Hz, 1H), 8.22 (d, J=9.2 Hz, 1H), 8.71 (s, 1H).

MS (ESI⁺) m/z: 390 (MH⁺).

HRMS (ESI⁺) for C₁₉H₂₁FN₃O₅ (MH⁺): calcd, 390.14652. found, 390.14627.

Enantiomer B of 4-(4-amino-2-oxabicyclo[2.2.2]octan-1-yl)-5-(3-fluoro-6-methoxy-1,5-naphthyridin-4-yl)-1,3-dioxolan-2-one (119 mg) was prepared in the same manner from tert-butyl 1-(5-(3-fluoro-6-methoxy-1,5-naphthyridin-4-yl)-2-oxo-1,3-dioxolan-4-yl)-2-oxabicyclo[2.2.2]octan-4-ylcarbamate (150 mg, Enantiomer B).

¹H NMR (CDCl₃): δ 1.38-2.31 (m, 8H), 3.64-3.76 (m, 2H), 4.10 (s, 3H), 4.73 (d, J=6.1 Hz, 1H), 6.40 (d, J=6.1 Hz, 1H), 7.13 (d, J=9.2 Hz, 1H), 8.22 (d, J=9.2 Hz, 1H), 8.71 (s, 1H).

MS (ESI⁺) m/z: 390 (MH⁺).

HRMS (ESI⁺) for C₁₉H₂₁FN₃O₅ (MH⁺): calcd, 390.14652. found, 390.14601.

### Step 3

### 6-((1-(5-(3-Fluoro-6-methoxy-1,5-naphthyridin-4-yl)-2-oxo-1,3-dioxo lan-4-yl)-2-oxabicyclo[2.2.2]octan-4-ylamino)methyl)-2H-pyrido[3,2-b][1,4]oxazin-3(4H)-one (Enantiomer A)

The title compound 6-((1-(5-(3-fluoro-6-methoxy-1,5-naphthyridin-4-yl)-2-oxo-1,3-dioxolan-4-yl)-2-oxabicyclo[2.2.2]octan-4-ylamino)methyl)-2H-pyrido[3,2-b][1,4]oxazin-3(4H)-one (103 mg) was prepared from 4-(4-amino-2-oxabicyclo[2.2.2]octan-1-yl)-5-(3-fluoro-6-methoxy-1,5-naphthyridin-4-yl)-1,3-dioxolan-2-one (80.0 mg, Enantiomer A) and I (38.4 mg) in the same manner as described for Step 3 of EXAMPLE 1.

¹H NMR (DMSO-d₆): δ 1.44-1.81 (m, 6H), 1.98-2.10 (m, 2H), 3.62 (brs, 2H), 3.70 (s, 2H), 4.03 (s, 3H), 4.59 (s, 2H), 4.97 (d, J=5.5 Hz, 1H), 6.45 (d, J=5.5 Hz, 1H), 7.00 (d, J=7.9 Hz, 1H), 7.28 (d, J=7.9 Hz, 1H), 7.33 (d, J=9.2 Hz, 1H), 8.37 (d, J=9.2 Hz, 1H), 8.96 (s, 1H), 11.15 (s, 1H).

MS (ESI⁺) m/z: 552 (MH⁺).

HRMS (ESI⁺) for C₂₇H₂₇FN₅O₇ (MH⁺): calcd, 552.18945. found, 552.18987.

Enantiomer B of 6-((1-(5-(3-fluoro-6-methoxy-1,5-naphthyridin-4-yl)-2-oxo-1,3-dioxolan-4-yl)-2-oxabicyclo[2.2.2]octan-4-ylamino)methyl)-2H-pyrido[3,2-b][1,4]oxazin-3(4H)-one (159 mg) was prepared in the same manner from 4-(4-amino-2-oxabicyclo[2.2.2]octan-1-yl)-5-(3-fluoro-6-methoxy-1,5-naphthyridin-4-yl)-1,3-dioxolan-2-one (110 mg, Enantiomer B) and I (52.8 mg).

¹H NMR (DMSO-d₆): δ 1.42-1.81 (m, 6H), 1.98-2.10 (m, 2H), 3.63 (d, J=5.5 Hz, 1H), 3.70 (brs, 2H), 4.03 (s, 3H), 4.59 (s, 2H), 4.97 (d, J=5.5 Hz, 1H), 6.45 (d, J=5.5 Hz, 1H), 7.00 (d, J=7.9 Hz, 1H), 7.28 (d, J=7.9 Hz, 1H), 7.33 (d, J=9.2 Hz, 1H), 8.37 (d, J=9.2

Hz, 1H), 8.96 (s, 1H), 11.15 (s, 1H).

MS (ESI⁺) m/z: 552 (MH⁺).

HRMS (ESI⁺) for C₂₇H₂₇FN₅O7 (MH⁺): calcd, 552.18945. found, 552.18904.

### Step 4

### 6-((1-(5-(3-Fluoro-6-methoxy-1,5-naphthyridin-4-yl)-2-oxo-1,3-dioxo lan-4-yl)-2-oxabicyclo[2.2.2]octan-4-ylamino)methyl)-2H-pyrido[3,2-b][1,4]oxazin-3(4H)-one Hydrochloride (Enantiomer A)

The title compound 6-((1-(5-(3-fluoro-6-methoxy-1,5-naphthyridin-4-yl)-2-oxo-1,3-dioxo lan-4-yl)-2-oxabicyclo[2.2.2]octan-4-ylamino)methyl)-2H-pyrido[3,2-b][1,4]oxazin-3(4H)-one hydrochloride (71.8 mg) was prepared from 6-((1-(5-(3-fluoro-6-methoxy-1,5-naphthyridin-4-yl)-2-oxo-1,3-dioxo lan-4-yl)-2-oxabicyclo[2.2.2]octan-4-ylamino)methyl)-2H-pyrido[3,2-b][1,4]oxazin-3(4H)-one (90.0 mg, Enantiomer A) in the same manner as described for Step 4 of EXAMPLE 3.

¹H NMR (DMSO-d₆): δ 1.61-2.24 (m, 8H), 3.97-4.18 (m, 7H), 4.68 (s, 2H), 5.07 (d, J=4.9 Hz, 1H), 6.46 (d, J=5.5 Hz, 1H), 7.23 (d, J=7.9 Hz, 1H), 7.35 (d, J=8.6 Hz, 1H), 7.45 (d, J=7.9 Hz, 1H), 8.39 (d, J=9.2 Hz, 1H), 8.98 (s, 1H), 9.47 (s, 2H), 11.33 (s, 1H).

MS (ESI⁺) m/z: 552 (MH⁺) (as free base).

HRMS (ESI⁺) for C₂₇H₂₇FN₅O₇ (MH⁺) (as free base): calcd, 552.18945. found, 552.18865.

Enantiomer B of 6-((1-(5-(3-fluoro-6-methoxy-1,5-naphthyridin-4-yl)-2-oxo-1,3-dioxolan-4-yl)-2-oxabicyclo[2.2.2]octan-4-ylamino)methyl)-2H-pyrido[3,2-b][1,4]oxazin-3(4H)-one hydrochloride (66.3 mg) was prepared in the same manner from 6-((1-(5-(3-fluoro-6-methoxy-1,5-naphthyridin-4-yl)-2-oxo-1,3-dioxolan-4-yl)-2-oxabicyclo[2.2.2]octan-4-ylamino)methyl)-2H-pyrido[3,2-b][1,4]oxazin-3(4H)-one (75.0 mg, Enantiomer B).

¹H NMR (DMSO-d₆): δ 1.62-2.50 (m, 8H), 3.95-4.20 (m, 7H), 4.69 (s, 2H), 5.08 (d, J=5.5 Hz, 1H), 6.46 (d, J=5.5 Hz, 1H), 7.20 (d, J=7.9 Hz, 1H), 7.35 (d, J=9.2 Hz, 1H), 7.45 (d, J=7.9 Hz, 1H), 8.39 (d, J=9.2 Hz, 1H), 8.98 (s, 1H), 9.40 (s, 2H) 11.33 (s, 1H).

MS (ESI⁺) m/z: 552 (MH⁺) (as free base).

HRMS (ESI⁺) for C₂₇H₂₇FN₅O₇ (MH⁺) (as free base): calcd, 552.18945. found, 552.18940.

### Example 28

### 6-((1-(2-(3-Fluoro-6-morpholino-1,5-naphthyridin-4-yl)ethyl)-2-oxabicyclo[2.2.2]octan-4-ylamino)methyl)-2H-pyrido[3,2-b][1,4]oxazin-3(4H)-one

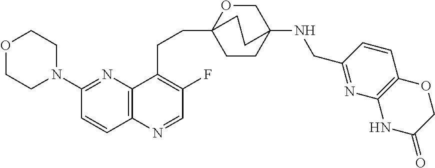 [SMILES: O=C1COc2ccc(CNC34CCC(CCc5c(F)cnc6ccc(N7CCOCC7)nc56)(CC3)OC4)nc2N1]

### Step 1

### 8-(2-(4-(tert-Butoxycarbonylamino)-2-oxabicyclo[2.2.2]octan-1-yl)ethyl)-7-fluoro-1,5-naphthyridin-2-yl Trifluoromethanesulfonate

To a solution of V (50.0 mg) in pyridine (1.2 mL) was added triflic anhydride (30 μL) at 0° C., the mixture was stirred at room temperature for 2 hours. After dilution of the mixture with dichloromethane, the mixture was washed with water. The organic extracts were dried over anhydrous sodium sulfate, filtered, and then concentrated in vacuo. Flash chromatography (silica, hexane:ethyl acetate=2:1) of the residue gave 8-(2-(4-(tert-butoxycarbonylamino)-2-oxabicyclo[2.2.2]octan-1-yl)ethyl)-7-fluoro-1,5-naphthyridin-2-yl trifluoromethanesulfonate (59.1 mg).

¹H NMR (CDCl₃): δ 1.43 (s, 9H), 1.70-2.21 (m, 10H), 3.19-3.23 (m, 2H), 3.94 (s, 2H), 4.28 (s, 1H), 7.38 (d, J=8.6 Hz, 1H), 8.55 (d, J=9.2 Hz, 1H), 8.85 (s, 1H).

MS (ESI⁺) m/z: 550 (MH⁺).

HRMS (ESI⁺) for C₂₃H₂₈F₄N₃O₆S (MH⁺): calcd, 550.16349. found, 550.16388.

### Step 2

### tert-Butyl 1-(2-(3-Fluoro-6-morpholino-1,5-naphthyridin-4-yl)ethyl)-2-oxabicyclo[2.2.2]octan-4-ylcarbamate

A mixture of 8-(2-(4-(tert-butoxycarbonylamino)-2-oxabicyclo[2.2.2]octan-1-yl)ethyl)-7-fluoro-1,5-naphthyridin-2-yltrifluoromethanesulfonate (90.0 mg) and morpholine (0.15 mL) in acetonitrile (1.6 mL) was stirred at 60° C. for 1 hour, and concentrated in vacuo. Flash chromatography (silica, hexane:ethyl acetate=1:1) of the residue gave tert-butyl 1-(2-(3-fluoro-6-morpholino-1,5-naphthyridin-4-yl)ethyl)-2-oxabicyclo[2.2.2]octan-4-ylcarbamate (80.0 mg).

¹H NMR (CDCl₃): δ 1.43 (s, 9H), 1.71-2.17 (m, 10H), 3.10-3.14 (m, 2H), 3.75 (t, J=4.9 Hz, 4H), 3.86 (t, J=4.9 Hz, 4H), 3.96 (s, 2H), 4.30 (s, 1H), 7.09 (d, J=9.8 Hz, 1H), 8.07 (d, J=9.2 Hz, 1H), 8.45 (s, 1H).

MS (ESI⁺) m/z: 487 (MH⁺).

HRMS (ESI⁺) for C₂₆H₃₆FN₄O₄ (MH⁺): calcd, 487.27206. found, 487.27225.

### Step 3

### 1-(2-(3-Fluoro-6-morpholino-1,5-naphthyridin-4-yl)ethyl)-2-oxabicyclo[2.2.2]octan-4-amine

The title compound 1-(2-(3-fluoro-6-morpholino-1,5-naphthyridin-4-yl)ethyl)-2-oxabicyclo[2.2.2]octan-4-amine (60.2 mg) was prepared from tert-butyl 1-(2-(3-fluoro-6-morpholino-1,5-naphthyridin-4-yl)ethyl)-2-oxabicyclo[2.2.2]octan-4-ylcarbamate (75.0 mg) in the same manner as described for Step 2 of EXAMPLE 1.

¹H NMR (CDCl₃): δ 1.64-2.05 (m, 12H), 3.11-3.15 (m, 2H), 3.65 (s, 2H), 3.75 (t, J=4.3 Hz, 4H), 3.86 (t, J=4.3 Hz, 4H), 7.09 (d, J=9.7 Hz, 1H), 8.07 (d, J=9.1 Hz, 1H), 8.46 (s, 1H).

MS (ESI⁺) m/z: 387 (MH⁺).

HRMS (ESI⁺) for C₂₁H₂₈FN₄O₂ (MH⁺): calcd, 387.21963. found, 387.21940.

### Step 4

### 6-((1-(2-(3-Fluoro-6-morpholino-1,5-naphthyridin-4-yl)ethyl)-2-oxabicyclo[2.2.2]octan-4-ylamino)methyl)-2H-pyrido[3,2-b][1,4]oxazin-3(4H)-one

The title compound 6-((1-(2-(3-fluoro-6-morpholino-1,5-naphthyridin-4-yl)ethyl)-2-oxabicyclo[2.2.2]octan-4-ylamino)methyl)-2H-pyrido[3,2-b][1,4]oxazin-3(4H)-one (62.2 mg) was prepared from 1-(2-(3-fluoro-6-morpholino-1,5-naphthyridin-4-yl)ethyl)-2-oxabicyclo[2.2.2]octan-4-amine (59.0 mg) and I (28.0 mg) in the same manner as described for Step 3 of EXAMPLE 1.

¹H NMR (DMSO-d₆): δ 1.53-1.96 (m, 11H), 3.00-3.04 (m, 2H), 3.57 (s, 2H), 3.62 (s, 2H), 3.72 (s, 8H), 4.59 (s, 2H), 7.01 (d, J=7.9 Hz, 1H), 7.28 (d, J=7.9 Hz, 1H), 7.41 (d, J=9.2 Hz, 1H), 8.06 (d, J=9.2 Hz, 1H), 8.51 (s, 1H), 11.16 (s, 1H).

MS (ESI⁺) m/z: 549 (MH⁺).

HRMS (ESI⁺) for C₂₉H₃₄FN₆O₄ (MH⁺): calcd, 549.26256. found, 549.26219.

### Example 29

### 6-((1-(2-(6-(Dimethylamino)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

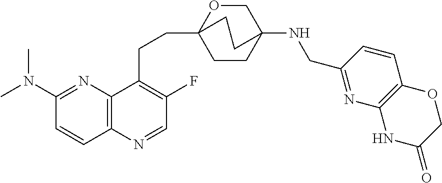 [SMILES: CN(C)c1ccc2ncc(F)c(CCC34CCC(NCc5ccc6c(n5)NC(=O)CO6)(CC3)CO4)c2n1]

### Step 1

### tert-Butyl 1-(2-(6-(Dimethylamino)-3-fluoro-1,5-naphthyridin-4-yl)ethyl)-2-oxabicyclo[2.2.2]octan-4-ylcarbamate

The title compound tert-butyl 1-(2-(6-(dimethylamino)-3-fluoro-1,5-naphthyridin-4-yl)ethyl)-2-oxabicyclo[2.2.2]octan-4-ylcarbamate (75.0 mg) was prepared from 8-(2-(4-(tert-butoxycarbonylamino)-2-oxabicyclo[2.2.2]octan-1-yl)ethyl)-7-fluoro-1,5-naphthyridin-2-yl trifluoromethanesulfonate (Example 28, Step 1, 90.0 mg) and dimethylamine (2.0 M in tetrahydrofuran, 0.8 mL) in the same manner as described for Step 2 of EXAMPLE 28.

¹H NMR (CDCl₃): δ 1.43 (s, 9H), 1.73-2.10 (m, 10H), 3.10-3.15 (m, 2H), 3.24 (s, 6H), 3.97 (s, 2H), 4.29 (s, 1H), 7.01 (d, J=9.8 Hz, 1H), 8.00 (d, J=9.2 Hz, 1H), 8.39 (s, 1H).

MS (ESI⁺) m/z: 445 (MH⁺).

HRMS (ESI⁺) for C₂₄H₃₄FN₄O₃ (MH⁺): calcd, 445.26149. found, 445.26057.

### Step 2

### 8-(2-(4-Amino-2-oxabicyclo[2.2.2]octan-1-yl)ethyl)-7-fluoro-N,N-dimethyl-1,5-naphthyridin-2-amine

The title compound 8-(2-(4-amino-2-oxabicyclo[2.2.2]octan-1-yl)ethyl)-7-fluoro-N,N-dimethyl-1,5-naphthyridin-2-amine (54.7 mg) was prepared from tert-butyl 1-(2-(6-(dimethylamino)-3-fluoro-1,5-naphthyridin-4-yl)ethyl)-2-oxabicyclo[2.2.2]octan-4-ylcarbamate (75.0 mg) in the same manner as described for Step 2 of EXAMPLE 1.

¹H NMR (CDCl₃): δ 1.50-2.05 (m, 12H), 3.11-3.16 (m, 2H), 3.25 (s, 6H), 3.65 (s, 2H), 7.02 (d, J=9.2 Hz, 1H), 8.01 (d, J=9.2 Hz, 1H), 8.39 (s, 1H).

MS (ESI⁺) m/z: 345 (MH⁺).

HRMS (ESI⁺) for C₁₉H₂₆FN₄O (MH⁺): calcd, 345.20906. found, 345.20944.

### Step 3

### 6-((1-(2-(6-(Dimethylamino)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

The title compound 6-((1-(2-(6-(dimethylamino)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one (40.5 mg) was prepared from 8-(2-(4-amino-2-oxabicyclo[2.2.2]octan-1-yl)ethyl)-7-fluoro-N,N-dimethyl-1,5-naphthyridin-2-amine (51.0 mg) and I (28.0 mg) in the same manner as described for Step 3 of EXAMPLE 1.

¹H NMR (DMSO-d₆): δ 1.55-1.93 (m, 11H), 3.00-3.04 (m, 2H), 3.19 (s, 6H), 3.58 (s, 2H), 3.62 (d, J=6.7 Hz, 2H), 4.59 (s, 2H), 7.00 (d, J=7.9 Hz, 1H), 7.25 (d, J=9.8 Hz, 1H), 7.27 (d, J=7.9 Hz, 1H), 8.00 (d, J=9.8 Hz, 1H), 8.44 (s, 1H), 11.16 (s, 1H).

MS (ESI⁺) m/z: 507 (MH⁺).

HRMS (ESI⁺) for C₂₇H₃₂FN₆O₃ (MH⁺): calcd, 507.25199. found, 507.25179.

### Example 30

### 6-((1-(2-(6-Amino-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

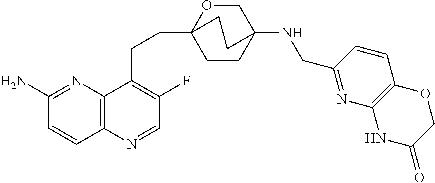 [SMILES: Nc1ccc2ncc(F)c(CCC34CCC(NCc5ccc6c(n5)NC(=O)CO6)(CC3)CO4)c2n1]

### Step 1

### tert-Butyl 1-(2-(3-Fluoro-6-(4-methoxybenzylamino)-1,5-naphthyridin-4-yl)ethyl)-2-oxabicyclo[2.2.2]octan-4-ylcarbamate

A mixture of 8-(2-(4-(tert-butoxycarbonylamino)-2-oxabicyclo[2.2.2]octan-1-yl)ethyl)-7-fluoro-1,5-naphthyridin-2-yltrifluoromethanesulfonate (Example 28, Step 1, 64.3 mg) and 4-methoxybenzylamine (0.1 mL) in acetonitrile (1.2 mL) was stirred at 60° C. for 5 hours and then concentrated in vacuo. Flash chromatography (silica, hexane:ethyl acetate=1:1) of the residue gave tert-butyl 1-(2-(3-fluoro-6-(4-methoxybenzylamino)-1,5-naphthyridin-4-yl)ethyl)-2-oxabicyclo[2.2.2]octan-4-ylcarbamate (63.0 mg).

¹H NMR (CDCl₃): δ 1.42 (s, 9H), 1.65-1.74 (m, 4H), 1.80-1.85 (m, 2H), 1.94-2.07 (m, 4H), 3.09-3.14 (m, 2H), 3.81 (s, 3H), 3.93 (s, 2H), 4.25 (brs, 1H), 4.69 (d, J=5.5 Hz, 2H), 5.09 (t, J=5.5 Hz, 1H), 6.75 (d, J=9.2 Hz, 1H), 6.88-6.90 (m, 2H), 7.31-7.34 (m, 2H), 7.95 (d, J=9.2 Hz, 1H), 8.41 (s, 1H).

MS (ESI⁺) m/z: 537 (MH⁺).

HRMS (ESI⁺) for C₃₀H₃₈FN₄O₄ (MH⁺): calcd, 537.28771. found, 537.28760.

### Step 2

### 8-(2-(4-Amino-2-oxabicyclo[2.2.2]octan-1-yl)ethyl)-7-fluoro-1,5-naphthyridin-2-amine

The title compound 8-(2-(4-amino-2-oxabicyclo[2.2.2]octan-1-yl)ethyl)-7-fluoro-1,5-naphthyridin-2-amine (36.8 mg) was prepared from tert-butyl 1-(2-(3-fluoro-6-(4-methoxybenzylamino)-1,5-naphthyridin-4-yl)ethyl)-2-oxabicyclo[2.2.2]octan-4-ylcarbamate (62.0 mg) in the same manner as described for Step 2 of EXAMPLE 32.

¹H NMR (DMSO-d₆): δ 1.51-1.70 (m, 10H), 1.78-1.82 (m, 2H), 2.95-2.99 (m, 2H), 3.45 (s, 2H), 6.73 (s, 2H), 6.91 (d, J=9.2 Hz, 1H), 7.88 (d, J=9.2 Hz, 1H), 8.39 (s, 1H).

MS (CI⁺) m/z: 317 (MH⁺).

HRMS (CI⁺) for C₁₇H₂₂FN₄O (MH⁺): calcd, 317.1778. found, 317.1808.

### Step 3

### 6-((1-(2-(6-Amino-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

The title compound 6-((1-(2-(6-amino-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one (31.8 mg) was prepared from 8-(2-(4-amino-2-oxabicyclo[2.2.2]octan-1-yl)ethyl)-7-fluoro-1,5-naphthyridin-2-amine (36.0 mg) and I (28.0 mg) in the same manner as described for Step 3 of EXAMPLE 1.

¹H NMR (DMSO-d₆): δ 1.51-1.76 (m, 8H), 1.76-1.98 (m, 3H), 2.96-3.00 (m, 2H), 3.57 (s, 2H), 3.63 (s, 2H), 4.59 (s, 2H), 6.74 (s, 2H), 6.91 (d, J=9.2 Hz, 1H), 7.01 (d, J=7.9 Hz, 1H), 7.28 (d, J=7.9 Hz, 1H), 7.89 (d, J=9.2 Hz, 1H), 8.39 (s, 1H), 11.16 (s, 1H).

MS (ESI⁺) m/z: 479 (MH⁺).

HRMS (ESI⁺) for C₂₅H₂₈FN₆O₃ (MH⁺): calcd, 479.22069. found, 479.22037.

### Example 31

### 7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-2-carbonitrile

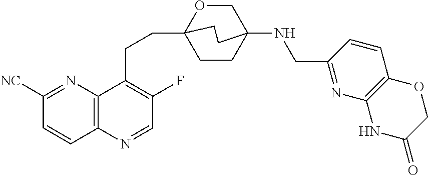 [SMILES: N#Cc1ccc2ncc(F)c(CCC34CCC(NCc5ccc6c(n5)NC(=O)CO6)(CC3)CO4)c2n1]

### Step 1

### tert-Butyl 1-(2-(6-Cyano-3-fluoro-1,5-naphthyridin-4-yl)ethyl)-2-oxabicyclo[2.2.2]octan-4-ylcarbamate

A mixture of 8-(2-(4-(tert-butoxycarbonylamino)-2-oxabicyclo[2.2.2]octan-1-yl)ethyl)-7-fluoro-1,5-naphthyridin-2-yltrifluoromethanesulfonate (Example 28, Step 1, 20.0 mg), zinc(II) cyanide (4.40 mg) and tetrakis(triphenylphosphine)palladium (12.7 mg) in N-methyl-2-pyrrolidone (0.3 mL) was heated at 80° C. for 1 hour and concentrated in vacuo. After dilution of the residue with dichloromethane, the mixture was washed with water and brine. The organic extracts were dried over anhydrous sodium sulfate, filtered, and then concentrated in vacuo. Flash chromatography (silica, hexane:ethyl acetate=2:1) of the residue gave tert-butyl 1-(2-(6-cyano-3-fluoro-1,5-naphthyridin-4-yl)ethyl)-2-oxabicyclo[2.2.2]octan-4-ylcarbamate (16.0 mg).

¹H NMR (CDCl₃): δ 1.43 (s, 9H), 1.75-2.12 (m, 10H), 3.28-3.32 (m, 2H), 3.93 (s, 2H), 4.29 (s, 1H), 7.89 (d, J=8.6 Hz, 1H), 8.53 (d, J=8.6 Hz, 1H), 8.92 (s, 1H).

MS (ESI⁺) m/z: 427 (MH⁺).

HRMS (ESI⁺) for C₂₃H₂₈FN₄O₃ (MH⁺): calcd, 427.21454. found, 427.21492.

### Step 2

### 8-(2-(4-Amino-2-oxabicyclo[2.2.2]octan-1-yl)ethyl)-7-fluoro-1,5-naphthyridine-2-carbonitrile

The title compound 8-(2-(4-amino-2-oxabicyclo[2.2.2]octan-1-yl)ethyl)-7-fluoro-1,5-naphthyridine-2-carbonitrile (57.5 mg) was prepared from tert-butyl 1-(2-(6-cyano-3-fluoro-1,5-naphthyridin-4-yl)ethyl)-2-oxabicyclo[2.2.2]octan-4-ylcarbamate (78.4 mg) in the same manner as described for Step 2 of EXAMPLE 1.

¹H NMR (CDCl₃): δ 1.67-2.05 (m, 12H), 3.29-3.34 (m, 2H), 3.62 (s, 2H), 7.89 (d, J=8.6 Hz, 1H), 8.52 (d, J=9.2 Hz, 1H), 8.92 (s, 1H).

MS (ESI⁺) m/z: 327 (MH⁺).

HRMS (ESI⁺) for C₁₈H₂₀FN₄O (MH⁺): calcd, 327.16211. found, 327.16209.

### Step 3

### 7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-2-carbonitrile

The title compound 7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-2-carbonitrile (60.2 mg) was prepared from 8-(2-(4-amino-2-oxabicyclo[2.2.2]octan-1-yl)ethyl)-7-fluoro-1,5-naphthyridine-2-carbonitrile (54.8 mg) and I (33.0 mg) in the same manner as described for Step 3 of EXAMPLE 1.

¹H NMR (DMSO-d₆): δ 1.62-1.88 (m, 11H), 3.18-3.21 (m, 2H), 3.55 (s, 2H), 3.62 (s, 2H), 4.59 (s, 2H), 7.00 (d, J=7.9 Hz, 1H), 7.27 (d, J=7.9 Hz, 1H), 8.28 (d, J=8.6 Hz, 1H), 8.71 (d, J=8.6 Hz, 1H), 9.16 (s, 1H), 11.15 (s, 1H).

MS (ESI⁺) m/z: 489 (MH⁺).

HRMS (ESI⁺) for C₂₆H₂₆FN₆O₃ (MH⁺): calcd, 489.20504. found, 489.20558.

### Example 32

### Ethyl 2-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)acetate

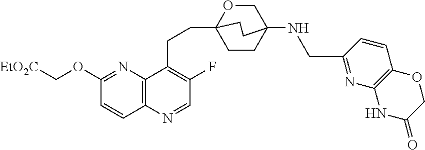 [SMILES: CCOC(=O)COc1ccc2ncc(F)c(CCC34CCC(NCc5ccc6c(n5)NC(=O)CO6)(CC3)CO4)c2n1]

### Step 1

### Ethyl 2-(8-(2-(4-(tert-Butoxycarbonylamino)-2-oxabicyclo[2.2.2]octan-1-yl)ethyl)-7-fluoro-1,5-naphthyridin-2-yloxy)acetate

A mixture of V (50.0 mg) and potassium carbonate (24.8 mg) in N,N-dimethylformamide (2.4 mL) was stirred at room temperature for 1 hour. The mixture was added ethyl bromoacetate (22.0 mg) under cooling with ice, the mixture was stirred at room temperature for 4 hours and then concentrated in vacuo. After dilution of the residue with ethyl acetate, the mixture was washed with 1 M hydrochloric acid and saturated sodium hydrogencarbonate solution. The organic extracts were dried over anhydrous sodium sulfate, filtered, and then concentrated in vacuo. Flash chromatography (silica, chloroform:methanol=30:1) of the residue gave ethyl 2-(8-(2-(4-(tert-butoxycarbonylamino)-2-oxabicyclo[2.2.2]octan-1-yl)ethyl)-7-fluoro-1,5-naphthyridin-2-yloxy)acetate (60.0 mg).

¹H NMR (DMSO-d₆): δ 1.18 (t, J=6.7 Hz, 3H), 1.36 (s, 9H), 1.49-2.04 (m, 10H), 2.95-3.05 (m, 2H), 3.78 (s, 2H), 4.13 (q, J=7.1 Hz, 2H), 5.11 (s, 2H), 6.58 (s, 1H), 7.34 (d, J=9.2 Hz, 1H), 8.33 (d, J=9.2 Hz, 1H), 8.77 (s, 1H).

MS (ESI⁺) m/z: 504 (MH⁺).

HRMS (ESI⁺) for C₂₆H₃₅FN₃O₆ (MH⁺): calcd, 504.25099. found, 504.25013.

### Step 2

### Ethyl 2-(8-(2-(4-Amino-2-oxabicyclo[2.2.2]octan-1-yl)ethyl)-7-fluoro-1,5-naphthyridin-2-yloxy)acetate

To a solution of ethyl 2-(8-(2-(4-(tert-butoxycarbonylamino)-2-oxabicyclo[2.2.2]octan-1-yl)ethyl)-7-fluoro-1,5-naphthyridin-2-yloxy)acetate (59.0 mg) in dichloromethane (0.5 mL) was added trifluoroacetic acid (0.5 mL) under cooling with ice, the mixture was stirred at the same temperature for 2 hours and then concentrated in vacuo. A solution of the residue in methanol was charged into PoraPak Rxn CX column. The column was eluted with methanol and then with methanolconcentrated ammonium hydroxide (95:5) to give ethyl 2-(8-(2-(4-amino-2-oxabicyclo[2.2.2]octan-1-yl)ethyl)-7-fluoro-1,5-naphthyridin-2-yloxy)acetate (43.6 mg).

¹H NMR (CDCl₃): δ 1.24 (t, J=7.0 Hz, 3H), 1.45-2.06 (m, 12H), 3.07-3.16 (m, 2H), 3.65 (s, 2H), 4.25 (q, J=7.1 Hz, 2H), 5.07 (d, J=3.1 Hz, 2H), 7.20 (d, J=9.2 Hz, 1H), 8.24 (d, J=9.2 Hz, 1H), 8.62 (s, 1H).

MS (ESI⁺) m/z: 404 (MH⁺).

HRMS (ESI⁺) for C₂₁H₂₇FN₃O₄ (MH⁺): calcd, 404.19856. found, 404.19873.

### Step 3

### Ethyl 2-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)acetate

The title compound ethyl 2-(7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)acetate (38.7 mg) was prepared from ethyl 2-(8-(2-(4-amino-2-oxabicyclo[2.2.2]octan-1-yl)ethyl)-7-fluoro-1,5-naphthyridin-2-yloxy)acetate (38.0 mg) and I (17.6 mg) in the same manner as described for Step 3 of EXAMPLE 1.

¹H NMR (DMSO-d₆): δ 1.17 (t, J=7.3 Hz, 3H), 1.49-1.85 (m, 10H), 2.90-3.07 (m, 2H), 3.58 (s, 2H), 3.63 (s, 2H), 3.68 (s, 1H), 4.13 (q, J=7.3 Hz, 2H), 4.59 (s, 2H), 5.11 (d, J=3.7 Hz, 2H), 7.01 (d, J=7.9 Hz, 1H), 7.28 (d, J=7.9 Hz, 1H), 7.34 (d, J=8.6 Hz, 1H), 8.33 (d, J=9.2 Hz, 1H), 8.77 (s, 1H), 11.15 (s, 1H).

MS (ESI⁺) m/z: 566 (MH⁺).

HRMS (ESI⁺) for C₂₉H₃₃FN₅O₆ (MH⁺): calcd, 566.24149. found, 566.24173.

### Example 33

### 6-((1-(2-(3-Fluoro-6-(2-methoxyethoxy)-1,5-naphthyridin-4-yl)ethyl)-2-oxabicyclo[2.2.2]octan-4-ylamino)methyl)-2H-pyrido[3,2-b][1,4]oxazin-3(4H)-one

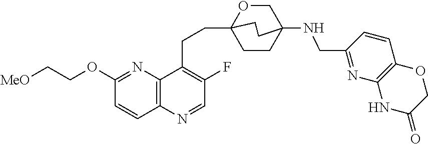 [SMILES: COCCOc1ccc2ncc(F)c(CCC34CCC(NCc5ccc6c(n5)NC(=O)CO6)(CC3)CO4)c2n1]

### Step 1

### tert-Butyl 1-(2-(3-Fluoro-6-(2-methoxyethoxy)-1,5-naphthyridin-4-yl)ethyl)-2-oxabicyclo[2.2.2]octan-4-ylcarbamate

The title compound tert-butyl 1-(2-(3-fluoro-6-(2-methoxyethoxy)-1,5-naphthyridin-4-yl)ethyl)-2-oxabicyclo[2.2.2]octan-4-ylcarbamate (49.1 mg) was prepared from V (50.0 mg) and 1-bromo-2-methoxyethane (18.3 mg) in the same manner as described for Step 1 of EXAMPLE 32.

¹H NMR (DMSO-d₆): δ 1.36 (s, 9H), 1.57-1.65 (m, 2H), 1.53-2.04 (m, 8H), 3.00-3.13 (m, 2H), 3.31 (s, 1H), 3.71-3.77 (m, 2H), 3.77 (s, 2H), 4.58 (t, J=4.9 Hz, 2H), 6.59 (s, 1H), 7.23 (d, J=9.2 Hz, 1H), 8.26 (d, J=9.2 Hz, 1H), 8.74 (s, 1H).

MS (ESI⁺) m/z: 476 (MH⁺).

HRMS (ESI⁺) for C₂₅H₃₅FN₃O₅ (MH⁺): calcd, 476.25607. found, 476.25577.

### Step 2

### 1-(2-(3-Fluoro-6-(2-methoxyethoxy)-1,5-naphthyridin-4-yl)ethyl)-2-oxabicyclo[2.2.2]octan-4-amine

The title compound 1-(2-(3-fluoro-6-(2-methoxyethoxy)-1,5-naphthyridin-4-yl)ethyl)-2-oxabicyclo[2.2.2]octan-4-amine (36.7 mg) was prepared from tert-butyl 1-(2-(3-fluoro-6-(2-methoxyethoxy)-1,5-naphthyridin-4-yl)ethyl)-2-oxabicyclo[2.2.2]octan-4-ylcarbamate (47.0 mg) in the same manner as described for Step 2 of EXAMPLE 32.

¹H NMR (DMSO-d₆): δ 1.42-1.88 (m, 10H), 3.12-3.02 (m, 2H), 3.31 (s, 3H), 3.45 (s, 2H), 3.74 (t, J=4.9 Hz, 2H), 4.58 (t, J=4.9 Hz, 2H), 7.23 (d, J=9.2 Hz, 1H), 8.26 (d, J=9.2 Hz, 1H), 8.74 (s, 1H).

MS (ESI⁺) m/z: 376 (MH⁺).

HRMS (ESI⁺) for C₂₀H₂₇FN₃O₃ (MH⁺): calcd, 376.20364. found, 376.20448.

### Step 3

### 6-((1-(2-(3-Fluoro-6-(2-methoxyethoxy)-1,5-naphthyridin-4-yl)ethyl)-2-oxabicyclo[2.2.2]octan-4-ylamino)methyl)-2H-pyrido[3,2-b][1,4]oxazin-3(4H)-one

The title compound 6-((1-(2-(3-fluoro-6-(2-methoxyethoxy)-1,5-naphthyridin-4-yl)ethyl)-2-oxabicyclo[2.2.2]octan-4-ylamino)methyl)-2H-pyrido[3,2-b][1,4]oxazin-3(4H)-one (40.0 mg) was prepared from 1-(2-(3-fluoro-6-(2-methoxyethoxy)-1,5-naphthyridin-4-yl)ethyl)-2-oxabicyclo[2.2.2]octan-4-amine (35.0 mg) and I (17.4 mg) in the same manner as described for Step 3 of EXAMPLE 1.

¹H NMR (DMSO-d₆): δ 1.57-1.93 (m, 10H), 3.04-3.14 (m, 2H), 3.31 (s, 3H), 3.58 (s, 2H), 3.63 (d, J=5.5 Hz, 2H), 3.74 (t, J=4.6 Hz, 2H), 4.57-4.59 (m, 4H), 7.01 (d, J=8.6 Hz, 1H), 7.23 (d, J=8.6 Hz, 1H), 7.28 (d, J=7.9 Hz, 1H), 8.26 (d, J=9.2 Hz, 1H), 8.74 (s, 1H), 11.14 (s, 1H).

MS (ESI⁺) m/z: 538 (MH⁺).

HRMS (ESI⁺) for C₂₈H₃₃FN₅O₅ (MH⁺): calcd, 538.24657. found, 538.24628.

### Example 34

### 6-((1-(2-(3-Fluoro-6-(2-hydroxyethoxy)-1,5-naphthyridin-4-yl)ethyl)-2-oxabicyclo[2.2.2]octan-4-ylamino)methyl)-2H-pyrido[3,2-b][1,4]oxazin-3(4H)-one Hydrochloride

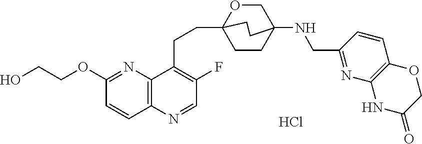 [SMILES: O=C1COc2ccc(CNC34CCC(CCc5c(F)cnc6ccc(OCCO)nc56)(CC3)OC4)nc2N1 ; Cl]

### Step 1

### tert-Butyl 1-(2-(3-Fluoro-6-(2-(tetrahydro-2H-pyran-2-yloxy)ethoxy)-1,5-naphthyridin-4-yl)ethyl)-2-oxabicyclo[2.2.2]octan-4-ylcarbamate

The title compound tert-butyl 1-(2-(3-fluoro-6-(2-(tetrahydro-2H-pyran-2-yloxy)ethoxy)-1,5-naphthyridin-4-yl)ethyl)-2-oxabicyclo[2.2.2]octan-4-ylcarbamate (51.5 mg) was prepared from V (50.0 mg) and 2-(2-bromoethoxy)tetrahydro-2H-pyran (27.5 mg) in the same manner as described for Step 1 of EXAMPLE 32.

¹H NMR (CDCl₃): δ 1.24-2.10 (m, 25H), 3.09-3.23 (m, 2H), 3.44-3.61 (m, 1H), 3.82-3.93 (m, 2H), 3.96 (s, 2H), 4.02-4.16 (m, 1H), 4.29 (s, 1H), 4.63-4.73 (m, 3H), 7.10 (d, J=9.2 Hz, 1H), 8.16 (d, J=9.2 Hz, 1H), 8.59 (s, 1H).

MS (ESI⁺) m/z: 546 (MH⁺).

HRMS (ESI⁺) for C₂₉H₄₁FN₃O₆ (MH⁺): calcd, 546.29794. found, 546.29739.

### Step 2

### 2-(8-(2-(4-Amino-2-oxabicyclo[2.2.2]octan-1-yl)ethyl)-7-fluoro-1,5-naphthyridin-2-yloxy)ethanol

The title compound 2-(8-(2-(4-amino-2-oxabicyclo[2.2.2]octan-1-yl)ethyl)-7-fluoro-1,5-naphthyridin-2-yloxy)ethanol (30.5 mg) was prepared from tert-butyl 1-(2-(3-fluoro-6-(2-(tetrahydro-2H-pyran-2-yloxy)ethoxy)-1,5-naphthyridin-4-yl)ethyl)-2-oxabicyclo[2.2.2]octan-4-ylcarbamate (48.0 mg) in the same manner as described for Step 2 of EXAMPLE 32.

¹H NMR (DMSO-d₆): δ 1.48-1.90 (m, 12H), 3.12-3.01 (m, 2H), 3.46 (s, 2H), 3.80 (q, J=4.9 Hz, 2H), 4.47 (t, J=4.9 Hz, 2H), 4.88 (t, J=5.5 Hz, 1H), 7.21 (d, J=9.2 Hz, 1H), 8.25 (d, J=9.2 Hz, 1H), 8.73 (s, 1H).

MS (ESI⁺) m/z: 362 (MH⁺).

HRMS (ESI⁺) for C₁₉H₂₅FN₃O₃ (MH⁺): calcd, 362.18843. found, 362.18799.

### Step 3

### 6-((1-(2-(3-Fluoro-6-(2-hydroxyethoxy)-1,5-naphthyridin-4-yl)ethyl)-2-oxabicyclo[2.2.2]octan-4-ylamino)methyl)-2H-pyrido[3,2-b][1,4]oxazin-3(4H)-one

The title compound 6-((1-(2-(3-fluoro-6-(2-hydroxyethoxy)-1,5-naphthyridin-4-yl)ethyl)-2-oxabicyclo[2.2.2]octan-4-ylamino)methyl)-2H-pyrido[3,2-b][1,4]oxazin-3(4H)-one (27.0 mg) was prepared from 2-(8-(2-(4-amino-2-oxabicyclo[2.2.2]octan-1-yl)ethyl)-7-fluoro-1,5-naphthyridin-2-yloxy)ethanol (28.0 mg) and I (14.5 mg) in the same manner as described for Step 3 of EXAMPLE 1.

¹H NMR (DMSO-d₆): δ 1.55-2.00 (m, 11H), 3.03-3.14 (m, 2H), 3.58 (s, 2H), 3.62 (s, 2H), 3.80 (q, J=4.9 Hz, 2H), 4.47 (d, J=4.9 Hz, 2H), 4.59 (s, 2H), 4.88 (t, J=5.5 Hz, 1H), 7.01 (d, J=7.9 Hz, 1H), 7.21 (d, J=9.2 Hz, 1H), 7.28 (d, J=7.9 Hz, 1H), 8.26 (d, J=9.2 Hz, 1H), 8.73 (s, 1H), 11.15 (s, 1H).

MS (ESI⁺) m/z: 524 (MH⁺).

HRMS (ESI⁺) for C₂₇H₃₁FN₅O₅ (MH⁺): calcd, 524.23092. found, 524.23000.

### Step 4

### 6-((1-(2-(3-Fluoro-6-(2-hydroxyethoxy)-1,5-naphthyridin-4-yl)ethyl)-2-oxabicyclo[2.2.2]octan-4-ylamino)methyl)-2H-pyrido[3,2-b][1,4]oxazin-3(4H)-one Hydrochloride

The title compound 6-((1-(2-(3-fluoro-6-(2-hydroxyethoxy)-1,5-naphthyridin-4-yl)ethyl)-2-oxabicyclo[2.2.2]octan-4-ylamino)methyl)-2H-pyrido[3,2-b][1,4]oxazin-3(4H)-one hydrochloride (397 mg) was prepared from 6-((1-(2-(3-fluoro-6-(2-hydroxyethoxy)-1,5-naphthyridin-4-yl)ethyl)-2-oxabicyclo[2.2.2]octan-4-ylamino)methyl)-2H-pyrido[3,2-b][1,4]oxazin-3(4H)-one (380 mg) in the same manner as described for Step 4 of EXAMPLE 3.

¹H NMR (DMSO-d₆): δ 1.64-1.74 (m, 2H), 1.77-1.90 (m, 2H), 1.92-2.10 (m, 6H), 3.05-3.15 (m, 2H), 3.77-3.85 (m, 2H), 3.92 (s, 2H), 4.04-4.14 (m, 2H), 4.48 (t, J=5.5 Hz, 2H), 4.69 (s, 2H), 4.93 (brs, 1H), 7.22 (d, J=8.6 Hz, 1H), 7.23 (d, J=9.2 Hz, 1H), 7.45 (d, J=8.6 Hz, 1H), 8.27 (d, J=9.2 Hz, 1H), 8.75 (s, 1H), 9.30 (brs, 2H), 11.33 (s, 1H).

MS (ESI⁺) m/z: 524 (MH⁺).

HRMS (ESI⁺) for C₂₇H₃₁FN₅O₅ (MH⁺): calcd, 524.23092. found, 524.23093.

### Example 35

### 6-((1-(2-(3-Fluoro-6-(3-hydroxypropoxy)-1,5-naphthyridin-4-yl)ethyl)-2-oxabicyclo[2.2.2]octan-4-ylamino)methyl)-2H-pyrido[3,2-b][1,4]oxazin-3(4H)-one Hydrochloride

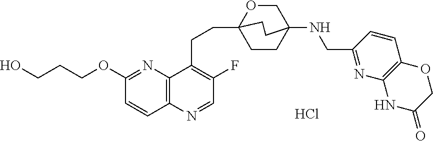 [SMILES: O=C1COc2ccc(CNC34CCC(CCc5c(F)cnc6ccc(OCCCO)nc56)(CC3)OC4)nc2N1 ; Cl]

### Step 1

### tert-Butyl 1-(2-(3-Fluoro-6-(3-(tetrahydro-2H-pyran-2-yloxy)propoxy)-1,5-naphthyridin-4-yl)ethyl)-2-oxabicyclo[2.2.2]octan-4-ylcarbamate

The title compound tert-butyl 1-(2-(3-fluoro-6-(3-(tetrahydro-2H-pyran-2-yloxy)propoxy)-1,5-naphthyridin-4-yl)ethyl)-2-oxabicyclo[2.2.2]octan-4-ylcarbamate (116 mg) was prepared from V (100 mg) and 2-(3-bromopropoxy)tetrahydro-2H-pyran (58.8 mg) in the same manner as described for Step 1 of EXAMPLE 32.

¹H NMR (CDCl₃): δ 1.43 (s, 9H), 1.52-2.21 (m, 18H), 3.11-3.23 (m, 2H), 3.47-3.56 (m, 1H), 3.56-3.65 (m, 1H), 3.84-3.91 (m, 1H), 3.91-4.01 (m, 3H), 4.28 (s, 1H), 4.58-4.64 (m, 3H), 7.04 (d, J=9.2 Hz, 1H), 8.15 (d, J=9.2 Hz, 1H), 8.58 (s, 1H).

MS (ESI⁺) m/z: 560 (MH⁺).

HRMS (ESI⁺) for C₃₀H₄₃FN₃O₆ (MH⁺): calcd, 560.31359. found, 560.31282.

### Step 2

### 3-(8-(2-(4-Amino-2-oxabicyclo[2.2.2]octan-1-yl)ethyl)-7-fluoro-1,5-naphthyridin-2-yloxy)propan-1-ol

The title compound 3-(8-(2-(4-amino-2-oxabicyclo[2.2.2]octan-1-yl)ethyl)-7-fluoro-1,5-naphthyridin-2-yloxy)propan-1-ol (70.1 mg) was prepared from tert-butyl 1-(2-(3-fluoro-6-(3-(tetrahydro-2H-pyran-2-yloxy)propoxy)-1,5-naphthyridin-4-yl)ethyl)-2-oxabicyclo[2.2.2]octan-4-ylcarbamate (110 mg) in the same manner as described for Step 2 of EXAMPLE 32.

¹H NMR (CDCl₃): δ 1.48-2.00 (m, 14H), 3.08 (t, J=8.3 Hz, 2H), 3.47 (s, 2H), 3.58 (t, J=5.2 Hz, 2H), 4.47-4.61 (m, 3H), 7.19 (d, J=9.2 Hz, 1H), 8.24 (d, J=9.2 Hz, 1H), 8.72 (s, 1H).

MS (ESI⁺) m/z: 376 (MH⁺).

HRMS (ESI⁺) for C₂₀H₂₇FN₃O₃ (MH⁺): calcd, 376.20364. found, 376.20417.

### Step 3

### 6-((1-(2-(3-Fluoro-6-(3-hydroxypropoxy)-1,5-naphthyridin-4-yl)ethyl)-2-oxabicyclo[2.2.2]octan-4-ylamino)methyl)-2H-pyrido[3,2-b][1,4]oxazin-3(4H)-one

The title compound 6-((1-(2-(3-fluoro-6-(3-hydroxypropoxy)-1,5-naphthyridin-4-yl)ethyl)-2-oxabicyclo[2.2.2]octan-4-ylamino)methyl)-2H-pyrido[3,2-b][1,4]oxazin-3(4H)-one (69.4 mg) was prepared from 3-(8-(2-(4-amino-2-oxabicyclo[2.2.2]octan-1-yl)ethyl)-7-fluoro-1,5-naphthyridin-2-yloxy)propan-1-ol (65.0 mg) and I (32.4 mg) in the same manner as described for Step 3 of EXAMPLE 1.

¹H NMR (DMSO-d₆): δ 1.58-2.00 (m, 12H), 3.05-3.14 (m, 2H), 3.34-3.67 (m, 6H), 4.50-4.58 (m, 3H), 4.59 (s, 2H), 7.01 (d, J=7.9 Hz, 1H), 7.19 (d, J=9.2 Hz, 1H), 7.28 (d, J=7.9 Hz, 1H), 8.24 (d, J=8.6 Hz, 1H), 8.73 (s, 1H), 11.15 (s, 1H).

MS (ESI⁺) m/z: 538 (MH⁺).

HRMS (ESI⁺) for C₂₈H₃₃FN₅O₅ (MH⁺): calcd, 538.24657. found, 538.24699.

### Step 4

### 6-((1-(2-(3-Fluoro-6-(3-hydroxypropoxy)-1,5-naphthyridin-4-yl)ethyl)-2-oxabicyclo[2.2.2]octan-4-ylamino)methyl)-2H-pyrido[3,2-b][1,4]oxazin-3(4H)-one Hydrochloride

The title compound 6-((1-(2-(3-fluoro-6-(3-hydroxypropoxy)-1,5-naphthyridin-4-yl)ethyl)-2-oxabicyclo[2.2.2]octan-4-ylamino)methyl)-2H-pyrido[3,2-b][1,4]oxazin-3(4H)-one hydrochloride (253 mg) was prepared from 6-((1-(2-(3-fluoro-6-(3-hydroxypropoxy)-1,5-naphthyridin-4-yl)ethyl)-2-oxabicyclo[2.2.2]octan-4-ylamino)methyl)-2H-pyrido[3,2-b][1,4]oxazin-3(4H)-one (275 mg) in the same manner as described for Step 4 of EXAMPLE 3.

¹H NMR (DMSO-d₆): δ 1.65-1.75 (m, 2H), 1.78-1.88 (m, 2H), 1.91-2.10 (m, 8H), 3.06-3.15 (m, 2H), 3.59 (t, J=6.1 Hz, 2H), 3.92 (s, 2H), 4.10 (t, J=6.1 Hz, 1H), 4.58 (t, J=6.1 Hz, 1H), 4.60 (br, 1H), 4.69 (s, 2H), 7.21 (d, J=9.2 Hz, 1H), 7.23 (d, J=7.9 Hz, 1H), 7.45 (d, J=8.6 Hz, 1H), 8.26 (d, J=8.6 Hz, 1H), 8.75 (s, 1H), 9.32 (br, 2H), 11.33 (s, 1H).

MS (ESI⁺) m/z: 538 (MH⁺).

HRMS (ESI⁺) for C₂₈H₃₃FN₅O₅ (MH⁺): calcd, 538.24657. found, 538.24600.

### Example 36

### 6-((1-(2-(3-Fluoro-6-(4-hydroxybutoxy)-1,5-naphthyridin-4-yl)ethyl)-2-oxabicyclo[2.2.2]octan-4-ylamino)methyl)-2H-pyrido[3,2-b][1,4]oxazin-3(4H)-one

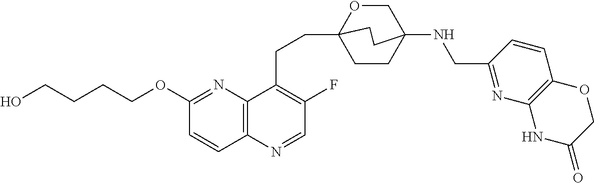 [SMILES: O=C1COc2ccc(CNC34CCC(CCc5c(F)cnc6ccc(OCCCCO)nc56)(CC3)OC4)nc2N1]

### Step 1

### tert-Butyl 1-(2-(3-Fluoro-6-(4-(tetrahydro-2H-pyran-2-yloxy)butoxy)-1,5-naphthyridin-4-yl)ethyl)-2-oxabicyclo[2.2.2]octan-4-ylcarbamate

The title compound tert-butyl 1-(2-(3-fluoro-6-(4-(tetrahydro-2H-pyran-2-yloxy)butoxy)-1,5-naphthyridin-4-yl)ethyl)-2-oxabicyclo[2.2.2]octan-4-ylcarbamate (103 mg) was prepared from V (80.0 mg) and 2-(4-bromobutoxy)tetrahydro-2H-pyran (49.9 mg) in the same manner as described for Step 1 of EXAMPLE 32.

¹H NMR (CDCl₃): δ 1.43 (s, 9H), 1.70-2.13 (m, 20H), 3.16 (t, J=8.3 Hz, 2H), 3.46-3.57 (m, 2H), 3.80-3.92 (m, 2H), 3.96 (s, 2H), 4.29 (s, 1H), 4.51 (t, J=6.4 Hz, 2H), 4.61 (t, J=3.7 Hz, 1H), 7.03 (d, J=9.2 Hz, 1H), 8.14 (d, J=9.2 Hz, 1H), 8.58 (s, 1H).

MS (ESI⁺) m/z: 574 (MH⁺).

HRMS (ESI⁺) for C₃₁H₄₅FN₃O₆ (MH⁺): calcd, 574.32924. found, 574.32842.

### Step 2

### 4-(8-(2-(4-Amino-2-oxabicyclo[2.2.2]octan-1-yl)ethyl)-7-fluoro-1,5-naphthyridin-2-yloxy)butan-1-ol

The title compound 4-(8-(2-(4-amino-2-oxabicyclo[2.2.2]octan-1-yl)ethyl)-7-fluoro-1,5-naphthyridin-2-yloxy)butan-1-ol (71.0 mg) was prepared from tert-butyl 1-(2-(3-fluoro-6-(4-(tetrahydro-2H-pyran-2-yloxy)butoxy)-1,5-naphthyridin-4-yl)ethyl)-2-oxabicyclo[2.2.2]octan-4-ylcarbamate (98.3 mg) in the same manner as described for Step 2 of EXAMPLE 32.

¹H NMR (CDCl₃): δ 1.53-2.09 (m, 17H), 3.13-3.24 (m, 2H), 3.68 (s, 2H), 3.75 (d, J=6.4 Hz, 2H), 4.55 (d, J=6.7 Hz, 2H), 7.03 (d, J=9.2 Hz, 1H), 8.15 (d, J=8.6 Hz, 1H), 8.58 (s, 1H).

MS (ESI⁺) m/z: 390 (MH⁺).

HRMS (ESI⁺) for C₂₁H₂₉FN₃O₃ (MH⁺): calcd, 390.21929. found, 390.21990.

### Step 3

### 6-((1-(2-(3-Fluoro-6-(4-hydroxybutoxy)-1,5-naphthyridin-4-yl)ethyl)-2-oxabicyclo[2.2.2]octan-4-ylamino)methyl)-2H-pyrido[3,2-b][1,4]oxazin-3(4H)-one

The title compound 6-((1-(2-(3-fluoro-6-(4-hydroxybutoxy)-1,5-naphthyridin-4-yl)ethyl)-2-oxabicyclo[2.2.2]octan-4-ylamino)methyl)-2H-pyrido[3,2-b][1,4]oxazin-3(4H)-one (61.1 mg) was prepared from 4-(8-(2-(4-amino-2-oxabicyclo[2.2.2]octan-1-yl)ethyl)-7-fluoro-1,5-naphthyridin-2-yloxy)butan-1-ol (60.0 mg) and I (28.8 mg) in the same manner as described for Step 3 of EXAMPLE 1.

¹H NMR (DMSO-d₆): δ 1.50-1.94 (m, 15H), 3.03-3.14 (m, 2H), 3.46 (q, J=6.1 Hz, 2H), 3.58 (s, 2H), 3.62 (s, 2H), 4.42-4.53 (m, 3H), 4.59 (s, 2H), 7.01 (d, J=7.9 Hz, 1H), 7.19 (d, J=9.2 Hz, 1H), 7.28 (d, J=7.9 Hz, 1H), 8.24 (d, J=8.6 Hz, 1H), 8.73 (s, 1H), 11.15 (s, 1H).

MS (ESI⁺) m/z: 552 (MH⁺).

HRMS (ESI⁺) for C₂₉H₃₅FN₅O₅ (MH⁺): calcd, 552.26222. found, 552.26317.

### Example 37

### 6-((1-(2-(3-Fluoro-6-(5-hydroxypentyloxy)-1,5-naphthyridin-4-yl)ethyl)-2-oxabicyclo[2.2.2]octan-4-ylamino)methyl)-2H-pyrido[3,2-b][1,4]oxazin-3(4H)-one

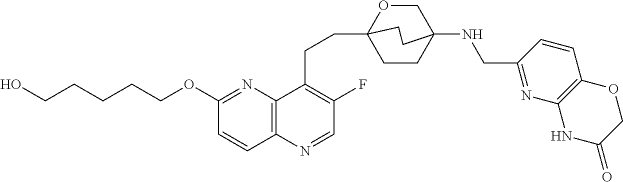 [SMILES: O=C1COc2ccc(CNC34CCC(CCc5c(F)cnc6ccc(OCCCCCO)nc56)(CC3)OC4)nc2N1]

### Step 1

### tert-Butyl 1-(2-(3-Fluoro-6-(5-(tetrahydro-2H-pyran-2-yloxy)pentyloxy)-1,5-naphthyridin-4-yl)ethyl)-2-oxabicyclo[2.2.2]octan-4-ylcarbamate

The title compound tert-butyl 1-(2-(3-fluoro-6-(5-(tetrahydro-2H-pyran-2-yloxy)pentyloxy)-1,5-naphthyridin-4-yl)ethyl)-2-oxabicyclo[2.2.2]octan-4-ylcarbamate (132 mg) was prepared from V (100 mg) and 2-(6-bromohexyloxy)tetrahydro-2H-pyran (66.2 mg) in the same manner as described for Step 1 of EXAMPLE 32.

¹H NMR (CDCl₃): δ 1.43 (s, 9H), 1.61-2.16 (m, 22H), 3.14-3.21 (m, 2H), 3.40-3.53 (m, 2H), 3.76-3.83 (m, 1H) 3.84-3.91 (m, 1H), 3.96 (s, 2H), 4.30 (s, 1H), 4.48 (t, J=6.4 Hz, 2H), 4.57-4.61 (m, 1H), 7.03 (d, J=9.2 Hz, 1H), 8.14 (d, J=9.2 Hz, 1H), 8.58 (s, 1H).

MS (ESI⁺) m/z: 588 (MH⁺).

HRMS (ESI⁺) for C₃₂H₄₇FN₃O₆ (MH⁺): calcd, 588.34489. found, 588.34493.

### Step 2

### 5-(8-(2-(4-Amino-2-oxabicyclo[2.2.2]octan-1-yl)ethyl)-7-fluoro-1,5-naphthyridin-2-yloxy)pentan-1-ol

The title compound 5-(8-(2-(4-amino-2-oxabicyclo[2.2.2]octan-1-yl)ethyl)-7-fluoro-1,5-naphthyridin-2-yloxy)pentan-1-ol (84.1 mg) was prepared from tert-butyl 1-(2-(3-fluoro-6-(5-(tetrahydro-2H-pyran-2-yloxy)pentyloxy)-1,5-naphthyridin-4-yl)ethyl)-2-oxabicyclo[2.2.2]octan-4-ylcarbamate (125 mg) in the same manner as described for Step 2 of EXAMPLE 32.

¹H NMR (DMSO-d₆): δ 1.53-2.09 (m, 16H), 3.05-3.10 (m, 2H), 3.41 (t, J=6.1 Hz, 2H), 3.48 (s, 2H), 4.39 (s, 1H), 4.56 (t, J=6.7 Hz, 2H), 7.19 (d, J=9.2 Hz, 1H), 8.24 (d, J=8.6 Hz, 1H), 8.72 (s, 1H).

MS (ESI⁺) m/z: 404 (MH⁺).

HRMS (ESI⁺) for C₂₂H₃₁FN₃O₃ (MH⁺): calcd, 404.23494. found, 404.23568.

### Step 3

### 6-((1-(2-(3-Fluoro-6-(5-hydroxypentyloxy)-1,5-naphthyridin-4-yl)ethyl)-2-oxabicyclo[2.2.2]octan-4-ylamino)methyl)-2H-pyrido[3,2-b][1,4]oxazin-3(4H)-one

The title compound 6-((1-(2-(3-fluoro-6-(5-hydroxypentyloxy)-1,5-naphthyridin-4-yl)ethyl)-2-oxabicyclo[2.2.2]octan-4-ylamino)methyl)-2H-pyrido[3,2-b][1,4]oxazin-3(4H)-one (60.0 mg) was prepared from 5-(8-(2-(4-amino-2-oxabicyclo[2.2.2]octan-1-yl)ethyl)-7-fluoro-1,5-naphthyridin-2-yloxy)pentan-1-ol (80.0 mg) and I (37.1 mg) in the same manner as described for Step 3 of EXAMPLE 1.

¹H NMR (DMSO-d₆): δ 1.38-1.94 (m, 16H), 3.04-3.24 (m, 2H), 3.41 (q, J=5.2 Hz, 2H), 3.58 (s, 2H), 3.62 (d, J=4.3 Hz, 2H), 4.39 (t, J=5.2 Hz, 1H), 4.46 (d, J=7.0 Hz, 2H), 4.59 (s, 2H), 7.01 (d, J=7.9 Hz, 1H), 7.19 (d, J=9.2 Hz, 1H), 7.28 (d, J=7.9 Hz, 1H), 8.24 (d, J=9.2 Hz, 1H), 8.73 (s, 1H), 11.16 (s, 1H).

MS (ESI⁺) m/z: 566 (MH⁺).

HRMS (ESI⁺) for C₃₀H₃₇FN₅O₅ (MH⁺): calcd, 566.27787. found, 566.27696.

### Example 38

### 6-((1-(2-(6-(3-Aminopropoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

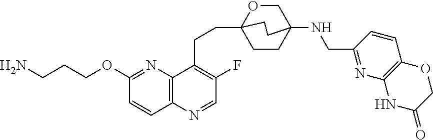 [SMILES: NCCCOc1ccc2ncc(F)c(CCC34CCC(NCc5ccc6c(n5)NC(=O)CO6)(CC3)CO4)c2n1]

### Step 1

### tert-Butyl 1-(2-(6-(2-(Benzyloxycarbonylamino)propoxy)-3-fluoro-1,5-naphthyridin-4-yl)ethyl)-2-oxabicyclo[2.2.2]octan-4-ylcarbamate

The title compound 6-{[(1-{2-[6-(3-aminopropoxy)-3-fluoro-1,5-naphthyridin-4-yl]ethyl}-2-oxabicyclo[2.2.2]oct-4-yl)amino]methyl}-2H-pyrido[3,2-b][1,4]oxazin-3(4H)-one (132 mg) was prepared from V (100 mg) and benzyl 3-bromopropylcarbamate (86.1 mg) in the same manner as described for Step 1 of EXAMPLE 32.

¹H NMR (CDCl₃): δ 1.43 (s, 9H), 1.67-2.15 (m, 12H), 3.15 (t, J=8.3 Hz, 2H), 3.42 (d, J=6.3 Hz, 2H), 3.94 (s, 2H), 4.25 (s, 1H), 4.56 (q, J=7.5 Hz, 2H), 5.11 (s, 2H), 5.24 (s, 1H), 7.02 (d, J=9.2 Hz, 1H), 7.28-7.40 (m, 5H), 8.15 (d, J=9.2 Hz, 1H), 8.59 (s, 1H).

MS (ESI⁺) m/z: 609 (MH⁺).

HRMS (ESI⁺) for C₃₃H₄₂FN₄O₆ (MH⁺): calcd, 609.30884. found, 609.30809.

### Step 2

### Benzyl 3-(8-(2-(4-Amino-2-oxabicyclo[2.2.2]octan-1-yl)ethyl)-7-fluoro-1,5-naphthyridin-2-yloxy)propylcarbamate

The title compound benzyl 3-(8-(2-(4-amino-2-oxabicyclo[2.2.2]octan-1-yl)ethyl)-7-fluoro-1,5-naphthyridin-2-yloxy)propylcarbamate (102 mg) was prepared from tert-butyl 1-(2-(6-(2-(benzyloxycarbonylamino)propoxy)-3-fluoro-1,5-naphthyridin-4-yl)ethyl)-2-oxabicyclo[2.2.2]octan-4-ylcarbamate (125 mg) in the same manner as described for Step 2 of EXAMPLE 32.

¹H NMR (DMSO-d₆): δ 1.46-2.01 (m, 12H), 3.02-3.13 (m, 2H), 3.14-3.24 (m, 2H), 3.45 (s, 2H), 4.48 (t, J=6.4 Hz, 2H), 4.99 (s, 2H), 7.19 (d, J=9.2 Hz, 1H), 7.22-7.42 (m, 5H), 8.24 (d, J=9.2 Hz, 1H), 8.73 (s, 1H).

MS (ESI⁺) m/z: 509 (MH⁺).

HRMS (ESI⁺) for C₂₈H₃₄FN₄O₄ (MH⁺): calcd, 509.25641. found, 509.25682.

### Step 3

### Benzyl 3-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)propylcarbamate

The title compound benzyl 3-(7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)propylcarbamate (106 mg) was prepared from benzyl 3-(8-(2-(4-amino-2-oxabicyclo[2.2.2]octan-1-yl)ethyl)-7-fluoro-1,5-naphthyridin-2-yloxy)propylcarbamate (95.0 mg) and I (34.9 mg) in the same manner as described for Step 3 of EXAMPLE 1.

¹H NMR (DMSO-d₆): δ 1.56-2.01 (m, 13H), 3.04-3.13 (m, 2H), 3.15-3.23 (m, 2H), 3.57 (s, 2H), 3.62 (s, 2H), 4.48 (t, J=6.4 Hz, 2H), 4.58 (s, 2H), 4.99 (s, 2H), 7.00 (d, J=7.9 Hz, 1H), 7.19 (d, J=9.2 Hz, 1H), 7.24-7.38 (m, 6H), 8.24 (d, J=9.2 Hz, 1H), 8.73 (s, 1H), 11.15 (s, 1H).

MS (ESI⁺) m/z: 671 (MH⁺).

HRMS (ESI⁺) for C₃₆H₄₀FN₆O₆ (MH⁺): calcd, 671.29933. found, 671.29952.

### Step 4

### 6-((1-(2-(6-(3-Aminopropoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

A suspension of benzyl 3-(7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)propylcarbamate (95.0 mg) and 10% PdC (19.0 mg) in methanol (1.4 mL) and acetic acid (0.3 mL) was stirred at room temperature for 3 hours under H₂ atmosphere (1 kg/cm²). After the insoluble materials were filtered off, the filtrate was concentrated in vacuo to give 6-((1-(2-(6-(3-aminopropoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one (65.7 mg).

¹H NMR (DMSO-d₆): δ 1.54-1.94 (m, 13H), 2.70 (t, J=7.0 Hz, 2H), 3.04-3.14 (m, 2H), 3.58 (s, 2H), 3.62 (s, 2H), 4.53 (d, J=6.7 Hz, 2H), 4.59 (s, 2H), 7.01 (d, J=7.9 Hz, 1H), 7.19 (d, J=9.2 Hz, 1H), 7.28 (d, J=7.9 Hz, 1H), 8.24 (d, J=9.2 Hz, 1H), 8.73 (s, 1H).

MS (ESI⁺) m/z: 537 (MH⁺).

HRMS (ESI⁺) for C₂₈H₃₄FN₆O₄ (MH⁺): calcd, 537.26256. found, 537.26260.

### Example 39

### 6-((1-(2-(6-(4-Aminobutoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

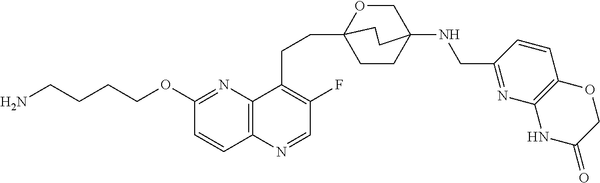 [SMILES: NCCCCOc1ccc2ncc(F)c(CCC34CCC(NCc5ccc6c(n5)NC(=O)CO6)(CC3)CO4)c2n1]

### Step 1

### tert-Butyl 1-(2-(6-(2-(Benzyloxycarbonylamino)butoxy)-3-fluoro-1,5-naphthyridin-4-yl)ethyl)-2-oxabicyclo[2.2.2]octan-4-ylcarbamate

The title compound 6-{[(1-{2-[6-(4-aminobutoxy)-3-fluoro-1,5-naphthyridin-4-yl]ethyl}-2-oxabicyclo[2.2.2]oct-4-yl)amino]methyl}-2H-pyrido[3,2-b][1,4]oxazin-3(4H)-one (125 mg) was prepared from V (100 mg) and benzyl 4-bromobutylcarbamate (75.4 mg) in the same manner as described for Step 1 of EXAMPLE 32.

¹H NMR (DMSO-d₆): δ 1.42 (s, 9H), 1.67-2.12 (m, 14H), 3.13-3.17 (m, 2H), 3.28-3.35 (m, 2H), 3.96 (s, 2H), 4.30 (s, 1H), 4.50 (t, J=6.8 Hz, 2H), 5.09 (s, 3H), 7.02 (d, J=9.2 Hz, 1H), 7.29-7.34 (m, 5H), 8.15 (d, J=9.2 Hz, 1H), 8.58 (s, 1H).

MS (ESI⁺) m/z: 623 (MH⁺).

HRMS (ESI⁺) for C₃₄H₄₄FN₄O₆ (MH⁺): calcd, 623.32449. found, 623.32404.

### Step 2

Benzyl 4-(8-(2-(4-Amino-2-oxabicyclo[2.2.2]octan-1-yl)ethyl)-7-fluoro-1,5-naphthyridin-2-yloxy)butylcarbamate

The title compound benzyl 4-(8-(2-(4-amino-2-oxabicyclo[2.2.2]octan-1-yl)ethyl)-7-fluoro-1,5-naphthyridin-2-yloxy)butylcarbamate (101 mg) was prepared from tert-butyl 1-(2-(6-(2-(benzyloxycarbonylamino)propoxy)-3-fluoro-1,5-naphthyridin-4-yl)ethyl)-2-oxabicyclo[2.2.2]octan-4-ylcarbamate (120 mg) in the same manner as described for Step 2 of EXAMPLE 32.

¹H NMR (DMSO-d₆): δ 1.36-1.92 (m, 14H), 3.02-3.12 (m, 4H), 3.27-3.39 (m, 2H), 3.45 (s, 2H), 4.46 (t, J=6.7 Hz, 2H), 5.00 (s, 2H), 7.19 (d, J=9.2 Hz, 1H), 7.29-7.34 (m, 5H), 8.24 (d, J=9.2 Hz, 1H), 8.73 (s, 1H).

MS (ESI⁺) m/z: 523 (MH⁺).

HRMS (ESI⁺) for C₂₉H₃₆FN₄O₄ (MH⁺): calcd, 523.27206. found, 523.27287.

### Step 3

### Benzyl 4-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butylcarbamate

The title compound benzyl 4-(7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butylcarbamate (94.6 mg) was prepared from benzyl 4-(8-(2-(4-amino-2-oxabicyclo[2.2.2]octan-1-yl)ethyl)-7-fluoro-1,5-naphthyridin-2-yloxy)butylcarbamate (95.0 mg) and I (34.0 mg) in the same manner as described for Step 3 of EXAMPLE 1.

¹H NMR (DMSO-d₆): δ 1.49-1.93 (m, 15H), 3.01-3.14 (m, 4H), 3.58 (s, 2H), 3.62 (s, 2H), 4.47 (t, J=6.8 Hz, 2H), 4.58 (s, 2H), 4.99 (s, 2H), 7.00 (d, J=7.9 Hz, 1H), 7.19 (d, J=9.2 Hz, 1H), 7.22-7.35 (m, 6H), 8.25 (d, J=9.2 Hz, 1H), 8.73 (s, 1H), 11.15 (s, 1H).

MS (ESI⁺) m/z: 685 (MH⁺).

HRMS (ESI⁺) for C₃₇H₄₂FN₆O₆ (MH⁺): calcd, 685.31498. found, 685.31448.

### Step 4

### 6-((1-(2-(6-(4-Aminobutoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

The title compound 6-((1-(2-(6-(4-aminobutoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one (51.5 mg) was prepared from benzyl 4-(7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butylcarbamate (90.0 mg) in the same manner as described for Step 4 of EXAMPLE 38.

¹H NMR (DMSO-d₆): δ 1.43-1.94 (m, 16H), 2.61 (t, J=7.0 Hz, 2H), 3.04-3.14 (m, 2H), 3.58 (s, 2H), 3.62 (s, 2H), 4.46 (t, J=6.7 Hz, 2H), 4.59 (s, 2H), 4.96 (br, 1H), 7.01 (d, J=7.9 Hz, 1H), 7.19 (d, J=9.2 Hz, 1H), 7.28 (d, J=7.9 Hz, 1H), 8.24 (d, J=9.2 Hz, 1H), 8.73 (s, 1H).

MS (ESI⁺) m/z: 551 (MH⁺).

HRMS (ESI⁺) for C₂₉H₃₆FN₆O₄ (MH⁺): calcd, 551.27821. found, 551.27796.

### Example 40

### 6-((1-(2-(6-(2-Aminoethoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

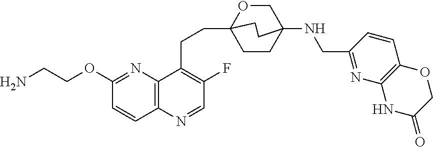 [SMILES: NCCOc1ccc2ncc(F)c(CCC34CCC(NCc5ccc6c(n5)NC(=O)CO6)(CC3)CO4)c2n1]

### Step 1

### tert-Butyl 1-(2-(6-(2-Aminoethoxy)-3-fluoro-1,5-naphthyridin-4-yl)ethyl)-2-oxabicyclo[2.2.2]octan-4-ylcarbamate

The title compound tert-butyl 1-(2-(6-(2-aminoethoxy)-3-fluoro-1,5-naphthyridin-4-yl)ethyl)-2-oxabicyclo[2.2.2]octan-4-ylcarbamate (33.4 mg) was prepared from V (100 mg) and (9H-fluoren-9-yl)methyl 2-bromoethylcarbamate (96.0 mg) in the same manner as described for Step 1 of EXAMPLE 32.

¹H NMR (CDCl₃): δ 1.43 (s, 9H), 1.68-2.20 (m, 10H), 3.14-3.19 (m, 2H), 3.97 (s, 2H), 4.29 (s, 1H), 4.53 (t, J=5.2 Hz, 2H), 7.08 (d, J=8.6 Hz, 1H), 8.17 (d, J=8.6 Hz, 1H), 8.59 (s, 1H).

MS (ESI⁺) m/z: 461 (MH⁺).

HRMS (ESI⁺) for C₂₄H₃₄FN₄O₄ (MH⁺): calcd, 461.25641. found, 461.25704.

### Step 2

### tert-Butyl 1-(2-(6-(2-(Benzyloxycarbonylamino)ethoxy)-3-fluoro-1,5-naphthyridin-4-yl)ethyl)-2-oxabicyclo[2.2.2]octan-4-ylcarbamate

To a solution of tert-butyl 1-(2-(6-(2-aminoethoxy)-3-fluoro-1,5-naphthyridin-4-yl)ethyl)-2-oxabicyclo[2.2.2]octan-4-ylcarbamate (49.0 mg) in tetrahydrofuran (0.5 mL) and saturated sodium hydrogen carbonate solution (0.5 mL) was added benzyl chloroformate (21.8 mg) under cooling with ice, the mixture was stirred at room temperature for 1 hour. After dilution of the mixture with water, the mixture was extracted with dichloromethane. The organic extracts were washed with brine, dried over anhydrous sodium sulfate, filtered, and then concentrated in vacuo. Flash chromatography (silica, hexane:ethyl acetate=3:2) of the residue gave tert-butyl 1-(2-(6-(2-(benzyloxycarbonylamino)ethoxy)-3-fluoro-1,5-naphthyridin-4-yl)ethyl)-2-oxabicyclo[2.2.2]octan-4-ylcarbamate (51.1 mg).

¹H NMR (CDCl₃): δ 1.44 (s, 9H), 1.55-2.13 (m, 10H), 3.08-3.20 (m, 2H), 3.16 (q, J=5.5 Hz, 2H), 3.95 (s, 2H), 4.19 (s, 1H), 4.61 (t, J=6.1 Hz, 2H), 5.08 (s, 2H), 5.67 (s, 1H), 7.04 (d, J=8.6 Hz, 1H), 7.30 (s, 5H), 8.17 (d, J=9.2 Hz, 1H), 8.60 (s, 1H).

MS (ESI⁺) m/z: 595 (MH⁺).

HRMS (ESI⁺) for C₃₂H₄₀FN₄O₆ (MH⁺): calcd, 595.29319. found, 595.26367.

### Step 3

### Benzyl 2-(8-(2-(4-Amino-2-oxabicyclo[2.2.2]octan-1-yl)ethyl)-7-fluoro-1,5-naphthyridin-2-yloxy)ethylcarbamate

The title compound benzyl 2-(8-(2-(4-amino-2-oxabicyclo[2.2.2]octan-1-yl)ethyl)-7-fluoro-1,5-naphthyridin-2-yloxy)ethylcarbamate (37.1 mg) was prepared from tert-butyl 1-(2-(6-(2-(benzyloxycarbonylamino)ethoxy)-3-fluoro-1,5-naphthyridin-4-yl)ethyl)-2-oxabicyclo[2.2.2]octan-4-ylcarbamate (49.0 mg) in the same manner as described for Step 2 of EXAMPLE 32.

¹H NMR (CDCl₃): δ 1.43-1.82 (m, 8H), 1.82-2.04 (m, 2H), 3.09-3.21 (m, 2H), 3.49 (s, 2H), 3.64-3.71 (m, 4H), 4.54-4.65 (m, 2H), 5.08 (s, 2H), 5.73 (s, 1H), 7.04 (d, J=9.2 Hz, 1H), 7.32 (s, 5H), 8.18 (d, J=9.2 Hz, 1H), 8.60 (s, 1H).

MS (ESI⁺) m/z: 495 (MH⁺).

HRMS (ESI⁺) for C₂₇H₃₂FN₄O₄ (MH⁺): calcd, 495.24076. found, 495.24115.

### Step 4

### Benzyl 2-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)ethylcarbamate

The title compound benzyl 2-(7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)ethylcarbamate (39.9 mg) was prepared from benzyl 2-(8-(2-(4-amino-2-oxabicyclo[2.2.2]octan-1-yl)ethyl)-7-fluoro-1,5-naphthyridin-2-yloxy)ethylcarbamate (36.0 mg) and I (13.6 mg) in the same manner as described for Step 3 of EXAMPLE 1.

¹H NMR (DMSO-d₆): δ 1.52-1.92 (m, 10H), 3.06-3.11 (m, 2H), 3.49 (t, J=11.0 Hz, 2H), 3.58 (s, 2H), 3.62 (s, 2H), 4.50 (t, J=5.5 Hz, 2H), 4.58 (s, 2H), 5.00 (s, 2H), 7.00 (d, J=7.9 Hz, 1H), 7.18 (d, J=9.2 Hz, 1H), 7.24-7.36 (m, 6H), 7.49 (t, J=5.5 Hz, 1H), 8.26 (d, J=9.2 Hz, 1H), 8.74 (s, 1H), 11.14 (s, 1H).

MS (ESI⁺) m/z: 657 (MH⁺).

HRMS (ESI⁺) for C₃₅H₃₈FN₆O₆ (MH⁺): calcd, 657.28368. found, 657.28319.

### Step 5

### 6-((1-(2-(6-(2-Aminoethoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

The title compound 6-((1-(2-(6-(2-aminoethoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one (17.8 mg) was prepared from benzyl 2-(7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)ethylcarbamate (35.0 mg) in the same manner as described for Step 4 of EXAMPLE 38.

¹H NMR (DMSO-d₆): δ 1.55-1.93 (m, 10H), 3.02-3.13 (m, 4H), 3.58 (s, 2H), 3.62 (s, 2H), 4.41 (t, J=5.8 Hz, 2H), 4.59 (s, 2H), 7.01 (d, J=7.9 Hz, 1H), 7.21 (d, J=9.2 Hz, 1H), 7.28 (d, J=7.9 Hz, 1H), 8.25 (d, J=9.2 Hz, 1H), 8.73 (s, 1H).

MS (ESI⁺) m/z: 523 (MH⁺).

HRMS (ESI⁺) for C₂₇H₃₂FN₆O₄ (MH⁺): calcd, 523.24691. found, 523.24628.

### Example 41

### 6-((1-(2-(6-(5-Aminopentyloxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

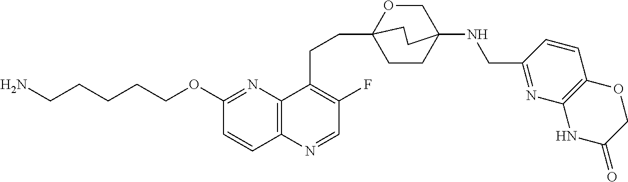 [SMILES: NCCCCCOc1ccc2ncc(F)c(CCC34CCC(NCc5ccc6c(n5)NC(=O)CO6)(CC3)CO4)c2n1]

### Step 1

### tert-Butyl 1-(2-(6-(2-(Benzyloxycarbonylamino)pentyloxy)-3-fluoro-1,5-naphthyridin-4-yl)ethyl)-2-oxabicyclo[2.2.2]octan-4-ylcarbamate

The title compound tert-butyl 1-(2-(6-(2-(benzyloxycarbonylamino)pentyloxy)-3-fluoro-1,5-naphthyridin-4-yl)ethyl)-2-oxabicyclo[2.2.2]octan-4-ylcarbamate (139 mg) was prepared from V (100 mg) and benzyl 5-bromopentylcarbamate (93.5 mg) in the same manner as described for Step 1 of EXAMPLE 32.

¹H NMR (CDCl₃): δ 1.42 (s, 9H), 1.48-2.15 (m, 16H), 3.11-3.19 (m, 2H), 3.24 (q, J=6.8 Hz, 2H), 3.95 (s, 2H), 4.33 (s, 1H), 4.48 (t, J=6.7 Hz, 2H), 5.01 (s, 1H), 5.10 (s, 2H), 7.02 (d, J=8.6 Hz, 1H), 7.30-7.40 (m, 5H), 8.14 (d, J=9.2 Hz, 1H), 8.58 (s, 1H).

MS (ESI⁺) m/z: 637 (MH⁺).

HRMS (ESI⁺) for C₃₅H₄₆FN₄O₆ (MH⁺): calcd, 637.34014. found, 637.34099.

### Step 2

### Benzyl 5-(8-(2-(4-Amino-2-oxabicyclo[2.2.2]octan-1-yl)ethyl)-7-fluoro-1,5-naphthyridin-2-yloxy)pentylcarbamate

The title compound benzyl 5-(8-(2-(4-amino-2-oxabicyclo[2.2.2]octan-1-yl)ethyl)-7-fluoro-1,5-naphthyridin-2-yloxy)pentylcarbamate (108 mg) was prepared from tert-butyl 1-(2-(6-(2-(benzyloxycarbonylamino)pentyloxy)-3-fluoro-1,5-naphthyridin-4-yl)ethyl)-2-oxabicyclo[2.2.2]octan-4-ylcarbamate (135 mg) in the same manner as described for Step 2 of EXAMPLE 32.

¹H NMR (DMSO-d₆): δ 1.74-1.92 (m, 18H), 3.16-3.13 (m, 4H), 3.45 (s, 2H), 4.45 (t, J=6.7 Hz, 2H), 4.99 (s, 2H), 7.18 (d, J=9.2 Hz, 1H), 7.19-7.34 (m, 6H), 8.24 (d, J=9.2 Hz, 1H), 8.72 (s, 1H).

MS (ESI⁺) m/z: 573 (MH⁺).

HRMS (ESI⁺) for C₃₀H₃₈FN₄O₄ (MH⁺): calcd, 573.28771. found, 573.28764.

### Step 3

### Benzyl 5-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)pentylcarbamate

The title compound benzyl 5-(7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)pentylcarbamate (111 mg) was prepared from benzyl 5-(8-(2-(4-amino-2-oxabicyclo[2.2.2]octan-1-yl)ethyl)-7-fluoro-1,5-naphthyridin-2-yloxy)pentylcarbamate (100 mg) and I (34.9 mg) in the same manner as described for Step 3 of EXAMPLE 1.

¹H NMR (DMSO-d₆): δ 1.35-1.94 (m, 16H), 3.01 (q, J=6.5 Hz, 2H), 3.06-3.11 (m, 2H), 3.58 (m, 2H), 3.62 (m, 2H), 4.45 (t, J=6.7 Hz, 2H), 4.58 (s, 2H), 4.98 (s, 2H), 7.00 (d, J=7.9 Hz, 1H), 7.19 (d, J=9.2 Hz, 1H), 7.21-7.38 (m, 7H), 8.24 (d, J=8.6 Hz, 1H), 8.73 (s, 1H), 11.14 (s, 1H).

MS (ESI⁺) m/z: 699 (MH⁺).

HRMS (ESI⁺) for C₃₈H₄₄FN₆O₆ (MH⁺): calcd, 699.33063. found, 699.32998.

### Step 4

### 6-((1-(2-(6-(5-Aminopentyloxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

The title compound 6-((1-(2-(6-(5-aminopentyloxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one (73.3 mg) was prepared from benzyl 5-(7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)pentylcarbamate (105 mg) in the same manner as described for Step 4 of EXAMPLE 38.

¹H NMR (DMSO-d₆): δ 1.32-1.95 (m, 16H), 2.54 (t, J=6.1 Hz, 2H), 3.06-3.01 (m, 2H), 3.33 (brs, 2H), 3.58 (s, 2H), 3.62 (s, 2H), 4.46 (t, J=6.8 Hz, 2H), 4.59 (s, 2H), 7.00 (d, J=8.0 Hz, 1H), 7.19 (d, J=9.2 Hz, 1H), 7.27 (d, J=8.6 Hz, 1H), 8.24 (d, J=9.2 Hz, 1H), 8.73 (s, 1H).

MS (ESI⁺) m/z: 565 (MH⁺).

HRMS (ESI⁺) for C₃₀H₃₈FN₆O₄ (MH⁺): calcd, 565.29386. found, 565.29324.

### Example 42

### 6-((1-(2-(3-Fluoro-6-((1-(hydroxymethyl)cyclopropyl)methoxy)-1,5-naphthyridin-4-yl)ethyl)-2-oxabicyclo[2.2.2]octan-4-ylamino)methyl)-2H-pyrido[3,2-b][1,4]oxazin-3(4H)-one

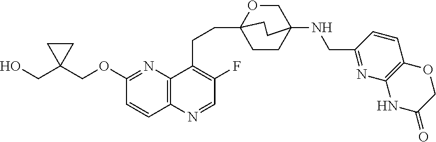 [SMILES: O=C1COc2ccc(CNC34CCC(CCc5c(F)cnc6ccc(OCC7(CO)CC7)nc56)(CC3)OC4)nc2N1]

### Step 1

### tert-Butyl 1-(2-(3-Fluoro-6-((1-((tetrahydro-2H-pyran-2-yloxy)methyl)cyclopropyl)methoxy)-1,5-naphthyridin-4-yl)ethyl)-2-oxabicyclo[2.2.2]octan-4-ylcarbamate

The title compound tert-butyl 1-(2-(3-fluoro-6-((1-((tetrahydro-2H-pyran-2-yloxy)methyl)cyclopropyl)methoxy)-1,5-naphthyridin-4-yl)ethyl)-2-oxabicyclo[2.2.2]octan-4-ylcarbamate (128 mg) was prepared from V (100 mg) and W (65.6 mg) in the same manner as described for Step 1 of EXAMPLE 32.

¹H NMR (CDCl₃): δ 0.66-0.71 (s, 4H), 1.45 (s, 9H), 1.46-2.11 (m, 16H), 3.10-3.19 (m, 2H), 3.43-4.48 (m, 1H), 3.50 (d, J=10.4 Hz, 1H), 3.76 (d, J=10.4 Hz, 1H), 3.79-3.87 (m, 1H), 3.95 (s, 2H), 4.29 (brs, 1H), 4.43 (d, J=11.0 Hz, 1H), 4.48 (d, J=11.6 Hz, 1H), 4.65 (t, J=3.7 Hz, 1H), 7.07 (d, J=9.2 Hz, 1H), 8.15 (d, J=9.2 Hz, 1H), 8.57 (s, 1H).

MS (ESI⁺) m/z: 586 (MH⁺).

HRMS (ESI⁺) for C₃₂H₄₅FN₃O₆ (MH⁺): calcd, 586.32924. found, 586.32859.

### Step 2

### (1-((8-(2-(4-Amino-2-oxabicyclo[2.2.2]octan-1-yl)ethyl)-7-fluoro-1,5-naphthyridin-2-yloxy)methyl)cyclopropyl)methanol

The title compound (1-((8-(2-(4-amino-2-oxabicyclo[2.2.2]octan-1-yl)ethyl)-7-fluoro-1,5-naphthyridin-2-yloxy)methyl)cyclopropyl)methanol (40.1 mg) was prepared from tert-butyl 1-(2-(3-fluoro-6-((1-((tetrahydro-2H-pyran-2-yloxy)methyl)cyclopropyl)methoxy)-1,5-naphthyridin-4-yl)ethyl)-2-oxabicyclo[2.2.2]octan-4-ylcarbamate (120 mg) in the same manner as described for Step 2 of EXAMPLE 32.

¹H NMR (DMSO-d₆): δ 0.55 (s, 4H), 1.47-2.12 (m, 12H), 3.00-3.14 (m, 2H), 3.44 (d, J=5.5 Hz, 2H), 3.48 (s, 2H), 4.41 (s, 2H), 4.66 (t, J=5.5 Hz, 1H), 7.25 (d, J=8.6 Hz, 1H), 8.25 (d, J=9.2 Hz, 1H), 8.73 (s, 1H).

MS (ESI⁺) m/z: 402 (MH⁺).

HRMS (ESI⁺) for C₂₂H₂₉FN₃O₃ (MH⁺): calcd, 402.21929. found, 402.21983.

### Step 3

### 6-((1-(2-(3-Fluoro-6-((1-(hydroxymethyl)cyclopropyl)methoxy)-1,5-naphthyridin-4-yl)ethyl)-2-oxabicyclo[2.2.2]octan-4-ylamino)methyl)-2H-pyrido[3,2-b][1,4]oxazin-3(4H)-one

The title compound 6-((1-(2-(3-fluoro-6-((1-(hydroxymethyl)cyclopropyl)methoxy)-1,5-naphthyridin-4-yl)ethyl)-2-oxabicyclo[2.2.2]octan-4-ylamino)methyl)-2H-pyrido[3,2-b][1,4]oxazin-3(4H)-one (28.3 mg) was prepared from (1-((8-(2-(4-amino-2-oxabicyclo[2.2.2]octan-1-yl)ethyl)-7-fluoro-1,5-naphthyridin-2-yloxy)methyl)cyclopropyl)methanol (34.2 mg) and I (15.9 mg) in the same manner as described for Step 3 of EXAMPLE 1.

¹H NMR (DMSO-d₆): δ 0.54 (s, 4H), 1.54-1.72 (m, 8H), 1.77-1.95 (m, 3H), 3.01-3.14 (m, 2H), 3.39 (d, J=5.4 Hz, 2H), 3.59 (s, 2H), 3.64 (s, 2H), 4.40 (s, 2H), 4.59 (s, 2H), 4.65 (t, J=5.5 Hz, 1H), 7.01 (d, J=7.9 Hz, 1H), 7.22 (d, J=9.1 Hz, 1H), 7.29 (d, J=7.9 Hz, 1H), 8.24 (d, J=9.1 Hz, 1H), 8.72 (s, 1H), 11.16 (brs, 1H).

MS (ESI⁺) m/z: 564 (MH⁺).

HRMS (ESI⁺) for C₃₀H₃₅FN₅O₅ (MH⁺): calcd, 564.26222. found, 564.26133.

### Example 43

### 6-((1-(2-(6-((1-(Aminomethyl)cyclopropyl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

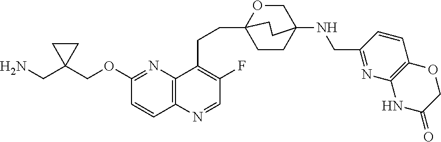 [SMILES: NCC1(COc2ccc3ncc(F)c(CCC45CCC(NCc6ccc7c(n6)NC(=O)CO7)(CC4)CO5)c3n2)CC1]

### Step 1

### tert-Butyl 1-(2-(6-((1-(Benzyloxycarbonylaminomethyl)cyclopropyl)methoxy)-3-fluoro-1,5-naphthyridin-4-yl)ethyl)-2-oxabicyclo[2.2.2]octan-4-ylcarbamate

The title compound tert-butyl 1-(2-(6-((1-(benzyloxycarbonylaminomethyl)cyclopropyl)methoxy)-3-fluoro-1,5-naphthyridin-4-yl)ethyl)-2-oxabicyclo[2.2.2]octan-4-ylcarbamate (112 mg) was prepared from V (82.3 mg) and X (65.0 mg) in the same manner as described for Step 1 of EXAMPLE 32.

¹H NMR (CDCl₃): δ 0.68 (s, 4H), 1.44 (s, 9H), 1.65-1.76 (m, 4H), 1.79-2.12 (m, 6H), 3.08-3.16 (m, 2H), 3.33 (d, J=5.4 Hz, 2H) 3.94 (s, 2H), 4.26 (brs, 1H), 4.43 (s, 2H), 5.07 (s, 2H), 5.36 (brs, 1H), 7.04 (d, J=8.5 Hz, 1H), 7.29-7.37 (m, 5H), 8.15 (d, J=9.1 Hz, 1H), 8.59 (s, 1H).

MS (ESI⁺) m/z: 635 (MH⁺).

HRMS (ESI⁺) for C₃₅H₄₄FN₄O₆ (MH⁺): calcd, 635.32449. found, 635.32546.

### Step 2

### Benzyl (1-((8-(2-(4-Amino-2-oxabicyclo[2.2.2]octan-1-yl)ethyl)-7-fluoro-1,5-naphthyridin-2-yloxy)methyl)cyclopropyl)methylcarbamate

The title compound benzyl (1-((8-(2-(4-amino-2-oxabicyclo[2.2.2]octan-1-yl)ethyl)-7-fluoro-1,5-naphthyridin-2-yloxy)methyl)cyclopropyl)methylcarbamate (88.5 mg) was prepared from tert-butyl 1-(2-(6-((1-(benzyloxycarbonylaminomethyl)cyclopropyl)methoxy)-3-fluoro-1,5-naphthyridin-4-yl)ethyl)-2-oxabicyclo[2.2.2]octan-4-ylcarbamate (105 mg) in the same manner as described for Step 2 of EXAMPLE 1.

¹H NMR (CDCl₃): δ 0.68 (s, 4H), 1.57-1.76 (m, 8H), 1.91-2.05 (m, 2H), 3.09-3.17 (m, 2H), 3.33 (d, J=6.1 Hz, 2H), 3.62 (s, 2H), 4.44 (s, 2H), 5.08 (s, 2H), 5.37 (br, 1H), 7.05 (d, J=8.5 Hz, 1H), 7.30-7.36 (m, 5H), 8.16 (d, J=8.5 Hz, 1H), 8.59 (s, 1H).

MS (ESI⁺) m/z: 535 (MH⁺).

HRMS (ESI⁺) for C₃₀H₃₆FN₄O₄ (MH⁺): calcd, 535.27206. found, 535.27232.

### Step 3

### Benzyl (1-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropyl)methylcarbamate

The title compound benzyl (1-((7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropyl)methylcarbamate (84.0 mg) was prepared from benzyl (1-((8-(2-(4-amino-2-oxabicyclo[2.2.2]octan-1-yl)ethyl)-7-fluoro-1,5-naphthyridin-2-yloxy)methyl)cyclopropyl)methylcarbamate (80.0 mg) and I (28.0 mg) in the same manner as described for Step 3 of EXAMPLE 1.

¹H NMR (CDCl₃): δ 0.67 (s, 4H), 1.70-1.85 (m, 8H), 1.97-2.05 (m, 2H), 3.10-3.18 (m, 2H), 3.33 (d, J=5.5 Hz, 2H), 3.74 (s, 2H), 3.75 (s, 2H), 4.43 (s, 2H), 4.63 (s, 2H), 5.07 (s, 2H), 5.36 (br, 1H), 6.94 (d, J=7.9 Hz, 1H), 7.05 (d, J=9.2 Hz, 1H), 7.20 (d, J=7.9 Hz, 1H), 7.29-7.37 (m, 5H), 8.16 (d, J=9.2 Hz, 1H), 8.60 (s, 1H).

MS (ESI⁺) m/z: 697 (MH⁺).

HRMS (ESI⁺) for C₃₈H₄₂FN₆O₆ (MH⁺): calcd, 697.31498. found, 697.31473.

### Step 4

### 6-((1-(2-(6-((1-(Aminomethyl)cyclopropyl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

The title compound 6-((1-(2-(6-((1-(aminomethyl)cyclopropyl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one (35.1 mg) was prepared from benzyl (1-((7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropyl)methylcarbamate (75.0 mg) in the same manner as described for Step 4 of EXAMPLE 38.

¹H NMR (DMSO-d₆): δ 0.53 (d, J=12.8 Hz, 4H), 1.53-1.78 (m, 8H), 1.80-1.96 (m, 2H), 2.65 (s, 2H), 3.03-3.14 (m, 2H), 3.59 (s, 2H), 3.64 (s, 2H), 4.38 (s, 2H), 4.60 (s, 2H), 7.02 (d, J=7.9 Hz, 1H), 7.24 (d, J=9.7 Hz, 1H), 7.29 (d, J=8.6 Hz, 1H), 8.25 (d, J=8.6 Hz, 1H), 8.73 (s, 1H).

MS (ESI⁺) m/z: 563 (MH⁺).

HRMS (ESI⁺) for C₃₀H₃₆FN₆O₄ (MH⁺): calcd, 563.27821. found, 563.27849.

### Example 44

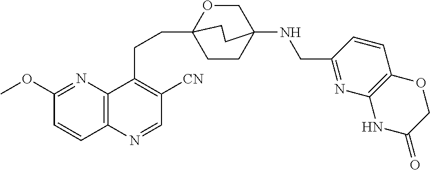 [SMILES: COc1ccc2ncc(C#N)c(CCC34CCC(NCc5ccc6c(n5)NC(=O)CO6)(CC3)CO4)c2n1]

### 6-Methoxy-4-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-3-carbonitrile

### Step 1

### tert-Butyl 1-(2-(3-Cyano-6-methoxy-1,5-naphthyridin-4-yl)vinyl)-2-oxabicyclo[2.2.2]octan-4-ylcarbamate

A mixture of Y (500 mg), B (690 mg), palladium(II) acetate (51.0 mg) and silver carbonate (377 mg) in benzene (15 mL) was heated under reflux for 2 days. After insoluble materials were filtered off, the filtrate was concentrated in vacuo. Flash chromatography (silica, hexane:ethyl acetate=2:1) of the residue gave tert-butyl 1-(2-(3-cyano-6-methoxy-1,5-naphthyridin-4-yl)vinyl)-2-oxabicyclo[2.2.2]octan-4-ylcarbamate (269 mg).

¹H NMR (CDCl₃): δ 1.45 (s, 9H), 1.94-2.05 (m, 4H), 2.12-2.20 (m, 4H), 4.10 (s, 5H), 4.35 (brs, 1H), 7.30 (d, J=9.2 Hz, 1H), 7.41 (d, J=16.6 Hz, 1H), 7.47 (d, J=16.5 Hz, 1H), 8.21 (d, J=9.2 Hz, 1H), 8.85 (s, 1H).

MS (ESI⁺) m/z: 437 (MH⁺).

HRMS (ESI⁺) for C₂₄H₂₉N₄O₄ (MH⁺): calcd, 437.21888. found, 437.21919.

### Step 2

### tert-Butyl 1-(2-(3-Cyano-6-methoxy-1,5-naphthyridin-4-yl)ethyl)-2-oxabicyclo[2.2.2]octan-4-ylcarbamate

The title compound tert-butyl 1-(2-(3-cyano-6-methoxy-1,5-naphthyridin-4-yl)ethyl)-2-oxabicyclo[2.2.2]octan-4-ylcarbamate (150 mg) was prepared from 1-(2-(3-cyano-6-methoxy-1,5-naphthyridin-4-yl)vinyl)-2-oxabicyclo[2.2.2]octan-4-ylcarbamate (264 mg) in the same manner as described for Step 2 of EXAMPLE 18.

¹H NMR (CDCl₃): δ 1.43 (s, 9H), 1.77-1.92 (m, 6H), 2.01-2.11 (m, 4H), 3.38-3.43 (m, 2H), 3.95 (s, 2H), 4.10 (s, 3H), 4.29 (brs, 1H), 7.22 (d, J=8.6 Hz, 1H), 8.20 (d, J=9.2 Hz, 1H), 8.81 (s, 1H).

MS (ESI⁺) m/z: 439 (MH⁺).

HRMS (ESI⁺) for C₂₄H₃₁N₄O₄ (MH⁺): calcd, 439.23453. found, 439.23489.

### Step 3

### 4-(2-(4-Amino-2-oxabicyclo[2.2.2]octan-1-yl)ethyl)-6-methoxy-1,5-naphthyridine-3-carbonitrile

The title compound 4-(2-(4-amino-2-oxabicyclo[2.2.2]octan-1-yl)ethyl)-6-methoxy-1,5-naphthyridine-3-carbonitrile (105 mg) was prepared from tert-butyl 1-(2-(3-cyano-6-methoxy-1,5-naphthyridin-4-yl)ethyl)-2-oxabicyclo[2.2.2]octan-4-ylcarbamate (143 mg) in the same manner as described for Step 2 of EXAMPLE 32.

¹H NMR (CDCl₃): δ 1.42 (s, 2H), 1.65-1.84 (m, 8H), 2.00-2.07 (m, 2H), 3.39-3.43 (m, 2H), 3.64 (s, 2H), 4.11 (s, 3H), 7.22 (d, J=8.6 Hz, 1H), 8.20 (d, J=9.2 Hz, 1H), 8.82 (s, 1H).

MS (ESI⁺) m/z: 339 (MH⁺).

HRMS (ESI⁺) for C₁₉H₂₃N₄O₂ (MH⁺): calcd, 339.18210. found, 339.18235.

### Step 4

### 6-Methoxy-4-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-3-carbonitrile

The title compound 6-methoxy-4-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-3-carbonitrile (122 mg) was prepared from 4-(2-(4-amino-2-oxabicyclo[2.2.2]octan-1-yl)ethyl)-6-methoxy-1,5-naphthyridine-3-carbonitrile (Example 44, Step 3, 101 mg) and I (49.0 mg) in the same manner as described for Step 3 of EXAMPLE 1.

¹H NMR (DMSO-d₆): δ 1.62-1.77 (m, 8H), 1.85-1.92 (m, 3H), 3.26-3.35 (m, 2H), 3.58 (s, 2H), 3.62 (d, J=4.9 Hz, 2H), 4.05 (s, 3H), 4.59 (s, 2H), 7.01 (d, J=7.9 Hz, 1H), 7.27 (d, J=7.9 Hz, 1H), 7.42 (d, J=9.2 Hz, 1H), 8.33 (d, J=8.6 Hz, 1H), 8.98 (s, 1H), 11.15 (brs, 1H).

MS (ESI⁺) m/z: 501 (MH⁺).

HRMS (ESI⁺) for C₂₇H₂₉N₆O₄ (MH⁺): calcd, 501.22503. found, 501.22516.

### Example 45

### 4-(2-(4-((2,3-Dihydro-[1,4]dioxino[2,3-c]pyridin-7-yl)methylamino)-2-oxabicyclo[2.2.2]octan-1-yl)ethyl)-6-methoxy-1,5-naphthyridine-3-carbonitrile Hydrochloride

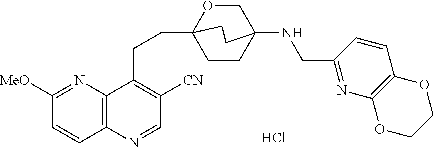 [SMILES: COc1ccc2ncc(C#N)c(CCC34CCC(NCc5ccc6c(n5)OCCO6)(CC3)CO4)c2n1 ; Cl]

### Step 1

### 4-(2-(4-((2,3-Dihydro-[1,4]dioxino[2,3-c]pyridin-7-yl)methylamino)-2-oxabicyclo[2.2.2]octan-1-yl)ethyl)-6-methoxy-1,5-naphthyridine-3-carbonitrile

The title compound 4-(2-(4-((2,3-dihydro-[1,4]dioxino[2,3-c]pyridin-7-yl)methylamino)-2-oxabicyclo[2.2.2]octan-1-yl)ethyl)-6-methoxy-1,5-naphthyridine-3-carbonitrile (116 mg) was prepared from 4-(2-(4-amino-2-oxabicyclo[2.2.2]octan-1-yl)ethyl)-6-methoxy-1,5-naphthyridine-3-carbonitrile (Example 44, Step 3, 93.0 mg) and 2,3-dihydro-[1,4]dioxino[2,3-c]pyridine-7-carbaldehyde (55.0 mg) in the same manner as described for Step 3 of EXAMPLE 1.

¹H NMR (CDCl₃): δ 1.76-1.84 (m, 9H), 2.00-2.08 (m, 2H), 3.74 (s, 2H), 3.75 (s, 2H), 4.11 (s, 3H), 4.26-4.28 (m, 2H), 4.31-4.33 (m, 2H), 6.82 (s, 1H), 7.22 (d, J=8.6 Hz, 1H), 8.09 (s, 1H), 8.20 (d, J=8.6 Hz, 1H), 8.81 (s, 1H).

MS (ESI⁺) m/z: 488 (MH⁺).

HRMS (ESI⁺) for C₂₇H₃₀N₅O₄ (MH⁺): calcd, 488.22978. found, 488.23043.

### Step 2

### 4-(2-(4-((2,3-Dihydro-[1,4]dioxino[2,3-c]pyridin-7-yl)methylamino)-2-oxabicyclo[2.2.2]octan-1-yl)ethyl)-6-methoxy-1,5-naphthyridine-3-carbonitrile Hydrochloride

The title compound 4-(2-(4-((2,3-dihydro-[1,4]dioxino[2,3-c]pyridin-7-yl)methylamino)-2-oxabicyclo[2.2.2]octan-1-yl)ethyl)-6-methoxy-1,5-naphthyridine-3-carbonitrile hydrochloride (106 mg) was prepared from 4-(2-(4-((2,3-dihydro-[1,4]dioxino[2,3-c]pyridin-7-yl)methylamino)-2-oxabicyclo[2.2.2]octan-1-yl)ethyl)-6-methoxy-1,5-naphthyridine-3-carbonitrile (110 mg) in the same manner as described for Step 4 of EXAMPLE 3.

¹H NMR (DMSO-d₆): δ 1.73-1.78 (m, 2H), 1.85-1.87 (m, 2H), 1.99-2.01 (m, 6H), 3.30-3.34 (m, 2H), 3.89 (s, 2H), 4.05 (s, 3H), 4.13 (s, 2H), 4.32-4.34 (m, 2H), 4.38-4.40 (m, 2H), 7.16 (s, 1H), 7.43 (d, J=9.2 Hz, 1H), 8.20 (s, 1H), 8.34 (d, J=9.2 Hz, 1H), 8.99 (s, 1H), 9.33 (brs, 2H).

MS (ESI⁺) m/z: 488 (MH⁺) (as free base).

HRMS (ESI⁺) for C₂₇H₃₀N₅O₄ (MH⁺) (as free base): calcd, 488.22978. found, 488.23060.

### Example 46

### 6-Methoxy-4-(2-(4-((1-methyl-2-oxo-1,2-dihydroquinolin-3-yl)methylamino)-2-oxabicyclo[2.2.2]octan-1-yl)ethyl)-1,5-naphthyridine-3-carbonitrile Hydrochloride

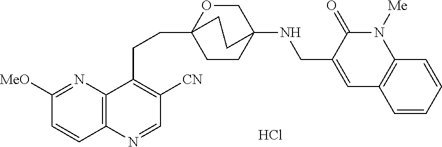 [SMILES: COc1ccc2ncc(C#N)c(CCC34CCC(NCc5cc6ccccc6n(C)c5=O)(CC3)CO4)c2n1 ; Cl]

### Step 1

### 6-Methoxy-4-(2-(4-((1-methyl-2-oxo-1,2-dihydroquinolin-3-yl)methylamino)-2-oxabicyclo[2.2.2]octan-1-yl)ethyl)-1,5-naphthyridine-3-carbonitrile

The title compound 6-methoxy-4-(2-(4-((1-methyl-2-oxo-1,2-dihydroquinolin-3-yl)methylamino)-2-oxabicyclo[2.2.2]octan-1-yl)ethyl)-1,5-naphthyridine-3-carbonitrile (113 mg) was prepared from 4-(2-(4-amino-2-oxabicyclo[2.2.2]octan-1-yl)ethyl)-6-methoxy-1,5-naphthyridine-3-carbonitrile (Example 44, Step 3, 94.0 mg) and 1-methyl-2-oxo-1,2-dihydroquinoline-3-carbaldehyde (63.0 mg) in the same manner as described for Step 3 of EXAMPLE 1.

¹H NMR (CDCl₃): δ 1.81-1.90 (m, 9H), 2.05-2.07 (m, 2H), 3.41-3.45 (m, 2H), 3.74 (s, 5H), 3.81 (s, 2H), 4.11 (s, 3H), 7.21-7.24 (m, 2H), 7.36 (d, J=8.6 Hz, 1H), 7.52-7.58 (m, 2H), 7.74 (s, 1H), 8.20 (d, J=9.2 Hz, 1H), 8.81 (s, 1H).

MS (ESI⁺) m/z: 510 (MH⁺).

HRMS (ESI⁺) for C₃₀H₃₂N₅O₃ (MH⁺): calcd, 510.25051. found, 510.24993.

### Step 2

### 6-Methoxy-4-(2-(4-((1-methyl-2-oxo-1,2-dihydroquinolin-3-yl)methylamino)-2-oxabicyclo[2.2.2]octan-1-yl)ethyl)-1,5-naphthyridine-3-carbonitrile Hydrochloride

The title compound 6-methoxy-4-(2-(4-((1-methyl-2-oxo-1,2-dihydroquinolin-3-yl)methylamino)-2-oxabicyclo[2.2.2]octan-1-yl)ethyl)-1,5-naphthyridine-3-carbonitrile hydrochloride (106 mg) was prepared from 6-methoxy-4-(2-(4-((1-methyl-2-oxo-1,2-dihydroquinolin-3-yl)methylamino)-2-oxabicyclo[2.2.2]octan-1-yl)ethyl)-1,5-naphthyridine-3-carbonitrile (107 mg) in the same manner as described for Step 4 of EXAMPLE 3.

¹H NMR (DMSO-d₆): δ 1.75-1.80 (m, 2H), 1.87-1.90 (m, 2H), 2.02-2.05 (m, 6H), 3.31-3.36 (m, 2H), 3.71 (s, 3H), 3.93 (s, 2H), 4.07 (s, 5H), 7.35 (t, J=7.9 Hz, 1H), 7.44 (d, J=9.2 Hz, 1H), 7.62 (d, J=8.6 Hz, 1H), 7.71 (t, J=8.6 Hz, 1H), 7.79 (d, J=6.1 Hz, 1H), 8.20 (s, 1H), 8.35 (d, J=9.2 Hz, 1H), 9.00 (s, 1H), 9.02 (brs, 2H).

MS (ESI⁺) m/z: 510 (MH⁺) (as free base).

HRMS (ESI⁺) for C₃₀H₃₂N₅O₃ (MH⁺) (as free base): calcd, 510.25051. found, 510.25130.

### Example 47

### 6-((1-(2-(3-Fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one O-Methyl Oxime

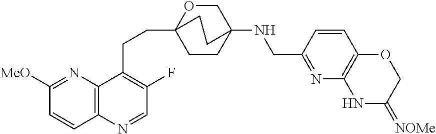

To a solution of 6-((1-(2-(3-fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one (Example 18, Step 4, 200 mg) in pyridine (10.1 mL) was added O-methylhydroxylamine hydrochloride (542 mg), the mixture was stirred at 80° C. for 72 hours and then concentrated in vacuo. After dilution of the residue with dichloromethane, the mixture was washed with water, saturated sodium hydrogencarbonate solution and brine, dried over anhydrous sodium sulfate, filtered, and then concentrated in vacuo. Preparative thin layer chromatography (silica, chloroform:methanol=10:1) of the residue gave 6-((1-(2-(3-fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one O-methyl oxime (130 mg).

¹H NMR (DMSO-d₆): δ 1.58-1.77 (m, 8H), 1.81-1.95 (m, 3H), 3.07-3.15 (m, 2H), 3.58 (s, 2H), 3.61 (s, 2H), 3.71 (s, 3H), 4.03 (s, 3H), 4.51 (s, 2H), 6.89 (d, J=7.9 Hz, 1H), 7.20 (d, J=7.9 Hz, 1H), 7.22 (d, J=9.1 Hz, 1H), 8.26 (d, J=9.1 Hz, 1H), 8.74 (s, 1H), 9.76 (s, 1H).

MS (ESI⁺) m/z: 523 (MH⁺).

HRMS (ESI⁺) for C₂₇H₃₂FN₆O₄ (MH⁺): calcd, 523.24691. found, 523.24743.

### Example 48

### 6-((1-(2-(3-Fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one Oxime

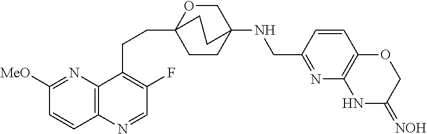

The title compound 6-((1-(2-(3-fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one oxime (24.2 mg) was prepared from 6-((1-(2-(3-fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one (Example 18, Step 4, 45.0 mg) and hydroxylamine hydrochloride (101 mg) in the same manner as described for EXAMPLE 47.

¹H NMR (DMSO-d₆): δ 1.59-1.77 (m, 8H), 1.81-1.93 (m, 3H), 3.08-3.15 (m, 2H), 3.58 (s, 2H), 3.59 (brs, 2H), 4.03 (s, 3H), 4.51 (s, 2H), 6.58 (d, J=8.5 Hz, 1H), 7.17 (d, J=7.9 Hz, 1H), 7.22 (d, J=9.1 Hz, 1H), 8.26 (d, J=9.1 Hz, 1H), 8.74 (s, 1H), 9.35 (s, 1H) 10.03 (s, 1H).

MS (ESI⁺) m/z: 509 (MH⁺).

HRMS (ESI⁺) for C₂₆H₃₀FN₆O₄ (MH⁺): calcd, 509.23126. found, 509.23039.

### Example 49

### 6-(((1-(2-(6-Methoxy-1-methyl-2-oxo-1,2-dihydro-1,5-naphthyridin-4-yl)ethyl)-2-oxabicyclo[2.2.2]octan-4-yl)amino)methyl)-2H-pyrido[3,2-b][1,4]oxazin-3(4H)-one

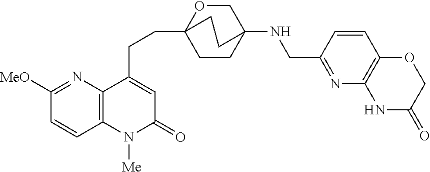 [SMILES: COc1ccc2c(n1)c(CCC13CCC(NCc4ccc5c(n4)NC(=O)CO5)(CC1)CO3)cc(=O)n2C]

### Step 1

### 4-(2-(4-((tert-Butoxycarbonyl)amino)-2-oxabicyclo[2.2.2]octan-1-yl)ethyl)-6-methoxy-1-methyl-1,5-naphthyridin-1-ium Trifluoromethanesulfonate

To a solution of Z (450 mg) in dichloromethane (10.9 mL) was added methyl trifluoromethanesulfonate (135 μL) under cooling with ice, the mixture was stirred at room temperature for 4 hours and concentrated in vacuo. Treatment of the residue with diethyl ether gave 4-(2-(4-((tert-butoxycarbonyl)amino)-2-oxabicyclo[2.2.2]octan-1-yl)ethyl)-6-methoxy-1-methyl-1,5-naphthyridin-1-ium trifluoromethanesulfonate (587 mg).

¹H NMR (DMSO-d₆): δ 1.35 (s, 9H), 1.69-1.96 (m, 10H), 3.29-3.33 (m, 2H), 3.76 (s, 2H), 4.11 (s, 3H), 4.52 (s, 3H), 6.60 (br, 1H), 7.75 (d, J=9.2 Hz, 1H), 8.19 (d, J=6.1 Hz, 1H), 8.78 (d, J=9.8 Hz, 1H), 9.17 (d, J=6.1 Hz, 1H).

MS (ESI⁺) m/z: 428 [(M—CF₃SO₃)⁺].

HRMS (ESI⁺) for C₂₄H₃₄N₃O₄ [(M—CF₃SO₃)⁺]: calcd, 428.25493. found, 428.25409.

### Step 2

### tert-Butyl (1-(2-(6-Methoxy-1-methyl-2-oxo-1,2-dihydro-1,5-naphthyridin-4-yl)ethyl)-2-oxabicyclo[2.2.2]octan-4-yl)carbamate

To a suspension of 4-(2-(4-((tert-butoxycarbonyl)amino)-2-oxabicyclo[2.2.2]octan-1-yl)ethyl)-6-methoxy-1-methyl-1,5-naphthyridin-1-ium trifluoromethanesulfonate (300 mg) in tetrahydrofuran (10.4 mL) were added potassium ferricyanide (1.76 g) and 1 M sodium hydroxide solution (10.5 mL) under cooling with ice, the mixture was stirred at the same temperature for 15 minutes. After dilution of the mixture with dichloromethane, the mixture was washed with water. The organic extracts were washed with brine, dried over anhydrous sodium sulfate, filtered, and then concentrated in vacuo. Flash chromatography (silica, dichloromethane:ethyl acetate=1:1) of the residue gave tert-butyl (1-(2-(6-methoxy-1-methyl-2-oxo-1,2-dihydro-1,5-naphthyridin-4-yl)ethyl)-2-oxabicyclo[2.2.2]octan-4-yl)carbamate (64.0 mg).

¹H NMR (CDCl₃): δ 1.43 (s, 9H), 1.69-1.79 (m, 4H), 1.83-1.91 (m, 2H), 1.95-2.17 (m, 4H), 2.91-2.98 (m, 2H), 3.67 (s, 3H), 3.97 (s, 2H), 3.99 (s, 3H), 4.29 (brs, 1H), 6.75 (s, 1H), 6.96 (d, J=9.2 Hz, 1H), 7.62 (d, J=9.2 Hz, 1H).

MS (CI⁺) m/z: 444 (MH⁺).

HRMS (CI⁺) for C₂₄H₃₄N₃O₅ (MH⁺): calcd, 444.2498. found, 444.2488.

### Step 3

### 4-(2-(4-Amino-2-oxabicyclo[2.2.2]octan-1-yl)ethyl)-6-methoxy-1-methyl-1,5-naphthyridin-2(1H)-one

The title compound 4-(2-(4-amino-2-oxabicyclo[2.2.2]octan-1-yl)ethyl)-6-methoxy-1-methyl-1,5-naphthyridin-2(1H)-one (31.2 mg) was prepared from tert-butyl (1-(2-(6-methoxy-1-methyl-2-oxo-1,2-dihydro-1,5-naphthyridin-4-yl)ethyl)-2-oxabicyclo[2.2.2]octan-4-yl)carbamate (45.0 mg) in the same manner as described for Step 2 of EXAMPLE 1.

¹H NMR (CDCl₃): δ 1.63-1.80 (m, 8H), 1.92-2.02 (m, 2H), 2.91-2.99 (m, 2H), 3.65 (s, 2H), 3.68 (s, 3H), 4.01 (s, 3H), 6.76 (s, 1H), 6.96 (d, J=9.2 Hz, 1H), 7.62 (d, J=9.2 Hz, 1H).

MS (ESI⁺) m/z: 344 (MH⁺).

HRMS (ESI⁺) for C₁₉H₂₆N₃O₃ (MH⁺): calcd, 344.19742. found, 344.19807.

### Step 4

### 6-(((1-(2-(6-Methoxy-1-methyl-2-oxo-1,2-dihydro-1,5-naphthyridin-4-yl)ethyl)-2-oxabicyclo[2.2.2]octan-4-yl)amino)methyl)-2H-pyrido[3,2-b][1,4]oxazin-3(4H)-one

The title compound 6-(((1-(2-(6-methoxy-1-methyl-2-oxo-1,2-dihydro-1,5-naphthyridin-4-yl)ethyl)-2-oxabicyclo[2.2.2]octan-4-yl)amino)methyl)-2H-pyrido[3,2-b][1,4]oxazin-3(4H)-one (36.4 mg) was prepared from 4-(2-(4-amino-2-oxabicyclo[2.2.2]octan-1-yl)ethyl)-6-methoxy-1-methyl-1,5-naphthyridin-2(1H)-one (30.0 mg) and I (16.3 mg) in the same manner as described for Step 3 of EXAMPLE 1.

¹H NMR (CDCl₃): δ 1.57-1.75 (m, 8H), 1.78-1.90 (m, 3H), 2.83-2.87 (m, 2H), 3.56 (s, 3H), 3.57 (s, 2H), 3.62 (s, 2H), 3.91 (s, 3H), 4.59 (s, 2H), 6.64 (s, 1H), 7.00 (d, J=7.9 Hz, 1H), 7.10 (d, J=9.2 Hz, 1H), 7.27 (d, J=7.9 Hz, 1H), 7.95 (d, J=9.2 Hz, 1H), 11.15 (brs, 1H).

MS (ESI⁺) m/z: 506 (MH⁺).

HRMS (ESI⁺) for C₂₇H₃₂N₅O₅ (MH⁺): calcd, 506.24034. found, 506.24058.

### Example 50

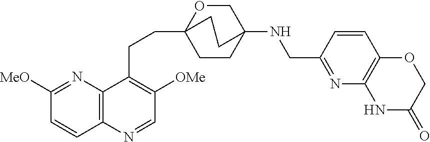 [SMILES: COc1ccc2ncc(OC)c(CCC34CCC(NCc5ccc6c(n5)NC(=O)CO6)(CC3)CO4)c2n1]

### 6-[({1-[2-(3,6-Dimethoxy-1,5-naphthyridin-4-yl)ethyl]-2-oxabicyclo[2.2.2]oct-4-yl}amino)methyl]-2H-pyrido[3,2-b][1,4]oxazin-3(4H)-one

### Step 1

### 1-(2-(3,6-Dimethoxy-1,5-naphthyridin-4-yl)ethyl)-2-oxabicyclo[2.2.2]octan-4-amine

To a solution of tert-butyl 1-(2-(3-fluoro-6-methoxy-1,5-naphthyridin-4-yl)ethyl)-2-oxabicyclo[2.2.2]octan-4-ylcarbamate (Example 18, Step 2, 30.0 mg) in methanol (0.17 mL) was added a solution of sodium methoxide (150 μL, 25 wt % in methanol), the mixture was stirred under reflux for 24 hours. After dilution of the mixture with dichloromethane, the mixture was washed with water. The organic extracts were washed with brine, dried over anhydrous sodium sulfate, filtered, and then concentrated in vacuo to give 1-(2-(3,6-dimethoxy-1,5-naphthyridin-4-yl)ethyl)-2-oxabicyclo[2.2.2]octan-4-amine (21.3 mg).

¹H NMR (DMSO-d₆): δ 1.22 (s, 2H), 1.50-1.97 (m, 10H), 3.03-3.15 (m, 2H), 3.44 (s, 2H), 4.02 (s, 3H), 4.07 (s, 3H), 7.06 (dd, J=8.9, 1.5 Hz, 1H), 8.15 (dd, J=9.2, 1.2 Hz, 1H), 8.68 (d, J=1.2 Hz, 1H).

### Step 2

### 6-(((1-(2-(3,6-Dimethoxy-1,5-naphthyridin-4-yl)ethyl)-2-oxabicyclo[2.2.2]octan-4-yl)amino)methyl)-2H-pyrido[3,2-b][1,4]oxazin-3(4H)-one

The title compound 6-(((1-(2-(3,6-dimethoxy-1,5-naphthyridin-4-yl)ethyl)-2-oxabicyclo[2.2.2]octan-4-yl)amino)methyl)-2H-pyrido[3,2-b][1,4]oxazin-3(4H)-one (12.8 mg) was prepared from 1-(2-(3,6-dimethoxy-1,5-naphthyridin-4-yl)ethyl)-2-oxabicyclo[2.2.2]octan-4-amine (18.2 mg) and I (9.90 mg) in the same manner as described for Step 3 of EXAMPLE 1.

¹H NMR (CDCl₃): δ 1.68-1.72 (m, 2H), 1.80-1.87 (m, 6H), 2.02-2.17 (m, 2H), 3.16-3.20 (m, 2H), 3.76 (s, 2H), 3.79 (s, 2H), 4.05 (s, 3H), 4.07 (s, 3H), 4.63 (s, 2H), 6.94 (d, J=7.9 Hz, 1H), 6.96 (d, J=8.6 Hz, 1H), 7.20 (d, J=7.9 Hz, 1H), 8.05 (br, 2H), 8.11 (d, J=9.2 Hz, 1H), 8.56 (s, 1H).

MS (EI⁺) m/z: 505 (M⁺).

HRMS (EI⁺) for C₂₇H₃₁N₅O₅ (M⁺): calcd, 505.2325. found, 505.2306.

### Example 51

### 6-(((1-(1-Hydroxy-2-(6-methoxy-3-oxopyrido[2,3-b]pyrazin-4(3H)-yl)ethyl)-2-oxabicyclo[2.2.2]octan-4-yl)amino)methyl)-2H-pyrido[3,2-b][1,4]oxazin-3(4H)-one Hydrochloride

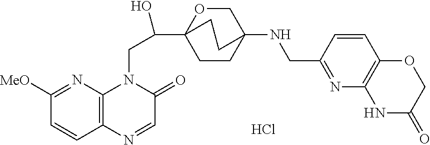 [SMILES: COc1ccc2ncc(=O)n(CC(O)C34CCC(NCc5ccc6c(n5)NC(=O)CO6)(CC3)CO4)c2n1 ; Cl]

### Step 1

### tert-Butyl (1-(1-Hydroxy-2-(6-methoxy-3-oxopyrido[2,3-b]pyrazin-4(3H)-yl)ethyl)-2-oxabicyclo[2.2.2]octan-4-yl)carbamate (Enantiomer A and Enantiomer B)

A mixture of 6-methoxypyrido[3,2-b]pyrazin-3(4H)-one (250 mg), AA (418 mg) and cesium carbonate (920 mg) in N,N-dimethylformamide (4.9 mL) was stirred at 75° C. for 22 hours, and then concentrated in vacuo. Flash chromatography (silica, hexane:ethyl acetate=5:2) of the residue gave tert-butyl (1-(1-hydroxy-2-(6-methoxy-3-oxopyrido[2,3-b]pyrazin-4(3H)-yl)ethyl)-2-oxabicyclo[2.2.2]octan-4-yl)carbamate (284 mg).

Optical resolution (CHIRALPAK IA, hexane:ethanol=30:70) of the racemate (220 mg) gave Enantiomer A (90.0 mg) and Enantiomer B (91.0 mg).

Enantiomer A: ¹H NMR (CDCl₃): δ 1.45 (s, 9H), 1.79-2.22 (m, 8H), 3.11 (d, J=6.7 Hz, 1H), 3.80-3.85 (m, 1H), 3.99-4.06 (m, 5H), 4.34 (s, 1H), 4.61 (dd, J=13.4, 9.8 Hz, 1H), 4.77 (dd, J=13.1, 2.8 Hz, 1H), 6.75 (d, J=8.6 Hz, 1H), 8.04 (d, J=9.2 Hz, 1H), 8.20 (s, 1H).

MS (ESI⁺) m/z: 447 (MH⁺).

HRMS (ESI⁺) for C₂₂H₃₁N₄O₆ (MH⁺): calcd, 447.22436. found, 447.22457.

Enantiomer B: ¹H NMR (CDCl₃): δ 1.43 (s, 9H), 1.78-2.20 (m, 8H), 3.09 (d, J=6.7 Hz, 1H), 3.49 (d, J=5.5 Hz, 1H), 3.83-3.79 (m, 1H), 3.99-4.06 (m, 5H), 4.32 (s, 1H), 4.59 (dd, J=12.8, 9.8 Hz, 1H), 4.76 (dd, J=12.8, 2.4 Hz, 1H), 6.74 (d, J=8.6 Hz, 1H), 8.03 (d, J=8.6 Hz, 1H), 8.18 (s, 1H).

MS (ESI⁺) m/z: 447 (MH⁺).

HRMS (ESI⁺) for C₂₂H₃₁N₄O₆ (MH⁺): calcd, 447.22436. found, 447.22435.

### Step 2

### 4-(2-(4-Amino-2-oxabicyclo[2.2.2]octan-1-yl)-2-hydroxyethyl)-6-methoxypyrido[2,3-b]pyrazin-3(4H)-one (Enantiomer A)

The title compound 4-(2-(4-amino-2-oxabicyclo[2.2.2]octan-1-yl)-2-hydroxyethyl)-6-methoxypyrido[2,3-b]pyrazin-3(4H)-one (48.4 mg) was prepared from tert-butyl (1-(1-hydroxy-2-(6-methoxy-3-oxopyrido[2,3-b]pyrazin-4(3H)-yl)ethyl)-2-oxabicyclo[2.2.2]octan-4-yl)carbamate (67.2 mg) in the same manner as described for Step 2 of EXAMPLE 32.

¹H NMR (CDCl₃): δ 1.64-2.21 (m, 10H), 3.67 (s, 2H), 3.83 (dd, J=9.8, 2.4 Hz, 1H), 4.03 (s, 3H), 4.62 (dd, J=13.4, 9.8 Hz, 1H), 4.72 (dd, J=13.1, 2.8 Hz, 1H), 6.74 (d, J=8.6 Hz, 1H), 8.03 (d, J=8.6 Hz, 1H), 8.19 (s, 1H).

MS (ESI⁺) m/z: 347 (MH⁺).

HRMS (ESI⁺) for C₁₇H₂₃N₄O₄ (MH⁺): calcd, 347.17193. found, 347.17192.

Enantiomer B of 4-(2-(4-amino-2-oxabicyclo[2.2.2]octan-1-yl)-2-hydroxyethyl)-6-methoxypyrido[2,3-b]pyrazin-3(4H)-one (38.2 mg) was prepared in the same manner from tert-butyl (1-(1-hydroxy-2-(6-methoxy-3-oxopyrido[2,3-b]pyrazin-4(3H)-yl)ethyl)-2-oxabicyclo[2.2.2]octan-4-yl)carbamate (53.0 mg, Enantiomer B).

¹H NMR (CDCl₃): δ 1.65-2.21 (m, 10H), 3.67 (s, 2H), 3.83 (dd, J=9.8, 2.4 Hz, 1H), 4.03 (s, 3H), 4.62 (dd, J=13.4, 9.8 Hz, 1H), 4.72 (dd, J=13.1, 2.8 Hz, 1H), 6.74 (d, J=8.6 Hz, 1H), 8.03 (d, J=8.6 Hz, 1H), 8.19 (s, 1H).

MS (ESI⁺) m/z: 347 (MH⁺).

HRMS (ESI⁺) for C₁₇H₂₃N₄O₄ (MH⁺): calcd, 347.17193. found, 347.17185.

### Step 3

### 6-(((1-(1-Hydroxy-2-(6-methoxy-3-oxopyrido[2,3-b]pyrazin-4(3H)-yl)ethyl)-2-oxabicyclo[2.2.2]octan-4-yl)amino)methyl)-2H-pyrido[3,2-b][1,4]oxazin-3(4H)-one Hydrochloride (Enantiomer A)

The compound 6-(((1-(1-hydroxy-2-(6-methoxy-3-oxopyrido[2,3-b]pyrazin-4(3H)-yl)ethyl)-2-oxabicyclo[2.2.2]octan-4-yl)amino)methyl)-2H-pyrido[3,2-b][1,4]oxazin-3(4H)-one (51.0 mg) was prepared from 4-(2-(4-amino-2-oxabicyclo[2.2.2]octan-1-yl)-2-hydroxyethyl)-6-methoxypyrido[2,3-b]pyrazin-3(4H)-one (45.0 mg, Enantiomer A) and I (24.3 mg) in the same manner as described for Step 3 of EXAMPLE 1. The title compound 6-(((1-(1-hydroxy-2-(6-methoxy-3-oxopyrido[2,3-b]pyrazin-4(3H)-yl)ethyl)-2-oxabicyclo[2.2.2]octan-4-yl)amino)methyl)-2H-pyrido[3,2-b][1,4]oxazin-3(4H)-one hydrochloride (24.2 mg, Enantiomer A) was prepared from 6-(((1-(1-hydroxy-2-(6-methoxy-3-oxopyrido[2,3-b]pyrazin-4(3H)-yl)ethyl)-2-oxabicyclo[2.2.2]octan-4-yl)amino)methyl)-2H-pyrido[3,2-b][1,4]oxazin-3(4H)-one (37.0 mg, Enantiomer A) in the same manner as described for Step 4 of EXAMPLE 3.

¹H NMR (DMSO-d₆): δ 1.85-2.12 (m, 8H), 3.88 (brs, 3H), 3.98 (s, 3H), 4.10 (brs, 2H), 4.22 (dd, J=12.8, 3.7 Hz, 1H), 4.65 (dd, J=12.8, 9.8 Hz, 1H), 4.68 (s, 2H), 4.84 (d, J=5.5 Hz, 1H), 6.82 (d, J=8.6 Hz, 1H), 7.19 (d, J=8.6 Hz, 1H), 7.45 (d, J=8.6 Hz, 1H), 8.10 (s, 1H), 8.12 (d, J=8.6 Hz, 1H), 9.24 (brs, 2H), 11.32 (s, 1H).

MS (ESI⁺) m/z: 509 (MH⁺) (as free base).

HRMS (ESI⁺) for C₂₅H₂₉N₆O₆ (MH⁺) (as free base): calcd, 509.21486. found, 509.21393.

Enantiomer B of 6-(((1-(1-hydroxy-2-(6-methoxy-3-oxopyrido[2,3-b]pyrazin-4(3H)-yl)ethyl)-2-oxabicyclo[2.2.2]octan-4-yl)amino)methyl)-2H-pyrido[3,2-b][1,4]oxazin-3(4H)-one (44.0 mg) was prepared in the same manner from 4-(2-(4-amino-2-oxabicyclo[2.2.2]octan-1-yl)-2-hydroxyethyl)-6-methoxypyrido[2,3-b]pyrazin-3(4H)-one (37.0 mg, Enantiomer B) and I (20.0 mg). Enantiomer B of 6-(((1-(1-hydroxy-2-(6-methoxy-3-oxopyrido[2,3-b]pyrazin-4(3H)-yl)ethyl)-2-oxabicyclo[2.2.2]octan-4-yl)amino)methyl)-2H-pyrido[3,2-b][1,4]oxazin-3(4H)-one hydrochloride (28.0 mg) was prepared in the same manner from 6-(((1-(1-hydroxy-2-(6-methoxy-3-oxopyrido[2,3-b]pyrazin-4(3H)-yl)ethyl)-2-oxabicyclo[2.2.2]octan-4-yl)amino)methyl)-2H-pyrido[3,2-b][1,4]oxazin-3(4H)-one (42.0 mg, Enantiomer B).

¹H NMR (DMSO-d₆): δ 1.85-2.12 (m, 8H), 3.88 (brs, 3H), 3.98 (s, 3H), 4.10 (brs, 2H), 4.22 (dd, J=12.8, 3.7 Hz, 1H), 4.65 (dd, J=12.8, 9.8 Hz, 1H), 4.69 (s, 2H), 4.84 (d, J=6.1 Hz, 1H), 6.82 (d, J=8.6 Hz, 1H), 7.19 (d, J=7.9 Hz, 1H), 7.45 (d, J=7.9 Hz, 1H), 8.10 (s, 1H), 8.12 (d, J=8.6 Hz, 1H), 9.23 (brs, 2H), 11.32 (s, 1H).

MS (ESI⁺) m/z: 509 (MH⁺) (as free base).

HRMS (ESI⁺) for C₂₅H₂₉N₆O₆ (MH⁺) (as free base): calcd, 509.21486. found, 509.21421.

### Example 52

### 6-(((1-(1-Hydroxy-2-(7-methoxy-2-oxo-1,8-naphthyridin-1(2H)-yl)ethyl)-2-oxabicyclo[2.2.2]octan-4-yl)amino)methyl)-2H-pyrido[3,2-b][1,4]oxazin-3(4H)-one (Enantiomer A and Enantiomer B)

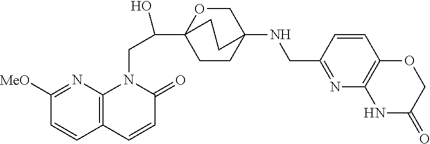 [SMILES: COc1ccc2ccc(=O)n(CC(O)C34CCC(NCc5ccc6c(n5)NC(=O)CO6)(CC3)CO4)c2n1]

### Step 1

### tert-Butyl (1-(1-Hydroxy-2-(7-methoxy-2-oxo-1,8-naphthyridin-1(2H)-yl)ethyl)-2-oxabicyclo[2.2.2]octan-4-yl)carbamate (Enantiomer A and Enantiomer B)

The title compound (1-(1-hydroxy-2-(7-methoxy-2-oxo-1,8-naphthyridin-1(2H)-yl)ethyl)-2-oxabicyclo[2.2.2]octan-4-yl)carbamate (171 mg) was prepared from 7-methoxy-1,8-naphthyridin-2(1H)-one (100 mg) and AA (168.2 mg) in the same manner as described for Step 1 of EXAMPLE 51.

¹H NMR (CDCl₃): δ 1.43 (s, 9H), 1.84-2.17 (m, 10H), 3.75-3.79 (m, 1H), 3.96 (d, J=4.9 Hz, 1H), 4.01 (s, 2H), 4.03 (s, 3H), 4.32 (s, 1H), 4.52 (dd, J=13.4, 9.8 Hz, 1H), 5.03 (dd, J=13.1, 2.1 Hz, 1H), 6.62 (d, J=9.2 Hz, 1H), 6.64 (d, J=8.6 Hz, 1H), 7.61 (d, J=9.2 Hz, 1H), 7.74 (d, J=8.6 Hz, 1H).

Optical resolution (CHIRALPAK IA, hexane:ethanol=30:70) of the racemate (220 mg) gave Enantiomer A (90.0 mg) and Enantiomer B (91.0 mg).

### Step 2

### 1-(2-(4-Amino-2-oxabicyclo[2.2.2]octan-1-yl)-2-hydroxyethyl)-7-methoxy-1,8-naphthyridin-2(1H)-one (Enantiomer A)

The title compound 1-(2-(4-amino-2-oxabicyclo[2.2.2]octan-1-yl)-2-hydroxyethyl)-7-methoxy-1,8-naphthyridin-2(1H)-one (80.1 mg) was prepared from tert-butyl (1-(1-hydroxy-2-(7-methoxy-2-oxo-1,8-naphthyridin-1(2H)-yl)ethyl)-2-oxabicyclo[2.2.2]octan-4-yl)carbamate (113 mg, Enantiomer A) in the same manner as described for Step 2 of EXAMPLE 32.

¹H NMR (DMSO-d₆): δ 1.65-2.17 (m, 10H), 3.67 (s, 2H), 3.80 (dd, J=9.8, 2.4 Hz, 1H), 4.03 (s, 3H), 4.56 (dd, J=13.4, 9.8 Hz, 1H), 4.99 (dd, J=13.4, 2.4 Hz, 1H), 6.62 (d, J=9.2 Hz, 1H), 6.64 (d, J=8.6 Hz, 1H), 7.61 (d, J=9.2 Hz, 1H), 7.75 (d, J=8.6 Hz, 1H).

MS (ESI⁺) m/z: 346 (MH⁺).

HRMS (ESI⁺) for C₁₈H₂₄N₃O₄ (MH⁺): calcd, 346.17668. found, 346.17700.

Enantiomer B of 1-(2-(4-amino-2-oxabicyclo[2.2.2]octan-1-yl)-2-hydroxyethyl)-7-methoxy-1,8-naphthyridin-2(1H)-one (82.7 mg) was prepared in the same manner from tert-butyl (1-(1-hydroxy-2-(7-methoxy-2-oxo-1,8-naphthyridin-1(2H)-yl)ethyl)-2-oxabicyclo[2.2.2]octan-4-yl)carbamate (111 mg, Enantiomer B).

MS (ESI⁺) m/z: 346 (MH⁺).

HRMS (ESI⁺) for C₁₈H₂₄N₃O₄ (MH⁺): calcd, 346.17668. found, 346.17745.

### Step 3

### 6-(((1-(1-Hydroxy-2-(7-methoxy-2-oxo-1,8-naphthyridin-1(2H)-yl)ethyl)-2-oxabicyclo[2.2.2]octan-4-yl)amino)methyl)-2H-pyrido[3,2-b][1,4]oxazin-3(4H)-one (Enantiomer A)

The title compound 6-(((1-(1-hydroxy-2-(7-methoxy-2-oxo-1,8-naphthyridin-1(2H)-yl)ethyl)-2-oxabicyclo[2.2.2]octan-4-yl)amino)methyl)-2H-pyrido[3,2-b][1,4]oxazin-3(4H)-one (84.2 mg) was prepared from 1-(2-(4-amino-2-oxabicyclo[2.2.2]octan-1-yl)-2-hydroxyethyl)-7-methoxy-1,8-naphthyridin-2(1H)-one (75.0 mg, Enantiomer A) and I (40.6 mg) in the same manner as described for Step 3 of EXAMPLE 1.

¹H NMR (DMSO-d₆): δ 1.61-1.98 (m, 9H), 3.56 (s, 2H), 3.62 (s, 2H), 3.78-3.82 (m, 1H), 3.95 (s, 3H), 4.29-4.33 (m, 2H), 4.59 (s, 2H), 4.70 (dd, J=13.4, 9.8 Hz, 1H), 6.47 (d, J=9.2 Hz, 1H), 6.69 (d, J=7.9 Hz, 1H), 7.00 (d, J=7.9 Hz, 1H), 7.27 (d, J=7.9 Hz, 1H), 7.83 (d, J=9.2 Hz, 1H), 8.01 (d, J=8.6 Hz, 1H), 11.15 (s, 1H).

MS (ESI⁺) m/z: 508 (MH⁺).

HRMS (ESI⁺) for C₂₆H₃₀N₅O₆ (MH⁺): calcd, 508.21961. found, 508.21998.

Enantiomer B of 6-(((1-(1-hydroxy-2-(7-methoxy-2-oxo-1,8-naphthyridin-1(2H)-yl)ethyl)-2-oxabicyclo[2.2.2]octan-4-yl)amino)methyl)-2H-pyrido[3,2-b][1,4]oxazin-3(4H)-one (72.6 mg) was prepared in the same manner from 1-(2-(4-amino-2-oxabicyclo[2.2.2]octan-1-yl)-2-hydroxyethyl)-7-methoxy-1,8-naphthyridin-2(1H)-one (75.0 mg, Enantiomer B) and I (40.6 mg).

¹H NMR (DMSO-d₆): δ 1.61-1.98 (m, 9H), 3.56 (s, 2H), 3.62 (s, 2H), 3.78-3.83 (m, 1H), 3.95 (s, 3H), 4.30-4.33 (m, 2H), 4.59 (s, 2H), 4.70 (dd, J=13.4, 9.8 Hz, 1H), 6.47 (d, J=9.2 Hz, 1H), 6.69 (d, J=8.6 Hz, 1H), 7.00 (d, J=7.9 Hz, 1H), 7.27 (d, J=8.6 Hz, 1H), 7.83 (d, J=9.2 Hz, 1H), 8.01 (d, J=8.6 Hz, 1H), 11.15 (s, 1H).

MS (ESI⁺) m/z: 508 (MH⁺).

HRMS (ESI⁺) for C₂₆H₃₀N₅O₆ (MH⁺): calcd, 508.21961. found, 508.22039.

### Example 53

### 6-(((1-(1-Hydroxy-2-(7-methyl-2-oxo-1,8-naphthyridin-1(2H)-yl)ethyl)-2-oxabicyclo[2.2.2]octan-4-yl)amino)methyl)-2H-pyrido[3,2-b][1,4]oxazin-3(4H)-one (Enantiomer A and Enantiomer B)

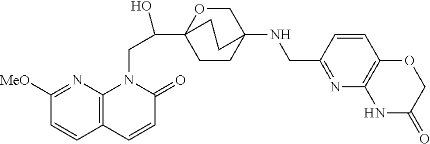 [SMILES: COc1ccc2ccc(=O)n(CC(O)C34CCC(NCc5ccc6c(n5)NC(=O)CO6)(CC3)CO4)c2n1]

### Step 1

### tert-Butyl (1-(1-Hydroxy-2-(7-methyl-2-oxo-1,8-naphthyridin-1(2H)-yl)ethyl)-2-oxabicyclo[2.2.2]octan-4-yl)carbamate (Enantiomer A)

The title compound tert-butyl (1-(1-hydroxy-2-(7-methyl-2-oxo-1,8-naphthyridin-1(2H)-yl)ethyl)-2-oxabicyclo[2.2.2]octan-4-yl)carbamate (64.2 mg) was prepared from 7-methyl-1,8-naphthyridin-2(1H)-one (60.0 mg) and AB (165 mg, Enantiomer A) in the same manner as described for EXAMPLE 52.

¹H NMR (CDCl₃): δ 1.46 (s, 9H), 1.80-1.91 (m, 4H), 2.08-2.27 (m, 4H), 2.62 (s, 3H), 3.71 (ddd, J=9.2, 4.3, 1.8 Hz, 1H), 4.01 (s, 2H), 4.30 (brs, 1H), 4.48 (dd, J=14.1, 9.2 Hz, 1H), 4.53 (brs, 1H), 5.01 (dd, J=14.1, 1.8 Hz, 1H), 6.71 (d, J=9.2 Hz, 1H), 7.05 (d, J=7.9 Hz, 1H), 7.61 (d, J=9.2 Hz, 1H), 7.76 (d, J=7.9 Hz, 1H).

MS (ESI⁺) m/z: 430 (MH⁺).

HRMS (ESI⁺) for C₂₃H₃₂N₃O₅ (MH⁺): calcd, 430.23420. found, 430.23387.

Enantiomer B of tert-butyl (1-(1-hydroxy-2-(7-methyl-2-oxo-1,8-naphthyridin-1(2H)-yl)ethyl)-2-oxabicyclo[2.2.2]octan-4-yl)carbamate (72.0 mg) was prepared in the same manner from 7-methyl-1,8-naphthyridin-2(1H)-one (60.0 mg) and AB (165 mg, Enantiomer B).

¹H NMR (CDCl₃): δ 1.44 (s, 9H), 1.80-1.91 (m, 4H), 2.09-2.27 (m, 4H), 2.62 (s, 3H), 3.71 (ddd, J=9.2, 4.3, 1.8 Hz, 1H), 4.01 (s, 2H), 4.30 (br, 1H), 4.48 (dd, J=14.1, 9.2 Hz, 1H), 4.53 (br, 1H), 5.01 (dd, J=14.1, 1.8 Hz, 1H), 6.71 (d, J=9.2 Hz, 1H), 7.05 (d, J=7.9 Hz, 1H), 7.61 (d, J=9.2 Hz, 1H), 7.76 (d, J=7.9 Hz, 1H).

MS (ESI⁺) m/z: 430 (MH⁺).

HRMS (ESI⁺) for C₂₃H₃₂N₃O₅ (MH⁺): calcd, 430.23420. found, 430.23444.

### Step 2

### 1-(2-(4-Amino-2-oxabicyclo[2.2.2]octan-1-yl)-2-hydroxyethyl)-7-methyl-1,8-naphthyridin-2(1H)-one (Enantiomer A)

The title compound 1-(2-(4-amino-2-oxabicyclo[2.2.2]octan-1-yl)-2-hydroxyethyl)-7-methyl-1,8-naphthyridin-2(1H)-one (45.5 mg) was prepared from tert-butyl (1-(1-hydroxy-2-(7-methyl-2-oxo-1,8-naphthyridin-1(2H)-yl)ethyl)-2-oxabicyclo[2.2.2]octan-4-yl)carbamate (56.0 mg, Enantiomer A) in the same manner as described for Step 2 of EXAMPLE 32.

¹H NMR (DMSO-d₆): δ 1.63-2.08 (m, 8H), 2.55 (s, 3H), 3.46-3.53 (m, 2H), 3.76 (ddd, J=9.1, 6.1, 3.6 Hz, 1H), 4.35 (dd, J=12.7, 3.6 Hz, 1H), 4.46 (d, J=6.1 Hz, 1H), 4.66 (dd, J=12.7, 9.1 Hz, 1H), 6.58 (d, J=9.7 Hz, 1H), 7.15 (d, J=7.9 Hz, 1H), 7.86 (d, J=9.7 Hz, 1H), 8.01 (d, J=7.9 Hz, 1H).

MS (ESI⁺) m/z: 330 (MH⁺).

HRMS (ESI⁺) for C₁₈H₂₄N₃O₃ (MH⁺): calcd, 330.18177. found, 330.18170.

Enantiomer B of 1-(2-(4-amino-2-oxabicyclo[2.2.2]octan-1-yl)-2-hydroxyethyl)-7-methyl-1,8-naphthyridin-2(1H)-one (48.0 mg) was prepared in the same manner from tert-butyl (1-(1-hydroxy-2-(7-methyl-2-oxo-1,8-naphthyridin-1(2H)-yl)ethyl)-2-oxabicyclo[2.2.2]octan-4-yl)carbamate (60.0 mg, Enantiomer B).

¹H NMR (DMSO-d₆): δ 1.44-2.12 (m, 8H), 2.54 (s, 3H), 3.36-3.46 (m, 2H), 3.74 (ddd, J=9.2, 6.1, 3.7 Hz, 1H), 4.34 (d, J=6.1 Hz, 1H), 4.35 (dd, J=14.7, 3.7 Hz, 1H), 4.65 (dd, J=12.8, 9.2 Hz, 1H), 6.58 (d, J=9.8 Hz, 1H), 7.15 (d, J=7.3 Hz, 1H), 7.85 (d, J=9.8 Hz, 1H), 8.01 (d, J=7.3 Hz, 1H).

MS (ESI⁺) m/z: 330 (MH⁺).

HRMS (ESI⁺) for C₁₈H₂₄N₃O₃ (MH⁺): calcd, 330.18177. found, 330.18159.

### Step 3

### 6-(((1-(1-Hydroxy-2-(7-methyl-2-oxo-1,8-naphthyridin-1(2H)-yl)ethyl)-2-oxabicyclo[2.2.2]octan-4-yl)amino)methyl)-2H-pyrido[3,2-b][1,4]oxazin-3(4H)-one (Enantiomer A)

The title compound 6-(((1-(1-hydroxy-2-(7-methyl-2-oxo-1,8-naphthyridin-1(2H)-yl)ethyl)-2-oxabicyclo[2.2.2]octan-4-yl)amino)methyl)-2H-pyrido[3,2-b][1,4]oxazin-3(4H)-one (23.0 mg) was prepared from 1-(2-(4-amino-2-oxabicyclo[2.2.2]octan-1-yl)-2-hydroxyethyl)-7-methyl-1,8-naphthyridin-2(1H)-one (45.0 mg, Enantiomer A) and I (25.6 mg) in the same manner as described for Step 3 of EXAMPLE 1.

¹H NMR (CDCl₃): δ 1.85-1.89 (m, 6H), 2.17-2.28 (m, 2H), 2.63 (s, 3H), 3.71-3.75 (m, 1H), 3.79 (s, 2H), 3.84 (s, 2H), 4.49 (dd, J=13.4, 8.6 Hz, 1H), 4.60 (d, J=4.3 Hz, 1H), 4.63 (s, 2H), 5.02 (dd, J=13.4, 1.8 Hz, 1H), 6.71 (d, J=9.2 Hz, 1H), 6.96 (d, J=7.9 Hz, 1H), 7.05 (d, J=7.3 Hz, 1H), 7.20 (d, J=7.9 Hz, 1H), 7.62 (d, J=9.8 Hz, 1H), 7.77 (d, J=7.3 Hz, 1H).

MS (ESI⁺) m/z: 492 (MH⁺).

HRMS (ESI⁺) for C₂₆H₃₀N₅O₅ (MH⁺): calcd, 492.22496. found, 492.22500.

Enantiomer B of 6-(((1-(1-hydroxy-2-(7-methyl-2-oxo-1,8-naphthyridin-1(2H)-yl)ethyl)-2-oxabicyclo[2.2.2]octan-4-yl)amino)methyl)-2H-pyrido[3,2-b][1,4]oxazin-3(4H)-one (36.0 mg) was prepared in the same manner from 1-(2-(4-amino-2-oxabicyclo[2.2.2]octan-1-yl)-2-hydroxyethyl)-7-methyl-1,8-naphthyridin-2(1H)-one (44.0 mg, Enantiomer B) and I (23.8 mg).

¹H NMR (CDCl₃): δ 1.85-1.89 (m, 6H), 2.17-2.28 (m, 2H), 2.63 (s, 3H), 3.73-3.75 (m, 1H), 3.79 (s, 2H), 3.84 (s, 2H), 4.49 (dd, J=13.4, 8.6 Hz, 1H), 4.60 (d, J=4.3 Hz, 1H), 4.63 (s, 2H), 5.02 (dd, J=13.4, 1.8 Hz, 1H), 6.71 (d, J=9.8 Hz, 1H), 6.96 (d, J=7.9 Hz, 1H), 7.05 (d, J=7.9 Hz, 1H), 7.20 (d, J=7.9 Hz, 1H), 7.62 (d, J=9.2 Hz, 1H), 7.77 (d, J=7.9 Hz, 1H).

MS (ESI⁺) m/z: 492 (MH⁺).

HRMS (ESI⁺) for C₂₆H₃₀N₅O₅ (MH⁺): calcd, 492.22496. found, 492.22437.

### Example 54

### 6-(((1-(1-Hydroxy-2-(7-methoxy-2-oxo-1,5-naphthyridin-1(2H)-yl)ethyl)-2-oxabicyclo[2.2.2]octan-4-yl)amino)methyl)-2H-pyrido[3,2-b][1,4]oxazin-3(4H)-one (Enantiomer A and Enantiomer B)

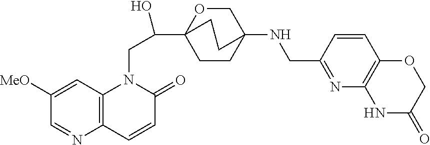 [SMILES: COc1cnc2ccc(=O)n(CC(O)C34CCC(NCc5ccc6c(n5)NC(=O)CO6)(CC3)CO4)c2c1]

### Step 1

### tert-Butyl (1-(1-Hydroxy-2-(7-methoxy-2-oxo-1,5-naphthyridin-1(2H)-yl)ethyl)-2-oxabicyclo[2.2.2]octan-4-yl)carbamate (Enantiomer A)

The title compound tert-butyl (1-(1-hydroxy-2-(7-methoxy-2-oxo-1,5-naphthyridin-1(2H)-yl)ethyl)-2-oxabicyclo[2.2.2]octan-4-yl)carbamate (92.0 mg) was prepared from 7-methoxy-1,5-naphthyridin-2(1H)-one (70.0 mg) and AB (175 mg, Enantiomer A) in the same manner as described for EXAMPLE 52.

¹H NMR (CDCl₃): δ 1.44 (s, 9H), 1.78-2.27 (m, 8H), 3.69 (d, J=7.9 Hz, 1H), 3.94 (s, 3H), 4.06 (d, J=4.9 Hz, 1H), 4.14 (s, 2H), 4.36-4.49 (m, 3H), 6.78 (d, J=9.8 Hz, 1H), 7.52 (d, J=2.4 Hz, 1H), 7.91 (d, J=9.8 Hz, 1H), 8.30 (d, J=2.4 Hz, 1H).

MS (ESI⁺) m/z: 446 (MH⁺).

HRMS (ESI⁺) for C₂₃H₃₂N₃O₆ (MH⁺): calcd, 446.22911. found, 446.22879.

Enantiomer B of tert-butyl (1-(1-hydroxy-2-(7-methoxy-2-oxo-1,5-naphthyridin-1(2H)-yl)ethyl)-2-oxabicyclo[2.2.2]octan-4-yl)carbamate (100 mg) was prepared in the same manner from 7-methoxy-1,5-naphthyridin-2(1H)-one (70.0 mg) and AB (175 mg, Enantiomer B).

¹H NMR (CDCl₃): δ 1.44 (s, 9H), 1.78-2.22 (m, 8H), 3.69 (d, J=7.9 Hz, 1H), 3.95 (s, 3H), 4.06 (d, J=5.5 Hz, 1H), 4.14 (s, 2H), 4.36-4.49 (m, 3H), 6.78 (d, J=9.8 Hz, 1H), 7.52 (d, J=1.8 Hz, 1H), 7.92 (d, J=9.8 Hz, 1H), 8.30 (d, J=2.4 Hz, 1H).

MS (ESI⁺) m/z: 446 (MH⁺).

HRMS (ESI⁺) for C₂₃H₃₂N₃O₆ (MH⁺): calcd, 446.22911. found, 446.22998.

### Step 2

### 1-(2-(4-Amino-2-oxabicyclo[2.2.2]octan-1-yl)-2-hydroxyethyl)-7-methoxy-1,5-naphthyridin-2(1H)-one (Enantiomer A)

The title compound 1-(2-(4-amino-2-oxabicyclo[2.2.2]octan-1-yl)-2-hydroxyethyl)-7-methoxy-1,5-naphthyridin-2(1H)-one (52.2 mg) was prepared from tert-butyl (1-(1-hydroxy-2-(7-methoxy-2-oxo-1,5-naphthyridin-1(2H)-yl)ethyl)-2-oxabicyclo[2.2.2]octan-4-yl)carbamate (75.0 mg, Enantiomer A) in the same manner as described for Step 2 of EXAMPLE 32.

¹H NMR (DMSO-d₆): δ 1.66-2.15 (m, 8H), 3.61-3.75 (m, 3H), 3.95 (s, 3H), 4.40-4.48 (m, 2H), 6.78 (d, J=9.8 Hz, 1H), 7.55 (d, J=2.4 Hz, 1H), 7.91 (d, J=9.8 Hz, 1H), 8.30 (d, J=2.4 Hz, 1H).

MS (ESI⁺) m/z: 346 (MH⁺).

HRMS (ESI⁺) for C₁₈H₂₄N₃O₄ (MH⁺): calcd, 346.17668. found, 346.17722.

Enantiomer B of 1-(2-(4-amino-2-oxabicyclo[2.2.2]octan-1-yl)-2-hydroxyethyl)-7-methoxy-1,5-naphthyridin-2(1H)-one (60.3 mg) was prepared in the same manner from tert-butyl (1-(1-hydroxy-2-(7-methoxy-2-oxo-1,5-naphthyridin-1(2H)-yl)ethyl)-2-oxabicyclo[2.2.2]octan-4-yl)carbamate (84.0 mg, Enantiomer B).

MS (ESI⁺) m/z: 346 (MH⁺).

HRMS (ESI⁺) for C₁₈H₂₄N₃O₄ (MH⁺): calcd, 346.17668. found, 346.17589.

### Step 3

### 6-(((1-(1-Hydroxy-2-(7-methoxy-2-oxo-1,5-naphthyridin-1(2H)-yl)ethyl)-2-oxabicyclo[2.2.2]octan-4-yl)amino)methyl)-2H-pyrido[3,2-b][1,4]oxazin-3(4H)-one (Enantiomer A)

The title compound 6-(((1-(1-hydroxy-2-(7-methoxy-2-oxo-1,5-naphthyridin-1(2H)-yl)ethyl)-2-oxabicyclo[2.2.2]octan-4-yl)amino)methyl)-2H-pyrido[3,2-b][1,4]oxazin-3(4H)-one (30.0 mg) was prepared from 1-(2-(4-amino-2-oxabicyclo[2.2.2]octan-1-yl)-2-hydroxyethyl)-7-methoxy-1,5-naphthyridin-2(1H)-one (47.0 mg, Enantiomer A) and I (26.7 mg) in the same manner as described for Step 3 of EXAMPLE 1.

¹H NMR (CDCl₃): δ 1.78-2.15 (m, 8H), 3.70-3.75 (m, 2H), 3.77 (s, 2H), 3.82 (dd, J=7.9, 2.4 Hz, 1H), 3.86 (dd, J=7.9, 2.4 Hz, 1H), 3.95 (s, 3H), 4.07 (s, 1H), 4.44 (s, 1H), 4.45 (d, J=1.8 Hz, 1H), 4.64 (s, 2H), 6.78 (d, J=9.8 Hz, 1H), 6.95 (d, J=7.9 Hz, 1H), 7.21 (d, J=7.9 Hz, 1H), 7.55 (d, J=2.4 Hz, 1H), 7.92 (d, J=9.8 Hz, 1H), 8.08 (brs, 1H), 8.31 (d, J=2.4 Hz, 1H).

MS (ESI⁺) m/z: 508 (MH⁺).

HRMS (ESI⁺) for C₂₆H₃₀N₅O₆ (MH⁺): calcd, 508.21961. found, 508.21932.

Enantiomer B of 6-(((1-(1-hydroxy-2-(7-methoxy-2-oxo-1,5-naphthyridin-1(2H)-yl)ethyl)-2-oxabicyclo[2.2.2]octan-4-yl)amino)methyl)-2H-pyrido[3,2-b][1,4]oxazin-3(4H)-one (38.0 mg) was prepared in the same manner from 1-(2-(4-amino-2-oxabicyclo[2.2.2]octan-1-yl)-2-hydroxyethyl)-7-methoxy-1,5-naphthyridin-2(1H)-one (51.6 mg, Enantiomer B) and I (29.3 mg).

¹H NMR (CDCl₃): δ 1.77-2.13 (m, 8H), 3.70-3.75 (m, 2H), 3.77 (s, 2H), 3.82 (dd, J=7.9, 2.4 Hz, 1H), 3.86 (dd, J=7.9, 2.4 Hz, 1H), 3.95 (s, 3H), 4.06 (s, 1H), 4.43 (s, 1H), 4.45 (d, J=1.8 Hz, 1H), 4.64 (s, 2H), 6.78 (d, J=9.8 Hz, 1H), 6.95 (d, J=8.6 Hz, 1H), 7.21 (d, J=7.9 Hz, 1H), 7.55 (d, J=2.4 Hz, 1H), 7.92 (d, J=9.8 Hz, 1H), 8.25 (brs, 1H), 8.30 (d, J=2.4 Hz, 1H).

MS (ESI⁺) m/z: 508 (MH⁺).

HRMS (ESI⁺) for C₂₆H₃₀N₅O₆ (MH⁺): calcd, 508.21961. found, 508.21902.

### Example 55

### 6-((1-(2-(7-Fluoro-2-oxo-1,5-naphthyridin-1(2H)-yl)-1-hydroxyethyl)-2-oxabicyclo[2.2.2]octan-4-ylamino)methyl)-2H-pyrido[3,2-b][1,4]oxazin-3(4H)-one

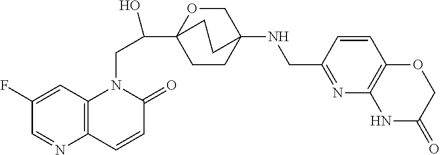 [SMILES: O=C1COc2ccc(CNC34CCC(C(O)Cn5c(=O)ccc6ncc(F)cc65)(CC3)OC4)nc2N1]

### Step 1

### tert-Butyl 1-(2-(7-Fluoro-2-oxo-1,5-naphthyridin-1(2H)-yl)-1-hydroxyethyl)-2-oxabicyclo[2.2.2]octan-4-ylcarbamate (Enantiomer A and Enantiomer B)

The title compound tert-butyl 1-(2-(7-fluoro-2-oxo-1,5-naphthyridin-1(2H)-yl)-1-hydroxyethyl)-2-oxabicyclo[2.2.2]octan-4-ylcarbamate (120 mg) was prepared from 7-fluoro-1,5-naphthyridin-2(1H)-one (390 mg) and AA (704 mg) in the same manner as described for Step 1 of EXAMPLE 52. Optical resolution (CHIRALPAK IA, hexane:ethanol=30:70) of the racemate (100 mg) gave Enantiomer A and Enantiomer B.

### Step 2

### 1-(2-(4-Amino-2-oxabicyclo[2.2.2]octan-1-yl)-2-hydroxyethyl)-7-fluoro-1,5-naphthyridin-2(1H)-one (Enantiomer A)

The title compound 1-(2-(4-amino-2-oxabicyclo[2.2.2]octan-1-yl)-2-hydroxyethyl)-7-fluoro-1,5-naphthyridin-2(1H)-one (28.4 mg) was prepared from tert-butyl 1-(2-(7-fluoro-2-oxo-1,5-naphthyridin-1(2H)-yl)-1-hydroxyethyl)-2-oxabicyclo[2.2.2]octan-4-ylcarbamate (41.0 mg, Enantiomer A) in the same manner as described for Step 2 of EXAMPLE 32.

Enantiomer B of 1-(2-(4-amino-2-oxabicyclo[2.2.2]octan-1-yl)-2-hydroxyethyl)-7-fluoro-1,5-naphthyridin-2(1H)-one (29.0 mg) was prepared in the same manner from tert-butyl 1-(2-(7-fluoro-2-oxo-1,5-naphthyridin-1(2H)-yl)-1-hydroxyethyl)-2-oxabicyclo[2.2.2]octan-4-ylcarbamate (41.0 mg, Enantiomer B).

### Step 3

### 6-((1-(2-(7-Fluoro-2-oxo-1,5-naphthyridin-1(2H)-yl)-1-hydroxyethyl)-2-oxabicyclo[2.2.2]octan-4-ylamino)methyl)-2H-pyrido[3,2-b][1,4]oxazin-3(4H)-one (Enantiomer A)

The title compound 6-((1-(2-(7-fluoro-2-oxo-1,5-naphthyridin-1(2H)-yl)-1-hydroxyethyl)-2-oxabicyclo[2.2.2]octan-4-ylamino)methyl)-2H-pyrido[3,2-b][1,4]oxazin-3(4H)-one (25.2 mg) was prepared from 1-(2-(4-amino-2-oxabicyclo[2.2.2]octan-1-yl)-2-hydroxyethyl)-7-fluoro-1,5-naphthyridin-2(1H)-one (26.0 mg, Enantiomer A) and I (14.9 mg) in the same manner as described for Step 3 of EXAMPLE 1.

¹H NMR (DMSO-d₆): δ 1.63-1.99 (m, 9H), 3.55-3.63 (m, 5H), 4.12 (dd, J=14.7, 10.4 Hz, 1H), 4.37 (dd, J=14.1, 2.4 Hz, 1H), 4.59 (s, 2H), 4.94 (d, J=6.1 Hz, 1H), 6.81 (d, J=9.8 Hz, 1H), 7.02 (d, J=7.9 Hz, 1H), 7.28 (d, J=8.6 Hz, 1H), 7.86 (dd, J=11.0, 1.8 Hz, 1H), 7.93 (d, J=9.8 Hz, 1H), 8.52 (d, J=1.8 Hz, 1H), 11.15 (s, 1H).

MS (ESI⁺) m/z: 496 (MH⁺).

HRMS (ESI⁺) for C₂₅H₂₇FN₅O₅ (MH⁺): calcd, 496.19962. found, 496.19909.

Enantiomer B of 6-((1-(2-(7-fluoro-2-oxo-1,5-naphthyridin-1(2H)-yl)-1-hydroxyethyl)-2-oxabicyclo[2.2.2]octan-4-ylamino)methyl)-2H-pyrido[3,2-b][1,4]oxazin-3(4H)-one (28.0 mg) was prepared in the same manner from 1-(2-(4-amino-2-oxabicyclo[2.2.2]octan-1-yl)-2-hydroxyethyl)-7-fluoro-1,5-naphthyridin-2(1H)-one (25.5 mg, Enantiomer B) and I (14.3 mg).

¹H NMR (DMSO-d₆): δ 1.63-2.05 (m, 9H), 3.55-3.63 (m, 5H), 4.11 (dd, J=14.1, 9.8 Hz, 1H), 4.36 (d, J=15.9 Hz, 1H), 4.59 (s, 2H), 4.93 (d, J=6.1 Hz, 1H), 6.81 (d, J=9.8 Hz, 1H), 7.01 (d, J=7.9 Hz, 1H), 7.28 (d, J=7.9 Hz, 1H), 7.87 (dd, J=11.0, 1.8 Hz, 1H), 7.93 (d, J=9.8 Hz, 1H), 8.51 (d, J=1.8 Hz, 1H), 11.15 (s, 1H).

MS (ESI⁺) m/z: 496 (MH⁺).

HRMS (ESI⁺) for C₂₅H₂₇FN₅O₅ (MH⁺): calcd, 496.19962. found, 496.19910.

The following examples EXAMPLE 56-EXAMPLE 58 were prepared from 4-(2-(3-chloro-6-methoxy-1,5-naphthyridin-4-yl)ethyl)bicyclo[2.2.2]octan-1-amine and corresponding aldehydes in the same manner as described for Step 3 of EXAMPLE 1.

### Example 56

### 6-((4-(2-(3-Chloro-6-methoxy-1,5-naphthyridin-4-yl)ethyl)bicyclo[2.2.2]octan-1-ylamino)methyl)-2H-benzo[b][1,4]oxazin-3(4H)-one

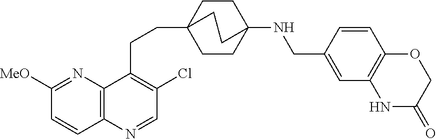 [SMILES: COc1ccc2ncc(Cl)c(CCC34CCC(NCc5ccc6c(c5)NC(=O)CO6)(CC3)CC4)c2n1]

The title compound was prepared starting with 3-oxo-3,4-dihydro-2H-benzo[b][1,4]oxazine-6-carbaldehyde.

¹H NMR (DMSO-d₆): δ 1.33-1.42 (m, 2H), 1.58 (s, 12H), 3.08-3.21 (m, 2H), 3.57 (brs, 2H), 4.03 (s, 3H), 4.52 (s, 2H), 6.79-6.96 (m, 3H), 7.27 (d, J=8.6 Hz, 1H), 8.26 (d, J=8.6 Hz, 1H), 8.72 (s, 1H), 10.64 (brs, 1H).

MS (ESI⁺) m/z: 507 (MH⁺).

HRMS (ESI⁺) for C₂₈H₃₂ClN₄O₃ (MH⁺): calcd, 507.21629. found, 507.21586.

### Example 57

### 4-(2-(3-Chloro-6-methoxy-1,5-naphthyridin-4-yl)ethyl)-N-((6-methoxyquinolin-2-yl)methyl)bicyclo[2.2.2]octan-1-amine

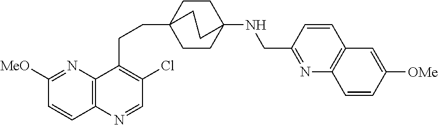 [SMILES: COc1ccc2nc(CNC34CCC(CCc5c(Cl)cnc6ccc(OC)nc56)(CC3)CC4)ccc2c1]

The title compound was prepared starting with 6-methoxyquinoline-2-carbaldehyde.

¹H NMR (DMSO-d₆): δ 1.29-1.40 (m, 2H), 1.58 (s, 12H), 3.12-3.23 (m, 2H), 3.87 (s, 3H), 3.91 (s, 2H), 4.02 (s, 3H), 7.27 (d, J=9.2 Hz, 1H), 7.32-7.38 (m, 2H), 7.55 (d, J=8.6 Hz, 1H), 7.84 (d, J=9.2 Hz, 1H), 8.17 (d, J=8.6 Hz, 1H), 8.25 (d, J=8.6 Hz, 1H), 8.72 (s, 1H).

MS (ESI⁺) m/z: 517 (MH⁺).

HRMS (ESI⁺) for C₃₀H₃₄ClN₄O₂ (MH⁺): calcd, 517.23703. found, 517.23724.

### Example 58

### N-((1,8-Naphthyridin-2-yl)methyl)-4-(2-(3-chloro-6-methoxy-1,5-naphthyridin-4-yl)ethyl)bicyclo[2.2.2]octan-1-amine

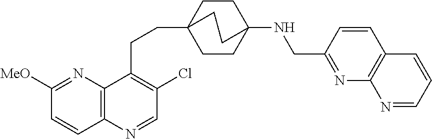 [SMILES: COc1ccc2ncc(Cl)c(CCC34CCC(NCc5ccc6cccnc6n5)(CC3)CC4)c2n1]

The title compound was prepared starting from 1,8-naphthyridine-2-carbaldehyde.

¹H NMR (DMSO-d₆): δ 1.30-1.39 (m, 2H), 1.59 (s, 12H), 2.17 (brs, 1H), 3.12-3.21 (m, 2H), 3.99 (s, 2H), 4.08 (s, 3H), 7.27 (d, J=9.2 Hz, 1H), 7.57 (dd, J=7.9, 4.3 Hz, 1H), 7.73 (d, J=8.6 Hz, 1H), 8.26 (d, J=8.6 Hz, 1H), 8.37 (d, J=8.6 Hz, 1H), 8.42 (dd, J=7.9, 2.4 Hz, 1H), 8.72 (s, 1H), 9.02 (dd, J=4.3, 2.0 Hz, 1H).

MS (ESI⁺) m/z: 488 (MH⁺).

HRMS (ESI⁺) for C₂₈H₃₁ClN₅O (MH⁺): calcd, 488.22171. found, 488.22159.

The following examples EXAMPLES 59-108 were prepared from 4-(2-(3-fluoro-6-methoxy-1,5-naphthyridin-4-yl)ethyl)-2-oxabicyclo[2.2.2]octan-1-amine and corresponding aldehydes, acyl chlorides, or sulfonyl chlorides in the same manner as described for Step 3 of EXAMPLE 1.

### Example 59

### N-(3-Fluoro-4-methylbenzyl)-1-(2-(3-fluoro-6-methoxy-1,5-naphthyridin-4-yl)ethyl)-2-oxabicyclo[2.2.2]octan-4-amine

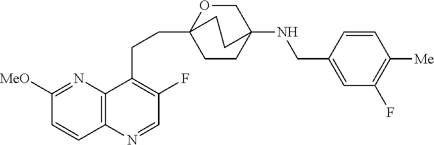 [SMILES: COc1ccc2ncc(F)c(CCC34CCC(NCc5ccc(C)c(F)c5)(CC3)CO4)c2n1]

The title compound was prepared from 3-fluoro-4-methylbenzaldehyde.

¹H NMR (DMSO-d₆): δ 1.56-1.79 (m, 8H), 1.84-1.96 (m, 3H), 2.18 (s, 3H), 3.06-3.15 (m, 2H), 3.57 (s, 2H), 3.61 (s, 2H), 4.02 (s, 3H), 7.03 (d, J=7.3 Hz, 1H), 7.16 (d, J=11.0 Hz, 1H), 7.16 (t, J=7.9 Hz, 1H), 7.22 (d, J=9.2 Hz, 1H), 8.26 (d, J=9.2 Hz, 1H), 8.74 (s, 1H).

MS (ESI⁺) m/z: 454 (MH⁺).

HRMS (ESI⁺) for C₂₆H₃₀F₂N₃O₂ (MH⁺): calcd, 454.23061. found, 454.23064.

### Example 60

### 6-(((1-(2-(3-Fluoro-6-methoxy-1,5-naphthyridin-4-yl)ethyl)-2-oxabicyclo[2.2.2]octan-4-yl)amino)methyl)picolinonitrile

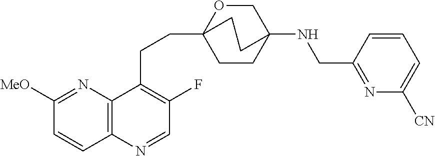 [SMILES: COc1ccc2ncc(F)c(CCC34CCC(NCc5cccc(C#N)n5)(CC3)CO4)c2n1]

The title compound was prepared from 6-formylpyridine-2-carbonitrile.

¹H NMR (DMSO-d₆): δ 1.57-1.77 (m, 8H), 1.81-1.93 (m, 2H), 2.24 (br, 1H), 3.05-3.16 (m, 2H), 3.58 (s, 2H), 3.81 (s, 2H), 4.03 (s, 3H), 7.22 (d, J=9.2 Hz, 1H), 7.81 (d, J=7.9 Hz, 1H), 7.87 (d, J=7.9 Hz, 1H), 7.99 (t, J=7.9 Hz, 1H), 8.26 (d, J=9.2 Hz, 1H), 8.74 (s, 1H).

MS (ESI⁺) m/z: 448 (MH⁺).

HRMS (ESI⁺) for C₂₅H₂₇FN₅O₂ (MH⁺): calcd, 448.21488. found, 448.21439.

### Example 61

### N-((2,3-Dihydro-[1,4]dioxino[2,3-c]pyridin-7-yl)methyl)-1-(2-(3-fluoro-6-methoxy-1,5-naphthyridin-4-yl)ethyl)-2-oxabicyclo[2.2.2]octan-4-amine

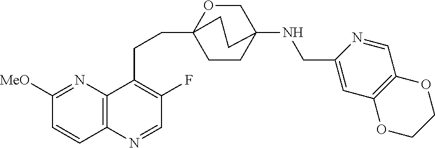 [SMILES: COc1ccc2ncc(F)c(CCC34CCC(NCc5cc6c(cn5)OCCO6)(CC3)CO4)c2n1]

The title compound was prepared from 2,3-dihydro[1,4]dioxino[2,3-c]pyridine-7-carbaldehyde.

¹H NMR (DMSO-d₆): δ 1.55-2.21 (m, 11H), 3.05-3.17 (m, 2H), 3.56 (s, 2H), 3.61 (d, J=5.5 Hz, 2H), 4.03 (s, 3H), 4.25-4.28 (m, 2H), 4.29-4.37 (m, 2H), 6.92 (s, 1H), 7.22 (d, J=9.2 Hz, 1H), 7.98 (s, 1H), 8.26 (d, J=9.2 Hz, 1H), 8.74 (s, 1H).

MS (ESI⁺) m/z: 481 (MH⁺).

HRMS (ESI⁺) for C₂₆H₃₀FN₄O₄ (MH⁺): calcd, 481.22511. found, 481.22542.

### Example 62

### N-((4,5-Dimethoxypyridin-2-yl)methyl)-1-(2-(3-fluoro-6-methoxy-1,5-naphthyridin-4-yl)ethyl)-2-oxabicyclo[2.2.2]octan-4-amine

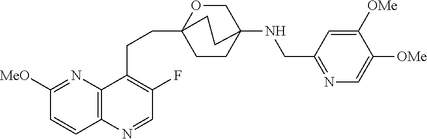 [SMILES: COc1ccc2ncc(F)c(CCC34CCC(NCc5cc(OC)c(OC)cn5)(CC3)CO4)c2n1]

The title compound was prepared from 4,5-dimethoxypyridine-2-carbaldehyde.

¹H NMR (DMSO-d₆): δ 1.58-1.72 (m, 8H), 1.77-2.12 (m, 3H), 3.07-3.15 (m, 2H), 3.58 (s, 2H), 3.66 (s, 2H), 3.79 (s, 3H), 3.81 (s, 3H), 4.03 (s, 3H), 7.04 (s, 1H), 7.22 (d, J=9.2 Hz, 1H), 8.01 (s, 1H), 8.26 (d, J=9.2 Hz, 1H), 8.74 (s, 1H).

MS (ESI⁺) m/z: 483 (MH⁺).

HRMS (ESI⁺) for C₂₆H₃₂FN₄O₄ (MH⁺): calcd, 483.24076. found, 483.24004.

### Example 63

### 1-(2-(3-Fluoro-6-methoxy-1,5-naphthyridin-4-yl)ethyl)-N-((5-(pyrrolidin-1-yl)pyridin-2-yl)methyl)-2-oxabicyclo[2.2.2]octan-4-amine

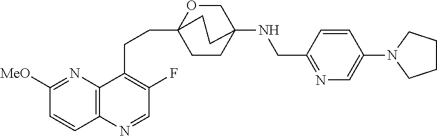 [SMILES: COc1ccc2ncc(F)c(CCC34CCC(NCc5ccc(N6CCCC6)cn5)(CC3)CO4)c2n1]

The title compound was prepared from 5-(pyrrolidin-1-yl)pyridine-2-carbaldehyde.

¹H NMR (DMSO-d₆): δ 1.57-1.91 (m, 11H), 1.91-1.99 (m, 4H), 3.07-3.15 (m, 2H), 3.17-3.24 (m, 4H), 3.56 (s, 2H), 3.62 (s, 2H), 4.03 (s, 3H), 6.85 (dd, J=8.6, 2.4 Hz, 1H), 7.16 (d, J=8.6 Hz, 1H), 7.22 (d, J=9.2 Hz, 1H), 7.80 (d, J=3.1 Hz, 1H), 8.26 (d, J=9.2 Hz, 1H), 8.74 (s, 1H).

MS (ESI⁺) m/z: 492 (MH⁺).

HRMS (ESI⁺) for C₂₈H₃₅FN₅O₂ (MH⁺): calcd, 492.27748. found, 492.27701.

### Example 64

### 1-(2-(3-Fluoro-6-methoxy-1,5-naphthyridin-4-yl)ethyl)-N-((6-morpholinopyridin-3-yl)methyl)-2-oxabicyclo[2.2.2]octan-4-amine

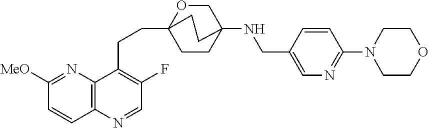 [SMILES: COc1ccc2ncc(F)c(CCC34CCC(NCc5ccc(N6CCOCC6)nc5)(CC3)CO4)c2n1]

The title compound was prepared from 6-(morpholin-4-yl)pyridine-3-carbaldehyde.

¹H NMR (DMSO-d₆): δ 1.59-1.78 (m, 9H), 1.80-1.93 (m, 2H), 3.06-3.16 (m, 2H), 3.36 (t, J=4.9 Hz, 4H), 3.51 (s, 2H), 3.57 (s, 2H), 3.68 (t, J=4.3 Hz, 4H), 4.03 (s, 3H), 6.76 (d, J=8.6 Hz, 1H), 7.22 (d, J=9.1 Hz, 1H), 7.50 (dd, J=8.6, 2.4 Hz, 1H), 8.03 (d, J=2.4 Hz, 1H), 8.26 (d, J=9.1 Hz, 1H), 8.74 (s, 1H).

MS (ESI⁺) m/z: 508 (MH⁺).

HRMS (ESI⁺) for C₂₈H₃₅FN₅O₃ (MH⁺): calcd, 508.27239. found, 508.27268.

### Example 65

### 1-(2-(3-Fluoro-6-methoxy-1,5-naphthyridin-4-yl)ethyl)-N-((2-morpholinopyrimidin-5-yl)methyl)-2-oxabicyclo[2.2.2]octan-4-amine

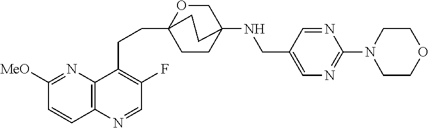 [SMILES: COc1ccc2ncc(F)c(CCC34CCC(NCc5cnc(N6CCOCC6)nc5)(CC3)CO4)c2n1]

The title compound was prepared from 2-(morpholin-4-yl)pyrimidine-5-carbaldehyde.

¹H NMR (DMSO-d₆): δ 1.58-1.77 (m, 8H), 1.80-1.92 (m, 3H), 3.07-3.15 (m, 2H), 3.48 (s, 2H), 3.57 (s, 2H), 3.63 (s, 8H), 4.03 (s, 3H), 7.22 (d, J=9.2 Hz, 1H), 8.26 (d, J=8.6 Hz, 1H), 8.29 (s, 2H), 8.74 (s, 1H).

MS (ESI⁺) m/z: 509 (MH⁺).

HRMS (ESI⁺) for C₂₇H₃₄FN₆O₃ (MH⁺): calcd, 509.26764. found, 509.26706.

### Example 66

### 1-(2-(3-Fluoro-6-methoxy-1,5-naphthyridin-4-yl)ethyl)-N-((4-methyl-3,4-dihydro-2H-benzo[b][1,4]oxazin-7-yl)methyl)-2-oxabicyclo[2.2.2]octan-4-amine

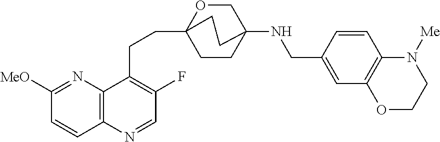 [SMILES: COc1ccc2ncc(F)c(CCC34CCC(NCc5ccc6c(c5)OCCN6C)(CC3)CO4)c2n1]

The title compound was prepared from 4-methyl-3,4-dihydro-2H-1,4-benzoxazine-7-carbaldehyde.

¹H NMR (DMSO-d₆): δ 1.48-1.90 (m, 11H), 2.77 (s, 3H), 3.07-3.18 (m, 4H), 3.43-3.49 (m, 2H), 3.56 (s, 2H), 4.03 (s, 3H), 4.16-4.21 (m, 2H), 6.58 (d, J=8.6 Hz, 1H), 6.61 (d, J=2.4 Hz, 1H), 6.68 (dd, J=7.9, 1.8 Hz, 1H), 7.22 (d, J=9.2 Hz, 1H), 8.26 (d, J=9.2 Hz, 1H), 8.74 (s, 1H).

MS (ESI⁺) m/z: 493 (MH⁺).

HRMS (ESI⁺) for C₂₈H₃₄FN₄O₃ (MH⁺): calcd, 493.26149. found, 493.26112.

### Example 67

### N-((8-Chloro-2,3-dihydrobenzo[b][1,4]dioxin-6-yl)methyl)-1-(2-(3-fluoro-6-methoxy-1,5-naphthyridin-4-yl)ethyl)-2-oxabicyclo[2.2.2]octan-4-amine

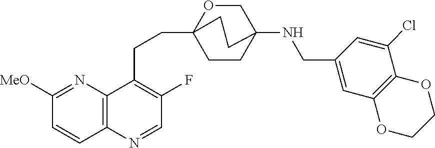 [SMILES: COc1ccc2ncc(F)c(CCC34CCC(NCc5cc(Cl)c6c(c5)OCCO6)(CC3)CO4)c2n1]

The title compound was prepared from 8-chloro-2,3-dihydro-1,4-benzodioxine-6-carbaldehyde.

¹H NMR (DMSO-d₆): δ 1.57-1.74 (m, 8H), 1.80-1.91 (m, 3H), 3.06-3.13 (m, 2H), 3.52 (s, 2H), 3.56 (s, 2H), 4.03 (s, 3H), 4.22-4.35 (m, 4H), 6.79 (d, J=2.4 Hz, 1H), 6.93 (d, J=1.8 Hz, 1H), 7.22 (d, J=9.2 Hz, 1H), 8.26 (d, J=9.2 Hz, 1H), 8.74 (s, 1H).

MS (ESI⁺) m/z: 514 (MH⁺).

HRMS (ESI⁺) for C₂₇H₃₀FN₃O₄ (MH⁺): calcd, 514.19089. found, 514.19056.

### Example 68

### 3-Fluoro-N-(1-(2-(3-fluoro-6-methoxy-1,5-naphthyridin-4-yl)ethyl)-2-oxabicyclo[2.2.2]octan-4-yl)-4-methylbenzamide

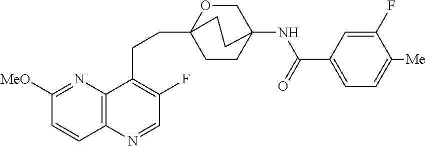 [SMILES: COc1ccc2ncc(F)c(CCC34CCC(NC(=O)c5ccc(C)c(F)c5)(CC3)CO4)c2n1]

The title compound was prepared from the corresponding acid chloride.

¹H NMR (DMSO-d₆): δ 1.59-1.72 (m, 2H), 1.73-1.85 (m, 2H), 1.87-2.08 (m, 4H), 2.09-2.20 (m, 2H), 2.26 (s, 3H), 3.06-3.19 (m, 2H), 3.98 (d, 2H), 4.04 (s, 3H), 7.23 (d, J=9.2 Hz, 1H), 7.34 (t, J=7.9 Hz, 1H), 7.52-7.60 (m, 2H), 7.79 (s, 1H), 8.27 (d, J=9.2 Hz, 1H), 8.75 (s, 1H).

MS (ESI⁺) m/z: 468 (MH⁺).

HRMS (ESI⁺) for C₂₆H₂₈F₂N₃O₃ (MH⁺): calcd, 468.20987. found, 468.20923.

### Example 69

### N-((2,3-Dihydrobenzo[b][1,4]dioxin-6-yl)methyl)-1-(2-(3-fluoro-6-methoxy-1,5-naphthyridin-4-yl)ethyl)-2-oxabicyclo[2.2.2]octan-4-amine

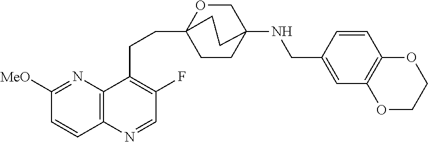 [SMILES: COc1ccc2ncc(F)c(CCC34CCC(NCc5ccc6c(c5)OCCO6)(CC3)CO4)c2n1]

The title compound was prepared from 2,3-dihydro-1,4-benzodioxine-6-carbaldehyde.

¹H NMR (DMSO-d₆): δ 1.56-1.76 (m, 9H), 1.79-1.90 (m, 2H), 3.05-3.14 (m, 2H), 3.51 (s, 2H), 3.57 (s, 2H), 4.03 (s, 3H), 4.18 (s, 4H), 6.73 (s, 2H), 6.79 (s, 1H), 7.22 (d, J=9.2 Hz, 1H), 8.26 (d, J=9.2 Hz, 1H), 8.74 (s, 1H).

MS (ESI⁺) m/z: 480 (MH⁺).

HRMS (ESI⁺) for C₂₇H₃₁FN₃O₄ (MH⁺): calcd, 480.22986. found, 480.22931.

### Example 70

### N-(Cyclohexylmethyl)-1-(2-(3-fluoro-6-methoxy-1,5-naphthyridin-4-yl)ethyl)-2-oxabicyclo[2.2.2]octan-4-amine

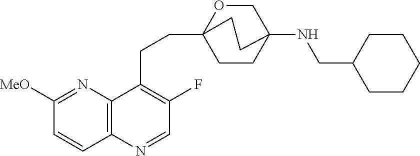 [SMILES: COc1ccc2ncc(F)c(CCC34CCC(NCC5CCCCC5)(CC3)CO4)c2n1]

The title compound was prepared from cyclohexanecarbaldehyde.

¹H NMR (DMSO-d₆): δ 0.76-0.89 (m, 2H), 1.12-1.26 (m, 5H), 1.50-1.75 (m, 13H), 1.76-1.89 (m, 2H), 2.21-2.50 (m, 2H), 3.05-3.14 (m, 2H), 3.51 (s, 2H), 4.02 (s, 3H), 7.22 (d, J=8.6 Hz, 1H), 8.25 (d, J=8.6 Hz, 1H), 8.74 (s, 1H).

MS (ESI⁺) m/z: 428 (MH⁺).

HRMS (ESI⁺) for C₂₅H₃₅FN₃O₂ (MH⁺): calcd, 428.27133. found, 428.27198.

### Example 71

### 3-Fluoro-N-(1-(2-(3-fluoro-6-methoxy-1,5-naphthyridin-4-yl)ethyl)-2-oxabicyclo[2.2.2]octan-4-yl)-4-methylbenzenesulfonamide

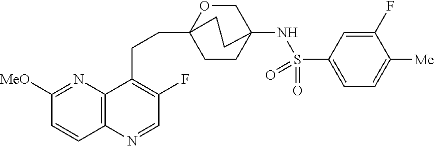 [SMILES: COc1ccc2ncc(F)c(CCC34CCC(NS(=O)(=O)c5ccc(C)c(F)c5)(CC3)CO4)c2n1]

The title compound was prepared from the corresponding sulfonyl chloride.

¹H NMR (DMSO-d₆): δ 1.51-1.58 (m, 2H), 1.61-1.69 (m, 4H), 1.73-1.86 (m, 4H), 2.28-2.33 (m, 3H), 3.00-3.07 (m, 2H), 3.61 (s, 2H), 3.98 (s, 3H), 7.20 (d, J=9.2 Hz, 1H), 7.48-7.59 (m, 3H), 7.83 (s, 1H), 8.24 (d, J=9.2 Hz, 1H), 8.72 (s, 1H).

MS (ESI⁺) m/z: 504 (MH⁺).

HRMS (ESI⁺) for C₂₅H₂₈F₂N₃O₄S (MH⁺): calcd, 504.17686. found, 504.17721.

### Example 72

### N-((7-Chlorobenzo[d][1,3]dioxol-5-yl)methyl)-1-(2-(3-fluoro-6-methoxy-1,5-naphthyridin-4-yl)ethyl)-2-oxabicyclo[2.2.2]octan-4-amine

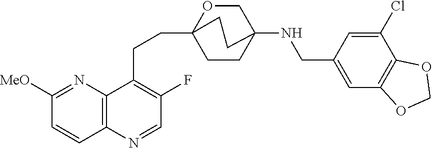 [SMILES: COc1ccc2ncc(F)c(CCC34CCC(NCc5cc(Cl)c6c(c5)OCO6)(CC3)CO4)c2n1]

The title compound was prepared from 7-chloro-1,3-benzodioxole-5-carbaldehyde.

¹H NMR (DMSO-d₆): δ 1.56-1.78 (m, 8H), 1.79-1.93 (m, 3H), 3.06-3.16 (m, 2H), 3.58 (s, 2H), 3.60 (s, 2H), 4.03 (s, 3H), 6.02 (s, 2H), 6.99 (s, 1H), 7.09 (s, 1H), 7.22 (d, J=9.2 Hz, 1H), 8.26 (d, J=9.2 Hz, 1H), 8.74 (s, 1H).

MS (ESI⁺) m/z: 500 (MH⁺).

HRMS (ESI⁺) for C₂₆H₂₈ClFN₃O₄ (MH⁺): calcd, 500.17524. found, 500.17606.

### Example 73

### 1-(2-(3-Fluoro-6-methoxy-1,5-naphthyridin-4-yl)ethyl)-N-((5-(thiophen-2-yl)isoxazol-3-yl)methyl)-2-oxabicyclo[2.2.2]octan-4-amine

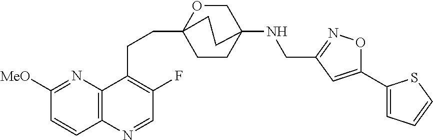 [SMILES: COc1ccc2ncc(F)c(CCC34CCC(NCc5cc(-c6cccs6)on5)(CC3)CO4)c2n1]

The title compound was prepared from 5-(thiophen-2-yl)isoxazole-3-carbaldehyde.

¹H NMR (DMSO-d₆): δ 1.58-1.78 (m, 8H), 1.81-1.93 (m, 2H), 2.15 (s, 1H), 3.06-3.17 (m, 2H), 3.59 (s, 2H), 3.71 (d, J=6.1 Hz, 2H), 4.03 (s, 3H), 6.80 (s, 1H), 7.19-7.25 (m, 2H), 7.67 (dd, J=3.7, 1.2 Hz, 1H), 7.79 (dd, J=4.9, 1.2 Hz, 1H), 8.26 (d, J=9.2 Hz, 1H), 8.74 (s, 1H).

MS (ESI) m/z: 495 (MH⁺).

HRMS (ESI⁺) for C₂₆H₂₈FN₄O₃S (MH⁺): calcd, 495.18661. found, 495.18741.

### Example 74

### 1-(2-(3-Fluoro-6-methoxy-1,5-naphthyridin-4-yl)ethyl)-N-((1-(pyridin-2-yl)-1H-pyrazol-4-yl)methyl)-2-oxabicyclo[2.2.2]octan-4-amine

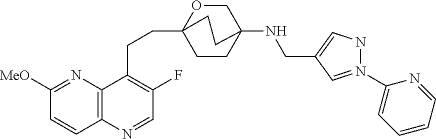

The title compound was prepared from 1-(pyridin-2-yl)-1H-pyrazole-4-carbaldehyde.

¹H NMR (DMSO-d₆): δ 1.59-1.79 (m, 8H), 1.80-1.95 (m, 3H), 3.08-3.16 (m, 2H), 3.61 (s, 4H), 4.03 (s, 3H), 7.22 (d, J=9.2 Hz, 1H), 7.28-7.33 (m, 1H), 7.71 (s, 1H), 7.86-7.89 (m, 1H), 7.91-7.98 (m, 1H), 8.26 (d, J=9.2 Hz, 1H), 8.43 (dd, J=4.9, 1.8 Hz, 1H), 8.47 (s, 1H), 8.74 (s, 1H).

MS (ESI⁺) m/z: 489 (MH⁺).

HRMS (ESI) for C₂₇H₃₀FN₆O₂ (MH⁺): calcd, 489.24143. found, 489.24205.

### Example 75

### 1-(2-(3-Fluoro-6-methoxy-1,5-naphthyridin-4-yl)ethyl)-N-((2-(pyridin-2-yl)thiazol-4-yl)methyl)-2-oxabicyclo[2.2.2]octan-4-amine

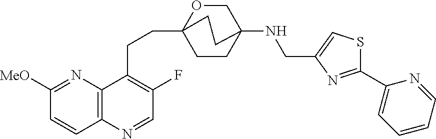 [SMILES: COc1ccc2ncc(F)c(CCC34CCC(NCc5csc(-c6ccccn6)n5)(CC3)CO4)c2n1]

The title compound was prepared from 2-(pyridin-2-yl)-1,3-thiazole-4-carbaldehyde

¹H NMR (DMSO-d₆): δ 1.58-1.81 (m, 8H), 1.83-1.93 (m, 2H), 1.93-2.03 (m, 1H), 3.07-3.17 (m, 2H), 3.63 (s, 2H), 3.85 (d, J=9.2 Hz, 2H), 4.03 (s, 3H), 7.23 (d, J=9.2 Hz, 1H), 7.45-7.48 (m, 1H), 7.52 (s, 1H), 7.94 (td, J=7.8, 1.6 Hz, 1H), 8.07-8.10 (m, 1H), 8.26 (d, J=9.2 Hz, 1H), 8.59-8.61 (m, 1H), 8.75 (s, 1H).

MS (ESI⁺) m/z: 506 (MH⁺).

HRMS (ESI⁺) for C₂₇H₂₉FN₅O₂S (MH⁺): calcd, 506.20260. found, 506.20301.

### Example 76

### 1-(2-(3-Fluoro-6-methoxy-1,5-naphthyridin-4-yl)ethyl)-N-((1-(pyrimidin-2-yl)-1H-imidazol-4-yl)methyl)-2-oxabicyclo[2.2.2]octan-4-amine

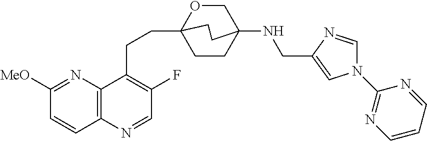 [SMILES: COc1ccc2ncc(F)c(CCC34CCC(NCc5cn(-c6ncccn6)cn5)(CC3)CO4)c2n1]

The title compound was prepared from 1-(pyrimidin-2-yl)-1H-imidazole-4-carbaldehyde ¹H NMR (DMSO-d₆): δ 1.58-1.81 (m, 9H), 1.82-1.95 (m, 2H), 3.07-3.17 (m, 2H), 3.61 (s, 4H), 4.03 (s, 3H), 7.22 (d, J=9.2 Hz, 1H), 7.46 (t, J=4.9 Hz, 1H), 7.74 (s, 1H), 8.26 (d, J=9.2 Hz, 1H), 8.48 (s, 1H), 8.74 (s, 1H), 8.84 (d, J=4.9 Hz, 2H).

MS (ESI⁺) m/z: 490 (MH⁺).

HRMS (ESI⁺) for C₂₆H₂₉FN₇O₂ (MH⁺): calcd, 490.23668. found, 490.23617.

### Example 77

### 1-(2-(3-Fluoro-6-methoxy-1,5-naphthyridin-4-yl)ethyl)-N-(thieno[2,3-b]pyridin-2-ylmethyl)-2-oxabicyclo[2.2.2]octan-4-amine

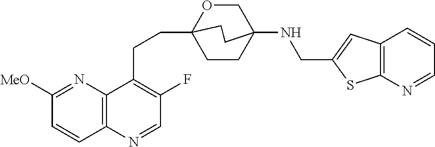 [SMILES: COc1ccc2ncc(F)c(CCC34CCC(NCc5cc6cccnc6s5)(CC3)CO4)c2n1]

The title compound was prepared from thieno[2,3-b]pyridine-2-carbaldehyde.

¹H NMR (DMSO-d₆): δ 1.56-1.80 (m, 8H), 1.80-1.94 (m, 2H), 2.43 (t, J=7.0 Hz, 1H), 3.06-3.15 (m, 2H), 3.62 (s, 2H), 3.98 (d, J=6.7 Hz, 2H), 4.02 (s, 3H), 7.22 (d, J=9.2 Hz, 1H), 7.23 (s, 1H), 7.35 (q, J=4.1 Hz, 1H), 8.09 (dd, J=7.9, 1.2 Hz, 1H), 8.26 (d, J=9.2 Hz, 1H), 8.44 (q, J=2.0 Hz, 1H), 8.74 (s, 1H).

MS (ESI⁺) m/z: 479 (MH⁺).

HRMS (ESI⁺) for C₂₆H₂₈FN₄O₂S (MH⁺): calcd, 479.19170. found, 479.19180.

### Example 78

### 1-(2-(3-Fluoro-6-methoxy-1,5-naphthyridin-4-yl)ethyl)-N-((2-(pyrrolidin-1-yl)pyrimidin-5-yl)methyl)-2-oxabicyclo[2.2.2]octan-4-amine

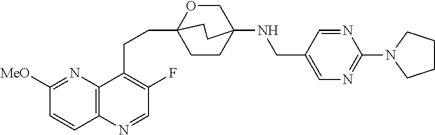 [SMILES: COc1ccc2ncc(F)c(CCC34CCC(NCc5cnc(N6CCCC6)nc5)(CC3)CO4)c2n1]

The title compound was prepared from 2-(pyrrolidin-1-yl)pyrimidine-5-carbaldehyde.

¹H NMR (DMSO-d₆): δ 1.58-1.78 (m, 9H), 1.80-1.93 (m, 6H), 3.06-3.16 (m, 2H), 3.39-3.49 (m, 6H), 3.57 (s, 2H), 4.03 (s, 3H), 7.22 (d, J=8.6 Hz, 1H), 8.22 (s, 2H), 8.26 (d, J=9.2 Hz, 1H), 8.74 (s, 1H).

MS (ESI⁺) m/z: 493 (MH⁺).

HRMS (ESI⁺) for C₂₇H₃₄FN₆O₂ (MH⁺): calcd, 493.27273. found, 493.27202.

### Example 79

### 3-((1-(2-(3-Fluoro-6-methoxy-1,5-naphthyridin-4-yl)ethyl)-2-oxabicyclo[2.2.2]octan-4-ylamino)methyl)-1-methylquinolin-2(1H)-one

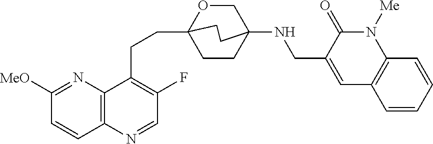 [SMILES: COc1ccc2ncc(F)c(CCC34CCC(NCc5cc6ccccc6n(C)c5=O)(CC3)CO4)c2n1]

The title compound was prepared from 1-methyl-2-oxo-1,2-dihydroquinoline-3-carbaldehyde.

¹H NMR (DMSO-d₆): δ 1.57-1.81 (m, 8H), 1.85-1.93 (m, 2H), 1.97 (brs, 1H), 3.06-3.18 (m, 2H), 3.57 (brs, 2H), 3.63 (brs, 2H), 3.64 (s, 3H), 4.03 (s, 3H), 7.22 (d, J=9.2 Hz, 1H), 7.25 (t, J=7.3 Hz, 1H), 7.51 (t, J=8.6 Hz, 1H), 7.57 (t, J=7.9 Hz, 1H), 7.71 (d, J=7.3 Hz, 1H), 7.88 (s, 1H), 8.26 (d, J=9.2 Hz, 1H), 8.74 (s, 1H).

MS (ESI⁺) m/z: 503 (MH⁺).

HRMS (ESI⁺) for C₂₉H₃₂FN₄O₃ (MH⁺): calcd, 503.24584. found, 503.24601.

### Example 80

### N-((1H-Pyrrolo[2,3-b]pyridin-6-yl)methyl)-1-(2-(3-fluoro-6-methoxy-1,5-naphthyridin-4-yl)ethyl)-2-oxabicyclo[2.2.2]octan-4-amine

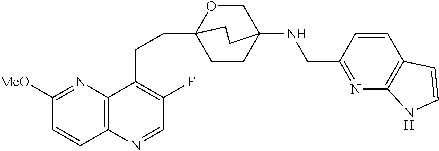 [SMILES: COc1ccc2ncc(F)c(CCC34CCC(NCc5ccc6cc[nH]c6n5)(CC3)CO4)c2n1]

The title compound was prepared from 1H-pyrrolo[2,3-b]pyridine-6-carbaldehyde.

¹H NMR (DMSO-d₆): δ 1.58-1.81 (m, 8H), 1.85-1.98 (m, 3H), 3.07-3.17 (m, 2H), 3.61 (s, 2H), 3.81 (brs, 2H), 4.03 (s, 3H), 6.36-6.40 (m, 1H), 7.10 (d, J=7.9 Hz, 1H), 7.22 (d, J=9.2 Hz, 1H), 7.36 (t, J=3.1 Hz, 1H), 7.86 (d, J=7.9 Hz, 1H), 8.26 (d, J=9.2 Hz, 1H), 8.74 (s, 1H), 11.47 (s, 1H).

MS (ESI) m/z: 462 (MH⁺).

HRMS (ESI) for C₂₆H₂₉FN₅O₂ (MH⁺): calcd, 462.23053. found, 462.23084.

### Example 81

### N-((1H-Pyrrolo[2,3-b]pyridin-5-yl)methyl)-1-(2-(3-fluoro-6-methoxy-1,5-naphthyridin-4-yl)ethyl)-2-oxabicyclo[2.2.2]octan-4-amine

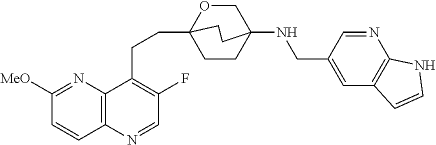 [SMILES: COc1ccc2ncc(F)c(CCC34CCC(NCc5cnc6[nH]ccc6c5)(CC3)CO4)c2n1]

The title compound was prepared from 1H-pyrrolo[2,3-b]pyridine-5-carbaldehyde.

¹H NMR (DMSO-d₆): δ 1.55-1.82 (m, 9H), 1.82-1.94 (m, 2H), 3.07-3.17 (m, 2H), 3.62 (s, 2H), 3.72 (d, J=6.1 Hz, 2H), 4.03 (s, 3H), 6.37 (q, J=1.8 Hz, 1H), 7.22 (d, J=9.2 Hz, 1H), 7.40 (t, J=3.1 Hz, 1H), 7.85 (s, 1H), 8.13 (d, J=1.8 Hz, 1H), 8.26 (d, J=9.2 Hz, 1H), 8.74 (s, 1H), 11.47 (s, 1H).

MS (ESI⁺) m/z: 462 (MH⁺).

HRMS (ESI) for C₂₆H₂₉FN₅O₂ (MH⁺): calcd, 462.23053. found, 462.23037.

### Example 82

### 1-(2-(3-Fluoro-6-methoxy-1,5-naphthyridin-4-yl)ethyl)-N-((6-(pyrrolidin-1-yl)pyridin-3-yl)methyl)-2-oxabicyclo[2.2.2]octan-4-amine

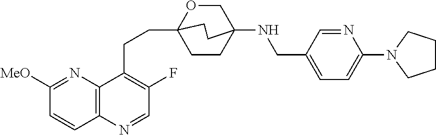 [SMILES: COc1ccc2ncc(F)c(CCC34CCC(NCc5ccc(N6CCCC6)nc5)(CC3)CO4)c2n1]

The title compound was prepared from 6-(pyrrolidin-1-yl)pyridine-3-carbaldehyde.

¹H NMR (DMSO-d₆): δ 1.51-1.77 (m, 9H), 1.77-1.96 (m, 6H), 3.05-3.17 (m, 2H), 3.32 (t, J=6.7 Hz, 4H), 3.48 (brs, 2H), 3.58 (s, 2H), 4.03 (s, 3H), 6.36 (d, J=8.6 Hz, 1H), 7.22 (d, J=9.2 Hz, 1H), 7.41 (dd, J=8.6, 2.4 Hz, 1H), 7.93 (d, J=2.4 Hz, 1H), 8.26 (d, J=9.2 Hz, 1H), 8.74 (s, 1H).

MS (ESI) m/z: 492 (MH⁺).

HRMS (ESI) for C₂₈H₃₅FN₅O₂ (MH⁺): calcd, 492.27748. found, 492.27698.

### Example 83

### 7-((1-(2-(3-Fluoro-6-methoxy-1,5-naphthyridin-4-yl)ethyl)-2-oxabicyclo[2.2.2]octan-4-ylamino)methyl)-1,8-naphthyridin-2(1H)-one

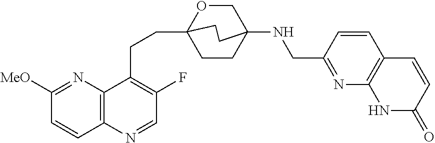 [SMILES: COc1ccc2ncc(F)c(CCC34CCC(NCc5ccc6ccc(=O)[nH]c6n5)(CC3)CO4)c2n1]

The title compound was prepared from 7-oxo-7,8-dihydro-1,8-naphthyridine-2-carbaldehyde.

¹H NMR (DMSO-d₆): δ 1.56-1.79 (m, 8H), 1.79-1.94 (m, 2H), 2.16 (brs, 1H), 3.02-3.19 (m, 2H), 3.60 (s, 2H), 3.80 (d, J=5.5 Hz, 2H), 4.03 (s, 3H), 6.49 (d, J=9.2 Hz, 1H), 7.22 (d, J=9.2 Hz, 1H), 7.33 (d, J=7.9 Hz, 1H), 7.87 (d, J=9.8 Hz, 1H), 8.03 (d, J=7.9 Hz, 1H), 8.26 (d, J=8.6 Hz, 1H), 8.74 (s, 1H), 12.00 (s, 1H).

MS (ESI⁺) m/z: 490 (MH⁺).

HRMS (ESI) for C₂₇H₂₉FN₅O₃ (MH⁺): calcd, 490.22544. found, 490.22620.

### Example 84

### 1-(2-(3-Fluoro-6-methoxy-1,5-naphthyridin-4-yl)ethyl)-N-((6-(piperidin-1-yl)pyridin-3-yl)methyl)-2-oxabicyclo[2.2.2]octan-4-amine

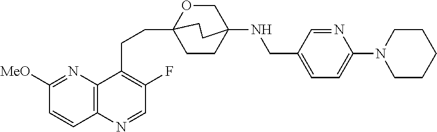 [SMILES: COc1ccc2ncc(F)c(CCC34CCC(NCc5ccc(N6CCCCC6)nc5)(CC3)CO4)c2n1]

The title compound was prepared from 6-(piperidin-1-yl)pyridine-3-carbaldehyde.

¹H NMR (DMSO-d₆): δ 1.49-1.79 (m, 15H), 1.79-1.93 (m, 2H), 3.07-3.16 (m, 2H), 3.39-3.53 (m, 6H), 3.57 (s, 2H), 4.03 (s, 3H), 6.73 (d, J=9.2 Hz, 1H), 7.22 (d, J=9.2 Hz, 1H), 7.43 (dd, J=8.9, 2.1 Hz, 1H), 7.97 (d, J=2.4 Hz, 1H), 8.26 (d, J=9.2 Hz, 1H), 8.74 (s, 1H).

MS (ESI⁺) m/z: 506 (MH⁺).

HRMS (ESI⁺) for C₂₉H₃₇FN₅O₂ (MH⁺): calcd, 506.29313. found, 506.29301.

### Example 85

### 5-((1-(2-(3-Fluoro-6-methoxy-1,5-naphthyridin-4-yl)ethyl)-2-oxabicyclo[2.2.2]octan-4-ylamino)methyl)-N,N-dimethylpyrimidin-2-amine

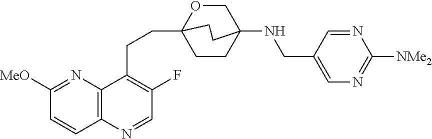 [SMILES: COc1ccc2ncc(F)c(CCC34CCC(NCc5cnc(N(C)C)nc5)(CC3)CO4)c2n1]

The title compound was prepared from 2-(dimethylamino)pyrimidine-5-carbaldehyde.

¹H NMR (DMSO-d₆): δ 1.55-1.93 (m, 11H), 3.07-3.18 (m, 8H), 3.45 (brs, 2H), 3.57 (s, 2H), 4.03 (s, 3H), 7.22 (d, J=9.2 Hz, 1H), 8.21-8.29 (m, 3H), 8.74 (s, 1H).

MS (ESI⁺) m/z: 467 (MH⁺).

HRMS (ESI⁺) for C₂₅H₃₂FN₆O₂ (MH⁺): calcd, 467.25708. found, 467.25610.

### Example 86

Ethyl 2-(((1-(2-(3-Fluoro-6-methoxy-1,5-naphthyridin-4-yl)ethyl)-2-oxabicyclo[2.2.2]octan-4-yl)amino)methyl)thiazole-4-carboxylate

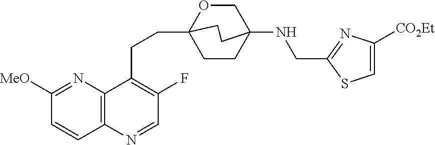 [SMILES: CCOC(=O)c1csc(CNC23CCC(CCc4c(F)cnc5ccc(OC)nc45)(CC2)OC3)n1]

The title compound was prepared from ethyl 2-formyl-1,3-thiazole-4-carboxylate.

¹H NMR (DMSO-d₆): δ 1.28 (t, J=7.3 Hz, 3H), 1.62-1.75 (m, 8H), 1.83-1.89 (m, 2H), 2.87 (t, J=7.3 Hz, 1H), 3.08-3.13 (m, 2H), 3.60 (s, 2H), 3.96 (d, J=7.3 Hz, 2H), 4.04 (s, 3H), 4.27 (q, J=7.3 Hz, 2H), 7.22 (d, J=9.2 Hz, 1H), 8.26 (d, J=8.6 Hz, 1H), 8.36 (s, 1H), 8.74 (s, 1H).

MS (ESI⁺) m/z: 501 (MH⁺).

HRMS (ESI⁺) for C₂₅H₃₀FN₄O₄S (MH⁺): calcd, 501.19718. found, 501.19762.

### Example 87

### 1-(2-(3-Fluoro-6-methoxy-1,5-naphthyridin-4-yl)ethyl)-N-((6-fluorobenzo[d][1,3]dioxol-5-yl)methyl)-2-oxabicyclo[2.2.2]octan-4-amine Hydrochloride

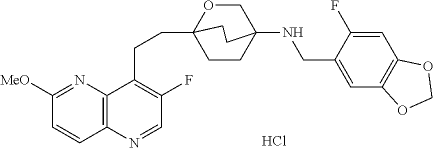 [SMILES: COc1ccc2ncc(F)c(CCC34CCC(NCc5cc6c(cc5F)OCO6)(CC3)CO4)c2n1 ; Cl]

The title compound was prepared from 6-fluoro-1,3-benzodioxole-5-carbaldehyde.

¹H NMR (DMSO-d₆): δ 1.68-1.73 (m, 2H), 1.84-1.87 (m, 2H), 1.99-2.06 (m, 6H), 3.11-3.15 (m, 2H), 3.91 (s, 2H), 4.04 (s, 5H), 6.10 (s, 2H), 7.07 (d, J=9.8 Hz, 1H), 7.19 (d, J=5.5 Hz, 1H), 7.23 (d, J=9.2 Hz, 1H), 8.27 (d, J=9.2 Hz, 1H), 8.76 (s, 1H), 9.29 (br, 2H).

MS (ESI⁺) m/z: 484 (MH⁺) (as free base).

HRMS (ESI⁺) for C₂₆H₂₈F₂N₃O₄ (MH⁺) (as free base): calcd, 484.20479. found, 484.20413.

### Example 88

Free Base: 7-chloro-6-(((1-(2-(3-fluoro-6-methoxy-1,5-naphthyridin-4-yl)ethyl)-2-oxabicyclo[2.2.2]octan-4-yl)amino)methyl)-2H-pyrido[3,2-b][1,4]oxazin-3(4H)-one

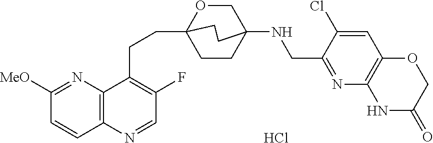 [SMILES: COc1ccc2ncc(F)c(CCC34CCC(NCc5nc6c(cc5Cl)OCC(=O)N6)(CC3)CO4)c2n1 ; Cl]

The title compound was prepared from AE.

¹H NMR (DMSO-d₆): δ 1.59-1.77 (m, 9H), 1.82-1.94 (m, 2H), 3.06-3.16 (m, 2H), 3.58 (s, 2H), 3.72 (d, J=6.7 Hz, 2H), 4.03 (s, 3H), 4.65 (s, 2H), 7.22 (d, J=8.6 Hz, 1H), 7.51 (s, 1H), 8.26 (d, J=9.2 Hz, 1H), 8.74 (s, 1H), 11.37 (s, 1H).

MS (ESI⁺) m/z: 528 (MH⁺).

HRMS (ESI⁺) for C₂₆H₂₈ClFN₅O₄ (MH⁺): calcd, 528.18138. found, 528.18163.

HCl salt: 7-chloro-6-(((1-(2-(3-fluoro-6-methoxy-1,5-naphthyridin-4-yl)ethyl)-2-oxabicyclo[2.2.2]octan-4-yl)amino)methyl)-2H-pyrido[3,2-b][1,4]oxazin-3(4H)-one hydrochloride

¹H NMR (DMSO-d₆): δ 1.65-1.76 (m, 2H), 1.80-1.92 (m, 2H), 1.93-2.13 (m, 6H), 3.07-3.19 (m, 2H), 3.93 (s, 2H), 4.04 (s, 3H), 4.20 (br, 2H), 4.75 (s, 2H), 7.24 (d, J=9.2 Hz, 1H), 7.73 (s, 1H), 8.28 (d, J=9.2 Hz, 1H), 8.76 (s, 1H), 9.24 (br, 2H), 11.53 (s, 1H).

MS (ESI) m/z: 528 (MH⁺) (as free base).

HRMS (ESI⁺) for C₂₆H₂₈ClFN₅O₄ (MH⁺) (as free base): calcd, 528.18138. found, 528.18093.

### Example 89

### 1-(2-(3-Fluoro-6-methoxy-1,5-naphthyridin-4-yl)ethyl)-N-((4-methyl-3,4-dihydro-2H-pyrido[3,2-b][1,4]oxazin-7-yl)methyl)-2-oxabicyclo[2.2.2]octan-4-amine

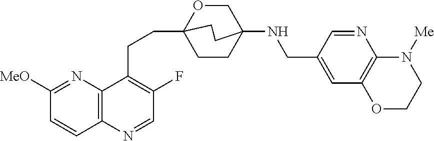 [SMILES: COc1ccc2ncc(F)c(CCC34CCC(NCc5cnc6c(c5)OCCN6C)(CC3)CO4)c2n1]

The title compound was prepared from 4-methyl-3,4-dihydro-2H-pyrido[3,2-b][1,4]oxazine-7-carbaldehyde.

¹H NMR (DMSO-d₆): δ 1.57-1.91 (m, 10H), 2.96 (s, 3H), 3.07-3.15 (m, 2H), 3.36 (t, J=4.6 Hz, 2H), 3.45 (brs, 2H), 3.56 (s, 2H), 4.02 (s, 3H), 4.18 (t, J=4.6 Hz, 2H), 6.87 (d, J=1.8 Hz, 1H), 7.22 (d, J=9.2 Hz, 1H), 7.55 (d, J=1.8 Hz, 1H), 8.26 (d, J=9.2 Hz, 1H), 8.74 (s, 1H).

MS (ESI) m/z: 494 (MH⁺).

HRMS (ESI) for C₂₇H₃₃FN₅O₃ (MH⁺): calcd, 494.25674. found, 494.25692.

### Example 90

### N-((2,2-Difluorobenzo[d][1,3]dioxol-5-yl)methyl)-1-(2-(3-fluoro-6-methoxy-1,5-naphthyridin-4-yl)ethyl)-2-oxabicyclo[2.2.2]octan-4-amine

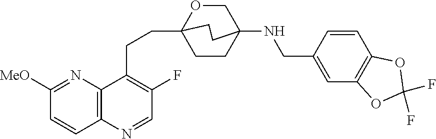 [SMILES: COc1ccc2ncc(F)c(CCC34CCC(NCc5ccc6c(c5)OC(F)(F)O6)(CC3)CO4)c2n1]

Prepared from 2,2-difluoro-1,3-benzodioxole-5-carbaldehyde

¹H NMR (DMSO-d₆): δ 1.56-1.93 (m, 11H), 3.05-3.16 (m, 2H), 3.58 (s, 2H), 3.66 (s, 2H), 4.03 (s, 3H), 7.15 (d, J=7.9 Hz, 1H), 7.22 (d, J=9.2 Hz, 1H), 7.29 (d, J=8.6 Hz, 1H), 7.35 (s, 1H), 8.26 (d, J=8.6 Hz, 1H), 8.74 (s, 1H).

MS (ESI⁺) m/z: 502 (MH⁺).

HRMS (ESI⁺) for C₂₆H₂₇F₃N₃O₄ (MH⁺): calcd, 502.19537. found, 502.19456.

### Example 91

### 5-((1-(2-(3-Fluoro-6-methoxy-1,5-naphthyridin-4-yl)ethyl)-2-oxabicyclo[2.2.2]octan-4-ylamino)methyl)-1,3-dimethyl-1H-benzo[d]imidazol-2(3H)-one

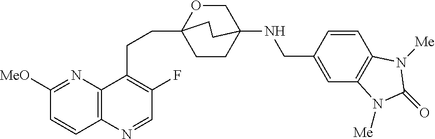 [SMILES: COc1ccc2ncc(F)c(CCC34CCC(NCc5ccc6c(c5)n(C)c(=O)n6C)(CC3)CO4)c2n1]

The title compound was prepared from 1,3-dimethyl-2-oxo-2,3-dihydro-1H-benzimidazole-5-carbaldehyde.

¹H NMR (DMSO-d₆): δ 1.62-1.90 (m, 11H), 3.08-3.15 (m, 2H), 3.29 (s, 6H), 3.60 (s, 2H), 3.67 (s, 2H), 4.03 (s, 3H), 7.02 (s, 2H), 7.09 (s, 1H), 7.22 (d, J=9.2 Hz, 1H), 8.26 (d, J=9.2 Hz, 1H), 8.74 (s, 1H).

MS (ESI⁺) m/z: 506 (MH⁺).

HRMS (ESI⁺) for C₂₈H₃₃FN₅O₃ (MH⁺): calcd, 506.25674. found, 506.25682.

### Example 92

### N-((1-(2,2-Difluoroethyl)-1H-pyrazol-4-yl)methyl)-1-(2-(3-fluoro-6-methoxy-1,5-naphthyridin-4-yl)ethyl)-2-oxabicyclo[2.2.2]octan-4-amine

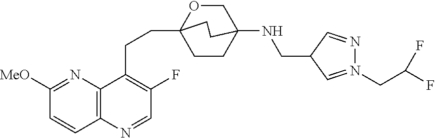

The title compound was prepared from 1-(2,2-difluoroethyl)-1H-pyrazole-4-carbaldehyde.

¹H NMR (CDCl₃): δ 1.58-1.90 (m, 11H), 3.09-3.14 (m, 2H), 3.50 (s, 2H), 3.58 (s, 2H), 4.03 (s, 3H), 4.53 (dt, J=15.3, 3.7 Hz, 2H), 6.28 (tt, J=55.0, 4.3 Hz, 1H), 7.22 (d, J=9.2 Hz, 1H), 7.39 (s, 1H), 7.61 (s, 1H), 8.25 (d, J=9.2 Hz, 1H), 8.74 (s, 1H).

MS (ESI⁺) m/z: 476 (MH⁺).

HRMS (ESI⁺) for C₂₄H₂₉F₃N₅O₂ (MH⁺): calcd, 476.22733. found, 476.22810.

### Example 93

### N-((4-Chloro-1-methyl-1H-pyrazol-3-yl)methyl)-1-(2-(3-fluoro-6-methoxy-1,5-naphthyridin-4-yl)ethyl)-2-oxabicyclo[2.2.2]octan-4-amine

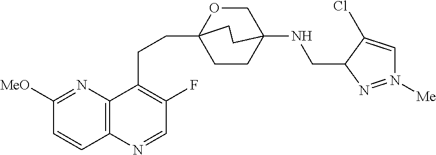

The title compound was prepared from 4-chloro-1-methyl-1H-pyrazole-3-carbaldehyde.

¹H NMR (CDCl₃): δ 1.40-1.94 (m, 11H), 3.07-3.13 (m, 2H), 3.56 (s, 2H), 3.58 (s, 2H), 3.75 (s, 3H), 4.03 (s, 3H), 7.22 (d, J=9.2 Hz, 1H), 7.83 (s, 1H), 8.26 (d, J=9.2 Hz, 1H), 8.74 (s, 1H).

MS (ESI⁺) m/z: 460 (MH⁺).

HRMS (ESI⁺) for C₂₃H₂₈ClFN₅O₂ (MH⁺): calcd, 460.19156. found, 460.19192.

### Example 94

### 5-((1-(2-(3-Fluoro-6-methoxy-1,5-naphthyridin-4-yl)ethyl)-2-oxabicyclo[2.2.2]octan-4-ylamino)methyl)-1,3-dimethylpyrimidine-2,4(1H,3H)-dione

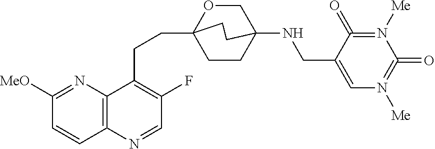

The title compound was prepared from 1,3-dimethyl-2,4-dioxo-1,2,3,4-tetrahydropyrimidine-5-carbaldehyde.

¹H NMR (CDCl₃): δ 1.58-1.92 (m, 11H), 3.07-3.13 (m, 2H), 3.16 (s, 3H), 3.29-3.31 (m, 5H), 3.57 (s, 2H), 4.03 (s, 3H), 7.22 (d, J=9.2 Hz, 1H), 7.83 (s, 1H), 8.26 (d, J=9.2 Hz, 1H), 8.74 (s, 1H).

MS (ESI⁺) m/z: 484 (MH⁺).

HRMS (ESI⁺) for C₂₅H₃₁FN₅O₄ (MH⁺): calcd, 484.23601. found, 484.23549.

### Example 95

### N-(4-(Difluoromethoxy)-3-methoxybenzyl)-1-(2-(3-fluoro-6-methoxy-1,5-naphthyridin-4-yl)ethyl)-2-oxabicyclo[2.2.2]octan-4-amine

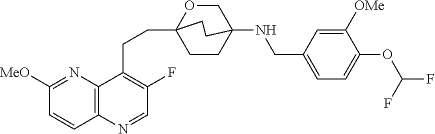 [SMILES: COc1ccc2ncc(F)c(CCC34CCC(NCc5ccc(OC(F)F)c(OC)c5)(CC3)CO4)c2n1]

The title compound was prepared from 4-(difluoromethoxy)-3-methoxybenzaldehyde.

¹H NMR (DMSO-d₆): δ 1.58-1.93 (m, 11H), 3.07-3.15 (m, 2H), 3.59 (s, 2H), 3.63 (d, J=6.1 Hz, 2H), 3.80 (s, 3H), 4.03 (s, 3H), 6.90 (d, J=8.6 Hz, 1H), 6.97 (t, J=75.2 Hz, 1H), 7.06 (d, J=7.9 Hz, 1H), 7.10 (s, 1H), 7.22 (d, J=9.2 Hz, 1H), 8.26 (d, J=9.2 Hz, 1H), 8.74 (s, 1H).

MS (ESI⁺) m/z: 518 (MH⁺).

HRMS (ESI⁺) for C₂₇H₃₁F₃N₃O₄ (MH⁺): calcd, 518.22667. found, 518.22672.

### Example 96

### 7-(((1-(2-(3-Fluoro-6-methoxy-1,5-naphthyridin-4-yl)ethyl)-2-oxabicyclo[2.2.2]octan-4-yl)amino)methyl)-1H-pyrido[3,4-b][1,4]oxazin-2(3H)-one

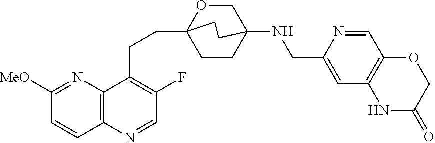 [SMILES: COc1ccc2ncc(F)c(CCC34CCC(NCc5cc6c(cn5)OCC(=O)N6)(CC3)CO4)c2n1]

The title compound was prepared from 2-oxo-2,3-dihydro-1H-pyrido[3,4-b][1,4]oxazine-7-carbaldehyde.

¹H NMR (DMSO-d₆): δ 1.58-1.77 (m, 9H), 1.81-1.92 (m, 2H), 3.07-3.16 (m, 2H), 3.58 (s, 2H), 3.64 (s, 2H), 4.02 (s, 3H), 4.63 (s, 2H), 6.97 (s, 1H), 7.22 (d, J=9.2 Hz, 1H), 8.03 (s, 1H), 8.26 (d, J=9.2 Hz, 1H), 8.74 (s, 1H), 10.99 (s, 1H).

MS (ESI⁺) m/z: 494 (MH⁺).

HRMS (ESI⁺) for C₂₆H₂₉FN₅O₄ (MH⁺): calcd, 494.22036. found, 494.22099.

### Example 97

### 3-(((1-(2-(3-Fluoro-6-methoxy-1,5-naphthyridin-4-yl)ethyl)-2-oxabicyclo[2.2.2]octan-4-yl)amino)methyl)quinoxalin-2(1H)-one

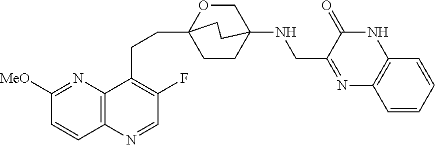 [SMILES: COc1ccc2ncc(F)c(CCC34CCC(NCc5nc6ccccc6[nH]c5=O)(CC3)CO4)c2n1]

The title compound was prepared from 3-oxo-3,4-dihydroquinoxaline-2-carbaldehyde ¹H NMR (DMSO-d₆): δ 1.55-1.76 (m, 9H), 1.82-1.91 (m, 2H), 3.05-3.18 (m, 2H), 3.63 (s, 2H), 3.85 (s, 2H), 4.03 (s, 3H), 7.23 (d, J=9.2 Hz, 1H), 7.27-7.31 (m, 2H), 7.48-7.51 (m, 1H), 7.76 (d, J=8.6 Hz, 1H), 8.26 (d, J=9.2 Hz, 1H), 8.75 (s, 1H), 12.40 (br, 1H).

MS (ESI⁺) m/z: 490 (MH⁺).

HRMS (ESI⁺) for C₂₇H₂₉FN₅O₃ (MH⁺): calcd, 490.22544. found, 490.22554.

### Example 98

### 7-Fluoro-6-(((1-(2-(3-fluoro-6-methoxy-1,5-naphthyridin-4-yl)ethyl)-2-oxabicyclo[2.2.2]octan-4-yl)amino)methyl)-2H-pyrido[3,2-b][1,4]oxazin-3(4H)-one

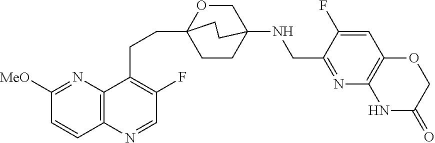 [SMILES: COc1ccc2ncc(F)c(CCC34CCC(NCc5nc6c(cc5F)OCC(=O)N6)(CC3)CO4)c2n1]

The title compound was prepared from AF.

¹H NMR (DMSO-d₆): δ 1.62-1.73 (m, 9H), 1.83-1.90 (m, 2H), 3.08-3.13 (m, 2H), 3.57 (s, 2H), 3.66 (d, J=4.9 Hz, 2H), 4.03 (s, 3H), 4.63 (s, 2H), 7.22 (d, J=9.2 Hz, 1H), 7.41 (d, J=9.8 Hz, 1H), 8.26 (d, J=9.2 Hz, 1H), 8.74 (s, 1H), 11.28 (br, 1H).

MS (ESI⁺) m/z: 512 (MH⁺).

HRMS (ESI⁺) for C₂₆H₂₈F₂N₅O₄ (MH⁺): calcd, 512.21093. found, 512.21034.

### Example 99

### 6-((1-(2-(3-Fluoro-6-methoxy-1,5-naphthyridin-4-yl)ethyl)-2-oxabicyclo[2.2.2]octan-4-ylamino)methyl)-2,2-dimethyl-2H-pyrido[3,2-b][1,4]oxazin-3(4H)-one

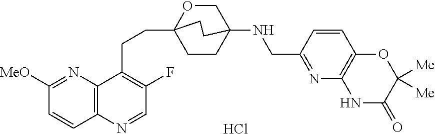 [SMILES: COc1ccc2ncc(F)c(CCC34CCC(NCc5ccc6c(n5)NC(=O)C(C)(C)O6)(CC3)CO4)c2n1 ; Cl]

The title compound was prepared from AG.

Free base: ¹H NMR (DMSO-d₆): δ 1.38 (s, 6H), 1.60-1.74 (m, 8H), 1.81-1.95 (m, 3H), 3.06-3.18 (m, 2H), 3.58 (s, 2H), 3.63 (brs, 3H), 4.04 (s, 3H), 7.01 (d, J=7.9 Hz, 1H), 7.23 (d, J=9.1 Hz, 1H), 7.28 (d, J=8.5 Hz, 1H), 8.26 (d, J=9.1 Hz, 1H), 8.74 (s, 1H), 11.08 (brs, 1H).

MS (ESI⁺) m/z: 522 (MH⁺).

HRMS (ESI⁺) for C₂₈H₃₃FN₅O₄ (MH⁺): calcd, 522.25166. found, 522.25131.

HCl salt: ¹H NMR (DMSO-d₆): δ 1.42 (s, 6H), 1.66-1.75 (m, 2H), 1.79-1.92 (m, 2H), 1.93-2.10 (m, 6H), 3.08-3.17 (m, 2H), 3.92 (s, 2H), 4.04 (s, 3H), 4.07-4.16 (m, 2H), 7.24 (d, J=9.2 Hz, 1H), 7.24 (d, J=8.0 Hz, 1H), 7.46 (d, J=8.6 Hz, 1H), 8.27 (d, J=9.2 Hz, 1H), 8.76 (s, 1H), 9.31 (brs, 2H), 11.28 (s, 1H).

MS (ESI⁺) m/z: 522 (MH⁺) (as free base).

HRMS (ESI⁺) for C₂₈H₃₃FN₅O₄ (MH⁺) (as free base): calcd, 522.25166. found, 522.25195.

### Example 100

### 6-((1-(2-(3-Fluoro-6-methoxy-1,5-naphthyridin-4-yl)ethyl)-2-oxabicyclo[2.2.2]octan-4-ylamino)methyl)-2-methyl-2H-pyrido[3,2-b][1,4]oxazin-3(4H)-one Hydrochloride (Enantiomer A and Enantiomer B)

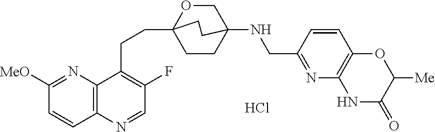 [SMILES: COc1ccc2ncc(F)c(CCC34CCC(NCc5ccc6c(n5)NC(=O)C(C)O6)(CC3)CO4)c2n1 ; Cl]

The title compound was prepared from AH.

Optical resolution (CHIRALPAK IA, hexane:ethanol=20:80) of the racemate gave Enantiomer A and Enantiomer B.

Free base of Enantiomer A: ¹H NMR (DMSO-d₆): δ 1.41 (d, J=7.3 Hz, 3H), 1.57-1.78 (m, 8H), 1.79-1.96 (m, 3H), 3.06-3.17 (m, 2H), 3.58 (s, 2H), 3.62 (s, 2H), 4.03 (s, 3H), 4.69 (q, J=7.3 Hz, 1H), 7.01 (d, J=8.0 Hz, 1H), 7.22 (d, J=8.6 Hz, 1H), 7.29 (d, J=8.6 Hz, 1H), 8.26 (d, J=9.2 Hz, 1H), 8.74 (s, 1H), 11.11 (s, 1H).

MS (ESI⁺) m/z: 508 (MH⁺).

HRMS (ESI⁺) for C₂₇H₃₁FN₅O₄ (MH⁺): calcd, 508.23601. found, 508.23606.

HCl salt of Enantiomer A: ¹H NMR (DMSO-d₆): δ 1.44 (d, J=6.7 Hz, 3H), 1.65-1.75 (m, 2H), 1.78-1.91 (m, 2H), 1.93-2.10 (m, 6H), 3.08-3.17 (m, 2H), 3.91 (s, 2H), 4.04 (s, 3H), 4.07-4.16 (m, 2H), 4.79 (q, J=6.7 Hz, 1H), 7.23 (d, J=8.0 Hz, 1H), 7.24 (d, J=8.6 Hz, 1H), 7.48 (d, J=8.6 Hz, 1H), 8.27 (d, J=9.2 Hz, 1H), 8.76 (s, 1H), 9.30 (brs, 2H), 11.30 (s, 1H).

MS (ESI⁺) m/z: 508 (MH⁺) (as free base).

HRMS (ESI⁺) for C₂₇H₃₁FN₅O₄ (MH⁺) (as free base): calcd, 508.23601. found, 508.23511.

Free base of Enantiomer B: ¹H NMR (DMSO-d₆): δ 1.41 (d, J=6.7 Hz, 3H), 1.56-1.77 (m, 8H), 1.79-1.95 (m, 3H), 3.06-3.16 (m, 2H), 3.58 (s, 2H), 3.62 (s, 2H), 4.02 (s, 3H), 4.69 (q, J=6.7 Hz, 1H), 7.01 (d, J=8.0 Hz, 1H), 7.22 (d, J=8.6 Hz, 1H), 7.29 (d, J=8.0 Hz, 1H), 8.26 (d, J=9.2 Hz, 1H), 8.74 (s, 1H), 11.11 (s, 1H).

MS (ESI⁺) m/z: 508 (MH⁺).

HRMS (ESI⁺) for C₂₇H₃₁FN₅O₄ (MH⁺): calcd, 508.23601. found, 508.23559.

HCl salt of Enantiomer B: ¹H NMR (DMSO-d₆): δ 1.44 (d, J=6.7 Hz, 3H), 1.65-1.75 (m, 2H), 1.78-1.91 (m, 2H), 1.93-2.11 (m, 6H), 3.08-3.17 (m, 2H), 3.92 (s, 2H), 4.04 (s, 3H), 4.07-4.15 (m, 2H), 4.80 (q, J=6.7 Hz, 1H), 7.24 (d, J=9.2 Hz, 1H), 7.25 (d, J=8.6 Hz, 1H), 7.47 (d, J=8.6 Hz, 1H), 8.27 (d, J=8.6 Hz, 1H), 8.76 (s, 1H), 9.36 (brs, 2H), 11.30 (s, 1H).

MS (ESI⁺) m/z: 508 (MH⁺) (as free base).

HRMS (ESI⁺) for C₂₇H₃₁FN₅O₄ (MH⁺) (as free base): calcd, 508.23601. found, 508.23573.

### Example 101

### 6-((1-(2-(3-Fluoro-6-methoxy-1,5-naphthyridin-4-yl)ethyl)-2-oxabicyclo[2.2.2]octan-4-ylamino)methyl)-4-methyl-2H-pyrido[3,2-b][1,4]oxazin-3(4H)-one

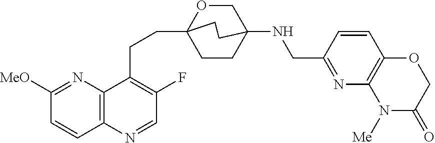 [SMILES: COc1ccc2ncc(F)c(CCC34CCC(NCc5ccc6c(n5)N(C)C(=O)CO6)(CC3)CO4)c2n1]

The title compound was prepared from AK.

¹H NMR (DMSO-d₆): δ 1.60-1.77 (m, 8H), 1.81-2.03 (m, 2H), 1.95-2.03 (m, 1H), 3.08-3.15 (m, 2H), 3.32 (s, 3H), 3.59 (s, 2H), 3.69 (s, 2H), 4.03 (s, 3H), 4.71 (s, 2H), 7.07 (d, J=8.6 Hz, 1H), 7.22 (d, J=9.2 Hz, 1H), 7.32 (d, J=8.0 Hz, 1H), 8.26 (d, J=9.2 Hz, 1H), 8.74 (s, 1H).

MS (ESI⁺) m/z: 508 (MH⁺).

HRMS (ESI⁺) for C₂₇H₃₁FN₅O₄ (MH⁺): calcd, 508.23601. found, 508.23662.

### Example 102

### 6-Fluoro-3-((1-(2-(3-fluoro-6-methoxy-1,5-naphthyridin-4-yl)ethyl)-2-oxabicyclo[2.2.2]octan-4-ylamino)methyl)-1-methylquinolin-2(1H)-one

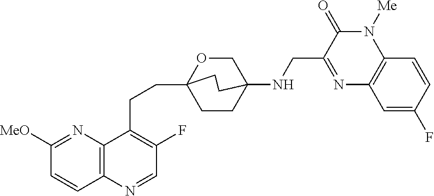 [SMILES: COc1ccc2ncc(F)c(CCC34CCC(NCc5nc6cc(F)ccc6n(C)c5=O)(CC3)CO4)c2n1]

The title compound was prepared from 6-fluoro-1-methyl-2-oxo-1,2-dihydroquinoline-3-carbaldehyde.

¹H NMR (DMSO-d₆): δ 1.60-1.80 (m, 8H), 1.82-2.03 (m, 3H), 3.07-3.18 (m, 2H), 3.57 (s, 2H), 3.63 (s, 2H), 3.64 (s, 3H), 4.03 (s, 3H), 7.23 (d, J=9.2 Hz, 1H), 7.45 (dt, J=9.2, 3.0 Hz, 1H), 7.55 (dd, J=9.2, 4.3 Hz, 1H), 7.62 (dd, J=9.2, 2.4 Hz, 1H), 7.88 (s, 1H), 8.26 (d, J=8.6 Hz, 1H), 8.74 (s, 1H).

MS (ESI⁺) m/z: 521 (MH⁺).

HRMS (ESI⁺) for C₂₉H₃₁F₂N₄O₃ (MH⁺): calcd, 521.23642. found, 521.23582.

### Example 103

### 3-((1-(2-(3-Fluoro-6-methoxy-1,5-naphthyridin-4-yl)ethyl)-2-oxabicyclo[2.2.2]octan-4-ylamino)methyl)-4-methoxy-1-methylquinolin-2(1H)-one

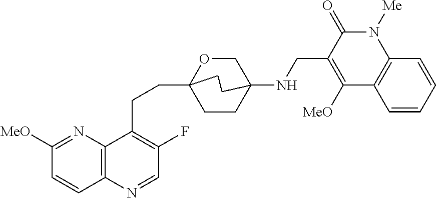 [SMILES: COc1ccc2ncc(F)c(CCC34CCC(NCc5c(OC)c6ccccc6n(C)c5=O)(CC3)CO4)c2n1]

The title compound was prepared from 4-methoxy-1-methyl-2-oxo-1,2-dihydroquinoline-3-carbaldehyde (AL).

¹H NMR (DMSO-d₆): δ 1.60-1.80 (m, 9H), 1.83-1.98 (m, 2H), 3.08-3.15 (m, 2H), 3.58-3.64 (m, 4H), 3.61 (s, 3H), 3.96 (s, 3H), 4.03 (s, 3H), 7.22 (d, J=8.6 Hz, 1H), 7.31 (t, J=7.9 Hz, 1H), 7.56 (d, J=7.9 Hz, 1H), 7.64 (m, 1H), 7.82 (dd, J=7.9, 1.2 Hz, 1H), 8.26 (d, J=9.2 Hz, 1H), 8.74 (s, 1H).

MS (ESI⁺) m/z: 533 (MH⁺).

HRMS (ESI⁺) for C₃₀H₃₄FN₄O₄ (MH⁺): calcd, 533.25641. found, 533.25625.

### Example 104

### 7-Chloro-3-((1-(2-(3-fluoro-6-methoxy-1,5-naphthyridin-4-yl)ethyl)-2-oxabicyclo[2.2.2]octan-4-ylamino)methyl)-1-methylquinolin-2(1H)-one

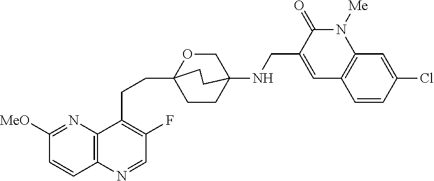 [SMILES: COc1ccc2ncc(F)c(CCC34CCC(NCc5cc6ccc(Cl)cc6n(C)c5=O)(CC3)CO4)c2n1]

The title compound was prepared from 7-chloro-1-methyl-2-oxo-1,2-dihydroquinoline-3-carbaldehyde.

¹H NMR (DMSO-d₆): δ 1.60-1.80 (m, 8H), 1.82-2.02 (m, 3H), 3.07-3.15 (m, 2H), 3.55 (s, 2H), 3.62 (m, 5H), 4.03 (s, 3H), 7.22 (d, J=9.2 Hz, 1H), 7.29 (dd, J=8.6, 1.8 Hz, 1H), 7.59 (d, J=1.8 Hz, 1H), 7.74 (d, J=8.0 Hz, 1H), 7.89 (s, 1H), 8.26 (d, J=8.6 Hz, 1H), 8.74 (s, 1H).

MS (ESI) m/z: 537 (MH⁺).

HRMS (ESI) for C₂₉H₃₁ClFN₄O₃ (MH⁺): calcd, 537.20687. found, 537.20605.

### Example 105

### 7-Fluoro-3-((1-(2-(3-fluoro-6-methoxy-1,5-naphthyridin-4-yl)ethyl)-2-oxabicyclo[2.2.2]octan-4-ylamino)methyl)-1-methylquinolin-2(1H)-one

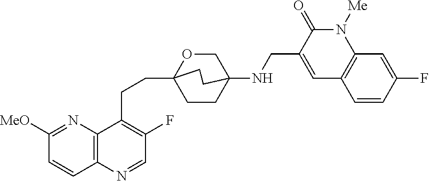 [SMILES: COc1ccc2ncc(F)c(CCC34CCC(NCc5cc6ccc(F)cc6n(C)c5=O)(CC3)CO4)c2n1]

The title compound was prepared from 7-fluoro-1-methyl-2-oxo-1,2-dihydroquinoline-3-carbaldehyde.

¹H NMR (DMSO-d₆): δ 1.60-1.79 (m, 8H), 1.84-1.99 (m, 3H), 3.08-3.16 (m, 2H), 3.55 (d, J=4.9 Hz, 2H), 3.60 (s, 3H), 3.62 (s, 2H), 4.03 (s, 3H), 7.12 (td, J=11.0, 8.6, 2.4 Hz, 1H), 7.22 (d, J=8.6 Hz, 1H), 7.39 (dd, J=11.6, 2.4 Hz, 1H), 7.78 (dd, J=8.6, 6.8 Hz, 1H), 7.89 (s, 1H), 8.26 (d, J=9.2 Hz, 1H), 8.75 (s, 1H).

MS (ESI⁺) m/z: 521 (MH⁺).

HRMS (ESI) for C₂₉H₃₁F₂N₄O₃ (MH⁺): calcd, 521.23642. found, 521.23584.

### Example 106

### 5-Chloro-3-((1-(2-(3-fluoro-6-methoxy-1,5-naphthyridin-4-yl)ethyl)-2-oxabicyclo[2.2.2]octan-4-ylamino)methyl)-1-methylquinolin-2(1H)-one

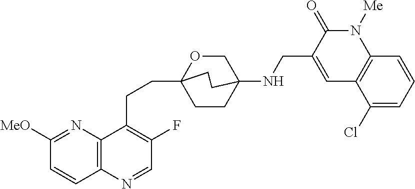 [SMILES: COc1ccc2ncc(F)c(CCC34CCC(NCc5cc6c(Cl)cccc6n(C)c5=O)(CC3)CO4)c2n1]

The title compound was prepared from 5-chloro-1-methyl-2-oxo-1,2-dihydroquinoline-3-carbaldehyde (AM).

¹H NMR (DMSO-d₆): δ 1.60-1.80 (m, 8H), 1.82-2.32 (m, 3H), 3.07-3.15 (m, 2H), 3.55-3.64 (m, 4H), 3.66 (m, 3H), 4.03 (s, 3H), 7.22 (d, J=9.2 Hz, 1H), 7.41 (dd, J=7.4, 1.8 Hz, 1H), 7.52-7.60 (m, 2H), 8.20 (m, 1H), 8.27 (d, J=9.2 Hz, 1H), 8.74 (s, 1H).

MS (ESI⁺) m/z: 537 (MH⁺).

HRMS (ESI⁺) for C₂₉H₃₁ClFN₄O₃ (MH⁺): calcd, 537.20687. found, 537.20590.

### Example 107

### 5-((1-(2-(3-Fluoro-6-methoxy-1,5-naphthyridin-4-yl)ethyl)-2-oxabicyclo[2.2.2]octan-4-ylamino)methyl)oxazolo[4,5-b]pyridin-2(3H)-one

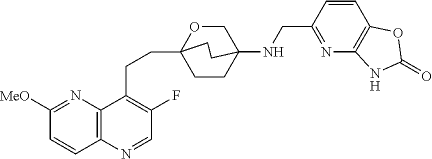 [SMILES: COc1ccc2ncc(F)c(CCC34CCC(NCc5ccc6oc(=O)[nH]c6n5)(CC3)CO4)c2n1]

The title compound was prepared from 2-oxo-2,3-dihydro[1,3]oxazolo[4,5-b]pyridine-5-carbaldehyde (AN).

¹H NMR (DMSO-d₆): δ 1.59-1.96 (m, 11H), 3.07-3.15 (m, 2H), 3.63 (s, 2H), 3.75 (s, 2H), 4.05 (s, 3H), 7.07 (d, J=8.0 Hz, 1H), 7.22 (d, J=9.2 Hz, 1H), 7.48 (d, J=8.6 Hz, 1H), 8.26 (d, J=9.2 Hz, 1H), 8.74 (s, 1H).

MS (ESI⁺) m/z: 480 (MH⁺).

HRMS (ESI⁺) for C₂₅H₂₇FN₅O₄ (MH⁺): calcd, 480.20471. found, 480.20535.

### Example 108

### 2-((1-(2-(3-Fluoro-6-methoxy-1,5-naphthyridin-4-yl)ethyl)-2-oxabicyclo[2.2.2]octan-4-ylamino)methyl)-4-methylpyrido[3,2-b]pyrazin-3(4H)-one Hydrochloride

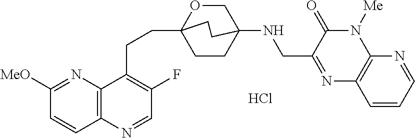 [SMILES: COc1ccc2ncc(F)c(CCC34CCC(NCc5nc6cccnc6n(C)c5=O)(CC3)CO4)c2n1 ; Cl]

The title compound was prepared from 4-methyl-3-oxo-3,4-dihydropyrido[2,3-b]pyrazine-2-carbaldehyde (AP).

¹H NMR (DMSO-d₆): δ 1.68-1.76 (m, 2H), 1.81-1.90 (m, 2H), 1.95-2.13 (m, 6H), 3.10-3.18 (m, 2H), 3.72 (s, 3H), 3.96 (s, 2H), 4.05 (s, 3H), 4.36 (s, 2H), 7.24 (d, J=9.2 Hz, 1H), 7.53 (dd, J=8.0, 4.9 Hz, 1H), 8.28 (d, J=9.2 Hz, 1H), 8.35 (dd, J=8.0, 1.8 Hz, 1H), 8.73 (dd, J=4.9, 1.8 Hz, 1H), 8.77 (s, 1H), 9.49 (s, 1H).

MS (ESI⁺) m/z: 505 (MH⁺) (as free base).

HRMS (ESI⁺) for C₂₇H₃₀FN₆O₃ (MH⁺) (as free base): calcd, 505.23634. found, 505.23651.

### Example 109

### 6-((1-(2-(3-Oxo-2H-pyrido[3,2-b][1,4]oxazin-4(3H)-yl)ethyl)-2-oxabicyclo[2.2.2]octan-4-ylamino)methyl)-2H-pyrido[3,2-b][1,4]oxazin-3(4H)-one Hydrochloride

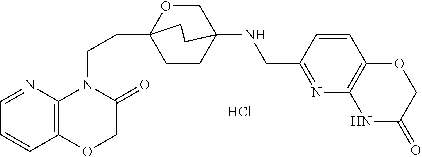 [SMILES: O=C1COc2ccc(CNC34CCC(CCN5C(=O)COc6cccnc65)(CC3)OC4)nc2N1 ; Cl]

### Step 1

### tert-Butyl 1-(2-(3-Oxo-2H-pyrido[3,2-b][1,4]oxazin-4(3H)-yl)ethyl)-2-oxabicyclo[2.2.2]octan-4-ylcarbamate

To a suspension of sodium hydride (38.0 mg, 55%) in N,N-dimethylacetamide (5 mL) was added 2H-pyrido[3,2-b][1,4]oxazin-3(4H)-one (151 mg) under cooling with ice, the mixture was stirred at room temperature for 30 minutes. The mixture was added AC (151 mg) under cooling with ice, the mixture was stirred at room temperature for 1.5 hours and further stirred at 60° C. for 4 hours. The mixture was concentrated in vacuo. After dilution of the residue with ethyl acetate, the mixture was added saturated ammonium chloride solution under cooling with ice. The organic extracts were washed with brine, dried over anhydrous sodium sulfate, filtered, and then concentrated in vacuo. Flash chromatography (silica, chloroform: ethyl acetate=2:1) of the residue gave tert-butyl 1-(2-(3-oxo-2H-pyrido[3,2-b][1,4]oxazin-4(3H)-yl)ethyl)-2-oxabicyclo[2.2.2]octan-4-ylcarbamate (84.0 mg).

¹H NMR (CDCl₃): δ 1.42 (s, 9H), 1.69-2.10 (m, 10H), 3.90 (s, 2H), 4.16-4.29 (m, 3H), 4.62 (d, J=3.7 Hz, 2H), 6.90 (dd, J=8.0, 4.9 Hz, 1H), 7.19 (dd, J=7.3, 1.2 Hz, 1H), 8.01 (dd, J=4.8, 1.2, Hz, 1H).

MS (ESI⁺) m/z: 404 (MH⁺).

HRMS (ESI⁺) for C₂₁H₃₀N₃O₅ (MH⁺): calcd, 404.21855. found, 404.21800.

### Step 2

### 4-(2-(4-Amino-2-oxabicyclo[2.2.2]octan-1-yl)ethyl)-2H-pyrido[3,2-b][1,4]oxazin-3(4H)-one

The title compound 4-(2-(4-amino-2-oxabicyclo[2.2.2]octan-1-yl)ethyl)-2H-pyrido[3,2-b][1,4]oxazin-3(4H)-one (49.5 mg) was prepared from tert-butyl 1-(2-(3-oxo-2H-pyrido[3,2-b][1,4]oxazin-4(3H)-yl)ethyl)-2-oxabicyclo[2.2.2]octan-4-ylcarbamate (66.0 mg) in the same manner as described for Step 2 of EXAMPLE 1.

¹H NMR (CDCl₃): δ 1.40 (brs, 2H), 1.56-1.82 (m, 8H), 1.94-2.08 (m, 2H), 3.60 (s, 2H), 4.17-4.26 (m, 2H), 4.63 (s, 2H), 6.90 (dd, J=7.9, 4.9 Hz, 1H), 7.19 (dd, J=7.9, 1.8 Hz, 1H), 8.02 (dd, J=4.9, 1.8 Hz, 1H).

MS (ESI⁺) m/z: 304 (MH⁺).

HRMS (ESI⁺) for C₁₆H₂₂N₃O₃ (MH⁺): calcd, 304.16612. found, 304.16603.

### Step 3

### 6-((1-(2-(3-Oxo-2H-pyrido[3,2-b][1,4]oxazin-4(3H)-yl)ethyl)-2-oxabicyclo[2.2.2]octan-4-ylamino)methyl)-2H-pyrido[3,2-b][1,4]oxazin-3(4H)-one

The title compound 6-((1-(2-(3-oxo-2H-pyrido[3,2-b][1,4]oxazin-4(3H)-yl)ethyl)-2-oxabicyclo[2.2.2]octan-4-ylamino)methyl)-2H-pyrido[3,2-b][1,4]oxazin-3(4H)-one (74.6 mg) was prepared from 4-(2-(4-amino-2-oxabicyclo[2.2.2]octan-1-yl)ethyl)-2H-pyrido[3,2-b][1,4]oxazin-3(4H)-one (60.0 mg) and I (37.0 mg) in the same manner as described for Step 3 of EXAMPLE 1.

¹H NMR (DMSO-d₆): δ 1.54-1.71 (m, 8H), 1.76-1.91 (m, 3H), 3.53 (s, 2H), 3.61 (d, J=6.7 Hz, 2H), 3.99-4.10 (m, 2H), 4.58 (s, 2H), 4.71 (s, 2H), 6.97-7.05 (m, 2H), 7.27 (d, J=7.9 Hz, 1H), 7.36 (dd, J=7.9, 1.2 Hz, 1H), 8.01 (dd, J=4.9, 1.2 Hz, 1H), 11.14 (s, 1H).

MS (ESI⁺) m/z: 466 (MH⁺).

HRMS (ESI⁺) for C₂₄H₂₈N₅O₅ (MH⁺): calcd, 466.20904. found, 466.20926.

### Step 4

### 6-((1-(2-(3-Oxo-2H-pyrido[3,2-b][1,4]oxazin-4(3H)-yl)ethyl)-2-oxabicyclo[2.2.2]octan-4-ylamino)methyl)-2H-pyrido[3,2-b][1,4]oxazin-3(4H)-one Hydrochloride

The title compound 6-((1-(2-(3-oxo-2H-pyrido[3,2-b][1,4]oxazin-4(3H)-yl)ethyl)-2-oxabicyclo[2.2.2]octan-4-ylamino)methyl)-2H-pyrido[3,2-b][1,4]oxazin-3(4H)-one hydrochloride (51.7 mg) was prepared from 6-((1-(2-(3-oxo-2H-pyrido[3,2-b][1,4]oxazin-4(3H)-yl)ethyl)-2-oxabicyclo[2.2.2]octan-4-ylamino)methyl)-2H-pyrido[3,2-b][1,4]oxazin-3(4H)-one (60.0 mg) in the same manner as described for Step 4 of EXAMPLE 3.

¹H NMR (DMSO-d₆): δ 1.58-1.70 (m, 2H), 1.72-1.84 (m, 2H), 1.88-2.05 (m, 6H), 3.86 (s, 2H), 4.02-4.14 (m, 4H), 4.68 (s, 2H), 4.72 (s, 2H), 7.04 (dd, J=7.9, 4.9 Hz, 1H), 7.18 (br, 1H), 7.37 (dd, J=7.9, 1.2 Hz, 1H), 7.45 (d, J=8.6 Hz, 1H), 8.01 (dd, J=4.9, 1.2 Hz, 1H), 9.22 (br, 2H), 11.32 (s, 1H).

MS (ESI⁺) m/z: 466 (MH⁺) (as free base).

HRMS (ESI⁺) for C₂₄H₂₈N₅O₅ (MH⁺) (as free base): calcd, 466.20904. found, 466.20846.

### Example 110

### 6-((1-(2-(5-Methyl-2-oxo-1,6-naphthyridin-1(2H)-yl)ethyl)-2-oxabicyclo[2.2.2]octan-4-ylamino)methyl)-2H-pyrido[3,2-b][1,4]oxazin-3(4H)-one

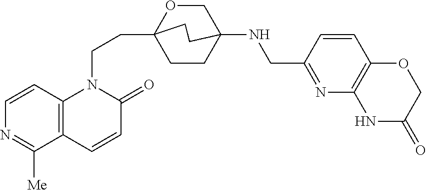 [SMILES: Cc1nccc2c1ccc(=O)n2CCC12CCC(NCc3ccc4c(n3)NC(=O)CO4)(CC1)CO2]

### Step 1

### tert-Butyl 1-(2-(5-Methyl-2-oxo-1,6-naphthyridin-1(2H)-yl)ethyl)-2-oxabicyclo[2.2.2]octan-4-ylcarbamate

The title compound tert-butyl 1-(2-(5-methyl-2-oxo-1,6-naphthyridin-1(2H)-yl)ethyl)-2-oxabicyclo[2.2.2]octan-4-ylcarbamate (60.0 mg) was prepared from 5-methyl-1,6-naphthyridin-2(1H)-one (275 mg) and AC (300 mg) in the same manner as described for Step 1 of EXAMPLE 109.

¹H NMR (CDCl₃): δ 1.44 (s, 9H), 1.68-1.79 (m, 4H), 1.82-1.91 (m, 2H), 1.95-2.17 (m, 4H), 2.78 (s, 3H), 4.01 (s, 2H), 4.26-4.37 (m, 3H), 6.71 (d, J=9.8 Hz, 1H), 7.27 (d, J=6.7 Hz, 1H), 7.92 (d, J=9.8 Hz, 1H), 8.48 (d, J=6.1 Hz, 1H).

MS (ESI⁺) m/z: 414 (MH⁺).

HRMS (ESI⁺) for C₂₃H₃₂N₃O₄ (MH⁺): calcd, 414.23928. found, 414.23862.

### Step 2

### 1-(2-(4-Amino-2-oxabicyclo[2.2.2]octan-1-yl)ethyl)-5-methyl-1,6-naphthyridin-2(1H)-one

The title compound 1-(2-(4-amino-2-oxabicyclo[2.2.2]octan-1-yl)ethyl)-5-methyl-1,6-naphthyridin-2(1H)-one (37.8 mg) was prepared from tert-butyl 1-(2-(5-methyl-2-oxo-1,6-naphthyridin-1(2H)-yl)ethyl)-2-oxabicyclo[2.2.2]octan-4-ylcarbamate (50.0 mg) in the same manner as described for Step 2 of EXAMPLE 1.

¹H NMR (CDCl₃): δ 1.44 (brs, 2H), 1.62-1.79 (m, 8H), 1.90-2.04 (m, 2H), 2.78 (s, 3H), 3.68 (s, 2H), 4.28-4.36 (m, 2H), 6.71 (d, J=9.8 Hz, 1H), 7.27 (d, J=8.6 Hz, 1H), 7.92 (d, J=9.8 Hz, 1H), 8.48 (d, J=6.1 Hz, 1H).

MS (ESI⁺) m/z: 314 (MH⁺).

HRMS (ESI⁺) for C₁₈H₂₄N₃O₂ (MH⁺): calcd, 314.18685. found, 314.18683.

### Step 3

### 6-((1-(2-(5-Methyl-2-oxo-1,6-naphthyridin-1(2H)-yl)ethyl)-2-oxabicyclo[2.2.2]octan-4-ylamino)methyl)-2H-pyrido[3,2-b][1,4]oxazin-3(4H)-one

The title compound 6-((1-(2-(5-methyl-2-oxo-1,6-naphthyridin-1(2H)-yl)ethyl)-2-oxabicyclo[2.2.2]octan-4-ylamino)methyl)-2H-pyrido[3,2-b][1,4]oxazin-3(4H)-one (28.0 mg) was prepared from 1-(2-(4-amino-2-oxabicyclo[2.2.2]octan-1-yl)ethyl)-5-methyl-1,6-naphthyridin-2(1H)-one (30.0 mg) and I (17.9 mg) in the same manner as described for Step 3 of EXAMPLE 1.

¹H NMR (DMSO-d₆): δ 1.52-1.76 (m, 8H), 1.79-1.96 (m, 3H), 2.70 (s, 3H), 3.62 (s, 2H), 3.64 (s, 2H), 4.16-4.23 (m, 2H), 4.58 (s, 2H), 6.65 (d, J=9.8 Hz, 1H), 7.01 (d, J=7.9 Hz, 1H), 7.21 (d, J=5.5 Hz, 1H), 7.28 (d, J=8.6 Hz, 1H), 8.12 (d, J=9.8 Hz, 1H), 8.44 (d, J=6.1 Hz, 1H), 11.15 (br, 1H).

MS (ESI⁺) m/z: 476 (MH⁺).

HRMS (ESI⁺) for C₂₆H₃₀N₅O₄ (MH⁺): calcd, 476.22978. found, 476.22963.

### Example 111

### 6-((1-(2-(7-Methyl-2-oxo-1,8-naphthyridin-1(2H)-yl)ethyl)-2-oxabicyclo[2.2.2]octan-4-ylamino)methyl)-2H-pyrido[3,2-b][1,4]oxazin-3(4H)-one Hydrochloride

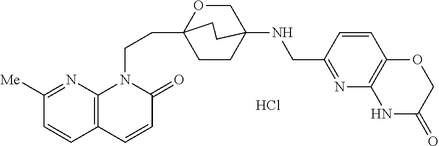 [SMILES: Cc1ccc2ccc(=O)n(CCC34CCC(NCc5ccc6c(n5)NC(=O)CO6)(CC3)CO4)c2n1 ; Cl]

### Step 1

### tert-Butyl 1-(2-(7-Methyl-2-oxo-1,8-naphthyridin-1(2H)-yl)ethyl)-2-oxabicyclo[2.2.2]octan-4-ylcarbamate and tert-Butyl 1-(2-(7-Methyl-1,8-naphthyridin-2-yloxy)ethyl)-2-oxabicyclo[2.2.2]octan-4-ylcarbamate

The title compound tert-butyl 1-(2-(7-methyl-2-oxo-1,8-naphthyridin-1(2H)-yl)ethyl)-2-oxabicyclo[2.2.2]octan-4-ylcarbamate (181 mg) and tert-butyl 1-(2-(7-methyl-1,8-naphthyridin-2-yloxy)ethyl)-2-oxabicyclo[2.2.2]octan-4-ylcarbamate (75.4 mg) was prepared from 7-methyl-1,8-naphthyridin-2(1H)-one (275 mg) and AC (300 mg) in the same manner as described for Step 1 of EXAMPLE 109.

tert-Butyl 1-(2-(7-Methyl-2-oxo-1,8-naphthyridin-1(2H)-yl)ethyl)-2-oxabicyclo[2.2.2]octan-4-ylcarbamate: ¹H NMR (CDCl₃): δ 1.43 (s, 9H), 1.75-1.95 (m, 6H), 2.01-2.15 (m, 4H), 2.62 (s, 3H), 3.93 (s, 2H), 4.26 (br, 1H), 4.41-4.60 (m, 2H), 6.65 (d, J=9.8 Hz, 1H), 7.00 (d, J=7.9 Hz, 1H), 7.56 (d, J=9.2 Hz, 1H), 7.70 (d, J=7.9 Hz, 1H).

MS (ESI⁺) m/z: 414 (MH⁺).

HRMS (ESI⁺) for C₂₃H₃₂N₃O₄ (MH⁺): calcd, 414.23928. found, 414.23975.

tert-Butyl 1-(2-(7-Methyl-1,8-naphthyridin-2-yloxy)ethyl)-2-oxabicyclo[2.2.2]octan-4-ylcarbamate: ¹H NMR (CDCl₃): δ 1.43 (s, 9H), 1.73-1.89 (m, 6H), 1.93-2.11 (m, 4H), 2.76 (s, 3H), 3.93 (s, 2H), 4.25 (br, 1H), 4.64 (t, J=6.7 Hz, 2H), 6.89 (d, J=8.6 Hz, 1H), 7.22 (d, J=8.0 Hz, 1H), 7.93 (d, J=9.2 Hz, 1H), 7.95 (d, J=8.0 Hz, 1H).

MS (ESI⁺) m/z: 414 (MH⁺).

HRMS (ESI⁺) for C₂₃H₃₂N₃O₄ (MH⁺): calcd, 414.23928. found, 414.23911.

### Step 2

### 1-(2-(4-Amino-2-oxabicyclo[2.2.2]octan-1-yl)ethyl)-7-methyl-1,8-naphthyridin-2(1H)-one

The title compound 1-(2-(4-amino-2-oxabicyclo[2.2.2]octan-1-yl)ethyl)-7-methyl-1,8-naphthyridin-2(1H)-one (152 mg) was prepared from tert-butyl 1-(2-(7-methyl-2-oxo-1,8-naphthyridin-1(2H)-yl)ethyl)-2-oxabicyclo[2.2.2]octan-4-ylcarbamate (164 mg) in the same manner as described for Step 2 of EXAMPLE 1.

¹H NMR (DMSO-d₆): δ 1.29 (s, 2H), 1.46-1.72 (m, 8H), 1.78-1.92 (m, 2H), 2.56 (s, 3H), 3.42 (s, 2H), 4.34-4.43 (m, 2H), 6.57 (d, J=9.2 Hz, 1H), 7.16 (d, J=7.3 Hz, 1H), 7.86 (d, J=9.2 Hz, 1H), 8.02 (d, J=7.9 Hz, 1H).

MS (ESI⁺) m/z: 314 (MH⁺).

HRMS (ESI⁺) for C₁₈H₂₄N₃O₂ (MH⁺): calcd, 314.18685. found, 314.18681.

### Step 3

### 6-((1-(2-(7-Methyl-2-oxo-1,8-naphthyridin-1(2H)-yl)ethyl)-2-oxabicyclo[2.2.2]octan-4-ylamino)methyl)-2H-pyrido[3,2-b][1,4]oxazin-3(4H)-one

The title compound 6-((1-(2-(7-methyl-2-oxo-1,8-naphthyridin-1(2H)-yl)ethyl)-2-oxabicyclo[2.2.2]octan-4-ylamino)methyl)-2H-pyrido[3,2-b][1,4]oxazin-3(4H)-one (116 mg) was prepared from 1-(2-(4-amino-2-oxabicyclo[2.2.2]octan-1-yl)ethyl)-7-methyl-1,8-naphthyridin-2(1H)-one (90.0 mg) and I (53.7 mg) in the same manner as described for Step 3 of EXAMPLE 1.

¹H NMR (DMSO-d₆): δ 1.54-1.77 (m, 8H), 1.82-1.97 (m, 3H), 2.56 (s, 3H), 3.55 (s, 2H), 3.62 (s, 2H), 4.35-4.45 (m, 2H), 4.59 (s, 2H), 6.57 (d, J=9.2 Hz, 1H), 7.01 (d, J=7.9 Hz, 1H), 7.16 (d, J=7.9 Hz, 1H), 7.27 (d, J=7.9 Hz, 1H), 7.86 (d, J=9.8 Hz, 1H), 8.02 (d, J=7.9 Hz, 1H), 11.15 (br, 1H).

MS (ESI⁺) m/z: 476 (MH⁺).

HRMS (ESI⁺) for C₂₆H₃₀N₅O₄ (MH⁺): calcd, 476.22978. found, 476.22887.

### Step 4

### 6-((1-(2-(7-Methyl-2-oxo-1,8-naphthyridin-1(2H)-yl)ethyl)-2-oxabicyclo[2.2.2]octan-4-ylamino)methyl)-2H-pyrido[3,2-b][1,4]oxazin-3(4H)-one Hydrochloride

The title compound 6-((1-(2-(7-methyl-2-oxo-1,8-naphthyridin-1(2H)-yl)ethyl)-2-oxabicyclo[2.2.2]octan-4-ylamino)methyl)-2H-pyrido[3,2-b][1,4]oxazin-3(4H)-one hydrochloride (96.5 mg) was prepared from 6-((1-(2-(7-methyl-2-oxo-1,8-naphthyridin-1(2H)-yl)ethyl)-2-oxabicyclo[2.2.2]octan-4-ylamino)methyl)-2H-pyrido[3,2-b][1,4]oxazin-3(4H)-one (100 mg) in the same manner as described for Step 4 of EXAMPLE 3.

¹H NMR (DMSO-d₆): δ 1.65-1.69 (m, 2H), 1.77-1.90 (m, 2H), 2.01 (s, 6H), 2.57 (s, 3H), 3.88 (s, 2H), 4.10 (s, 2H), 4.39-4.44 (m, 2H), 4.69 (s, 2H), 6.59 (d, J=9.2 Hz, 1H), 7.18 (d, J=7.9 Hz, 1H), 7.20 (d, J=9.2 Hz, 1H), 7.45 (d, J=7.9 Hz, 1H), 7.88 (d, J=9.8 Hz, 1H), 8.04 (d, J=7.9 Hz, 1H), 9.24 (s, 2H), 11.32 (s, 1H).

MS (ESI⁺) m/z: 476 (MH⁺) (as free base).

HRMS (ESI⁺) for C₂₆H₃₀N₅O₄ (MH⁺) (as free base): calcd, 476.22978. found, 476.23024.

### Example 112

### 6-((1-(2-(9-Fluoro-4-oxopyrrolo[1,2-a]quinoxalin-5(4H)-yl)ethyl)-2-oxabicyclo[2.2.2]octan-4-ylamino)methyl)-2H-pyrido[3,2-b][1,4]oxazin-3(4H)-one

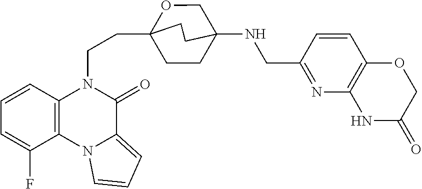 [SMILES: O=C1COc2ccc(CNC34CCC(CCn5c(=O)c6cccn6c6c(F)cccc65)(CC3)OC4)nc2N1]

### Step 1

### tert-Butyl 1-(2-(9-Fluoro-4-oxopyrrolo[1,2-a]quinoxalin-5(4H)-yl)ethyl)-2-oxabicyclo[2.2.2]octan-4-ylcarbamate

The title compound tert-butyl 1-(2-(9-fluoro-4-oxopyrrolo[1,2-a]quinoxalin-5(4H)-yl)ethyl)-2-oxabicyclo[2.2.2]octan-4-ylcarbamate (67.5 mg) was prepared from 9-fluoropyrrolo[1,2-a]quinoxalin-4(5H)-one (202 mg) and AC (175 mg) in the same manner as described for Step 1 of EXAMPLE 109.

¹H NMR (CDCl₃): δ 1.44 (s, 9H), 1.73-1.89 (m, 6H), 2.00-2.17 (m, 4H), 4.01 (s, 2H), 4.25-4.37 (m, 3H), 6.66 (dd, J=4.3, 3.1 Hz, 1H), 6.99-7.07 (m, 1H), 7.23-7.30 (m, 3H), 7.99-8.03 (m, 1H).

MS (ESI⁺) m/z: 456 (MH⁺).

HRMS (ESI⁺) for C₂₅H₃₁FN₃O₄ (MH⁺): calcd, 456.22986. found, 456.22922.

### Step 2

### 5-(2-(4-Amino-2-oxabicyclo[2.2.2]octan-1-yl)ethyl)-9-fluoropyrrolo[1,2-a]quinoxalin-4(5H)-one

The title compound 5-(2-(4-amino-2-oxabicyclo[2.2.2]octan-1-yl)ethyl)-9-fluoropyrrolo[1,2-a]quinoxalin-4(5H)-one (47.1 mg) was prepared from tert-butyl 1-(2-(9-fluoro-4-oxopyrrolo[1,2-a]quinoxalin-5(4H)-yl)ethyl)-2-oxabicyclo[2.2.2]octan-4-ylcarbamate (63.4 mg) in the same manner as described for Step 2 of EXAMPLE 1.

¹H NMR (CDCl₃): δ 1.21-1.62 (m, 2H), 1.63-1.81 (m, 8H), 1.97-2.03 (m, 2H), 3.68 (s, 2H), 4.32-4.34 (m, 2H), 6.66 (dd, J=4.3, 3.1 Hz, 1H), 7.00-7.06 (m, 1H), 7.23-7.30 (m, 3H), 8.01-8.02 (m, 1H).

MS (ESI⁺) m/z: 356 (MH⁺).

HRMS (ESI⁺) for C₂₀H₂₃FN₃O₂ (MH⁺): calcd, 356.17743. found, 356.17712.

### Step 3

### 6-((1-(2-(9-Fluoro-4-oxopyrrolo[1,2-a]quinoxalin-5(4H)-yl)ethyl)-2-oxabicyclo[2.2.2]octan-4-ylamino)methyl)-2H-pyrido[3,2-b][1,4]oxazin-3(4H)-one

The title compound 6-((1-(2-(9-fluoro-4-oxopyrrolo[1,2-a]quinoxalin-5(4H)-yl)ethyl)-2-oxabicyclo[2.2.2]octan-4-ylamino)methyl)-2H-pyrido[3,2-b][1,4]oxazin-3(4H)-one (18.0 mg) was prepared from 5-(2-(4-amino-2-oxabicyclo[2.2.2]octan-1-yl)ethyl)-9-fluoropyrrolo[1,2-a]quinoxalin-4(5H)-one (44.0 mg) and I (23.0 mg) in the same manner as described for Step 3 of EXAMPLE 1.

¹H NMR (CDCl₃): δ 1.57 (m, 1H), 1.70-1.89 (m, 8H), 1.94-2.10 (m, 2H), 3.76 (s, 2H), 3.80 (s, 2H), 4.33-4.37 (m, 2H), 4.63 (s, 2H), 6.66 (t, J=3.7 Hz, 1H), 6.95 (d, J=8.6 Hz, 1H), 6.99-7.07 (m, 1H), 7.20 (d, J=7.9 Hz, 1H), 7.22-7.30 (m, 3H), 7.99-8.03 (m, 1H), 8.22 (br, 1H).

MS (ESI⁺) m/z: 518 (MH⁺).

HRMS (ESI⁺) for C₂₈H₂₉FN₅O₄ (MH⁺): calcd, 518.22036. found, 518.21968.

### Example 113

### 6-((1-(2-(7-Methyl-1,8-naphthyridin-2-yloxy)ethyl)-2-oxabicyclo[2.2.2]octan-4-ylamino)methyl)-2H-pyrido[3,2-b][1,4]oxazin-3(4H)-one

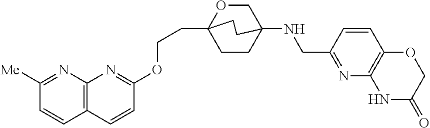 [SMILES: Cc1ccc2ccc(OCCC34CCC(NCc5ccc6c(n5)NC(=O)CO6)(CC3)CO4)nc2n1]

### Step 1

### 1-(2-(7-Methyl-1,8-naphthyridin-2-yloxy)ethyl)-2-oxabicyclo[2.2.2]octan-4-amine

The title compound 1-(2-(7-methyl-1,8-naphthyridin-2-yloxy)ethyl)-2-oxabicyclo[2.2.2]octan-4-amine (48.1 mg) was prepared from tert-butyl 1-(2-(7-methyl-1,8-naphthyridin-2-yloxy)ethyl)-2-oxabicyclo[2.2.2]octan-4-ylcarbamate (68.0 mg) in the same manner as described for Step 2 of EXAMPLE 1.

¹H NMR (CDCl₃): δ 1.60-1.84 (m, 8H), 1.91-2.03 (m, 4H), 2.76 (s, 3H), 3.62 (s, 2H), 4.64 (t, J=7.3 Hz, 2H), 6.90 (d, J=9.2 Hz, 1H), 7.22 (d, J=7.9 Hz, 1H), 7.93 (d, J=8.6 Hz, 1H), 7.95 (d, J=8.6 Hz, 1H).

MS (ESI⁺) m/z: 314 (MH⁺).

HRMS (ESI⁺) for C₁₈H₂₄N₃O₂ (MH⁺): calcd, 314.18685. found, 314.18596.

### Step 2

### 6-((1-(2-(7-Methyl-1,8-naphthyridin-2-yloxy)ethyl)-2-oxabicyclo[2.2.2]octan-4-ylamino)methyl)-2H-pyrido[3,2-b][1,4]oxazin-3(4H)-one

The title compound 6-((1-(2-(7-methyl-1,8-naphthyridin-2-yloxy)ethyl)-2-oxabicyclo[2.2.2]octan-4-ylamino)methyl)-2H-pyrido[3,2-b][1,4]oxazin-3(4H)-one (34.5 mg) was prepared from 1-(2-(7-methyl-1,8-naphthyridin-2-yloxy)ethyl)-2-oxabicyclo[2.2.2]octan-4-amine (30.0 mg) and I (17.9 mg) in the same manner as described for Step 3 of EXAMPLE 1.

¹H NMR (DMSO-d₆): δ 1.56-1.78 (m, 6H), 1.79-1.93 (m, 5H), 2.62 (s, 3H), 3.59 (s, 2H), 3.62 (s, 2H), 4.46 (t, J=7.3 Hz, 2H), 4.58 (s, 2H), 6.98 (d, J=8.6 Hz, 1H), 7.00 (d, J=6.1 Hz, 1H), 7.27 (d, J=7.9 Hz, 1H), 7.34 (d, J=8.6 Hz, 1H), 8.20 (d, J=7.9 Hz, 1H), 8.22 (d, J=8.6 Hz, 1H), 11.14 (s, 1H).

MS (ESI⁺) m/z: 476 (MH⁺).

HRMS (ESI⁺) for C₂₆H₃₀N₅O₄ (MH⁺): calcd, 476.22978. found, 476.22944.

### Example 114

### 6-((1-(2-(3-Methyl-2-oxoquinoxalin-1(2H)-yl)ethyl)-2-oxabicyclo[2.2.2]octan-4-ylamino)methyl)-2H-pyrido[3,2-b][1,4]oxazin-3(4H)-one

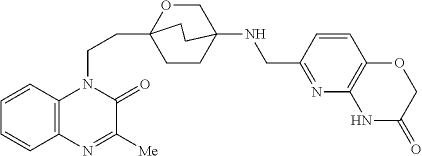 [SMILES: Cc1nc2ccccc2n(CCC23CCC(NCc4ccc5c(n4)NC(=O)CO5)(CC2)CO3)c1=O]

### Step 1

### tert-Butyl 1-(2-(3-Methyl-2-oxoquinoxalin-1(2H)-yl)ethyl)-2-oxabicyclo[2.2.2]octan-4-ylcarbamate

The title compound tert-butyl 1-(2-(3-methyl-2-oxoquinoxalin-1(2H)-yl)ethyl)-2-oxabicyclo[2.2.2]octan-4-ylcarbamate (33.8 mg) was prepared from 3-methylquinoxalin-2(1H)-one (240 mg) and AC (262 mg) in the same manner as described for Step 1 of EXAMPLE 109.

¹H NMR (CDCl₃): δ 1.44 (s, 9H), 1.70-1.90 (m, 6H), 1.93-2.21 (m, 4H), 2.58 (s, 3H), 4.01 (s, 2H), 4.24-4.39 (m, 3H), 7.32 (t, J=7.3 Hz, 1H), 7.45 (d, J=7.9 Hz, 1H), 7.53 (t, J=8.6 Hz, 1H), 7.80 (d, J=7.9 Hz, 1H).

MS (ESI⁺) m/z: 414 (MH⁺).

HRMS (ESI⁺) for C₂₃H₃₂N₃O₄ (MH⁺): calcd, 414.23928. found, 414.23971.

### Step 2

### 1-(2-(4-Amino-2-oxabicyclo[2.2.2]octan-1-yl)ethyl)-3-methylquinoxalin-2(1H)-one

The title compound 1-(2-(4-amino-2-oxabicyclo[2.2.2]octan-1-yl)ethyl)-3-methylquinoxalin-2(1H)-one (29.4 mg) was prepared from tert-butyl 1-(2-(3-methyl-2-oxoquinoxalin-1(2H)-yl)ethyl)-2-oxabicyclo[2.2.2]octan-4-ylcarbamate (39.4 mg) in the same manner as described for Step 2 of EXAMPLE 1.

¹H NMR (CDCl₃): δ 1.65-1.81 (m, 8H), 1.98-2.17 (m, 2H), 2.59 (s, 3H), 3.68 (s, 2H), 4.33-4.37 (m, 2H), 7.30-7.34 (m, 1H), 7.45 (d, J=7.3 Hz, 1H), 7.51-7.75 (m, 1H), 7.80 (dd, J=7.9, 1.2 Hz, 1H).

MS (ESI⁺) m/z: 314 (MH⁺).

HRMS (ESI⁺) for C₁₈H₂₃FN₃O₂ (MH⁺): calcd, 314.18685. found, 314.18634.

### Step 3

### 6-((1-(2-(3-Methyl-2-oxoquinoxalin-1(2H)-yl)ethyl)-2-oxabicyclo[2.2.2]octan-4-ylamino)methyl)-2H-pyrido[3,2-b][1,4]oxazin-3(4H)-one

The title compound 6-((1-(2-(3-methyl-2-oxoquinoxalin-1(2H)-yl)ethyl)-2-oxabicyclo[2.2.2]octan-4-ylamino)methyl)-2H-pyrido[3,2-b][1,4]oxazin-3(4H)-one (24.0 mg) was prepared from 1-(2-(4-amino-2-oxabicyclo[2.2.2]octan-1-yl)ethyl)-3-methylquinoxalin-2(1H)-one (29.0 mg) and I (17.3 mg) in the same manner as described for Step 3 of EXAMPLE 1.

¹H NMR (CDCl₃): δ 1.70-1.82 (m, 8H), 1.90-2.09 (m, 2H), 2.58 (s, 3H), 3.75 (s, 2H), 3.80 (s, 2H), 4.33-4.37 (m, 2H), 4.64 (s, 2H), 6.95 (d, J=7.9 Hz, 1H), 7.32 (t, J=7.9 Hz, 1H), 7.45 (d, J=8.6 Hz, 1H), 7.53 (t, J=8.6 Hz, 1H), 7.80 (d, J=7.9 Hz, 1H), 7.96 (br, 1H).

MS (ESI⁺) m/z: 476 (MH⁺).

HRMS (ESI⁺) for C₂₆H₃₀FN₅O₄ (MH⁺): calcd, 476.22978. found, 476.23046.

### Example 115

### 6-((1-(2-(6,7-Dimethoxy-2-oxoquinoxalin-1(2H)-yl)ethyl)-2-oxabicyclo[2.2.2]octan-4-ylamino)methyl)-2H-pyrido[3,2-b][1,4]oxazin-3(4H)-one

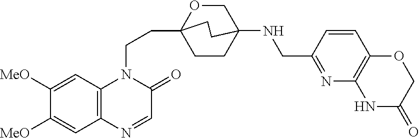 [SMILES: COc1cc2ncc(=O)n(CCC34CCC(NCc5ccc6c(n5)NC(=O)CO6)(CC3)CO4)c2cc1OC]

### Step 1

### tert-Butyl 1-(2-(6,7-Dimethoxy-2-oxoquinoxalin-1(2H)-yl)ethyl)-2-oxabicyclo[2.2.2]octan-4-ylcarbamate

The title compound tert-butyl 1-(2-(6,7-dimethoxy-2-oxoquinoxalin-1(2H)-yl)ethyl)-2-oxabicyclo[2.2.2]octan-4-ylcarbamate (30.0 mg) was prepared from 6,7-dimethoxyquinoxalin-2(1H)-one (300 mg) and AC (254 mg) in the same manner as described for Step 1 of EXAMPLE 109.

¹H NMR (CDCl₃): δ 1.43 (s, 9H), 1.67-1.86 (m, 6H), 1.94-2.05 (m, 2H), 2.11-2.23 (m, 2H), 3.95 (s, 3H), 4.02 (s, 3H), 4.05 (s, 2H), 4.32-4.37 (m, 3H), 7.22 (s, 1H), 7.28 (s, 1H), 8.13 (s, 1H).

MS (ESI⁺) m/z: 460 (MH⁺).

HRMS (ESI⁺) for C₂₄H₃₄N₃O₆ (MH⁺): calcd, 460.24476. found, 460.24448.

### Step 2

### 1-(2-(4-Amino-2-oxabicyclo[2.2.2]octan-1-yl)ethyl)-6,7-dimethoxyquinoxalin-2(1H)-one

The title compound 1-(2-(4-amino-2-oxabicyclo[2.2.2]octan-1-yl)ethyl)-6,7-dimethoxyquinoxalin-2(1H)-one (32.1 mg) was prepared from tert-butyl 1-(2-(6,7-dimethoxy-2-oxoquinoxalin-1(2H)-yl)ethyl)-2-oxabicyclo[2.2.2]octan-4-ylcarbamate (41.0 mg) in the same manner as described for Step 2 of EXAMPLE 1.

¹H NMR (CDCl₃): δ 1.65-1.83 (m, 8H), 1.89-2.00 (m, 2H), 3.66 (s, 2H), 3.96 (s, 3H), 4.02 (s, 3H), 4.31-4.39 (m, 2H), 7.23 (s, 1H), 7.28 (s, 1H), 8.14 (s, 1H).

### Step 3

### 6-((1-(2-(6,7-Dimethoxy-2-oxoquinoxalin-1(2H)-yl)ethyl)-2-oxabicyclo[2.2.2]octan-4-ylamino)methyl)-2H-pyrido[3,2-b][1,4]oxazin-3(4H)-one

The title compound 6-((1-(2-(6,7-dimethoxy-2-oxoquinoxalin-1(2H)-yl)ethyl)-2-oxabicyclo[2.2.2]octan-4-ylamino)methyl)-2H-pyrido[3,2-b][1,4]oxazin-3(4H)-one (27.0 mg) was prepared from 1-(2-(4-amino-2-oxabicyclo[2.2.2]octan-1-yl)ethyl)-6,7-dimethoxyquinoxalin-2(1H)-one (31.0 mg) and I (16.1 mg) in the same manner as described for Step 3 of EXAMPLE 1.

¹H NMR (CDCl₃): δ 1.72-1.84 (m, 9H), 1.95-2.05 (m, 2H), 3.80 (s, 2H), 3.75 (s, 2H), 3.96 (s, 3H), 4.02 (s, 3H), 4.34-4.38 (m, 2H), 4.64 (s, 2H), 6.94 (d, J=7.9 Hz, 1H), 7.21 (d, J=7.3 Hz, 1H), 7.23 (s, 1H), 7.29 (s, 1H), 8.06 (br, 1H), 8.14 (s, 1H).

MS (ESI⁺) m/z: 522 (MH⁺).

HRMS (ESI⁺) for C₂₇H₃₂N₅O₆ (MH⁺): calcd, 522.23526. found, 522.23585.

### Example 116

### 6-((1-(2-(2-Oxo-1,8-naphthyridin-1(2H)-yl)ethyl)-2-oxabicyclo[2.2.2]octan-4-ylamino)methyl)-2H-pyrido[3,2-b][1,4]oxazin-3(4H)-one

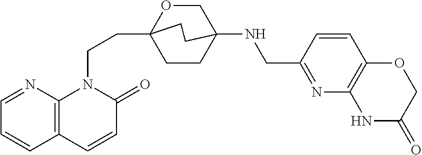 [SMILES: O=C1COc2ccc(CNC34CCC(CCn5c(=O)ccc6cccnc65)(CC3)OC4)nc2N1]

### Step 1

### tert-Butyl 1-(2-(2-Oxo-1,8-naphthyridin-1(2H)-yl)ethyl)-2-oxabicyclo[2.2.2]octan-4-ylcarbamate

The title compound tert-butyl 1-(2-(2-oxo-1,8-naphthyridin-1(2H)-yl)ethyl)-2-oxabicyclo[2.2.2]octan-4-ylcarbamate (70.0 mg) was prepared from 1,8-naphthyridin-2(1H)-one (300 mg) and AC (359 mg) in the same manner as described for Step 1 of EXAMPLE 109.

¹H NMR (CDCl₃): δ 1.43 (s, 9H), 1.73-1.87 (m, 4H), 1.95-2.10 (m, 6H), 3.93 (s, 2H), 4.26 (brs, 1H), 4.63-4.67 (m, 2H), 6.97 (d, J=9.2 Hz, 1H), 7.33 (dd, J=7.9, 4.9 Hz, 1H), 7.98 (d, J=8.6 Hz, 1H), 8.07 (dd, J=7.9, 2.4 Hz, 1H), 8.94 (dd, J=4.9, 2.4 Hz, 1H).

### Step 2

### 1-(2-(4-Amino-2-oxabicyclo[2.2.2]octan-1-yl)ethyl)-1,8-naphthyridin-2(1H)-one

The title compound 1-(2-(4-amino-2-oxabicyclo[2.2.2]octan-1-yl)ethyl)-1,8-naphthyridin-2(1H)-one (47.0 mg) was prepared from tert-butyl 1-(2-(2-oxo-1,8-naphthyridin-1(2H)-yl)ethyl)-2-oxabicyclo[2.2.2]octan-4-ylcarbamate (63.5 mg) in the same manner as described for Step 2 of EXAMPLE 1.

¹H NMR (CDCl₃): δ 1.60-1.82 (m, 6H), 1.95-2.07 (m, 4H), 3.63 (s, 2H), 4.66 (t, J=7.3 Hz, 2H), 6.97 (d, J=8.6 Hz, 1H), 7.33 (dd, J=7.9, 4.3 Hz, 1H), 7.99 (d, J=8.6 Hz, 1H), 8.08 (d, J=7.9 Hz, 1H), 8.94 (dd, J=4.9, 1.8 Hz, 1H).

### Step 3

### 6-((1-(2-(2-Oxo-1,8-naphthyridin-1(2H)-yl)ethyl)-2-oxabicyclo[2.2.2]octan-4-ylamino)methyl)-2H-pyrido[3,2-b][1,4]oxazin-3(4H)-one

The title compound 6-((1-(2-(2-oxo-1,8-naphthyridin-1(2H)-yl)ethyl)-2-oxabicyclo[2.2.2]octan-4-ylamino)methyl)-2H-pyrido[3,2-b][1,4]oxazin-3(4H)-one (30.0 mg) was prepared from 1-(2-(4-amino-2-oxabicyclo[2.2.2]octan-1-yl)ethyl)-1,8-naphthyridin-2(1H)-one (47.0 mg) and I (29.4 mg) in the same manner as described for Step 3 of EXAMPLE 1.

¹H NMR (CDCl₃): δ 1.55-1.65 (m, 2H), 1.76-1.84 (m, 6H), 1.97-2.04 (m, 4H), 3.74 (s, 2H), 3.75 (s, 2H), 4.63 (s, 2H), 4.67 (t, J=7.3 Hz, 2H), 6.93 (d, J=8.6 Hz, 1H), 6.97 (d, J=8.6 Hz, 1H), 7.19 (d, J=7.9 Hz, 1H), 7.34 (dd, J=7.9, 4.9 Hz, 1H), 7.99 (d, J=8.6 Hz, 1H), 8.08 (dd, J=7.9, 1.8 Hz, 1H), 8.94 (dd, J=4.9, 1.8 Hz, 1H).

MS (ESI⁺) m/z: 462 (MH⁺).

HRMS (ESI⁺) for C₂₅H₂₈N₅O₄ (MH⁺): calcd, 462.21413. found, 462.21483.

### Example 117

### 6-((1-(2-(1-Methyl-4-oxo-[1,2,4]triazolo[4,3-a]quinoxalin-5(4H)-yl)ethyl)-2-oxabicyclo[2.2.2]octan-4-ylamino)methyl)-2H-pyrido[3,2-b][1,4]oxazin-3(4H)-one

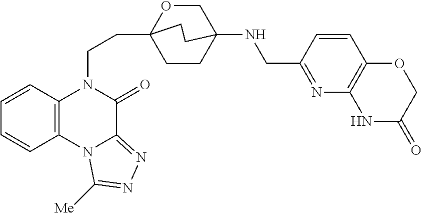 [SMILES: Cc1nnc2c(=O)n(CCC34CCC(NCc5ccc6c(n5)NC(=O)CO6)(CC3)CO4)c3ccccc3n12]

### Step 1

### tert-Butyl 1-(2-(1-Methyl-4-oxo-[1,2,4]triazolo[4,3-a]quinoxalin-5(4H)-yl)ethyl)-2-oxabicyclo[2.2.2]octan-4-ylcarbamate

The title compound tert-butyl 1-(2-(1-methyl-4-oxo-[1,2,4]triazolo[4,3-a]quinoxalin-5(4H)-yl)ethyl)-2-oxabicyclo[2.2.2]octan-4-ylcarbamate (88.8 mg) was prepared from 1-methyl-[1,2,4]triazolo[4,3-a]quinoxalin-4(5H)-one (200 mg) and AC (349 mg) in the same manner as described for Step 1 of EXAMPLE 109.

¹H NMR (CDCl₃): δ 1.45 (s, 9H), 1.72-1.91 (m, 6H), 2.00-2.18 (m, 4H), 3.09 (s, 3H), 4.04 (s, 2H), 4.31 (brs, 1H), 4.41-4.45 (m, 2H), 7.35-7.39 (m, 1H), 7.56 (dt, J=7.9, 1.2 Hz, 1H), 7.68 (d, J=8.6 Hz, 1H), 8.00 (dd, J=8.6, 1.2 Hz, 1H).

MS (ESI⁺) m/z: 454 (MH⁺).

HRMS (ESI⁺) for C₂₄H₃₂N₅O₄ (MH⁺): calcd, 454.24543. found, 454.24497.

### Step 2

### 5-(2-(4-Amino-2-oxabicyclo[2.2.2]octan-1-yl)ethyl)-1-methyl-[1,2,4]triazolo[4,3-a]quinoxalin-4(5H)-one

The title compound 5-(2-(4-amino-2-oxabicyclo[2.2.2]octan-1-yl)ethyl)-1-methyl-[1,2,4]triazolo[4,3-a]quinoxalin-4(5H)-one (57.3 mg) was prepared from tert-butyl 1-(2-(1-methyl-4-oxo-[1,2,4]triazolo[4,3-a]quinoxalin-5(4H)-yl)ethyl)-2-oxabicyclo[2.2.2]octan-4-ylcarbamate (80.0 mg) in the same manner as described for Step 2 of EXAMPLE 1.

¹H NMR (CDCl₃): δ 1.51-1.61 (m, 2H), 1.65-1.84 (m, 8H), 1.96-2.08 (m, 2H), 3.08 (s, 3H), 3.68 (s, 2H), 4.41-4.45 (m, 2H), 7.34-7.38 (m, 1H), 7.55 (dt, J=7.3, 1.2 Hz, 1H), 7.67 (d, J=8.6 Hz, 1H), 8.00 (dd, J=8.6, 1.2 Hz, 1H).

MS (ESI⁺) m/z: 354 (MH⁺).

HRMS (ESI⁺) for C₁₅H₂₄N₅O₂ (MH⁺): calcd, 354.19300. found, 354.19243.

### Step 3

### 6-((1-(2-(1-Methyl-4-oxo-[1,2,4]triazolo[4,3-a]quinoxalin-5(4H)-yl)ethyl)-2-oxabicyclo[2.2.2]octan-4-ylamino)methyl)-2H-pyrido[3,2-b][1,4]oxazin-3(4H)-one

The title compound 6-((1-(2-(1-methyl-4-oxo-[1,2,4]triazolo[4,3-a]quinoxalin-5(4H)-yl)ethyl)-2-oxabicyclo[2.2.2]octan-4-ylamino)methyl)-2H-pyrido[3,2-b][1,4]oxazin-3(4H)-one (43.5 mg) was prepared from 5-(2-(4-amino-2-oxabicyclo[2.2.2]octan-1-yl)ethyl)-1-methyl-[1,2,4]triazolo[4,3-a]quinoxalin-4(5H)-one (52.5 mg) and I (27.8 mg) in the same manner as described for Step 3 of EXAMPLE 1.

¹H NMR (CDCl₃): δ 1.75-1.88 (m, 9H), 2.00-2.05 (m, 2H), 3.08 (s, 3H), 3.76 (s, 2H), 3.80 (s, 2H), 4.41-4.46 (m, 2H), 4.63 (s, 2H), 6.95 (d, J=8.6 Hz, 1H), 7.21 (d, J=7.9 Hz, 1H), 7.36 (t, J=7.9 Hz, 1H), 7.56 (t, J=7.9 Hz, 1H), 7.66 (d, J=8.6 Hz, 1H), 8.00 (d, J=8.6 Hz, 1H), 8.13 (br, 1H).

MS (ESI⁺) m/z: 516 (MH⁺).

HRMS (ESI⁺) for C₂₇H₃₀N₇O₄ (MH⁺): calcd, 516.23593. found, 516.23633.

### Example 118

### 6-(((1-(2-(6-Oxo-3,4-dihydro-2H-pyrimido[1,2-c]quinazolin-7(6H)-yl)ethyl)-2-oxabicyclo[2.2.2]octan-4-yl)amino)methyl)-2H-pyrido[3,2-b][1,4]oxazin-3(4H)-one

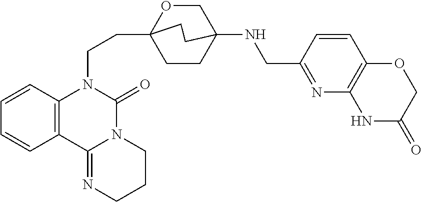 [SMILES: O=C1COc2ccc(CNC34CCC(CCN5C(=O)N6CCCN=C6c6ccccc65)(CC3)OC4)nc2N1]

### Step 1

### tert-Butyl 1-(2-(6-Oxo-3,4-dihydro-2H-pyrimido[1,2-c]quinazolin-7(6H)-yl)ethyl)-2-oxabicyclo[2.2.2]octan-4-ylcarbamate

The title compound tert-butyl 1-(2-(6-oxo-3,4-dihydro-2H-pyrimido[1,2-c]quinazolin-7(6H)-yl)ethyl)-2-oxabicyclo[2.2.2]octan-4-ylcarbamate (132 mg) was prepared from 3,4-dihydro-2H-pyrimido[1,2-c]quinazolin-6(7H)-one (84.0 mg) and AC (146 mg) in the same manner as described for Step 1 of EXAMPLE 109.

¹H NMR (CDCl₃): δ 1.43 (s, 9H), 1.67-2.05 (m, 12H), 3.62 (t, J=5.5 Hz, 2H), 3.90 (t, J=6.1 Hz, 2H), 3.97 (s, 2H), 4.08-4.12 (m, 2H), 4.28 (br, 1H), 7.10 (d, J=7.9 Hz, 1H), 7.16 (d, J=8.6 Hz, 1H), 7.45-7.49 (m, 1H), 8.15 (d, J=7.9 Hz, 1H).

MS (ESI⁺) m/z: 455 (MH⁺).

HRMS (ESI⁺) for C₂₅H₃₅N₄O₄ (MH⁺): calcd, 455.26583. found, 455.26676.

### Step 2

### 7-(2-(4-Amino-2-oxabicyclo[2.2.2]octan-1-yl)ethyl)-3,4-dihydro-2H-pyrimido[1,2-c]quinazolin-6(7H)-one

The title compound 7-(2-(4-amino-2-oxabicyclo[2.2.2]octan-1-yl)ethyl)-3,4-dihydro-2H-pyrimido[1,2-c]quinazolin-6(7H)-one (60.0 mg) was prepared from tert-butyl 1-(2-(6-oxo-3,4-dihydro-2H-pyrimido[1,2-c]quinazolin-7(6H)-yl)ethyl)-2-oxabicyclo[2.2.2]octan-4-ylcarbamate (100 mg) in the same manner as described for Step 2 of EXAMPLE 1.

¹H NMR (CDCl₃): δ 1.63-1.77 (m, 10H), 1.89-2.00 (m, 4H), 3.62 (t, J=6.1 Hz, 2H), 3.65 (s, 2H), 3.90 (t, J=6.1 Hz, 2H), 4.07-4.16 (m, 2H), 7.10 (t, J=7.9 Hz, 1H), 7.16 (d, J=8.6 Hz, 1H), 7.48 (dt, J=8.6, 1.8 Hz, 1H), 8.15 (dd, J=7.9, 1.2 Hz, 1H).

MS (ESI⁺) m/z: 355 (MH⁺).

HRMS (ESI⁺) for C₂₀H₂₇N₄O₂ (MH⁺): calcd, 355.21340. found, 355.21372.

### Step 3

### 6-(((1-(2-(6-Oxo-3,4-dihydro-2H-pyrimido[1,2-c]quinazolin-7(6H)-yl)ethyl)-2-oxabicyclo[2.2.2]octan-4-yl)amino)methyl)-2H-pyrido[3,2-b][1,4]oxazin-3(4H)-one

The title compound 6-(((1-(2-(6-oxo-3,4-dihydro-2H-pyrimido[1,2-c]quinazolin-7(6H)-yl)ethyl)-2-oxabicyclo[2.2.2]octan-4-yl)amino)methyl)-2H-pyrido[3,2-b][1,4]oxazin-3(4H)-one (25.0 mg) was prepared from 7-(2-(4-amino-2-oxabicyclo[2.2.2]octan-1-yl)ethyl)-3,4-dihydro-2H-pyrimido[1,2-c]quinazolin-6(7H)-one (51.5 mg) and I (27.3 mg) in the same manner as described for Step 3 of EXAMPLE 1.

¹H NMR (CDCl₃): δ 1.52-1.60 (m, 1H), 1.70-1.80 (m, 8H), 1.92-2.05 (m, 4H), 3.62 (t, J=5.5 Hz, 2H), 3.75 (s, 2H), 3.78 (s, 2H), 3.90 (t, J=6.1 Hz, 2H), 4.10-4.14 (m, 2H), 4.63 (s, 2H), 6.94 (d, J=7.9 Hz, 1H), 7.11 (t, J=7.3 Hz, 1H), 7.17 (d, J=8.6 Hz, 1H), 7.20 (d, J=7.9 Hz, 1H), 7.47 (t, J=7.3 Hz, 1H), 8.06 (brs, 1H), 8.16 (dd, J=7.9 Hz, 1H).

MS (ESI⁺) m/z: 517 (MH⁺).

HRMS (ESI⁺) for C₂₈H₃₃N₆O₄ (MH⁺): calcd, 517.25633. found, 517.25577.

### Example 119

### 6-((1-(2-(7-Fluoro-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one

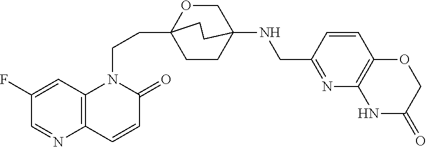 [SMILES: O=C1COc2ccc(CNC34CCC(CCn5c(=O)ccc6ncc(F)cc65)(CC3)OC4)nc2N1]

### Step 1

### tert-Butyl 1-(2-(7-Fluoro-2-oxo-1,5-naphthyridin-1(2H)-yl)ethyl)-2-oxabicyclo[2.2.2]octan-4-ylcarbamate

The title compound tert-butyl 1-(2-(7-fluoro-2-oxo-1,5-naphthyridin-1(2H)-yl)ethyl)-2-oxabicyclo[2.2.2]octan-4-ylcarbamate (125 mg) was prepared from 7-fluoro-1,5-naphthyridin-2(1H)-one (350 mg) and AC (745 mg) in the same manner as described for Step 1 of EXAMPLE 109.

¹H NMR (CDCl₃): δ 1.44 (s, 9H), 1.67-1.77 (m, 4H), 1.83-1.89 (m, 2H), 1.97-2.05 (m, 2H), 2.10-2.21 (m, 2H), 4.03 (s, 2H), 4.26-4.30 (m, 3H), 6.84 (d, J=9.8 Hz, 1H), 7.68 (dd, J=10.4, 2.4 Hz, 1H), 7.87 (d, J=9.8 Hz, 1H), 8.41 (d, J=2.4 Hz, 1H).

MS (ESI⁺) m/z: 418 (MH⁺).

HRMS (ESI⁺) for C₂₂H₂₉FN₃O₄ (MH⁺): calcd, 418.21421. found, 418.21453.

### Step 2

### 1-(2-(4-Amino-2-oxabicyclo[2.2.2]octan-1-yl)ethyl)-7-fluoro-1,5-naphthyridin-2(1H)-one

The title compound 1-(2-(4-amino-2-oxabicyclo[2.2.2]octan-1-yl)ethyl)-7-fluoro-1,5-naphthyridin-2(1H)-one (30.6 mg) was prepared from tert-butyl 1-(2-(7-fluoro-2-oxo-1,5-naphthyridin-1(2H)-yl)ethyl)-2-oxabicyclo[2.2.2]octan-4-ylcarbamate (40.0 mg) in the same manner as described for Step 2 of EXAMPLE 1.

¹H NMR (CDCl₃): δ 1.63-1.79 (m, 8H), 1.89-2.04 (m, 2H), 3.69 (s, 2H), 4.24-4.33 (m, 2H), 6.84 (d, J=9.8 Hz, 1H), 7.68 (dd, J=9.8, 1.8 Hz, 1H), 7.87 (d, J=9.8 Hz, 1H), 8.41 (d, J=1.8 Hz, 1H).

MS (ESI⁺) m/z: 318 (MH⁺).

HRMS (ESI⁺) for C₁₇H₂₁FN₃O₂ (MH⁺): calcd, 318.16178. found, 318.16160.

### Step 3

### 6-((1-(2-(7-Fluoro-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one

The title compound 6-((1-(2-(7-fluoro-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one (17.1 mg) was prepared from 1-(2-(4-amino-2-oxabicyclo[2.2.2]octan-1-yl)ethyl)-7-fluoro-1,5-naphthyridin-2(1H)-one (28.5 mg) and I (16.8 mg) in the same manner as described for Step 3 of EXAMPLE 1.

¹H NMR (CDCl₃): δ 1.70-1.86 (m, 8H), 1.94-2.04 (m, 2H), 3.76 (s, 2H), 3.82 (s, 2H), 4.27-4.32 (m, 2H), 4.64 (m, 2H), 6.85 (d, J=9.8 Hz, 1H), 6.95 (d, J=8.6 Hz, 1H), 7.21 (d, J=7.9 Hz, 1H), 7.67 (dd, J=9.8, 2.4 Hz, 1H), 7.88 (d, J=9.8 Hz, 1H), 7.91 (brs, 1H), 8.41 (d, J=2.4 Hz, 1H).

MS (ESI⁺) m/z: 480 (MH⁺).

HRMS (ESI⁺) for C₂₅H₂₇FN₅O₄ (MH⁺): calcd, 480.20471. found, 480.20433.

### Example 120

### 6-(((1-(2-(7-Methoxy-2-oxo-1,5-naphthyridin-1(2H)-yl)ethyl)-2-oxabicyclo[2.2.2]octan-4-yl)amino)methyl)-2H-pyrido[3,2-b][1,4]oxazin-3(4H)-one

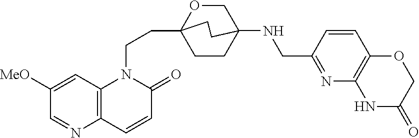 [SMILES: COc1cnc2ccc(=O)n(CCC34CCC(NCc5ccc6c(n5)NC(=O)CO6)(CC3)CO4)c2c1]

### Step 1

### tert-Butyl (1-(2-(7-Methoxy-2-oxo-1,5-naphthyridin-1(2H)-yl)ethyl)-2-oxabicyclo[2.2.2]octan-4-yl)carbamate

To a suspension of tert-butyl (1-(2-(7-fluoro-2-oxo-1,5-naphthyridin-1(2H)-yl)ethyl)-2-oxabicyclo[2.2.2]octan-4-yl)carbamate (40.0 mg) in methanol (0.24 mL) was added a solution of sodium methoxide (0.21 g, 25 wt % in methanol), the mixture was stirred at room temperature for 1.5 hours. After dilution of the mixture with dichloromethane, the mixture was washed with water and brine. The organic extracts were dried over anhydrous sodium sulfate, filtered, and then concentrated in vacuo. Flash chromatography (silica, ethyl acetate) of the residue gave tert-butyl (1-(2-(7-methoxy-2-oxo-1,5-naphthyridin-1(2H)-yl)ethyl)-2-oxabicyclo[2.2.2]octan-4-yl)carbamate (40.0 mg).

¹H NMR (CDCl₃): δ 1.43 (s, 9H), 1.69-1.87 (m, 6H), 1.91-2.06 (m, 2H), 2.09-2.22 (m, 2H), 3.96 (s, 3H), 4.07 (s, 2H), 4.26-4.41 (m, 3H), 6.71 (d, J=9.8 Hz, 1H), 7.53 (d, J=1.8 Hz, 1H), 7.83 (d, J=9.8 Hz, 1H), 8.26 (d, J=2.4 Hz, 1H).

MS (ESI⁺) m/z: 430 (MH⁺).

HRMS (ESI⁺) for C₂₃H₃₂N₃O₅ (MH⁺): calcd, 430.23420. found, 430.23361.

### Step 2

### 1-(2-(4-Amino-2-oxabicyclo[2.2.2]octan-1-yl)ethyl)-7-methoxy-1,5-naphthyridin-2(1H)-one

The title compound 1-(2-(4-amino-2-oxabicyclo[2.2.2]octan-1-yl)ethyl)-7-methoxy-1,5-naphthyridin-2(1H)-one (25.0 mg) was prepared from tert-butyl (1-(2-(7-methoxy-2-oxo-1,5-naphthyridin-1(2H)-yl)ethyl)-2-oxabicyclo[2.2.2]octan-4-yl)carbamate (35.0 mg) in the same manner as described for Step 2 of EXAMPLE 1.

¹H NMR (CDCl₃): δ 1.25 (m, 2H), 1.64-1.80 (m, 8H), 1.94-1.98 (m, 2H), 3.68 (s, 2H), 3.97 (s, 3H), 4.32-4.36 (m, 2H), 6.72 (d, J=9.8 Hz, 1H), 7.55 (d, J=2.4 Hz, 1H), 7.83 (d, J=9.8 Hz, 1H), 8.27 (d, J=2.4 Hz, 1H).

MS (ESI⁺) m/z: 330 (MH⁺).

HRMS (ESI⁺) for C₁₈H₂₄N₃O₃ (MH⁺): calcd, 330.18177. found, 330.18208.

### Step 3

### 6-(((1-(2-(7-Methoxy-2-oxo-1,5-naphthyridin-1(2H)-yl)ethyl)-2-oxabicyclo[2.2.2]octan-4-yl)amino)methyl)-2H-pyrido[3,2-b][1,4]oxazin-3(4H)-one

The title compound 6-(((1-(2-(7-methoxy-2-oxo-1,5-naphthyridin-1(2H)-yl)ethyl)-2-oxabicyclo[2.2.2]octan-4-yl)amino)methyl)-2H-pyrido[3,2-b][1,4]oxazin-3(4H)-one (18.6 mg) was prepared from 1-(2-(4-amino-2-oxabicyclo[2.2.2]octan-1-yl)ethyl)-7-methoxy-1,5-naphthyridin-2(1H)-one (23.0 mg) and I (13.1 mg) in the same manner as described for Step 3 of EXAMPLE 1.

¹H NMR (CDCl₃): δ 1.69-1.88 (m, 8H), 1.97-2.05 (m, 2H), 3.75 (s, 2H), 3.81 (s, 2H), 3.97 (s, 3H), 4.33-4.37 (m, 2H), 4.64 (s, 2H), 6.73 (d, J=9.8 Hz, 1H), 6.94 (d, J=7.9 Hz, 1H), 7.21 (d, J=8.6 Hz, 1H), 7.54 (d, J=2.4 Hz, 1H), 7.84 (d, J=9.2 Hz, 1H), 7.99 (br, 1H), 8.27 (d, J=1.8 Hz, 1H).

MS (ESI⁺) m/z: 492 (MH⁺).

HRMS (ESI⁺) for C₂₆H₃₀N₅O₅ (MH⁺): calcd, 492.22469. found, 492.22400.

### Example 121

### 6-(((1-(2-(7-Methoxy-2-oxopyrido[2,3-b]pyrazin-1(2H)-yl)ethyl)-2-oxabicyclo[2.2.2]octan-4-yl)amino)methyl)-2H-pyrido[3,2-b][1,4]oxazin-3(4H)-one

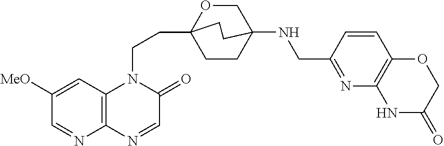 [SMILES: COc1cnc2ncc(=O)n(CCC34CCC(NCc5ccc6c(n5)NC(=O)CO6)(CC3)CO4)c2c1]

### Step 1

### tert-Butyl (1-(2-(6-Methoxy-3-oxopyrido[2,3-b]pyrazin-4(3H)-yl)ethyl)-2-oxabicyclo[2.2.2]octan-4-yl)carbamate

The title compound tert-butyl (1-(2-(6-methoxy-3-oxopyrido[2,3-b]pyrazin-4(3H)-yl)ethyl)-2-oxabicyclo[2.2.2]octan-4-yl)carbamate (40.2 mg) was prepared from 7-methoxypyrido[2,3-b]pyrazin-2(1H)-one (250 mg) and AC (493 mg) in the same manner as described for Step 1 of EXAMPLE 109.

¹H NMR (DMSO-d₆): δ 1.35 (s, 9H), 1.62-2.01 (m, 10H), 3.76 (s, 2H), 3.99 (s, 3H), 4.26-4.39 (m, 2H), 6.57 (brs, 1H), 6.82 (d, J=8.6 Hz, 1H), 8.08 (s, 1H), 8.11 (d, J=8.6 Hz, 1H).

MS (ESI⁺) m/z: 431 (MH⁺).

HRMS (ESI⁺) for C₂₂H₃₁N₄O₅ (MH⁺): calcd, 431.22944. found, 431.22954.

### Step 2

### 4-(2-(4-Amino-2-oxabicyclo[2.2.2]octan-1-yl)ethyl)-6-methoxypyrido[2,3-b]pyrazin-3(4H)-one

The title compound 4-(2-(4-amino-2-oxabicyclo[2.2.2]octan-1-yl)ethyl)-6-methoxypyrido[2,3-b]pyrazin-3(4H)-one (83.0 mg) was prepared from tert-butyl (1-(2-(6-methoxy-3-oxopyrido[2,3-b]pyrazin-4(3H)-yl)ethyl)-2-oxabicyclo[2.2.2]octan-4-yl)carbamate (116 mg) in the same manner as described for Step 2 of EXAMPLE 32.

¹H NMR (DMSO-d₆): δ 1.50-1.60 (m, 6H), 1.64-1.71 (m, 4H), 1.81-1.94 (m, 2H), 3.43 (s, 2H), 3.98 (s, 3H), 4.31-4.35 (m, 2H), 6.83 (d, J=8.6 Hz, 1H), 8.08 (s, 1H), 8.11 (d, J=8.6 Hz, 1H).

### Step 3

### 6-(((1-(2-(7-M ethoxy-2-oxopyrido[2,3-b]pyrazin-1(2H)-yl)ethyl)-2-oxabicyclo[2.2.2]octan-4-yl)amino)methyl)-2H-pyrido[3,2-b][1,4]oxazin-3(4H)-one

The title compound 6-(((1-(2-(7-methoxy-2-oxopyrido[2,3-b]pyrazin-1(2H)-yl)ethyl)-2-oxabicyclo[2.2.2]octan-4-yl)amino)methyl)-2H-pyrido[3,2-b][1,4]oxazin-3(4H)-one (121 mg) was prepared from 4-(2-(4-amino-2-oxabicyclo[2.2.2]octan-1-yl)ethyl)-6-methoxypyrido[2,3-b]pyrazin-3(4H)-one (108 mg) and I (61.2 mg) in the same manner as described for Step 3 of EXAMPLE 1.

¹H NMR (DMSO-d₆): δ 1.59-1.70 (m, 8H), 1.88-1.90 (m, 3H), 3.56 (s, 2H), 3.61 (d, J=4.3 Hz, 2H), 3.99 (s, 3H), 4.30-4.38 (m, 2H), 4.59 (s, 2H), 6.83 (d, J=8.6 Hz, 1H), 7.00 (d, J=7.9 Hz, 1H), 7.27 (d, J=8.6 Hz, 1H), 8.08 (s, 1H), 8.12 (d, J=9.2 Hz, 1H), 11.15 (s, 1H).

MS (ESI⁺) m/z: 493 (MH⁺).

HRMS (ESI⁺) for C₂₅H₂₉N₆O₅ (MH⁺): calcd, 493.21994. found, 493.22015.

### Example 122

### 4-(2-(4-((2,3-Dihydro-[1,4]dioxino[2,3-c]pyridin-7-yl)methylamino)-2-oxabicyclo[2.2.2]octan-1-yl)ethyl)-6-methoxypyrido[3,2-b]pyrazin-3(4H)-one

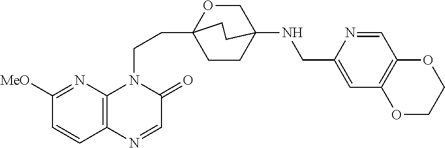 [SMILES: COc1ccc2ncc(=O)n(CCC34CCC(NCc5cc6c(cn5)OCCO6)(CC3)CO4)c2n1]

The title compound 4-(2-(4-((2,3-dihydro-[1,4]dioxino[2,3-c]pyridin-7-yl)methylamino)-2-oxabicyclo[2.2.2]octan-1-yl)ethyl)-6-methoxypyrido[3,2-b]pyrazin-3(4H)-one (30.5 mg) was prepared from 4-(2-(4-amino-2-oxabicyclo[2.2.2]octan-1-yl)ethyl)-6-methoxypyrido[3,2-b]pyrazin-3(4H)-one (40.0 mg) and 2,3-dihydro-[1,4]dioxino[2,3-c]pyridine-7-carbaldehyde (22.0 mg) in the same manner as described for Step 3 of EXAMPLE 1.

¹H NMR (CDCl₃): δ 1.70-1.91 (m, 8H), 1.95-2.11 (m, 2H), 3.71 (s, 2H), 3.73 (s, 2H), 4.05 (s, 3H), 4.26-4.33 (m, 4H), 4.45-4.52 (m, 2H), 6.71 (d, J=8.6 Hz, 1H), 6.82 (s, 1H), 8.00 (d, J=8.6 Hz, 1H), 8.08 (s, 1H), 8.14 (s, 1H).

MS (ESI⁺) m/z: 480 (MH⁺).

HRMS (ESI⁺) for C₂₅H₃₀N₅O₅ (MH⁺): calcd, 480.22469. found, 480.22484.

### Example 123

### 6-Methoxy-4-(2-(4-((1-methyl-2-oxo-1,2-dihydroquinolin-3-yl)methylamino)-2-oxabicyclo[2.2.2]octan-1-yl)ethyl)pyrido[3,2-b]pyrazin-3(4H)-one

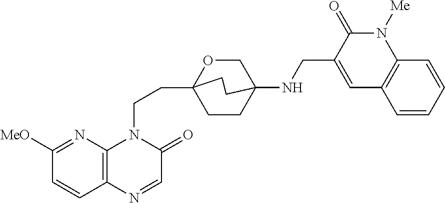 [SMILES: COc1ccc2ncc(=O)n(CCC34CCC(NCc5cc6ccccc6n(C)c5=O)(CC3)CO4)c2n1]

The title compound 6-methoxy-4-(2-(4-((1-methyl-2-oxo-1,2-dihydroquinolin-3-yl)methylamino)-2-oxabicyclo[2.2.2]octan-1-yl)ethyl)pyrido[3,2-b]pyrazin-3(4H)-one (47.6 mg) was prepared from 4-(2-(4-amino-2-oxabicyclo[2.2.2]octan-1-yl)ethyl)-6-methoxypyrido[3,2-b]pyrazin-3(4H)-one (40.0 mg) and 1-methyl-2-oxo-1,2-dihydroquinoline-3-carbaldehyde (25.0 mg) in the same manner as described for Step 3 of EXAMPLE 1.

¹H NMR (CDCl₃): δ 1.66-1.88 (m, 8H), 2.02-2.08 (m, 2H), 3.73 (s, 2H), 3.74 (s, 3H), 3.79 (s, 2H), 4.06 (s, 3H), 4.49-4.54 (m, 2H), 6.72 (d, J=8.6 Hz, 1H), 7.23 (d, J=8.6 Hz, 1H), 7.36 (d, J=7.9 Hz, 1H), 7.51-7.59 (m, 2H), 7.74 (s, 1H), 8.00 (d, J=8.6 Hz, 1H), 8.14 (s, 1H).

MS (ESI⁺) m/z: 502 (MH⁺).

HRMS (ESI⁺) for C₂₈H₃₂N₅O₄ (MH⁺): calcd, 502.24543. found, 502.24473.

### Example 124

### 6-(((1-(2-(3-Hydroxy-6-methoxy-1,5-naphthyridin-4-yl)ethyl)-2-oxabicyclo[2.2.2]octan-4-yl)amino)methyl)-2H-pyrido[3,2-b][1,4]oxazin-3(4H)-one

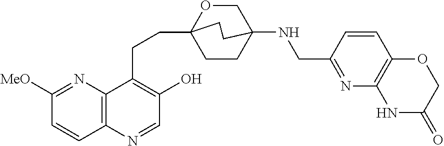 [SMILES: COc1ccc2ncc(O)c(CCC34CCC(NCc5ccc6c(n5)NC(=O)CO6)(CC3)CO4)c2n1]

### Step 1

### tert-Butyl (1-(2-(3-(Benzyloxy)-6-methoxy-1,5-naphthyridin-4-yl)ethyl)-2-oxabicyclo[2.2.2]octan-4-yl)carbamate

To a suspension of sodium hydride (8.0 mg, 50% in mineral oil) in N-methyl-2-pyrrolidone (0.5 mL) was added benzyl alcohol (19.2 mL), the mixture was stirred at room temperature for 30 minutes. tert-butyl 1-(2-(3-fluoro-6-methoxy-1,5-naphthyridin-4-yl)ethyl)-2-oxabicyclo[2.2.2]octan-4-ylcarbamate (Example 18, Step 2, 40 mg) was added to the mixture, the resulting mixture was stirred at the same temperature for 3 hours. After dilution of the mixture with dichloromethane, the reaction was quenched by adding 1 N hydrochloric acid. The organic extracts were washed with water and brine, dried over anhydrous sodium sulfate, filtered, and then concentrated in vacuo. Flash chromatography (silica, hexane:ethyl acetate=2:1) of the residue gave tert-butyl (1-(2-(3-(benzyloxy)-6-methoxy-1,5-naphthyridin-4-yl)ethyl)-2-oxabicyclo[2.2.2]octan-4-yl)carbamate (24.6 mg).

¹H NMR (CDCl₃): δ 1.44 (s, 9H), 1.70-2.17 (m, 10H), 3.16-3.24 (m, 2H), 3.97 (s, 2H), 4.06 (s, 3H), 4.27 (br, 1H), 5.31 (s, 2H), 6.96 (d, J=9.2 Hz, 1H), 7.30-7.42 (m, 3H), 7.49 (d, J=7.3 Hz, 2H), 8.09 (d, J=9.2 Hz, 1H), 8.58 (s, 1H).

MS (ESI⁺) m/z: 520 (MH⁺).

HRMS (ESI⁺) for C₃₀H₃₈N₃O₅ (MH⁺): calcd, 520.28115. found, 520.28170.

### Step 2

### tert-Butyl (1-(2-(3-Hydroxy-6-methoxy-1,5-naphthyridin-4-yl)ethyl)-2-oxabicyclo[2.2.2]octan-4-yl)carbamate

A suspension of tert-butyl 1-(2-(3-(benzyloxy)-6-methoxy-1,5-naphthyridin-4-yl)ethyl)-2-oxabicyclo[2.2.2]octan-4-ylcarbamate (155 mg), 10% Pd—C (75.5 mg) in methanol (3.0 mL) was stirred at room temperature for 3 hours under H₂ atmosphere (1 kg/cm²). After the insoluble materials were filtered off, the filtrate was concentrated in vacuo. Flash chromatography (silica, hexane:ether=1:1) of the residue gave tert-butyl (1-(2-(3-hydroxy-6-methoxy-1,5-naphthyridin-4-yl)ethyl)-2-oxabicyclo[2.2.2]octan-4-yl)carbamate (89.6 mg).

¹H NMR (CDCl₃): δ 1.43 (s, 9H), 1.62-1.66 (m, 2H), 1.78-1.86 (m, 4H), 1.90-2.01 (m, 2H), 2.10-2.20 (m, 2H), 3.23 (t, J=6.1 Hz, 2H), 4.05 (s, 3H), 4.15 (s, 2H), 4.31 (br, 1H), 6.92 (d, J=9.2 Hz, 1H), 8.10 (d, J=8.6 Hz, 1H), 8.56 (s, 1H).

MS (ESI⁺) m/z: 430 (MH⁺).

HRMS (ESI⁺) for C₂₃H₃₁N₃O₅ (MH⁺): calcd, 430.23420. found, 430.23467.

### Step 3

### 4-(2-(4-Amino-2-oxabicyclo[2.2.2]octan-1-yl)ethyl)-6-methoxy-1,5-naphthyridin-3-ol

The title compound 4-(2-(4-amino-2-oxabicyclo[2.2.2]octan-1-yl)ethyl)-6-methoxy-1,5-naphthyridin-3-ol (35.7 mg) was prepared from tert-butyl (1-(2-(3-hydroxy-6-methoxy-1,5-naphthyridin-4-yl)ethyl)-2-oxabicyclo[2.2.2]octan-4-yl)carbamate (51.0 mg) in the same manner as described for Step 2 of EXAMPLE 32.

¹H NMR (CDCl₃): δ 1.55-2.05 (m, 10H), 3.23 (t, J=6.4 Hz, 2H), 3.78 (s, 2H), 4.05 (s, 3H), 6.92 (d, J=8.6 Hz, 1H), 8.10 (d, J=9.2 Hz, 1H), 8.50 (s, 1H).

MS (ESI⁺) m/z: 330 (MH⁺).

HRMS (ESI⁺) for C₁₈H₂₄N₃O₃ (MH⁺): calcd, 330.18177. found, 330.18172.

### Step 4

### 6-(((1-(2-(3-Hydroxy-6-methoxy-1,5-naphthyridin-4-yl)ethyl)-2-oxabicyclo[2.2.2]octan-4-yl)amino)methyl)-2H-pyrido[3,2-b][1,4]oxazin-3(4H)-one

The title compound 6-(((1-(2-(3-hydroxy-6-methoxy-1,5-naphthyridin-4-yl)ethyl)-2-oxabicyclo[2.2.2]octan-4-yl)amino)methyl)-2H-pyrido[3,2-b][1,4]oxazin-3(4H)-one (11.0 mg) was prepared from 4-(2-(4-amino-2-oxabicyclo[2.2.2]octan-1-yl)ethyl)-6-methoxy-1,5-naphthyridin-3-ol (33.0 mg) and I (18.7 mg) in the same manner as described for Step 3 of EXAMPLE 1.

¹H NMR (DMSO-d₆): δ 1.54-1.86 (m, 10H), 3.00-3.05 (m, 2H), 3.58 (br, 2H), 3.63 (br, 2H), 3.98 (s, 3H), 4.59 (s, 2H), 6.97 (d, J=9.2 Hz, 1H), 7.01 (d, J=7.9 Hz, 1H), 7.28 (d, J=8.6 Hz, 1H), 8.08 (d, J=9.2 Hz, 1H), 8.43 (s, 1H), 10.16 (s, 1H), 11.15 (s, 1H).

MS (ESI⁺) m/z: 492 (MH⁺).

HRMS (ESI⁺) for C₂₆H₃₀N₅O₅ (MH⁺): calcd, 492.22469. found, 492.22434.

### Example 125

### 6-((1-(2-(3-Fluoro-6-methoxyquinolin-4-yl)ethyl)-2-oxabicyclo[2.2.2]octan-4-ylamino)methyl)-2H-pyrido[3,2-b][1,4]oxazin-3(4H)-one Hydrochloride

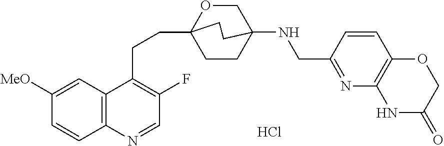 [SMILES: COc1ccc2ncc(F)c(CCC34CCC(NCc5ccc6c(n5)NC(=O)CO6)(CC3)CO4)c2c1 ; Cl]

### Step 1

### tert-Butyl 1-(2-(3-Fluoro-6-methoxyquinolin-4-yl)ethyl)-2-oxabicyclo[2.2.2]octan-4-ylcarbamate

The title compound tert-butyl 1-(2-(3-fluoro-6-methoxyquinolin-4-yl)ethyl)-2-oxabicyclo[2.2.2]octan-4-ylcarbamate (259 mg) was prepared from B (500 mg) and 4-bromo-3-fluoro-6-methoxyquinoline (504 mg) in the same manner as described for Step 1 of EXAMPLE 17.

¹H NMR (CDCl₃): δ 1.44 (s, 9H), 1.65-1.78 (m, 4H), 1.81-1.92 (m, 2H), 1.98-2.20 (m, 4H), 3.02-3.11 (m, 2H), 3.95 (s, 3H), 4.02 (s, 2H), 4.31 (br, 1H), 7.29 (dd, J=9.2, 2.4 Hz, 1H), 7.34 (d, J=3.1 Hz, 1H), 7.96 (d, J=9.2 Hz, 1H), 8.56 (s, 1H).

MS (ESI⁺) m/z: 431 (MH⁺).

HRMS (ESI⁺) for C₂₄H₃₂FN₂O₄ (MH⁺): calcd, 431.23461. found, 431.23415.

### Step 2

### 1-(2-(3-Fluoro-6-methoxyquinolin-4-yl)ethyl)-2-oxabicyclo[2.2.2]octan-4-amine

The title compound 1-(2-(3-fluoro-6-methoxyquinolin-4-yl)ethyl)-2-oxabicyclo[2.2.2]octan-4-amine (143 mg) was prepared from tert-butyl 1-(2-(3-fluoro-6-methoxyquinolin-4-yl)ethyl)-2-oxabicyclo[2.2.2]octan-4-ylcarbamate (200 mg) in the same manner as described for Step 2 of EXAMPLE 32.

¹H NMR (DMSO-d₆): δ 1.41 (br, 2H), 1.49-1.74 (m, 8H), 1.79-1.91 (m, 2H), 2.97-3.05 (m, 2H), 3.51 (s, 2H), 3.92 (s, 3H), 7.33 (d, J=3.0 Hz, 1H), 7.37 (dd, J=9.1, 3.0 Hz, 1H), 7.93 (d, J=9.1 Hz, 1H), 8.65 (d, J=1.2 Hz, 1H).

MS (ESI⁺) m/z: 331 (MH⁺).

HRMS (ESI⁺) for C₁₉H₂₄FN₂O₂ (MH⁺): calcd, 331.18218. found, 331.18189.

### Step 3

### 6-(((1-(2-(3-Fluoro-6-methoxyquinolin-4-yl)ethyl)-2-oxabicyclo[2.2.2]octan-4-yl)amino)methyl)-2H-pyrido[3,2-b][1,4]oxazin-3(4H)-one

The title compound 6-(((1-(2-(3-fluoro-6-methoxyquinolin-4-yl)ethyl)-2-oxabicyclo[2.2.2]octan-4-yl)amino)methyl)-2H-pyrido[3,2-b][1,4]oxazin-3(4H)-one (98.1 mg) was prepared from 1-(2-(3-fluoro-6-methoxyquinolin-4-yl)ethyl)-2-oxabicyclo[2.2.2]octan-4-amine (83.0 mg) and I (49.2 mg) in the same manner as described for Step 3 of EXAMPLE 1.

¹H NMR (DMSO-d₆): δ 1.55-1.78 (m, 8H), 1.82-1.97 (m, 3H), 2.97-3.07 (m, 2H), 3.64 (s, 4H), 3.92 (s, 3H), 4.59 (s, 2H), 7.02 (d, J=7.9 Hz, 1H), 7.28 (d, J=7.9 Hz, 1H), 7.33 (d, J=2.4 Hz, 1H), 7.37 (dd, J=9.2, 3.1 Hz, 1H), 7.93 (d, J=9.2 Hz, 1H), 8.65 (s, 1H), 11.15 (s, 1H).

MS (ESI⁺) m/z: 493 (MH⁺).

HRMS (ESI⁺) for C₂₇H₃₀FN₄O₄ (MH⁺): calcd, 493.22511. found, 493.22535.

### Step 4

### 6-(((1-(2-(3-Fluoro-6-methoxyquinolin-4-yl)ethyl)-2-oxabicyclo[2.2.2]octan-4-yl)amino)methyl)-2H-pyrido[3,2-b][1,4]oxazin-3(4H)-one Hydrochloride

The title compound 6-(((1-(2-(3-fluoro-6-methoxyquinolin-4-yl)ethyl)-2-oxabicyclo[2.2.2]octan-4-yl)amino)methyl)-2H-pyrido[3,2-b][1,4]oxazin-3(4H)-one hydrochloride (83.5 mg) was prepared from 6-(((1-(2-(3-fluoro-6-methoxyquinolin-4-yl)ethyl)-2-oxabicyclo[2.2.2]octan-4-yl)amino)methyl)-2H-pyrido[3,2-b][1,4]oxazin-3(4H)-one (85.0 mg) in the same manner as described for Step 4 of EXAMPLE 3.

¹H NMR (DMSO-d₆): δ 1.61-1.71 (m, 2H), 1.79-1.92 (m, 2H), 1.95-2.11 (m, 6H), 2.98-3.09 (m, 2H), 3.93 (s, 3H), 3.97 (s, 2H), 4.12 (t, J=6.1 Hz, 2H), 4.69 (s, 2H), 7.22 (d, J=7.9 Hz, 1H), 7.31 (d, J=2.4 Hz, 1H), 7.39 (dd, J=9.2, 2.4 Hz, 1H), 7.46 (d, J=7.9 Hz, 1H), 7.95 (d, J=9.2 Hz, 1H), 8.67 (s, 1H), 9.29 (br, 2H), 11.32 (s, 1H).

MS (ESI⁺) m/z: 493 (MH⁺) (as free base).

HRMS (ESI⁺) for C₂₇H₃₀FN₄O₄ (MH⁺) (as free base): calcd, 493.22511. found, 493.22448.

### Example 126

### 6-(((1-(2-(6-Methyl-3-oxopyrido[2,3-b]pyrazin-4(3H)-yl)ethyl)-2-oxabicyclo[2.2.2]octan-4-yl)amino)methyl)-2H-pyrido[3,2-b][1,4]oxazin-3(4H)-one

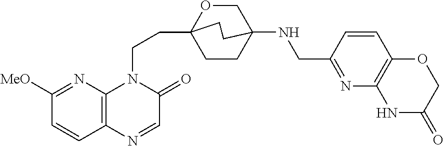 [SMILES: COc1ccc2ncc(=O)n(CCC34CCC(NCc5ccc6c(n5)NC(=O)CO6)(CC3)CO4)c2n1]

### Step 1

### tert-Butyl 1-(2-(6-Methyl-3-oxopyrido[3,2-b]pyrazin-4(3H)-yl)ethyl)-2-oxabicyclo[2.2.2]octan-4-ylcarbamate

The title compound tert-butyl 1-(2-(6-methyl-3-oxopyrido[3,2-b]pyrazin-4(3H)-yl)ethyl)-2-oxabicyclo[2.2.2]octan-4-ylcarbamate (66.4 mg) was prepared from 6-methylpyrido[3,2-b]pyrazin-3(4H)-one (50.0 mg) and AC (108 mg) in the same manner as described for Step 1 of EXAMPLE 109.

¹H NMR (CDCl₃): δ 1.43 (s, 9H), 1.75-1.88 (m, 6H), 2.05-2.17 (m, 4H), 2.65 (s, 3H), 3.91 (s, 2H), 4.27 (br, 1H), 4.49-4.53 (m, 2H), 7.14 (d, J=7.9 Hz, 1H), 8.01 (d, J=7.9 Hz, 1H), 8.23 (s, 1H).

MS (ESI⁺) m/z: 415 (MH⁺).

HRMS (ESI⁺) for C₂₂H₃₁N₄O₄ (MH⁺): calcd, 415.23453. found, 415.23523.

### Step 2

### 4-(2-(4-Amino-2-oxabicyclo[2.2.2]octan-1-yl)ethyl)-6-methylpyrido[3,2-b]pyrazin-3(4H)-one

The title compound 4-(2-(4-amino-2-oxabicyclo[2.2.2]octan-1-yl)ethyl)-6-methylpyrido[3,2-b]pyrazin-3(4H)-one (116 mg) was prepared from tert-butyl 1-(2-(6-methyl-3-oxopyrido[3,2-b]pyrazin-4(3H)-yl)ethyl)-2-oxabicyclo[2.2.2]octan-4-ylcarbamate (73.2 mg) was prepared from 6-methylpyrido[3,2-b]pyrazin-3(4H)-one (160 mg) in the same manner as described for Step 2 of EXAMPLE 32.

¹H NMR (CDCl₃): δ 1.75-1.88 (m, 8H), 2.02-2.20 (m, 2H), 2.65 (s, 3H), 3.61 (s, 2H), 4.50-4.54 (m, 2H), 7.14 (d, J=7.9 Hz, 1H), 8.01 (d, J=7.9 Hz, 1H), 8.24 (s, 1H).

### Step 3

### 6-(((1-(2-(6-Methyl-3-oxopyrido[2,3-b]pyrazin-4(3H)-yl)ethyl)-2-oxabicyclo[2.2.2]octan-4-yl)amino)methyl)-2H-pyrido[3,2-b][1,4]oxazin-3(4H)-one

The title compound 6-(((1-(2-(6-methyl-3-oxopyrido[2,3-b]pyrazin-4(3H)-yl)ethyl)-2-oxabicyclo[2.2.2]octan-4-yl)amino)methyl)-2H-pyrido[3,2-b][1,4]oxazin-3(4H)-one (73.2 mg) was prepared from 4-(2-(4-amino-2-oxabicyclo[2.2.2]octan-1-yl)ethyl)-6-methylpyrido[3,2-b]pyrazin-3(4H)-one (110 mg) and I (68.5 mg) in the same manner as described for Step 3 of EXAMPLE 1.

¹H NMR (CDCl₃): δ 1.74-1.90 (m, 8H), 2.08-2.17 (m, 2H), 2.65 (s, 3H), 3.75 (s, 2H), 3.79 (s, 2H), 4.51-4.55 (m, 2H), 4.63 (s, 2H), 6.95 (d, J=8.6 Hz, 1H), 7.14 (d, J=7.9 Hz, 1H), 7.20 (d, J=7.9 Hz, 1H), 8.01 (d, J=7.9 Hz, 1H), 8.24 (s, 1H).

MS (ESI⁺) m/z: 477 (MH⁺).

HRMS (ESI⁺) for C₂₅H₂₉N₆O₄ (MH⁺): calcd, 477.22503. found, 477.22492.

### Example 127

### Methyl 6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridine-3-carboxylate Hydrochloride

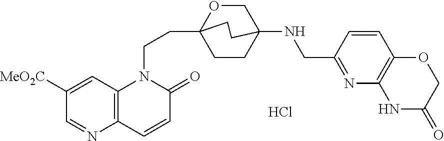 [SMILES: COC(=O)c1cnc2ccc(=O)n(CCC34CCC(NCc5ccc6c(n5)NC(=O)CO6)(CC3)CO4)c2c1 ; Cl]

### Step 1

### Methyl 5-(2-(4-(tert-Butoxycarbonylamino)-2-oxabicyclo[2.2.2]octan-1-yl)ethyl)-6-oxo-5,6-dihydro-1,5-naphthyridine-3-carboxylate

A mixture of methyl 6-oxo-5,6-dihydro-1,5-naphthyridine-3-carboxylate dihydrobromide (100 mg), potassium carbonate (137 mg) and 18-crown-6 (72.2 mg) in 1,4-dioxane (1.4 mL) was stirred at room temperature for 30 minutes. To the mixture was added a solution of AD (104 mg) in 1,4-dioxane (1.4 mL), the mixture was stirred at 80° C. for 16 hours and concentrated in vacuo. After dilution of the residue with water and saturated ammonium chloride solution, the mixture was extracted with ethyl acetate. The organic extracts were dried over anhydrous sodium sulfate, filtered, and then concentrated in vacuo. Flash chromatography (silica, hexane:ethyl acetate=1:2) of the residue gave methyl 5-(2-(4-(tert-butoxycarbonylamino)-2-oxabicyclo[2.2.2]octan-1-yl)ethyl)-6-oxo-5,6-dihydro-1,5-naphthyridine-3-carboxylate (47.8 mg).

¹H NMR (CDCl₃): δ 1.44 (s, 9H), 1.71-2.18 (m, 10H), 4.02 (s, 3H), 4.05 (s, 2H), 4.30 (s, 1H), 4.34-4.41 (m, 2H), 6.99 (d, J=9.8 Hz, 1H), 7.93 (d, J=9.8 Hz, 1H), 8.58 (s, 1H), 9.10 (d, J=1.8 Hz, 1H).

MS (ESI⁺) m/z: 458 (MH⁺).

HRMS (ESI⁺) for C₂₄H₃₂N₃O₆ (MH⁺): calcd, 458.22911. found, 458.22873.

### Step 2

### Methyl 5-(2-(4-Amino-2-oxabicyclo[2.2.2]octan-1-yl)ethyl)-6-oxo-5,6-dihydro-1,5-naphthyridine-3-carboxylate

The title compound methyl 5-(2-(4-amino-2-oxabicyclo[2.2.2]octan-1-yl)ethyl)-6-oxo-5,6-dihydro-1,5-naphthyridine-3-carboxylate (140 mg) was prepared from methyl 5-(2-(4-(tert-butoxycarbonylamino)-2-oxabicyclo[2.2.2]octan-1-yl)ethyl)-6-oxo-5,6-dihydro-1,5-naphthyridine-3-carboxylate (200 mg) in the same manner as described for Step 2 of EXAMPLE 1.

¹H NMR (CDCl₃): δ 1.44-1.81 (m, 12H), 3.72 (s, 2H), 4.03 (s, 3H), 4.35-4.41 (m, 2H), 6.99 (d, J=9.8 Hz, 1H), 7.93 (d, J=9.8 Hz, 1H), 8.60 (d, J=1.2 Hz, 1H), 9.09 (d, J=1.2 Hz, 1H).

MS (ESI⁺) m/z: 358 (MH⁺).

HRMS (ESI⁺) for C₁₉H₂₄N₃O₄ (MH⁺): calcd, 358.17668. found, 358.17738.

### Step 3

### Methyl 6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridine-3-carboxylate

The title compound methyl 6-oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridine-3-carboxylate (108 mg) was prepared from methyl 5-(2-(4-amino-2-oxabicyclo[2.2.2]octan-1-yl)ethyl)-6-oxo-5,6-dihydro-1,5-naphthyridine-3-carboxylate (140 mg) and I (60.2 mg) in the same manner as described for Step 3 of EXAMPLE 1.

¹H NMR (DMSO-d₆): δ 1.57-1.97 (m, 11H), 3.65 (m, 4H), 3.93 (s, 3H), 4.22-4.30 (m, 2H), 4.59 (s, 2H), 6.99 (d, J=9.8 Hz, 1H), 7.02 (d, J=8.0 Hz, 1H), 7.28 (d, J=8.0 Hz, 1H), 8.00 (d, J=9.8 Hz, 1H), 8.42 (s, 1H), 8.97 (d, J=1.2 Hz, 1H), 11.16 (s, 1H).

MS (ESI⁺) m/z: 520 (MH⁺).

HRMS (ESI⁺) for C₂₇H₃₀N₅O₆ (MH⁺): calcd, 520.21961. found, 520.21964.

### Step 4

### Methyl 6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridine-3-carboxylate Hydrochloride

The title compound methyl 6-oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridine-3-carboxylate hydrochloride (43.8 mg) was prepared from methyl 6-oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridine-3-carboxylate (55.0 mg) in the same manner as described for Step 4 of EXAMPLE 3.

¹H NMR (DMSO-d₆): δ 1.64-2.10 (m, 10H), 3.92-4.02 (m, 5H), 4.13 (brs, 2H), 4.24-4.32 (m, 2H), 4.68 (s, 2H), 7.00 (d, J=9.8 Hz, 1H), 7.20 (d, J=8.0 Hz, 1H), 7.45 (d, J=7.9 Hz, 1H), 8.01 (d, J=9.8 Hz, 1H), 8.40 (s, 1H), 8.98 (d, J=1.8 Hz, 1H), 9.28 (s, 1H), 11.33 (s, 1H).

MS (ESI⁺) m/z: 520 (MH⁺) (as free base).

HRMS (ESI⁺) for C₂₇H₃₀N₅O₆ (MH⁺) (as free base): calcd, 520.21961. found, 520.22054.

### Example 128

### 6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridine-3-carbonitrile

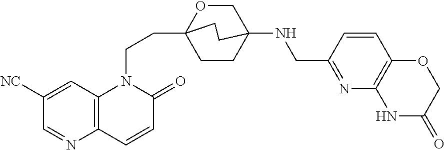 [SMILES: N#Cc1cnc2ccc(=O)n(CCC34CCC(NCc5ccc6c(n5)NC(=O)CO6)(CC3)CO4)c2c1]

### Step 1

### tert-Butyl 1-(2-(7-Bromo-2-oxo-1,5-naphthyridin-1(2H)-yl)ethyl)-2-oxabicyclo[2.2.2]octan-4-ylcarbamate

The title compound tert-butyl 1-(2-(7-bromo-2-oxo-1,5-naphthyridin-1(2H)-yl)ethyl)-2-oxabicyclo[2.2.2]octan-4-ylcarbamate (22.3 mg) was prepared from 7-bromo-1,5-naphthyridin-2(1H)-one (20.0 mg) and AD (15.0 mg) in the same manner as described for Step 1 of EXAMPLE 127.

¹H NMR (CDCl₃): δ 1.46 (s, 9H), 1.70-1.77 (m, 4H), 1.81-1.88 (m, 2H), 1.97-2.01 (m, 2H), 2.10-2.16 (m, 2H), 4.05 (s, 2H), 4.26-4.30 (m, 2H), 6.89 (d, J=9.8 Hz, 1H), 7.84 (d, J=9.8 Hz, 1H), 8.13 (d, J=1.2 Hz, 1H), 8.55 (d, J=1.8 Hz, 1H).

MS (ESI⁺) m/z: 478 (MH⁺).

HRMS (ESI⁺) for C₂₂H₂₉BrN₃O₄ (MH⁺): calcd, 478.13414. found, 478.13334.

### Step 2

### tert-Butyl 1-(2-(7-Cyano-2-oxo-1,5-naphthyridin-1(2H)-yl)ethyl)-2-oxabicyclo[2.2.2]octan-4-ylcarbamate

A mixture of tert-butyl 1-(2-(7-bromo-2-oxo-1,5-naphthyridin-1(2H)-yl)ethyl)-2-oxabicyclo[2.2.2]octan-4-ylcarbamate (200 mg) was prepared from 7-bromo-1,5-naphthyridin-2(1H)-one, zinc cyanide (50.0 mg) and tetrakis(triphenylphosphine)palladium (145 mg) in N-methyl-2-pyrrolidone (3.5 mL) was stirred at 80° C. for 7 hours, and then concentrated in vacuo. Flash chromatography (silica, hexane:ethyl acetate=1:1) of the residue gave tert-butyl 1-(2-(7-cyano-2-oxo-1,5-naphthyridin-1(2H)-yl)ethyl)-2-oxabicyclo[2.2.2]octan-4-ylcarbamate (179 mg).

¹H NMR (CDCl₃): δ 1.44 (s, 9H), 1.64-1.79 (m, 4H), 1.81-1.92 (m, 2H), 1.94-2.07 (m, 2H), 2.12-2.18 (m, 2H), 4.04 (s, 2H), 4.28-4.33 (m, 2H), 7.02 (d, J=9.8 Hz, 1H), 7.91 (d, J=9.8 Hz, 1H), 8.22 (s, 1H), 8.72 (d, J=1.8 Hz, 1H).

MS (ESI⁺) m/z: 425 (MH⁺).

HRMS (ESI⁺) for C₂₃H₂₉N₄O₄ (MH⁺): calcd, 425.21888. found, 425.21885.

### Step 3

### 5-(2-(4-Amino-2-oxabicyclo[2.2.2]octan-1-yl)ethyl)-6-oxo-5,6-dihydro-1,5-naphthyridine-3-carbonitrile

The title compound 5-(2-(4-amino-2-oxabicyclo[2.2.2]octan-1-yl)ethyl)-6-oxo-5,6-dihydro-1,5-naphthyridine-3-carbonitrile (60.3 mg) was prepared from tert-butyl 1-(2-(7-cyano-2-oxo-1,5-naphthyridin-1(2H)-yl)ethyl)-2-oxabicyclo[2.2.2]octan-4-ylcarbamate (100 mg) in the same manner as described for Step 2 of EXAMPLE 32.

¹H NMR (CDCl₃): δ 1.65-1.80 (m, 10H), 1.94-1.97 (m, 2H), 3.71 (s, 2H), 4.30-4.34 (m, 2H), 7.02 (d, J=9.8 Hz, 1H), 7.92 (d, J=9.8 Hz, 1H), 8.24 (s, 1H), 8.72 (d, J=1.8 Hz, 1H).

MS (ESI⁺) m/z: 325 (MH⁺).

HRMS (ESI⁺) for C₁₈H₂₁N₄O₂ (MH⁺): calcd, 325.16645. found, 325.16652.

### Step 4

### 6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridine-3-carbonitrile

The title compound 6-oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridine-3-carbonitrile (70.0 mg) was prepared from 5-(2-(4-amino-2-oxabicyclo[2.2.2]octan-1-yl)ethyl)-6-oxo-5,6-dihydro-1,5-naphthyridine-3-carbonitrile (58.0 mg) and I (39.0 mg) in the same manner as described for Step 3 of EXAMPLE 1.

¹H NMR (DMSO-d₆): δ 1.60-1.71 (m, 8H), 1.85-1.90 (m, 3H), 3.61 (s, 2H), 3.63 (s, 2H), 4.21-4.26 (m, 2H), 4.59 (s, 2H), 7.01 (d, J=7.9 Hz, 1H), 7.02 (d, J=9.8 Hz, 1H), 7.28 (d, J=7.9 Hz, 1H), 7.99 (d, J=9.8 Hz, 1H), 8.36 (s, 1H), 8.88 (d, J=1.8 Hz, 1H), 11.16 (br, 1H).

MS (ESI⁺) m/z: 487 (MH⁺).

HRMS (ESI⁺) for C₂₆H₂₇N₆O₄ (MH⁺): calcd, 487.20938. found, 487.20890.

### Example 129

### 6-((1-(2-(7-Chloro-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one

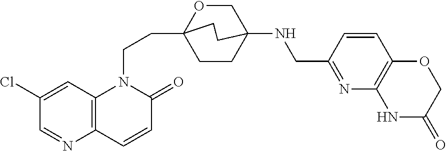 [SMILES: O=C1COc2ccc(CNC34CCC(CCn5c(=O)ccc6ncc(Cl)cc65)(CC3)OC4)nc2N1]

### Step 1

### tert-Butyl 1-(2-(7-Chloro-2-oxo-1,5-naphthyridin-1(2H)-yl)ethyl)-2-oxabicyclo[2.2.2]octan-4-ylcarbamate

The title compound tert-butyl 1-(2-(7-chloro-2-oxo-1,5-naphthyridin-1(2H)-yl)ethyl)-2-oxabicyclo[2.2.2]octan-4-ylcarbamate (109 mg) was prepared from 7-chloro-1,5-naphthyridin-2(1H)-one (100 mg) and AD (211 mg) in the same manner as described for Step 1 of EXAMPLE 127.

¹H NMR (CDCl₃): δ 1.44 (s, 9H), 1.69-1.79 (m, 4H), 1.85-1.95 (m, 2H), 2.00-2.07 (m, 2H), 2.13-2.19 (m, 2H), 4.04 (s, 2H), 4.24-4.36 (m, 2H), 6.87 (d, J=9.2 Hz, 1H), 7.86 (d, J=9.8 Hz, 1H), 7.96 (d, J=1.8 Hz, 1H), 8.46 (d, J=2.4 Hz, 1H).

MS (ESI⁺) m/z: 434 (MH⁺).

HRMS (ESI⁺) for C₂₂H₂₉ClN₃O₄ (MH⁺): calcd, 434.18466. found, 434.18483.

### Step 2

### 1-(2-(4-Amino-2-oxabicyclo[2.2.2]octan-1-yl)ethyl)-7-chloro-1,5-naphthyridin-2(1H)-one

The title compound 1-(2-(4-amino-2-oxabicyclo[2.2.2]octan-1-yl)ethyl)-7-chloro-1,5-naphthyridin-2(1H)-one (107 mg) was prepared from tert-butyl 1-(2-(7-chloro-2-oxo-1,5-naphthyridin-1(2H)-yl)ethyl)-2-oxabicyclo[2.2.2]octan-4-ylcarbamate (137 mg) in the same manner as described for Step 2 of EXAMPLE 32.

¹H NMR (CDCl₃): δ 1.62-1.80 (m, 8H), 1.80-2.05 (m, 2H), 3.69 (s, 2H), 4.25-4.33 (m, 2H), 6.88 (d, J=9.8 Hz, 1H), 7.86 (d, J=9.8 Hz, 1H), 7.95 (d, J=1.8 Hz, 1H), 8.46 (d, J=1.8 Hz, 1H).

MS (ESI⁺) m/z: 334 (MH⁺).

HRMS (ESI⁺) for C₁₇H₂₁ClN₃O₂ (MH⁺): calcd, 334.13223. found, 334.13196.

### Step 3

### 6-((1-(2-(7-Chloro-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one

The title compound 6-((1-(2-(7-chloro-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one (117 mg) was prepared from 1-(2-(4-amino-2-oxabicyclo[2.2.2]octan-1-yl)ethyl)-7-chloro-1,5-naphthyridin-2(1H)-one (94.0 mg) and I (53.0 mg) in the same manner as described for Step 3 of EXAMPLE 1.

¹H NMR (DMSO-d₆): δ 1.50-1.76 (m, 9H), 1.79-1.94 (m, 2H), 3.63 (s, 4H), 4.18-4.25 (m, 2H), 4.59 (s, 2H), 6.86 (d, J=9.8 Hz, 1H), 7.01 (d, J=8.6 Hz, 1H), 7.28 (d, J=8.6 Hz, 1H), 7.93 (d, J=9.8 Hz, 1H), 7.96 (d, J=1.8 Hz, 1H), 8.55 (d, J=1.8 Hz, 1H), 11.16 (br, 1H).

MS (ESI⁺) m/z: 496 (MH⁺).

HRMS (ESI⁺) for C₂₅H₂₇ClN₅O₄ (MH⁺): calcd, 496.17516. found, 496.17568.

### Example 130

### 6-((1-(2-(7-Bromo-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one

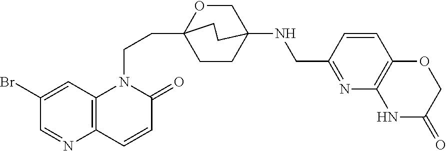 [SMILES: O=C1COc2ccc(CNC34CCC(CCn5c(=O)ccc6ncc(Br)cc65)(CC3)OC4)nc2N1]

### Step 1

### 1-(2-(4-Amino-2-oxabicyclo[2.2.2]octan-1-yl)ethyl)-7-bromo-1,5-naphthyridin-2(1H)-one

The title compound 1-(2-(4-amino-2-oxabicyclo[2.2.2]octan-1-yl)ethyl)-7-bromo-1,5-naphthyridin-2(1H)-one (76.2 mg) was prepared from tert-butyl 1-(2-(7-bromo-2-oxo-1,5-naphthyridin-1(2H)-yl)ethyl)-2-oxabicyclo[2.2.2]octan-4-ylcarbamate (120 mg) in the same manner as described for Step 2 of EXAMPLE 32.

¹H NMR (CDCl₃): δ 1.63-2.05 (m, 11H), 3.71 (s, 2H), 4.25-4.33 (m, 2H), 6.90 (d, J=9.7 Hz, 1H), 7.85 (d, J=9.7 Hz, 1H), 8.13 (d, J=1.8 Hz, 1H), 8.56 (d, J=1.8 Hz, 1H).

MS (ESI⁺) m/z: 378 (MH⁺).

HRMS (ESI⁺) for C₁₇H₂₁BrN₃O₂ (MH⁺): calcd, 378.08171. found, 378.08210.

### Step 2

### 6-((1-(2-(7-Bromo-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one

The title compound 6-((1-(2-(7-bromo-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one (43.8 mg) was prepared from 1-(2-(4-amino-2-oxabicyclo[2.2.2]octan-1-yl)ethyl)-7-bromo-1,5-naphthyridin-2(1H)-one (74.0 mg) and I (35.0 mg) in the same manner as described for Step 3 of EXAMPLE 1.

¹H NMR (DMSO-d₆): δ 1.57-1.91 (m, 11H), 3.63 (s, 4H), 4.19-4.23 (m, 2H), 4.59 (s, 2H), 6.88 (d, J=9.8 Hz, 1H), 7.01 (d, J=7.9 Hz, 1H), 7.28 (d, J=7.9 Hz, 1H), 7.91 (d, J=9.8 Hz, 1H), 8.11 (d, J=1.8 Hz, 1H), 8.62 (d, J=1.8 Hz, 1H), 11.16 (s, 1H).

MS (ESI⁺) m/z: 540 (MH⁺).

HRMS (ESI⁺) for C₂₅H₂₇BrN₅O₄ (MH⁺): calcd, 540.12464. found, 540.12498.

### Example 131

### 6-((1-(2-(7-Methoxy-2-oxo-1,8-naphthyridin-1(2H)-yl)ethyl)-2-oxabicyclo[2.2.2]octan-4-ylamino)methyl)-2H-pyrido[3,2-b][1,4]oxazin-3(4H)-one Hydrochloride

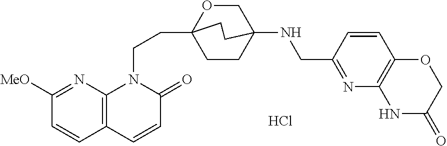 [SMILES: COc1ccc2ccc(=O)n(CCC34CCC(NCc5ccc6c(n5)NC(=O)CO6)(CC3)CO4)c2n1 ; Cl]

### Step 1

### tert-Butyl 1-(2-(7-Methoxy-2-oxo-1,8-naphthyridin-1(2H)-yl)ethyl)-2-oxabicyclo[2.2.2]octan-4-ylcarbamate

A suspension of 7-methoxy-1,8-naphthyridin-2(1H)-one (180 mg) and cesium carbonate (400 mg) in N,N-dimethylacetamide (3.4 mL) at room temperature for 1 hour. AD (390 mg) was added to the mixture. The resulting mixture was stirred at 60° C. for 2.5 hours and then concentrated in vacuo. After dilution of the residue with ethyl acetate, the mixture was washed with saturated ammonium chloride solution and brine, dried over anhydrous sodium sulfate, filtered, and then concentrated in vacuo. Flash chromatography (silica, hexane:ethyl acetate=1:1) of the residue gave tert-butyl 1-(2-(7-methoxy-2-oxo-1,8-naphthyridin-1(2H)-yl)ethyl)-2-oxabicyclo[2.2.2]octan-4-ylcarbamate (355 mg).

¹H NMR (CDCl₃): δ 1.43 (s, 9H), 1.73-1.90 (m, 6H), 1.99-2.15 (m, 4H), 3.93 (s, 2H), 4.04 (s, 3H), 4.28 (br, 1H), 4.51-4.58 (m, 2H), 6.56 (d, J=9.8 Hz, 1H), 6.59 (d, J=8.6 Hz, 1H), 7.54 (d, J=9.2 Hz, 1H), 7.70 (d, J=8.6 Hz, 1H).

MS (ESI⁺) m/z: 430 (MH⁺).

HRMS (ESI⁺) for C₂₃H₃₂N₃O₅ (MH⁺): calcd, 430.23420. found, 430.23423.

### Step 2

### 1-(2-(4-Amino-2-oxabicyclo[2.2.2]octan-1-yl)ethyl)-7-methoxy-1,8-naphthyridin-2(1H)-one

The title compound 1-(2-(4-amino-2-oxabicyclo[2.2.2]octan-1-yl)ethyl)-7-methoxy-1,8-naphthyridin-2(1H)-one (54.5 mg) was prepared from tert-butyl 1-(2-(7-methoxy-2-oxo-1,8-naphthyridin-1(2H)-yl)ethyl)-2-oxabicyclo[2.2.2]octan-4-ylcarbamate (70.0 mg) in the same manner as described for Step 2 of EXAMPLE 1.

¹H NMR (CDCl₃): δ 1.60-1.85 (m, 8H), 1.97-2.07 (m, 2H), 3.62 (s, 2H), 4.05 (s, 3H), 4.51-4.60 (m, 2H), 6.56 (d, J=9.2 Hz, 1H), 6.59 (d, J=8.6 Hz, 1H), 7.54 (d, J=9.2 Hz, 1H), 7.70 (d, J=7.9 Hz, 1H).

MS (ESI⁺) m/z: 330 (MH⁺).

HRMS (ESI⁺) for C₁₈H₂₄N₃O₃ (MH⁺): calcd, 330.18177. found, 330.18121.

### Step 3

### 6-((1-(2-(7-Methoxy-2-oxo-1,8-naphthyridin-1(2H)-yl)ethyl)-2-oxabicyclo[2.2.2]octan-4-ylamino)methyl)-2H-pyrido[3,2-b][1,4]oxazin-3(4H)-one

The title compound 6-((1-(2-(7-methoxy-2-oxo-1,8-naphthyridin-1(2H)-yl)ethyl)-2-oxabicyclo[2.2.2]octan-4-ylamino)methyl)-2H-pyrido[3,2-b][1,4]oxazin-3(4H)-one (46.6 mg) was prepared from 1-(2-(4-amino-2-oxabicyclo[2.2.2]octan-1-yl)ethyl)-7-methoxy-1,8-naphthyridin-2(1H)-one (40.0 mg) and I (22.7 mg) in the same manner as described for Step 3 of EXAMPLE 1.

¹H NMR (DMSO-d₆): δ 1.57-1.77 (m, 8H), 1.81-1.93 (m, 3H), 3.58 (s, 2H), 3.62 (m, 2H), 3.97 (s, 3H), 4.36-4.42 (m, 2H), 4.59 (s, 2H), 6.46 (d, J=9.7 Hz, 1H), 6.71 (d, J=7.9 Hz, 1H), 7.01 (d, J=7.9 Hz, 1H), 7.27 (d, J=7.9 Hz, 1H), 7.83 (d, J=9.1 Hz, 1H), 8.03 (d, J=8.5 Hz, 1H), 11.15 (s, 1H).

MS (ESI⁺) m/z: 492 (MH⁺).

HRMS (ESI⁺) for C₂₆H₃₀N₅O₅ (MH⁺): calcd, 492.22469. found, 492.22522.

### Step 4

### 6-((1-(2-(7-Methoxy-2-oxo-1,8-naphthyridin-1(2H)-yl)ethyl)-2-oxabicyclo[2.2.2]octan-4-ylamino)methyl)-2H-pyrido[3,2-b][1,4]oxazin-3(4H)-one Hydrochloride

The title compound 6-((1-(2-(7-methoxy-2-oxo-1,8-naphthyridin-1(2H)-yl)ethyl)-2-oxabicyclo[2.2.2]octan-4-ylamino)methyl)-2H-pyrido[3,2-b][1,4]oxazin-3(4H)-one hydrochloride (270 mg) was prepared from 6-((1-(2-(7-methoxy-2-oxo-1,8-naphthyridin-1(2H)-yl)ethyl)-2-oxabicyclo[2.2.2]octan-4-ylamino)methyl)-2H-pyrido[3,2-b][1,4]oxazin-3(4H)-one (280 mg) in the same manner as described for Step 4 of EXAMPLE 3.

¹H NMR (DMSO-d₆): δ 1.66-1.75 (m, 2H), 1.78-1.90 (m, 2H), 1.93-2.09 (m, 6H), 3.91 (s, 2H), 3.98 (s, 3H), 4.35-4.46 (m, 2H), 4.11 (m, 2H), 4.69 (m, 2H), 6.48 (d, J=9.7 Hz, 1H), 6.73 (d, J=8.5 Hz, 1H), 7.20 (d, J=7.9 Hz, 1H), 7.45 (d, J=8.5 Hz, 1H), 7.85 (d, J=9.7 Hz, 1H), 8.05 (d, J=8.5 Hz, 1H), 9.23 (br, 2H), 11.33 (s, 1H).

MS (ESI⁺) m/z: 492 (MH⁺) (as free base).

HRMS (ESI⁺) for C₂₆H₃₀N₅O₅ (MH⁺) (as free base): calcd, 492.22469. found, 492.22457.

The following examples EXAMPLE 132 EXAMPLE 135 were prepared from Enantiomer A of 1-(4-amino-2-oxabicyclo[2.2.2]octan-1-yl)-2-(3-fluoro-6-methoxy-1,5-naphthyridin-4-yl)ethanol and corresponding aldehydes in the same manner as described for Step 3 of EXAMPLE 1.

### Example 132

### 1-(4-((2,3-Dihydro-[1,4]dioxino[2,3-c]pyridin-7-yl)methylamino)-2-oxabicyclo[2.2.2]octan-1-yl)-2-(3-fluoro-6-metho xy-1,5-naphthyridin-4-yl)ethanol

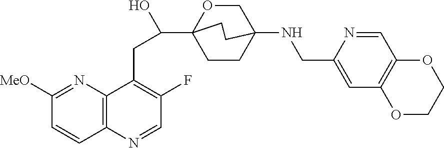 [SMILES: COc1ccc2ncc(F)c(CC(O)C34CCC(NCc5cc6c(cn5)OCCO6)(CC3)CO4)c2n1]

¹H NMR (DMSO-d₆): δ 1.56-2.02 (m, 9H), 2.97-3.06 (m, 1H), 3.31-3.41 (m, 1H), 3.55 (s, 2H), 3.61 (s, 2H), 3.71-3.77 (m, 1H), 4.02 (s, 3H), 4.23-4.28 (m, 2H), 4.29-4.35 (m, 2H), 4.44 (d, J=6.1 Hz, 1H), 6.92 (s, 1H), 7.20 (d, J=9.1 Hz, 1H), 7.98 (s, 1H), 8.25 (d, J=9.1 Hz, 1H), 8.72 (s, 1H).

MS (ESI⁺) m/z: 497 (MH⁺).

HRMS (ESI⁺) for C₂₆H₃₀FN₄O₅ (MH⁺): calcd, 497.22002. found, 497.21985.

### Example 133

### 3-((1-(2-(3-Fluoro-6-methoxy-1,5-naphthyridin-4-yl)-1-hydroxyethyl)-2-oxabicyclo[2.2.2]octan-4-ylamino)methyl)-1-methylquinolin-2(1H)-one

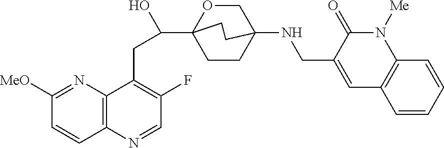 [SMILES: COc1ccc2ncc(F)c(CC(O)C34CCC(NCc5cc6ccccc6n(C)c5=O)(CC3)CO4)c2n1]

¹H NMR (DMSO-d₆): δ 1.60-2.05 (m, 9H), 2.99-3.08 (m, 1H), 3.26-3.35 (m, 1H), 3.58 (s, 2H), 3.62 (s, 2H), 3.64 (s, 3H), 3.71-3.80 (m, 1H), 4.03 (s, 3H), 4.45 (d, J=6.1 Hz, 1H), 7.21 (d, J=9.2 Hz, 1H), 7.26 (t, J=7.3 Hz, 1H), 7.51 (d, J=8.6 Hz, 1H), 7.57 (dd, J=8.6, 1.2 Hz, 1H), 7.71 (dd, J=7.9, 1.2 Hz, 1H), 7.88 (s, 1H), 8.26 (d, J=8.6 Hz, 1H), 8.72 (s, 1H).

MS (ESI⁺) m/z: 519 (MH⁺).

HRMS (ESI⁺) for C₂₉H₃₂FN₄O₆ (MH⁺): calcd, 519.24076. found, 519.24030.

### Example 134

### 7-((1-(2-(3-Fluoro-6-methoxy-1,5-naphthyridin-4-yl)-1-hydroxyethyl)-2-oxabicyclo[2.2.2]octan-4-ylamino)methyl)-1H-pyrido[3,4-b][1,4]oxazin-2(3H)-one

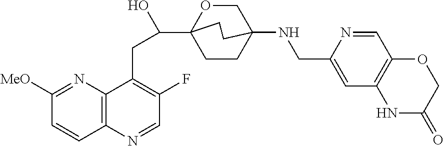 [SMILES: COc1ccc2ncc(F)c(CC(O)C34CCC(NCc5cc6c(cn5)OCC(=O)N6)(CC3)CO4)c2n1]

¹H NMR (DMSO-d₆): δ 1.57-1.88 (m, 7H), 1.93-2.03 (m, 1H), 3.02 (dd, J=12.2, 10.4 Hz, 1H), 3.32-3.38 (m, 1H), 3.57 (s, 2H), 3.64 (s, 2H), 3.71-3.78 (m, 1H), 4.02 (s, 3H), 4.44 (d, J=6.1 Hz, 1H), 4.63 (s, 2H), 6.97 (s, 1H), 7.21 (d, J=9.2 Hz, 1H), 8.03 (s, 1H), 8.26 (d, J=9.2 Hz, 1H), 8.72 (s, 1H), 10.99 (brs, 1H).

MS (ESI⁺) m/z: 510 (MH⁺).

HRMS (ESI⁺) for C₂₆H₂₉FN₅O₅ (MH⁺): calcd, 510.21527. found, 510.21498.

### Example 135

### 7-Fluoro-6-((1-(2-(3-fluoro-6-methoxy-1,5-naphthyridin-4-yl)-1-hydroxyethyl)-2-oxabicyclo[2.2.2]octan-4-ylamino)methyl)-2H-pyrido[3,2-b][1,4]oxazin-3(4H)-one

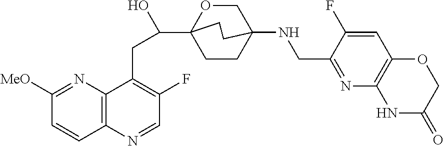 [SMILES: COc1ccc2ncc(F)c(CC(O)C34CCC(NCc5nc6c(cc5F)OCC(=O)N6)(CC3)CO4)c2n1]

¹H NMR (DMSO-d₆): δ 1.63-1.84 (m, 8H), 1.95-2.01 (m, 1H), 3.03 (t, J=10.4 Hz, 1H), 3.36 (br, 1H), 3.55 (s, 2H), 3.65-3.77 (m, 3H), 4.02 (s, 3H), 4.46 (d, J=6.1 Hz, 1H), 4.64 (s, 2H), 7.21 (d, J=9.2 Hz, 1H), 7.41 (d, J=9.8 Hz, 1H), 8.26 (d, J=9.2 Hz, 1H), 8.73 (s, 1H), 11.29 (br, 1H).

MS (ESI⁺) m/z: 528 (MH⁺).

HRMS (ESI) for C₂₆H₂₈F₂N₅O₅ (MH⁺): calcd, 528.20585. found, 528.20592.

### Example 135

### 6-({1-[(3-Fluoro-6-methoxy-[1,5]naphthyridin-4-ylamino)-methyl]-2-oxa-bicyclo[2.2.2]oct-4-ylamino}-methyl)-4H-pyrido[3,2-b][1,4]oxazin-3-one

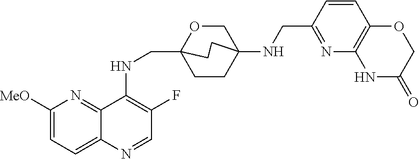 [SMILES: COc1ccc2ncc(F)c(NCC34CCC(NCc5ccc6c(n5)NC(=O)CO6)(CC3)CO4)c2n1]

### Step 1

### tert-Butyl {1-[(3-Fluoro-6-methoxy-[1,5]naphthyridin-4-ylamino)-methyl]-2-oxa-bicyclo[2.2.2]oct-4-yl}carbamate

To a solution of AI (80 mg) in 1,4-dioxane (5 mL) was added L (80 mg), cesium carbonate (146.6 mg), Pd₂(dba)₃ (10 mg) and Xantphos (Sigma-Aldrich, St. Louis, Mo.) (10 mg). The mixture was stirring overnight at 100° C. under N₂. The residue was diluted with ethyl acetate and washed with water and brine, dried and condensed. The residue was purified by prep-TLC and gave the title compound. MS m/z: 433 (MH⁺).

### Step 2

### N-[(4-Amino-2-oxabicyclo[2.2.2]oct-1-yl)methyl]-3-fluoro-6-methoxy-1,5-naphthyridin-4-amine

To a solution of tert-butyl {1-[(3-fluoro-6-methoxy-[1,5]naphthyridin-4-ylamino)-methyl]-2-oxa-bicyclo[2.2.2]oct-4-yl}carbamate (40 mg) in dichloromethane (2 mL) was added trifluoroacetic acid (2 mL) and the mixture was stirred at room temperature for 30 minutes and concentrated in vacuo. After dilution of the residue with water, the mixture was washed with methyl tert-butyl ether twice. The aqueous layer was adjusted to pH 13 by addition of aqueous sodium carbonate solution and extracted twice with ethyl acetate. The combined ethyl acetate layer was washed with brine, dried over anhydrous sodium sulfate and condensed to give the pure title compound. MS m/z: 333 (MH⁺).

### Step 3

A mixture of N-[(4-amino-2-oxabicyclo[2.2.2]oct-1-yl)methyl]-3-fluoro-6-methoxy-1,5-naphthyridin-4-amine (30 mg crude) and I (24 mg) in anhydrous N,N-dimethylformamide (3.5 mL) was added acetic acid (0.5 mL) was stirred at room temperature for 30 minutes. The resulting solution was added three times of sodium triacetoxyborohydride (38.4 mg) and stirred at room temperature for overnight. The mixture was concentrated in vacuo. After dilution of the residue with dichloromethane, the mixture was washed with saturated sodium carbonate solution, water and brine. The organic extracts were dried over anhydrous sodium sulfate then concentrated in vacuo. The residue was purified by prep-TLC (dichloromethane:methanol=10:1) to give a solid (15 mg). To a solution of this solid (15 mg) in dichloromethane (2 mL) and ethanol (0.5 mL) was added a solution of hydrogen chloride (7.5 uL, 4 M in 1,4-dioxane) under cooling with ice, the mixture was stirred at room temperature for 2 hours and concentrated in vacuo. Treatment of the residue with ethanol gave title compound.

¹H NMR (MeOD): δ 1.89-1.91 (m, 2H), 2.06-2.21 (m, 6H), 3.85 (s, 2H), 3.95 (s, 2H), 4.07 (s, 3H), 4.12 (s, 2H), 4.59 (s, 2H), 7.02 (d, J=7.6 Hz, 1H), 7.25 (d, J=7.6 Hz, 1H), 7.35 (d, J=8.8 Hz, 1H), 8.12 (d, J=8.8 Hz, 1H), 8.60 (d, J=7.6 Hz, 1H).

MS m/z: 495 (MH⁺).

### Example 137

### 6-[({1-[2-(6-Ethoxy-3-fluoro-8-methyl-1,5-naphthyridin-4-yl)ethyl]-2-oxabicyclo[2.2.2]oct-4-yl}amino)methyl]-2H-pyrido[3,2-b][1,4]oxazin-3(4H)-one

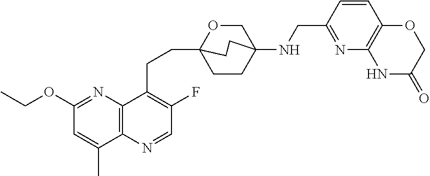 [SMILES: CCOc1cc(C)c2ncc(F)c(CCC34CCC(NCc5ccc6c(n5)NC(=O)CO6)(CC3)CO4)c2n1]

### Step 1

To a solution of B (100 mg) in anhydrous tetrahydrofuran (1.8 mL) was added a solution of 9-borabicyclo[3.3.1]nonane dimer (1.6 mL, 0.5 M in tetrahydrofuran) under cooling with ice, the mixture was stirred at room temperature for 1 hour. After quenching the reaction by adding water (1 drop) under cooling, the mixture was added AJ (120 mg), tetrakis(triphenylphosphine)palladium (100 mg), tripotassium phosphate (0.6 g) and ethanol/water (2 mL, 4:1), and degassed. The mixture was heated at 70° C. for 12 hours and concentrated in vacuo. After dilution of the residue with ethyl acetate, the mixture was washed with water and brine, dried over anhydrous sodium sulfate, filtered, and then concentrated in vacuo. The two products, tert-butyl (1-{2-[3-fluoro-8-methyl-6-(methylsulfonyl)-1,5-naphthyridin-4-yl]ethyl}-2-oxabicyclo[2.2.2]oct-4-yl)carbamate and tert-butyl {1-[2-(6-ethoxy-3-fluoro-8-methyl-1,5-naphthyridin-4-yl)ethyl]-2-oxabicyclo[2.2.2]oct-4-yl}carbamate were separated from each other. MS m/z: 460 (MH⁺).

### Step 2

### 1-(2-(6-Ethoxy-3-fluoro-8-methyl-1,5-naphthyridin-4-yl)ethyl)-2-oxabicyclo[2.2.2]octan-4-amine

To a solution of tert-butyl {1-[2-(6-ethoxy-3-fluoro-8-methyl-1,5-naphthyridin-4-yl)ethyl]-2-oxabicyclo[2.2.2]oct-4-yl}carbamate (120 mg crude) in dichloromethane (2 mL) was added trifluoroacetic acid (2 mL) and the mixture was stirred at room temperature for 30 minutes and concentrated in vacuo. After dilution of the residue with water, the mixture was washed with methyl tert-butyl ether twice. The aqueous layer was adjusted to pH 13 by addition of aqueous sodium carbonate solution and extract twice with ethyl acetate. The combined ethyl acetate layer was washed with brine, dried over anhydrous sodium sulfate and condensed to give crude the title compound. MS m/z: 360 (MH⁺).

### Step 3

### 6-((1-(2-(6-Ethoxy-3-fluoro-8-methyl-1,5-naphthyridin-4-yl)ethyl)-2-oxabicyclo[2.2.2]octan-4-ylamino)methyl)-2H-pyrido[3,2-b][1,4]oxazin-3(4H)-one

A mixture of 1-(2-(6-ethoxy-3-fluoro-8-methyl-1,5-naphthyridin-4-yl)ethyl)-2-oxabicyclo[2.2.2]octan-4-amine (80 mg crude) and I (110 mg) in anhydrous N,N-dimethylformamide (3.5 mL) was added acetic acid (0.5 mL) was stirred at room temperature for 30 minutes. The resulting solution was added three times of sodium triacetoxyborohydride (160 mg) and stirred at room temperature for overnight. The mixture was concentrated in vacuo. After dilution of the residue with dichloromethane, the mixture was washed with saturated sodium carbonate solution, water and brine. The organic extracts were dried over anhydrous sodium sulfate then concentrated in vacuo. The residue was purified by prep-TLC (dichloromethane:methanol=10:1) to afford a solid. To a solution this solid (45 mg) in dichloromethane (2 mL) and ethanol (0.5 mL) was added a solution of hydrogen chloride (21 uL, 4 M in 1,4-dioxane) under cooling with ice, the mixture was stirred at room temperature for 2 hours and concentrated in vacuo. Treatment of the residue with ethanol gave the title product.

¹H NMR (MeOD): δ 1.47 (t, J=7.2 Hz, 3H), 1.74-1.81 (m, 2H), 1.89-2.01 (m, 2H), 2.09-2.21 (m, 6H), 2.69 (s, 3H), 3.23 (t, J=8.0 Hz, 2H), 4.00 (s, 2H), 4.21 (s, 2H), 4.55 (q, J=7.2 Hz, 2H), 4.72 (s, 2H), 7.00 (s, 1H), 7.12 (d, J=8.0 Hz, 1H), 7.39 (d, J=8.0 Hz, 1H), 8.59 (s, 1H).

MS m/z: 524 (MH⁺).

### Example 138

### 6-[({1-[1,1,1-Trifluoro-3-(3-fluoro-6-methoxy-1,5-naphthyridin-4-yl)-2-hydroxypropan-2-yl]-2-oxabicyclo[2.2.2]oct-4-yl}amino)methyl]-2H-pyrido[3,2-b][1,4]oxazin-3(4H)-one

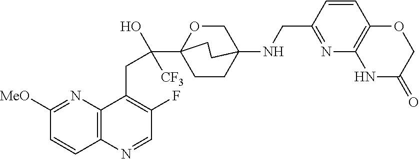 [SMILES: COc1ccc2ncc(F)c(CC(O)(C(F)(F)F)C34CCC(NCc5ccc6c(n5)NC(=O)CO6)(CC3)CO4)c2n1]

### Step 1

### tert-Butyl 1-(2,2,2-Trifluoro-1-hydroxyethyl)-2-oxabicyclo[2.2.2]octan-4-ylcarbamate

A solution of F (762 mg) and trimethyl(trifluoromethyl)silane (1.14 g) in N,N-dimethylformamide (20 mL) was cooled to 0° C. with ice-water. To this solution was added powdered cesium fluoride (1.3 g) in small batches. The mixture was stirred overnight at room temperature, diluted with ethyl acetate (50 mL), washed with water and brine, condensed. The residue was purified by column chromatography (25% ethyl acetate in petroleum ether) to give the title compound (230 mg). MS m/z: 326 (MH⁺).

### Step 2

### tert-Butyl 1-(2,2,2-Trifluoroacetyl)-2-oxabicyclo[2.2.2]octan-4-ylcarbamate

A suspension of tert-butyl 1-(2,2,2-trifluoro-1-hydroxyethyl)-2-oxabicyclo[2.2.2]octan-4-ylcarbamate (230 mg) and Dess-Martin periodinane (452 mg) was stirred overnight at room temperature. Filtered and the solid was washed with dichloromethane. The filtrate was condensed and the residue was purified by prep-TLC (petroleum ether:ethyl acetate=3:1) to afford a white solid (160 mg).

¹H NMR (CDCl₃): δ 1.39 (s, 9H), 1.76-1.83 (m, 2H), 1.84-1.92 (m, 2H), 1.95-2.21 (m, 6H), 4.00 (s, 2H).

### Step 3

### tert-Butyl 1-(1,1,1-Trifluoro-3-(3-fluoro-6-methoxy-1,5-naphthyridin-4-yl)-2-hydroxypropan-2-yl)-2-oxabicyclo[2.2.2]octan-4-ylcarbamate

A solution of R (192 mg) in tetrahydrofuran (4 mL) was added lithium diisopropyl amide (0.5 mL, 2.0 M in tetrahydrofuran) dropwise at −78° C. and stirred for 15 minutes. To this mixture was added dropwise a solution of tert-butyl 1-(2,2,2-trifluoroacetyl)-2-oxabicyclo[2.2.2]octan-4-ylcarbamate (160 mg) in tetrahydrofuran (1 mL). The resulting mixture was stirred at −78° C. for 30 minutes then warmed to room temperature and stirred overnight. Quenched the reaction by adding saturated ammonium chloride solution and extracted with ethyl acetate twice. The organic layer was condensed and the residue was purified by prep-TLC (petroleum ether:ethyl acetate=3:1) to afford a white solid (37 mg). MS m/z: 516 (MH⁺)

### Step 4

### 2-(4-Amino-2-oxabicyclo[2.2.2]octan-1-yl)-1,1,1-trifluoro-3-(3-fluoro-6-methoxy-1,5-naphthyridin-4-yl)propan-2-ol

To a solution of tert-butyl 1-(1,1,1-trifluoro-3-(3-fluoro-6-methoxy-1,5-naphthyridin-4-yl)-2-hydroxypropan-2-yl)-2-oxabicyclo[2.2.2]octan-4-ylcarbamate (37 mg) in dichloromethane (1 mL) was added trifluoroacetic acid (1 mL) and the mixture was stirred at room temperature for 30 minutes and concentrated in vacuo. After dilution of the residue with water, the mixture was washed with methyl tert-butyl ether twice. The aqueous layer was adjusted to pH 13 by addition of aqueous sodium carbonate solution and extract twice with ethyl acetate. The combined ethyl acetate layer was washed with brine, dried over anhydrous sodium sulfate and condensed to give the title compound (20 mg). MS m/z: 416 (MH⁺).

### Step 5

### 6-((1-(1,1,1-Trifluoro-3-(3-fluoro-6-methoxy-1,5-naphthyridin-4-yl)-2-hydroxypropan-2-yl)-2-oxabicyclo[2.2.2]octan-4-ylamino)methyl)-2H-pyrido[3,2-b][1,4]oxazin-3(4H)-one

A mixture of 2-(4-amino-2-oxabicyclo[2.2.2]octan-1-yl)-1,1,1-trifluoro-3-(3-fluoro-6-methoxy-1,5-naphthyridin-4-yl)propan-2-ol (20 mg) and I (13 mg) in anhydrous N,N-dimethylformamide (3.5 mL) was added acetic acid (0.5 mL) was stirred at room temperature for 30 minutes. The resulting solution was added three times of sodium triacetoxyborohydride (21 mg) and stirred at room temperature for overnight. The mixture was concentrated in vacuo. After dilution of the residue with dichloromethane, the mixture was washed with saturated sodium carbonate solution, water and brine. The organic extracts were dried over anhydrous sodium sulfate then concentrated in vacuo. The residue was purified by prep-TLC (dichloromethane:methanol=10:1) to give a solid. To a solution of this solid (17 mg) in dichloromethane (2 mL) and ethanol (0.5 mL) was added a solution of hydrogen chloride (7.3 uL, 4 M in dioxane) under cooling with ice, the mixture was stirred at room temperature for 2 hours and concentrated in vacuo. Treatment of the residue with ethanol gave the title compound.

¹H NMR (MeOD): δ 2.05-2.16 (m, 6H), 2.42-2.54 (m, 2H), 3.74 (d, J=14.4 Hz, 1H), 3.85 (d, J=14.8 Hz, 1H), 3.95-4.01 (m, 2H), 4.12 (s, 3H), 4.21 (s, 2H), 4.68 (s, 2H), 7.10 (d, J=7.6 Hz, 1H), 7.27 (d, J=9.2 Hz, 1H), 7.34 (d, J=7.6 Hz, 1H), 8.30 (d, J=9.2 Hz, 1H), 8.77 (s, 1H).

MS m/z: 578 (MH⁺).

### Example 139

### 6-((1-(2-(3-Fluoro-6-methoxy-8-methylquinolin-4-yl)ethyl)-2-oxabicyclo[2.2.2]octan-4-ylamino)methyl)-2H-pyrido[3,2-b][1,4]oxazin-3(4H)-one

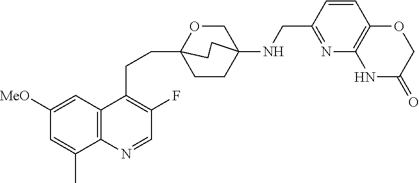 [SMILES: COc1cc(C)c2ncc(F)c(CCC34CCC(NCc5ccc6c(n5)NC(=O)CO6)(CC3)CO4)c2c1]

### Step 1

### 4-Methoxy-2-methylaniline

A solution of 4-methoxy-2-methyl-1-nitrobenzene (20.0 g) in methanol (150 mL) was added Pd/C (1.0 g), then stirred under H₂ for about 15 hours until the starting material disappeared on TLC. Filtered and the filtrate was concentrated under reduced pressure to give the title compound (16.5 g), which was used for the next step directly.

### Step 2

### Diethyl 2-((4-Methoxy-2-methylphenylamino)methylene)malonate

A solution of 4-methoxy-2-methylaniline (10.4 g) and diethyl ethoxymethylenemalonate (16.4 g) in toluene (60 mL) was stirred under reflux for 5 hours, concentrated under reduced pressure and recrystallized from petroleum ether to give the title compound (14.4 g). MS m/z: 308 (MH⁺).

### Step 3

### Ethyl 4-Hydroxy-6-methoxy-8-methylquinoline-3-carboxylate

A suspension of diethyl 2-((4-methoxy-2-methylphenylamino)methylene) malonate (8.0 g) in diphenyl ether (35 mL) was stirred under reflux for about 15 minutes until diethyl 2-((4-methoxy-2-methylphenylamino)methylene)malonate disappeared on TLC. Cooled to about 60° C., petroleum ether was added to give the title compound as a solid (5.0 g). MS m/z: 262 (MH⁺).

### Step 4

### Ethyl 4-Bromo-6-methoxy-8-methylquinoline-3-carboxylate

At 0° C., to a solution of ethyl 4-hydroxy-6-methoxy-8-methylquinoline-3-carboxylate (5.0 g) in N,N-dimethylformamide (35 mL) was added phosphorous tribromide (7.8 g) dropwise and then kept stirred at room temperature for 15 hours. Treated by saturated sodium carbonate solution until pH 8-9, extracted with ethyl acetate, the organic layers were washed by brine, dried over sodium sulfate and concentrated. The residue was recrystallized from petroleum ether to give the title compound (2.7 g). MS m/z: 324 (MH⁺).

### Step 5

### 4-Bromo-6-methoxy-8-methylquinoline-3-carboxylic Acid

A solution of ethyl 4-bromo-6-methoxy-8-methylquinoline-3-carboxylate (2.2 g) in tetrahydrofuran (30 mL) and 2 N sodium hydroxide solution (30 mL) was stirred at 60° C. for 5 hours. The mixture was acidified by diluted hydrochloric acid until pH 4-5, and the solid was filtered and dried to give the title compound (1.2 g).

¹H NMR (DMSO-d₆): δ 2.67 (s, 3H), 3.91 (s, 3H), 7.40 (s, 1H), 7.44 (s, 1H), 8.83 (s, 1H).

### Step 6

### tert-Butyl 4-Bromo-6-methoxy-8-methylquinolin-3-ylcarbamate

A suspension of 4-bromo-6-methoxy-8-methylquinoline-3-carboxylic acid (1.2 g) in 1,2-dichloroethane (15 mL) was added N-methylmorpholine (1.24 g) dropwise until a clear solution was obtained, then diphenyl phosphoryl azide (1.38 g) was added dropwise, kept stirred at room temperature for one hour and then under reflux for 2 hours until the starting material disappeared on TLC. Then tert-butanol was added and stirred under reflux overnight. The mixture was partitioned between water and dichloromethane. The organic layers were washed with brine, dried over sodium sulfate and concentrated. The residue was purified via flash-chromatography to give the title compound (0.5 g). MS m/z: 367 (MH⁺).

### Step 7

### 4-Bromo-6-methoxy-8-methylquinolin-3-amine

A solution of tert-butyl 4-bromo-6-methoxy-8-methylquinolin-3-ylcarbamate

(0.5 g) in dichloromethane (10 mL) was added trifluoroacetic acid (5 mL) and the mixture was stirred at room temperature for one hour. Concentrated, the residue was purified by flash chromatography to give the title compound (273 mg), which was used for the next step directly.

### Step 8

### 4-Bromo-3-fluoro-6-methoxy-8-methylquinoline

To a solution of 4-bromo-6-methoxy-8-methylquinolin-3-amine (266 mg) in tetrahydrofuran (3 mL) was added nitrosyl tetrafluoroborate (140 mg) at −10° C. under N₂. And the mixture was stirred at the same temperature for 1 hour. The solid was filtered and suspended into decahydro-naphthalene (3 mL), stirred at 120° C. for 15 minutes. Then the mixture was pass through a simple flash to remove decahydro-naphthalene and then washed by dichloromethane to give crude product, which was purified by prep-HPLC to give pure title compound (71 mg). MS m/z: 270 (MH⁺).

### Step 9

### tert-Butyl 1-(2-(3-Fluoro-6-methoxy-8-methylquinolin-4-yl)ethyl)-2-oxabicyclo[2.2.2]octan-4-ylcarbamate

At 0° C., under the protection of nitrogen, to a solution of compound B (80 mg) in dried tetrahydrofuran (2 mL) was added 9-borabicyclo(3.3.1)nonane dimer (1.0 mL) dropwise and the mixture was kept stirred at room temperature for 2 hours. Then 5 drops of water was added and kept stirred at room temperature for about 10 minutes. Then 4-bromo-3-fluoro-6-methoxy-8-methylquinoline (56 mg), tripotassium phosphate (400 mg), lithium chloride (200 mg) and tetrakis(triphenylphosphine)palladium (80 mg) was added to the mixture, and then ethanol (2 mL) and water (0.5 mL) was added. The resulting mixture was stirred under nitrogen at reflux for about 1 hour, partitioned between water and ethyl acetate. The organic layers were washed by brine, dried over sodium sulfate, concentrated to give the crude title compound (67 mg), which was used for the next step directly.

### Step 10

### 1-(2-(3,8-Difluoro-6-methoxyquinolin-4-yl)ethyl)-2-oxabicyclo[2.2.2]octan-4-amine

A solution of tert-butyl 1-(2-(3-fluoro-6-methoxy-8-methylquinolin-4-yl)ethyl)-2-oxabicyclo[2.2.2]octan-4-ylcarbamate (67 mg) in dichloromethane (5 mL) was added trifluoroacetic acid (5 mL) kept stirred at room temperature for 1 hour and then concentrated, and the residue was partitioned between water and methyl tert-butyl ether. The aqueous layer was basified by sodium carbonate until pH 8-9, and extracted by ethyl acetate. The organic layers were washed by brine, dried over sodium sulfate, concentrated to give the title compound (33 mg). MS m/z: 345 (MH⁺).

### Step 11

### 6-((1-(2-(3-Fluoro-6-methoxy-8-methylquinolin-4-yl)ethyl)-2-oxabicyclo[2.2.2]octan-4-ylamino)methyl)-2H-pyrido[3,2-b][1,4]oxazin-3(4H)-one

A solution of 1-(2-(3,8-difluoro-6-methoxyquinolin-4-yl)ethyl)-2-oxabicyclo[2.2.2]octan-4-amine (33 mg) and I (27 mg) in N,N-dimethylformamide:acetic acid=7:1 (5 mL) was stirred at room temperature for 30 minutes and then sodium triacetoxyborohydride (67 mg) was added. The mixture was and stirred at room temperature for overnight and the mixture was purified by prep-HPLC to give the title compound (15 mg).

¹H NMR (MeOD): δ 1.73-1.96 (m, 4H), 2.06-2.18 (m, 6H), 2.68 (s, 3H), 3.10-3.16 (m, 2H), 3.93 (s, 3H), 4.05 (s, 2H), 4.22 (s, 2H), 4.69 (s, 2H), 7.08-7.11 (d, J=8.6 Hz, 1H), 7.23 (s, 2H), 7.35-7.37 (d, J=8.6 Hz, 1H), 8.54 (s, 1H).

MS m/z: 510 (MH⁺).

### Example 140

### 6-[({1-[2-(3-Fluoro-6-methoxy-8-methylquinolin-4-yl)-1-hydroxyethyl]-2-oxabicyclo[2.2.2]oct-4-yl}amino)methyl]-2H-pyrido[3,2-b][1,4]oxazin-3(4H)-one

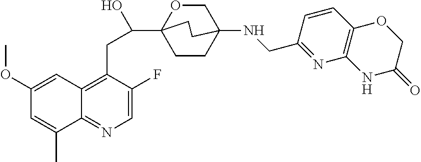 [SMILES: COc1cc(C)c2ncc(F)c(CC(O)C34CCC(NCc5ccc6c(n5)NC(=O)CO6)(CC3)CO4)c2c1]

### Step 1

### tert-Butyl 1-(1-Hydroxyethyl)-2-oxabicyclo[2.2.2]octan-4-ylcarbamate

At −78° C., to a solution of F (770 mg) in dried tetrahydrofuran (30 mL) was added a solution of methylmagnesium bromide (2.5 mL, 3 M) dropwise, and then warmed to room temperature slowly. The mixture was treated by saturated ammonium chloride solution and extracted with ethyl acetate. The organic layers were washed by brine, dried over sodium sulfate, concentrated, and purified by flash chromatography to give the title compound (392 mg). MS m/z: 272 (MH⁺).

### Step 2

### tert-Butyl 1-Acetyl-2-oxabicyclo[2.2.2]octan-4-ylcarbamate

To a solution of tert-butyl 1-(1-hydroxyethyl)-2-oxabicyclo[2.2.2]octan-4-ylcarbamate (392 mg) in dried dichloromethane (15 mL) was added Dess-Martin periodinane (3.0 g), the resulting mixture was kept stirred at room temperature for 24 hours. Filtered, and the filtrate was concentrated and purified by flash chromatography to give the title compound (280 mg).

¹H NMR (MeOD): δ 1.39 (s, 9H), 1.84-2.06 (m, 8H), 2.14 (s, 3H), 3.97 (s, 2H).

### Step 3

### tert-Butyl 1-(1-(Trimethylsilyloxy)vinyl)-2-oxabicyclo[2.2.2]octan-4-ylcarbamate

At −78° C., to a solution of tert-butyl 1-acetyl-2-oxabicyclo[2.2.2]octan-4-ylcarbamate (200 mg) in dried tetrahydrofuran (8 mL) was added lithium bis(trimethylsilyl)amide (1.7 mL) dropwise and then kept stirred at the temperature for half an hour. Then chlorotrimethylsilane (96 mg) was added and stirred at the temperature for another half an hour. The mixture was brought to 0° C. slowly and treated by saturated ammonium chloride solution, extracted by ethyl acetate. The organic layers were washed by brine, dried over sodium sulfate, concentrated to give the title compound (198 mg), which was used for the next step directly.

### Step 4

### tert-Butyl 1-(2-(3-Fluoro-6-methoxy-8-methylquinolin-4-yl)acetyl)-2-oxabicyclo[2.2.2]octan-4-ylcarbamate

A suspension of tert-butyl 1-(1-(trimethylsilyloxy)vinyl)-2-oxabicyclo[2.2.2]octan-4-ylcarbamate (100 mg), 4-bromo-3-fluoro-6-methoxy-8-methylquinoline (54 mg), Pd₂(dba)₃ (20 mg), s-Phos (Sigma-Aldrich, St. Louis, Mo.) (20 mg) and zinc fluoride (36 mg) in N,N-dimethylformamide (3 mL) was kept at a microwave condition at 150° C. for 15 minutes. The mixture was partitioned between water and ethyl acetate. The organic layers were washed by brine, dried over sodium sulfate, concentrated to give the crude title compound (78 mg), which was used for the next step directly.

### Step 5

### 1-(4-Amino-2-oxabicyclo[2.2.2]octan-1-yl)-2-(3-fluoro-6-methoxy-8-methylquinolin-4-yl)ethanone

A solution of tert-butyl 1-(2-(3-fluoro-6-methoxy-8-methylquinolin-4-yl)acetyl)-2-oxabicyclo[2.2.2]octan-4-ylcarbamate (78 mg) in dichloromethane (6 mL) was added trifluoroacetic acid (6 mL) kept stirred at room temperature for approximately 1 hour and then concentrated. The residue was partitioned between water and methyl tert-butyl ether, and the aqueous phase was basified by sodium carbonate until pH 8-9. Extracted with ethyl acetate, the organic layers were washed by brine, dried over sodium sulfate, concentrated to give the title compound (35 mg). MS m/z: 359 (MH⁺).

### Step 6

### 1-(4-Amino-2-oxabicyclo[2.2.2]octan-1-yl)-2-(3-fluoro-6-methoxy-8-methylquinolin-4-yl)ethanol

At 0° C., to a solution of 1-(4-amino-2-oxabicyclo[2.2.2]octan-1-yl)-2-(3-fluoro-6-methoxy-8-methylquinolin-4-yl)ethanone (35 mg) in methanol (10 mL) was added sodium borohydride (15 mg). The mixture was kept stirred at room temperature for 30 minutes, and drops of water were added and the resulting mixture was concentrated under reduced pressure. The residue was partitioned between water and ethyl acetate, the organic layers were gathered, washed by brine, dried over sodium sulfate, concentrated to give the crude title compound (36 mg). MS m/z: 361 (MH⁺).

### Step 7

### 6-((1-(2-(3-Fluoro-6-methoxy-8-methylquinolin-4-yl)-1-hydroxyethyl)-2-oxabicyclo[2.2.2]octan-4-ylamino)methyl)-2H-pyrido[3,2-b][1,4]oxazin-3(4H)-one

A solution of compound 1-(4-amino-2-oxabicyclo[2.2.2]octan-1-yl)-2-(3-fluoro-6-methoxy-8-methylquinolin-4-yl)ethanol (36 mg) and I (35 mg) in N,N-dimethylformamide:acetic acid=7:1 (5 mL) was stirred at room temperature for 30 minutes and then sodium triacetoxyborohydride (63 mg) was added. The mixture was stirred at room temperature for another 5 hours, and then purified by prep-HPLC to give the title compound (9 mg).

¹H NMR (MeOD): δ 1.86-2.06 (m, 8H), 2.67 (s, 3H), 3.06-3.12 (m, 1H), 3.50-3.64 (m, 2H), 3.84-3.90 (m, 7H), 4.63 (s, 2H), 6.99-7.01 (d, J=7.8 Hz, 1H), 7.17 (s, 1H), 7.28-7.30 (m, 2H), 8.51 (s, 1H).

MS m/z: 523 (MH⁺).

### Example 141

### 6-[({1-[2-(3,7-Difluoro-6-methoxyquinolin-4-yl)ethyl]-2-oxabicyclo[2.2.2]oct-4-yl}amino)methyl]-2H-pyrido[3,2-b][1,4]oxazin-3(4H)-one

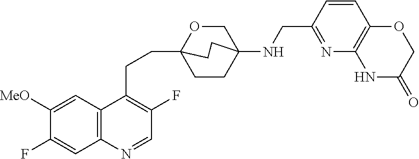 [SMILES: COc1cc2c(CCC34CCC(NCc5ccc6c(n5)NC(=O)CO6)(CC3)CO4)c(F)cnc2cc1F]

### Step 1

### Ethyl 7-Fluoro-4-hydroxy-6-methoxyquinoline-3-carboxylate

A mixture of 3-fluoro-4-methoxyaniline (1.4 g) and diethyl ethoxymethylenemalonate (2.2 g) in toluene (80 mL) was refluxed for 1 hour, condensed to dryness to afford a solid and added portionwise to diphenyl ether (10 mL) at 260° C. and refluxed for 8 minutes. The mixture was cooled to 60° C. and diluted with petroleum ether. The resulting precipitates were collected by filtrate and washed with petroleum ether to give the crude title compound (1.1 g). MS m/z: 266 (MH⁺).

### Step 2

### Ethyl 4-Bromo-7-fluoro-6-methoxyquinoline-3-carboxylate

To a suspension of ethyl 7-fluoro-4-hydroxy-6-methoxyquinoline-3-carboxylate (1.1 g, crude) in N,N-dimethylformamide (20 mL) was added phosphorous tribromide (1.3 g) under cooling with water. The mixture was stirred at room temperature for 30 minutes then poured into ice water, the mixture was adjusted to pH 10 by addition of saturated sodium hydrogencarbonate solution. The resulting precipitates were collected by filtrate and washed with water. The wet cake (0.5 g) was used directly for the next step. MS m/z: 329 (MH⁺).

### Step 3

### 4-Bromo-6-methoxyquinoline-3-carboxylic Acid

To a solution of ethyl 4-bromo-7-fluoro-6-methoxyquinoline-3-carboxylat (0.5 g wet in 30 mL of tetrahydrofuran) was added a solution of sodium hydroxide (0.1 g in 10 mL of water) slowly. The mixture was stirred overnight at room temperature. Condensed and acidified to pH 5 with concentrated hydrochloric acid. The white precipitate was collected by filtrate, washed with water and dried under vacuum to afford the pure title compound (317 mg). MS m/z: 283 (MH⁺).

### Step 4

### tert-Butyl 4-Bromo-7-fluoro-6-methoxyquinolin-3-ylcarbamate

A mixture of 4-bromo-6-methoxyquinoline-3-carboxylic acid (317 mg) and 4-methylmorpholine (118.3 mg) in 1,2-dichloroethane (10 mL) was stirred at room temperature for 15 minutes. Diphenyl phosphoryl azide (322 mg) was added dropwise to the clear solution and stirred for 30 minutes then refluxed for another 75 minutes. To the reaction mixture was added tert-butanol (10 mL) and refluxed overnight before cooled down. The reaction mixture was diluted with dichloromethane (300 mL), washed with water and brine, condensed. The residue was purified by column chromatography (20% ethyl acetate in petroleum ether) to give the title compound (192.4 mg). MS m/z: 372 (MH⁺).

### Step 5

### 4-Bromo-7-fluoro-6-methoxyquinolin-3-amine

To a solution of tert-butyl 4-bromo-7-fluoro-6-methoxyquinolin-3-ylcarbamate (192.4 mg) in dichloromethane (2 mL) was added trifluoroacetic acid (2 mL) and the mixture was stirred overnight at room temperature. Concentrated, residue was dissolved in ethyl acetate (200 mL) and washed subsequently with saturated sodium carbonate, water and brine. The ethyl acetate layer was dried over anhydrous sodium sulfate and condensed to give pure the title compound (127 mg). MS m/z: 272 (MH⁺).

### Step 6

### 4-Bromo-3,7-difluoro-6-methoxyquinoline

To an ice-cooled solution of 4-bromo-7-fluoro-6-methoxyquinolin-3-amine (127 mg) in dry tetrahydrofuran (10 mL) was added nitrosyl tetrafluoroborate (61 mg). The mixture was stirred at 0° C. for 50 minutes then filtrated. The solid cake was washed with cold tetrahydrofuran (1 mL) and dried by vacuum at room temperature to afford a brown powder. This powder was suspended in decaline was heated to 100° C. for 1 hour. Cooled down, diluted with petroleum ether (100 mL) and filtrated through a silica gel pad washed with petroleum ether to remove the decaline then washed with dichloromethane to afford a white solid (110 mg). MS m/z: 275 (MH⁺).

### Step 7

### tert-Butyl 1-(2-(3,7-Difluoro-6-methoxyquinolin-4-yl)ethyl)-2-oxabicyclo[2.2.2]octan-4-ylcarbamate

To a solution of B (100 mg) in anhydrous tetrahydrofuran (1.8 mL) was added a solution of 9-borabicyclo[3.3.1]nonane dimer (1.6 mL, 0.5 M in tetrahydrofuran) under cooling with ice, the mixture was stirred at room temperature for 1 hour. After quenching the reaction by adding water (1 drop) under cooling, the mixture was added 4-bromo-3,7-difluoro-6-methoxyquinoline (109.2 mg), tetrakis(triphenylphosphine)palladium (100 mg), tripotassium phosphate (0.6 g) and ethanol/water (2 mL, 4:1), and degassed. The mixture was heated at 70° C. for 12 hours and concentrated in vacuo. After dilution of the residue with ethyl acetate, the mixture was washed with water and brine, dried over anhydrous sodium sulfate, filtered, and then concentrated in vacuo gave the crude title compound, which was used directly.

### Step 8

### 1-(2-(3,7-Difluoro-6-methoxyquinolin-4-yl)ethyl)-2-oxabicyclo[2.2.2]octan-4-amine

To a solution of tert-butyl 1-(2-(3,7-difluoro-6-methoxyquinolin-4-yl)ethyl)-2-oxabicyclo[2.2.2]octan-4-ylcarbamate (120 mg, crude) in dichloromethane (2 mL) was added trifluoroacetic acid (2 mL) and the mixture was stirred at room temperature for 30 minutes and concentrated in vacuo. After dilution of the residue with water, the mixture was washed with methyl tert-butyl ether twice. The aqueous layer was adjusted to pH 13 by addition of aqueous sodium carbonate solution and extract twice with ethyl acetate. The combined ethyl acetate layer was washed with brine, dried over anhydrous sodium sulfate and condensed to give the title compound. MS m/z: 349 (MH⁺).

### Step 9

### 6-((1-(2-(3,7-Difluoro-6-methoxyquinolin-4-yl)ethyl)-2-oxabicyclo[2.2.2]octan-4-ylamino)methyl)-2H-pyrido[3,2-b][1,4]oxazin-3(4H)-one

A mixture of 1-(2-(3,7-difluoro-6-methoxyquinolin-4-yl)ethyl)-2-oxabicyclo[2.2.2]octan-4-amine (99 mg) and I (76 mg) in anhydrous N,N-dimethylformamide (3.5 mL) was added acetic acid (0.5 mL) was stirred at room temperature for 30 minutes. The resulting solution was added three times of sodium triacetoxyborohydride (118.8 mg) and stirred at room temperature for overnight. The mixture was concentrated in vacuo. After dilution of the residue with dichloromethane, the mixture was washed with saturated sodium carbonate solution, water and brine. The organic extracts were dried over anhydrous sodium sulfate then concentrated in vacuo. The residue was purified by prep-TLC (dichloromethane:methanol=10:1) to gave the title compound (72 mg). To a solution of the title compound (72 mg) in dichloromethane (2 mL) and ethanol (0.5 mL) was added a solution of hydrogen chloride (26 uL, 4 M in dioxane) under cooling with ice, the mixture was stirred at room temperature for 2 hours and concentrated in vacuo. Treatment of the residue with ethanol gave the title compound as its HCl salt.

¹H NMR (MeOD): δ 1.83-1.87 (m, 2H), 1.96-1.98 (m, 2H), 2.16-2.20 (m, 6H), 3.39-3.40 (m, 2H), 4.09 (s, 2H), 4.15 (s, 3H), 4.24 (s, 2H), 4.69 (s, 2H), 7.12 (d, J=8.0 Hz, 1H), 7.36 (d, J=8.0 Hz, 1H), 7.83 (d, J=7.6 Hz, 1H), 7.91 (d, J=10.4 Hz, 1H), 9.15 (s, 1H).

MS m/z: 511 (MH⁺).

### Example 142

### 2-((1-(2-(3-Fluoro-6-methoxy-1,5-naphthyridin-4-yl)ethyl)-2-oxabicyclo[2.2.2]octan-4-ylamino)methyl)-6H-pyrimido[5,4-b][1,4]oxazin-7(8H)-one

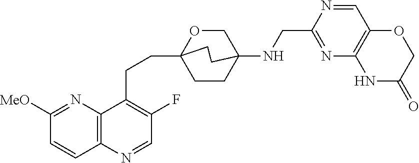 [SMILES: COc1ccc2ncc(F)c(CCC34CCC(NCc5ncc6c(n5)NC(=O)CO6)(CC3)CO4)c2n1]

### Step 1

### Ethyl 2-(Tetrahydro-2H-pyran-2-yloxy)acetate

To a stirred solution of ethyl hydroxyacetate (35.3 g) containing a few crystals of p-toluene sulfonic acid, dihydropyran (30.0 g) was added dropwise. After stirring overnight at room temperature, the mixture was diluted with dichloromethane (200 mL) and washed with a sodium hydrogencarbonate solution. The organic layer was separated and dried followed by evaporation of the dichloromethane. The residue was distilled under high vacuum to give the title compound (32 g) as a clear liquid.

¹H NMR (CDCl₃): δ 1.20-1.32 (m, 3H), 1.50-1.58 (m, 3H), 1.70-1.92 (m, 3H), 3.45-3.55 (m, 1H), 3.80-3.90 (m, 1H), 4.16-4.24 (m, 4H), 4.70-4.79 (m, 1H).

### Step 2

### Cinnamimidamide

A solution of (2E)-3-phenylprop-2-enenitrile (25 g) in anhydrous ethanol (200 mL) was cooled to 0° C. and hydrogen chloride gas bubbled through the solution for 30 minutes. The solution was stirred at ambient temperature for 16 hours and then concentrated under vacuum. The residue was dissolved in ethanol (100 mL), cooled to 0° C. and a solution of ammonia/methanol (7 M, 69 mL) was added dropwise through an addition funnel. Once added, the solution was allowed to warm to ambient temperature and the resulting ammonium chloride was filtered off. The solution was concentrated under vacuum and the residue was dissolved in water, washed with ethyl acetate. The aqueous layer was dried to give the title compound (20 g), which was used next step without further purification. MS m/z: 147 (MH⁺).

### Step 3

### (E)-2-Styryl-5-(tetrahydro-2H-pyran-2-yloxy)pyrimidin-4(3H)-one

A solution of the product from Step 1 (10 g) in tetrahydrofuran (200 mL) and dry ethyl formate (39 g) was added sodium hydride (3.8 g) slowly. The reaction mixture was concentrated to dryness to give a pale yellow solid. The solid was added to a methanol/ethanol (200 mL/200 mL) solution of the product from Step 2 (7.8 g), the subsequent mixture was heated at 80° C. for 4 hours. The resulting material was poured into dichloromethane (100 mL) containing silica gel (30 g) and evaporated. The residue was purified by column chromatography silica gel to give the title compound (5 g) as a pale yellow solid. MS m/z: 299 (MH⁺).

### Step 4

### (E)-2-Styryl-5-(tetrahydro-2H-pyran-2-yloxy)pyrimidin-4-yl trifluoromethanesulfonate

To a suspension of (E)-2-styryl-5-(tetrahydro-2H-pyran-2-yloxy)pyrimidin-4(3H)-one (2.04 g) in dichloromethane (25 mL) was added pyridine (1.22 mL). After cooling to −78° C., trifluoromethanesulphonic anhydride (1.38 mL) was slowly added via dropwise addition. The reaction was maintained at −78° C. for 10 minutes, after which time the cooling bath was replaced with an ice-water bath and the reaction was stirred for an additional 30 minutes. The reaction mixture was poured into water and the aqueous phase was extracted with dichloromethane. The organic phase was then washed with water, saturated sodium hydrogencarbonate solution and brine. The organic phase was dried over sodium sulfate, filtered, and concentrated under vacuum to provide a dark reddish oil which was used directly in the next step. MS m/z: 431 (MH⁺).

### Step 5

### (E)-2-Styryl-5-(tetrahydro-2H-pyran-2-yloxy)pyrimidin-4-amine

Crude (E)-2-styryl-5-(tetrahydro-2H-pyran-2-yloxy)pyrimidin-4-yltrifluoromethanesulfonate (2.9 g) was reacted with a 0.5 M solution of ammonia in 1,4-dioxane (136 mL) in a pressure bottle at 60° C. for 24 hours. The reaction was concentrated under vacuum, the residue was taken up in dichloromethane and washed with water, saturated aq. sodium hydrogencarbonate and brine. The organic phase was dried over sodium sulfate, filtered and concentrated. The crude residue was purified by column chromatography (silica gel) using a methanol/dichloromethane gradient to yield the desired compound as a tan solid (1.28 g). MS m/z: 298 (MH⁺).

### Step 6

### (E)-4-Amino-2-styrylpyrimidin-5-ol

A suspension of (E)-2-styryl-5-(tetrahydro-2H-pyran-2-yloxy)pyrimidin-4-amine (1.28 g) in methanol (25 mL) was heated in a 50° C. oil bath until fully dissolved. To this was added a solution of hydrogen chloride (0.1 mL, 4 M in 1,4-dioxane) and the reaction was heated at 50° C. for 1.5 hour. At this time, LCMS indicated little progression, therefore an additional solution of hydrogen chloride (1.1 mL, 4 M in 1,4-dioxane) was added and heating was continued for 3 hours. The reaction was allowed to cool to room temperature resulting in the formation of a white precipitate. The solvent was removed under vacuum and the resulting tan solid was dried under high vacuum over night yielding the title compound (1.08 g). This material was used without further purification. MS m/z: 214 (MH⁺).

### Step 7

### 2-[(E)-2-Phenylethenyl]-6H-pyrimido[5,4-b][1,4]oxazin-7(8H)-one

To a suspension of (E)-4-amino-2-styrylpyrimidin-5-ol (214 mg) in absolute ethanol (5 mL) was added potassium tert-butoxide (224 mg) at room temperature. After stirring for 5 minutes, ethyl bromoacetate (0.1 mL) was added dropwise and the reaction was stirred for 18 hours. The solvent was evaporated and the residue was taken up in 10% methanol-chloroform and a small amount of water. The layers were separated and the aqueous phase was extracted with 10% methanol-chloroform. The combined organic extracts were concentrated and the resulting solid was triturated with ethyl acetate. The white solid was collected by filtrate (106 mg). MS m/z: 254 (MH⁺).

### Step 8

### 7-Oxo-7,8-dihydro-6H-pyrimido[5,4-b][1,4]oxazine-2-carbaldehyde

To a suspension of 2-[(E)-2-phenylethenyl]-6H-pyrimido[5,4-b][1,4]oxazin-7(8H)-one (106 mg) in 1,4-dioxane (12 mL) and water (3 mL) was added sodium periodate (357 mg) and osmium tetroxide (0.1 mL, 4% wt in water) and the reaction mixture was stirred at room temperature. After 2 hours, an additional 1,4-dioxane (3 mL) and sodium periodate (180 mg) were added. After a total of 7.5 hours, the reaction was capped and stored in a freezer for the weekend. After warming to room temperature, additional osmium tetroxide (0.1 mL, 4% wt in water) was added and the reaction was stirred for an additional 4 hours. The solvent was evaporated to give a white solid which was dissolved in dichloromethane and water. The aqueous layer was extracted with 10% methanol-dichloromethane (6 times). The combined organic extracts were dried over sodium sulfate, filtered, and concentrated to give a light tan solid (92 mg).

¹H NMR (DMSO-d₆): δ 4.81 (s, 2H), 8.47 (s, 1H), 9.78 (s, 1H).

MS m/z: 180 (MH⁺).

### Step 9

### 2-((1-(2-(3-Fluoro-6-methoxy-1,5-naphthyridin-4-yl)ethyl)-2-oxabicyclo[2.2.2]octan-4-ylamino)methyl)-6H-pyrimido[5,4-b][1,4]oxazin-7(8H)-one

A solution of 7-oxo-7,8-dihydro-6H-pyrimido[5,4-b][1,4]oxazine-2-carbaldehyde (27 mg) and 1-[2-(3-fluoro-6-methoxy-1,5-naphthyridin-4-yl)ethyl]-2-oxabicyclo[2.2.2]octan-4-amine (0.15 mmol) in N,N-dimethylformamide:acetic acid=7:1 (5 mL) was stirred at room temperature for 30 minutes and then sodium triacetoxyborohydride (64 mg) was added. The mixture was stirred at room temperature for another 5 hours and then purified by prep-HPLC to give the desired product.

¹H NMR (CDCl₃): δ 1.80-2.20 (m, 10H), 3.20-3.28 (m, 2H), 4.00 (s, 2H), 4.10 (s, 3H), 4.30 (s, 2H), 4.78 (s, 2H), 7.15-7.20 (m, 1H), 8.16-8.22 (m, 1H), 8.28 (s, 1H), 8.62 (s, 1H).

MS m/z: 495 (MH⁺).

### Example 143

### 2-((1-(2-(3-Fluoro-6-methoxy-1,5-naphthyridin-4-yl)-1-hydroxyethyl)-2-oxabicyclo[2.2.2]octan-4-ylamino)methyl)-6H-pyrimido[5,4-b][1,4]oxazin-7(8H)-one

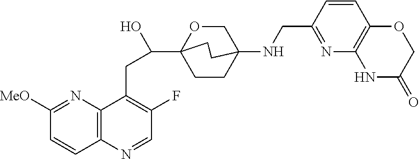 [SMILES: COc1ccc2ncc(F)c(CC(O)C34CCC(NCc5ccc6c(n5)NC(=O)CO6)(CC3)CO4)c2n1]

A solution of 7-oxo-7,8-dihydro-6H-pyrimido[5,4-b][1,4]oxazine-2-carbaldehyde (27 mg) and tert-butyl 1-(2-(3-fluoro-6-methoxy-1,5-naphthyridin-4-yl)-1-hydroxyethyl)-2-oxabicyclo[2.2.2]octan-4-ylcarbamate (0.15 mmol) in N,N-dimethylformamide:acetic acid=7:1 (5 mL) was stirred at room temperature for 30 minutes and then sodium triacetoxyborohydride (64 mg) was added. The mixture was stirred at room temperature for another 5 hours and then purified by prep-HPLC to give the desired products.

¹H NMR (CDCl₃): δ 1.90-2.40 (m, 8H), 3.20-3.28 (m, 1H), 3.40-3.58 (m, 1H), 3.95-4.05 (m, 3H), 4.10 (s, 3H), 4.30 (s, 2H), 4.78 (s, 2H), 7.15-7.20 (m, 1H), 8.16-8.28 (m, 2H), 8.62 (s, 1H).

MS m/z: 511 (MH⁺).

### Example 144

### N-((6,7-Dihydro-[1,4]dioxino[2,3-d]pyrimidin-2-yl)methyl)-1-(2-(3-fluoro-6-methoxy-1,5-naphthyridin-4-yl)ethyl)-2-oxabicyclo[2.2.2]octan-4-amine

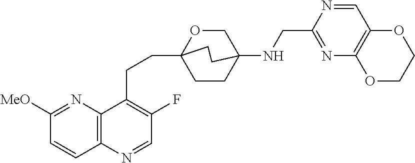 [SMILES: COc1ccc2ncc(F)c(CCC34CCC(NCc5ncc6c(n5)OCCO6)(CC3)CO4)c2n1]

### Step 1

### (E)-5-Hydroxy-2-styrylpyrimidin-4(3H)-one

A solution of 2-[(E)-2-phenylethenyl]-5-(tetrahydro-2H-pyran-2-yloxy)pyrimidin-4(3H)-one (2.98 g) in hydrogen chloride solution (50 mL, 4 M in 1,4-dioxane) was stirred at 50° C. for 90 minutes. The mixture was diluted with water and the pH was adjusted to 5 with saturated sodium hydrogencarbonate. Extracted with ethyl acetate twice, the organic extracts were washed with brine, dried over anhydrous sodium sulfate and condensed to afford a white solid (1.83 g). MS m/z: 215 (MH⁺).

### Step 2

### (E)-2-Styryl-6,7-dihydro-[1,4]dioxino[2,3-d]pyrimidine

A solution of (E)-5-hydroxy-2-styrylpyrimidin-4(3H)-one (642 mg) in N,N-dimethylformamide (100 mL) was added sodium hydride (1.2 g, 60% in mineral oil) at 0° C. and stirred for 60 minutes. Then 1,2-dibromoethane was added slowly and the mixture was stirred overnight at room temperature. The reaction mixture was quenched with water and diluted with ethyl acetate (200 mL), washed with water and brine, condensed. The residue was purified by column chromatography (25% ethyl acetate in petroleum ether) to afford a white powder (0.27 g). MS m/z: 241 (MH⁺).

### Step 3

### 6,7-Dihydro-[1,4]dioxino[2,3-d]pyrimidine-2-carbaldehyde

To a solution of (E)-2-styryl-6,7-dihydro-[1,4]dioxino[2,3-d]pyrimidine (300 mg) in dichloromethane/methanol (40 mL, v/v=1:1) was bubbled ozone at −78° C. for 15 minutes to get a blue solution. Nitrogen was bubbled for another 15 minutes at −78° C. to remove excess of ozone before dimethyl sulfide (1 mL) was added. The mixture and warmed to room temperature and stirred for 30 minutes and condensed. The residue was purified by prep-TLC (petroleum ether:ethyl acetate=1:1) to afford a white powder (119 mg).

¹H NMR (CDCl₃): δ 4.33-4.36 (m, 2H), 4.53-4.57 (m, 2H), 8.36 (s, 1H), 9.85 (s, 1H).

MS m/z: 167 (MH⁺).

### Step 4

### N-((6,7-Dihydro-[1,4]dioxino[2,3-d]pyrimidin-2-yl)methyl)-1-(2-(3-fluoro-6-methoxy-1,5-naphthyridin-4-yl)ethyl)-2-oxabicyclo[2.2.2]octan-4-amine

A mixture of 1-[2-(3-fluoro-6-methoxy-1,5-naphthyridin-4-yl)ethyl]-2-oxabicyclo[2.2.2]octan-4-amine (33.1 mg) and 6,7-dihydro-[1,4]dioxino[2,3-d]pyrimidine-2-carbaldehyde (24.9 mg) in anhydrous N,N-dimethylformamide (3.5 mL) was added acetic acid (0.5 mL) was stirred at room temperature for 30 minutes. The resulting solution was added three times of sodium triacetoxyborohydride (31.8 mg) and stirred at room temperature overnight. The mixture was concentrated in vacuo. After dilution of the residue with dichloromethane, the mixture was washed with saturated sodium carbonate solution, water and brine. The organic extracts were dried over anhydrous sodium sulfate then concentrated in vacuo. The residue was purified by prep-TLC (dichloromethane:methanol=10:1) to afford a solid. To a solution of this solid (30 mg) in dichloromethane (2 mL) and ethanol (0.5 mL) was added a solution of hydrogen chloride (15 uL, 4 M in 1,4-dioxane) under cooling with ice, the mixture was stirred at room temperature for 2 hours and concentrated in vacuo. Treatment of the residue with ethanol gave the title compound.

¹H NMR (MeOD): δ 1.80-1.87 (m, 2H), 1.91-1.99 (m, 2H), 2.01-2.19 (m, 6H), 3.33-3.38 (m, 2H), 4.00 (s, 2H), 4.14 (s, 3H), 4.27 (s, 2H), 4.34-4.36 (m, 2H), 4.56-4.58 (m, 2H), 7.39 (d, J=9.2 Hz, 1H), 8.26 (s, 1H), 8.33 (d, J=9.2 Hz, 1H), 8.96 (s, 1H).

MS m/z: 482 (MH⁺).

### Example 145

### (R)-1-(4-((6,7-Dihydro-[1,4]dioxino[2,3-d]pyrimidin-2-yl)methylamino)-2-oxabicyclo[2.2.2]octan-1-yl)-2-(3-fluoro-6-methoxy-1,5-naphthyridin-4-yl)ethanol

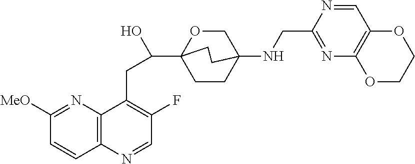 [SMILES: COc1ccc2ncc(F)c(CC(O)C34CCC(NCc5ncc6c(n5)OCCO6)(CC3)CO4)c2n1]

### Step 1

### (R)-1-(4-Amino-2-oxabicyclo[2.2.2]octan-1-yl)-2-(3-fluoro-6-methoxy-1,5-naphthyridin-4-yl)ethanol

To a solution of tert-butyl 1-(2-(3-fluoro-6-methoxy-1,5-naphthyridin-4-yl)-1-hydroxyethyl)-2-oxabicyclo[2.2.2]octan-4-ylcarbamate (89 mg) in dichloromethane (2 mL) was added trifluoroacetic acid (2 mL) and the mixture was stirred at room temperature for 30 minutes and concentrated in vacuo. After dilution of the residue with water, the mixture was washed with methyl tert-butyl ether twice. The aqueous layer was adjusted to pH 13 by addition of aqueous sodium carbonate solution and extract twice with ethyl acetate. The combined ethyl acetate layer was washed with brine, dried over anhydrous sodium sulfate and condensed to give the title compound (65 mg). MS m/z: 348 (MH⁺).

### Step 2

### (R)-1-(4-((6,7-Dihydro-[1,4]dioxino[2,3-d]pyrimidin-2-yl)methylamino)-2-oxabicyclo[2.2.2]octan-1-yl)-2-(3-fluoro-6-methoxy-1,5-naphthyridin-4-yl)ethanol

A mixture of (R)-1-(4-amino-2-oxabicyclo[2.2.2]octan-1-yl)-2-(3-fluoro-6-methoxy-1,5-naphthyridin-4-yl)ethanol (34.7 mg) and 6,7-dihydro[1,4]dioxino[2,3-d]pyrimidine-2-carbaldehyde (24.9 mg) in anhydrous N,N-dimethylformamide (3.5 mL) was added acetic acid (0.5 mL) was stirred at room temperature for 30 minutes. The resulting solution was added three times of sodium triacetoxyborohydride (31.8 mg) and stirred at room temperature overnight. The mixture was concentrated in vacuo. After dilution of the residue with dichloromethane, the mixture was washed with saturated sodium carbonate solution, water and brine. The organic extracts were dried over anhydrous sodium sulfate then concentrated in vacuo. The residue was purified by prep-TLC (dichloromethane:methanol=10:1) to afford a solid. To a solution of this solid (16 mg) in dichloromethane (2 mL) and ethanol (0.5 mL) was added a solution of hydrogen chloride (8 μL, 4 M in 1,4-dioxane) under cooling with ice, the mixture was stirred at room temperature for 2 hours and concentrated in vacuo. Treatment of the residue with ethanol gave the title compound.

¹H NMR (MeOD): δ 1.95-2.06 (m, 1H), 2.09-2.21 (m, 6H), 2.25-2.31 (m, 1H), 3.33-3.39 (m, 1H), 3.65 (d, J=12.0 Hz, 1H), 3.97-4.03 (m, 3H), 4.15 (s, 3H), 4.26 (s, 2H), 4.42-4.46 (m, 2H), 4.53-4.61 (m, 2H), 7.40 (d, J=9.2 Hz, 1H), 8.26 (s, 1H), 8.36 (d, J=9.2 Hz, 1H), 9.00 (s, 1H).

MS m/z: 498 (MH⁺).

### Example 146

### N-((2,3-Dihydro-[1,4]dioxino[2,3-b]pyridin-6-yl)methyl)-1-(2-(3-fluoro-6-methoxy-1,5-naphthyridin-4-yl)ethyl)-2-oxabicyclo[2.2.2]octan-4-amine

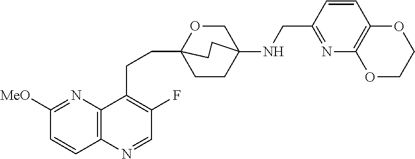 [SMILES: COc1ccc2ncc(F)c(CCC34CCC(NCc5ccc6c(n5)OCCO6)(CC3)CO4)c2n1]

### Step 1

### 6-Bromo-2-chloropyridin-3-ol

A mixture of 2-chloropyridin-3-ol (12.9 g) and sodium acetate (8.2 g) in acetic acid (150 mL) was added bromine (16 g) slowly. The mixture was stirred overnight at room temperature then poured into ice-water. Extracted with ethyl acetate twice and the organic extract was washed with brine. Condensed, the residue was purified by column chromatography (eluted with 25% ethyl acetate in petroleum ether) to afford the title compound as a solid (8.4 g).

¹H NMR (CDCl₃): δ 7.15 (d, J=8.0 Hz, 1H), 7.28 (d, J=8.0 Hz, 1H), 7.53 (s, 1H).

### Step 2

### 2-(6-Bromo-2-chloropyridin-3-yloxy)ethanol

6-Bromo-2-chloropyridin-3-ol (8.2 g) was added to 1 N sodium hydroxide solution (100 mL) at room temperature and stirred for 30 minutes. 2-Bromoethanol (10.1 g) was added and the mixture was refluxed for 4 hours. The mixture was extracted with ethyl acetate twice and the organic extracts were washed with brine and condensed. The residue was purified by column chromatography (eluted with 50% ethyl acetate in petroleum ether) to afford a solid (9.4 g).

¹H NMR (CDCl₃): δ 2.18 (t, J=6.4 Hz, 1H), 3.99-4.03 (m, 2H), 4.12-4.14 (m, 2H), 7.13 (d, J=8.0 Hz, 1H), 7.35 (d, J=8.0 Hz, 1H).

MS m/z: 254 (MH⁺).

### Step 3

### 6-Bromo-2,3-dihydro-[1,4]dioxino[2,3-b]pyridine

A mixture of 2-(6-bromo-2-chloropyridin-3-yloxy)ethanol (7.9 g), potassium hydroxide (2.6 g, 85%) and 18-crown-6 (1.0 g) in toluene (150 mL) was refluxed for 45 minutes. The mixture was diluted with ethyl acetate and washed with water and brine. Condensed, the residue was purified by column chromatography (eluted with 70% ethyl acetate in petroleum ether) to afford a white solid (3.1 g).

¹H NMR (CDCl₃): δ 4.22-4.24 (m, 2H), 4.40-4.42 (m, 2H), 6.99 (d, J=8.0 Hz, 1H), 7.04 (d, J=8.0 Hz, 1H).

MS m/z: 216 (MH⁺).

### Step 4

### 6-Vinyl-2,3-dihydro-[1,4]dioxino[2,3-b]pyridine

A mixture of 6-bromo-2,3-dihydro-[1,4]dioxino[2,3-b]pyridine (1.1 g), potassium vinyltrifluoroborate (0.8 g) and PdCl₂(dppf) (100 mg) in ethanol (20 mL) and triethanolamine (20 mL) was refluxed under nitrogen for 4 hours. Condensed, the residue was purified by column chromatography (petroleum ether:ethyl acetate=1:1) to afford the title compound (0.7 g).

¹H NMR (CDCl₃): δ 4.18-4.20 (m, 2H), 4.36-4.38 (m, 2H), 5.23-5.27 (m, 1H), 5.98-6.03 (m, 1H), 6.54-6.61 (m, 1H), 6.82 (d, J=8.0 Hz, 1H), 7.06 (d, J=8.0 Hz, 1H).

MS m/z: 164 (MH⁺).

### Step 5

### 2,3-Dihydro-[1,4]dioxino[2,3-b]pyridine-6-carbaldehyde

To a solution of 6-vinyl-2,3-dihydro-[1,4]dioxino[2,3-b]pyridine (700 mg) in dichloromethane/methanol (20 mL, v/v=1:1) was bubbled ozone at −78° C. for 15 minutes to get a blue solution. Nitrogen was bubbled for another 15 minutes at −78° C. to remove excess of ozone before dimethyl sulfide (1 mL) was added. The mixture and warmed to room temperature and stirred for 30 minutes and condensed. The residue was purified by prep-TLC (petroleum ether:ethyl acetate=1:1) to afford a white powder (520 mg). MS m/z: 166 (MH⁺).

### Step 6

### N-((2,3-Dihydro-[1,4]dioxino[2,3-b]pyridin-6-yl)methyl)-1-(2-(3-fluoro-6-methoxy-1,5-naphthyridin-4-yl)ethyl)-2-oxabicyclo[2.2.2]octan-4-amine

A mixture of 1-[2-(3-fluoro-6-methoxy-[1,5]naphthyridin-4-yl)-ethyl]-2-oxa-bicyclo[2.2.2]oct-4-ylamine (71 mg) and 2,3-dihydro-[1,4]dioxino[2,3-b]pyridine-6-carbaldehyde (67 mg) in anhydrous N,N-dimethylformamide (3.5 mL) was added acetic acid (0.5 mL) was stirred at room temperature for 30 minutes. The resulting solution was added three times of sodium triacetoxyborohydride (106 mg) and stirred at room temperature overnight. The mixture was concentrated in vacuo. After dilution of the residue with dichloromethane, the mixture was washed with saturated sodium carbonate solution, water and brine. The organic extracts were dried over anhydrous sodium sulfate then concentrated in vacuo. The residue was purified by prep-TLC (dichloromethane:methanol=10:1) to afford a solid. To a solution of this solid (66 mg) in dichloromethane (2 mL) and ethanol (0.5 mL) was added a solution of hydrogen chloride (34 uL, 4 M in 1,4-dioxane) under cooling with ice, the mixture was stirred at room temperature for 2 hours and concentrated in vacuo. Treatment of the residue with ethanol gave the title compound.

¹H NMR (MeOD): δ 1.86-1.91 (m, 4H), 2.07-2.18 (m, 6H), 3.46-3.49 (m, 2H), 3.95 (s, 2H), 4.15 (s, 2H), 4.18 (s, 3H), 4.27-4.29 (m, 2H), 4.43-4.46 (m, 2H), 7.07 (d, J=8.0 Hz, 1H), 7.32 (d, J=8.0 Hz, 1H), 7.53 (d, J=9.2 Hz, 1H), 8.42 (d, J=9.2 Hz, 1H), 9.17 (s, 1H).

MS m/z: 481 (MH⁺).

### Example 147

### 1-(4-((2,3-Dihydro-[1,4]dioxino[2,3-b]pyridin-6-yl)methylamino)-2-oxabicyclo[2.2.2]octan-1-yl)-2-(3-fluoro-6-methoxy-1,5-naphthyridin-4-yl)ethanol

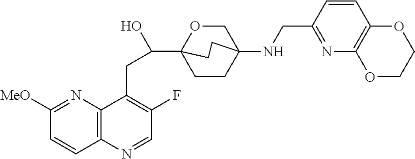 [SMILES: COc1ccc2ncc(F)c(CC(O)C34CCC(NCc5ccc6c(n5)OCCO6)(CC3)CO4)c2n1]

### Step 1

### 1-(4-((2,3-Dihydro-[1,4]dioxino[2,3-b]pyridin-6-yl)methylamino)-2-oxabicyclo[2.2.2]octan-1-yl)-2-(3-fluoro-6-methoxy-1,5-naphthyridin-4-yl)ethanol

A mixture of 1-(4-amino-2-oxabicyclo[2.2.2]oct-1-yl)-2-(3-fluoro-6-methoxy-1,5-naphthyridin-4-yl)ethanol (60 mg) and 2,3-dihydro[1,4]dioxino[2,3-b]pyridine-6-carbaldehyde (42 mg) in anhydrous N,N-dimethylformamide (3.5 mL) was added acetic acid (0.5 mL) was stirred at room temperature for 30 minutes. The resulting solution was added three times of sodium triacetoxyborohydride (72 mg) and stirred at room temperature for overnight. The mixture was concentrated in vacuo. After dilution of the residue with dichloromethane, the mixture was washed with saturated sodium carbonate solution, water and brine. The organic extracts were dried over anhydrous sodium sulfate then concentrated in vacuo. The residue was purified by prep-TLC (dichloromethane:methanol=10:1) to give the title compound (36 mg). To a solution of the title compound (36 mg) in dichloromethane (2 mL) and ethanol (0.5 mL) was added a solution of hydrogen chloride (18 μL, 4 M in 1,4-dioxane) under cooling with ice, the mixture was stirred at room temperature for 2 hours and concentrated in vacuo. Treatment of the residue with ethanol gave the title compound as its HCl salt.

¹H NMR (MeOD): δ 2.01-2.11 (m, 7H), 2.29-2.30 (m, 1H), 2.88 (s, 4H), 3.41-3.46 (m, 1H), 4.01 (s, 2H), 4.19 (s, 3H), 4.29 (s, 2H), 4.46 (s, 2H), 7.12 (d, J=8.0 Hz, 1H), 7.33 (d, J=8.0 Hz, 1H), 7.53 (d, J=8.8 Hz, 1H), 8.45 (d, J=8.8 Hz, 1H), 9.19 (s, 1H).

MS m/z: 497 (MH⁺).

### Example 148

### 7-((1-(2-(3-Fluoro-6-methoxy-1,5-naphthyridin-4-yl)ethyl)-2-oxabicyclo[2.2.2]octan-4-ylamino)methyl)-1H-pyrido[3,4-b][1,4]oxazin-2(3H)-one

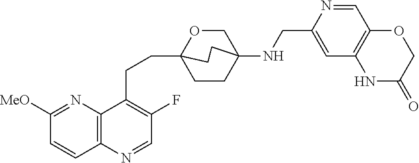 [SMILES: COc1ccc2ncc(F)c(CCC34CCC(NCc5cc6c(cn5)OCC(=O)N6)(CC3)CO4)c2n1]

### Step 1

### Methyl 2-(6-Bromopyridin-3-yloxy)acetate

A solution of 6-bromopyridin-3-ol (1.74 g) and potassium carbonate (2.76 g) in acetone (30 mL) was added dropwise chloroacetic methyl ester (1.08 g), and the resulting mixture was stirred under reflux for 15 hours. Then the mixture was filtered and the filtrate was concentrated in vacuo to afford the crude product. The crude product was purified via column chromatography affording the title compound (1.23 g). MS m/z: 246 (MH⁺).

### Step 2

### 2-Bromo-5-(2-methoxy-2-oxoethoxy)pyridine 1-Oxide

To a mixture of methyl 2-(6-bromopyridin-3-yloxy)acetate (1.2 g) in dichloromethane (20 mL) was added m-chloroperbenzoic acid (1.72 g) and the resulting mixture was stirred for 18 hours. The mixture was extracted by dichloromethane twice, and the organic layers were washed with saturated sodium sulfite solution twice. Concentrated in vacuo, the crude title compound (0.65 g) was obtained, which was used for next step directly. MS m/z: 262 (MH⁺)

### Step 3

### 2-Bromo-5-(2-methoxy-2-oxoethoxy)-4-nitropyridine 1-Oxide

The N-oxide 2-bromo-5-(2-methoxy-2-oxoethoxy)pyridine 1-oxide (2.69 g) was dissolved in sulfuric acid (4 mL) at 0° C., and then nitric acid (2 mL) was added slowly over several minutes. The reaction mixture was then placed in an oil bath heated to 40° C., then the temperature was slowly raised to 75° C. over 1 hour and then maintained there for 2 hours. The mixture was slowly poured over ice and adjusted to pH 9. Water removed in vacuo and the residue was dissolved in methanol (50 mL) and treated with sulfuric acid (1 mL), the mixture was heated at 70° C. for 2 hours, and concentrated. The residue was treated with 1 N sodium hydroxide solution (40 mL) and ethyl acetate. The organic layer was dried over sodium sulfate and concentrated in vacuo to give the title compound (1 g). MS m/z: 306 (MH⁺).

### Step 4

### 7-Bromo-1H-pyrido[3,4-b][1,4]oxazin-2(3H)-one

To a stirred solution of 2-bromo-5-(2-methoxy-2-oxoethoxy)-4-nitropyridine 1-oxide (1.8 g) in ethanol (100 mL) was added iron powder (1.8 g) and acetic acid (3 mL), and the resulting mixture was stirred under reflux for 2 hours, and then filtered. The filtrate was concentrated in vacuo to afford the crude product. The crude product was partitioned between water and ethyl acetate, the organic layer was dried and concentrated in vacuo to afford the title compound (0.6 g). MS m/z: 229 (MH⁺).

### Step 5

### (E)-7-Styryl-1H-pyrido[3,4-b][1,4]oxazin-2(3H)-one

To a degassed solution of 7-bromo-1H-pyrido[3,4-b][1,4]oxazin-2(3H)-one (600 mg) in 1,4-dioxane (20 mL) and water (4 mL) was added phenylvinylboronic acid (300 mg), potassium carbonate (690 mg) and tetrakis(triphenylphosphine)palladium (60 mg), the mixture was heated at reflux for 24 hours. After dilution of the mixture with water (720 mL), the resulting precipitates were collected by filtrate gave the title compound (400 mg). MS m/z: 253 (MH⁺)

### Step 6

### 2-Oxo-2,3-dihydro-1H-pyrido[3,4-b][1,4]oxazine-7-carbaldehyde

A suspension of (E)-7-styryl-1H-pyrido[3,4-b][1,4]oxazin-2(3H)-one (400 mg) in dichloromethane (30 mL) and methanol (10 mL) was bubbled with ozone at 71° C. until a pale blue color appeared. The excess ozone was removed by bubbling air through the suspension for 30 minutes. Dimethyl sulfide (1 mL) was added to the suspension. The mixture was stirred at room temperature overnight and concentrated in vacuo to give the title compound (143.2 mg).

MS m/z: 179 (MH⁺).

### Step 7

### 7-((1-(2-(3-Fluoro-6-methoxy-1,5-naphthyridin-4-yl)ethyl)-2-oxabicyclo[2.2.2]octan-4-ylamino)methyl)-1H-pyrido[3,4-b][1,4]oxazin-2(3H)-one

A solution of 2-oxo-2,3-dihydro-1H-pyrido[3,4-b][1,4]oxazine-7-carbaldehyde (36 mg) and 1-[2-(3-fluoro-6-methoxy-1,5-naphthyridin-4-yl)ethyl]-2-oxabicyclo[2.2.2]octan-4-amine (66 mg) in N,N-dimethylformamide:acetic acid=7:1 (5 mL) was stirred at room temperature for 30 minutes and then sodium triacetoxyborohydride (71 mg) was added. The mixture was stirred at room temperature for another 5 hours, purified by prep-HPLC to give the title compound (30 mg).

¹H NMR (MeOD): δ 1.81-2.20 (m, 10H), 3.31 (s, 2H), 4.11 (s, 3H), 4.25 (s, 2H), 4.72 (s, 2H), 6.99 (s, 1H), 7.18 (d, J=9.3 Hz, 1H), 8.19-8.22 (m, 2H), 8.62 (s, 1H).

MS m/z: 494 (MH⁺).

### Example 149

### 7-((1-(2-(3-Fluoro-6-methoxy-1,5-naphthyridin-4-yl)-1-hydroxyethyl)-2-oxabicyclo[2.2.2]octan-4-ylamino)methyl)-1H-pyrido[3,4-b][1,4]oxazin-2(3H)-one

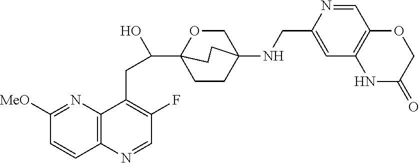 [SMILES: COc1ccc2ncc(F)c(CC(O)C34CCC(NCc5cc6c(cn5)OCC(=O)N6)(CC3)CO4)c2n1]

A solution of 2-oxo-2,3-dihydro-1H-pyrido[3,4-b][1,4]oxazine-7-carbaldehyde (36 mg) and 1-(4-amino-2-oxabicyclo[2.2.2]oct-1-yl)-2-(3-fluoro-6-methoxy-1,5-naphthyridin-4-yl)ethanol (68 mg) in N,N-dimethylformamide:acetic acid=7:1 (5 mL) was stirred at room temperature for 30 minutes and then sodium triacetoxyborohydride (71 mg) was added. The mixture was stirred at room temperature for overnight and then purified by prep-HPLC to give the title compound (31 mg).

¹H NMR (MeOD): δ 2.38-2.74 (m, 8H), 3.89 (s, 1H), 4.36-4.39 (m, 5H), 5.24 (s, 2H), 7.53 (s, 1H), 7.71 (d, J=9.1 Hz, 1H), 8.65 (s, 1H), 8.74 (d, J=9.1 Hz, 1H), 9.16 (s, 1H).

MS m/z: 510 (MH⁺).

### Example 150

### 6-((1-(2-(6,8-Dimethoxy-1,5-naphthyridin-4-yl)ethyl)-2-oxabicyclo[2.2.2]octan-4-ylamino)methyl)-2H-pyrido[3,2-b][1,4]oxazin-3(4H)-one

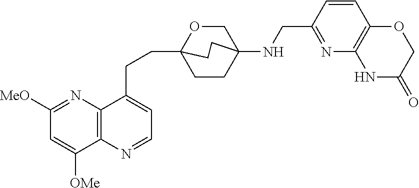 [SMILES: COc1cc(OC)c2nccc(CCC34CCC(NCc5ccc6c(n5)NC(=O)CO6)(CC3)CO4)c2n1]

### Step 1

### 2,4-Dichloro-5-nitropyridine

4-Chloro-5-nitropyridin-2-ol (8.4 g) was added to phosphorous oxychloride (20 mL) at ambient temperature then the reaction was heated to reflux for 1 hour before it was cooled to ambient temperature. The mixture was poured to ice water and extracted with ethyl acetate. The organic extract was washed with brine, dried over anhydrous sodium sulfate and concentrated to afford the pure title compound (5 g).

### Step 2

### 2,4-Dimethoxy-5-nitropyridine

A mixture of sodium methoxide (5.6 g) and 2,4-dichloro-5-nitropyridine (4 g) was heated to reflux overnight. The mixture was cooled to ambient temperature and methanol was removed under vacuum. Dichloromethane (150 mL) was added, washed with brine, dried over anhydrous sodium sulfate and concentrated. The residue was purified by column chromatography to afford the title compound (1.4 g).

### Step 3

### 4,6-Dimethoxypyridin-3-amine

A solution of 2,4-dimethoxy-5-nitropyridine (1.4 g) in ethyl acetate (80 mL) was added Pd/C (140 mg) and stirred at 1 atm of H₂ for 1.5 hours. Filtered and concentrated in vacuo to afford the pure title compound (1.1 g). MS m/z: 155 (MH⁺).

### Step 4

### 5-((4,6-Dimethoxypyridin-3-ylamino)methylene)-2,2-dimethyl-1,3-dioxane-4,6-dione

A mixture of 4,6-dimethoxypyridin-3-amine (1.4 g), 2,2-dimethyl-1,3-dioxane-4,6-dione (1.1 g) and trimethyl orthoformate (1.1 g) in ethanol (10 mL) were heated to reflux for overnight. The mixture was cooled to ambient temperature and filtered to afford the title compound as a white solid (2 g). MS m/z: 309 (MH⁺).

### Step 5

### 6,8-Dimethoxy-1,5-naphthyridin-4-ol

5-(4,6-Dimethoxypyridin-3-ylamino)methylene)-2,2-dimethyl-1,3-dioxane-4,6-dione (1 g) was added portionwise to diphenyl ether (5 mL) at 250° C. and stirred for 5 minutes.

The mixture was cooled to 50° C. and diluted with hexane. The resulting precipitates were collected by filtrate and washed with hexane to give the crude title compound (0.4 g). MS m/z: 207 (MH⁺).

### Step 6

### 8-Bromo-2,4-dimethoxy-1,5-naphthyridine

To a suspension of 6,8-dimethoxy-1,5-naphthyridin-4-ol (0.3 g) in anhydrous N,N-dimethylformamide (3 mL) was added phosphorous tribromide (0.6 g) under cooling with water, the mixture was stirred at room temperature for 2.5 hours. The mixture was poured into ice water (10 mL), and the mixture was adjusted to pH 8 by addition of saturated sodium hydrogencarbonate solution. The resulting precipitates were collected by filtrate, washed with water, and dried. Flash chromatography of the crude product gave the title compound (0.3 g). MS m/z: 269 (MH⁺).

### Step 7

### tert-Butyl 1-(2-(6,8-Dimethoxy-1,5-naphthyridin-4-yl)ethyl)-2-oxabicyclo[2.2.2]octan-4-ylcarbamate

To a solution of B (100 mg) in anhydrous tetrahydrofuran (1.8 mL) was added a solution of 9-borabicyclo[3.3.1]nonane dimer (1.6 mL, 0.5 M in tetrahydrofuran) under cooling with ice, the mixture was stirred at room temperature for 1 hour. After quenching the reaction by adding water (1 drop) under cooling, the mixture was added 8-bromo-2,4-dimethoxy-1,5-naphthyridine (114.4 mg), tetrakis(triphenylphosphine)palladium (91.2 mg), tripotassium phosphate (0.6 g) and ethanol/water (1 mL, 4:1), and degassed. The mixture was heated at 70° C. for 12 hours and concentrated in vacuo. After dilution of the residue with ethyl acetate, the mixture was washed with water and brine, dried over anhydrous sodium sulfate, filtered, and then concentrated in vacuo gave the crude title compound, which was used directly.

### Step 8

### 1-(2-(6,8-Dimethoxy-1,5-naphthyridin-4-yl)ethyl)-2-oxabicyclo[2.2.2]octan-4-amine

To a solution of 1-(2-(6,8-dimethoxy-1,5-naphthyridin-4-yl)ethyl)-2-oxabicyclo[2.2.2]octan-4-ylcarbamate (0.2 g crude) in dichloromethane (4 mL) was added trifluoroacetic acid (4 mL) and the mixture was stirred at room temperature for 30 minutes and concentrated in vacuo. After dilution of the residue with water, the mixture was washed with methyl tert-butyl ether twice. The aqueous layer was adjusted to pH 13 by addition of aqueous sodium carbonate solution and extract twice with ethyl acetate. The combined ethyl acetate layer was washed with brine, dried over anhydrous sodium sulfate and condensed to give the pure title compound. MS m/z: 344 (MH⁺).

### Step 9

### 6-((1-(2-(6,8-Dimethoxy-1,5-naphthyridin-4-yl)ethyl)-2-oxabicyclo[2.2.2]octan-4-ylamino)methyl)-2H-pyrido[3,2-b][1,4]oxazin-3(4H)-one

A mixture of 1-(2-(6,8-dimethoxy-1,5-naphthyridin-4-yl)ethyl)-2-oxabicyclo[2.2.2]octan-4-amine (101 mg) and I (80.1 mg) in anhydrous N,N-dimethylformamide (3.5 mL) was added acetic acid (0.5 mL) was stirred at room temperature for 30 minutes. The resulting solution was added three times of sodium triacetoxyborohydride (127 mg) and stirred at room temperature overnight. The mixture was concentrated in vacuo. After dilution of the residue with dichloromethane, the mixture was washed with saturated sodium carbonate solution, water and brine. The organic extracts were dried over anhydrous sodium sulfate then concentrated in vacuo. The residue was purified by prep-TLC (dichloromethane:methanol=10:1) to give (70 mg). To a solution of the free base (70 mg) in dichloromethane (2 mL) and ethanol (0.5 mL) was added a solution of hydrogen chloride (33 uL, 4 M in 1,4-dioxane) under cooling with ice, the mixture was stirred at room temperature for 2 hours and concentrated in vacuo. Treatment of the residue with ethanol gave the title compound as its HCl salt.

¹H NMR (CD₃OD): δ 1.92-1.99 (m, 4H), 2.13-2.14 (m, 6H), 3.42-3.46 (m, 2H), 4.00 (s, 2H), 4.13 (s, 3H), 4.22 (s, 5H), 4.67 (s, 2H), 7.04 (s, 1H), 7.12 (d, J=8.0 Hz, 1H), 7.35 (d, J=8.0 Hz, 1H), 8.12 (d, J=5.6 Hz, 1H), 8.77 (d, J=5.6 Hz, 1H).

MS m/z: 506 (MH⁺).

### Example 151

### 6-((1-(2-(3-Fluoro-6-methoxy-1,5-naphthyridin-4-yl)-1-hydroxypropyl)-2-oxabicyclo[2.2.2]octan-4-ylamino)methyl)-2H-pyrido[3,2-b][1,4]oxazin-3(4H)-one

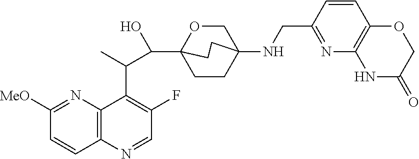 [SMILES: COc1ccc2ncc(F)c(C(C)C(O)C34CCC(NCc5ccc6c(n5)NC(=O)CO6)(CC3)CO4)c2n1]

### Step 1

### tert-Butyl 1-(2-(3-Fluoro-6-methoxy-1,5-naphthyridin-4-yl)acetyl)-2-oxabicyclo[2.2.2]octan-4-ylcarbamate

A suspension of tert-butyl {1-[2-(3-fluoro-6-methoxy-1,5-naphthyridin-4-yl)-1-hydroxyethyl]-2-oxabicyclo[2.2.2]oct-4-yl}carbamate (1.4 g, (+)-form) and Dess-Martin periodinane (2.0 g) was stirred at room temperature for 4 hours. Filtrated and the solid was washed with dichloromethane. The filtrate was condensed and the residue was purified by prep-TLC (petroleum ether:ethyl acetate=3:1) to afford a white solid (1.39 g).

¹H NMR (CDCl₃): δ 1.42 (s, 9H), 1.90-1.98 (m, 2H), 2.04-2.11 (m, 6H), 3.98 (s, 3H), 4.13 (s, 2H), 4.40 (brs, 1H), 4.52 (s, 2H), 7.03 (d, J=9.2 Hz, 1H), 8.16 (d, J=9.2 Hz, 1H), 8.62 (s, 1H).

### Step 2

### tert-Butyl 1-(2-(3-Fluoro-6-methoxy-1,5-naphthyridin-4-yl)propanoyl)-2-oxabicyclo[2.2.2]octan-4-ylcarbamate

A solution of tert-butyl 1-(2-(3-fluoro-6-methoxy-1,5-naphthyridin-4-yl)acetyl)-2-oxabicyclo[2.2.2]octan-4-ylcarbamate (223 mg) in dry tetrahydrofuran (10 mL) was added lithium bis(trimethylsilyl)amide (1 mL) dropwise at −78° C. and stirred for 30 minutes. Then iodomethane (213 mg) was added slowly by a syringe. The mixture was stirred at −78° C. for 30 minutes then warmed to room temperature and stirred overnight. Quenching the reaction by adding saturated ammonium chloride solution and extracted with ethyl acetate twice. The organic layer was condensed and the residue was purified by prep-TLC (petroleum ether:ethyl acetate=3:1) to afford the title compound as a white solid (171 mg).

¹H NMR (CDCl₃): δ 1.34 (s, 9H), 1.44 (d, J=6.8 Hz, 1H), 1.69-1.77 (m, 2H), 1.84-1.88 (m, 2H), 1.94-1.96 (m, 4H), 3.49-3.52 (brs, 1H), 3.69-3.72 (m, 1H), 3.98 (s, 3H), 4.09 (q, J=7.2 Hz, 1H), 4.24 (s, 1H), 4.81 (q, J=6.8 Hz, 1H), 7.02 (d, J=9.2 Hz, 1H), 8.14 (d, J=9.2 Hz, 1H), 8.61 (s, 1H).

MS m/z: 460 (MH⁺).

### Step 3

### tert-Butyl 1-(2-(3-Fluoro-6-methoxy-1,5-naphthyridin-4-yl)-1-hydroxypropyl)-2-oxabicyclo[2.2.2]octan-4-ylcarbamate

A solution of tert-butyl 1-(2-(3-fluoro-6-methoxy-1,5-naphthyridin-4-yl)propanoyl)-2-oxabicyclo[2.2.2]octan-4-ylcarbamate (171 mg) in methanol (10 mL) was added sodium borohydride (38 mg) at 0° C. and stirred overnight. The mixture was diluted with ethyl acetate (50 mL) and washed with water twice. The organic layer was condensed and the residue was purified by prep-TLC (petroleum ether:ethyl acetate=2:1) to afford a white solid (124 mg).

MS m/z: 462 (MH⁺).

### Step 4

### 1-(4-Amino-2-oxabicyclo[2.2.2]octan-1-yl)-2-(3-fluoro-6-methoxy-1,5-naphthyridin-4-yl)propan-1-ol

To a solution of tert-butyl 1-(2-(3-fluoro-6-methoxy-1,5-naphthyridin-4-yl)-1-hydroxypropyl)-2-oxabicyclo[2.2.2]octan-4-ylcarbamate (124 mg) in dichloromethane (1 mL) was added trifluoroacetic acid (1 mL) and the mixture was stirred at room temperature for 30 minutes and concentrated in vacuo. After dilution of the residue with water, the mixture was washed with methyl tert-butyl ether twice. The aqueous layer was adjusted to pH 13 by addition of aqueous sodium carbonate solution and extracted twice with ethyl acetate. The combined ethyl acetate layer was washed with brine, dried over anhydrous sodium sulfate and condensed to give the pure title compound (73 mg). MS m/z: 362 (MH⁺).

### Step 5

### 6-((1-(2-(3-Fluoro-6-methoxy-1,5-naphthyridin-4-yl)-1-hydroxypropyl)-2-oxabicyclo[2.2.2]octan-4-ylamino)methyl)-2H-pyrido[3,2-b][1,4]oxazin-3(4H)-one

A mixture of 1-(4-amino-2-oxabicyclo[2.2.2]octan-1-yl)-2-(3-fluoro-6-methoxy-1,5-naphthyridin-4-yl)propan-1-ol (73 mg) and I (53 mg) in anhydrous N,N-dimethylformamide (3.5 mL) was added acetic acid (0.5 mL) was stirred at room temperature for 30 minutes. The resulting solution was added three times of sodium triacetoxyborohydride (64 mg) and stirred at room temperature overnight. The mixture was concentrated in vacuo. After dilution of the residue with dichloromethane, the mixture was washed with saturated sodium carbonate solution, water and brine. The organic extracts were dried over anhydrous sodium sulfate then concentrated in vacuo. The residue was purified by prep-TLC (dichloromethane:methanol=10:1) to afford a solid. To a solution of this solid (32 mg) in dichloromethane (2 mL) and ethanol (0.5 mL) was added a solution of hydrogen chloride (15 uL, 4 M in 1,4-dioxane) under cooling with ice, the mixture was stirred at room temperature for 2 hours and concentrated in vacuo. This white solid racemic mixture was separated using SFC (supercritical fluid chromatography) to give two isomers.

The first eluted isomer: ¹H NMR (MeOD): δ 1.41-2.11 (m, 11H), 3.43-4.01 (m, 2H), 4.09 (s, 3H), 4.60 (s, 2H), 6.94 (d, J=8.0 Hz, 1H), 7.15 (d, J=9.2 Hz, 1H), 7.23 (d, J=8.0 Hz, 1H), 8.19 (d, J=9.2 Hz, 1H), 8.60 (s, 1H).

MS m/z: 524 (MH⁺).

The second eluted isomer: ¹H NMR (MeOD): δ 1.41-2.17 (m, 11H), 3.43-4.02 (m, 2H), 4.09 (s, 3H), 4.60 (s, 2H), 6.93 (d, J=8.0 Hz, 1H), 7.15 (d, J=9.2 Hz, 1H), 7.23 (d, J=8.0 Hz, 1H), 8.19 (d, J=9.2 Hz, 1H), 8.60 (s, 1H).

MS m/z: 524 (MH⁺).

### Example 152

### 6-((1-(2-(3-Fluoro-6-methyl-1,5-naphthyridin-4-yl)ethyl)-2-oxabicyclo[2.2.2]octan-4-ylamino)methyl)-2H-pyrido[3,2-b][1,4]oxazin-3(4H)-one

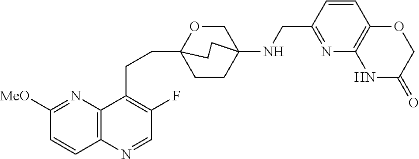 [SMILES: COc1ccc2ncc(F)c(CCC34CCC(NCc5ccc6c(n5)NC(=O)CO6)(CC3)CO4)c2n1]

### Step 1

### 2-(1-Ethoxyvinyl)-6-methyl-3-nitropyridine

Tributyl(1-ethoxyvinyl)tin (25 g) was added into the mixture of 2-chloro-6-methyl-3-nitropyridine (10 g) and bis(triphenylphosphine)palladium(II) dichloride (1.1 g) in acetonitrile (50 mL) at 65° C. The resulting suspension was stirred at 65° C. for 4 hours then cooled to room temperature. The reaction mixture was quenched with 10% potassium fluoride aqueous solution (50 mL) and stirred at room temperature for 1 hour. Then the mixture was filtered, and the filtrate was extracted with ethyl acetate. The combined organic phases were dried over sodium sulfate, concentrated and purified by column chromatography to give the title compound (10.3 g).

¹H NMR (CDCl₃): δ 1.29 (t, J=3.2 Hz, 3H), 2.64 (s, 3H), 3.86-3.91 (m, 2H), 4.52 (s, 1H), 4.99 (s, 1H), 7.22 (d, J=8.4 Hz, 1H), 7.90 (d, J=8.0 Hz, 1H).

### Step 2

### 2-Fluoro-1-(6-methyl-3-nitropyridin-2-yl)ethanone

To a suspension of SELECTFLUOR (Fisher Scientific, Pittsburg, Pa.) (7.6 g) in acetonitrile (20 mL) and water (10 mL) was added dropwise a solution of 2-(1-ethoxyvinyl)-6-methyl-3-nitropyridine (3 g) in acetonitrile (10 mL) over 15 minutes, the resulting mixture was stirred at room temperature for 4 hours. Then water was added, and the mixture was extracted with ethyl acetate. The combined organic phases were dried over sodium sulfate, and concentrated to give the title compound (2.4 g).

¹H NMR (CDCl₃): δ 2.70 (s, 3H), 5.37 (s, 1H), 5.49 (s, 1H), 7.46 (d, J=8.4 Hz, 1H), 8.28 (d, J=8.4 Hz, 1H).

### Step 3

### (Z)-3-(Dimethylamino)-2-fluoro-1-(6-methyl-3-nitropyridin-2-yl)prop-2-en-1-one

To a solution of 2-fluoro-1-(6-methyl-3-nitropyridin-2-yl)ethanone (2 g) in N,N-dimethylformamide (15 mL) was added N,N-dimethylformamide-N,N-dimethylacetamide (10 mL). The reaction mixture was heated to 50° C. and stirred for 4 hours under nitrogen. Then the mixture was cooled to room temperature and filtered to give the title compound (2.3 g) as a yellow solid. It was used in next step directly.

### Step 4

### 3-Fluoro-6-methyl-1,5-naphthyridin-4-ol

A solution of (Z)-3-(dimethylamino)-2-fluoro-1-(6-methyl-3-nitropyridin-2-yl)prop-2-en-1-one (1 g) and ammonium chloride (1.06 g) in methanol/water (1:1, 30 mL) was cooled to 0° C., then iron dust (2.21 g) was added in portions, and then slowly warmed to 65° C. for 5 hours. When the reaction was completed, it was cooled to room temperature and filtered. The filtrate was washed with ethyl acetate. The combined extracts were washed with brine, dried over sodium sulfate and concentrated to give the title compound (400 mg).

### Step 5

### 8-Bromo-7-fluoro-2-methyl-1,5-naphthyridine

To a suspension of 3-fluoro-6-methyl-1,5-naphthyridin-4-ol (400 mg) in anhydrous N,N-dimethylformamide (8 mL) was added phosphorous tribromide (0.5 mL) under 0° C. The mixture was stirred at room temperature for 2.5 hours. The mixture was poured into ice water (20 mL), the mixture was adjusted to pH 8 by addition of saturated sodium bicarbonate solution. The resulting precipitates were collected by filtration, washed with water and dried. The crude product was purified by prep-TLC (toluene:ethyl acetate=5:1) to give the title compound (300 mg).

¹H NMR (CDCl₃): δ 2.80 (s, 3H), 7.49 (d, J=8.4 Hz, 1H), 8.26 (d, J=8.4 Hz, 1H), 8.70 (s, 1H).

### Step 6

### tert-Butyl 1-(2-(3-Fluoro-6-methyl-1,5-naphthyridin-4-yl)ethyl)-2-oxabicyclo[2.2.2]octan-4-ylcarbamate

B (100 mg) in tetrahydrofuran (2 mL) was stirred under ice-bath. Then 9-borabicyclo(3.3.1)nonane dimer (1.6 mL) was added slowly. The mixture was stirred at room temperature for 2 hours. Then water (0.5 mL) was added. To the mixture was added 8-bromo-7-fluoro-2-methyl-1,5-naphthyridine (96 mg), tripotassium phosphate (169 mg), tetrakis(triphenylphosphine)palladium (10 mg) and ethanol (3 mL). The resulting mixture was stirred at 80° C. overnight. The mixture was filtered and the crude compound was used in the next step directly.

### Step 7

### 1-(2-(3-Fluoro-6-methyl-1,5-naphthyridin-4-yl)ethyl)-2-oxabicyclo[2.2.2]octan-4-amine

### tert-Butyl 1-(2-(3-fluoro-6-methyl-1,5-naphthyridin-4-yl)ethyl)-2-oxabicyclo[2.2.2]octan-4-ylcarbamate (100 mg, crude) in dichloromethane/trifluoroacetic acid (3 mL3:1) was stirred at room temperature for 1 hour. Then the mixture was concentrated to give the title compound. It was used in the next step directly.

### Step 8

### 6-((1-(2-(3-Fluoro-6-methyl-1,5-naphthyridin-4-yl)ethyl)-2-oxabicyclo[2.2.2]octan-4-ylamino)methyl)-2H-pyrido[3,2-b][1,4]oxazin-3(4H)-one

A mixture of 1-(2-(3-fluoro-6-methyl-1,5-naphthyridin-4-yl)ethyl)-2-oxabicyclo[2.2.2]octan-4-amine (50 mg), I (34 mg) acetic acid (0.1 mL) and N,N-dimethylformamide (3 mL) was stirred at room temperature for 2 hours. Then sodium triacetoxyborohydride (203 mg) was added into the mixture. The resulting mixture was stirred at room temperature for another 12 hours. Then the mixture was pushed into water and adjusted to pH 8-9 with aq. sodium hydrogencarbonate. Then the mixture was extracted with ethyl acetate. The combined organic phases were washed with brine, dried over sodium sulfate, filtered and concentrated. The crude compound was purified by prep-HPLC to give the title compound.

¹H NMR (CD₃OD): δ 1.84-1.88 (m, 2H), 1.92-2.25 (m, 8H), 2.75 (s, 3H), 3.35-3.38 (m, 2H), 3.95 (s, 2H), 4.20 (s, 2H), 4.68 (s, 2H), 7.08 (d, J=8.8 Hz, 1H), 7.36 (d, J=8.0 Hz, 1H), 7.59 (d, J=8.8 Hz, 1H), 8.23 (d, J=8.8 Hz, 1H), 8.74 (s, 1H).

### Example 153

### 6-((1-(2-(7-Fluoro-2-methoxyquinolin-8-yl)ethyl)-2-oxabicyclo[2.2.2]octan-4-ylamino)methyl)-2H-pyrido[3,2-b][1,4]oxazin-3(4H)-one

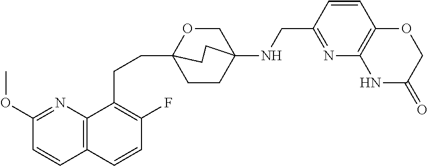 [SMILES: COc1ccc2ccc(F)c(CCC34CCC(NCc5ccc6c(n5)NC(=O)CO6)(CC3)CO4)c2n1]

### Step 1

### N-(2-Bromo-3-fluorophenyl)-3-phenylacrylamide

To a mixture of 2-bromo-3-fluoroaniline (10 g) and potassium carbonate (14.7 g) in acetone (50 mL) and water (10 mL) was added dropwise over 15 minutes cinnamoyl chloride (10.6 g) in acetone (5 mL). And the resulting mixture was stirred at room temperature for 4 hours. Then water was added, and the mixture was filtered to give the title compound (5 g) as a white solid.

¹H NMR (CDCl₃): δ 6.53 (d, J=15.6 Hz, 1H), 6.85 (s, 1H), 7.25-7.34 (m, 4H), 7.52 (s, 2H), 7.73 (d, J=16.4 Hz, 2H), 8.29 (d, J=6.8 Hz, 1H).

### Step 2

### 8-Bromo-7-fluoroquinolin-2(1H)-one

To a solution of N-(2-bromo-3-fluorophenyl)-3-phenylacrylamide (5 g) in chlorobenzene (50 mL) was added aluminum chloride (10.2 g). The mixture was stirred at 80° C. for 3 hours. Then the mixture was poured into ice water and filtered. The filtrate was extracted with ethyl acetate. The combined organic phases were dried over sodium sulfate, concentrated and purified by column chromatography to give the title compound (3 g).

¹H NMR (CDCl₃): δ 6.55 (d, J=9.6 Hz, 1H), 6.97 (t, J=8.0 Hz, 1H), 7.43-7.47 (m, 1H), 7.63 (d, J=9.6 Hz, 1H), 8.99 (s, 1H).

### Step 3

### 8-Bromo-2-chloro-7-fluoroquinoline

To a solution of 8-bromo-7-fluoroquinolin-2(1H)-one (3 g) in N,N-dimethylformamide (20 mL) was added phosphorous oxychloride (6 mL) at 0° C. The mixture was stirred at 80° C. for 3 h. Then the mixture was poured into ice water and filtered to give the title compound (2 g).

¹H NMR (CDCl₃): δ 7.32-7.37 (m, 2H), 7.71-7.74 (m, 1H), 8.05 (d, J=8.4 Hz, 1H).

### Step 4

### 8-Bromo-7-fluoro-2-methoxyquinoline

To a suspension of 8-bromo-2-chloro-7-fluoroquinoline (1 g) in methanol (10 mL) was added sodium methoxide (209 mg) dropwise in methanol (2 mL) over 5 minutes and the resulting mixture was stirred at 60° C. for 12 hours. Then water was added, and the mixture was extracted with ethyl acetate. The combined organic phases were dried over sodium sulfate, concentrated and purified by column chromatography to give the title compound (500 mg).

¹H NMR (CDCl₃): δ 4.08 (s, 3H), 6.83 (d, J=8.8 Hz, 1H), 7.13 (t, J=8.4 Hz, 1H), 7.56-7.60 (m, 1H), 7.88 (d, J=8.8 Hz, 1H).

### Step 5

### tert-Butyl 1-(2-(7-Fluoro-2-methoxyquinolin-8-yl)ethyl)-2-oxabicyclo[2.2.2]octan-4-ylcarbamate

To a mixture of B (100 mg) in tetrahydrofuran (2 mL) was added 9-borabicyclo(3.3.1)nonane dimer (1.7 mL) at 0° C., and the resulting mixture was stirred at room temperature for 2 hours. Then the reaction mixture was quenched with water (2 mL). To the mixture was added tripotassium phosphate (169 mg), 8-bromo-7-fluoro-2-methoxyquinoline (102 mg), ethanol (2 mL) and tetrakis(triphenylphosphine)palladium (120 mg) at room temperature. Then the resulting mixture was stirred at 90° C. for 12 hours. Then water was added, and the mixture was extracted with ethyl acetate. The combined organic phases were dried over sodium sulfate, and concentrated to give the crude title compound (150 mg). The crude was used in the next step directly.

### Step 6

### 1-(2-(7-Fluoro-2-methoxyquinolin-8-yl)ethyl)-2-oxabicyclo[2.2.2]octan-4-amine

To a mixture of tert-butyl 1-(2-(7-fluoro-2-methoxyquinolin-8-yl)ethyl)-2-oxabicyclo[2.2.2]octan-4-ylcarbamate (150 mg) in dichloromethane (5 mL) was added trifluoroacetic acid (2 mL) and the resulting mixture was stirred at room temperature for 2 hours. Then water was added, and the mixture was extracted with ethyl acetate. The combined organic phases were dried over sodium sulfate, and concentrated to give the crude title compound (100 mg). The crude was used in the next step directly.

### Step 7

### 6-((1-(2-(7-Fluoro-2-methoxyquinolin-8-yl)ethyl)-2-oxabicyclo[2.2.2]octan-4-ylamino)methyl)-2H-pyrido[3,2-b][1,4]oxazin-3(4H)-one

To a mixture of 1-(2-(7-fluoro-2-methoxyquinolin-8-yl)ethyl)-2-oxabicyclo[2.2.2]octan-4-amine (100 mg) and I (53 mg) in methanol (5 mL) was added acetic acid (0.1 mL) and the resulting mixture was stirred at room temperature for 12 hours. Then sodium triacetoxyborohydride (70 mg) was added, and the mixture was stirred at room temperature for 2 hours. The reaction mixture was quenched water (10 mL) and the mixture was extracted with ethyl acetate. The combined organic phases were dried over sodium sulfate, concentrated and purified by prep-HPLC to give the title compound (10 mg).

¹H NMR (CD₃OD): δ 1.67-1.72 (m, 2H), 1.91-2.05 (m, 8H), 3.11 (t, J=7.6 Hz, 2H), 3.93 (s, 2H), 3.97 (s, 3H), 4.14 (s, 2H), 4.61 (s, 2H), 6.79 (d, J=8.8 Hz, 1H), 7.01-7.09 (m, 2H), 7.28 (t, J=8.4 Hz, 1H), 7.55-7.59 (m, 1H), 7.98 (d, J=9.2 Hz, 1H).

### Example 154

### 6-((1-(2-(6-(Dimethylamino)-7-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

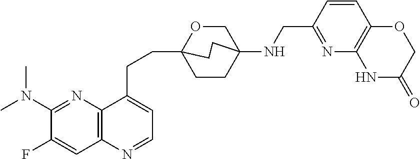 [SMILES: CN(C)c1nc2c(CCC34CCC(NCc5ccc6c(n5)NC(=O)CO6)(CC3)CO4)ccnc2cc1F]

### Step 1

### 2-Hydroxy-5-nitronicotinic Acid

To a solution of 2-hydroxypyridine-3-carboxylic acid (50 g) in concentrated sulfuric acid (500 mL) at 0° C. was added fuming nitric acid (45 mL) dropwise. The mixture was stirred at the same temperature for 30 minutes, and stirred at 50-60° C. for another 2 hours. It was poured into ice and filtered. The precipitates were washed with water, then it was dried with high vacuo, it was used in the next step without further purification (55 g).

¹H NMR (DMSO-d₆): δ 8.67 (d, J=3.2 Hz, 1H), 8.97 (d, J=3.2 Hz, 1H).

### Step 2

### 2-Chloro-5-nitronicotinic Acid

A suspension of the 2-hydroxy-5-nitronicotinic acid (20 g) in phosphorous oxychloride (80 mL) was heated at 100° C. for 1.5 hours. The reaction mixture was allowed to cool to room temperature, and then poured into ice. The resulting precipitate was collected by filtration. The filtrate was concentrated in vacuo to afford the crude title compound (18 g).

¹H NMR (DMSO-d₆): δ 8.88 (d, J=2.8 Hz, 1H), 9.35 (d, J=2.8 Hz, 1H).

### Step 3

### 2-(Dimethylamino)-5-nitronicotinic Acid

The mixture of the 2-chloro-5-nitronicotinic acid (8 g), dimethylamine hydrochloride (3.48 g) and potassium carbonate (11 g) in acetonitrile (80 mL) were refluxed for 8 hours. After dilution of the mixture with ethyl acetate (100 mL), solid was filtered off. The filtrate was washed with citric acid and brine. The organic extracts were dried with sodium sulfate, concentrated in vacuo to give the title compound, which was used in the next step without further purification (8 g).

¹H NMR (DMSO-d₆): δ 3.15 (s, 6H), 8.35 (d, J=2.4 Hz, 1H), 8.94 (d, J=2.8 Hz, 1H).

### Step 4

### tert-Butyl 2-(Dimethylamino)-5-nitropyridin-3-ylcarbamate

A mixture of 2-(dimethylamino)-5-nitronicotinic acid (2.5 g), diphenyl phosphoryl azide (4 mL), triethylamine (2.5 mL) and anhydrous tert-butanol (15 mL) were heated at 100° C. for 1 hour and concentrated in vacuo. After dilution of the residue with ethyl acetate, the mixture was washed with saturated sodium bicarbonate solution and brine. The organic phases were dried over anhydrous sodium sulfate, filtered, and then concentrated in vacuo. It was purified by column chromatography (hexane:ethyl acetate=5:1) to give the title compound (2.5 g).

¹H NMR (CDCl₃): δ 1.52 (s, 9H), 3.15 (s, 6H), 8.83 (d, J=2.4 Hz, 2H).

### Step 5

### N,N-Dimethyl-5-nitropyridine-2,3-diamine

To a solution of tert-butyl 2-(dimethylamino)-5-nitropyridin-3-ylcarbamate (500 mg) in dichloromethane (5 mL) was added trifluoroacetic acid (2 mL) at −10° C., the mixture was stirred at room temperature overnight and concentrated in vacuo. Diluted with ethyl acetate, the organic extracts were dried over anhydrous sodium sulfate, filtered, and then concentrated in vacuo to give the title compound (250 mg).

¹H NMR (CDCl₃): δ 2.89 (s, 6H), 7.54 (d, J=2.4 Hz, 1H), 8.59 (d, J=2.4 Hz, 1H).

### Step 6

### 3-Fluoro-N,N-dimethyl-5-nitropyridin-2-amine

To a solution of N,N-dimethyl-5-nitropyridine-2,3-diamine (250 mg) in anhydrous tetrahydrofuran (5 mL) was added nitrosyl tetrafluoroborate (165 mg) at −10° C., the mixture was stirred at the same temperature for 50 minutes. Another nitrosyl tetrafluoroborate (16.5 g) was added to the mixture at the same temperature. After stirred for 5 minutes, the resulting precipitates were collected by filtration and washed with cold tetrahydrofuran to give diazonium salt as yellow solid (240 mg). A suspension of the salt (240 mg) in decaline (2 mL) was heated at 100° C. for 30 minutes. After cooling with NaCl-ice bath, the precipitates were collected by filtration and dissolved with ethyl acetate. The mixture was washed with saturated sodium bicarbonate solution, dried over anhydrous sodium sulfate, filtered, and then concentrated in vacuo. It was purified by column chromatography (toluene:ethyl acetate=30:1) to give the title compound (100 mg).

¹H NMR (CDCl₃): δ 3.24 (s, 6H), 7.86 (m, 1H), 8.79 (t, J=1.6 Hz, 1H).

### Step 7

### 3-Fluoro-N,N-dimethylpyridine-2,5-diamine

To a degassed solution of 3-fluoro-N,N-dimethyl-5-nitropyridin-2-amine (100 mg) in methanol (5 mL) was added Raney-Ni (10 mg), the mixture was stirred at room temperature for 2 hours under H₂ (5 psi). After filtering and concentration, the resulting precipitates were collected to give the crude title compound (60 mg).

¹H NMR (CDCl₃): δ 2.84 (s, 6H), 6.70 (m, 1H), 7.50 (d, J=1.2 Hz, 1H).

### Step 8

### 5-((6-(Dimethylamino)-5-fluoropyridin-3-ylamino)methylene)-2,2-dimethyl-1,3-dioxane-4,6-dione

A mixture of 3-fluoro-N,N-dimethylpyridine-2,5-diamine (150 mg), Meldrum's acid (144 mg) and triethyl orthoformate (0.5 mL) in ethanol (5 mL) was refluxed for 1 hour. The resulting precipitates were collected by filtration and washed with ethanol to give the title compound (100 mg).

¹H NMR (CDCl₃): δ 1.64 (s, 6H), 3.22 (s, 6H), 7.35 (m, 1H), 7.98 (s, 1H), 8.51 (s, 1H).

### Step 9

### 6-(Dimethylamino)-7-fluoro-1,5-naphthyridin-4-ol

5-((6-(Dimethylamino)-5-fluoropyridin-3-ylamino)methylene)-2,2-dimethyl-1,3-dioxane-4,6-dione (50 mg) was added in several portions to diphenyl ether (2 mL) at 260° C. over 5 minutes. After cooled, the mixture was diluted with petroleum ether. The resulting precipitates were collected by filtration and washed with petroleum ether to give the crude title compound (20 mg).

¹H NMR (CDCl₃): δ 3.25 (s, 6H), 6.85 (s, 1H), 7.74 (d, J=14.4 Hz, 1H), 8.41 (t, J=2.0 Hz, 1H).

### Step 10

### 8-Bromo-3-fluoro-N,N-dimethyl-1,5-naphthyridin-2-amine

To a suspension of 6-(dimethylamino)-7-fluoro-1,5-naphthyridin-4-ol (400 mg) in anhydrous N,N-dimethylformamide (2 mL) was added phosphorous tribromide (0.5 mL) at 0° C., the mixture was stirred at room temperature for 2.5 hours. The mixture was poured into ice water (20 mL), the mixture was adjusted to pH 8 by addition of saturated sodium bicarbonate solution. The resulting precipitates were collected by filtration, washed with water and dried. It was purified by prep-TLC (toluene:ethyl acetate=5:1) to give the title compound (200 mg).

¹H NMR (CDCl₃): δ 3.25 (s, 6H), 7.73 (m, 2H), 8.34 (d, J=4.4 Hz, 1H)

### Step 11

### tert-Butyl 1-(2-(6-(Dimethylamino)-7-fluoro-1,5-naphthyridin-4-yl)ethyl)-2-oxabicyclo[2.2.2]octan-4-ylcarbamate

B (100 mg) in tetrahydrofuran (2 mL) was stirred under ice-bath. Then 9-borabicyclo(3.3.1)nonane dimer (1.6 mL) was added slowly. The mixture was stirred at room temperature for 2 hours. Then water (0.5 mL) was added. To the mixture was added 8-bromo-3-fluoro-N,N-dimethyl-1,5-naphthyridin-2-amine (108 mg), tripotassium phosphate (169 mg), tetrakis(triphenylphosphine)palladium (10 mg) and ethanol (3 mL). The resulting mixture was stirred at 80° C. overnight. The mixture was filtered and the crude compound was used in the next step directly.

### Step 12

### 8-(2-(4-Amino-2-oxabicyclo[2.2.2]octan-1-yl)ethyl)-3-fluoro-N,N-dimethyl-1,5-naphthyridin-2-amine

### tert-Butyl 1-(2-(6-(dimethylamino)-7-fluoro-1,5-naphthyridin-4-yl)ethyl)-2-oxabicyclo[2.2.2]octan-4-ylcarbamate (100 mg) in dichloromethane/trifluoroacetic acid (4 mL3:1) was stirred at room temperature for 1 hour. Then the mixture was concentrated to give the title compound.

### Step 13

### 6-((1-(2-(6-(Dimethylamino)-7-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

A mixture of 8-(2-(4-amino-2-oxabicyclo[2.2.2]octan-1-yl)ethyl)-3-fluoro-N,N-dimethyl-1,5-naphthyridin-2-amine (80 mg, crude), I (30 mg), acetic acid (0.1 mL) and N,N-dimethylformamide (3 mL) was stirred at room temperature for 2 hours. Then sodium triacetoxyborohydride (250 mg) was added into the mixture. The resulting mixture was stirred at room temperature for another 12 hours. Then the mixture was pushed into water and basified to pH 8-9 with aq. sodium hydrogencarbonate. Then the mixture was extracted with ethyl acetate. The combined organic phases were washed with brine, dried over sodium sulfate, filtered and concentrated. The crude compound was purified by prep-HPLC to give the title compound.

¹H NMR (CD₃OD): δ 1.86-1.96 (m, 4H), 2.07-2.15 (m, 6H), 3.28-3.30 (m, 8H), 3.99 (s, 2H), 4.20 (s, 2H), 4.68 (s, 2H), 7.08 (d, J=8.4 Hz, 1H), 7.34 (d, J=8.4 Hz, 1H), 7.81 (d, J=5.6 Hz, 1H), 7.89 (d, J=13.2 Hz, 1H), 8.59 (d, J=5.6 Hz, 1H).

### Example 155

### 6-((1-(2-(3-Fluoro-6-methoxy-1,5-naphthyridin-4-yl)ethyl)-2-oxabicyclo[2.2.2]octan-4-ylamino)methyl)-2H-pyrido[3,2-b][1,4]thiazin-3(4H)-one

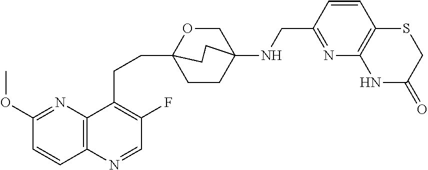 [SMILES: COc1ccc2ncc(F)c(CCC34CCC(NCc5ccc6c(n5)NC(=O)CS6)(CC3)CO4)c2n1]

### Step 1

### Methyl 6-Aminopicolinate

To a solution of 6-aminopyridine-2-carboxylic acid (10 g) in methanol (150 mL) was added concentrated sulfuric acid (20 mL) dropwise. The mixture was refluxed for 16 hours. Most of the methanol was removed in vacuo. The residue was poured into ice-water. The mixture was adjusted to pH 8-9 with 6 N sodium hydroxide solution and then extracted with ethyl acetate. The organic phases were washed with brine, dried over sodium sulfate, filtered. The filtrate was concentrated to give the title compound (7.5 g).

¹H NMR (CDCl₃): δ 3.91 (s, 3H), 4.91 (br, 2H), 6.65 (d, J=8.0 Hz, 1H), 7.45 (d, J=7.2 Hz, 1H), 7.50 (t, J=7.6 Hz, 1H).

### Step 2

### Methyl 6-Amino-5-bromopicolinate

To a solution of methyl 6-aminopicolinate (2.0 g) in chloroform (60 mL) was added a solution of bromine (2.1 g) in chloroform (10 mL) at room temperature. The mixture was stirred overnight at room temperature. The mixture was washed with saturated sodium hydrogencarbonate, extracted with dichloromethane. The organic phases were washed with brine, dried over sodium sulfate, filtered and concentrated. The residue was purified by column chromatography on silica gel to give the title compound (0.5 g).

¹H NMR (CDCl₃): δ 3.88 (s, 3H), 5.34 (br, 2H), 7.28 (d, J=8.0 Hz, 1H), 7.71 (d, J=7.6 Hz, 1H).

### Step 3

### Methyl 3-Oxo-3,4-dihydro-2H-pyrido[3,2-b][1,4]thiazine-6-carboxylate

To a solution of methyl 6-amino-5-bromopicolinate (2.3 g) in N,N-dimethylformamide (40 mL) was added sodium hydride (0.48 g, 60% in mineral oil) at 0° C. Then ethyl sulfanylacetate (1.2 g) was added. The mixture was stirred overnight at room temperature. The mixture was poured into ice-water and the resultant mixture was extracted with ethyl acetate. The organic phases were washed with brine, dried over sodium sulfate, filtered. The filtrate was concentrated to give the title compound (0.6 g).

¹H NMR (DMSO-d₆): δ 3.61 (s, 2H), 3.83 (s, 3H), 7.64 (d, J=8.0 Hz, 1H), 7.95 (d, J=8.0 Hz, 1H), 11.26 (s, 1H).

### Step 4

### 6-(Hydroxymethyl)-2H-pyrido[3,2-b][1,4]thiazin-3(4H)-one

To a solution of methyl 3-oxo-3,4-dihydro-2H-pyrido[3,2-b][1,4]thiazine-6-carboxylate (0.5 g) in tetrahydrofuran (15 mL) was added a solution of diisobutylaluminum hydride (8.9 mL, 0.5 M in toluene) at −78° C. The mixture was stirred overnight at room temperature. The reaction was quenched with water. The mixture was filtered. The filtrate was concentrated to give the title compound (0.2 g).

¹H NMR (CD₃OD): δ 3.51 (s, 2H), 4.58 (s, 2H), 7.14 (d, J=8.0 Hz, 1H), 7.73 (d, J=8.0 Hz, 1H).

### Step 5

### 3-Oxo-3,4-dihydro-2H-pyrido[3,2-b][1,4]thiazine-6-carbaldehyde

A mixture of 6-(hydroxymethyl)-2H-pyrido[3,2-b][1,4]thiazin-3(4H)-one (100 mg) and manganese(IV) oxide (1.2 g) in dichloromethane-1,4-dioxanetetrahydrofuran (5 mL5 mL2 mL) was stirred overnight at room temperature. The mixture was filtered. The filtrate was concentrated to give the title compound (60 mg).

¹H NMR (CDCl₃): δ 3.52 (s, 2H), 7.55 (d, J=8.0 Hz, 1H), 7.75 (d, J=8.0 Hz, 1H), 8.61 (br, 1H), 9.85 (s, 1H).

### Step 6

### 6-((1-(2-(3-Fluoro-6-methoxy-1,5-naphthyridin-4-yl)ethyl)-2-oxabicyclo[2.2.2]octan-4-ylamino)methyl)-2H-pyrido[3,2-b][1,4]thiazin-3(4H)-one

A mixture of 3-oxo-3,4-dihydro-2H-pyrido[3,2-b][1,4]thiazine-6-carbaldehyde (30 mg) and 1-[2-(3-fluoro-6-methoxy-1,5-naphthyridin-4-yl)ethyl]-2-oxabicyclo[2.2.2]octan-4-amine (46 mg) in N,N-dimethylformamide (2 mL) was stirred for 30 minutes at room temperature. Then sodium triacetoxyborohydride (45 mg) was added. The mixture was stirred overnight at room temperature. The mixture was poured into water and extracted with ethyl acetate. The organic phases were washed with brine, dried over sodium sulfate and filtered. The filtrate was concentrated. The residue was purified by prep-HPLC to give the title compound (30 mg).

¹H NMR (CD₃OD): δ 1.83-1.87 (m, 2H), 2.00-2.06 (m, 2H), 2.15-2.20 (m, 6H), 3.26-3.30 (m, 2H), 3.58 (s, 2H), 4.02 (s, 2H), 4.12 (s, 3H), 4.30 (s, 2H), 7.14-7.20 (m, 2H), 7.85 (d, J=8.0 Hz, 1H), 8.22 (d, J=8.0 Hz, 1H), 8.63 (s, 1H).

### Example 156

### 6-((1-(2-(3-Fluoro-6-methoxy-1,5-naphthyridin-4-yl)-1-hydroxyethyl)-2-oxabicyclo[2.2.2]octan-4-ylamino)methyl)-2H-pyrido[3,2-b][1,4]thiazin-3(4H)-one

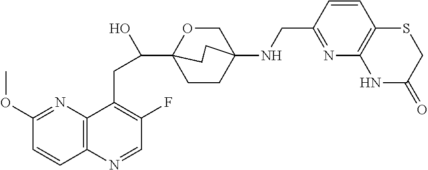 [SMILES: COc1ccc2ncc(F)c(CC(O)C34CCC(NCc5ccc6c(n5)NC(=O)CS6)(CC3)CO4)c2n1]

### Step 1

### 6-((1-(2-(3-Fluoro-6-methoxy-1,5-naphthyridin-4-yl)-1-hydroxyethyl)-2-oxabicyclo[2.2.2]octan-4-ylamino)methyl)-2H-pyrido[3,2-b][1,4]thiazin-3(4H)-one

A mixture of 1-(4-amino-2-oxabicyclo[2.2.2]oct-1-yl)-2-(3-fluoro-6-methoxy-1,5-naphthyridin-4-yl)ethanol (60 mg) and 3-oxo-3,4-dihydro-2H-pyrido[3,2-b][1,4]thiazine-6-carbaldehyde (33 mg) in N,N-dimethylformamide (3 mL) was stirred for 30 minutes at room temperature. Then sodium triacetoxyborohydride (180 mg) was added. The mixture was stirred overnight at room temperature. The mixture was poured into water and extracted with ethyl acetate. The organic phases were washed with brine, dried over sodium sulfate, filtered and concentrated. The residue was purified by prep-HPLC to give the title compound (10 mg).

¹H NMR (CD₃OD): δ 1.97-2.32 (m, 8H), 3.20-3.22 (m, 1H), 3.50-3.54 (m, 3H), 3.96-3.99 (m, 3H), 4.08 (s, 3H), 4.27 (s, 2H), 7.10-7.16 (m, 2H), 7.81 (d, J=8.0 Hz, 1H), 8.19 (d, J=8.8 Hz, 1H), 8.61 (s, 1H).

EXAMPLES 157-187 were prepared according to the methods described above.

### Example 157

### 6-[({1-[1-Fluoro-2-(3-fluoro-6-methoxy-1,5-naphthyridin-4-yl)ethyl]-2-oxabicyclo[2.2.2]oct-4-yl}amino)methyl]-2H-pyrido[3,2-b][1,4]oxazin-3(4H)-one

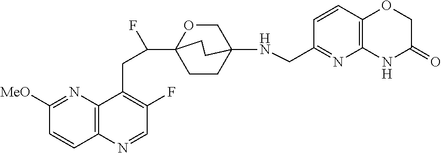 [SMILES: COc1ccc2ncc(F)c(CC(F)C34CCC(NCc5ccc6c(n5)NC(=O)CO6)(CC3)CO4)c2n1]

### Example 158

### 6-{[(1-{2-[3-Fluoro-6-(3-hydroxypropoxy)-1,5-naphthyridin-4-yl]-1-hydroxyethyl}-2-oxabicyclo[2.2.2]oct-4-yl)amino]methyl}-2H-pyrido[3,2-b][1,4]oxazin-3(4H)-one

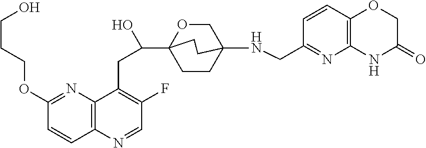 [SMILES: O=C1COc2ccc(CNC34CCC(C(O)Cc5c(F)cnc6ccc(OCCCO)nc56)(CC3)OC4)nc2N1]

### Example 159

### 5-(2-(4-((2,3-Dihydro-[1,4]dioxino[2,3-c]pyridin-7-yl)methylamino)-2-oxabicyclo[2.2.2]octan-1-yl)-2-hydroxyethyl)-6-oxo-5,6-dihydro-1,5-naphthyridine-3-carbonitrile (Enantiomer A)

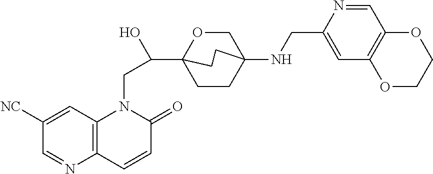 [SMILES: N#Cc1cnc2ccc(=O)n(CC(O)C34CCC(NCc5cc6c(cn5)OCCO6)(CC3)CO4)c2c1]

The title compound (49.0 mg) was prepared from 5-(2-(4-amino-2-oxabicyclo[2.2.2]octan-1-yl)-2-hydroxyethyl)-6-oxo-5,6-dihydro-1,5-naphthyridine-3-carbonitrile (57.4 mg, Enantiomer A) and 2,3-dihydro-[1,4]dioxino[2,3-c]pyridine-7-carbaldehyde (23.8 mg) in the same manner as described for Step 3 of EXAMPLE 1.

¹H NMR (DMSO-d₆) δ 1.58-1.86 (m, 6H), 1.92-2.06 (m, 2H), 3.55 (ddd, J=10.4, 7.3, 2.4 Hz, 1H), 3.60 (s, 2H), 3.62 (s, 2H), 4.17 (dd, J=14.1, 10.4 Hz, 1H), 4.24-4.28 (m, 2H), 4.30-4.34 (m, 2H), 4.41 (dd, J=14.1, 2.4 Hz, 1H), 4.93 (d, J=6.7 Hz, 1H), 6.92 (s, 1H), 7.02 (d, J=9.8 Hz, 1H), 7.98 (s, 1H), 7.99 (d, J=9.8 Hz, 1H), 8.50 (d, J=1.2 Hz, 1H), 8.84 (d, J=1.2 Hz, 1H).

MS (ESI⁺) m/z: 490 (MH⁺).

HRMS (ESI⁺) for C₂₆H₂₈N₅O₅ (MH⁺): calcd, 490.20904. found, 490.20854.

### Preparation of Intermediates

### Step 1

### Preparation of tert-Butyl 1-(2-(7-Bromo-2-oxo-1,5-naphthyridin-1(2H)-yl)-1-hydroxyethyl)-2-oxabicyclo[2.2.2]octan-4-ylcarbamate

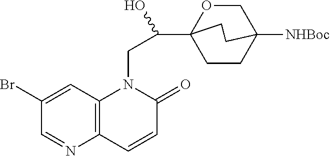 [SMILES: CC(C)(C)OC(=O)NC12CCC(C(O)Cn3c(=O)ccc4ncc(Br)cc43)(CC1)OC2]

To a solution of 7-bromo-1,5-naphthyridin-2(1H)-one (1.10 g) and tert-butyl 1-(oxiran-2-yl)-2-oxabicyclo[2.2.2]octan-4-ylcarbamate (1.38 g) in N,N-dimethylacetamide (16 mL) was added cesium carbonate (3.51 g), the mixture was stirred at 70° C. for 28 hours. After dilution of the mixture with ethyl acetate, the mixture was washed with water. The organic extracts were washed with brine, dried over anhydrous sodium sulfate, filtered, and then concentrated in vacuo. Flash chromatography (silica, hexane:ethyl acetate=1:1) of the residue gave tert-butyl 1-(2-(7-bromo-2-oxo-1,5-naphthyridin-1(2H)-yl)-1-hydroxyethyl)-2-oxabicyclo[2.2.2]octan-4-ylcarbamate (670 mg).

¹H NMR (DMSO-d₆) δ 1.36 (s, 9H), 1.69-1.74 (m, 5H), 1.88-2.02 (m, 3H), 3.50-3.57 (m, 1H), 3.82 (s, 2H), 4.06-4.18 (m, 1H), 4.99 (d, J=5.5 Hz, 1H), 6.63 (brs, 2H), 6.88 (d, J=9.8 Hz, 1H), 7.91 (d, J=9.8 Hz, 1H), 8.24 (d, J=1.2 Hz, 1H), 8.57 (d, J=1.8 Hz, 1H),

MS (ESI⁺) m/z: 494 (MH⁺).

HRMS (ESI⁺) for C₂₂H₂₉BrN₃O₅ (MH⁺): calcd, 494.12906. found, 494.12925.

### Step 2

### Preparation of tert-Butyl 1-(2-(7-Cyano-2-oxo-1,5-naphthyridin-1(2H)-yl)-1-hydroxyethyl)-2-oxabicyclo[2.2.2]octan-4-ylcarbamate

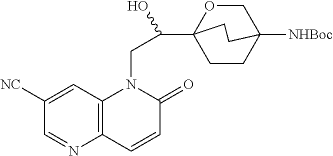 [SMILES: CC(C)(C)OC(=O)NC12CCC(C(O)Cn3c(=O)ccc4ncc(C#N)cc43)(CC1)OC2]

The title compound (604 mg) was prepared from tert-butyl 1-(2-(7-bromo-2-oxo-1,5-naphthyridin-1(2H)-yl)-1-hydroxyethyl)-2-oxabicyclo[2.2.2]octan-4-ylcarbamate (509 mg) in the same manner as described for Step 2 of EXAMPLE 128. Optical resolution (CHIRALPAK IA, TBME:ethanol=7:3) of the racemate gave Enantiomer A (157 mg) and Enantiomer B (159 mg).

Enantiomer A: ¹H NMR (CDCl₃) δ 1.44 (s, 9H), 1.84-2.08 (m, 6H), 2.10-2.25 (m, 2H), 3.19 (d, J=3.7 Hz, 1H), 3.68-3.74 (m, 1H), 4.02-4.14 (m, 2H), 4.22 (dd, J=14.7, 8.6 Hz, 1H), 4.35 (s, 1H), 4.47 (dd, J=14.7, 1.2 Hz, 1H), 7.06 (d, J=9.8 Hz, 1H), 7.98 (d, J=9.8 Hz, 1H), 8.30 (d,. J=1.2 Hz, 1H), 8.73 (d, J=1.8 Hz, 1H),

MS (ESI⁺) m/z: 441 (MH⁺).

HRMS (ESI⁺) for C₂₃H₂₉N₄O₅ (MH⁺): calcd, 441.21379. found, 441.21394.

Enantiomer B: ¹H NMR (CDCl₃) δ 1.44 (s, 9H), 1.82-1.92 (m, 2H), 1.93-2.08 (m, 4H), 2.10-2.26 (m, 2H), 3.19 (d, J=3.7 Hz, 1H), 3.68-3.74 (m, 1H), 4.02-4.14 (m, 2H), 4.22 (dd, J=14.7, 8.6 Hz, 1H), 4.35 (s, 1H), 4.47 (dd, J=14.7, 1.2 Hz, 1H), 7.06 (d, J=9.8 Hz, 1H), 7.98 (d, J=9.8 Hz, 1H), 8.30 (d, J=1.2 Hz, 1H), 8.73 (d, J=1.8 Hz, 1H).

MS (ESI⁺) m/z: 441 (MH⁺).

HRMS (ESI⁺) for C₂₃H₂₉N₄O₅ (MH⁺): calcd, 441.21379. found, 441.21435.

### Step 3

### Preparation of 5-(2-(4-Amino-2-oxabicyclo[2.2.2]octan-1-yl)-2-hydroxyethyl)-6-oxo-5,6-dihydro-1,5-naphthyridine-3-carbonitrile

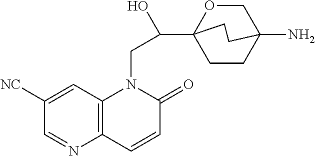 [SMILES: N#Cc1cnc2ccc(=O)n(CC(O)C34CCC(N)(CC3)CO4)c2c1]

The title compound (62.1 mg, Enantiomer A, 61.7 mg, Enantiomer B) was prepared from (79.7 mg, Enantiomer A, 78.0 mg, Enantiomer B) in the same manner as described for Step 2 of EXAMPLE 32.

Enantiomer A: ¹H NMR (DMSO-d₆) δ 1.36 (br, 2H), 1.48-1.64 (m, 4H), 1.66-1.86 (m, 3H), 1.93-2.03 (m, 1H), 3.49 (s, 2H), 3.54 (ddd, J=9.8, 6.7, 3.0 Hz, 1H), 4.16 (d, J=14.1, 9.8 Hz, 1H), 4.41 (dd, J=14.1, 2.4 Hz, 1H), 4.92 (d, J=6.7 Hz, 1H), 7.02 (d, J=9.8 Hz, 1H), 7.99 (d, J=9.8 Hz, 1H), 8.49 (d, J=1.2 Hz, 1H), 8.84 (d, J=1.2 Hz, 1H).

MS (ESI⁺) m/z: 341 (MH⁺).

HRMS (ESI⁺) for C₁₈H₂₁N₄O₃ (MH⁺): calcd, 341.16136. found, 341.16167.

Enantiomer B: ¹H NMR (DMSO-d₆) δ 1.36 (br, 2H), 1.46-1.64 (m, 4H), 1.67-1.86 (m, 3H), 1.92-2.04 (m, 1H), 3.49 (s, 2H), 3.54 (ddd, J=9.8, 6.8, 2.4 Hz, 1H), 4.16 (d, J=14.1, 10.4 Hz, 1H), 4.40 (dd, J=14.1, 2.4 Hz, 1H), 4.92 (d, J=6.8 Hz, 1H), 7.02 (d, J=9.8 Hz, 1H), 7.99 (d, J=9.8 Hz, 1H), 8.49 (s, 1H), 8.84 (d, J=1.2 Hz, 1H).

MS (ESI⁺) m/z: 341 (MH⁺).

HRMS (ESI⁺) for C₁₈H₂₁N₄O₃ (MH⁺): calcd, 341.16136. found, 341.16210.

### Example 160

### 5-(2-Hydroxy-2-(4-((3-oxo-3,4-dihydro-2H-pyrido[3,2-b][1,4]oxazin-6-yl)methylamino)-2-oxabicyclo[2.2.2]octan-1-yl)ethyl)-6-oxo-5,6-dihydro-1,5-naphthyridine-3-carbonitrile (Enantiomer A)

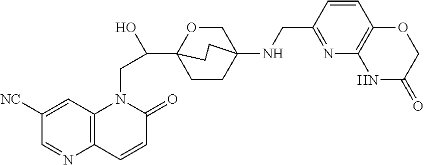 [SMILES: N#Cc1cnc2ccc(=O)n(CC(O)C34CCC(NCc5ccc6c(n5)NC(=O)CO6)(CC3)CO4)c2c1]

¹H NMR (DMSO-d₆) δ 1.58-2.08 (m, 8H), 3.58 (ddd, J=9.8, 6.7, 2.4 Hz, 1H), 3.62 (s, 2H), 3.64 (s, 2H), 4.17 (dd, J=14.1, 2.4 Hz, 1H), 4.42 (dd, J=14.1, 2.4 Hz, 1H), 4.59 (s, 2H), 4.94 (d, J=6.7 Hz, 1H), 7.02 (d, J=8.0 Hz, 1H), 7.02 (d, J=9.8 Hz, 1H), 7.28 (d, J=8.0 Hz, 1H), 7.99 (d, J=9.8 Hz, 1H), 8.50 (s, 1H), 8.84 (d, J=1.8 Hz, 1H), 11.15 (s, 1H).

MS (ESI⁺) m/z: 503 (MH⁺).

HRMS (ESI⁺) for C₂₆H₂₇N₆O₅ (MH⁺): calcd, 503.20429. found, 503.20498.

### Example 161

### 5-(2-(4-((2,3-Dihydro-[1,4]dioxino[2,3-c]pyridin-7-yl)methylamino)-2-oxabicyclo[2.2.2]octan-1-yl)-2-hydroxyethyl)-6-oxo-5,6-dihydro-1,5-naphthyridine-3-carbonitrile (Enantiomer B)

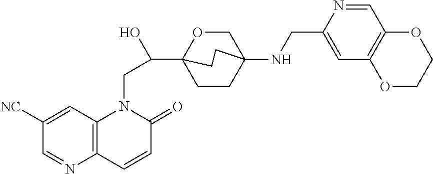 [SMILES: N#Cc1cnc2ccc(=O)n(CC(O)C34CCC(NCc5cc6c(cn5)OCCO6)(CC3)CO4)c2c1]

The title compound (41.8 mg) was prepared from 5-(2-(4-amino-2-oxabicyclo[2.2.2]octan-1-yl)-2-hydroxyethyl)-6-oxo-5,6-dihydro-1,5-naphthyridine-3-carbonitrile (57.4 mg, Enantiomer A) and 2,3-dihydro-[1,4]dioxino[2,3-c]pyridine-7-carbaldehyde (28.0 mg) in the same manner as described for Step 3 of EXAMPLE 1.

¹H NMR (DMSO-d₆) δ 1.58-1.86 (m, 6H), 1.92-2.06 (m, 2H), 3.52-3.58 (m, 1H), 3.60 (s, 2H), 3.62 (br, 2H), 4.17 (dd, J=14.7, 9.8 Hz, 1H), 4.24-4.28 (m, 2H), 4.30-4.34 (m, 2H), 4.41 (dd, J=14.1, 2.4 Hz, 1H), 4.93 (d, J=6.1 Hz, 1H), 6.93 (s, 1H), 7.02 (d, J=9.8 Hz, 1H), 7.98 (s, 1H), 7.99 (d, J=9.8 Hz, 1H), 8.50 (s, 1H), 8.84 (d, J=1.8 Hz, 1H).

MS (ESI⁺) m/z: 490 (MH⁺).

HRMS (ESI⁺) for C₂₆H₂₈N₅O₅ (MH⁺): calcd, 490.20904. found, 490.20891.

### Example 162

### 5-(2-Hydroxy-2-(4-((3-oxo-3,4-dihydro-2H-pyrido[3,2-b][1,4]oxazin-6-yl)methylamino)-2-oxabicyclo[2.2.2]octan-1-yl)ethyl)-6-oxo-5,6-dihydro-1,5-naphthyridine-3-carbonitrile

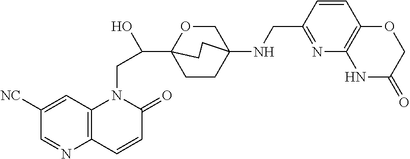 [SMILES: N#Cc1cnc2ccc(=O)n(CC(O)C34CCC(NCc5ccc6c(n5)NC(=O)CO6)(CC3)CO4)c2c1]

¹H NMR (DMSO-d₆) δ 1.58-2.08 (m, 8H), 3.58 (ddd, J=9.8, 6.7, 2.4 Hz, 1H), 3.62 (s, 2H), 3.64 (s, 2H), 4.17 (dd, J=14.1, 2.4 Hz, 1H), 4.42 (dd, J=14.1, 2.4 Hz, 1H), 4.59 (s, 2H), 4.94 (d, J=6.7 Hz, 1H), 7.02 (d, J=8.0 Hz, 1H), 7.02 (d, J=9.8 Hz, 1H), 7.28 (d, J=8.6 Hz, 1H), 7.99 (d, J=9.8 Hz, 1H), 8.50 (d, J=1.2 Hz, 1H), 8.84 (d, J=1.8 Hz, 1H), 11.15 (s, 1H).

MS (ESI⁺) m/z: 503 (MH⁺).

HRMS (ESI⁺) for C₂₆H₂₇N₆O₅ (MH⁺): calcd, 503.20429. found, 503.20426.

### Example 163

### 7-((1-(2-(3-Fluoro-6-methoxy-1,5-naphthyridin-4-yl)ethyl)-2-oxabicyclo[2.2.2]octan-4-ylamino)methyl)-1H-pyrido[2,3-e][1,3,4]oxathiazine-2,2-dioxide

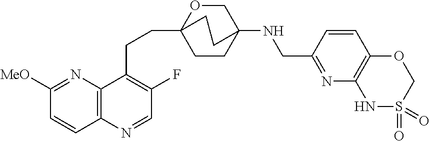 [SMILES: COc1ccc2ncc(F)c(CCC34CCC(NCc5ccc6c(n5)NS(=O)(=O)CO6)(CC3)CO4)c2n1]

¹H NMR (DMSO-d₆) δ 1.58-1.92 (m, 10H), 2.14 (brs, 1H), 3.08-3.14 (m, 2H), 3.56-3.64 (m, 4H), 4.03 (s, 3H), 5.37 (s, 2H), 6.47 (d, J=8.0 Hz, 1H), 6.95 (d, J=8.0 Hz, 1H), 7.22 (d, J=9.2 Hz, 1H), 8.26 (d, J=8.6 Hz, 1H), 8.74 (s, 1H), 10.08 (brs, 1H).

MS (ESI⁺) m/z: 530 (MH⁺).

HRMS (ESI⁺) for C₂₅H₂₉FN₅O₅S (MH⁺): calcd, 530.18734. found, 530.18643.

### Example 164

### 6-((1-(2-(6-(((2S,3R)-3-Amino-4-oxoazetidin-2-yl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

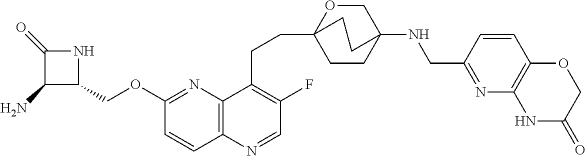 [SMILES: N[C@H]1C(=O)N[C@@H]1COc1ccc2ncc(F)c(CCC34CCC(NCc5ccc6c(n5)NC(=O)CO6)(CC3)CO4)c2n1]

¹H NMR (DMSO-d₆) δ 1.58-1.81 (m, 8H), 1.81-1.95 (m, 2H), 3.04-3.16 (m, 2H), 3.62 (s, 2H), 3.64-3.73 (m, 3H), 3.88 (d, J=1.8 Hz, 1H), 4.51 (dd, J=11.6, 6.7 Hz, 1H), 4.60 (s, 2H), 4.70 (dd, J=11.6, 3.7 Hz, 1H), 7.03 (d, J=7.9 Hz, 1H), 7.23 (d, J=8.6 Hz, 1H), 7.30 (d, J=7.9 Hz, 1H), 8.22 (s, 1H), 8.29 (d, J=9.2 Hz, 1H), 8.76 (s, 1H), 11.17 (s, 1H).

MS (ESI⁺) m/z: 578 (MH⁺).

HRMS (ESI⁺) for C₂₉H₃₃FN₇O₅ (MH⁺): calcd, 578.25272. found, 578.25268.

### Example 165

### 6-{[(1-{2-[6-(3-Amino-2-hydroxypropoxy)-3-fluoro-1,5-naphthyridin-4-yl]ethyl}-2-oxabicyclo[2.2.2]oct-4-yl)amino]methyl}-2H-pyrido[3,2-b][1,4]oxazin-3(4H)-one

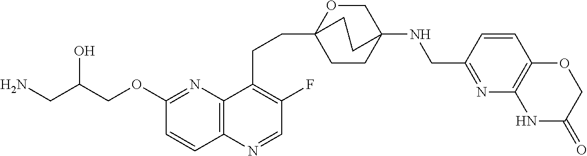 [SMILES: NCC(O)COc1ccc2ncc(F)c(CCC34CCC(NCc5ccc6c(n5)NC(=O)CO6)(CC3)CO4)c2n1]

### Example 166

### 6-({[1-(2-{6-[(3,5-Dihydroxycyclohexyl)oxy]-3-fluoro-1,5-naphthyridin-4-yl}ethyl)-2-oxabicyclo[2.2.2]oct-4-yl]amino}methyl)-2H-pyrido[3,2-b][1,4]oxazin-3(4H)-one

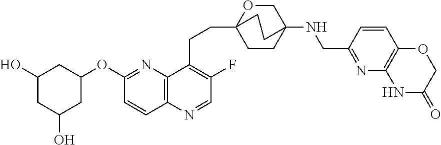 [SMILES: O=C1COc2ccc(CNC34CCC(CCc5c(F)cnc6ccc(OC7CC(O)CC(O)C7)nc56)(CC3)OC4)nc2N1]

### Example 167

### 6-[({1-[2-(3-Fluoro-6-methoxy-1,5-naphthyridin-4-yl)-1-(2-hydroxyethoxy)ethyl]-2-oxabicyclo[2.2.2]oct-4-yl}amino)methyl]-2H-pyrido[3,2-b][1,4]oxazin-3(4H)-one

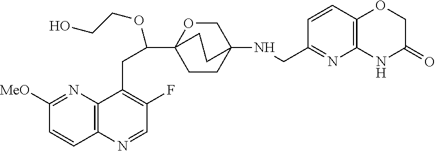 [SMILES: COc1ccc2ncc(F)c(CC(OCCO)C34CCC(NCc5ccc6c(n5)NC(=O)CO6)(CC3)CO4)c2n1]

### Example 168

### Methyl {1-[2-(3-Fluoro-6-methoxy-1,5-naphthyridin-4-yl)ethyl]-2-oxabicyclo[2.2.2]oct-4-yl}[(3-oxo-3,4-dihydro-2H-pyrido[3,2-b][1,4]oxazin-6-yl)methyl]carbamate

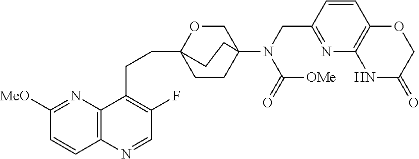 [SMILES: COC(=O)N(Cc1ccc2c(n1)NC(=O)CO2)C12CCC(CCc3c(F)cnc4ccc(OC)nc34)(CC1)OC2]

### Example 169

### 6-((1-(2-(6-(((1r,3R,4S)-3,4-Dihydroxycyclopentyl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

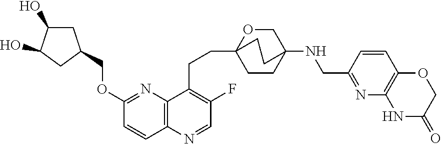 [SMILES: O=C1COc2ccc(CNC34CCC(CCc5c(F)cnc6ccc(OC[C@H]7C[C@@H](O)[C@@H](O)C7)nc56)(CC3)OC4)nc2N1]

¹H NMR (CDCl₃) δ 1.64-1.84 (m, 10H), 2.03-2.06 (m, 2H), 2.17-2.24 (m, 2H), 2.48-2.54 (m, 1H), 3.21 (d, J=7.9 Hz, 2H), 3.77 (s, 2H), 3.79 (s, 2H), 4.12-4.14 (m, 2H), 4.53 (d, J=7.3 Hz, 2H), 4.64 (s, 2H), 6.95 (d, J=7.9 Hz, 1H), 7.06 (d, J=9.2 Hz, 1H), 7.21 (d, J=8.6 Hz, 1H), 8.18 (d, J=9.2 Hz, 1H), 8.60 (s, 1H).

MS (ESI⁺) m/z: 594 (MH⁺).

HRMS (ESI) for C₃₁H₃₆FN₅O₆ (MH⁺): calcd, 594.27279. found, 594.27289.

### Example 170

### 6-((1-(2-(3-Fluoro-6-((3-hydroxyoxetan-3-yl)methoxy)-1,5-naphthyridin-4-yl)ethyl)-2-oxabicyclo[2.2.2]octan-4-ylamino)methyl)-2H-pyrido[3,2-b][1,4]oxazin-3(4H)-one

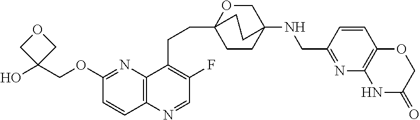 [SMILES: O=C1COc2ccc(CNC34CCC(CCc5c(F)cnc6ccc(OCC7(O)COC7)nc56)(CC3)OC4)nc2N1]

¹H NMR (DMSO-d₆) □ 1.78-1.58 (m, 8H), 1.93-1.82 (m, 3H), 3.14-3.07 (m, 2H), 3.58 (s, 2H), 3.65-3.60 (m, 2H), 4.51 (d, J=6.7 Hz, 2H), 4.53 (d, J=6.7 Hz, 2H), 4.59 (s, 2H), 4.64 (s, 2H), 6.09 (s, 1H), 7.01 (d, J=8.0 Hz, 1H), 7.25 (d, J=9.2 Hz, 1H), 7.27 (d, J=8.0 Hz, 1H), 8.28 (d, J=9.2 Hz, 1H), 8.75 (s, 1H), 11.14 (brs, 1H).

MS (ESI⁺) m/z: 566 (MH⁺).

HRMS (ESI⁺) for C₂₉H₃₃FN₅O₆ (MH⁺): calcd, 566.24149. found, 566.24171.

### Example 171

### 6-((1-(2-(3-Fluoro-6-(2-hydroxyethoxy)-1,5-naphthyridin-4-yl)-1-hydroxyethyl)-2-oxabicyclo[2.2.2]octan-4-ylamino)methyl)-2H-pyrido[3,2-b][1,4]oxazin-3(4H)-one Hydrochloride

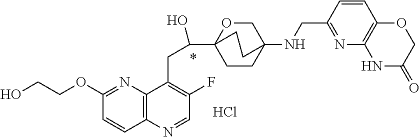 [SMILES: O=C1COc2ccc(CNC34CCC(C(O)Cc5c(F)cnc6ccc(OCCO)nc56)(CC3)OC4)nc2N1 ; Cl]

¹H NMR (DMSO-d₆) δ 1.81-2.14 (m, 8H), 3.01 (dd, J=11.6, 11.0 Hz, 1H), 3.34 (d, J=12.2 Hz, 1H), 3.72-3.84 (m, 5H), 3.90 (s, 2H), 4.11 (t, J=5.5 Hz, 2H), 4.48 (t, J=4.9 Hz, 2H), 4.69 (s, 2H), 7.21 (d, J=9.2 Hz, 2H), 7.46 (d, J=7.9 Hz, 1H), 8.27 (d, J=9.2 Hz, 1H), 8.74 (s, 1H), 9.28 (s, 2H), 11.33 (s, 1H).

MS (ESI⁺) m/z: 540 (MH⁺) (as free base).

HRMS (ESI⁺) for C₂₇H₃₁FN₅O₆ (MH⁺) (as free base): calcd, 540.22584. found, 540.22538.

### Example 172

### 1-{4-[(2,3-Dihydro-1,4-benzodioxin-6-ylmethyl)amino]-2-oxabicyclo[2.2.2]oct-1-yl}-2-(3-fluoro-6-methoxy-1,5-naphthyridin-4-yl)ethanol

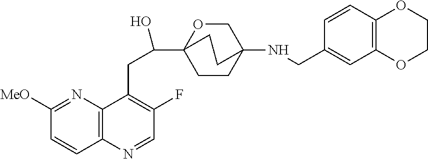 [SMILES: COc1ccc2ncc(F)c(CC(O)C34CCC(NCc5ccc6c(c5)OCCO6)(CC3)CO4)c2n1]

### Example 173

### 3-Fluoro-N-{1-[2-(3-fluoro-6-methoxy-1,5-naphthyridin-4-yl)-1-hydroxyethyl]-2-oxabicyclo[2.2.2]oct-4-yl}-4-methylbenzamide

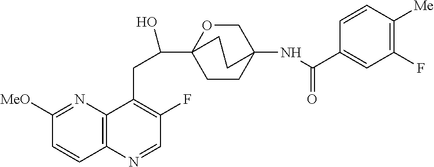 [SMILES: COc1ccc2ncc(F)c(CC(O)C34CCC(NC(=O)c5ccc(C)c(F)c5)(CC3)CO4)c2n1]

### Example 174

### 6-[({1-[2-(3,7-Difluoro-6-methoxy-1,5-naphthyridin-4-yl)ethyl]-2-oxabicyclo[2.2.2]oct-4-yl}amino)methyl]-2H-pyrido[3,2-b][1,4]oxazin-3(4H)-one

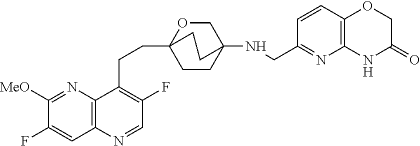 [SMILES: COc1nc2c(CCC34CCC(NCc5ccc6c(n5)NC(=O)CO6)(CC3)CO4)c(F)cnc2cc1F]

### Example 175

### 6-{[(1-{2-[3-Fluoro-6-(3-hydroxy-3-methylbutoxy)-1,5-naphthyridin-4-yl]ethyl}-2-oxabicyclo[2.2.2]oct-4-yl)amino]methyl}-2H-pyrido[3,2-b][1,4]oxazin-3(4H)-one

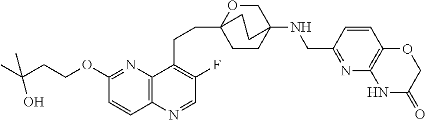 [SMILES: CC(C)(O)CCOc1ccc2ncc(F)c(CCC34CCC(NCc5ccc6c(n5)NC(=O)CO6)(CC3)CO4)c2n1]

### Example 176

### 6-{[{1-[2-(3-Fluoro-6-methoxy-1,5-naphthyridin-4-yl)ethyl]-2-oxabicyclo[2.2.2]oct-4-yl}(methyl)amino]methyl}-2H-pyrido[3,2-b][1,4]oxazin-3(4H)-one

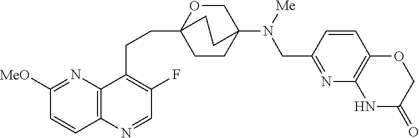 [SMILES: COc1ccc2ncc(F)c(CCC34CCC(N(C)Cc5ccc6c(n5)NC(=O)CO6)(CC3)CO4)c2n1]

### Example 177

### 3-[({1-[2-(3-Fluoro-6-methoxy-1,5-naphthyridin-4-yl)ethyl]-2-oxabicyclo[2.2.2]oct-4-yl}amino)methyl]-5H-pyridazino [3,4-b][1,4]oxazin-6(7H)-one

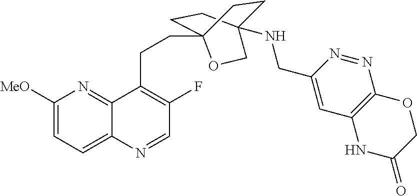 [SMILES: COc1ccc2ncc(F)c(CCC34CCC(NCc5cc6c(nn5)OCC(=O)N6)(CC3)CO4)c2n1]

### Example 178

### 6-[({1-[2-(3-Fluoro-6-methoxy-1,5-naphthyridin-4-yl)ethyl]-2-oxabicyclo[2.2.2]oct-4-yl}amino)methyl]pyrido[2,3-b]pyrazin-3(4H)-one

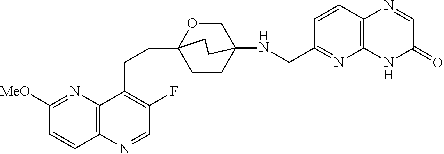 [SMILES: COc1ccc2ncc(F)c(CCC34CCC(NCc5ccc6ncc(=O)[nH]c6n5)(CC3)CO4)c2n1]

### Example 179

### 7-[({1-[2-(3-Fluoro-6-methoxy-1,5-naphthyridin-4-yl)ethyl]-2-oxabicyclo[2.2.2]oct-4-yl}amino)methyl]pyrido[3,4-b]pyrazin-2(1H)-one

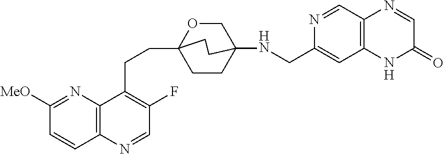 [SMILES: COc1ccc2ncc(F)c(CCC34CCC(NCc5cc6[nH]c(=O)cnc6cn5)(CC3)CO4)c2n1]

### Example 180

### 2-Oxo-1-[2-(4-{[(3-oxo-3,4-dihydro-2H-pyrido[3,2-b][1,4]oxazin-6-yl)methyl]amino}-2-oxabicyclo[2.2.2]oct-1-yl)ethyl]-1,2-dihydroquinoline-7-carbonitrile

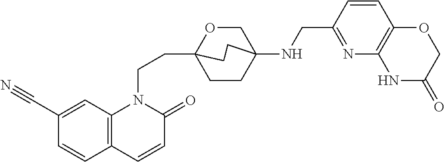 [SMILES: N#Cc1ccc2ccc(=O)n(CCC34CCC(NCc5ccc6c(n5)NC(=O)CO6)(CC3)CO4)c2c1]

### Example 181

### 6-{[(1-{2-[3-Fluoro-6-(3-hydroxybutoxy)-1,5-naphthyridin-4-yl]ethyl}-2-oxabicyclo[2.2.2]oct-4-yl)amino]methyl}-2H-pyrido[3,2-b][1,4]oxazin-3(4H)-one

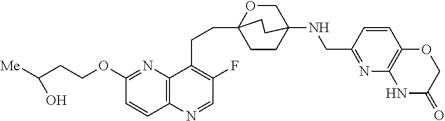 [SMILES: CC(O)CCOc1ccc2ncc(F)c(CCC34CCC(NCc5ccc6c(n5)NC(=O)CO6)(CC3)CO4)c2n1]

### Example 182

### 7-[({1-[2-(3-Fluoro-6-methoxy-1,5-naphthyridin-4-yl)-1-hydroxyethyl]-2-oxabicyclo[2.2.2]oct-4-yl}amino)methyl]pyrido[3,4-b]pyrazin-2(1H)-one

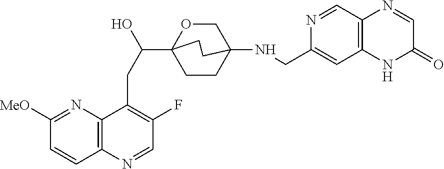 [SMILES: COc1ccc2ncc(F)c(CC(O)C34CCC(NCc5cc6[nH]c(=O)cnc6cn5)(CC3)CO4)c2n1]

### Example 183

### 3-((1-(2-(3-Fluoro-6-methoxy-1,5-naphthyridin-4-yl)ethyl)-2-oxabicyclo[2.2.2]octan-4-ylamino)methyl)-1-methyl-1,8-naphthyridin-2(1H)-one Hydrochloride

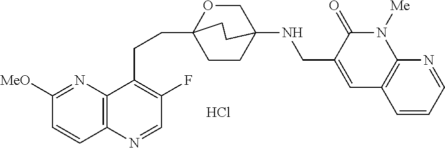 [SMILES: COc1ccc2ncc(F)c(CCC34CCC(NCc5cc6cccnc6n(C)c5=O)(CC3)CO4)c2n1 ; Cl]

¹H NMR (DMSO-d₆) δ 1.70-1.78 (m, 2H), 1.84-1.90 (m, 2H), 1.95-2.19 (m, 6H), 3.06-3.16 (m, 2H), 3.76 (s, 3H), 3.93 (s, 2H), 4.05 (s, 5H), 7.24 (d, J=8.6 Hz, 1H), 7.41 (dd, J=7.3, 4.9 Hz, 1H), 8.25-8.29 (br, 3H), 8.72 (dd, J=4.9, 1.8 Hz, 1H), 8.77 (s, 1H), 9.11 (s, 2H).

MS (ESI⁺) m/z: 504 (MH⁺) (as free base).

HRMS (ESI⁺) for C₂₈H₃₁FN₅O₃ (MH⁺) (as free base): calcd, 504.24109. found, 504.24144.

### Example 184

### 6-((1-(2-(6-(((2S,3R)-3-Aminooxetan-2-yl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

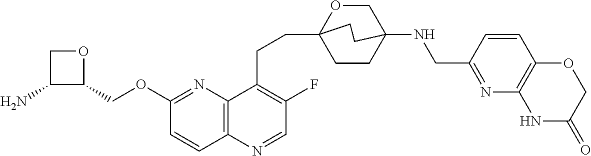 [SMILES: N[C@@H]1CO[C@@H]1COc1ccc2ncc(F)c(CCC34CCC(NCc5ccc6c(n5)NC(=O)CO6)(CC3)CO4)c2n1]

The title compound (23.4 mg) was prepared from benzyl (3R,4S)-4-(7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)tetrahydrofuran-3-ylcarbamate (46.0 mg) in the same manner as described for Step 4 of EXAMPLE 38.

¹H NMR (DMSO-d₆) δ 1.55-1.81 (m, 8H), 1.82-2.05 (m, 2H), 3.04-3.19 (m, 2H), 3.60 (s, 2H), 3.66 (s, 2H), 4.24 (dd, J=14.0, 7.3 Hz, 1H), 4.36 (t, J=6.7 Hz, 1H), 4.60 (s, 2H), 4.70 (dd, J=8.0, 6.7 Hz, 1H), 4.77 (dd, J=12.2, 6.7 Hz, 1H), 4.83 (dd, J=12.2, 4.3 Hz, 1H), 4.98-5.04 (m, 1H), 7.02 (d, J=8.0 Hz, 1H), 7.28 (d, J=9.2 Hz, 1H), 7.29 (d, J=8.0 Hz, 1H), 8.28 (d, J=9.2 Hz, 1H), 8.75 (s, 1H), 11.16 (s, 1H).

MS (ESI⁺) m/z: 565 (MH⁺).

HRMS (ESI⁺) for C₂₉H₃₄FN₆O₅ (MH⁺): calcd, 565.25747. found, 565.25711.

### Preparation of Intermediates

### Step 1

### Preparation of (2R,3S)-2-Azido-4-(benzyloxy)-3-hydroxybutyl 4-methylbenzenesulfonate

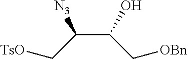 [SMILES: Cc1ccc(S(=O)(=O)OC[C@@H](N=[N+]=[N-])[C@H](O)COCc2ccccc2)cc1]

To a solution of (2R,3S)-2-azido-4-(benzyloxy)butane-1,3-diol (3.30 g), 4-(dimethylamino)pyridine (13.6 mg) and triethylamine (4.56 mL) in dichloromethane (28.4 mL) was added p-toluenesulfonyl chloride (3.19 g) at 0° C., the mixture was stirred at room temperature for 1.5 hours. After dilution of the mixture with dichloromethane, the mixture was washed with water. The organic extracts were washed with brine, dried over anhydrous sodium sulfate, filtered, and then concentrated in vacuo. Flash chromatography (silica, hexane:ethyl acetate=3:1) of the residue gave the title compound (4.07 g).

¹H NMR (CDCl₃) δ 2.21 (d, J=6.1 Hz, 1H), 2.45 (s, 3H), 3.48-3.57 (m, 2H), 3.72-3.78 (m, 1H), 3.85-3.92 (m, 1H), 4.15 (dd, J=10.4, 8.0 Hz, 1H), 4.25 (dd, J=10.4, 4.3 Hz, 1H), 4.53 (s, 2H), 7.28-7.39 (m, 7H), 7.80 (dd, J=6.1, 1.8 Hz, 1H).

MS (ESI⁺) m/z: 409 (M+NH₄).

HRMS (FAB⁺) for C₁₈H₂₅N₄O₅S₁(M+NH₄): calcd, 409.15456. found, 409.15424.

### Step 2

### Preparation of (2S,3R)-3-Azido-2-(benzyloxymethyl)oxetane

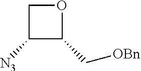 [SMILES: [N-]=[N+]=N[C@@H]1CO[C@@H]1COCc1ccccc1]

To a solution of (2R,3S)-2-azido-4-(benzyloxy)-3-hydroxybutyl 4-methylbenzenesulfonate (3.60 g) in tetrahydrofuran (74 mL) was added potassium t-butoxide (1.44 g) at 0° C., the mixture was stirred at room temperature for 1.5 hours. After dilution of the mixture with ethyl acetate, the mixture was washed with water. The organic extracts were washed with brine, dried over anhydrous sodium sulfate, filtered, and then concentrated in vacuo. Flash chromatography (silica, toluene:acetonitrile=10:1) of the residue gave the title compound (919 mg).

¹H NMR (CDCl₃) δ 3.66 (ddd, J=14.5, 10.6, 3.0 Hz, 2H), 4.40 (t, J=6.1 Hz), 4.54 (dd, J=12.8, 5.5 Hz, 1H), 4.61 (dd, J=17.1, 11.6 Hz, 2H), 4.69 (t, J=6.7 Hz, 1H), 4.73-4.78 (m, 1H), 7.24-7.40 (m, 5H).

MS (CI⁺) m/z: 220 (MH⁺).

HRMS (CI⁺) for C₁₁H₁₄N₃O₃ (MH⁺): calcd, 220.1086. found, 220.1096.

### Step 3

### Preparation of (2S,3R)-2-(Benzyloxymethyl)oxetan-3-amine

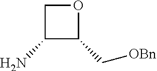 [SMILES: N[C@@H]1CO[C@@H]1COCc1ccccc1]

To a solution of (2S,3R)-3-azido-2-(benzyloxymethyl)oxetane (819 mg) in tetrahydrofuran (9.3 mL) was added triphenylphosphine (1.08 g) at 0° C., the mixture was stirred at room temperature for 4 hours. Water (0.2 mL) was added to the solution, the mixture was stirred at 50° C. for 2 hours, and then concentrated in vacuo. After dilution of the residue with ethyl acetate, the mixture was extracted with 1M hydrochloric acid. The aqueous extracts were made to alkaline by the addition of aqueous 1 M sodium hydroxide solution. The resulting mixture was extracted with ethyl acetate. The organic extracts were dried over anhydrous sodium sulfate, filtered, and then concentrated in vacuo. Flash chromatography (silica, dichloromethane:methanol=10:1) of the residue gave the title compound (681 mg).

¹H NMR (CDCl₃) δ 3.85 (ddd, J=18.4, 11.0, 3.7 Hz, 1H), 4.26 (q, J=7.3 Hz, 1H), 4.44 (dd, J=6.7, 6.1 Hz, 1H), 4.66 (dd, J=44.0, 12.2 Hz, 1H), 4.79 (dd, J=8.0, 6.1 Hz, 1H), 4.81-4.85 (m, 1H), 7.27-7.40 (m, 5H).

MS (CI⁺) m/z: 194 (MH⁺).

HRMS (CI⁺) for C₁₁H₁₆NO₂ (MH⁺): calcd, 194.1181. found, 194.1191.

### Step 4

### Preparation of benzyl (2S,3R)-2-(Hydroxymethyl)oxetan-3-ylcarbamate

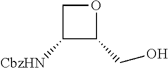 [SMILES: O=C(N[C@@H]1CO[C@@H]1CO)OCc1ccccc1]

A suspension of (2S,3R)-2-(benzyloxymethyl)oxetan-3-amine (200 mg), ammonium formate (326 mg), Pd—C (30.0 mg) in methanol (5.1 mL) and water (5.1 mL) was heated under reflux for 17 hours. After insoluble materials were filtered off, the filtrate was concentrated in vacuo. A mixture of the residue, sodium hydrogencarbonate (261 mg) and water (5.1 mL) was added a solution of benzyl chloroformate (238 mg) in tetrahydrofuran (5.1 mL) at 0° C., the mixture was stirred at room temperature for 3 hours. The mixture was extracted with ethyl acetate. The organic extracts were washed with brine, dried over anhydrous sodium sulfate, filtered, and then concentrated in vacuo. Flash chromatography (silica, hexane:ethyl acetate=1:2) of the residue gave the title compound (182 mg).

¹H NMR (CDCl₃) δ 2.46 (dd, J=9.8, 3.0 Hz, 1H), 3.78 (dd, J=12.8, 9.8 Hz, 1H), 3.96 (ddd, J=12.6, 6.7, 3.1 Hz, 1H), 4.45 (t, J=6.7 Hz, 1H), 4.86-4.98 (m, 2H), 5.04-5.21 (m, 1H), 5.10 (dd, J=18.4, 12.2 Hz, 2H), 5.22 (d, J=9.8 Hz, 1H), 7.29-7.44 (m, 5H).

MS (CI⁺) m/z: 238 (MH⁺).

HRMS (CI⁺) for C₁₂H₁₆NO₄ (MH⁺): calcd, 238.1079. found, 238.1096.

### Step 5

### Preparation of benzyl (2S,3R)-2-(Bromomethyl)oxetan-3-ylcarbamate

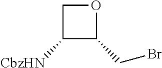 [SMILES: O=C(N[C@@H]1CO[C@@H]1CBr)OCc1ccccc1]

The title compound (103 mg) was prepared from benzyl (2S,3R)-2-(hydroxymethyl)oxetan-3-ylcarbamate (170 mg) in the same manner as described for X.

¹H NMR (CDCl₃) δ 3.48-3.64 (m, 2H), 4.38-4.53 (m, 1H), 4.82-4.94 (m, 1H), 4.99-5.10 (m, 2H), 5.12 (s, 2H), 5.42 (brs, 1H), 7.30-7.43 (m, 5H).

MS (CI⁺) m/z: 300 (MH⁺).

HRMS (CI⁺) for C₁₂H₁₅BrNO₃ (MH⁺): calcd, 300.0235. found, 300.0236.

### Step 6

### Preparation of tert-butyl 1-(2-(6-(((2S,3R)-3-Benzyloxycarbonylaminooxetan-2-yl)methoxy)-3-fluoro-1,5-naphthyridin-4-yl)ethyl)-2-oxabicyclo[2.2.2]octan-4-ylcarbamate

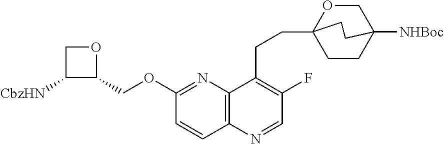 [SMILES: CC(C)(C)OC(=O)NC12CCC(CCc3c(F)cnc4ccc(OC[C@H]5OC[C@H]5NC(=O)OCc5ccccc5)nc34)(CC1)OC2]

The title compound (132 mg) was prepared from tert-butyl 1-(2-(3-fluoro-6-hydroxy-1,5-naphthyridin-4-yl)ethyl)-2-oxabicyclo[2.2.2]octan-4-ylcarbamate (126 mg) and benzyl (2S,3R)-2-(bromomethyl)oxetan-3-ylcarbamate (100 mg) in the same manner as described for Step 1 of EXAMPLE 32.

¹H NMR (CDCl₃) δ 1.43 (s, 9H), 1.62-1.91 (m, 6H), 1.91-2.15 (m, 4H), 3.07-3.25 (m, 2H), 3.89-4.00 (m, 2H), 4.26 (brs, 1H), 4.55-4.66 (m, 2H), 4.87-4.98 (m, 2H), 5.00-5.16 (m, 2H), 5.17-5.31 (m, 2H), 5.93 (d, J=6.7 Hz, 1H), 7.11 (d, J=9.2 Hz, 1H), 7.20 (brs, 1H), 7.23-7.32 (m, 5H), 8.20 (d, J=9.2 Hz, 1H), 8.61 (s, 1H).

MS (ESI⁺) m/z: 637 (MH⁺).

HRMS (ESI⁺) for C₃₄H₄₂FN₄O₇ (MH⁺): calcd, 637.30375. found, 637.30315.

### Step 7

### Preparation of benzyl (2S,3R)-2-((8-(2-(4-Amino-2-oxabicyclo[2.2.2]octan-1-yl)ethyl)-7-fluoro-1,5-naphthyridin-2-yloxy)methyl)oxetan-3-ylcarbamate

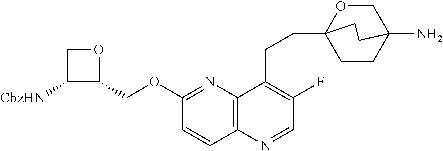 [SMILES: NC12CCC(CCc3c(F)cnc4ccc(OC[C@H]5OC[C@H]5NC(=O)OCc5ccccc5)nc34)(CC1)OC2]

The title compound (86.0 mg) was prepared from tert-butyl 1-(2-(6-(((2S,3R)-3-benzyloxycarbonylaminooxetan-2-yl)methoxy)-3-fluoro-1,5-naphthyridin-4-yl)ethyl)-2-oxabicyclo[2.2.2]octan-4-ylcarbamate (128 mg) in the same manner as described for Step 2 of EXAMPLE 32.

¹H NMR (CDCl₃) δ 1.58-1.78 (m, 8H), 1.90-2.03 (m, 2H), 3.08-3.24 (m, 2H), 3.59-3.66 (m, 2H), 4.56-4.67 (m, 2H), 4.85-4.98 (m, 2H), 4.99-5.16 (m, 2H), 5.17-5.33 (m, 2H), 5.88 (d, J=8.6 Hz, 1H), 7.12 (d, J=8.6 Hz, 1H), 7.15-7.32 (m, 5H), 8.21 (d, J=9.2 Hz, 1H), 8.62 (s, 1H).

MS (ESI⁺) m/z: 537 (MH⁺).

HRMS (ESI⁺) for C₂₉H₃₄FN₄O₅ (MH⁺): calcd, 537.25132. found, 537.25127.

### Step 8

### Preparation of benzyl (2S,3R)-2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)oxetan-3-ylcarbamate

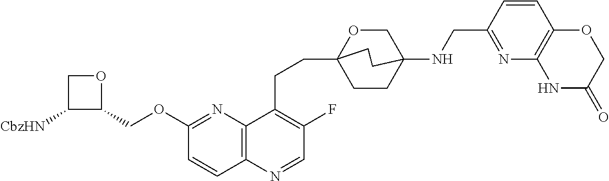 [SMILES: O=C1COc2ccc(CNC34CCC(CCc5c(F)cnc6ccc(OC[C@H]7OC[C@H]7NC(=O)OCc7ccccc7)nc56)(CC3)OC4)nc2N1]

The title compound (77.3 mg) was prepared from benzyl (2S,3R)-2-((8-(2-(4-amino-2-oxabicyclo[2.2.2]octan-1-yl)ethyl)-7-fluoro-1,5-naphthyridin-2-yloxy)methyl)oxetan-3-ylcarbamate (85.0 mg) and 3-oxo-3,4-dihydro-2H-pyrido[3,2-b][1,4]oxazine-6-carbaldehyde (29.6 mg) in the same manner as described for Step 3 of EXAMPLE 1.

¹H NMR (CDCl₃) δ 1.64-1.85 (m, 8H), 1.92-2.04 (m, 2H), 3.08-3.27 (m, 2H), 3.73 (s, 2H), 3.75 (s, 2H), 4.53-4.69 (m, 4H), 4.84-4.99 (m, 2H), 5.00-5.32 (m, 4H), 5.82-5.92 (m, 1H), 6.93 (d, J=8.0 Hz, 1H), 7.12 (d, J=8.6 Hz, 1H), 7.16-7.24 (m, 2H), 7.19 (d, J=8.6 Hz, 1H), 7.24-7.32 (m, 5H), 8.21 (d, J=9.2 Hz, 1H), 8.62 (s, 1H).

MS (ESI⁺) m/z: 699 (MH⁺).

HRMS (ESI⁺) for C₃₇H₄₀FN₆O₇ (MH⁺): calcd, 699.29425. found, 699.29366.

### Example 185

### 6-((1-(2-(6-(((2R,3R)-3-Aminooxetan-2-yl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

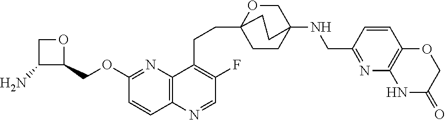 [SMILES: N[C@@H]1CO[C@H]1COc1ccc2ncc(F)c(CCC34CCC(NCc5ccc6c(n5)NC(=O)CO6)(CC3)CO4)c2n1]

¹H NMR (DMSO-d₆) δ 1.54-1.79 (m, 8H), 1.80-1.93 (m, 2H), 3.03-3.14 (m, 2H), 3.58 (s, 2H), 3.63 (s, 2H), 3.82 (dd, J=14.1, 6.7 Hz, 1H), 4.18 (t, J=6.1 Hz, 1H), 4.54 (dd, J=7.4, 6.1 Hz, 1H), 4.58-4.65 (m, 4H), 4.67-4.74 (m, 1H), 7.01 (d, J=7.9 Hz, 1H), 7.26 (d, J=8.6 Hz, 1H), 7.28 (d, J=7.9 Hz, 1H), 8.28 (d, J=8.6 Hz, 1H), 8.75 (s, 1H).

MS (ESI⁺) m/z: 565 (MH⁺).

HRMS (ESI⁺) for C₂₉H₃₄FN₆O₅ (MH⁺): calcd, 565.25747. found, 565.25750.

### Example 186

### 6-{[(1-{2-[3-Fluoro-6-(prop-2-en-1-yloxy)-1,5-naphthyridin-4-yl]ethyl}-2-oxabicyclo[2.2.2]oct-4-yl)amino]methyl}-2H-pyrido[3,2-b][1,4]oxazin-3(4H)-one

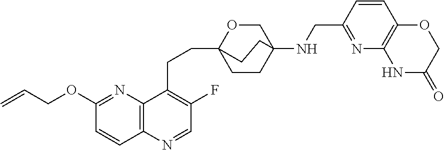 [SMILES: C=CCOc1ccc2ncc(F)c(CCC34CCC(NCc5ccc6c(n5)NC(=O)CO6)(CC3)CO4)c2n1]

### Example 187

### 6-[({5-[2-(3-Fluoro-6-methoxy-1,5-naphthyridin-4-yl)ethyl]-6-oxabicyclo[3.2.2]non-1-yl}amino)methyl]-2H-pyrido[3,2-b][1,4]oxazin-3(4H)-one

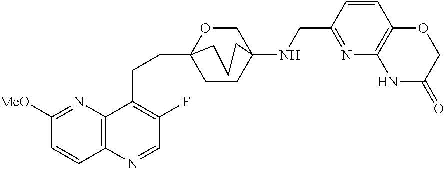 [SMILES: COc1ccc2ncc(F)c(CCC34CCCC(NCc5ccc6c(n5)NC(=O)CO6)(CC3)CO4)c2n1]

### Example 188

### (E)-N-(3-(2,5-Difluorophenyl)allyl)-1-(2-(3-fluoro-6-methoxy-1,5-naphthyridin-4-yl)ethyl)-2-oxabicyclo[2.2.2]octan-4-amine

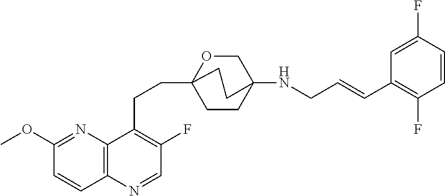 [SMILES: COc1ccc2ncc(F)c(CCC34CCC(NC/C=C/c5cc(F)ccc5F)(CC3)CO4)c2n1]

The title compound was prepared from (E)-3-(2,5-difluorophenyl)acrylaldehyde in the same manner as described for Step 3 of EXAMPLE 1.

¹H NMR (DMSO-d₆): δ 1.58-1.77 (m, 9H), 1.81-1.92 (m, 2H), 3.08-3.16 (m, 2H), 3.59 (s, 2H), 4.03 (s, 3H), 6.47 (dt, J=16.5, 5.5 Hz, 1H), 6.59 (d, J=16.5 Hz, 1H), 7.04-7.11 (m, 2H), 7.42-7.47 (m, 1H), 8.26 (d, J=9.2 Hz), 8.74 (s, 1H).

MS (ESI⁺) m/z: 484 (MH⁺).

HRMS (ESI⁺) for C₂₇H₂₉F₃N₃O₂ (MH⁺): calcd, 484.22119. found, 484.22093.

### Example 189

### 6-((1-(2-(3,8-Difluoro-6-methoxyquinolin-4-yl)ethyl)-2-oxabicyclo[2.2.2]octan-4-ylamino)methyl)-2H-pyrido[3,2-b][1,4]oxazin-3(4H)-one Hydrochloride

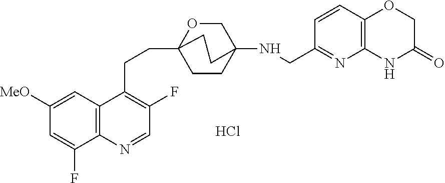 [SMILES: COc1cc(F)c2ncc(F)c(CCC34CCC(NCc5ccc6c(n5)NC(=O)CO6)(CC3)CO4)c2c1 ; Cl]

Step 1

A solution of 2-fluoro-4-methoxyaniline (4.2 g) in toluene (30 mL) was added diethyl ethoxymethylenemalonate (7 g), and the mixture was refluxed for 6 hours. Then the mixture was concentrated and the residue was washed with cold ethanol and dried under reduced pressure to give diethyl 2-((2-fluoro-4-methoxyphenylamino)methylene)malonate (9.2 g). MS m/z: 312 (MH⁺).

Step 2

Diethyl 2-((2-fluoro-4-methoxyphenylamino)methylene)malonate (9.2 g) was added to refluxed diphenyl ether (100 mL) portionwise, and then the solution was refluxed for 20 minutes and was cooled to room temperature. Hexane was added, the brown solid was precipitated out, filtered and washed with hexane, dried under reduced pressure to give ethyl 8-fluoro-6-methoxy-4-oxo-1,4-dihydroquinoline-3-carboxylate (4.8 g). MS m/z: 266 (MH⁺).

Step 3

A solution of ethyl 8-fluoro-6-methoxy-4-oxo-1,4-dihydroquinoline-3-carboxylate (2 g) in N,N-dimethylformamide (20 mL) was added phosphorous tribromide (2.5 g) and the mixture was stirred at room temperature for 3 hours. Then the mixture was poured into ice water, adjusted to pH 9 with aq. sodium hydrogencarbonate, and then was extracted with ethyl acetate. The organic layer was washed with brine, dried, filtered and concentrated. The residue was purified by a CombiFlash® chromatography system (Teledyne Isco, Inc., Lincoln, Nebr.) to give ethyl 4-bromo-8-fluoro-6-methoxyquinoline-3-carboxylate (2.4 g). MS m/z: 328 (MH⁺).

Step 4

To a solution of ethyl 4-bromo-8-fluoro-6-methoxyquinoline-3-carboxylate (2.4 g) in tetrahydrofuran (25 mL) was added a solution of sodium hydroxide (0.56 g in 8 mL of water) slowly. The mixture was stirred overnight at room temperature. Condensed and acidified to pH 5 with concentrated hydrochloric acid. The white precipitate was collected by filtration, washed with water and dried under vacuum to afford pure 4-bromo-8-fluoro-6-methoxyquinoline-3-carboxylic acid (1.6 g). MS m/z: 300 (MH⁺).

Step 5

A mixture of 4-bromo-8-fluoro-6-methoxyquinoline-3-carboxylic acid (500 mg) and N-methyl-2-pyrrolidone (172 mg) in 1,2-dichloroethane (10 mL) was stirred at room temperature for 15 minutes. Diphenyl phosphoryl azide (470 mg) was added dropwise to the clear solution and stirred for 30 minutes then refluxed for another 75 minutes. To the reaction mixture was added tert-butanol (10 mL) and refluxed overnight before cooled down. The reaction mixture was diluted with dichloromethane (100 mL), washed with water and brine and condensed. The residue was purified by column chromatography (20% ethyl acetate in petroleum ether) to give tert-butyl 4-bromo-8-fluoro-6-methoxyquinolin-3-ylcarbamate (300 mg). MS m/z: 371 (MH⁺).

Step 6

To a solution of tert-butyl 4-bromo-8-fluoro-6-methoxyquinolin-3-ylcarbamate (300 mg) in dichloromethane (2 mL) was added trifluoroacetic acid (1 mL) and the mixture was stirred overnight at room temperature and then concentrated. The residue was dissolved in ethyl acetate (50 mL) and washed subsequently with saturated sodium carbonate, water and brine. The ethyl acetate layer was dried over anhydrous sodium sulfate and condensed to give pure 4-bromo-8-fluoro-6-methoxyquinolin-3-amine (200 mg). MS m/z: 271 (MH⁺).

Step 7

To an ice-cooled solution of 4-bromo-8-fluoro-6-methoxyquinolin-3-amine (200 mg) in dry tetrahydrofuran (3 mL) was added nitrosyl tetrafluoroborate (130 mg). The mixture was stirred at 0° C. for 50 minutes then filtrated. The solid cake was washed with cold tetrahydrofuran (1 mL) and dried by vacuum at room temperature to afford a brown powder. This powder was suspended in decaline was heated to 100° C. for 1 hour. Cooled down, diluted with petroleum ether (100 mL) and filtrated through a silica gel pad washed with petroleum ether to remove the decaline then washed with dichloromethane to afford 4-bromo-3,8-difluoro-6-methoxyquinoline as a white solid (80 mg). MS m/z: 274 (MH⁺).

Step 8

To a solution of Intermediate A in anhydrous tetrahydrofuran (3 mL) was added a solution of 9-borabicyclo[3.3.1]nonane dimer (1.2 mL, 0.5 M in tetrahydrofuran) under cooling with ice, the mixture was stirred at room temperature for 1 hour. After quenching the reaction by adding water (1 drop) under cooling, 4-bromo-3,8-difluoro-6-methoxyquinoline (80 mg), tetrakis(triphenylphosphine)palladium (70 mg), tripotassium phosphate (450 mg) and ethanol/water (1 mL, 4:1) was added to the mixture:, and subsequently degassed. The mixture was heated at 70° C. for 18 hours and concentrated in vacuo. After dilution of the residue with ethyl acetate, the mixture was washed with water and brine, dried over anhydrous sodium sulfate, filtered, and then concentrated in vacuo gave crude tert-butyl 1-(2-(3,8-difluoro-6-methoxyquinolin-4-yl)ethyl)-2-oxabicyclo[2.2.2]octan-4-ylcarbamate and used directly. MS m/z: 449 (MH⁺).

Step 9

To a solution of tert-butyl 1-(2-(3,8-difluoro-6-methoxyquinolin-4-yl)ethyl)-2-oxabicyclo[2.2.2]octan-4-ylcarbamate (80 mg) in dichloromethane (2 mL) was added trifluoroacetic acid (1 mL). The mixture was stirred at room temperature for 2 hours, and then concentrated. The residue was dissolved in water, then extracted with ether. The pH of the aqueous layer was adjusted to 10 with sodium carbonate and extracted with ethyl acetate. The organic layer was washed with brine, dried, filtered and concentrated. The residue was used directly in the next step (50 mg). MS m/z: 349 (MH⁺).

Step 10

To a mixture of 1-(2-(3,8-difluoro-6-methoxyquinolin-4-yl)ethyl)-2-oxabicyclo[2.2.2]octan-4-amine (50 mg) and Intermediate I (40 mg) in anhydrous N,N-dimethylformamide (5 mL) was added acetic acid (0.3 mL). The mixture was stirred at room temperature for 10 minutes. Three portions of sodium triacetoxyborohydride (45 mg) was added., then stirred at room temperature overnight. The mixture was concentrated in vacuo.

After dilution of the residue with dichloromethane, the mixture was washed with saturated sodium carbonate solution, water and brine. The organic extracts were dried over anhydrous sodium sulfate, filtered, and then concentrated in vacuo. The residue was purified via prep-TLC (dichloromethane:methanol=10:1) of the residue and gave 6-((1-(2-(3,8-difluoro-6-methoxyquinolin-4-yl)ethyl)-2-oxabicyclo[2.2.2]octan-4-ylamino)methyl)-2H-pyrido[3,2-b][1,4]oxazin-3(4H)-one (20 mg). MS m/z: 511 (MH⁺).

Step 11

To a solution of 6-((1-(2-(3,8-difluoro-6-methoxyquinolin-4-yl)ethyl)-2-oxabicyclo[2.2.2]octan-4-ylamino)methyl)-2H-pyrido[3,2-b][1,4]oxazin-3(4H)-one (20 mg) in dichloromethane (2 mL) and ethanol (0.5 mL) was added a solution of hydrogen chloride (10 uL, 4 M in 1,4-dioxane) under cooling with ice. The mixture was stirred at room temperature for 2 hours and concentrated in vacuo. Treatment of the residue with ethanol gave the title compound (20 mg).

¹H NMR (MeOD): δ 1.71-1.75 (m, 2H), 1.85-2.15 (m, 8H), 3.12-3.16 (m, 2H), 3.93-3.96 (m, 6H), 4.66 (s, 2H), 7.03 (d, J=8.0 Hz, 1H), 7.13-7.17 (m, 1H), 7.25 (s, 1H), 7.31 (d, J=8.4 Hz, 1H), 8.56 (s, 1H).

MS m/z: 511 (MH⁺).

### Example 190

### 6-((1-(1-(3-Fluoro-6-methoxy-1,5-naphthyridin-4-yl)-2-hydroxypropan-2-yl)-2-oxabicyclo[2.2.2]octan-4-ylamino)methyl)-2H-pyrido[3,2-b][1,4]oxazin-3(4H)-one Hydrochloride

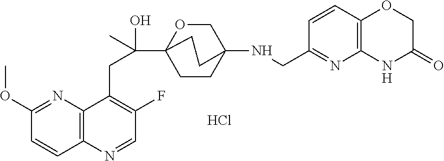 [SMILES: COc1ccc2ncc(F)c(CC(C)(O)C34CCC(NCc5ccc6c(n5)NC(=O)CO6)(CC3)CO4)c2n1 ; Cl]

Step 1

To a solution of Intermediate F (383 mg) in tetrahydrofuran (5 mL) was added a solution of methylmagnesium bromide (1 mL, 3.0 M in ether) at −70° C. The mixture was stirred at −70° C. for 30 minutes then warmed to room temperature. To the reaction mixture was added saturated ammonium chloride solution and the mixture was extracted with ethyl acetate twice. The organic layer was concentrated and the residue was purified by prep-TLC (petroleum ether:ethyl acetate=5:1) to afford a white solid tert-butyl 1-(1-hydroxyethyl)-2-oxabicyclo[2.2.2]octan-4-ylcarbamate (120 mg).

¹H NMR (CDCl₃): δ 0.98 (d, J=6.4 Hz, 3H), 1.36 (s, 9H), 1.69-1.78 (m, 4H), 1.92-2.07 (m, 4H), 3.57 (d, J=6.4 Hz, 1H), 3.93 (s, 2H), 4.23 (s, 1H).

Step 2

A suspension of tert-butyl 1-(1-hydroxyethyl)-2-oxabicyclo[2.2.2]octan-4-ylcarbamate (120 mg) and Dess-Martin periodinane (940 mg) was stirred overnight at room temperature. The solid was collected by filtration and then washed with dichloromethane. The filtrate was concentrated and the residue was purified by prep-TLC (petroleum ether:ethyl acetate=3:1) to afford tert-butyl 1-acetyl-2-oxabicyclo[2.2.2]octan-4-ylcarbamate as a white solid (54 mg).

¹H NMR (CDCl₃): δ 1.40 (s, 9H), 1.79-1.86 (m, 2H), 1.90-1.98 (m, 2H), 2.04-2.11 (m, 2H), 2.17 (s, 3H), 4.00 (s, 2H).

Step 3

To a −78° C. solution of Intermediate R (77 mg) in tetrahydrofuran (3 mL) was added a solution of lithium diisopropyl amide (0.2 mL, 2.0 M in tetrahydrofuran) in dropwise fashion and stirred for 15 minutes. To this mixture was added dropwise a solution of tert-butyl 1-acetyl-2-oxabicyclo[2.2.2]octan-4-ylcarbamate (54 mg) in tetrahydrofuran (1 mL). The resulting mixture was stirred at −78° C. for 30 minutes then warmed to room temperature. The reaction was quenched by the addition of saturated ammonium chloride solution and extracted twice with ethyl acetate. The organic layer was concentrated and the residue was purified by prep-TLC (petroleum ether:ethyl acetate=3:1) to afford a white solid tert-butyl 1-(1-(3-fluoro-6-methoxy-1,5-naphthyridin-4-yl)-2-hydroxypropan-2-yl)-2-oxabicyclo[2.2.2]octan-4-ylcarbamate (37 mg). MS m/z: 462 (MH⁺).

Step 4

To a solution of tert-butyl 1-(1-(3-fluoro-6-methoxy-1,5-naphthyridin-4-yl)-2-hydroxypropan-2-yl)-2-oxabicyclo[2.2.2]octan-4-ylcarbamate (37 mg) in dichloromethane (1 mL) was added trifluoroacetic acid (1 mL). The mixture was stirred at room temperature for 30 minutes and concentrated under vacuum. After dilution of the residue with water, the mixture was washed with methyl t-butyl ether twice. The aqueous layer was adjusted to pH 13 by addition of aqueous sodium carbonate solution and extract twice with ethyl acetate. The combined organic layer was washed with brine, dried over anhydrous sodium sulfate and concentrated to give pure 2-(4-amino-2-oxabicyclo[2.2.2]octan-1-yl)-1-(3-fluoro-6-methoxy-1,5-naphthyridin-4-yl)propan-2-ol (25 mg). MS m/z: 362 (MH⁺).

Step 5

To a mixture of 2-(4-amino-2-oxabicyclo[2.2.2]octan-1-yl)-1-(3-fluoro-6-methoxy-1,5-naphthyridin-4-yl)propan-2-ol (25 mg) and Intermediate I (20 mg) in anhydrous N,N-dimethylformamide (3.5 mL) was added acetic acid (0.5 mL). The mixture was stirred at room temperature for 30 minutes followed by addition of the portions of sodium triacetoxyborohydride (42 mg). Then, the mixture was stirred at room temperature overnight, then concentrated in vacuo. After dilution of the residue with dichloromethane, the mixture was washed with saturated sodium carbonate solution, water and brine. The organic extracts were dried over anhydrous sodium sulfate and then concentrated in vacuo. The residue was purified by prep-TLC (dichloromethane:methanol=10:1) to give 6-((1-(1-(3-fluoro-6-methoxy-1,5-naphthyridin-4-yl)-2-hydroxypropan-2-yl)-2-oxabicyclo[2.2.2]octan-4-ylamino)methyl)-2H-pyrido[3,2-b][1,4]oxazin-3(4H)-one (21 mg). MS m/z: 524 (MH⁺).

Step 6

To a solution of 6-((1-(1-(3-fluoro-6-methoxy-1,5-naphthyridin-4-yl)-2-hydroxypropan-2-yl)-2-oxabicyclo[2.2.2]octan-4-ylamino)methyl)-2H-pyrido[3,2-b][1,4]oxazin-3(4H)-one (21 mg) in dichloromethane (2 mL) and ethanol (0.5 mL) was added a solution of hydrogen chloride (10 uL, 4 M in 1,4-dioxane) under cooling with ice. The mixture was stirred at room temperature for 2 hours and concentrated in vacuo. Treatment of the residue with ethanol gave the title compound.

¹H NMR (MeOD): δ 1.02 (s, 3H), 2.01-2.29 (m, 6H), 2.37-2.42 (m, 2H), 3.60 (d, J=12.8 Hz, 1H), 3.80 (d, J=12.8 Hz, 1H), 4.08 (s, 2H), 4.16 (s, 3H), 4.24 (s, 2H), 4.68 (s, 2H), 7.12 (d, J=8.0 Hz, 1H), 7.36 (d, J=8.0 Hz, 1H), 7.42 (d, J=7.2 Hz, 1H), 8.37 (d, J=7.2 Hz, 1H), 9.05 (s, 1H).

MS m/z: 524 (MH⁺).

### Example 191

### 6-((1-(2-(3-Fluoro-6-methoxy-8-methyl-1,5-naphthyridin-4-yl)ethyl)-2-oxabicyclo[2.2.2]octan-4-ylamino)methyl)-2H-pyrido[3,2-b][1,4]oxazin-3(4H)-one Hydrochloride

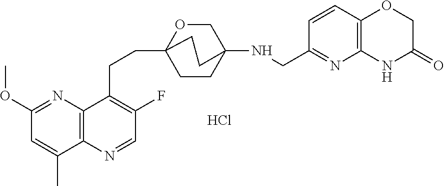 [SMILES: COc1cc(C)c2ncc(F)c(CCC34CCC(NCc5ccc6c(n5)NC(=O)CO6)(CC3)CO4)c2n1 ; Cl]

Step 1

A mixture of 6-methoxy-4-methylpyridin-3-amine (4.1 g), 2,2-dimethyl-1,3-dioxane-4,6-dione (5.1 g) and triethyl orthoformate (4.8 g) in ethanol (15 mL) was refluxed for 2 hours and then cooled down to room temperature. The precipitate was collected by filtration and washed with cold ethanol, dried under vacuum to give 5-((6-methoxy-4-methylpyridin-3-ylamino)methylene)-2,2-dimethyl-1,3-dioxane-4,6-dione.

Step 2

5((6-Methoxy-4-methylpyridin-3-ylamino)methylene)-2,2-dimethyl-1,3-dioxane-4,6-dione was added portionwise to diphenyl ether (10 mL) at 260° C. and refluxed for 10 minutes. The mixture was cooled to 60° C. and diluted with petroleum ether. The resulting precipitates were collected by filtration and washed with petroleum ether to give crude 6-methoxy-8-methyl-1,5-naphthyridin-4-ol (3.2 g). MS m/z: 191 (MH⁺).

Step 3

6-Methoxy-8-methyl-1,5-naphthyridin-4-ol (190 mg) was added slowly to fuming nitric acid (2 mL) at 0° C. The mixture was heated to 90° C. for 2 hours before being poured into ice water (20 mL). The pH was adjusted to 5-6 with saturated sodium carbonate solution. The yellow precipitate was collected by filtration and washed with water. The 6-methoxy-8-methyl-3-nitro-1,5-naphthyridin-4-ol, obtained as a wet cake, was dried and used directly.

¹H NMR (DMSO-d₆): δ 2.51 (s, 3H), 3.93 (s, 3H), 7.16 (s, 1H), 8.80 (s, 1H).

MS m/z: 236 (MH⁺).

Step 4

To a suspension of 6-methoxy-8-methyl-3-nitro-1,5-naphthyridin-4-ol (143 mg) in N,N-dimethylformamide (5 mL) was added phosphorous tribromide (198 mg) while cooling with water. The mixture was stirred overnight at room temperature then poured into ice water, the mixture was adjusted to pH 8 by addition of saturated sodium hydrogencarbonate solution. The resulting precipitates were collected by filtration, washed with water and dried to give 8-bromo-2-methoxy-4-methyl-7-nitro-1,5-naphthyridine (163 mg). MS m/z: 299 (MH⁺).

Step 5

A suspension of 8-bromo-2-methoxy-4-methyl-7-nitro-1,5-naphthyridine (163 mg), iron powder (200 mg) and solid ammonium chloride (200 mg) in ethanol (8 mL) and water (2 mL) was refluxed for 2 hours. The reaction mixture was filtered. The resulting solids were washed with hot ethyl acetate, then water and the ethyl acetate layer was separated. The water layer was extracted with ethyl acetate twice. The combined organic layer was washed with brine and filtered though a silica gel pad then concentrated to give pure 4-bromo-6-methoxy-8-methyl-1,5-naphthyridin-3-amine (105 mg). MS m/z: 269 (MH⁺).

Step 6

To an ice-cooled solution of 4-bromo-6-methoxy-8-methyl-1,5-naphthyridin-3-amine (105 mg) in dry tetrahydrofuran (5 mL) was added nitrosyl tetrafluoroborate (54 mg). The mixture was stirred at 0° C. for 50 minutes then filtered. The solid cake was washed with cold tetrahydrofuran (1 mL) and dried by vacuum at room temperature to afford a brown powder. The powder was suspended in decaline and heated to 100° C. for 1 hour, then allowed to cool down to room temperature. The mixture was diluted with petroleum ether (100 mL) and filtered through a silica gel pad washed with petroleum ether to remove the decaline then washed with dichloromethane to afford 8-bromo-7-fluoro-2-methoxy-4-methyl-1,5-naphthyridine as a white solid (80 mg).

¹H NMR (CDCl₃): δ 2.65 (d, J=1.2 Hz, 3H), 4.06 (s, 3H), 6.91 (s, 1H), 8.54 (s, 1H).

MS m/z: 272 (MH⁺).

Step 7

To a solution of Intermediate B (100 mg) in anhydrous tetrahydrofuran (1.8 mL) was added a solution of 9-borabicyclo[3.3.1]nonane dimer (1.6 mL, 0.5 M in tetrahydrofuran) under cooling with ice. The mixture was stirred at room temperature for 1 hour. After quenching the reaction by adding water (1 drop) under cooling, 8-bromo-7-fluoro-2-methoxy-4-methyl-1,5-naphthyridine (80 mg), tetrakis(triphenylphosphine)palladium (91.2 mg), tripotassium phosphate (0.6 g) and ethanol/water (2 mL, 4:1) was added to the mixture and degassed. The mixture was heated at 70° C. for 12 hours and concentrated in vacuo. After dilution of the residue with ethyl acetate, the mixture was washed with water and brine, dried over anhydrous sodium sulfate, filtered, and then concentrated in vacuo gave crude tert-butyl 1-(2-(3-fluoro-6-methoxy-8-methyl-1,5-naphthyridin-4-yl)ethyl)-2-oxabicyclo[2.2.2]octan-4-ylcarbamate and used directly.

Step 8

To a solution of tert-butyl 1-(2-(3-fluoro-6-methoxy-8-methyl-1,5-naphthyridin-4-yl)ethyl)-2-oxabicyclo[2.2.2]octan-4-ylcarbamate (130 mg crude) in dichloromethane (2 mL) was added trifluoroacetic acid (2 mL) and the mixture was stirred at room temperature for 30 minutes and concentrated in vacuo. After dilution of the residue with water, the mixture was washed with methyl tert-butyl ether twice. The aqueous layer was adjusted to pH 13 by addition of aqueous sodium carbonate solution and extracted twice with ethyl acetate. The combined ethyl acetate layer was washed with brine, dried over anhydrous sodium sulfate and condensed to give pure 1-(2-(3-fluoro-6-methoxy-8-methyl-1,5-naphthyridin-4-yl)ethyl)-2-oxabicyclo[2.2.2]octan-4-amine. MS m/z: 347 (MH⁺).

Step 9

To a mixture of 1-(2-(3-fluoro-6-methoxy-8-methyl-1,5-naphthyridin-4-yl)ethyl)-2-oxabicyclo[2.2.2]octan-4-amine (95 mg) and Intermediate I (76 mg) in anhydrous N,N-dimethylformamide (3.5 mL) was added acetic acid (0.5 mL). The mixture was stirred at room temperature for 30 minutes followed by addition of three portions of sodium triacetoxyborohydride (89 mg). The mixture was stirred at room temperature overnight, then concentrated in vacuo. After dilution of the residue with dichloromethane, the mixture was washed with saturated sodium carbonate solution, water and brine. The organic extracts were dried over anhydrous sodium sulfate then concentrated in vacuo. The residue was purified by prep-TLC (dichloromethane:methanol=10:1) to gave 6-((1-(2-(3-fluoro-6-methoxy-8-methyl-1,5-naphthyridin-4-yl)ethyl)-2-oxabicyclo[2.2.2]octan-4-ylamino)methyl)-2H-pyrido[3,2-b][1,4]oxazin-3(4H)-one (30 mg). MS m/z: 508 (MH⁺).

Step 10

To a solution of 6-((1-(2-(3-fluoro-6-methoxy-8-methyl-1,5-naphthyridin-4-yl)ethyl)-2-oxabicyclo[2.2.2]octan-4-ylamino)methyl)-2H-pyrido[3,2-b][1,4]oxazin-3(4H)-one (30 mg) in dichloromethane (2 mL) and ethanol (0.5 mL) was added a solution of hydrogen chloride (11 μL 4 M in 1,4-dioxane) under cooling with ice. The mixture was stirred at room temperature for 2 hours and concentrated in vacuo. Treatment of the residue with ethanol gave the title compound.

¹H NMR (CD₃OD): δ 1.71-1.76 (m, 2H), 1.80-1.88 (m, 6H), 2.00-2.02 (m, 2H), 2.66 (s, 3H), 3.76 (s, 4H), 4.04 (s, 3H), 4.58 (s, 2H), 4.62 (s, 2H), 6.95-6.99 (m, 2H), 7.25 (d, J=8.0 Hz, 1H), 8.56 (s, 1H).

MS m/z: 508 (MH⁺).

### Example 192

The following compound was prepared consistent with the methods described herein.

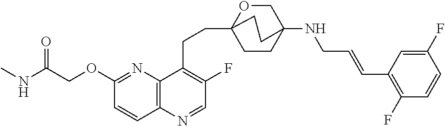 [SMILES: CNC(=O)COc1ccc2ncc(F)c(CCC34CCC(NC/C=C/c5cc(F)ccc5F)(CC3)CO4)c2n1]

### (E)-2-(8-(2-(4-(3-(2,5-Difluorophenyl)allylamino)-2-oxabicyclo[2.2.2]octan-1-yl)ethyl)-7-fluoro-1,5-naphthyridin-2-yloxy)-N-methylacetamide

¹H NMR (DMSO-d₆) δ 1.51-1.77 (m, 10H), 1.78-1.91 (m, 1H), 2.62 (d, J=4.9 Hz, 3H), 2.94-3.09 (m, 2H), 3.16-3.58 (m, 2H), 3.59 (s, 2H), 4.89 (s, 2H), 6.40-6.52 (m, 1H), 6.59 (d, J=17.0 Hz, 1H), 7.04-7.13 (m, 1H), 7.16-7.27 (m, 1H), 7.30 (d, J=9.2 Hz, 1H), 7.39-7.49 (m, 1H), 7.95-8.04 (m, 1H), 8.31 (d, J=9.2 Hz, 1H), 8.76 (s, 1H).

MS (ESI⁺) m/z: 541 (MH⁺).

HRMS (ESI⁺) for C₂₉H₃₂F₃N₄O₃ (MH⁺): calcd, 541.24265. found, 541.24357.

### Example 193

The following compound was prepared consistent with the methods described herein.

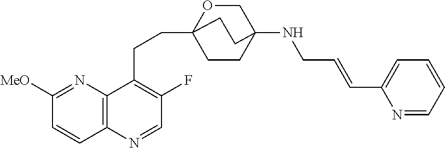 [SMILES: COc1ccc2ncc(F)c(CCC34CCC(NC/C=C/c5ccccn5)(CC3)CO4)c2n1]

### (E)-1-(2-(3-Fluoro-6-methoxy-1,5-naphthyridin-4-yl)ethyl)-N-(3-(pyridin-2-yl)allyl)-2-oxabicyclo[2.2.2]octan-4-amine

¹H NMR (DMSO-d₆) δ 1.58-1.78 (m, 9H), 1.80-1.92 (m, 2H), 3.08-3.16 (m, 2H), 3.33 (brs, 2H), 3.59 (brs, 2H), 4.03 (s, 3H), 6.56 (d, J=15.9 Hz, 1H), 6.71 (td, J=15.9, 5.5 Hz, 1H), 7.18 (ddd, J=7.3, 4.9, 1.2 Hz, 1H), 7.21 (d, J=9.2 Hz, 1H), 7.38 (d, J=7.3 Hz, 1H), 7.70 (td, J=7.3, 1.8 Hz, 1H), 8.26 (d, J=9.2 Hz, 1H), 8.48 (dd, J=4.9, 1.2 Hz, 1H), 8.74 (s, 1H).

MS (ESI) m/z: 449 (MH⁺).

HRMS (ESI⁺) for C₂₆H₃₀FN₄O₂ (MH⁺): calcd, 449.23528. found 449.23481.

### Example 194

### 1-(2-(3-Fluoro-6-methoxy-1,5-naphthyridin-4-yl)-3-hydroxypropyl)-N-((3-oxo-3,4-dihydro-2H-pyrido[3,2-b][1,4]oxazin-6-yl)methyl)-2-oxabicyclo[2.2.2]octan-4-aminium chloride

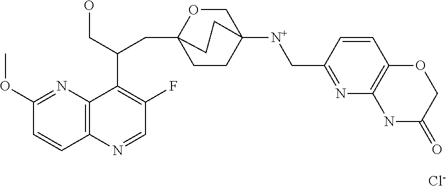 [SMILES: COc1ccc2ncc(F)c(C(CO)CC34CCC([NH2+]Cc5ccc6c(n5)NC(=O)CO6)(CC3)CO4)c2n1 ; [Cl-]]

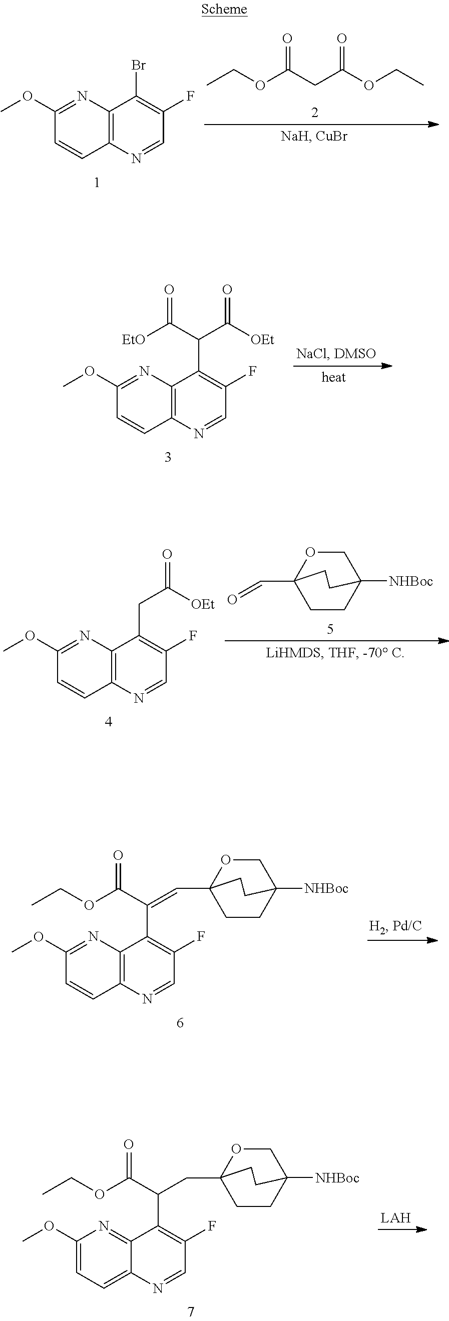

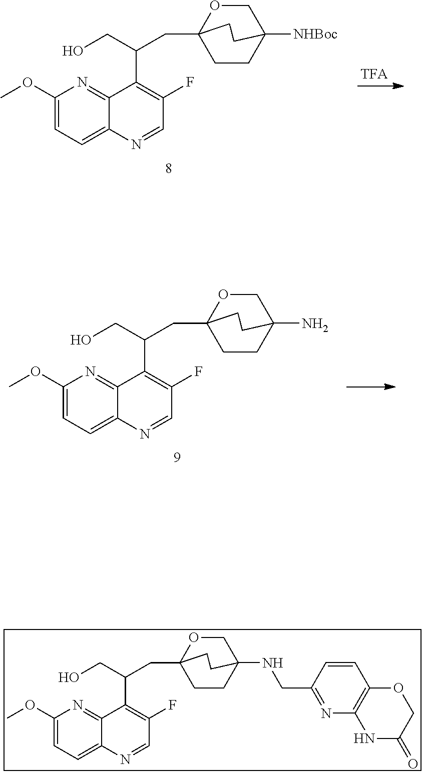 [SMILES: COc1ccc2ncc(F)c(C(CO)CC34CCC(NC(=O)OC(C)(C)C)(CC3)CO4)c2n1 ; COc1ccc2ncc(F)c(C(CO)CC34CCC(N)(CC3)CO4)c2n1 ; COc1ccc2ncc(F)c(C(CO)CC34CCC(NCc5ccc6c(n5)NC(=O)CO6)(CC3)CO4)c2n1]

### Preparation of Compound 3

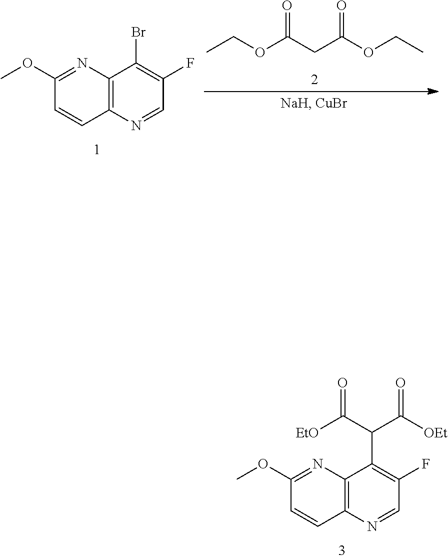

Diethyl malonate (3.8 g, 24 mmol) was added to a suspension of NaH (0.9 g, 23 mmol, 60 percent in mineral oil) in 40 mL of dioxane. The mixture was stirred at room temperature for 5 min and then heated at 80° C. for 15 minutes. CuBr (0.4 g, 2.8 mmol) and 1 (2.1 g, 8 mmol) were added. The mixture was refluxed for 3 hours before cooled down. The mixture was diluted with EtOAc and washed with aq. NH₄Cl and brine, dried over Na₂SO₄, filtered and concentrated to dryness. The residue was purified by column chromatography (PE/EtOAc=10:1) to afford a yellow oil 3 (1.8 g, yield 67%). ¹H-NMR (400 MHz, CDCl₃) δ ppm 8.70 (s, 1H), 8.19 (d, J=9.2 Hz, 1H), 7.08 (d, J=9.2 Hz, 1H), 5.78 (s, 1H), 4.22 (t, J=7.2 Hz, 4H), 1.22 (t, J=7.8 Hz, 6H). MS m/z 337 (M+1)⁺.

### Preparation of Compound 4

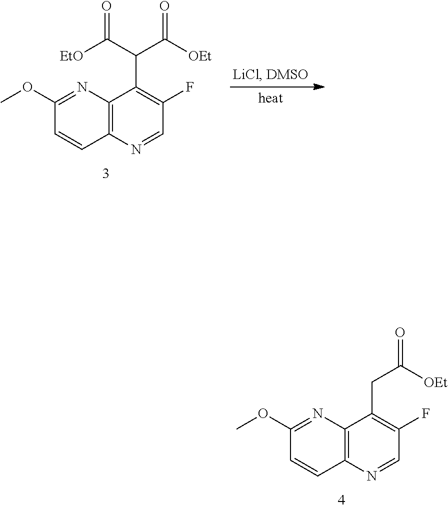 [SMILES: CCOC(=O)C(C(=O)OCC)c1c(F)cnc2ccc(OC)nc12 ; CCOC(=O)Cc1c(F)cnc2ccc(OC)nc12]

A solution of 3 (1.8 g, 5.4 mmol) in 20 mL of DMSO was added water (117 mg, 6.5 mmol) and LiCl (964 mg, 22.7 mmol). The mixture was stirred at 110° C. for 18 hours before cooled down and diluted with EtOAc. The mixture was washed with water and brine, dried over Na₂SO₄, filtered and concentrated to dryness. The residue was purified by column chromatography (PE/EtOAc=10:1) to afford a colorless oil 4 (1.1 g, yield 79%). MS m/z 265 (M+1)⁺.

### Preparation of Compound 6

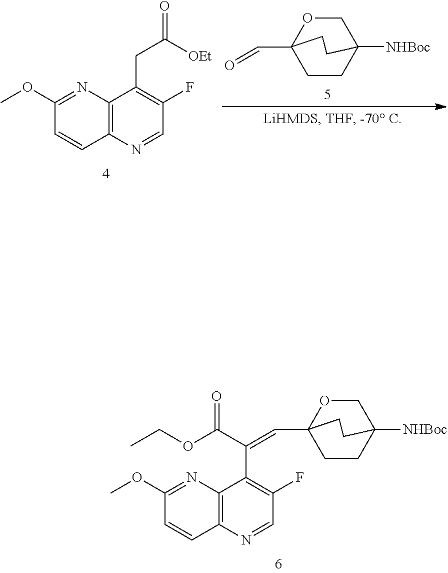

A solution of 4 (528 mg, 2 mmol) in 5 mL of THF was added LiHMDS (2 mL, 2.0 mmol) dropwise at −30° C. and stirred for 1 hour then a solution of 5 (255 mg, 1 mmol in 2 mL of THF) was added slowly. The mixture was stirred at −30° C. for 30 minutes and then warmed to room temperature for 2 hours. Quenched with saturated NH₄Cl and extracted with EtOAc twice. Dried and concentrated, the residue was purified by column chromatography (PE/EtOAc=3:1) to afford pure 6 (240 mg, yield 48%). MS m/z 502 (M+1)⁺.

### Preparation of Compound 7

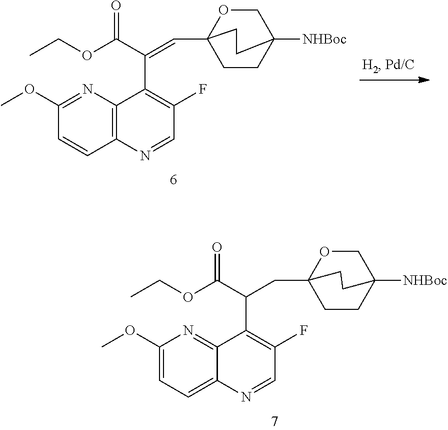 [SMILES: CCOC(=O)/C(=C/C12CCC(NC(=O)OC(C)(C)C)(CC1)CO2)c1c(F)cnc2ccc(OC)nc12 ; CCOC(=O)C(CC12CCC(NC(=O)OC(C)(C)C)(CC1)CO2)c1c(F)cnc2ccc(OC)nc12]

To a solution of 6 (270 mg, 0.54 mmol) in EtOAc (20 mL) was added PdC (100 mg, 10%). The mixture was stirred at 40° C. for 1.5 hours. Filtered and concentrated in vacuo, the product was obtained as a solid (210 mg, 77.5%). MS m/z 504 (M+1)⁺.

### Preparation of Compound 8

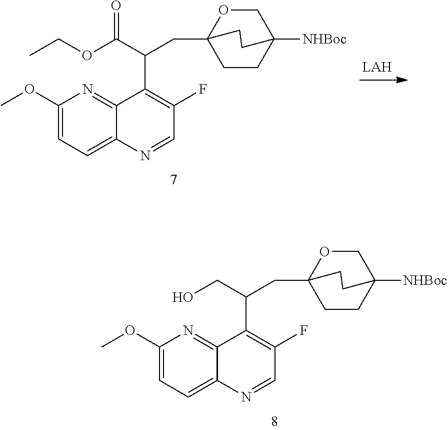 [SMILES: CCOC(=O)C(CC12CCC(NC(=O)OC(C)(C)C)(CC1)CO2)c1c(F)cnc2ccc(OC)nc12 ; COc1ccc2ncc(F)c(C(CO)CC34CCC(NC(=O)OC(C)(C)C)(CC3)CO4)c2n1]

To a solution of 7 (150 mg, 0.3 mmol) in THF (10 mL) was added LiAlH₄ (20 mg, 0.53 mmol). The mixture was stirred at room temperature for 1.5 hours. After quenching with saturated ammonium chloride solution, the mixture was extracted with EtOAc twice. The organic layers were dried and concentrated to give the crude 8 (50 mg, 36.2%). MS m/z 462 (M+1)⁺.

### Preparation of Compound 9

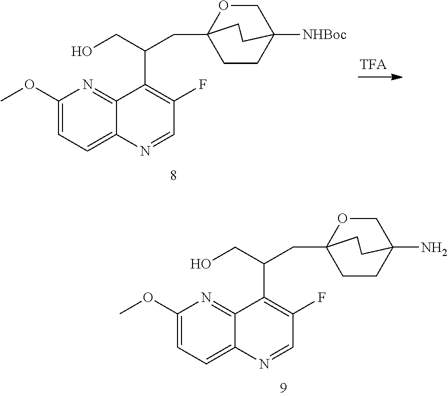 [SMILES: COc1ccc2ncc(F)c(C(CO)CC34CCC(NC(=O)OC(C)(C)C)(CC3)CO4)c2n1 ; COc1ccc2ncc(F)c(C(CO)CC34CCC(N)(CC3)CO4)c2n1]

To a solution of 8 (50 mg, 0.11 mmol) in DCM (2 mL) was added TFA (5 mL). The mixture was stirred at room temperature for overnight. The reaction solution was concentrated and then the NaHCO₃ solution was added. The mixture was extracted with DCM/MeOH (10:1). The organic extracts were dried over anhydrous sodium sulfate, filtered, and then concentrated in vacuo to give the crude 9 (30 mg, 76.5%). MS m/z 362 (M+1)⁺.

### Preparation of Example 194

A mixture of 9 (30 mg, 0.08 mmol) and pyridoxazinecarbaldehyde (50 mg, 0.28 mmol) in anhydrous DMF (3.5 mL) was added acetic acid (0.5 mL) was stirred at room temperature for 30 minutes. The resulting solution was added three times of sodium triacetoxyborohydride (50 mg, 0.25 mmol) and stirred at room temperature for overnight. The mixture was concentrated in vacumm. After diluted with dichloromethane, the mixture was washed with saturated sodium carbonate solution, water and brine. The organic extracts were dried over anhydrous Na₂SO₄ then concentrated in vacumm. The residue was purified by prep-TLC (DCM/MeOH=10:1) to afford a solid Example 194. ¹H-NMR (400 MHz, MeOD) δ ppm 8.65 (s, 1H), 8.25 (d, J=8.8 Hz, 1H), 7.45 (d, J=9.6 Hz, 1H), 7.2 (d, J=8.0 Hz, 1H), 7.16 (d, J=8.0 Hz, 1H), 4.64 (s, 2H), 4.19 (s, 3H), 4.00 (m, 1H), 3.65 (s, 2H), 3.35 (s, 1H), 3.25 (s, 1H), 1.95 (m, 2H), 1.75-2.1 (m, 8H),. MS m/z 524 (M+1)⁺.

### Example 195

### Sodium 2-(3-fluoro-6-methoxy-1,5-naphthyridin-4-yl)-3-(4-(((3-oxo-3,4-dihydro-2H-pyrido[3,2-b][1,4]oxazin-6-yl)methyl)amino)-2-oxabicyclo[2.2.2]octan-1-yl)propanoate

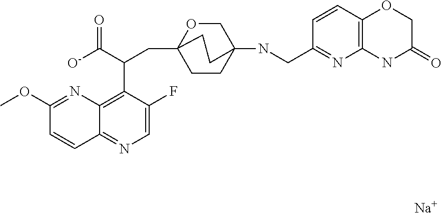 [SMILES: COc1ccc2ncc(F)c(C(CC34CCC(NCc5ccc6c(n5)NC(=O)CO6)(CC3)CO4)C(=O)[O-])c2n1 ; [Na+]]

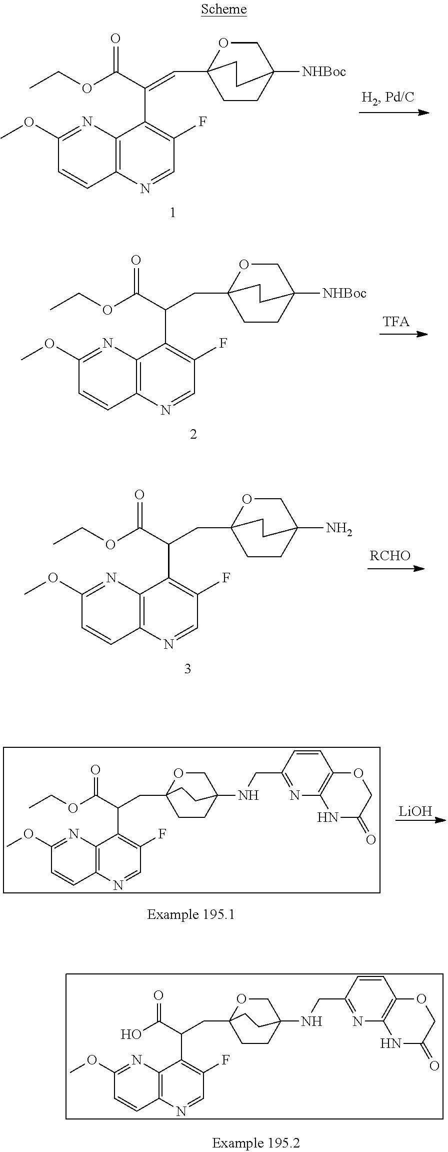

### Preparation of Compound 2

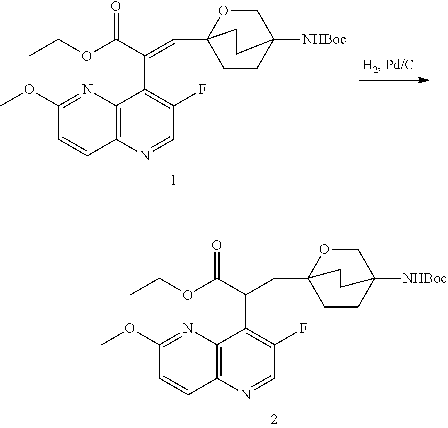 [SMILES: CCOC(=O)/C(=C/C12CCC(NC(=O)OC(C)(C)C)(CC1)CO2)c1c(F)cnc2ccc(OC)nc12 ; CCOC(=O)C(CC12CCC(NC(=O)OC(C)(C)C)(CC1)CO2)c1c(F)cnc2ccc(OC)nc12]

To a solution of 1 (215 mg, 0.43 mmol) in EtOAc (20 mL) was added PdC (100 mg, 10%) and the mixture was stirred at 40° C. for 1.5 hours. After filtered, the mxitrue was concentrated in vacuo to give the crude 2 (210 mg, 96.8%). MS m/z 504.5 (M+1)⁺.

### Preparation of Compound 3

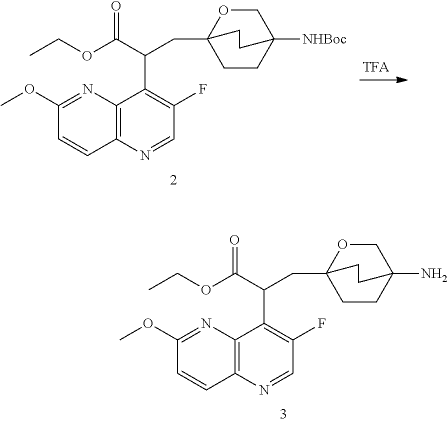 [SMILES: CCOC(=O)C(CC12CCC(NC(=O)OC(C)(C)C)(CC1)CO2)c1c(F)cnc2ccc(OC)nc12 ; CCOC(=O)C(CC12CCC(N)(CC1)CO2)c1c(F)cnc2ccc(OC)nc12]

To a solution of 2 (210 mg, 0.432 mmol) in DCM (2 mL) was added TFA (10 mL). The mixture was stirred at room temperature overnight. The reaction solution was concentrated and then the NaHCO₃ solution was added. The mixture was extracted with ethyl acetate. The organic extracts were washed with water, dried over anhydrous sodium sulfate, filtered, and then concentrated in vacuo to give the crude 3 (120 mg, 69.2%). MS m/z 404.5 (M+1)⁺.

### Preparation of Example 195.1

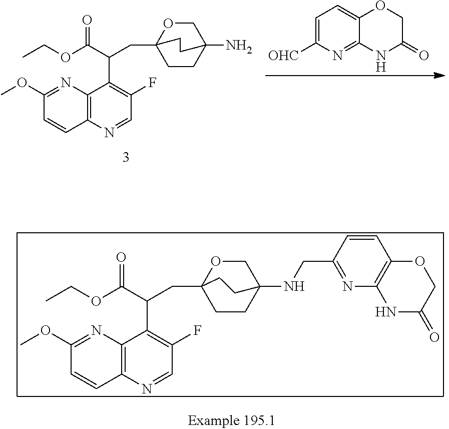 [SMILES: CCOC(=O)C(CC12CCC(N)(CC1)CO2)c1c(F)cnc2ccc(OC)nc12 ; O=Cc1ccc2c(n1)NC(=O)CO2 ; CCOC(=O)C(CC12CCC(NCc3ccc4c(n3)NC(=O)CO4)(CC1)CO2)c1c(F)cnc2ccc(OC)nc12]

A mixture of 3 (120 mg, 0.3 mmol) and pyridoxazinecarbaldehyde (150 mg, 0.83 mmol) in anhydrous DMF (3.5 mL) was added acetic acid (0.5 mL) was stirred at room temperature for 30 minutes. The resulting solution was added three times of sodium triacetoxyborohydride (210 mg, 1 mmol) and stirred at room temperature overnight. The mixture was concentrated in vacumm. After dilution of the residue with dichloromethane, the mixture was washed with saturated sodium carbonate solution, water and brine. The organic extracts were dried over anhydrous Na₂SO₄ then concentrated in vacumm. The residue was purified by prep-TLC (DCM/MeOH=10:1) to afford a solid Example 195.1. ¹H-NMR (400 MHz, MeOD) δ ppm 8.65 (s, 1H), 8.25 (d, J=8.8 Hz, 1H), 7.45 (d, J=9.6 Hz, 1H), 7.2 (d, J=8.0 Hz, 1H), 7.16 (d, J=8.0 Hz, 1H), 5.7 (d, J=9.6 Hz, 1H), 4.64 (s, 2H), 4.19 (s, 3H), 4.00 (s, 2H), 3.85 (s, 2H), 2.25 (m, 2H), 1.75-2.1 (m, 8H), 1.05-1.1 (m, 2H). MS m/z 566.5 (M+1)⁺.

### Preparation of Example 195.2

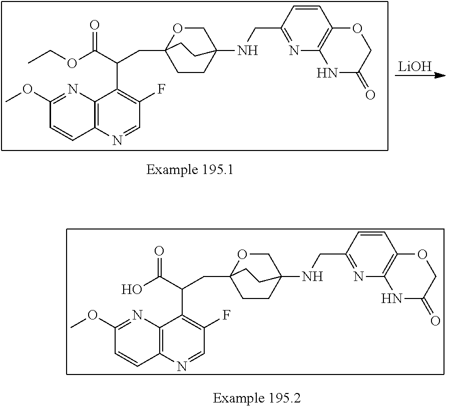

A solution of Example 195.1 (100 mg, 0.177 mmol) in 10 mL of THF/MeOH/H₂O (2:2:1) was added LiOH.H₂O (84 mg, 2 mmol) at room temperature. The mixture was stirred overnight, diluted with water and washed with MTBE twice. The water layer was acidified to pH=5 with hydrochloric acid then extracted with DCM and MeOH (10:1). The organic layer was washed with brine, dried over anhydrous Na₂SO₄ and condensed. The residue was purified by prep-HPLC and the desired solution was lyophilized to get solid, which was converted to sodium salt with 1 N NaOH. The resulting solid was washed with DCM and MeOH (10:1) to give a white solid Example 195.2. ¹H-NMR (400 MHz, MeOD) δ ppm 8.65 (s, 1H), 8.25 (d, J=8.8 Hz, 1H), 7.45 (d, J=9.6 Hz, 1H), 7.2 (d, J=8.0 Hz, 1H), 6.85 (d, J=8.4 Hz, 1H), 4.78 (d, J=8.0 Hz, 1H), 4.65 (s, 2H), 4.15 (s, 3H), 3.8 (s, 2H), 3.5 (m, 2H), 2.65 (d, J=9 Hz, 1H), 2.25 (m, 1H), 1.65-1.9 (m, 8H). MS m/z 538.5 (M+1)⁺.

### Example 196

### 7-Chloro-N-(4-(2-(3-fluoro-6-methoxy-1,5-naphthyridin-4-yl)ethyl)bicyclo[2.2.2]octan-1-yl)-3-oxo-3,4-dihydro-2H-pyrido[3,2-b][1,4]thiazine-6-carboxamide

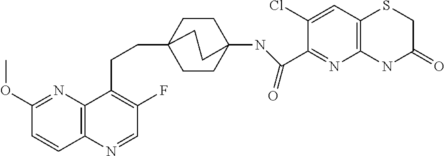 [SMILES: COc1ccc2ncc(F)c(CCC34CCC(NC(=O)c5nc6c(cc5Cl)SCC(=O)N6)(CC3)CC4)c2n1]

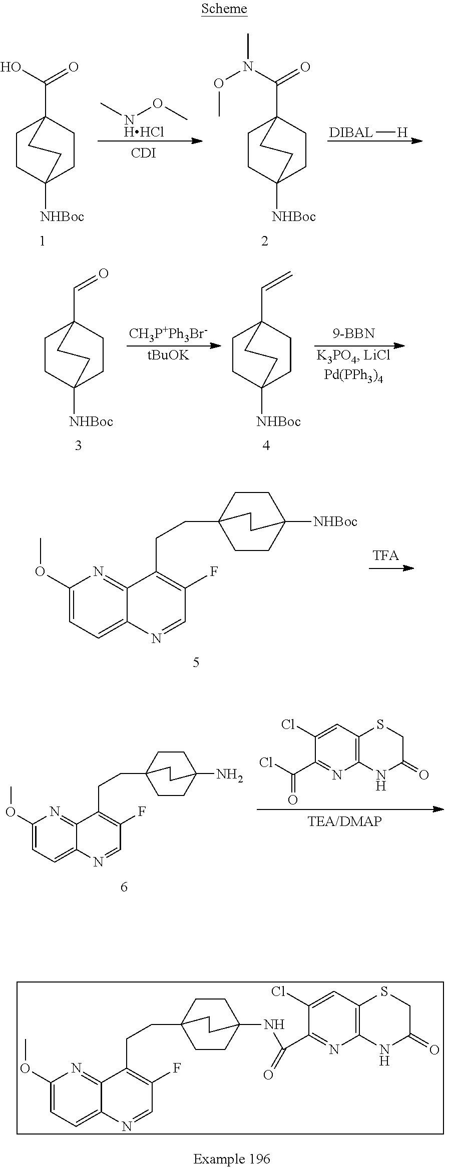

### Preparation of Compound 2

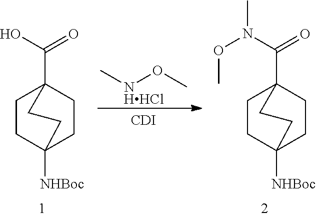

To a solution of 1 (1.07 g, 4.0 mmol, 1.0 eq) in DMF (15 mL) was added CDI (773 mg, 4.8 mmol, 1.2 eq) and then kept stirred for 1 h, and then N,O-dimethylhydroxylamine hydrochloride (463 mg, 4.8 mmol, 1.2 eq) was added. The mixture was stirred at r.t. overnight before partitioned between water and EtOAc. The organic layers were washed with brine, dried over sodium sulfate and concentrated. The residue was purified by flash-chromatography to give 2 (960 mg, 77.4%). ¹H-NMR (400 MHz, MeOH-d4) δ ppm 3.57 (s, 3H), 3.07 (s, 3H), 1.85˜1.93 (m, 6H), 1.72˜1.82 (m, 6H), 1.35 (s, 9H).

### Preparation of Compound 3

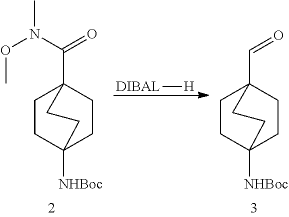

At −78° C., to a solution of 2 (960 mg, 3.1 mmol, 1.0 eq) in dried THF (30 mL) was added DIBAL-H (7.7 mL, 7.7 mmol, 2.5 eq) droppwise, and the solution was stirred until the starting material disappeared on TLC. Treated by saturated NH₄Cl solution and extracted by EtOAc, the organic layers were washed with brine, dried over sodium sulfate and concentrated. The residue was purified by flash-chromatography to give 3 (560 mg, 72.0%). ¹H-NMR (400 MHz, MeOH-d4) δ ppm 9.42 (s, 1H), 4.36 (s, 1H), 1.84˜1.92 (m, 6H), 1.66˜1.74 (m, 6H), 1.40 (s, 9H).

### Preparation of Compound 4

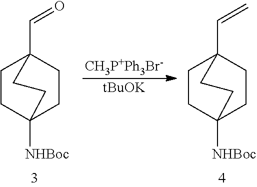 [SMILES: "tBuOK" ; CC(C)(C)OC(=O)NC12CCC(C=O)(CC1)CC2 ; C=CC12CCC(NC(=O)OC(C)(C)C)(CC1)CC2 ; "CH3P+Ph3Br-"]

At 0° C., to a suspension of CH₃P⁺Ph₃Br⁻ (1.79 g, 5.0 mmol, 2.5 eq) in dried THF (30 mL) was added tBuOK (560 mg, 5.0 mmol, 2.5 eq) portionwise under the protection of nitrogen. The mixture was stirred at the temperature for 1 h and a solution of 3 (506 mg, 2.0 mmol, 1.0 eq) in dried THF was added droppwise. Then the mixture was stirred at r.t. for 2 h before partitioned between water and EtOAc. The organic layers were washed with brine, dried over sodium sulfate and concentrated. The residue was purified by flash-chromatography to give 4 (412 mg, 82.1%). ¹H-NMR (400 MHz, CDCl₃) δ ppm 5.67˜5.74 (m, 1H), 4.80˜4.88 (m, 2H), 4.33 (s, 1H), 1.80˜1.86 (m, 6H), 1.54˜1.60 (m, 6H), 1.42 (s, 9H).

### Preparation of Compound 5

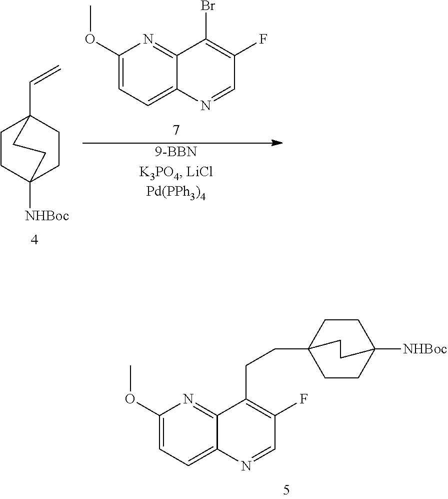

A solution of 4 (100 mg, 0.4 mmol, 1.0 eq) in dried THF (3 mL) was added 9-BBN (2 mL) at 0° C. under the protection of nitrogen, and then kept stirred at r.t. for 3 h, cooled to 0° C. and water (0.5 mL) was added. The mixture as stirred for another 1 h and 7 (103 mg, 0.4 mmol, 1.0 eq), K₃PO₄ (600 mg), LiCl (100 mg), Pd(PPh₃)₄ (100 mg) and EtOH (2 mL) was added. The resulting mixture was stirred at 70° C. under N₂ overnight before partitioned between water and EtOAc. The organic layers were washed with brine, dried over sodium sulfate and concentrated to give crude 5 (86 mg, crude, yield 50.3%), which was used for the next setp directly.

### Preparation of Compound 6

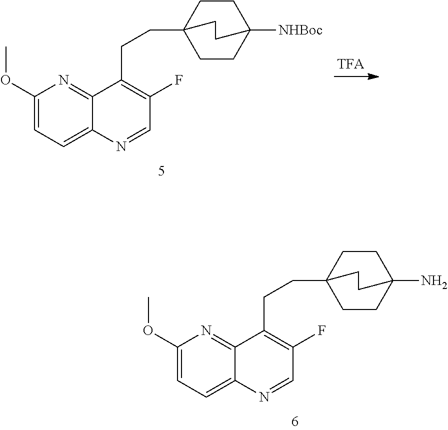 [SMILES: COc1ccc2ncc(F)c(CCC34CCC(NC(=O)OC(C)(C)C)(CC3)CC4)c2n1 ; COc1ccc2ncc(F)c(CCC34CCC(N)(CC3)CC4)c2n1]

A solution of 5 (86 mg, 0.2 mmol, 1.0 eq) in DCM (5 mL) was added TFA (5 mL), and the solution was stirred at r.t. for 1 h and concentrated. The residue was partitioned between saturated sodium carbonate solution and EtOAc. The organic layers were washed with brine, dried over sodium sulfate and concentrated to give 6 (41 mg, 62.1%), which was used for the next step directly. MS m/z 330 (M+1)⁺.

### Preparation of Example 196

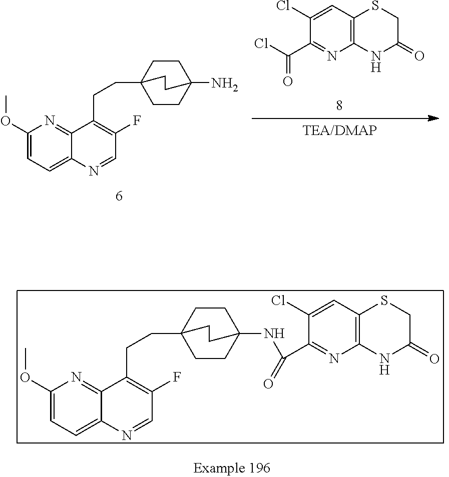 [SMILES: COc1ccc2ncc(F)c(CCC34CCC(N)(CC3)CC4)c2n1 ; O=C1CSc2cc(Cl)c(C(=O)Cl)nc2N1 ; COc1ccc2ncc(F)c(CCC34CCC(NC(=O)c5nc6c(cc5Cl)SCC(=O)N6)(CC3)CC4)c2n1]

At 0° C., to a suspension of 6 (45 mg, 0.15 mmol, 1.0 eq) and 8 (79 mg, 0.30 mmol, 2.0 eq) was added Et₃N (30 mg, 0.3 mmol, 2.0 eq) and then DMAP (40 mg, 0.3 mmol, 2.0 eq). The mixture was stirred at r.t. for 2 h, concentrated and dissolved into DMF. The solution was purified by prep-HPLC to give Example 196 (11 mg, 12.7%). ¹H-NMR (400 MHz, DMSO-d6) δ ppm 8.73 (s, 1H), 8.24˜8.26 (d, J=9.39 Hz, 1H), 8.06 (s, 1H), 8.01 (s, 1H), 7.20˜7.22 (d, J=9.39 Hz, 1H), 4.01 (s, 3H), 3.56 (s, 2H), 2.98˜3.06 (m, 2H), 1.86˜1.92 (m, 6H), 1.54˜1.60 (m, 6H), 1.34˜1.40 (m, 2H). MS m/z 556 (M+1)⁺.

### Example 197

### 1-(2-(3-Fluoro-6-methoxy-1,5-naphthyridin-4-yl)ethyl)-N-((7-fluoro-8-methyl-3-oxo-3,4-dihydro-2H-pyrido[3,2-b][1,4]oxazin-6-yl)methyl)-2-oxabicyclo[2.2.2]octan-4-aminium chloride

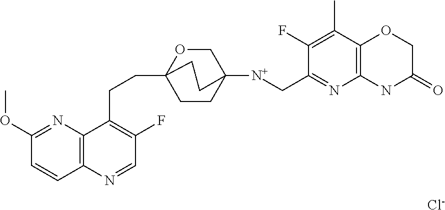 [SMILES: COc1ccc2ncc(F)c(CCC34CCC([NH2+]Cc5nc6c(c(C)c5F)OCC(=O)N6)(CC3)CO4)c2n1 ; [Cl-]]

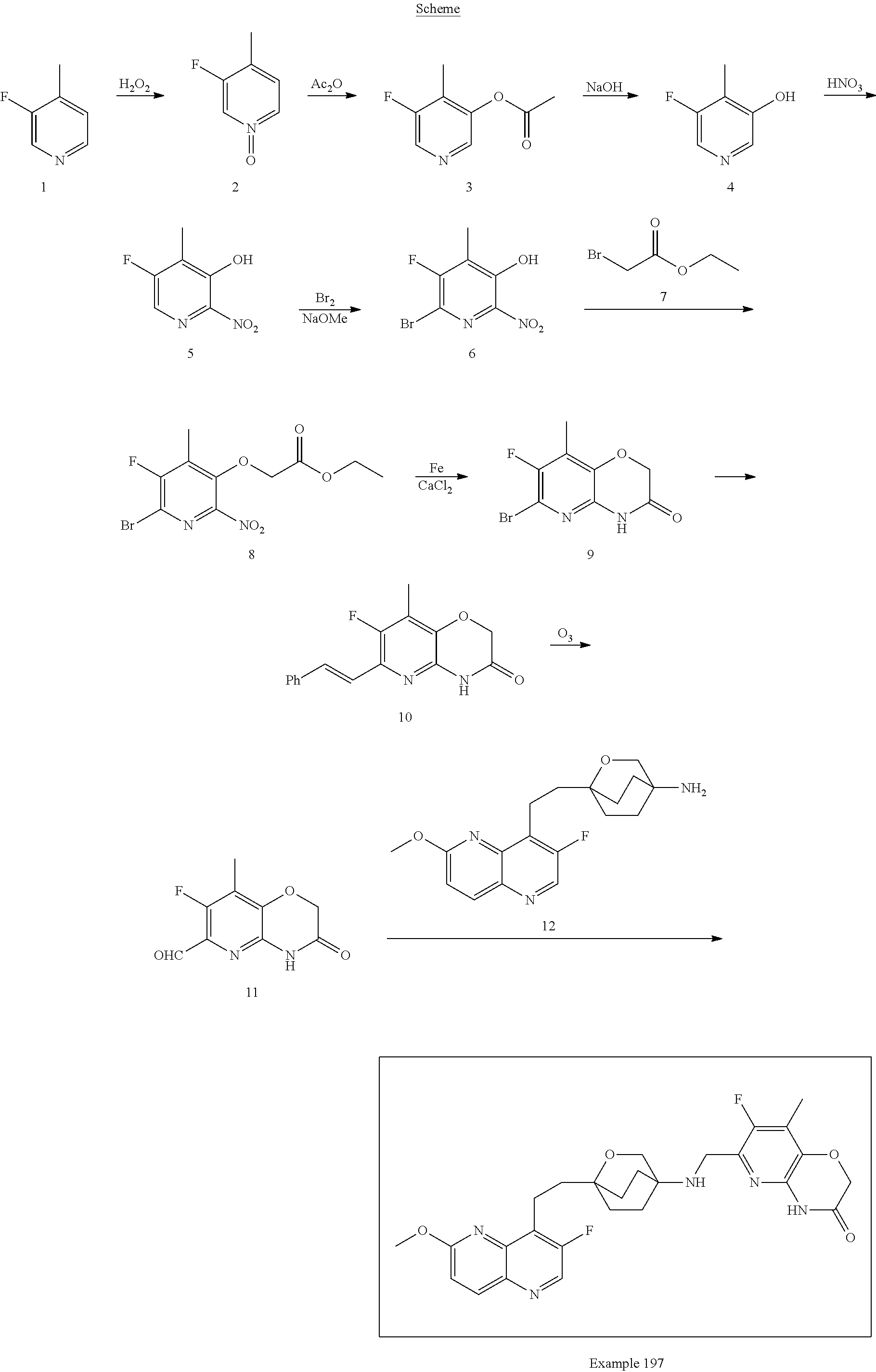

### Preparation of Compound 2

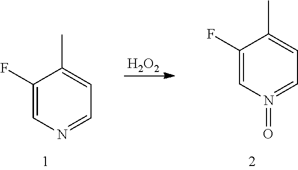

To a solution of 1 (40 g, 1.14 mol) in AcOH (280 mL) was added H₂O₂ (49 mL) and the mixture was heated under reflux for 20 hours. The reaction mixture was concentrated in vacuo and the resulting mixture solid 2 (40 g, 88.9%), which was used without purther purification.

### Preparation of Compound 3

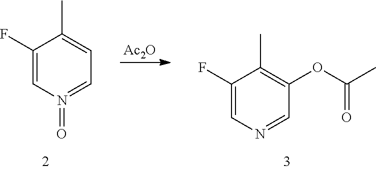

Acetic anhydride (300 mL) was heated under reflux and the oil bath was removed. Then 2 (40 g, 0.31 mol) was added in portions to maintain heating under reflux. After the addition was complete (0.5 hour), the reaction mixture was removed under reduced pressure and the residue obtained was stirred with a saturated solution of sodium bicarbonate (200 mL). The mixture was extracted with DCM. The organic layers were dried and concentrated to give crude 3.

### Preparation of Compound 4

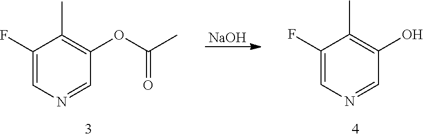

To a solution of 3 (40 g, 0.24 mol) in EtOH (300 mL) was added NaOH (13.2 g, 0.33 mol). The mixture was stirred at room temperature overnight. The reaction mixture was concentrated in vacuo to remove EtOH. The residue obtained was dissolved in water (100 mL) and then neutralized to pH 7 by the addition of concentrated hydrochloric acid. The neutral solution was extracted with EtOAc (3*100 mL). The organic layers were dried and concentrated to give crude 4 (25 g, 83.3%), which was used for next step without further purification.

### Preparation of Compound 5

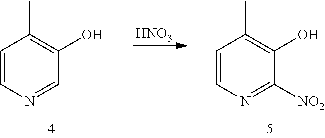

To 250 mL of conc.H₂SO₄ at 0° C. was added crude 4 (25 g, 0.196 mol) and then nitric acid (fuming, 10 mL) was added dropwise below 10° C., and the mixture was stirred at 10-20° C. for 2 hr and then poured to ice water. The mixture was adjusted to pH 2 by the addition of 8 N NaOH and extracted with EtOAc (2*200 mL). The extracts were combined, dried and concentrated. The residue was purified by column chromatography (PE:EtOAc=5:1) to give 5 (10 g, 29.5%). ¹H-NMR (400 MHz, DMSO-d₆) δ ppm 10.69 (s, 1H), 8.0 (s, 1H), 2.32 (s, 3H).

### Preparation of Compound 6

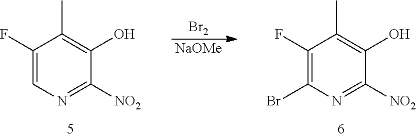

To a solution of 5 (1.5 g, 8.7 mmol) in MeOH (40 mL) was added 28% sodium methoxide in MeOH (9 mL). The mixture was stirred at room temperature for 30 min and then cooled with an ice bath. A solution of bromine (0.57 mL) in MeOH (1 mL) was added dropwise. The reaction mixture was stirred at 0° C. for 3 hours and concentrated to give residue. Then the residue was diluted with water, and the resulting precipitates were filtered off as product 6 (1.8 g, 81.8%). ¹H-NMR (400 MHz, CDCl₃) δ ppm 10.64 (s, 1H), 2.32 (s, 3H).

### Preparation of Compound 8

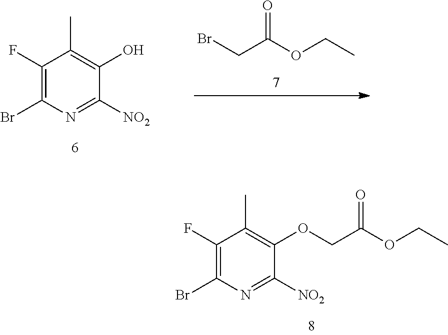

To a suspension of 6 (1.8 g, 7.2 mmol) and potassium carbonate (3 g, 21.7 mmol) in acetone (40 mL) was added ethyl bromoacetate (2.4 g, 14.3 mmol), and the mixture was heated at reflux for 8 hours. After dilution of the mixture with t-butyl methyl ether (60 mL), the resulting precipitates were filtered off. The filtrate was concentrated in vacuo to give 8 (2.6 g, 97%), which was used for the next step without further purification.

### Preparation of Compound 9

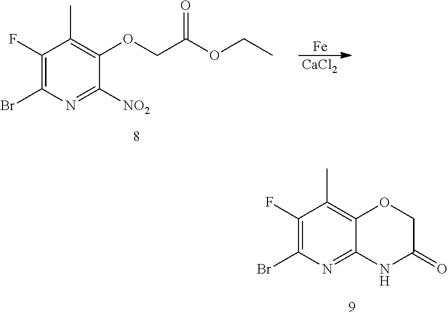

A suspension of the crude 8 (2.6 g, 7.72 mmol), iron powder (3.5 g, 62.5 mmol) and CaCl₂ (0.43 g, 3.9 mmol) in EtOH (100 mL) and water (20 mL) was heated under reflux for 5 hours. After dilution of the mixture EtOH (100 mL), the resulting precipitates were filtered off. The filtrate was concentrated in vacuo and the residue was purified via flash column chromatography (PE:EtOAc=5:1) to give 9 (1 g, 50%). ¹H-NMR (400 MHz, CDCl₃) δ ppm 4.57 (s, 2H), 2.23 (s, 3H).

### Preparation of Compound 10

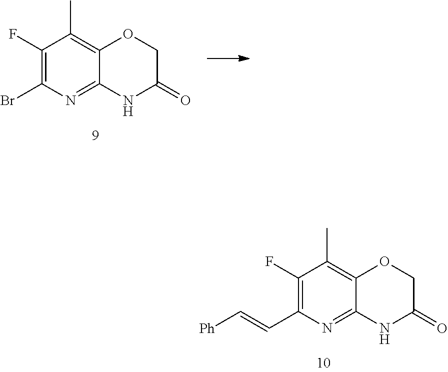 [SMILES: Cc1c(F)c(Br)nc2c1OCC(=O)N2 ; Cc1c(F)c(/C=C/c2ccccc2)nc2c1OCC(=O)N2]

To a degassed solution of 9 (1 g, 3.83 mmol) in 1,4-dioxane (60 mL) and water (10 mL) was added phenylvinylboronic acid (0.57 g, 3.85 mmol), potassium carbonate (1.06 g, 7.68 mmol) and tetrakis(triphenylphosphine)palladium (100 mg), and the mixture was heated at reflux for 24 hours. After diluted with water, the mixture was extracted with EtOAc. The organic layer was washed with brine, dried over anhydrous Na₂SO₄ and condensed. The residue was purified via flash column chromatography (silica gel, PE:EtOAc=10:1-3:1) to give 10 (0.4 g, 36.7%).

¹H-NMR (400 MHz, CDCl₃) δ ppm 7.91 (s, 1H), 7.56-7.21 (m, 6H), 4.68 (s, 2H), 2.22 (s, 3H).

MS m/z 285 (M+1)⁺.

### Preparation of Compound 11

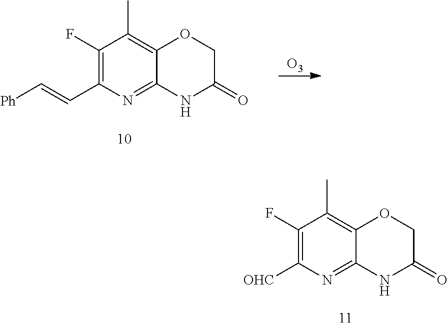 [SMILES: Cc1c(F)c(/C=C/c2ccccc2)nc2c1OCC(=O)N2 ; "O3" ; Cc1c(F)c(C=O)nc2c1OCC(=O)N2]

A suspension of 10 (0.4 g, 1.4 mmol) in dichloromethane (60 mL) and methanol (20 mL) was bubbled with ozone at 78° C. until a pale blue color appeared. The exess ozone was removed by bubbling air through the suspension for 30 min. Dimethylsulfide (1 mL) was added to the suspension. The mixture was stirred at room temperature for 30 min and concentrated in vacuo to give the cude product then purified by prep-TLC (PE:EA=1:1) to give 11 (0.2 g, 67.8%). ¹H-NMR (400 MHz, CDCl₃) δ ppm 10.5 (s, 1H), 8.4 (s, 1H), 4.8 (s, 2H), 2.23 (s, 3H).

### Preparation of Example 197

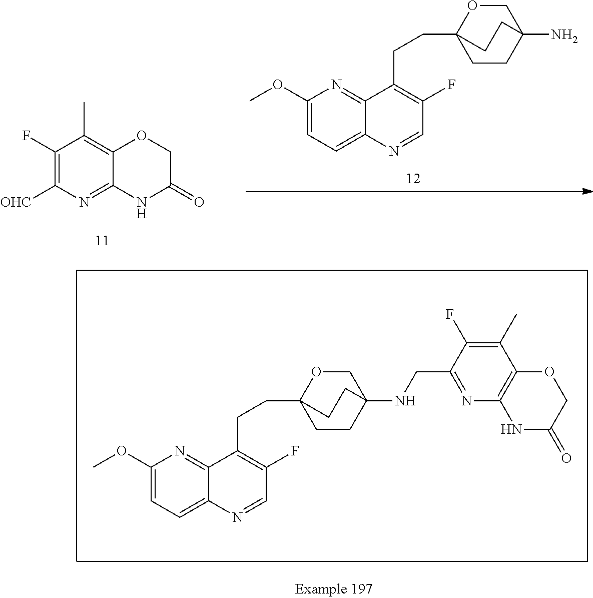 [SMILES: Cc1c(F)c(C=O)nc2c1OCC(=O)N2 ; COc1ccc2ncc(F)c(CCC34CCC(N)(CC3)CO4)c2n1 ; COc1ccc2ncc(F)c(CCC34CCC(NCc5nc6c(c(C)c5F)OCC(=O)N6)(CC3)CO4)c2n1]

Compound 12 (40 mg, 0.12 mmol) and 11 (40 mg, 0.19 mmol) in anhydrous DMF (3.5 mL) was added acetic acid (0.5 mL) was stirred at room temperature overnight. The resulting solution was cooled with ice-water bath and sodium triacetoxyborohydride (40 mg, 0.19 mmol) was added then stirred at room temperature for 1 hour. The residue was purified by prep-HPLC to afford Example 197. ¹H-NMR (400 MHz, MeOD) δ ppm 8.99 (d, J=4 Hz, 1H), 8.33 (d, J=8.0 Hz, 1H), 7.40 (d, J=8.0 Hz, 1H), 4.72 (s, 2H), 4.25 (s, 2H), 4.16 (s, 3H), 3.99 (s, 2H), 3.42-3.38 (t, J=8 Hz 2H), 2.22-2.10 (m, 9H), 2.00-1.97 (m, 2H), 1.89-1.85 (m, 2H). MS m/z 526 (M+1)⁺.

### Example 198

### N-((7-Ethyl-8-methyl-3-oxo-3,4-dihydro-2H-pyrido[3,2-b][1,4]oxazin-6-yl)methyl)-1-(2-(3-fluoro-6-methoxy-1,5-naphthyridin-4-yl)ethyl)-2-oxabicyclo[2.2.2]octan-4-aminium chloride

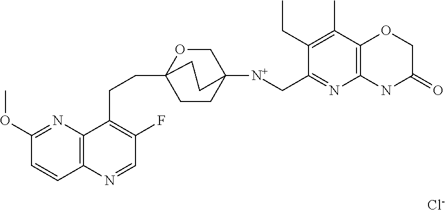 [SMILES: CCc1c(C[NH2+]C23CCC(CCc4c(F)cnc5ccc(OC)nc45)(CC2)OC3)nc2c(c1C)OCC(=O)N2 ; [Cl-]]

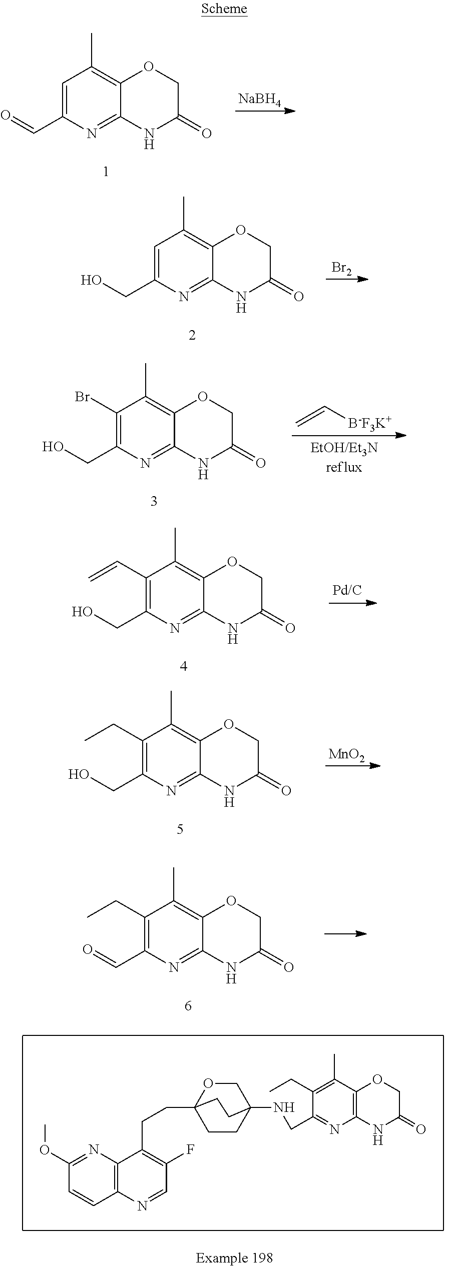

### Preparation of Compound 2

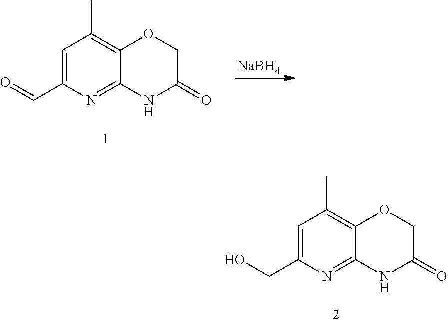

At 0° C., to a solution of 1 (3.5 g, 18.2 mmol, 1.0 eq) in MeOH (100 mL) was added NaBH₄ (2.1 g, 54.7 mmol, 3.0 eq) portionwise, and the mixture was stirred at the temperature for 1 h until 1 disappeared on TLC. The mixture was partitioned between water and EtOAc, and the organic layers were washed with brine, dried over sodium sulfate and concentrated. The residue was recrystallized from PE to give 2 (2.1 g, 60.0%), which was used for the next step directly.

### Preparation of Compound 3

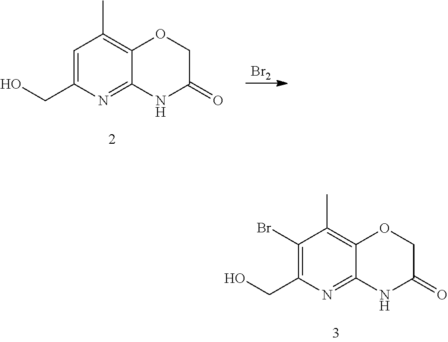

To a solution of 2 (2.1 g, 10.8 mmol, 1.0 eq) in DMF (20 mL) was added Br₂ (2.1 g, 13.0 mmol, 1.2 eq) dropwise at 0° C., then the mixture was stirred at r.t. for 3 h before treated by saturated sodium bicarbonate solution and extracted by EtOAc. The organic layers were washed with brine, dried over sodium sulfate and concentrated. The residue was recrystallized from PE to give 3 (1.9 g, 65.5%). MS m/z 273, 275 (M+1)⁺.

### Preparation of Compound 4

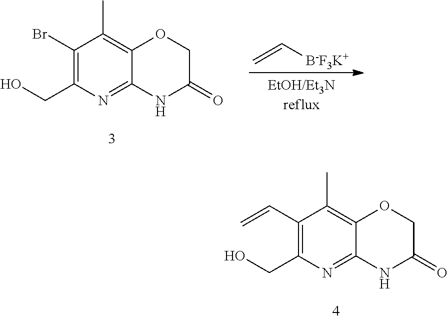

A suspension of 3 (816 mg, 3.0 mmol, 1.0 eq), potassium vinyltrifluoroborate (1.21 g, 9.0 mmol, 3.0 eq) and Pd(PPh₃)₂Cl₂ (100 mg, cat.) in EtOH (15 mL) and Et₃N (15 mL) was stirred under the protection of nitrogen at reflux for 5 h. The mixture was partitioned between water and EtOAc. The organic layers were washed with brine, dried over sodium sulfate and concentrated. The residue was purified by prep-TLC to give 4 (402 mg, 61.2%).

### Preparation of Compound 5

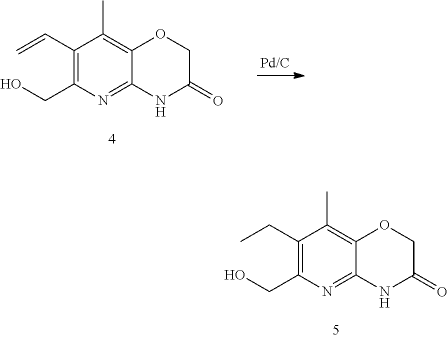 [SMILES: C=Cc1c(CO)nc2c(c1C)OCC(=O)N2 ; CCc1c(CO)nc2c(c1C)OCC(=O)N2]

To a solution of 4 (110 mg, 0.5 mmol, 1.0 eq) in MeOH (30 mL) was added PdC (100 mg, cat.) and the mixture was stirred at r.t. under H₂ for about 3 h until 4 disappeared on TLC. Then filtered and the filtrate was concentrated to give 5 (89 mg, 80.2%). MS m/z 223(M+1)⁺.

### Preparation of Compound 6

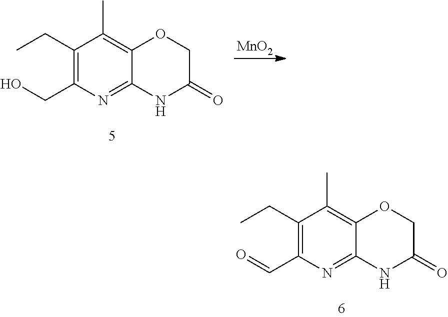

To a solution of 5 (89 mg, 0.4 mmol, 1.0 eq) in THF (15 mL) and DCM (15 mL) was added MnO₂ (348 mg, 4.0 mmol, 10.0 eq), and then the mixture was stirred under reflux overnight. Filtered and the filtrate was concentrated to give 6 (89 mg, 75.0%), which was used for next step directly.

### Preparation of Example 198

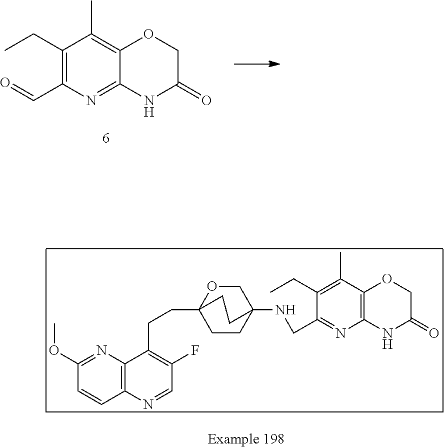 [SMILES: CCc1c(C=O)nc2c(c1C)OCC(=O)N2 ; CCc1c(CNC23CCC(CCc4c(F)cnc5ccc(OC)nc45)(CC2)OC3)nc2c(c1C)OCC(=O)N2]

A solution of 6 (66 mg, 0.3 mmol, 1.5 eq) and the Amine (66 mg, 0.2 mmol, 1.0 eq) in DMF:AcOH=7:1 (5 mL) was stirred at 30° C. for 15 h, and NaBH(OAc)₃ (127 mg, 0.6 mmol, 3.0 eq) was added. The mixture was stirred at r.t. for 2 h, and filtered. The filtrate was purified by prep-HPLC to give Example 198 (31 mg, 29.0%). ¹H-NMR (400 MHz, MeOD) δ ppm 9.00 (s, 1H), 8.33-8.35 (d, J=9.26 Hz, 1H), 7.41˜7.43 (d, J=9.26 Hz, 1H), 4.67 (s, 2H), 4.24 (s, 2H), 4.16 (s, 3H), 4.02 (s, 2H), 3.39˜3.43 (m, 2H), 2.64˜2.72 (m, 2H), 2.09˜2.23 (m, 6H), 1.86˜2.00 (m, 4H), 1.12˜1.16 (t, J=7.49 Hz, J=7.49 Hz, 3H). MS m/z 536 (M+1)⁺.

### Example 199

### 1-(2-(3-Fluoro-6-methoxy-1,5-naphthyridin-4-yl)ethyl)-N-((8-methyl-3-oxo-7-vinyl-3,4-dihydro-2H-pyrido[3,2-b][1,4]oxazin-6-yl)methyl)-2-oxabicyclo[2.2.2]octan-4-aminium chloride

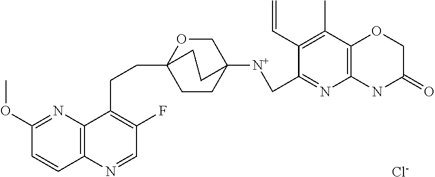 [SMILES: C=Cc1c(C[NH2+]C23CCC(CCc4c(F)cnc5ccc(OC)nc45)(CC2)OC3)nc2c(c1C)OCC(=O)N2 ; [Cl-]]

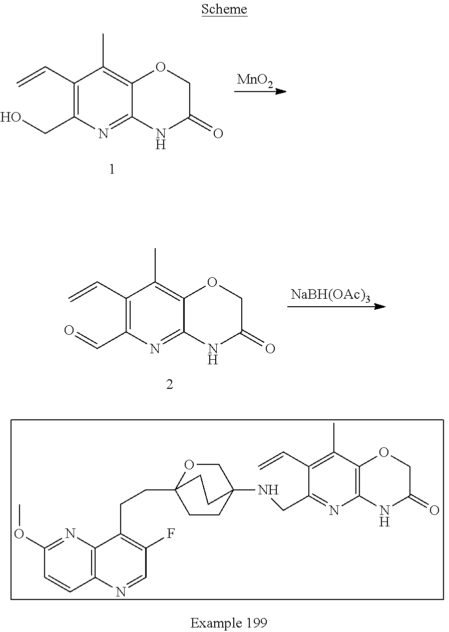

### Preparation of Compound 2

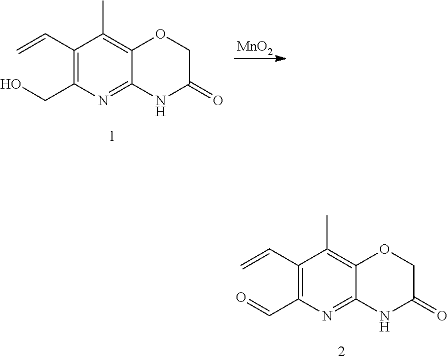

To a solution of 1 (110 mg, 0.5 mmol, 1.0 eq) in THF (15 mL) and DCM (15 mL) was added MnO₂ (435 mg, 5.0 mmol, 10.0 eq), and the mixture was stirred under reflux overnight. Filtered and the filtrate was concentrated to give 2 (83 mg, 76.1%), which was used for next step directly.

### Preparation of Example 199

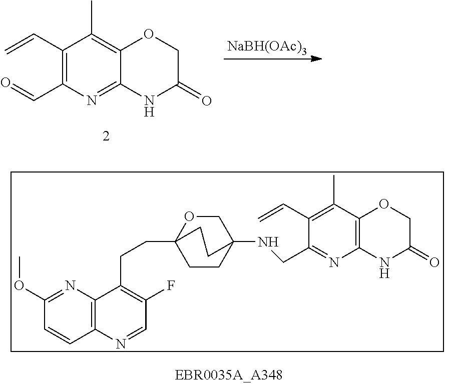 [SMILES: C=Cc1c(C=O)nc2c(c1C)OCC(=O)N2 ; C=Cc1c(CNC23CCC(CCc4c(F)cnc5ccc(OC)nc45)(CC2)OC3)nc2c(c1C)OCC(=O)N2 ; "NaBH(OAc)3"]

A solution of 2 (83 mg, 0.4 mmol, 2.0 eq) and the amine (66 mg, 0.2 mmol, 1.0 eq) in DMF:AcOH=7:1 (5 mL) was stirred at 30° C. for 15 h, and then NaBH(OAc)₃ (127 mg, 0.6 mmol, 3.0 eq) was added. The mixture was stirred at r.t. for 2 h, and then filtered. The filtration was purified by prep-HPLC to give Example 199 (29 mg, 51.1%). ¹H-NMR (MeOD, 400 MHz) δ ppm 8.94 (s, 1H), 8.30˜8.32 (d, J=8.82 Hz, 1H), 7.26˜7.28 (d, J=9.26 Hz, 1H), 6.69˜6.76 (m, 1H), 5.75˜5.78 (m, 1H), 5.39˜5.44 (m, 1H), 4.70 (s, 2H), 4.25 (s, 2H), 4.15 (s, 3H), 3.98 (s, 2H), 3.35˜3.39 (m, 2H), 2.23 (s, 3H), 2.07˜2.18 (m, 6H), 1.84˜1.98 (m, 4H). MS m/z 534 (M+1)⁺.

### Examples 200 and 201

### (R)—N-((2,3-Dihydro-[1,4]dioxino[2,3-c]pyridin-7-yl)methyl)-4-(2-(3-fluoro-6-methoxy-1,5-naphthyridin-4-yl)-1-hydroxyethyl)bicyclo[2.2.2]octan-1-aminium chloride and (S)—N-((2,3-Dihydro-[1,4]dioxino[2,3-c]pyridin-7-yl)methyl)-4-(2-(3-fluoro-6-methoxy-1,5-naphthyridin-4-yl)-1-hydroxyethyl)bicyclo[2.2.2]octan-1-aminium chloride

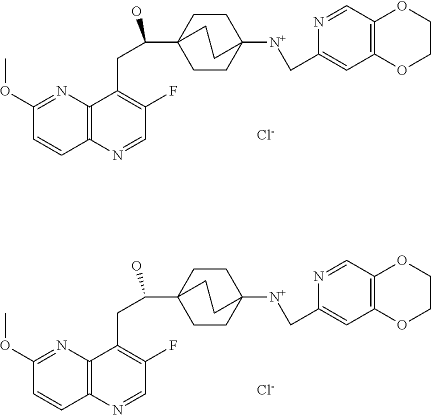 [SMILES: COc1ccc2ncc(F)c(C[C@@H](O)C34CCC([NH2+]Cc5cc6c(cn5)OCCO6)(CC3)CC4)c2n1 ; [Cl-] ; COc1ccc2ncc(F)c(C[C@H](O)C34CCC([NH2+]Cc5cc6c(cn5)OCCO6)(CC3)CC4)c2n1 ; [Cl-]]

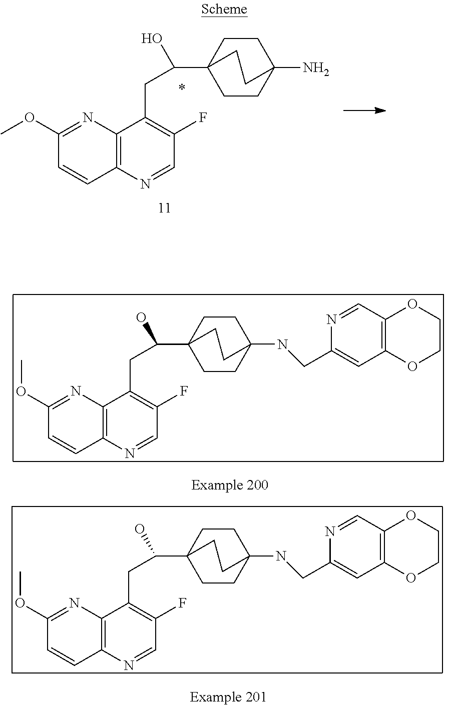

A solution of 11 (34.6 mg, single enantiomer, 0.1 mmol, 1.0 eq) in DMF:AcOH=7:1 (8 mL) was added aldehyde (26.4 mg, 2 mmol, 2.0 eq) and the mixture was stirred at 30° C. for 15 h. Then NaBH(OAc)₃ (49 mg, 2 mmol, 2.0 eq) was added, and then the mixture was stirred at r.t. for 2 h. Filtered, and the filtrate was purified by prep-HPLC to give the desired product.

Example 200 (from the faster eluted siomer, 20 mg, 40%) ¹H-NMR (MeOD, 400 MHz) δ ppm 8.91 (s, 1H), 8.46 (s, 1H), 8.31 (d, J=9.26 Hz, 1H), 7.58 (s, 1H), 7.35 (d, J=9.26 Hz, 1H), 4.58˜4.60 (m, 2H), 4.46˜4.48 (m, 2H), 4.39 (s, 2H), 4.13 (s, 3H), 3.78 (d, J=11.9 Hz, 1H), 3.53 (d, J=11.9 Hz, 1H), 3.21˜3.25 (m, 1H), 1.8˜2.02 (m, 12H). MS m/z 495 (M+1)⁺.

Example 201 (from the slower eluted siomer) ¹H-NMR (MeOD, 400 MHz) δ ppm 8.91 (s, 1H), 8.42 (s, 1H), 8.31 (d, J=9.26 Hz, 1H), 7.45 (s, 1H), 7.35 (d, J=9.26 Hz, 1H), 4.55-4.57 (m, 2H), 4.44-4.46 (m, 2H), 4.39 (s, 2H), 4.13 (s, 3H), 3.78 (d, J=11.9 Hz, 1H), 3.53 (d, J=11.9 Hz, 1H), 3.21-3.25 (m, 1H), 1.8-2.02 (m, 12H). MS m/z 495 (M+1)⁺.

### Example 202

### 1-(2-(6-Cyano-3-oxo-2H-benzo[b][1,4]oxazin-4(3H)-yl)ethyl)-N-((3-oxo-3,4-dihydro-2H-pyrido[3,2-b][1,4]oxazin-6-yl)methyl)-2-oxabicyclo[2.2.2]octan-4-aminium chloride

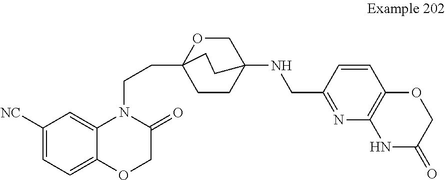 [SMILES: N#Cc1ccc2c(c1)N(CCC13CCC(NCc4ccc5c(n4)NC(=O)CO5)(CC1)CO3)C(=O)CO2]

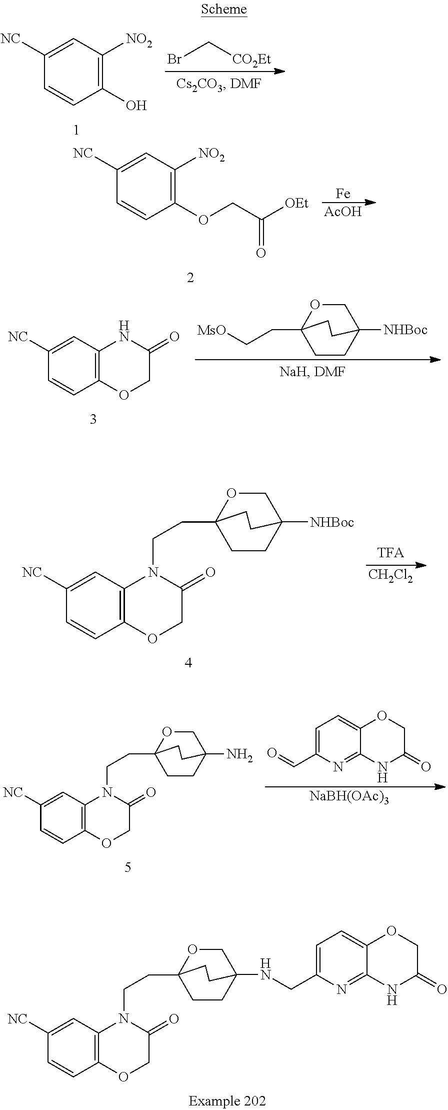

### Preparation of Compound 2

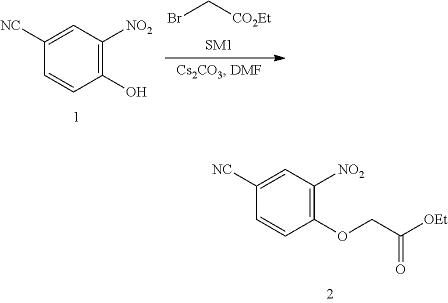 [SMILES: CCOC(=O)CBr ; N#Cc1ccc(O)c([N+](=O)[O-])c1 ; CCOC(=O)COc1ccc(C#N)cc1[N+](=O)[O-]]

To a mixture of 1 (0.32 g, 2 mmol) and Cs₂CO₃ (1.3 g, 4 mmol) in DMF (10 mL) was added SM1 (0.5 g, 3 mmol). The mixture was stirred for 3 h at room temperature. Then the mixture was poured into water and extracted with EtOAc. The combined organic phases were washed with brine, dried over Na₂SO₄, filtered and concentrated. The residue was purified by column chromatography on silica gel (PE:EtOAc=10:1) to give the product of 2 (0.3 g, yield: 60%).

¹H NMR (400 MHz CDCl₃) δ 8.16 (s, 1H), 7.80 (d, J=8.8 Hz, 1H), 7.05 (d, J=8.8 Hz, 1H), 4.84 (s, 2H), 4.25 (q, J=7.2 Hz, 2H), 1.28 (t, J=7.2 Hz, 3H).

### Preparation of Compound 3

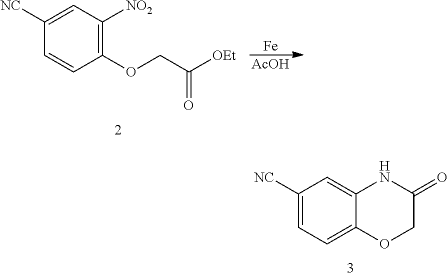

A mixture of 2 (300 mg, 1.2 mmol), ferrous powder (390 mg, 6 mmol) in AcOH (10 mL) was refluxed for 4 h. The mixture was filtered and the filtrate was concentrated. The residue was purified by prep-TLC (PE:EtOAc=2:1) to give the product of 3 (100 mg, yield: 48%).

¹H NMR (400 MHz CD₃OD) δ 7.32 (d, J=8.4 Hz, 1H), 7.18 (s, 1H), 7.06 (d, J=8.4 Hz, 1H), 4.68 (s, 2H).

### Preparation of Compound 4

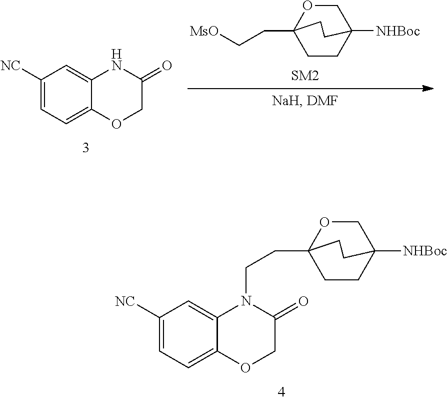 [SMILES: CC(C)(C)OC(=O)NC12CCC(CCOS(C)(=O)=O)(CC1)OC2 ; N#Cc1ccc2c(c1)NC(=O)CO2 ; CC(C)(C)OC(=O)NC12CCC(CCN3C(=O)COc4ccc(C#N)cc43)(CC1)OC2]

A mixture of 3 (50 mg, 0.28 mmol), SM2 (100 mg, 0.28 mmol), NaH (20 mg, 0.84 mmol) in DMF (3 mL) was stirred for 12 h at 90° C. The reaction was quenched with water and extracted with EtOAc. The combined organic phases were washed with brine, dried over Na₂SO₄, filtered and concentrated. The residue was purified by prep-TLC (PE:EtOAc=2:1) to give the product of 4 (30 mg, yield: 25%).

¹H NMR (400 MHz CDCl₃) δ 7.34 (s, 1H), 7.24 (d, J=8.8 Hz, 1H), 6.96 (d, J=8.8 Hz, 1H), 4.59 (s, 2H), 3.89˜3.95 (m, 4H), 1.80˜2.02 (m, 6H), 1.60˜1.66 (m, 4H), 1.36 (s, 9H).

### Preparation of Compound 5

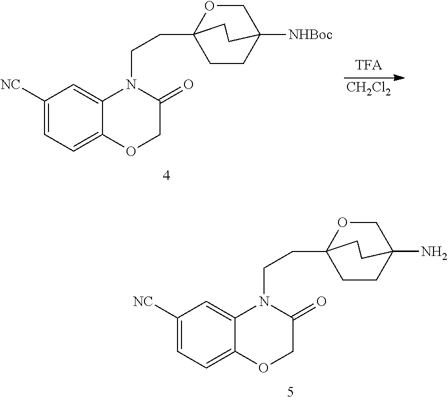 [SMILES: CC(C)(C)OC(=O)NC12CCC(CCN3C(=O)COc4ccc(C#N)cc43)(CC1)OC2 ; "CH2Cl2" ; N#Cc1ccc2c(c1)N(CCC13CCC(N)(CC1)CO3)C(=O)CO2]

A mixture of 4 (100 mg, 0.23 mmol) in DCM/TFA (3 mL1 mL) was stirred at room temperature for 1 h. Then the mixture was concentrated to give the crude product of 5. The crude product was used in the next step directly.

### Preparation of Example 202

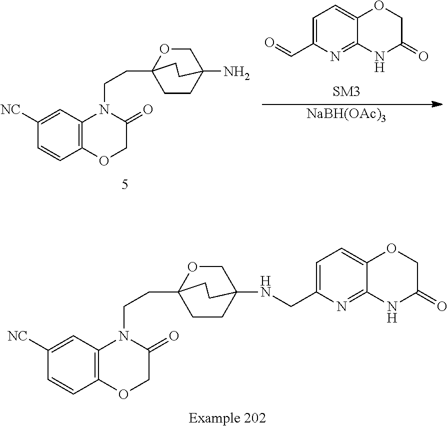 [SMILES: N#Cc1ccc2c(c1)N(CCC13CCC(N)(CC1)CO3)C(=O)CO2 ; O=Cc1ccc2c(n1)NC(=O)CO2 ; N#Cc1ccc2c(c1)N(CCC13CCC(NCc4ccc5c(n4)NC(=O)CO5)(CC1)CO3)C(=O)CO2 ; "NaBH(OAc)3"]

A mixture of 5 (75 mg, 0.23 mmol), SM3 (41 mg, 0.23 mmol), AcOH (0.1 mL) in DMF (2 mL) was stirred at room temperature overnight. Then NaBH(OAc)₃ (147 mg, 0.69 mmol) was added into the mixture. The resulting mixture was stirred at room temperature for another 1 h. Then the mixture was basified to pH 8˜9 with aq. NaHCO₃ and extracted with EtOAc. The combined organic layers were washed with brine, dried over Na₂SO₄, filtered and concentrated. The residue was purified by prep-HPLC to give the product of Example 202 (20 mg, yield: 18%).

¹H NMR (400 MHz CD₃OD) δ 7.50 (s, 1H), 7.34˜7.40 (m, 2H), 7.07˜7.11 (m, 2H), 4.69 (s, 2H), 4.68 (s, 2H), 4.20 (s, 2H), 4.04˜4.07 (m, 2H), 4.02 (s, 2H), 2.05˜2.11 (m, 6H), 1.86˜1.91 (m, 2H), 1.73˜1.76 (m, 2H). MS m/z 490 (M+1)⁺.

### Example 203

### 1-(2-(6-Bromo-3-oxo-2H-benzo[b][1,4]oxazin-4(3H)-yl)ethyl)-N-((3-oxo-3,4-dihydro-2H-pyrido[3,2-b][1,4]oxazin-6-yl)methyl)-2-oxabicyclo[2.2.2]octan-4-aminium chloride

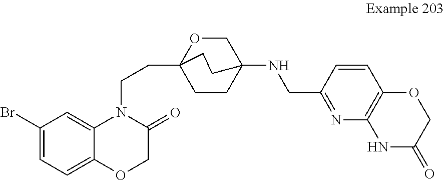 [SMILES: O=C1COc2ccc(CNC34CCC(CCN5C(=O)COc6ccc(Br)cc65)(CC3)OC4)nc2N1]

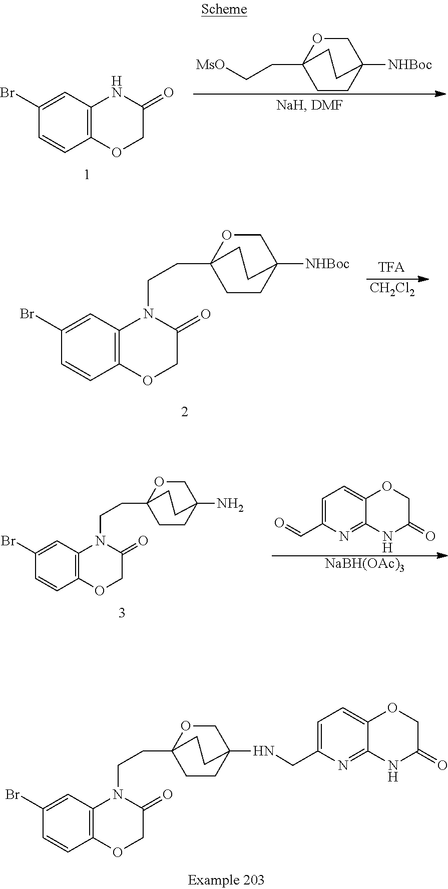

### Preparation of Compound 2

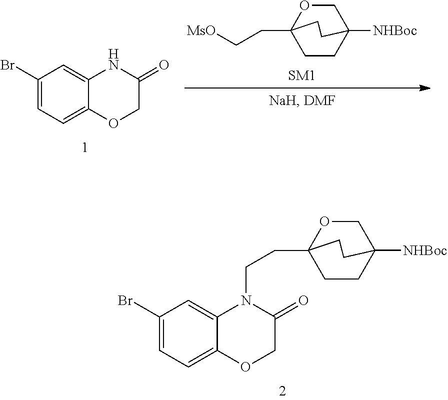 [SMILES: CC(C)(C)OC(=O)NC12CCC(CCOS(C)(=O)=O)(CC1)OC2 ; O=C1COc2ccc(Br)cc2N1 ; CC(C)(C)OC(=O)NC12CCC(CCN3C(=O)COc4ccc(Br)cc43)(CC1)OC2]

A mixture of 1 (65 mg, 0.28 mmol), SM1 (100 mg, 0.28 mmol), NaH (20 mg, 0.84 mmol) in DMF (3 mL) was stirred for 12 h at 90° C. The reaction was quenched with water and extracted with EtOAc. The combined organic phases were washed with brine, dried over Na₂SO₄, filtered and concentrated. The residue was purified by prep-TLC (PE:EtOAc=2:1) to give the product of 2 (40 mg, yield: 30%).

¹H NMR (400 MHz CDCl₃) δ 7.24 (s, 1H), 7.04 (d, J=8.4 Hz, 1H), 6.82 (d, J=8.4 Hz, 1H), 4.58 (s, 2H), 3.91˜3.95 (m, 4H), 1.80˜2.02 (m, 6H), 1.65˜1.70 (m, 4H), 1.40 (s, 9H).

### Preparation of Compound 3

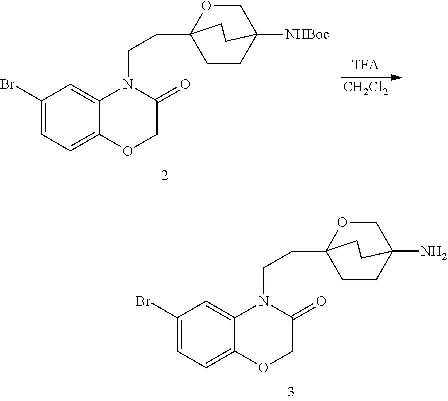 [SMILES: CC(C)(C)OC(=O)NC12CCC(CCN3C(=O)COc4ccc(Br)cc43)(CC1)OC2 ; "CH2Cl2" ; NC12CCC(CCN3C(=O)COc4ccc(Br)cc43)(CC1)OC2]

A mixture of 2 (100 mg, 0.2 mmol) in DCM/TFA (3 mL1 mL) was stirred at room temperature for 1 h. Then the mixture was concentrated to give the crude product of 3. The crude product was used in the next step directly.

### Preparation of Example 203

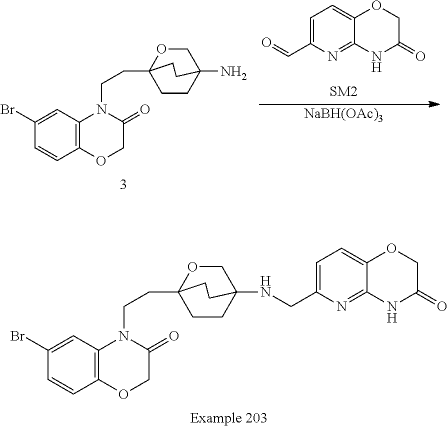 [SMILES: NC12CCC(CCN3C(=O)COc4ccc(Br)cc43)(CC1)OC2 ; O=Cc1ccc2c(n1)NC(=O)CO2 ; O=C1COc2ccc(CNC34CCC(CCN5C(=O)COc6ccc(Br)cc65)(CC3)OC4)nc2N1 ; "NaBH(OAc)3"]

A mixture of 3 (80 mg, 0.2 mmol), SM2 (38 mg, 0.2 mmol), AcOH (0.1 mL) in DMF (2 mL) was stirred at room temperature overnight. Then NaBH(OAc)₃ (127 mg, 0.6 mmol) was added into the mixture. The resulting mixture was stirred at room temperature for another 1 h. Then the mixture was basified to pH 8˜9 with aq. NaHCO₃ and extracted with EtOAc. The combined organic layers were washed with brine, dried over Na₂SO₄, filtered and concentrated. The residue was purified by prep-HPLC to give the product of Example 203 (30 mg, yield: 28%).

¹H NMR (400 MHz CD₃OD) δ 7.36 (d, J=8.0 Hz, 1H), 7.30 (s, 1H), 7.07˜7.14 (m, 2H), 6.90 (d, J=8.8 Hz, 1H), 4.68 (s, 2H), 4.57 (s, 2H), 4.20 (s, 2H), 3.99˜4.03 (m, 4H), 2.04˜2.10 (m, 6H), 1.86˜1.90 (m, 2H), 1.71˜1.75 (m, 2H). MS m/z 543 (M+1)⁺.

### Example 204

### (S)-1-(2-(3-Fluoro-6-methoxy-1,5-naphthyridin-4-yl)-1-hydroxyethyl)-N-((3-oxo-3,4-dihydro-2H-benzo[b][1,4]thiazin-6-yl)methyl)-2-oxabicyclo[2.2.2]octan-4-aminium chloride

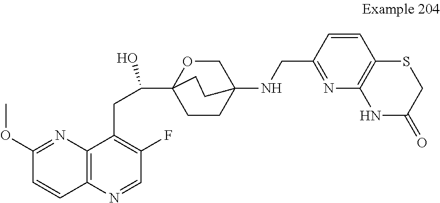 [SMILES: COc1ccc2ncc(F)c(C[C@H](O)C34CCC(NCc5ccc6c(n5)NC(=O)CS6)(CC3)CO4)c2n1]

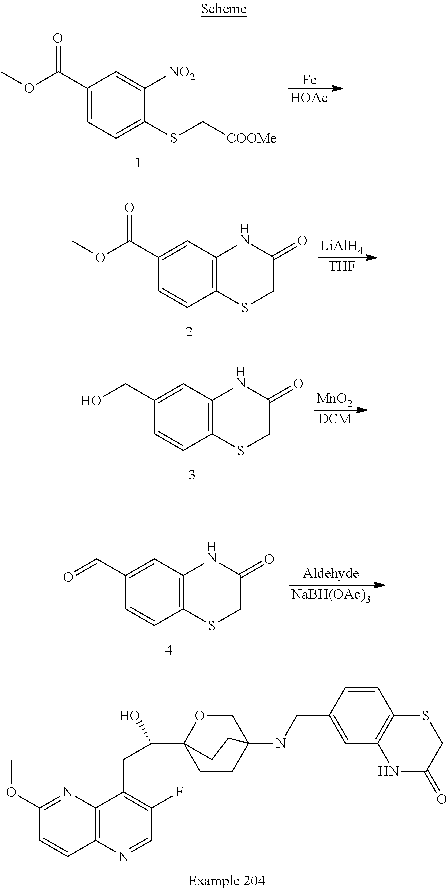

### Preparation of Compound 2

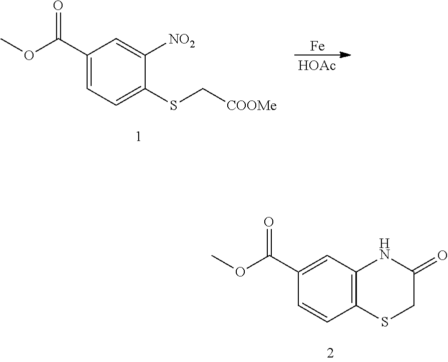

A mixture of 1 (800 mg, 2.8 mmol), ferrous powder (780 mg, 14 mmol) in AcOH (10 mL) was stirred at 80° C. for 1 h. The mixture was filtered, washed with EtOAc and the filtrate was concentrated. The residue was purified by column chromatography on silica gel (PE:EtOAc=5:1) to give the product of 2 (450 mg, yield: 72%).

¹H NMR (400 MHz CDCl₃) δ 8.86 (br, 1H), 7.71 (d, J=8.0 Hz, 1H), 7.61 (s, 1H), 7.40 (d, J=8.0 Hz, 1H), 3.96 (s, 3H), 3.51 (s, 2H).

### Preparation of Compound 3

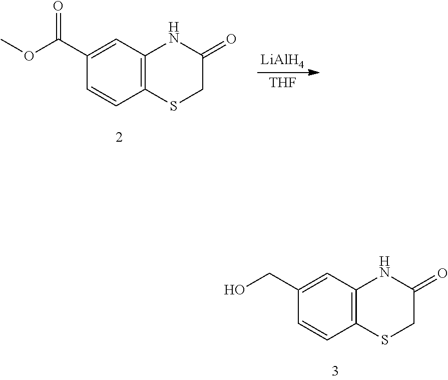

To a mixture of 2 (450 mg, 2 mmol) in THF (10 mL) was added LiAlH₄ (76 mg, 2 mmol) at 0° C. The resulting mixture was stirred at 0° C. for 1 h. Then the reaction was quenched with water (0.1 mL), dried over Na₂SO₄, filtered and concentrated. The residue was purified by column chromatography on silica gel (DCM:MeOH=20:1) to give the product of 3 (200 mg, yield: 51%).

¹H NMR (400 MHz DMSO d₆) δ 9.57 (br, 1H), 7.12 (d, J=8.4 Hz, 1H), 6.84˜6.86 (m, 2H), 4.46 (s, 2H), 3.93 (br, 1H), 3.23 (s, 2H).

### Preparation of Compound 4

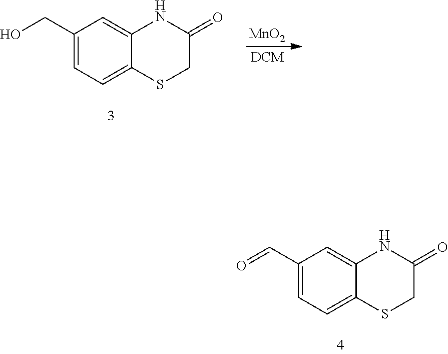

A mixture of 3 (60 mg, 0.3 mmol) and MnO₂ (78 mg, 0.9 mmol) in DCM (3 mL) was stirred for 4 h at 60° C. The mixture was filtered and the filtrate was concentrated to give the product of 4 (20 mg, yield: 33%).

¹H NMR (400 MHz CDCl₃) δ 9.86 (s, 1H), 7.46 (s, 1H), 7.40˜7.42 (m, 2H), 3.41 (s, 2H).

### Preparation of Example 204

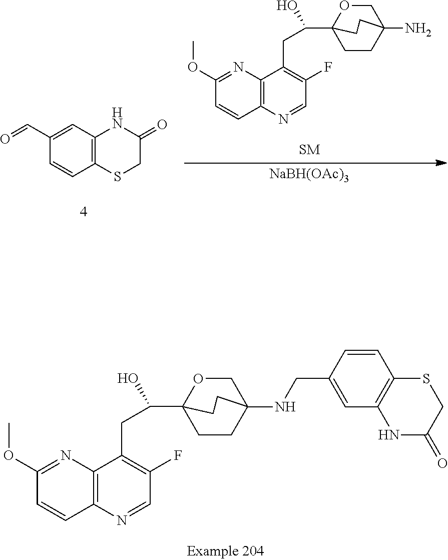

A mixture of 4 (50 mg, 0.14 mmol), SM (28 mg, 0.14 mmol), AcOH (0.1 mL) in DMF (2 mL) was stirred at room temperature overnight. Then NaBH(OAc)₃ (90 mg, 0.42 mmol) was added into the mixture. The resulting mixture was stirred at room temperature for another 1 h.

Then the mixture was basified to pH 8˜9 with aq. NaHCO₃ and extracted with EtOAc. The combined organic layers were washed with brine, dried over Na₂SO₄, filtered and concentrated. The residue was purified by prep-HPLC to give the product of Example 204 (15 mg, yield: 20%).

¹H NMR (400 MHz CD₃OD) δ 8.61 (s, 1H), 8.29 (d, J=8.0 Hz, 1H), 7.66 (d, J=8.0 Hz, 1H), 7.10˜7.16 (m, 2H), 7.03 (s, 1H), 4.21 (s, 2H), 4.07˜4.13 (m, 3H), 3.96˜3.99 (m, 3H), 3.49˜3.53 (m, 1H), 3.43 (s, 2H), 3.19˜3.22 (m, 1H), 1.97˜2.30 (m, 8H). MS m/z 525 (M+1)⁺.

### Example 205

### (S)—N-((1,1-Dioxido-3-oxo-3,4-dihydro-2H-benzo[b][1,4]thiazin-6-yl)methyl)-1-(2-(3-fluoro-6-methoxy-1,5-naphthyridin-4-yl)-1-hydroxyethyl)-2-oxabicyclo[2.2.2]octan-4-aminium chloride

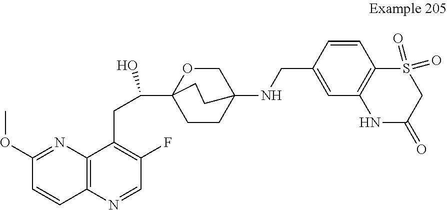 [SMILES: COc1ccc2ncc(F)c(C[C@H](O)C34CCC(NCc5ccc6c(c5)NC(=O)CS6(=O)=O)(CC3)CO4)c2n1]

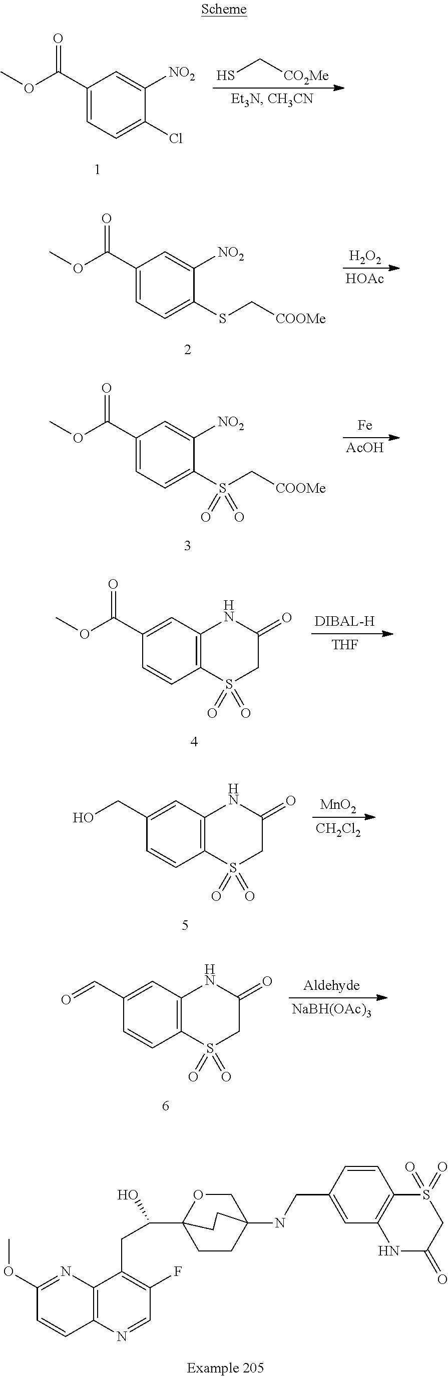

### Preparation of Compound 2

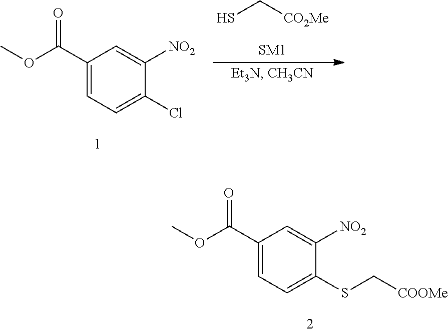

A mixture of 1 (5.0 g, 23 mmol), SM1 (3.6 g, 35 mmol) and NEt₃ (0.5 mL) in MeCN (100 mL) was stirred for 5 h at 80° C. The mixture was concentrated and the residue was purified by column chromatography on silica gel (PE:EtOAc=10:1) to give the product of 2 (3.0 g, yield: 46%).

¹H NMR (400 MHz CDCl₃) δ 8.85 (s, 1H), 8.16 (d, J=8.0 Hz, 1H), 7.54 (d, J=8.0 Hz, 1H), 3.96 (s, 3H), 3.79 (s, 2H), 3.75 (s, 3H).

### Preparation of Compound 3

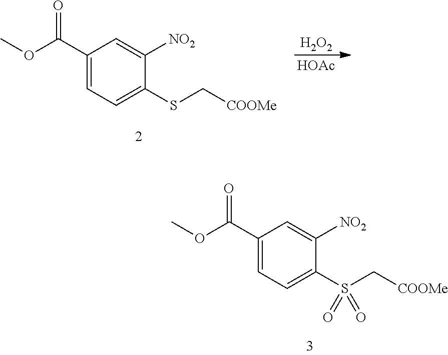 [SMILES: COC(=O)CSc1ccc(C(=O)OC)cc1[N+](=O)[O-] ; "H2O2" ; COC(=O)CS(=O)(=O)c1ccc(C(=O)OC)cc1[N+](=O)[O-] ; "HOAc"]

H₂O₂ (2 mL) was added into a stirred solution of 2 (500 mg, 1.7 mmol) in AcOH (10 mL). The mixture was stirred at 60° C. for 2 h and then cooled to room temperature. Water was added. The formed precipitate was filtered off, washed with water, and dried to give the product of 3 (250 mg, yield: 45%).

¹H NMR (400 MHz CDCl₃) δ 8.52 (s, 1H), 8.46 (d, J=8.0 Hz, 1H), 8.32 (d, J=8.0 Hz, 1H), 4.72 (s, 2H), 4.05 (s, 3H), 3.78 (s, 3H).

### Preparation of Compound 4

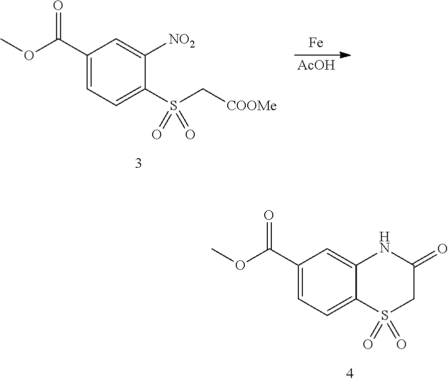

A mixture of 3 (600 mg, 1.9 mmol), ferrous powder (630 mg, 9.5 mmol) in AcOH (10 mL) was stirred for 2 h at 90° C. The mixture was filtered and washed with EtOAc. The filtrate was concentrated. The residue was purified by column chromatography on silica gel (PE:EtOAc=5:1) to give the product of 4 (350 mg, yield: 72%).

¹H NMR (400 MHz CDCl₃) δ 8.10 (d, J=8.0 Hz, 1H), 7.90 (d, J=8.0 Hz, 1H), 7.84 (s, 1H), 4.37 (s, 2H), 4.09 (s, 3H).

### Preparation of Compound 5

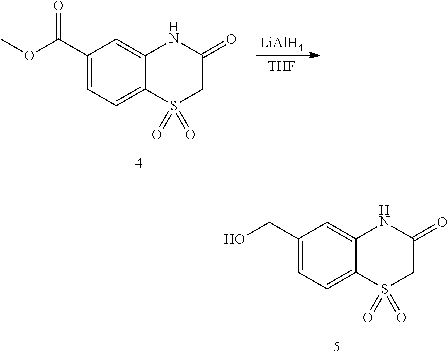 [SMILES: COC(=O)c1ccc2c(c1)NC(=O)CS2(=O)=O ; "LiAlH4" ; "THF" ; O=C1CS(=O)(=O)c2ccc(CO)cc2N1]

To a mixture of 4 (350 mg, 1.37 mmol) in THF (10 mL) was added LiAlH₄ (52 mg, 1.37 mmol) at 0° C. The resulting mixture was stirred at 0° C. for 2 h. Then the reaction was quenched with water (0.1 mL), dried over Na₂SO₄, filtered and concentrated to give the product of 5 (180 mg, yield: 58%).

¹H NMR (400 MHz CDCl₃) δ 7.73 (d, J=8.4 Hz, 1H), 7.14˜7.16 (m, 2H), 4.60 (s, 2H), 4.10 (s, 2H).

### Preparation of Compound 6

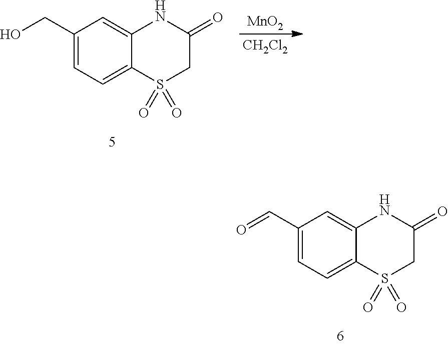

A mixture of 5 (150 mg, 0.66 mmol) and MnO₂ (170 mg, 1.98 mmol) in DCM (5 mL) was stirred for 4 h at 60° C. The mixture was filtered and the filtrate was concentrated. The residue was purified by prep-TLC (DCM:MeOH=20:1) to give 6 (30 mg, yield: 20%).

### Preparation of Example 205

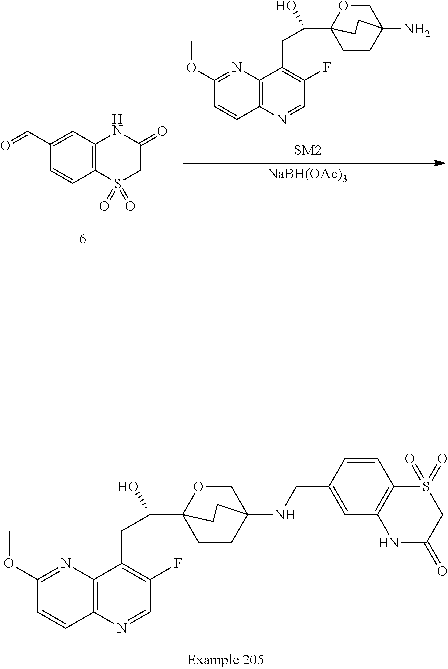 [SMILES: COc1ccc2ncc(F)c(C[C@H](O)C34CCC(N)(CC3)CO4)c2n1 ; O=Cc1ccc2c(c1)NC(=O)CS2(=O)=O ; "SM2" ; COc1ccc2ncc(F)c(C[C@H](O)C34CCC(NCc5ccc6c(c5)NC(=O)CS6(=O)=O)(CC3)CO4)c2n1 ; "NaBH(OAc)3"]

A mixture of 6 (46 mg, 0.13 mmol), SM2 (30 mg, 0.13 mmol), AcOH (0.1 mL) in DMF (2 mL) was stirred at room temperature overnight. Then NaBH(OAc)₃ (82 mg, 0.39 mmol) was added into the mixture. The resulting mixture was stirred at room temperature for another 1 h. Then the mixture was basified to pH 8˜9 with aq. NaHCO₃ and extracted with EtOAc. The combined organic layers were washed with brine, dried over Na₂SO₄, filtered and concentrated. The residue was purified by prep-HPLC to give the product of Example 205 (15 mg, yield: 20%).

¹H NMR 1 (400 MHz CD₃OD) δ 8.61 (s, 1H), 8.19 (d, J=9.2 Hz, 1H), 7.95 (d, J=8.0 Hz, 1H), 7.43 (d, J=8.0 Hz, 1H), 7.30 (s, 1H), 7.16 (d, J=9.2 Hz, 1H), 4.26 (s, 2H), 4.08 (s, 3H), 3.96˜4.01 (m, 3H), 3.49˜3.53 (m, 1H), 3.25 (s, 2H), 3.19˜3.22 (m, 1H), 1.98˜2.30 (m, 8H). MS m/z 557 (M+1)⁺.

### Example 206

### (S)-6-(((1-(2-(3-Fluoro-6-methoxy-1,5-naphthyridin-4-yl)-1-hydroxyethyl)-2-oxabicyclo[2.2.2]octan-4-yl)ammonio)methyl)-3,4-dihydro-2H-pyrido[3,2-b][1,4]oxazin-4-ium chloride

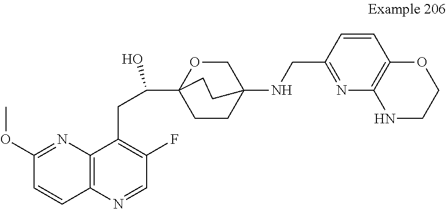 [SMILES: COc1ccc2ncc(F)c(C[C@H](O)C34CCC(NCc5ccc6c(n5)NCCO6)(CC3)CO4)c2n1]

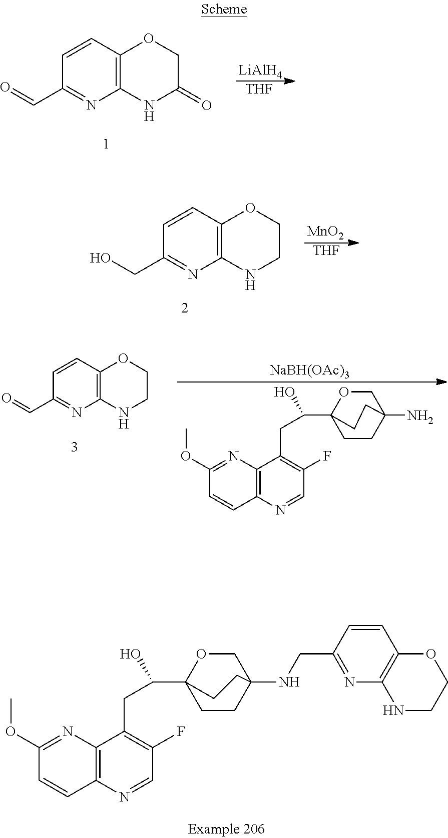

### Preparation of Compound 2

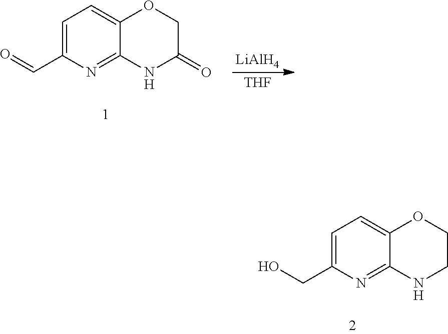 [SMILES: O=Cc1ccc2c(n1)NC(=O)CO2 ; "LiAlH4" ; OCc1ccc2c(n1)NCCO2 ; "THF"]

To a mixture of 1 (300 mg, 1.68 mmol) in THF (10 mL) was added LiAlH₄ (127 mg, 3.36 mmol) at 0° C. The resulting mixture was stirred at 0° C. for 2 h. Then the reaction was quenched with water (0.2 mL), dried over Na₂SO₄, filtered and concentrated to give the crude product of 2. The crude product was used in the next step directly.

### Preparation of Compound 3

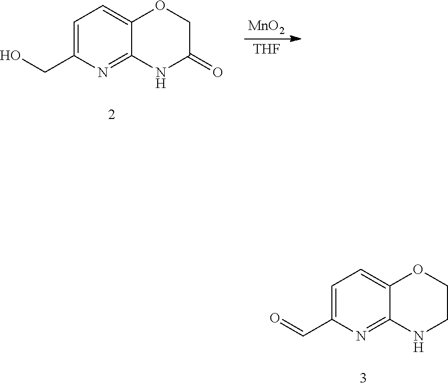

A mixture of 2 (250 mg, 1.5 mmol) and MnO₂ (345 mg, 6 mmol) in THF (10 mL) was stirred overnight at room temperature. The mixture was filtered and the filtrate was concentrated. The residue was purified by prep-TLC (DCM:MeOH=20:1) to give 3 (80 mg, yield: 30%).

### Preparation of Example 206

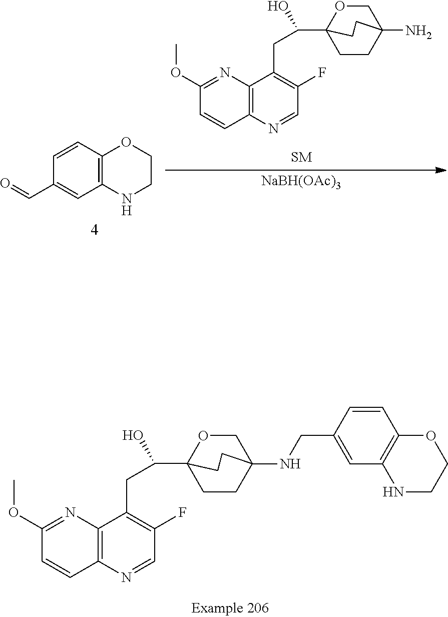

A mixture of 3 (70 mg, 0.42 mmol), SM (80 mg, 0.23 mmol), AcOH (0.1 mL) in DMF (2 mL) was stirred at room temperature overnight. Then NaBH(OAc)₃ (146 mg, 0.69 mmol) was added into the mixture. The resulting mixture was stirred at room temperature for another 1 h. Then the mixture was basified to pH 8˜9 with aq. NaHCO₃ and extracted with EtOAc. The combined organic layers were washed with brine, dried over Na₂SO₄, filtered and concentrated. The residue was purified by prep-HPLC to give the product of Example 206 (30 mg, yield: 26%).

¹H NMR (400 MHz CD₃OD) δ 8.59 (s, 1H), 8.18 (d, J=8.8 Hz, 1H), 7.15 (d, J=8.8 Hz, 1H), 6.90 (d, J=8.0 Hz, 1H), 6.52 (d, J=8.0 Hz, 1H), 4.11˜4.14 (m, 2H), 4.07 (s, 3H), 3.89˜3.91 (m, 1H), 3.74 (s, 2H), 3.57 (s, 2H), 3.46˜3.48 (m, 3H), 3.17˜3.12 (m, 1H), 2.03˜2.15 (m, 2H), 1.78˜1.92 (m, 6H). MS m/z 496 (M+1)⁺.

### Example 207

### 6-(((1-(2-(3-Fluoro-6-methoxy-1,5-naphthyridin-4-yl)ethyl)-2-oxabicyclo[2.2.2]octan-4-yl)ammonio)methyl)-2,3-dihydro-1H-pyrido[2,3-b][1,4]oxazin-1-ium chloride

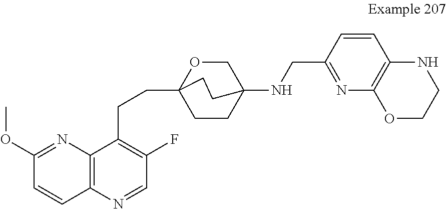 [SMILES: COc1ccc2ncc(F)c(CCC34CCC(NCc5ccc6c(n5)OCCN6)(CC3)CO4)c2n1]

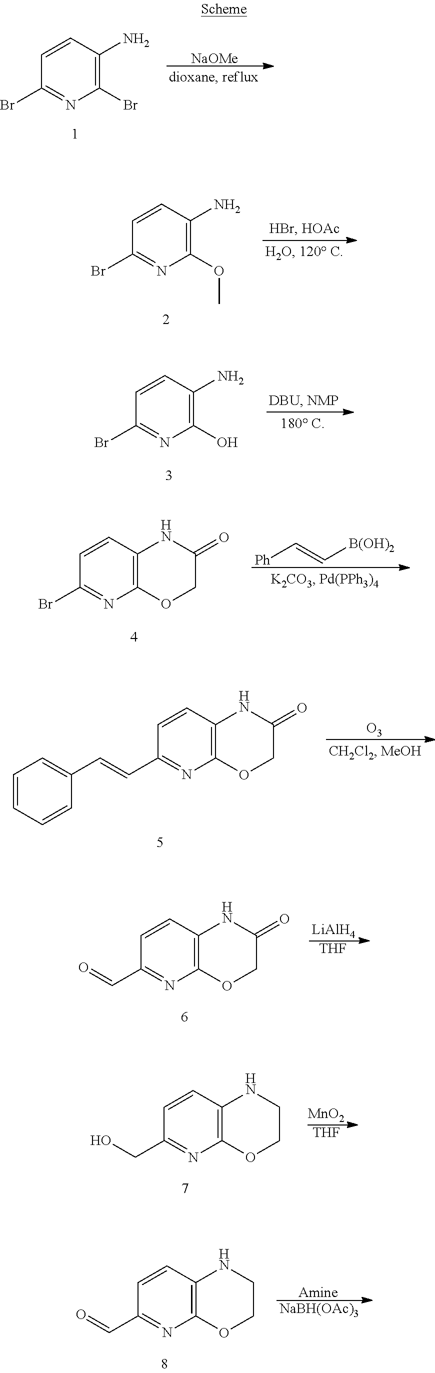

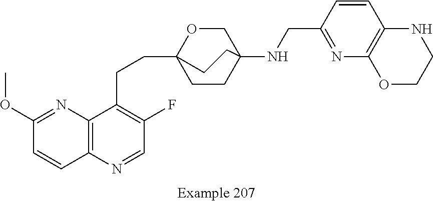 [SMILES: COc1ccc2ncc(F)c(CCC34CCC(NCc5ccc6c(n5)OCCN6)(CC3)CO4)c2n1]

### Preparation of Compound 2

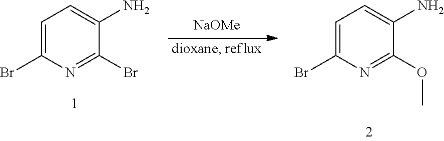

A mixture of 1 (5.2 g, 20.7 mmol) and NaOMe (7.8 g, 145 mmol) in dioxane (80 mL) was refluxed for 24 h. Then the reaction was quenched with water and extracted with EtOAc.

The combined organic layers were washed with brine, dried over Na₂SO₄, filtered and concentrated. The residue was purified by column chromatography on silica gel (PE:EtOAc=5:1) to give the product of 2 (3 g, yield: 72%).

¹H NMR (400 MHz CDCl₃) δ 6.84 (d, J=7.6 Hz, 1H), 6.74 (d, J=7.6 Hz, 1H), 3.95 (s, 3H).

### Preparation of Compound 3

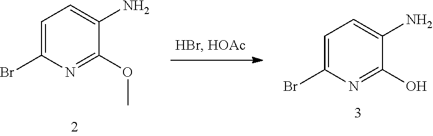

A mixture of 2 (2.0 g, 10 mmol) in 30% HBr/AcOH (20 mL) and water (10 mL) was refluxed for 1 h. Then the reaction mixture was concentrated. The residue was purified by column chromatography on silica gel (PE:EtOAc=4:1) to give the product of 3 (1.2 g, yield: 67%).

¹H NMR (400 MHz CD₃OD) δ 6.60 (d, J=7.6 Hz, 1H), 6.36 (d, J=7.6 Hz, 1H).

### Preparation of Compound 4

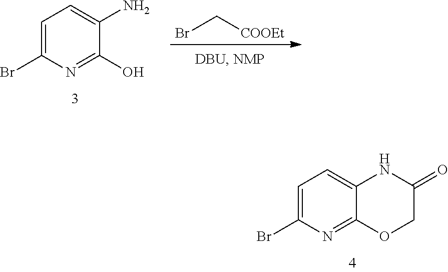 [SMILES: Nc1ccc(Br)nc1O ; CCOC(=O)CBr ; O=C1COc2nc(Br)ccc2N1]

To a mixture of 3 (1.0 g, 5.3 mmol) and BrCH₂COOEt (0.87 mL, 9.2 mmol) in NMP (20 mL) was added DBU (1.5 mL, 10 mmol). The resulting mixture was stirred at 150° C. for 5 h. Then the reaction was quenched with water and extracted with EtOAc. The combined organic layers were washed with brine, dried over Na₂SO₄, filtered and concentrated. The residue was purified by column chromatography on silica gel (PE:EtOAc=5:1) to give the product of 4 (0.6 g, yield: 60%).

¹H NMR (400 MHz DMSO d₆) δ 10.9 (br, 1H), 7.20 (d, J=7.6 Hz, 1H), 7.13 (d, J=7.6 Hz, 1H), 4.78 (s, 2H).

### Preparation of Compound 5

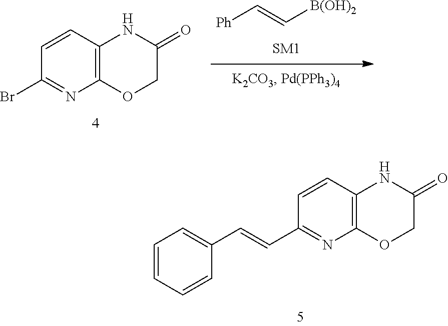

A mixture of 4 (400 mg, 1.7 mmol), styrylboronic acid (SM1, 265 mg, 1.7 mmol), K₂CO₃ (490 mg, 3.5 mmol) and Pd(PPh₃)₄ (60 mg, 0.05 mmol) in dioxaneH₂O (8 mL2 mL) was stirred overnight at 90° C. The mixture was filtered and the filltrate was diluted with water and extracted with EtOAc. The organic layers were washed with brine, dried over Na₂SO₄, filtered and concentrated. The residue was purified by column chromatography on silic gel (PE:EtOAc=10:1) to give the product of 5 (220 mg, yield: 50%).

¹H NMR (400 MHz DMSO d₆) δ 10.9 (br, 1H), 7.50˜7.61 (m, 5H), 7.37˜7.41 (m, 2H), 7.20 (d, J=7.6 Hz, 1H), 7.13 (d, J=7.6 Hz, 1H), 4.78 (s, 2H).

### Preparation of Compound 6

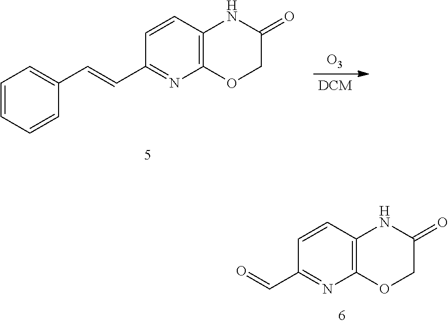

To a solution of 5 (150 mg, 0.6 mmol) in DCM (10 mL) and MeOH (5 mL) was bubbled with O₃ at −78° C. until a pale blue color appeared. The exess O₃ was removed by bubbling N₂ for 5 min. Then Me₂S (2 mL) was added to the mixture which was stirred overnight at room temperature. The mixture was concentrated. Hexane (5 mL) was added to the resulting residue which was stirred for 30 min and filtered. The solid was washed with hexane and dried to give 6 (100 mg, yield: 95%).

¹H NMR (400 MHz DMSO d₆) δ 9.69 (s, 1H), 7.61 (d, J=7.6 Hz, 1H), 7.36 (d, J=7.6 Hz, 1H), 4.85 (s, 2H).

### Preparation of Compound 7

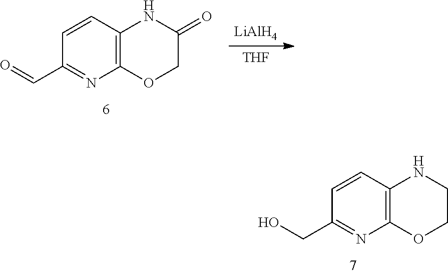

To a mixture of 6 (300 mg, 1.7 mmol) in THF (8 mL) was added LiAlH₄ (130 mg, 3.4 mmol) at 0° C. The resulting mixture was stirred at 0° C. for 2 h. Then the reaction was quenched with water (0.1 mL), dried over Na₂SO₄, filtered and concentrated to give the crude product of 7. The crude product was used in the next step without further purification.

### Preparation of Compound 8

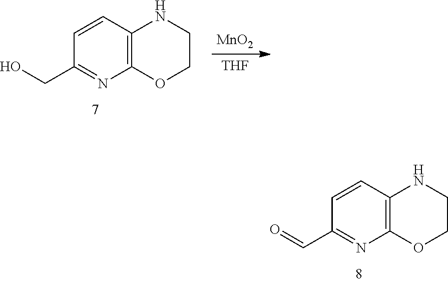

A mixture of 7 (280 mg, 1.68 mmol) and MnO₂ (500 mg, 62 mmol) in THF (10 mL) was stirred overnight at room temperature. The mixture was filtered and the filtrate was concentrated. The residue was purified by prep-TLC (DCM:MeOH=20:1) to give 8 (20 mg, yield: 11%).

¹H NMR (400 MHz CDCl₃) δ 9.69 (s, 1H), 7.49 (d, J=8.0 Hz, 1H), 6.83 (d, J=8.0 Hz, 1H), 4.37˜4.39 (m, 2H), 3.42˜3.47 (m, 2H).

### Preparation of Example 207

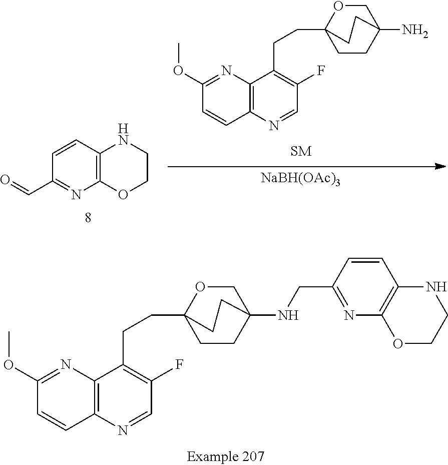 [SMILES: COc1ccc2ncc(F)c(CCC34CCC(N)(CC3)CO4)c2n1 ; O=Cc1ccc2c(n1)OCCN2 ; COc1ccc2ncc(F)c(CCC34CCC(NCc5ccc6c(n5)OCCN6)(CC3)CO4)c2n1 ; "NaBH(OAc)3"]

A mixture of 8 (20 mg, 0.12 mmol), SM (50 mg, 0.15 mmol), AcOH (0.4 mL) in DMF (2 mL) was stirred at room temperature overnight. Then NaBH(OAc)₃ (13.68 mg, 0.36 mmol) was added into the mixture. The resulting mixture was stirred at room temperature for another 1 h. Then the mixture was basified to pH 8˜9 with aq. NaHCO₃ and extracted with EtOAc. The combined organic layers were washed with brine, dried over Na₂SO₄, filtered and concentrated. The residue was purified by prep-HPLC to give the product of Example 207 (20 mg, yield: 35%).

¹H NMR (400 MHz CD₃OD) δ 8.59 (s, 1H), 8.19 (d, J=8.8 Hz, 1H), 7.16 (d, J=8.8 Hz, 1H), 6.96 (d, J=8.0 Hz, 1H), 6.89 (d, J=8.0 Hz, 1H), 4.34˜4.36 (m, 2H), 4.07 (s, 3H), 4.01 (s, 2H), 3.94 (m, 2H), 3.35˜3.37 (m, 2H), 3.21˜3.23 (m, 2H), 2.00˜2.13 (m, 6H), 1.90˜1.96 (m, 2H), 1.77˜1.81 (m, 2H). MS m/z 480 (M+1)⁺.

### Example 208

### 6-(((1-(2-(3-Fluoro-6-methoxy-1,5-naphthyridin-4-yl)-2-hydroxyethyl)-2-thiabicyclo[2.2.2]octan-4-yl)amino)methyl)-2H-pyrido[3,2-b][1,4]oxazin-3(4H)-one

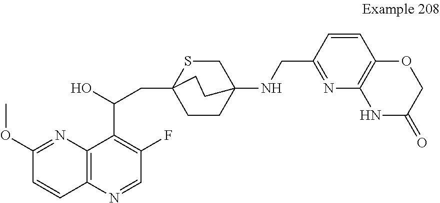 [SMILES: COc1ccc2ncc(F)c(C(O)CC34CCC(NCc5ccc6c(n5)NC(=O)CO6)(CC3)CS4)c2n1]

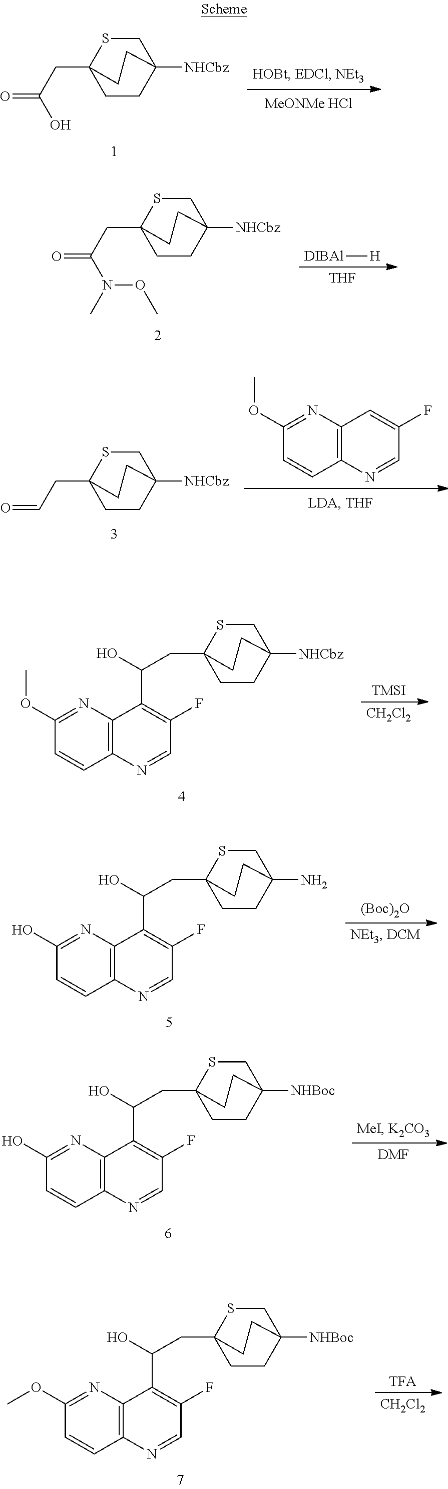

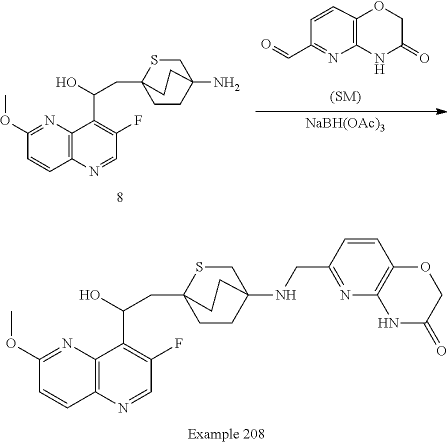 [SMILES: O=Cc1ccc2c(n1)NC(=O)CO2 ; COc1ccc2ncc(F)c(C(O)CC34CCC(N)(CC3)CS4)c2n1 ; COc1ccc2ncc(F)c(C(O)CC34CCC(NCc5ccc6c(n5)NC(=O)CO6)(CC3)CS4)c2n1 ; "NaBH(OAc)3"]

### Preparation of Compound 2

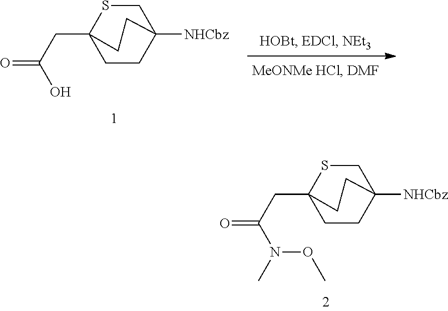 [SMILES: O=C(O)CC12CCC(NC(=O)OCc3ccccc3)(CC1)CS2 ; CON(C)C(=O)CC12CCC(NC(=O)OCc3ccccc3)(CC1)CS2]

To a solution of 1 (200 mg, 0.6 mmol) in DMF (8 mL) was added EDCI (176 mg, 0.9 mmol) and HOBT (124 mg, 0.9 mmol). The reaction mixture was stirred for 2 h at room temperature. Then N,O-dimethyl-hydroxylamine hydrochloride (70 mg, 0.72 mmol) and Et₃N (30 mg, 1.8 mmol) were added and the resulting mixture was stirred at room temperature for another 12 h. The reaction was quenched with water, extracted with EtOAc. The organic layer was washed with brine, dried over Na₂SO₄, filtered and the filtrate was concentrated in vacuo. The product (120 mg, yield: 73%) was purified by prep-TLC.

¹H NMR (400 MHz CDCl₃) δ 7.33˜7.25 (m, 5H), 5.00 (s, 2H), 3.64 (s, 3H), 3.12 (s, 3H), 3.05 (s, 2H), 2.52 (s, 2H), 2.23˜2.15 (m, 6H), 1.84˜1.76 (m, 2H).

### Preparation of Compound 3

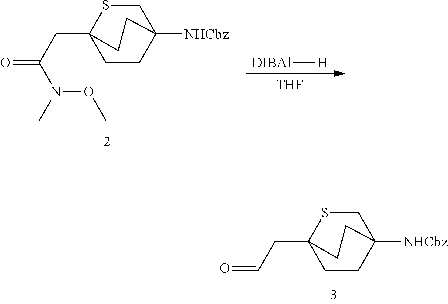

To a solution of 2 (200 mg, 0.53 mmol) in THF was added DIBAL-H (1.59 mL, 1.59 mmol) dropwise at −78° C. under N₂, then the mixture was stirred at −78° C. for 2 h. The reaction was quenched with water. Then Na₂SO₄ was added and the mixture was stirred for 30 min. The solid was removed by filtration. The filtrate was concentrated in vacuo. The product (100 mg, yield: 88%) was purified by prep-TLC.

### Preparation of Compound 4

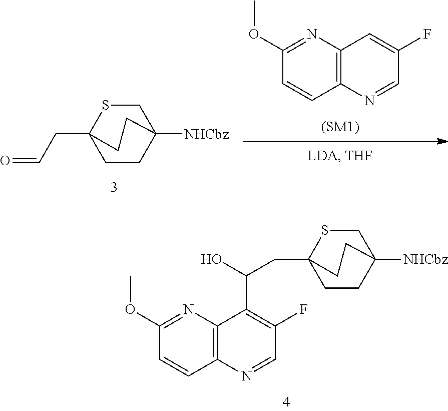 [SMILES: COc1ccc2ncc(F)cc2n1 ; O=CCC12CCC(NC(=O)OCc3ccccc3)(CC1)CS2 ; COc1ccc2ncc(F)c(C(O)CC34CCC(NC(=O)OCc5ccccc5)(CC3)CS4)c2n1]

To a solution of SM1 (178 mg, 1 mmol) in THF (8 mL) was added LDA (2.5 mL, 1 mmol) dropwise at −78° C. under N₂, then the mixture was stirred at −78° C. for 2 h. 3 (161 mg, 0.5 mmol) in THF (2 mL) was added and the mixture was stirred at −78° C. for another 3 h. The reaction was quenched with water, extracted with EtOAc. The organic layer was washed with brine, dried over Na₂SO₄, filtered and the filtrate was concentrated in vacuo. The product (100 mg, yield: 40%) was purified by prep-TLC.

¹H NMR (400 MHz CDCl₃) δ 8.60 (s, 1H), 8.20 (d, J=8.8 Hz, 1H), 7.34˜7.25 (m, 5H), 7.08 (d, J=8.8 Hz, 1H), 5.94˜5.91 (m, 1H), 5.63˜5.59 (m, 1H), 5.02 (s, 2H), 4.68˜4.65 (m, 1H), 4.04 (s, 3H), 3.10 (s, 2H), 2.39˜2.21 (m, 6H), 1.82˜1.78 (m, 2H).

### Preparation of Compound 5

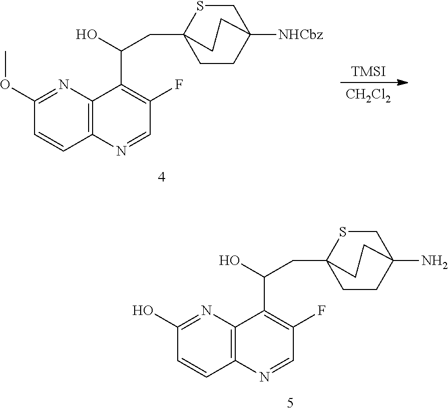 [SMILES: "TMSI" ; COc1ccc2ncc(F)c(C(O)CC34CCC(NC(=O)OCc5ccccc5)(CC3)CS4)c2n1 ; "CH2Cl2" ; NC12CCC(CC(O)c3c(F)cnc4ccc(O)nc34)(CC1)SC2]

TMSI (40 mg, 0.2 mmol) was added dropwise into a mixture of 4 (50 mg, 0.1 mmol) in DCM (3 mL) at 0° C. under N₂. The mixture was stirred at room temperature for 12 h. Then the mixture was concentrated to give 5. The crude compound was used in next step directly.

### Preparation of Compound 6

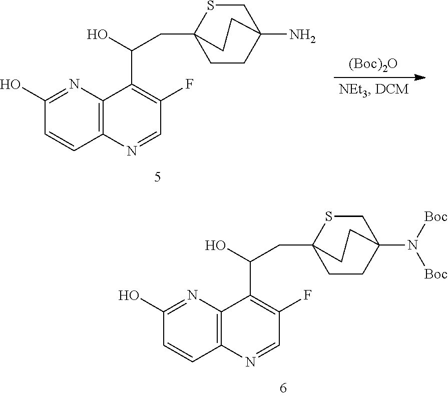

A mixture of 5 (40 mg, 0.11 mmol), (Boc)₂O (71 mg, 0.33 mmol), Et₃N (44 mg, 0.44 mmol) and DCM (2 mL) was stirred at room temperature for 12 h. Then the mixture was poured into water and extracted with DCM. The combined organic phases were washed with brine, dried over Na₂SO₄, filtered and the filtrate was concentrated. The crude compound was purified by pre-TLC (PE:EtOAc=2:1) (30 mg, yield: 50%).

¹H NMR (400 MHz CDCl₃) δ 8.75 (s, 1H), 8.43 (d, J=8.8 Hz, 1H), 7.34 (d, J=8.8 Hz, 1H), 5.61˜5.56 (m, 1H), 5.29˜5.26 (m, 1H), 4.42˜4.40 (m, 1H), 3.06 (s, 2H), 2.40˜2.12 (m, 8H), 1.74 (s, 9H), 1.63 (s, 9H).

### Preparation of Compound 7

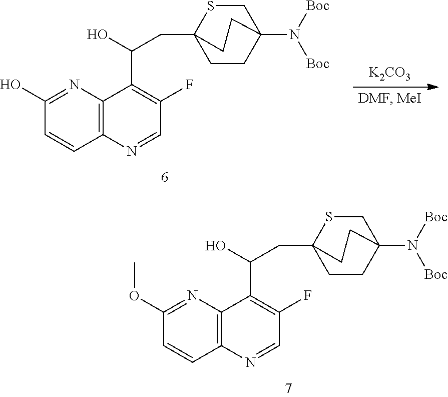

A mixture of 6 (25 mg, 0.05 mmol), CH₃I (14 mg, 0.1 mmol), K₂CO₃ (14 mg, 0.1 mmol) and DMF (4 mL) was stirred at room temperature for 12 h. Then the mixture was poured into water and extracted with EtOAc. The combined organic phases were washed with brine, dried over Na₂SO₄, filtered and the filtrate was concentrated. The crude compound was purified by pre-TLC (PE:EtOAc=5:1) (15 mg, yield: 54%).

### Preparation of Compound 8

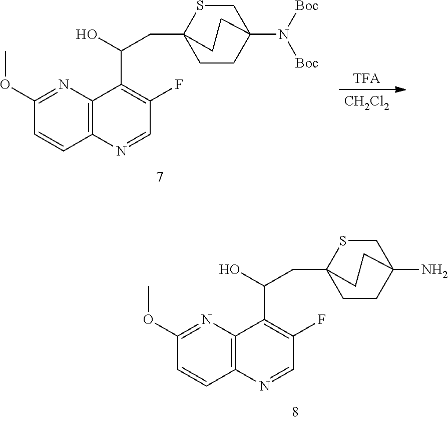

TFA (0.5 mL) was added dropwise to a mixture of 7 (20 mg, 0.04 mmol) in DCM (2 mL) at 0° C. under N₂. The mixture was stirred at room temperature for 2 h. Then the mixture was concentrated to give 4 (12 mg, 80%). The crude compound was used in next step directly.

### Preparation of Example 208

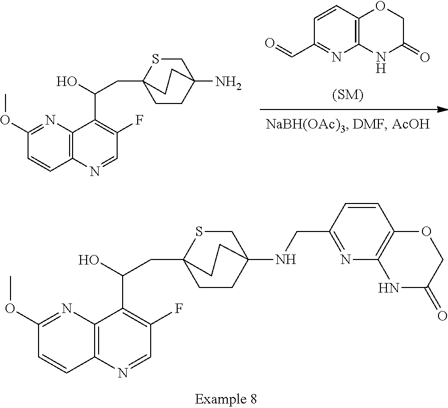 [SMILES: O=Cc1ccc2c(n1)NC(=O)CO2 ; COc1ccc2ncc(F)c(C(O)CC34CCC(N)(CC3)CS4)c2n1 ; COc1ccc2ncc(F)c(C(O)CC34CCC(NCc5ccc6c(n5)NC(=O)CO6)(CC3)CS4)c2n1]

A mixture of 8 (12 mg, 0.03 mmol), SM (11 mg, 0.06 mmol), AcOH (0.1 mL), DMF (2 mL) was stirred at room temperature overnight. Then NaBH(OAc)₃ (13 mg, 0.06 mmol) was added into the mixture. The resulting mixture was stirred at room temperature for another 2 h. Then the mixture was poured into water and adjusted to pH=8˜9 with aq. NaHCO₃. Then the mixture was extracted with EtOAc. The combined organic phases were washed with brine, dried over Na₂SO₄, filtered and the filtrate was concentrated. The crude compound was purified by pre-HPLC to give the product of Example 8.

¹H NMR 1 (400 MHz CD₃OD) δ 8.65 (s, 1H), 8.21 (d, J=8.8 Hz, 1H), 7.34 (d, J=8.0 Hz, 1H), 7.19 (d, J=8.8 Hz, 1H), 7.08 (d, J=8.0 Hz, 1H), 6.13˜6.10 (m, 1H), 4.67 (s, 2H), 4.20 (s, 2H), 4.11 (s, 3H), 3.05 (s, 2H), 2.55˜2.46 (m, 2H), 2.32˜2.11 (m, 5H), 2.08˜2.03 (m, 3H). MS m/z 526 (M+1)⁺.

### Example 209

### 1-(2-(3-Fluoro-6-(2-hydroxyethoxy)-1,5-naphthyridin-4-yl)-2-hydroxypropyl)-N-((3-oxo-3,4-dihydro-2H-pyrido[3,2-b][1,4]oxazin-6-yl)methyl)-2-oxabicyclo[2.2.2]octan-4-aminium chloride

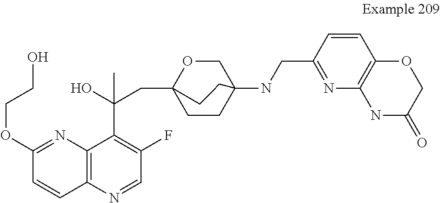 [SMILES: CC(O)(CC12CCC(NCc3ccc4c(n3)NC(=O)CO4)(CC1)CO2)c1c(F)cnc2ccc(OCCO)nc12]

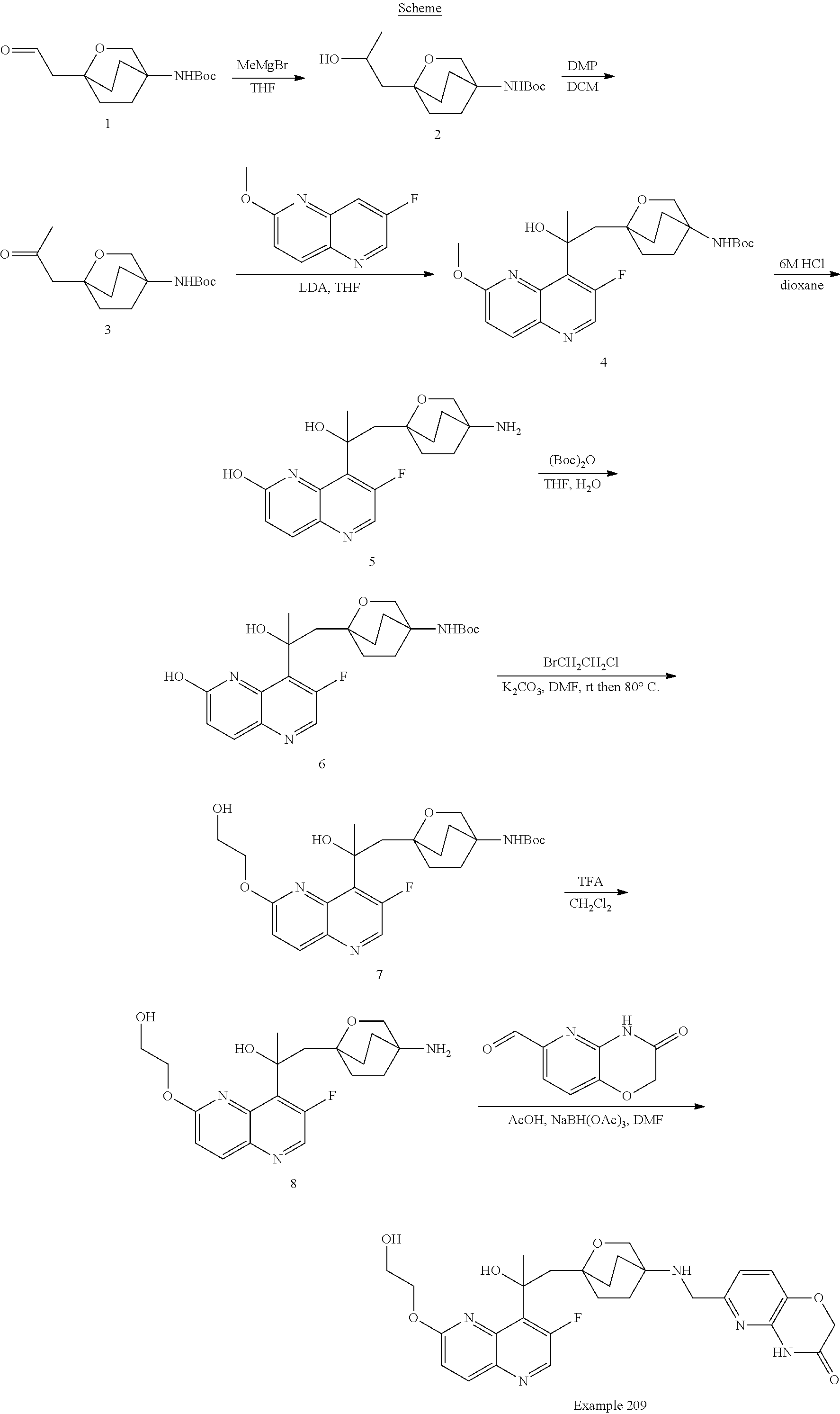

### Preparation of Compound 2

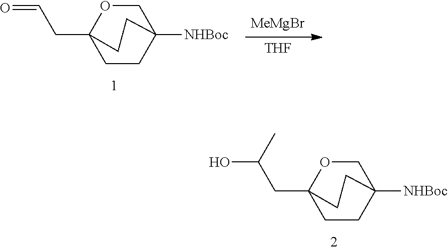 [SMILES: "MeMgBr" ; CC(C)(C)OC(=O)NC12CCC(CC=O)(CC1)OC2 ; CC(O)CC12CCC(NC(=O)OC(C)(C)C)(CC1)CO2 ; "THF"]

To a solution of 1 (800 mg, 2.9 mmol) in THF (20 mL) was added a solution of MeMgBr in Et₂O (3.0 M, 3 mL, 9.0 mmol) at −78° C. The mixture was stirred at −78° C. for 3 h. Then the reaction was quenched with water and extracted with EtOAc. The combined organic phases were washed with brine, dried over Na₂SO₄, filtered and the filtrate was concentrated. The crude product was purified by column chromatography on silica gel (PE:EtOAc=5:1) to give 2 (450 mg, yield: 53%).

¹H NMR (400 MHz CDCl₃) δ 4.04˜4.08 (m, 1H), 3.88˜3.95 (m, 2H), 2.10˜2.14 (m, 1H), 2.03˜2.07 (m, 2H), 1.76˜1.84 (m, 3H), 1.52˜1.66 (m, 4H), 1.38 (s, 9H), 1.10 (d, J=7.2 Hz, 3H).

### Preparation of Compound 3

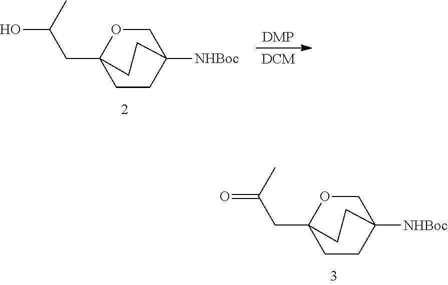

A mixture of 2 (450 mg, 1.6 mmol) and DMP (1.0 g, 2.3 mmol) in DCM (20 mL) was stirred overnight at room temperature. The reaction was quenched with saturated aq. NaHCO₃ and extracted with DCM. The combined organic phases were washed with brine, dried over Na₂SO₄, filtered and the filtrate was concentrated. The crude product was purified by column chromatography on silica gel (PE:EtOAc=10:1) to give 3 (350 mg, yield: 78%).

¹H NMR (400 MHz CDCl₃) δ 3.92 (s, 2H), 2.46 (s, 2H), 2.15 (s, 3H), 2.06˜2.10 (m, 3H), 1.79˜1.85 (m, 5H), 1.39 (s, 9H).

### Preparation of Compound 4

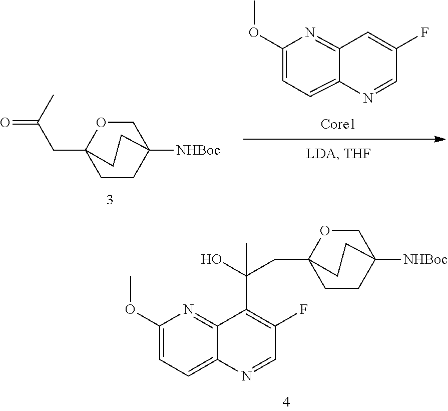 [SMILES: CC(=O)CC12CCC(NC(=O)OC(C)(C)C)(CC1)CO2 ; COc1ccc2ncc(F)cc2n1 ; COc1ccc2ncc(F)c(C(C)(O)CC34CCC(NC(=O)OC(C)(C)C)(CC3)CO4)c2n1]

To a solution of Core1 (100 mg, 0.56 mmol) in THF (5 mL) was added dropwise a solution of LDA in THF (0.5 M, 1.1 mL, 0.56 mmol) at −78° C. under N₂. Then the mixture was stirred at −78° C. for 1 h. A solution of 3 (80 mg, 0.28 mmol) in THF (1 mL) was added and the mixture was stirred at −78° C. for another 3 h. The reaction was quenched with water and extracted with EtOAc. The combined organic layers were washed with brine, dried over Na₂SO₄, filtered and the filtrate was concentrated. The crude product was purified by prep-TLC (PE:EtOAc=2:1) to give 4 (70 mg, yield: 54%).

¹H NMR (400 MHz CDCl₃) δ 8.57 (s, 1H), 8.24 (d, J=8.8 Hz, 1H), 7.10 (d, J=8.8 Hz, 1H), 4.01 (s, 3H), 3.52˜3.60 (m, 2H), 2.50˜2.54 (m, 1H), 1.80˜2.00 (m, 3H), 1.75 (s, 3H), 1.60˜1.69 (m, 6H), 1.34 (s, 9H).

### Preparation of Compound 5

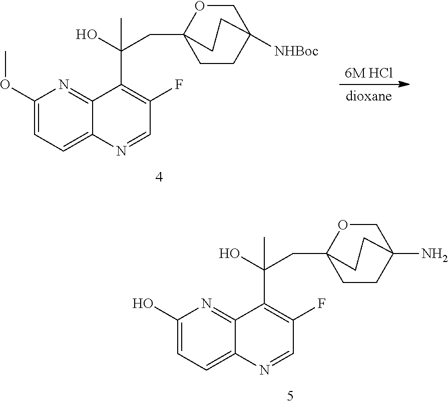 [SMILES: COc1ccc2ncc(F)c(C(C)(O)CC34CCC(NC(=O)OC(C)(C)C)(CC3)CO4)c2n1 ; "6M HCl" ; "dioxane" ; CC(O)(CC12CCC(N)(CC1)CO2)c1c(F)cnc2ccc(O)nc12]

A mixture of 4 (200 mg, 0.43 mmol) in conc. HCl/dioxane (3 mL3 mL) was stirred for 3 h at 80° C. The mixture was concentrated and the residue was used in next step directly.

### Preparation of Compound 6

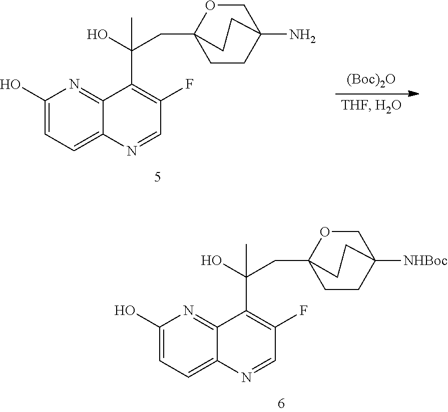

To a mixture of 5 (200 mg, 0.43 mmol) and NaHCO₃ (108 mg, 1.3 mmol) in THF/H₂O (3 mL3 mL) was added (Boc)₂O (140 mg, 0.64 mmol). Then the mixture was stirred overnight at room temperature. The mixture was extracted with EtOAc. The combined organic phases were washed with brine, dried over Na₂SO₄, filtered and the filtrate was concentrated. The crude product was purified by column chromatography on silica gel (DCM:MeOH=20:1) to give 6 (140 mg, yield: 73%).

¹H NMR (400 MHz CD₃OD) δ 8.34 (s, 1H), 8.00 (d, J=10.0 Hz, 1H), 6.82 (d, J=10.0 Hz, 1H), 3.58˜3.66 (m, 2H), 2.50˜2.54 (m, 1H), 1.85˜2.08 (m, 4H), 1.70 (s, 3H), 1.60˜1.69 (m, 5H), 1.33 (s, 9H).

### Preparation of Compound 7

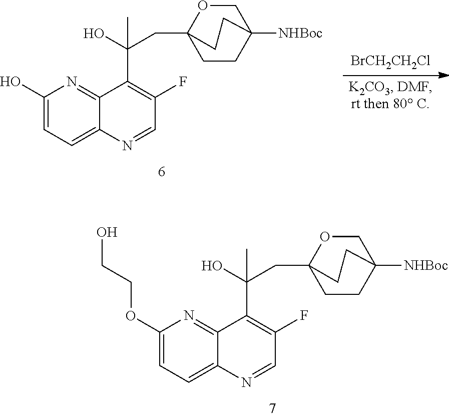

A mixture of 6 (70 mg, 0.15 mmol), BrCH₂CH₂Cl (65 mg, 0.45 mmol) and K₂CO₃ (82 mg, 0.6 mmol) in DMF (5 mL) was stirred for 10 h at 80° C. Then H₂O (1 mL) was added and the resulting mixture was stirred for 5 h at 80° C. The mixture was poured into water and extracted with EtOAc. The combined organic phases were washed with brine, dried over Na₂SO₄, filtered and the filtrate was concentrated. The crude product was purified by prep-TLC (DCM:MeOH=20:1) to give the product of 7 (20 mg, yield: 26%).

¹H NMR (400 MHz CDCl₃) δ 8.52 (s, 1H), 8.21 (d, J=9.2 Hz, 1H), 7.10 (d, J=9.2 Hz, 1H), 4.41˜4.45 (m, 2H), 3.99˜4.04 (m, 2H), 3.51 (s, 2H), 2.44˜2.48 (m, 1H), 1.93˜2.10 (m, 4H), 1.73 (s, 3H), 1.53˜1.68 (m, 5H), 1.28 (s, 9H).

### Preparation of Compound 8

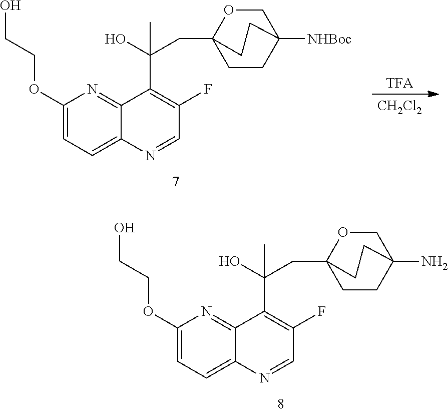 [SMILES: "TFA" ; CC(C)(C)OC(=O)NC12CCC(CC(C)(O)c3c(F)cnc4ccc(OCCO)nc34)(CC1)OC2 ; "CH2Cl2" ; CC(O)(CC12CCC(N)(CC1)CO2)c1c(F)cnc2ccc(OCCO)nc12]

A mixture of 7 (10 mg, 0.02 mmol) in DCM/TFA (0.5 mL2 mL) was stirred for 1 h at room temperature. Then the mixture was concentrated to give the crude product of 8. The crude product was used in next step directly.

### Preparation of Example 209

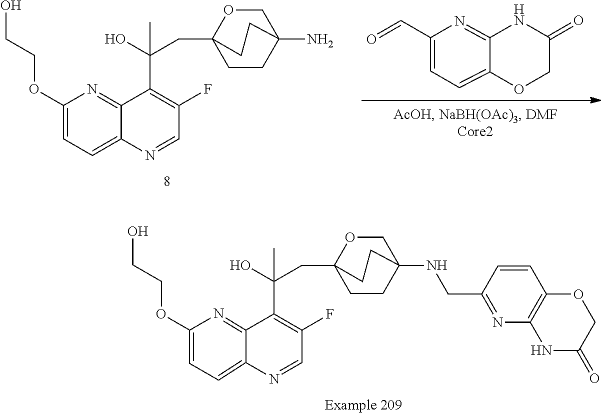 [SMILES: CC(O)(CC12CCC(N)(CC1)CO2)c1c(F)cnc2ccc(OCCO)nc12 ; O=Cc1ccc2c(n1)NC(=O)CO2 ; CC(O)(CC12CCC(NCc3ccc4c(n3)NC(=O)CO4)(CC1)CO2)c1c(F)cnc2ccc(OCCO)nc12]

A mixture of 8 (10 mg, 0.02 mmol), Corel (4 mg, 0.02 mmol), AcOH (0.05 mL) in DMF (2 mL) was stirred at room temperature overnight. Then NaBH(OAc)₃ (13 mg, 0.06 mmol) was added into the mixture. The resulting mixture was stirred at room temperature for another 1 h. Then the mixture was filtered and purified by prep-HPLC to give the product of Example 209 (6 mg, yield: 54%).

¹H NMR (400 MHz CD₃OD) δ 8.61 (s, 1H), 8.31 (d, J=9.2 Hz, 1H), 7.26˜7.30 (m, 2H), 7.00 (d, J=8.4 Hz, 1H), 4.64 (s, 2H), 4.41˜4.44 (m, 2H), 4.04 (s, 2H), 3.95˜3.97 (m, 2H), 3.58˜3.60 (m, 1H), 3.40˜3.42 (m, 1H), 2.59˜2.63 (m, 1H), 2.19˜2.23 (m, 1H), 1.78˜2.07 (m, 8H), 1.74 (s, 3H). MS m/z 554 (M+1)⁺.

### Example 210

### 1-(2-(3-Fluoro-6-(3-hydroxypropoxy)-1,5-naphthyridin-4-yl)-2-hydroxyethyl)-N-((3-oxo-3,4-dihydro-2H-pyrido[3,2-b][1,4]oxazin-6-yl)methyl)-2-oxabicyclo[2.2.2]octan-4-aminium chloride

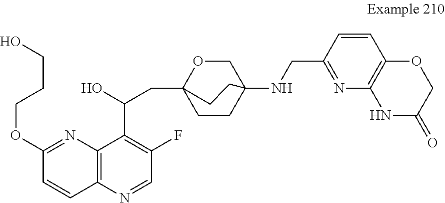 [SMILES: O=C1COc2ccc(CNC34CCC(CC(O)c5c(F)cnc6ccc(OCCCO)nc56)(CC3)OC4)nc2N1]

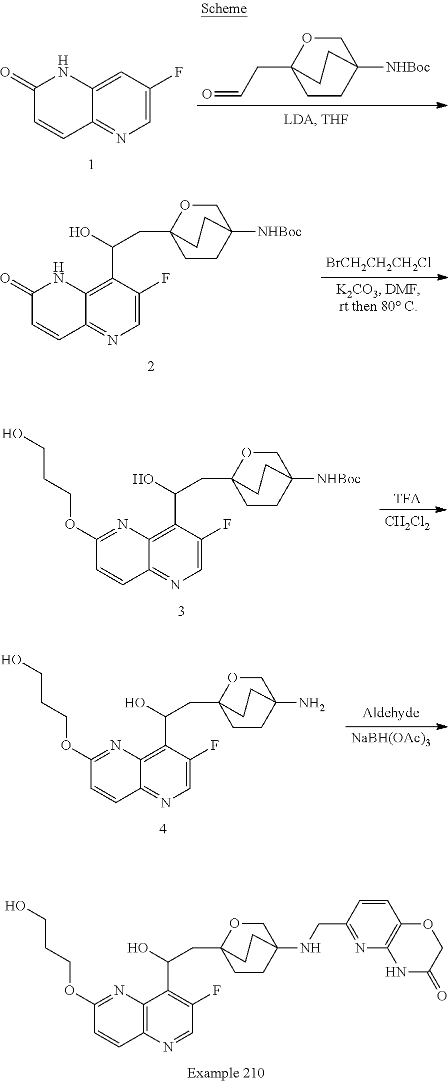

### Preparation of Compound 2

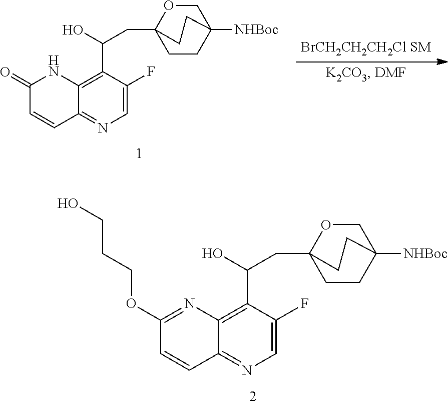

A mixture of 1 (90 mg, 0.21 mmol), K₂CO₃ (57 mg, 0.42 mmol) in DMF (4 mL) was stirred at room temperature for 1 h. Then SM (66 mg, 0.42 mmol) was added into the mixture. The mixture was stirred at 80° C. overnight. Then H₂O (1 mL) was added and the resulting mixture was stirred for 5 h at 80° C. After the reaction completed, the mixture was concentrated, diluted with water, extracted with EtOAc. The combined organic phases were washed with brine, dried over Na₂SO₄, filtered and the filtrate was concentrated. The residue was purified by column chromatography on silica gel (PE:EtOAc=1:1) to give 2 (30 mg, yield: 29%).

### Preparation of Compound 3

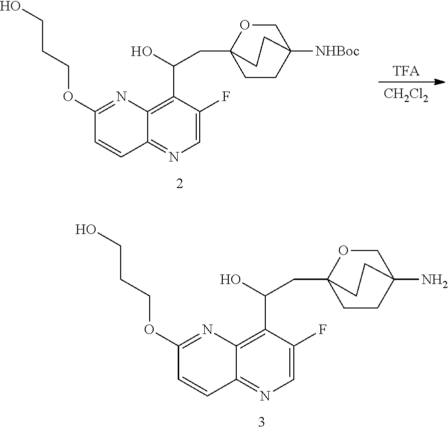 [SMILES: "TFA" ; CC(C)(C)OC(=O)NC12CCC(CC(O)c3c(F)cnc4ccc(OCCCO)nc34)(CC1)OC2 ; "CH2Cl2" ; NC12CCC(CC(O)c3c(F)cnc4ccc(OCCCO)nc34)(CC1)OC2]

2 (30 mg, 0.06 mmol) in CH₂Cl₂TFA (6 mL5:1) was stirred at room temperature for 1 h. Then the mixture was concentrated to give the product of 3. The crude compound was used in next step directly.

### Preparation of Example 210

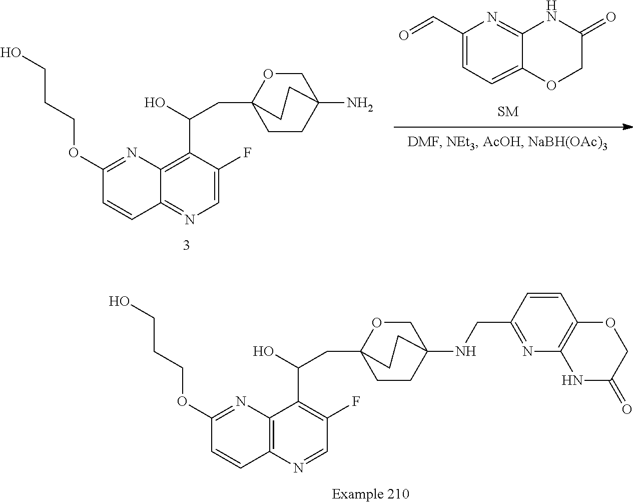 [SMILES: NC12CCC(CC(O)c3c(F)cnc4ccc(OCCCO)nc34)(CC1)OC2 ; O=Cc1ccc2c(n1)NC(=O)CO2 ; O=C1COc2ccc(CNC34CCC(CC(O)c5c(F)cnc6ccc(OCCCO)nc56)(CC3)OC4)nc2N1]

The mixture of 3 (24 mg, 0.06 mmol), DMF (4 mL) was stirred and adjusted to pH 7˜8 with Et₃N. Then SM (11 mg, 0.06 mmol) and AcOH (0.5 mL) were added into the mixture. The mixture was stirred at room temperature overnight. Then NaBH(OAc)₃ (30 mg) was added into the mixture. The resulting mixture was stirred at room temperature for another 0.5 h. The mixture was concentrated and the residue was purified by prep-HPLC to give Example 210 (10 mg, yield: 30%).

¹H NMR (400 MHz, CD₃OD) δ 8.64 (s, 1H), 8.23 (d, J=9.2 Hz, 1H), 7.35 (d, J=8.0 Hz, 1H), 7.19 (d, J=9.2 Hz, 1H), 7.07 (d, J=8.0 Hz, 1H), 6.03 (m, 1H), 4.68 (s, 2H), 4.62 (t, J=6.0 Hz, 2H), 4.17 (s, 2H), 3.90˜3.84 (m, 2H), 3.78 (t, J=6.0 Hz, 2H), 2.40˜1.97 (m, 12H). MS m/z 554 (M+1)⁺.

### Example 211

The following compound was prepared consistent with the methods described herein.

### 4-(2-Hydroxy-2-(4-((3-oxo-3,4-dihydro-2H-pyrido[3,2-b][1,4]oxazin-6-yl)methylamino)-2-oxabicyclo[2.2.2]octan-1-yl)ethyl)-6-methoxy-1,5-naphthyridine-3-carbonitrile Hydrochloride (Enantiomer A)

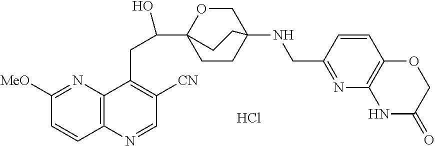 [SMILES: COc1ccc2ncc(C#N)c(CC(O)C34CCC(NCc5ccc6c(n5)NC(=O)CO6)(CC3)CO4)c2n1 ; Cl]

The title compound (75.9 mg) was prepared from 4-(2-(4-amino-2-oxabicyclo[2.2.2]octan-1-yl)-2-hydroxyethyl)-6-methoxy-1,5-naphthyridine-3-carbonitrile (70.0 mg, Enantiomer A) and 3-oxo-3,4-dihydro-2H-pyrido[3,2-b][1,4]oxazine-6-carbaldehyde (36.9 mg) in the same manner as described for Step 3 of EXAMPLE 1.

¹H NMR (DMSO-d₆) δ 1.83-2.18 (m, 8H), 3.04 (dd, J=12.2, 10.4 Hz, 1H), 3.74 (dd, J=12.2, 2.4 Hz, 1H), 3.83 (d, J=9.8 Hz, 1H), 3.93 (s, 2H), 4.05 (s, 3H), 4.10 (br, 2H), 4.69 (s, 2H), 4.82 (br, 1H), 7.25 (d, J=7.9 Hz, 1H), 7.42 (d, J=9.2 Hz, 1H), 7.45 (d, J=8.6 Hz, 1H), 8.34 (d, J=9.2 Hz, 1H), 8.98 (s, 1H), 9.39 (brs, 2H), 11.33 (s, 1H).

MS (ESI⁺) m/z: 517 (MH⁺) (as free base).

HRMS (ESI⁺) for C₂₇H₂₉N₆O₅ (MH⁺) (as free base): calcd, 517.21994. found, 517.22058.

### Preparation of Intermediates

### Step 1

### Preparation of 6-Methoxy-4-methyl-1,5-naphthyridine-3-carbonitrile

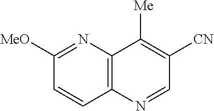 [SMILES: COc1ccc2ncc(C#N)c(C)c2n1]

A degassed mixture of 4-bromo-6-methoxy-1,5-naphthyridine-3-carbonitrile (528 mg), methylboronic acid (359 mg) and potassium carbonate (829 mg), tetrakis(triphenylphosphine)palladium (231 mg) and dioxane (2.4 mL) was stirred at 95° C. for 17 hours. After dilution of the mixture with dichloromethane, the mixture was washed with water and brine, dried over anhydrous sodium sulfate, filtered, and then concentrated in vacuo. Flash chromatography (silica, toluene:ethyl acetate=5:1) of the residue gave the title compound (235 mg).

¹H NMR (CDCl₃) δ 2.95 (s, 3H), 4.11 (s, 3H), 7.24 (d, J=9.2 Hz, 1H), 8.21 (d, J=9.2 Hz, 1H), 8.83 (s, 1H).

MS (EI⁺) m/z: 199 (M⁺).

HRMS (EI⁺) for C₁₁H₉N₃O (M⁺): calcd, 199.0746. found, 199.0756.

### Step 2

### Preparation of tert-Butyl 1-(2-(3-Cyano-6-methoxy-1,5-naphthyridin-4-yl)-1-hydroxyethyl)-2-oxabicyclo[2.2.2]octan-4-ylcarbamate

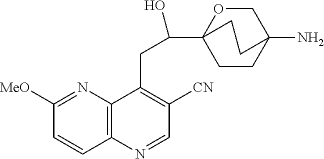 [SMILES: COc1ccc2ncc(C#N)c(CC(O)C34CCC(N)(CC3)CO4)c2n1]

The title compound (2.13 g) was prepared from 6-methoxy-4-methyl-1,5-naphthyridine-3-carbonitrile (4.37 g) in the same manner as described for Step 1 of EXAMPLE 20.

Enantiomer A: ¹H NMR (DMSO-d₆) δ 1.36 (s, 9H), 1.73-1.86 (m, 5H), 1.90-2.04 (m, 3H), 2.96-3.04 (m, 1H), 3.69-3.81 (m, 4H), 4.04 (s, 3H), 4.62 (d, J=6.1 Hz, 1H), 6.60 (br, 1H), 7.41 (d, J=9.1 Hz, 1H), 8.33 (d, J=9.1 Hz, 1H), 8.96 (s, 1H).

MS (ESI⁺) m/z: 455 (MH⁺).

HRMS (ESI⁺) for C₂₄H₃₁N₄O₅ (MH⁺): calcd, 455.22944. found, 455.22952.

Enantiomer B: ¹H NMR (DMSO-d₆) δ 1.36 (s, 9H), 1.73-1.86 (m, 5H), 1.88-2.00 (m, 3H), 2.96-3.03 (m, 1H), 3.68-3.81 (m, 4H), 4.04 (s, 3H), 4.63 (d, J=6.1 Hz, 1H), 6.62 (br, 1H), 7.41 (d, J=8.6 Hz, 1H), 8.33 (d, J=9.2 Hz, 1H), 8.96 (s, 1H).

MS (ESI⁺) m/z: 455 (MH⁺).

HRMS (ESI⁺) for C₂₄H₃₁N₄O₅ (MH⁺): calcd, 455.22944. found, 455.22904.

### Step 3

### Preparation of 4-(2-(4-Amino-2-oxabicyclo[2.2.2]octan-1-yl)-2-hydroxyethyl)-6-methoxy-1,5-naphthyridine-3-carbonitrile

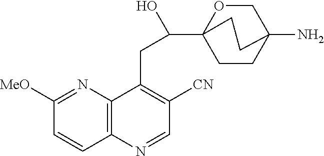 [SMILES: COc1ccc2ncc(C#N)c(CC(O)C34CCC(N)(CC3)CO4)c2n1]

The title compound (145 mg, Enantiomer A, 145 mg, Enantiomer B) was prepared from tert-butyl 1-(2-(3-cyano-6-methoxy-1,5-naphthyridin-4-yl)-1-hydroxyethyl)-2-oxabicyclo[2.2.2]octan-4-ylcarbamate (200 mg, Enantiomer A, 200 mg, Enantiomer B) in the same manner as described for Step 2 of EXAMPLE 32.

Enantiomer A: ¹H NMR (DMSO-d₆) δ 1.31 (s, 2H), 1.51-1.64 (m, 4H), 1.69-1.84 (m, 3H), 1.91-2.00 (m, 1H), 3.02 (dd, J=12.2, 10.4 Hz, 1H), 3.41-3.48 (m, 2H), 3.71 (dd, J=12.2, 3.1 Hz, 1H), 3.78 (ddd, J=10.4, 5.5, 3.1 Hz, 1H), 4.04 (s, 3H), 4.55 (d, J=5.5 Hz, 1H), 7.41 (d, J=9.2 Hz, 1H), 8.32 (d, J=9.2 Hz, 1H), 8.96 (s, 1H).

MS (ESI⁺) m/z: 355 (MH⁺).

HRMS (ESI⁺) for C₁₉H₂₃N₄O₃ (MH⁺): calcd, 355.17701. found, 355.17717.

Enantiomer B: ¹H NMR (DMSO-d₆) δ 1.31 (s, 2H), 1.47-1.65 (m, 4H), 1.71-1.86 (m, 3H), 1.90-2.01 (m, 1H), 3.02 (dd, J=12.2, 10.4 Hz, 1H), 3.42-3.47 (m, 2H), 3.71 (dd, J=12.2, 3.1 Hz, 1H), 3.78 (ddd, J=9.8, 5.5, 3.1 Hz, 1H), 4.04 (s, 3H), 4.54 (d, J=5.5 Hz, 1H), 7.41 (d, J=9.2 Hz, 1H), 8.33 (d, J=9.2 Hz, 1H), 8.96 (s, 1H).

MS (ESI) m/z: 355 (MH⁺).

HRMS (ESI⁺) for C₁₉H₂₃N₄O₃ (MH⁺): calcd, 355.17701. found, 355.17688.

### Example 212

### 4-(2-Hydroxy-2-(4-((3-oxo-3,4-dihydro-2H-pyrido[3,2-b][1,4]oxazin-6-yl)methylamino)-2-oxabicyclo[2.2.2]octan-1-yl)ethyl)-6-methoxy-1,5-naphthyridine-3-carbonitrile Hydrochloride (Enantiomer B)

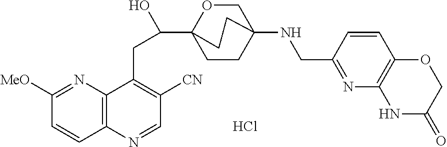 [SMILES: COc1ccc2ncc(C#N)c(CC(O)C34CCC(NCc5ccc6c(n5)NC(=O)CO6)(CC3)CO4)c2n1 ; Cl]

The title compound (74.6 mg) was prepared from 4-(2-(4-amino-2-oxabicyclo[2.2.2]octan-1-yl)-2-hydroxyethyl)-6-methoxy-1,5-naphthyridine-3-carbonitrile (70.0 mg, Enantiomer B) and 3-oxo-3,4-dihydro-2H-pyrido[3,2-b][1,4]oxazine-6-carbaldehyde (36.9 mg) in the same manner as described for Step 3 of EXAMPLE 1.

¹H NMR (DMSO-d₆) δ 1.83-2.16 (m, 8H), 3.04 (dd, J=12.2, 9.8 Hz, 1H), 3.74 (dd, J=12.2, 2.4 Hz, 1H), 3.83 (d, J=9.8 Hz, 1H), 3.93 (s, 2H), 4.05 (s, 3H), 4.10 (br, 2H), 4.69 (s, 2H), 4.82 (br, 1H), 7.25 (d, J=7.9 Hz, 1H), 7.42 (d, J=9.2 Hz, 1H), 7.45 (d, J=7.9 Hz, 1H), 8.34 (d, J=8.6 Hz, 1H), 8.98 (s, 1H), 9.39 (br, 2H), 11.33 (s, 1H).

MS (ESI⁺) m/z: 517 (MH⁺) (as free base).

HRMS (ESI⁺) for C₂₇H₂₉N₆O₅ (MH⁺) (as free base): calcd, 517.21994. found, 517.21903.

### Example 213

The following compound was prepared consistent with the methods described herein.

6-(2-Hydroxyethoxy)-4-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-3-carbonitrile

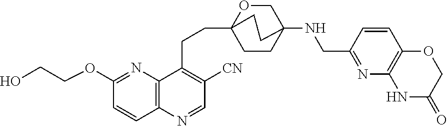 [SMILES: N#Cc1cnc2ccc(OCCO)nc2c1CCC12CCC(NCc3ccc4c(n3)NC(=O)CO4)(CC1)CO2]

The title compound (94.8 mg) was prepared from 4-(2-(4-amino-2-oxabicyclo[2.2.2]octan-1-yl)ethyl)-6-(2-hydroxyethoxy)-1,5-naphthyridine-3-carbonitrile (67.0 mg) and 3-oxo-3,4-dihydro-2H-pyrido[3,2-b][1,4]oxazine-6-carbaldehyde (35.0 mg) in the same manner as described for Step 3 of EXAMPLE 1.

¹H NMR (DMSO-d₆) δ 1.60-1.93 (m, 10H), 3.26-3.32 (m, 2H), 3.58 (s, 2H), 3.62 (s, 2H), 3.80 (q, J=4.9 Hz, 2H), 4.48 (t, J=4.9 Hz, 2H), 4.59 (s, 2H), 4.92 (t, J=5.2 Hz, 1H), 7.01 (d, J=8.6 Hz, 1H), 7.28 (d, J=7.6 Hz, 1H), 7.41 (d, J=9.2 Hz, 1H), 8.33 (d, J=9.2 Hz, 1H), 8.97 (s, 1H), 11.16 (br, 1H).

MS (ESI⁺) m/z: 531 (MH⁺).

HRMS (ESI⁺) for C₂₈H₃₁N₆O₅ (MH⁺): calcd, 531.23559. found, 531.23585.

### Preparation of Intermediates

### Step 1

### Preparation of tert-Butyl 1-(2-(3-Cyano-6-(2-(tetrahydro-2H-pyran-2-yloxy)ethoxy)-1,5-naphthyridin-4-yl)ethyl)-2-oxabicyclo[2.2.2]octan-4-ylcarbamate

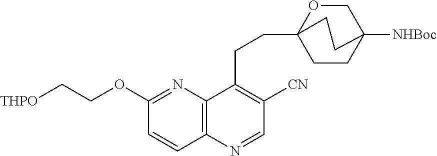

The title compound (51.5 mg) was prepared from tert-butyl 1-(2-(3-cyano-6-hydroxy-1,5-naphthyridin-4-yl)ethyl)-2-oxabicyclo[2.2.2]octan-4-ylcarbamate (50.0 mg) and 2-(2-bromoethoxy)tetrahydro-2H-pyran (74.0 mg) in the same manner as described for Step 1 of EXAMPLE 32.

¹H NMR (CDCl₃) δ 1.43 (s, 9H), 1.54-1.65 (m, 4H), 1.75-1.92 (m, 8H), 2.01-2.11 (m, 4H), 3.46-3.40 (m, 2H), 3.52-3.59 (m, 1H), 3.86-3.93 (m, 2H), 3.95 (s, 2H), 4.14-4.18 (m, 1H), 4.30 (br, 1H), 4.67-4.73 (m, 3H), 7.27 (d, J=9.2 Hz, 1H), 8.21 (d, J=9.2 Hz, 1H), 8.81 (s, 1H).

MS (CI⁺) m/z: 553 (MH⁺).

HRMS (ESI⁺) for C₃₀H₄₁N₄O₆ (MH⁺): calcd, 553.3026. found, 553.3018.

### Step 2

### Preparation of 4-(2-(4-Amino-2-oxabicyclo[2.2.2]octan-1-yl)ethyl)-6-(2-hydroxyethoxy)-1,5-naphthyridine-3-carbonitrile

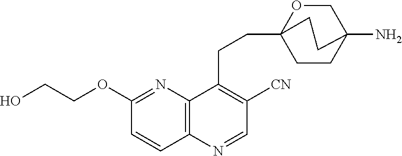 [SMILES: N#Cc1cnc2ccc(OCCO)nc2c1CCC12CCC(N)(CC1)CO2]

The title compound (67.0 mg) was prepared from tert-butyl 1-(2-(3-cyano-6-(2-(tetrahydro-2H-pyran-2-yloxy)ethoxy)-1,5-naphthyridin-4-yl)ethyl)-2-oxabicyclo[2.2.2]octan-4-ylcarbamate (100 mg) in the same manner as described for Step 2 of EXAMPLE 32.

¹H NMR (CDCl₃) δ 1.66-1.80 (m, 8H), 2.00-2.05 (m, 2H), 3.39-3.44 (m, 2H), 3.67 (s, 2H), 4.01 (t, J=5.5 Hz, 1H), 4.71 (t, J=5.5 Hz, 1H), 7.25 (d, J=8.6 Hz, 1H), 8.23 (d, J=9.2 Hz, 1H), 8.82 (s, 1H).

MS (ESI⁺) m/z: 369 (MH⁺).

HRMS (ESI⁺) for C₂₀H₂₅N₄O₃ (MH⁺): calcd, 369.19266. found, 369.19348.

### Example 214

The following compound was prepared consistent with the methods described herein.

### 6-(3-Hydroxypropoxy)-4-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-3-carbonitrile

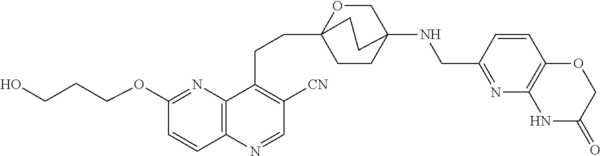 [SMILES: N#Cc1cnc2ccc(OCCCO)nc2c1CCC12CCC(NCc3ccc4c(n3)NC(=O)CO4)(CC1)CO2]

The title compound (87.8 mg) was prepared from 4-(2-(4-amino-2-oxabicyclo[2.2.2]octan-1-yl)ethyl)-6-(3-hydroxypropoxy)-1,5-naphthyridine-3-carbonitrile (67.0 mg) and 3-oxo-3,4-dihydro-2H-pyrido[3,2-b][1,4]oxazine-6-carbaldehyde (34.0 mg) in the same manner as described for Step 3 of EXAMPLE 1.

¹H NMR (DMSO-d₆) δ 1.64-1.73 (m, 8H), 1.88-1.97 (m, 4H), 3.27-3.34 (m, 2H), 3.56-3.63 (m, 6H), 4.52-4.59 (m, 5H), 7.01 (d, J=7.9 Hz, 1H), 7.28 (d, J=7.9 Hz, 1H), 7.40 (d, J=9.2 Hz, 1H), 8.32 (d, J=8.6 Hz, 1H), 8.97 (s, 1H), 11.16 (brs, 1H).

MS (ESI⁺) m/z: 545 (MH⁺).

HRMS (ESI⁺) for C₂₉H₃₃N₆O₅ (MH⁺): calcd, 545.25124. found, 545.25079.

### Preparation of Intermediates

### Step 1

### Preparation of tert-Butyl 1-(2-(3-Cyano-6-(3-(tetrahydro-2H-pyran-2-yloxy)propoxy)-1,5-naphthyridin-4-yl)ethyl)-2-oxabicyclo[2.2.2]octan-4-ylcarbamate

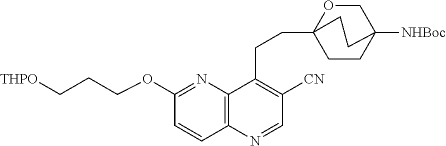

The title compound (103 mg) was prepared from tert-butyl 1-(2-(3-cyano-6-hydroxy-1,5-naphthyridin-4-yl)ethyl)-2-oxabicyclo[2.2.2]octan-4-ylcarbamate (100 mg) and 2-(3-bromopropoxy)tetrahydro-2H-pyran (70 uL) in the same manner as described for Step 1 of EXAMPLE 32.

¹H NMR (CDCl₃) δ 1.43 (s, 9H), 1.50-1.65 (m, 4H), 1.73-1.89 (m, 8H), 2.01-2.17 (m, 6H), 3.36-3.41 (m, 2H), 3.50-3.53 (m, 1H), 3.57-3.63 (m, 1H), 3.84-3.89 (m, 1H), 3.94 (s, 2H), 3.95-3.99 (m, 1H), 4.29 (br, 1H), 4.60-4.63 (m, 3H), 7.20 (d, J=9.2 Hz, 1H), 8.19 (d, J=9.2 Hz, 1H), 8.80 (s, 1H).

MS (CI⁺) m/z: 567 (MH⁺).

HRMS (ESI⁺) for C₃₁H₄₃N₄O₆ (MH⁺): calcd, 567.3138. found, 567.3203.

### Step 2

### Preparation of 4-(2-(4-Amino-2-oxabicyclo[2.2.2]octan-1-yl)ethyl)-6-(3-hydroxypropoxy)-1,5-naphthyridine-3-carbonitrile

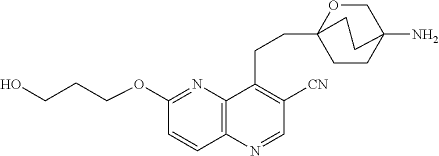 [SMILES: N#Cc1cnc2ccc(OCCCO)nc2c1CCC12CCC(N)(CC1)CO2]

The title compound (68.0 mg) was prepared from tert-butyl 1-(2-(3-cyano-6-(3-(tetrahydro-2H-pyran-2-yloxy)propoxy)-1,5-naphthyridin-4-yl)ethyl)-2-oxabicyclo[2.2.2]octan-4-ylcarbamate (99.0 mg) in the same manner as described for Step 2 of EXAMPLE 32.

¹H NMR (DMSO-d₆) δ 1.57-1.75 (m, 9H), 1.84-1.98 (m, 4H), 3.16-3.28 (m, 2H), 3.48 (s, 2H), 3.58 (q, J=5.5 Hz, 2H), 4.52-4.58 (m, 3H), 7.39 (d, J=9.2 Hz, 1H), 8.31 (d, J=9.2 Hz, 1H), 8.96 (s, 1H).

MS (ESI⁺) m/z: 383 (MH⁺).

HRMS (ESI⁺) for C₂₁H₂₇N₄O₃ (MH⁺): calcd, 383.20831. found, 383.20873.

### Example 215

The following compound was prepared consistent with the methods described herein.

### 6-((1-(2-(6-(((1S,3R,4S)-3,4-Dihydroxycyclopentyl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

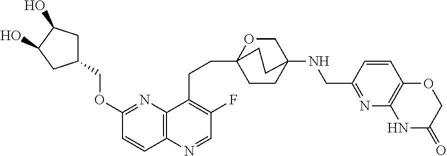 [SMILES: O=C1COc2ccc(CNC34CCC(CCc5c(F)cnc6ccc(OC[C@@H]7C[C@@H](O)[C@@H](O)C7)nc56)(CC3)OC4)nc2N1]

The title compound (29.5 mg) was prepared from (1R,2S,4s)-4-((8-(2-(4-amino-2-oxabicyclo[2.2.2]octan-1-yl)ethyl)-7-fluoro-1,5-naphthyridin-2-yloxy)methyl)cyclopentane-1,2-diol (36.0 mg) and 3-oxo-3,4-dihydro-2H-pyrido[3,2-b][1,4]oxazine-6-carbaldehyde (16.3 mg) in the same manner as described for Step 3 of EXAMPLE 1.

¹H NMR (CDCl₃) δ 1.74-1.84 (m, 10H), 1.97-2.07 (m, 4H), 2.89-2.93 (m, 1H), 3.15-3.19 (m, 2H), 3.78 (s, 4H), 4.20-4.28 (m, 2H), 4.38 (d, J=6.7 Hz, 2H), 4.62 (s, 2H), 6.94 (d, J=7.9 Hz, 1H), 7.04 (d, J=9.2 Hz, 1H), 7.20 (d, J=8.6 Hz, 1H), 8.16 (d, J=9.2 Hz, 1H), 8.59 (s, 1H).

MS (ESI⁺) m/z: 594 (MH⁺).

HRMS (ESI⁺) for C₃₁H₃₇FN₅O₆ (MH⁺): calcd, 594.27279. found, 594.27306.

### Preparation of Intermediates

### Step 1

### Preparation of tert-Butyl 1-(2-(6-(((3aR,5s,6aS)-2,2-Dimethyltetrahydro-3aH-cyclopenta[d][1,3]dioxol-5-yl)methoxy)-3-fluoro-1,5-naphthyridin-4-yl)ethyl)-2-oxabicyclo[2.2.2]octan-4-ylcarbamate

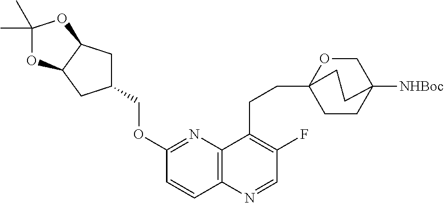 [SMILES: CC(C)(C)OC(=O)NC12CCC(CCc3c(F)cnc4ccc(OC[C@H]5C[C@@H]6OC(C)(C)O[C@@H]6C5)nc34)(CC1)OC2]

The title compound (123 mg) was prepared from tert-butyl 1-(2-(3-fluoro-6-hydroxy-1,5-naphthyridin-4-yl)ethyl)-2-oxabicyclo[2.2.2]octan-4-ylcarbamate (100 mg) and (3aR,5s,6aS)-5-(bromomethyl)-2,2-dimethyltetrahydro-3aH-cyclopenta[d][1,3]dioxole (74 mg) in the same manner as described for Step 1 of EXAMPLE 32.

¹H NMR (CDCl₃) δ 1.28 (s, 3H), 1.31-1.39 (m, 2H), 1.43 (s, 9H), 1.47 (s, 3H), 1.40-1.47 (br, 2H), 1.71-1.92 (m, 6H), 1.99-2.13 (m, 6H), 2.71-2.77 (m, 1H), 3.13-3.17 (m, 2H), 3.95 (s, 2H), 4.30 (br, 1H), 4.46 (d, J=6.1 Hz, 2H), 4.71 (dd, J=8.6, 4.3 Hz, 2H), 7.03 (d, J=9.2 Hz, 1H), 8.15 (d, J=9.2 Hz, 1H), 8.58 (s, 1H).

### Step 2

### Preparation of (1R,2S,4s)-4-((8-(2-(4-Amino-2-oxabicyclo[2.2.2]octan-1-yl)ethyl)-7-fluoro-1,5-naphthyridin-2-yloxy)methyl)cyclopentane-1,2-diol

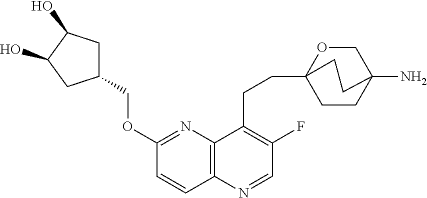 [SMILES: NC12CCC(CCc3c(F)cnc4ccc(OC[C@@H]5C[C@@H](O)[C@@H](O)C5)nc34)(CC1)OC2]

The title compound (41.0 mg) was prepared from tert-butyl 1-(2-(6-(((3aR,5s,6aS)-2,2-dimethyltetrahydro-3aH-cyclopenta[d][1,3]dioxol-5-yl)methoxy)-3-fluoro-1,5-naphthyridin-4-yl)ethyl)-2-oxabicyclo[2.2.2]octan-4-ylcarbamate (108 mg) in the same manner as described for Step 2 of EXAMPLE 1.

¹H NMR (CDCl₃) δ 1.65-1.82 (m, 10H), 1.96-2.06 (m, 4H), 2.87-2.96 (m, 1H), 3.14-3.18 (m, 2H), 3.65 (s, 2H), 4.21-4.25 (m, 2H), 4.37 (d, J=6.1 Hz, 2H), 7.04 (d, J=8.6 Hz, 1H), 8.16 (d, J=9.2 Hz, 1H), 8.16 (s, 1H).

MS (ESI⁺) m/z: 432 (MH⁺).

HRMS (ESI⁺) for C₂₃H₃₁FN₃O₄ (MH⁺): calcd, 432.22986. found, 432.22971.

### Example 216

The following compound was prepared consistent with the methods described herein.

### 8-(2-(4-((3-Oxo-3,4-dihydro-2H-pyrido[3,2-b][1,4]oxazin-6-yl)methylamino)-2-oxabicyclo[2.2.2]octan-1-yl)ethyl)-1,5-naphthyridine-2,7-dicarbonitrile

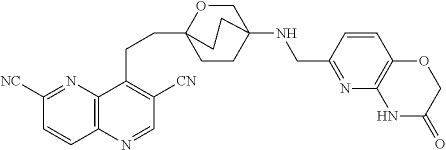 [SMILES: N#Cc1ccc2ncc(C#N)c(CCC34CCC(NCc5ccc6c(n5)NC(=O)CO6)(CC3)CO4)c2n1]

The title compound (77.8 mg) was prepared from 8-(2-(4-amino-2-oxabicyclo[2.2.2]octan-1-yl)ethyl)-1,5-naphthyridine-2,7-dicarbonitrile (70.0 mg) and 3-oxo-3,4-dihydro-2H-pyrido[3,2-b][1,4]oxazine-6-carbaldehyde (37.5 mg) in the same manner as described for Step 3 of EXAMPLE 1.

¹H NMR (DMSO-d₆) δ 1.57-1.78 (m, 8H), 1.84-1.99 (m, 2H), 3.38-3.45 (m, 2H), 3.53 (s, 2H), 3.62 (s, 2H), 4.59 (s, 2H), 7.00 (d, J=7.9 Hz, 1H), 7.27 (d, J=8.6 Hz, 1H), 8.44 (d, J=8.6 Hz, 1H), 8.76 (d, J=8.6 Hz, 1H), 9.32 (s, 1H), 11.16 (s, 1H).

MS (ESI⁺) m/z: 496 (MH⁺).

HRMS (ESI⁺) for C₂₇H₂₆N₇O₃ (MH⁺): calcd, 496.20971. found, 496.20947.

### Preparation of Intermediates

### Step 1

### Preparation of tert-Butyl 1-(2-(3-Cyano-6-hydroxy-1,5-naphthyridin-4-yl)ethyl)-2-oxabicyclo[2.2.2]octan-4-ylcarbamate

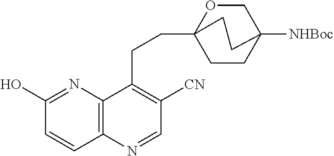 [SMILES: CC(C)(C)OC(=O)NC12CCC(CCc3c(C#N)cnc4ccc(O)nc34)(CC1)OC2]

A mixture of 4-(2-(4-amino-2-oxabicyclo[2.2.2]octan-1-yl)ethyl)-6-methoxy-1,5-naphthyridine-3-carbonitrile (1.21 g) and hydrogen bromide-acetic acid solution (25 mL) was stirred at room temperature for 2.5 hours, then concentrated in vacuo. To a solution of the resulting residue in tetrahydrofuran (30 mL) and saturated sodium hydrogencarbonate sok\lution (30 mL) was added di-tert-butyl dicarbonate (705 mg), the mixture was stirred at 60° C. for 18 hours. After dilution of the mixture with water, the mixture was extracted with ethyl acetate. The organic extracts were washed with brine, dried over anhydrous sodium sulfate, filtered, and then concentrated in vacuo. Flash chromatography (silica, chloroform:methanol=30:1) of the residue gave the title compound.

¹H NMR (CDCl₃) δ 1.44 (s, 9H), 1.68-1.72 (m, 2H), 1.78 (t, J=7.3 Hz, 2H), 1.87-2.00 (m, 4H), 2.16-2.17 (m, 2H), 3.10 (t, J=7.3 Hz, 2H), 4.22 (s, 2H), 4.34 (br, 1H), 6.95 (d, J=9.8 Hz, 1H), 7.92 (d, J=9.8 Hz, 1H), 8.60 (s, 1H).

MS (ESI⁺) m/z: 425 (MH⁺).

HRMS (ESI⁺) for C₂₃H₂₉N₄O₄ (MH⁺): calcd, 425.21888. found, 425.21854.

### Step 2

### Preparation of 8-(2-(4-(tert-Butoxycarbonylamino)-2-oxabicyclo[2.2.2]octan-1-yl)ethyl)-7-cyano-1,5-naphthyridin-2-yl Trifluoromethanesulfonate

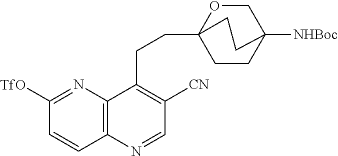 [SMILES: CC(C)(C)OC(=O)NC12CCC(CCc3c(C#N)cnc4ccc(OS(=O)(=O)C(F)(F)F)nc34)(CC1)OC2]

The title compound (186 mg) was prepared from tert-butyl 1-(2-(3-cyano-6-hydroxy-1,5-naphthyridin-4-yl)ethyl)-2-oxabicyclo[2.2.2]octan-4-ylcarbamate (200 mg) in the same manner as described for Step 1 of EXAMPLE 28.

¹H NMR (DMSO-d₆) δ 1.35 (s, 9H), 1.66-1.71 (m, 4H), 1.78-1.98 (m, 6H), 3.26-3.28 (m, 2H), 3.74 (s, 2H), 6.60 (br, 1H), 8.08 (d, J=9.2 Hz, 1H), 8.84 (d, J=8.6 Hz, 1H), 9.28 (s, 1H).

MS (ESI⁺) m/z: 557 (MH⁺).

HRMS (ESI⁺) for C₂₄H₂₈F₃N₄O₆S (MH⁺): calcd, 557.16816. found, 557.16873.

### Step 3

### Preparation of tert-Butyl 1-(2-(3,6-Dicyano-1,5-naphthyridin-4-yl)ethyl)-2-oxabicyclo[2.2.2]octan-4-ylcarbamate

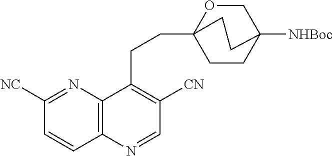 [SMILES: CC(C)(C)OC(=O)NC12CCC(CCc3c(C#N)cnc4ccc(C#N)nc34)(CC1)OC2]

The title compound (104 mg) was prepared from 8-(2-(4-(tert-butoxycarbonylamino)-2-oxabicyclo[2.2.2]octan-1-yl)ethyl)-7-cyano-1,5-naphthyridin-2-yltrifluoromethanesulfonate (183 mg) in the same manner as described for Step 1 of EXAMPLE 31

¹H NMR (CDCl₃) δ 1.43 (s, 9H), 1.76-1.92 (m, 6H), 2.06-2.11 (m, 4H), 3.52-3.56 (m, 2H), 3.87 (s, 2H), 4.28 (br, 1H), 8.03 (d, J=8.6 Hz, 1H), 8.58 (d, J=8.6 Hz, 1H), 9.08 (s, 1H).

MS (ESI⁺) m/z: 434 (MH⁺).

HRMS (ESI⁺) for C₂₄H₂₈N₅O₃ (MH⁺): calcd, 434.21921. found, 434.21941.

### Step 4

### Preparation of 8-(2-(4-Amino-2-oxabicyclo[2.2.2]octan-1-yl)ethyl)-1,5-naphthyridine-2,7-dicarbonitrile

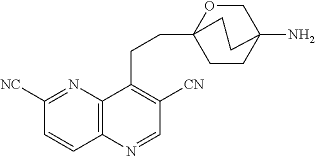 [SMILES: N#Cc1ccc2ncc(C#N)c(CCC34CCC(N)(CC3)CO4)c2n1]

The title compound (74.0 mg) was prepared from tert-butyl 1-(2-(3,6-dicyano-1,5-naphthyridin-4-yl)ethyl)-2-oxabicyclo[2.2.2]octan-4-ylcarbamate (100 mg) in the same manner as described for Step 2 of EXAMPLE 32.

¹H NMR (CDCl₃) δ 1.65-1.88 (m, 8H), 2.04-2.11 (m, 2H), 3.53-3.57 (m, 4H), 8.03 (d, J=8.6 Hz, 1H), 8.58 (d, J=8.6 Hz, 1H), 9.09 (s, 1H).

MS (ESI⁺) m/z: 334 (MH⁺).

HRMS (ESI⁺) for C₁₉H₂₀N₅O (MH⁺): calcd, 334.16678. found, 334.16643.

### Example 217

### HCl Salt of Example 54

### 6-((1-(1-Hydroxy-2-(7-methoxy-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one Hydrochloride

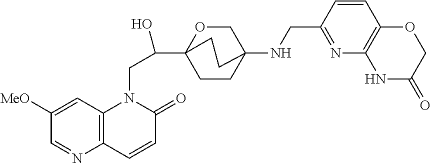 [SMILES: COc1cnc2ccc(=O)n(CC(O)C34CCC(NCc5ccc6c(n5)NC(=O)CO6)(CC3)CO4)c2c1]

¹H NMR (DMSO-d₆) δ 1.88-2.11 (m, 8H), 3.62 (br, 1H), 3.93 (s, 3H), 3.95 (s, 2H), 4.11 (s, 2H), 4.26-4.36 (m, 2H), 4.69 (s, 2H), 5.11 (d, J=6.1 Hz, 1H), 6.66 (d, J=9.8 Hz, 1H), 7.21 (d, J=7.9 Hz, 1H), 7.45 (d, J=7.9 Hz, 1H), 7.49 (d, J=2.4 Hz, 1H), 7.86 (d, J=9.8 Hz, 1H), 8.26 (d, J=2.4 Hz, 1H), 9.27 (br, 2H), 11.33 (s, 1H).

MS (ESI⁺) m/z: 508 (MH⁺) (as free base).

HRMS (ESI⁺) for C₂₆H₃₀N₅O₆ (MH⁺) (as free base): calcd, 508.21961. found, 506.21944.

### Example 218

The following compound was prepared consistent with the methods described herein.

### 6-((1-(2-(6-((1-Aminocyclopropyl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

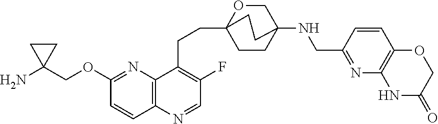 [SMILES: NC1(COc2ccc3ncc(F)c(CCC45CCC(NCc6ccc7c(n6)NC(=O)CO7)(CC4)CO5)c3n2)CC1]

The title compound (47.7 mg) was prepared from benzyl 1-((7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropylcarbamate (75.0 mg) in the same manner as described for Step 4 of EXAMPLE 38.

¹H NMR (DMSO-d₆) δ 0.57-0.60 (m, 4H), 1.54-1.76 (m, 8H), 1.78-1.95 (m, 3H), 3.00-3.12 (m, 2H), 3.58 (s, 2H), 3.63 (s, 2H), 4.37 (s, 2H), 4.59 (s, 2H), 7.01 (d, J=8.6 Hz, 1H), 7.25 (d, J=9.2 Hz, 1H), 7.28 (d, J=7.9 Hz, 1H), 8.25 (d, J=8.6 Hz, 1H), 8.73 (s, 1H), 11.18 (br, 1H).

MS (ESI⁺) m/z: 549 (MH⁺).

HRMS (ESI⁺) for C₂₉H₃₄FN₆O₄ (MH⁺): calcd, 549.26256. found, 549.26219.

### Preparation of Intermediates

### Step 1

### Preparation of tert-Butyl 1-(2-(3-Fluoro-6-((1-benzyloxycarbonylaminocyclopropyl)methoxy)-1,5-naphthyridin-4-yl)ethyl)-2-oxabicyclo[2.2.2]octan-4-ylcarbamate

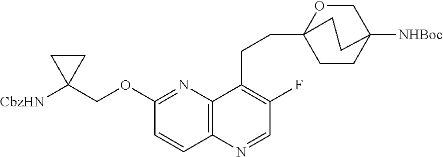 [SMILES: CC(C)(C)OC(=O)NC12CCC(CCc3c(F)cnc4ccc(OCC5(NC(=O)OCc6ccccc6)CC5)nc34)(CC1)OC2]

The title compound (131 mg) was prepared from tert-butyl 1-(2-(3-fluoro-6-hydroxy-1,5-naphthyridin-4-yl)ethyl)-2-oxabicyclo[2.2.2]octan-4-ylcarbamate (100 mg) and benzyl 1-(bromomethyl)cyclopropylcarbamate (81.7 mg) in the same manner as described for Step 1 of EXAMPLE 32.

¹H NMR (CDCl₃) δ 0.98-0.99 (m, 4H), 1.48 (s, 9H), 1.62-1.74 (m, 4H), 1.78-1.92 (m, 2H), 1.94-2.17 (m, 4H), 3.05-3.19 (m, 2H), 3.96 (s, 2H), 4.28 (br, 1H), 4.59 (s, 2H), 4.97 (s, 2H), 5.72 (br, 1H), 7.06 (s, J=8.6 Hz, 1H), 7.19-7.26 (m, 5H), 8.15 (d, J=9.2 Hz, 1H), 8.58 (s, 1H).

MS (ESI⁺) m/z: 621 (MH⁺).

HRMS (ESI⁺) for C₃₄H₄₂FN₄O₆ (MH⁺): calcd, 621.30884. found, 621.30859.

### Step 3

### Preparation of Benzyl 1-((8-(2-(4-Amino-2-oxabicyclo[2.2.2]octan-1-yl)ethyl)-7-fluoro-1,5-naphthyridin-2-yloxy)methyl)cyclopropylcarbamate

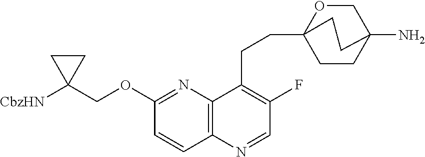 [SMILES: NC12CCC(CCc3c(F)cnc4ccc(OCC5(NC(=O)OCc6ccccc6)CC5)nc34)(CC1)OC2]

The title compound (94.6 mg) was prepared from tert-butyl 1-(2-(3-fluoro-6-((1-benzyloxycarbonylaminocyclopropyl)methoxy)-1,5-naphthyridin-4-yl)ethyl)-2-oxabicyclo[2.2.2]octan-4-ylcarbamate (125 mg) in the same manner as described for Step 2 of EXAMPLE 1.

¹H NMR (CDCl₃) δ 0.98-1.00 (m, 4H), 1.65-1.81 (m, 8H), 1.85-2.05 (m, 2H), 3.08-3.21 (m, 2H), 3.64 (s, 2H), 4.60 (s, 2H), 4.98 (s, 2H), 5.73 (br, 1H), 7.06 (d, J=9.2 Hz, 1H), 7.14-7.31 (m, 5H), 8.16 (d, J=9.2 Hz, 1H), 8.58 (s, 1H).

MS (ESI⁺) m/z: 521 (MH⁺).

HRMS (ESI⁺) for C₂₉H₃₄FN₄O₄ (MH⁺): calcd, 521.25641. found, 521.25602.

### Step 4

### Preparation of Benzyl 1-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropylcarbamate

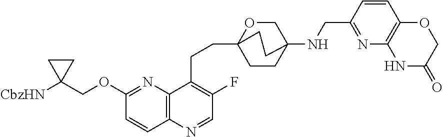 [SMILES: O=C1COc2ccc(CNC34CCC(CCc5c(F)cnc6ccc(OCC7(NC(=O)OCc8ccccc8)CC7)nc56)(CC3)OC4)nc2N1]

The title compound (79.8 mg) was prepared from benzyl 1-((8-(2-(4-amino-2-oxabicyclo[2.2.2]octan-1-yl)ethyl)-7-fluoro-1,5-naphthyridin-2-yloxy)methyl)cyclopropylcarbamate (90.0 mg) and 3-oxo-3,4-dihydro-2H-pyrido[3,2-b][1,4]oxazine-6-carbaldehyde (32.3 mg) in the same manner as described for Step 3 of EXAMPLE 1.

¹H NMR (CDCl₃) δ 0.95-1.05 (m, 4H), 1.59-1.90 (m, 8H), 1.93-2.07 (m, 2H), 3.09-3.20 (m, 2H), 3.75 (s, 2H), 3.77 (s, 2H), 4.59 (s, 2H), 4.63 (s, 2H), 4.99 (s, 2H), 5.70 (br, 1H), 6.94 (d, J=7.9 Hz, 1H), 7.07 (d, J=7.9 Hz, 1H), 7.20 (d, J=7.9 Hz, 1H), 7.26 (br, 5H), 8.16 (d, J=9.2 Hz, 1H), 8.59 (s, 1H).

MS (ESI⁺) m/z: 683 (MH⁺).

HRMS (ESI⁺) for C₃₆H₄₀FN₆O₆ (MH⁺): calcd, 683.29933. found, 683.29934.

### Example 219

The following compound was prepared consistent with the methods described herein.

### 6-((1-(2-(7-Methoxy-4-oxoquinolin-1(4H)-yl)ethyl)-2-oxabicyclo[2.2.2]octan-4-ylamino)methyl)-2H-pyrido[3,2-b][1,4]oxazin-3(4H)-one

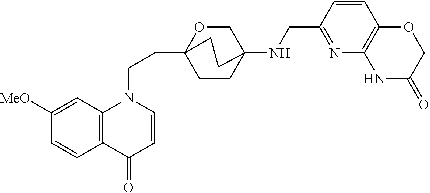 [SMILES: COc1ccc2c(=O)ccn(CCC34CCC(NCc5ccc6c(n5)NC(=O)CO6)(CC3)CO4)c2c1]

¹H NMR (DMSO-d₆) δ 1.61-1.97 (m, 11H), 3.62 (s, 2H), 3.88 (s, 3H), 4.18-4.23 (m, 2H), 4.59 (s, 2H), 5.93 (d, J=7.3 Hz, 1H), 6.95-7.02 (m, 3H), 7.28 (d, J=8.6 Hz, 1H), 7.88 (d, J=7.9 Hz, 1H), 8.37 (d, J=8.6 Hz, 1H), 11.15 (s, 1H).

MS (ESI⁺) m/z: 491 (MH⁺).

HRMS (ESI⁺) for C₂₇H₃₁N₄O₅ (MH⁺): calcd, 491.22944. found, 491.22963.

### Preparation of Intermediates

### Step 1

### Preparation of tert-Butyl 1-(2-(7-Methoxy-4-oxoquinolin-1(4H)-yl)ethyl)-2-oxabicyclo[2.2.2]octan-4-ylcarbamate

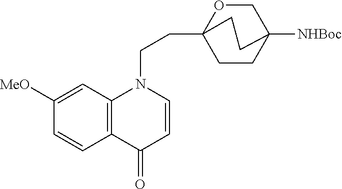 [SMILES: COc1ccc2c(=O)ccn(CCC34CCC(NC(=O)OC(C)(C)C)(CC3)CO4)c2c1]

¹H NMR (CDCl₃) δ 1.45 (s, 9H), 1.65-1.72 (m, 2H), 1.80-1.99 (m, 6H), 2.11-2.17 (m, 2H), 3.92 (s, 3H), 4.02 (s, 2H), 4.10-4.17 (m, 2H), 4.32 (br, 1H), 6.20 (d, J=7.9 Hz, 1H), 6.94-6.98 (m, 2H), 7.46 (d, J=7.9 Hz, 1H), 8.36 (d, J=8.6 Hz, 1H).

MS (ESI⁺) m/z: 429 (MH⁺).

HRMS (ESI⁺) for C₂₄H₃₃N₂O₅ (MH⁺): calcd, 429.23895. found, 429.23878.

### Step 2

### Preparation of 1-(2-(4-Amino-2-oxabicyclo[2.2.2]octan-1-yl)ethyl)-7-methoxyquinolin-4(1H)-one

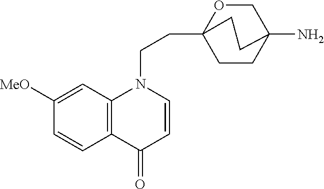 [SMILES: COc1ccc2c(=O)ccn(CCC34CCC(N)(CC3)CO4)c2c1]

¹H NMR (CDCl₃) δ 1.65-1.75 (m, 6H), 1.87-1.96 (m, 4H), 3.66 (s, 2H), 3.93 (s, 3H), 4.14-4.18 (m, 2H), 6.20 (d, J=7.3 Hz, 1H), 6.94-6.98 (m, 2H), 7.46 (d, J=7.3 Hz, 1H), 8.37 (d, J=8.6 Hz, 1H).

MS (EI⁺) m/z: 328 (M⁺).

HRMS (EI⁺) for C₁₉H₂₄N₂O₃ (M⁺): calcd, 328.1787. found, 328.1818.

### Example 220

### 1-(2-(4-((2,3-Dihydro-[1,4]dioxino[2,3-c]pyridin-7-yl)methylamino)-2-oxabicyclo[2.2.2]octan-1-yl)-2-hydroxyethyl)-7-methoxy-1,5-naphthyridin-2(1H)-one (Enantiomer A)

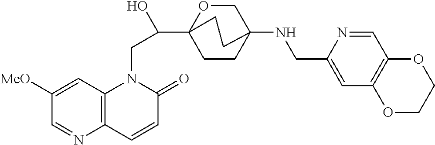 [SMILES: COc1cnc2ccc(=O)n(CC(O)C34CCC(NCc5cc6c(cn5)OCCO6)(CC3)CO4)c2c1]

¹H NMR (CDCl₃) δ 1.77-2.14 (m, 8H), 3.75 (t, J=4.3 Hz, 1H), 3.75 (s, 2H), 3.82 (dd, J=7.9, 3.1 Hz, 1H), 3.86 (dd, J=7.9, 2.4 Hz, 1H), 3.95 (s, 3H), 4.05 (s, 1H), 4.26-4.28 (m, 2H), 4.31-4.34 (m, 2H), 4.43-4.45 (m, 2H), 6.78 (d, J=9.8 Hz, 1H), 6.83 (s, 1H), 7.57 (d, J=2.4 Hz, 1H), 7.91 (d, J=9.2 Hz, 1H), 8.10 (s, 1H), 8.30 (d, J=2.4 Hz, 1H).

MS (ESI) m/z: 495 (MH⁺).

HRMS (ESI) for C₂₆H₃₁N₄O₆ (MH⁺): calcd, 495.22436. found, 495.22468.

### Example 221

### 4-(2-(4-((2,3-Dihydro-[1,4]dioxino[2,3-c]pyridin-7-yl)methylamino)-2-oxabicyclo[2.2.2]octan-1-yl)-2-hydroxyethyl)-6-methoxypyrido[3,2-b]pyrazin-3(4H)-one (Enantiomer A)

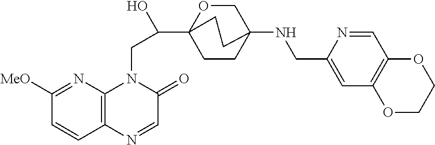 [SMILES: COc1ccc2ncc(=O)n(CC(O)C34CCC(NCc5cc6c(cn5)OCCO6)(CC3)CO4)c2n1]

¹H NMR (CDCl₃) δ 1.76-1.92 (m, 6H), 2.04-2.19 (m, 2H), 3.03 (br, 1H), 3.74 (s, 2H), 3.78 (s, 2H), 3.81 (d, J=12.2 Hz, 1H), 4.03 (s, 3H), 4.26-4.28 (m, 2H), 4.31-4.34 (m, 2H), 4.62 (dd, J=13.4, 9.8 Hz, 1H), 4.73 (dd, J=13.5, 2.4 Hz, 1H), 6.74 (d, J=8.6 Hz, 1H), 6.82 (s, 1H), 8.03 (d, J=8.6 Hz, 1H), 8.09 (s, 1H), 8.19 (s, 1H).

MS (ESI) m/z: 496 (MH⁺).

HRMS (ESI⁺) for C₂₅H₃₀N₅O₆ (MH⁺): calcd, 496.21961. found, 496.21940.

### Example 222

### 4-(2-(4-((2,3-Dihydro-[1,4]dioxino[2,3-c]pyridin-7-yl)methylamino)-2-oxabicyclo[2.2.2]octan-1-yl)-2-hydroxyethyl)-6-methoxypyrido[3,2-b]pyrazin-3(4H)-one (Enantiomer B)

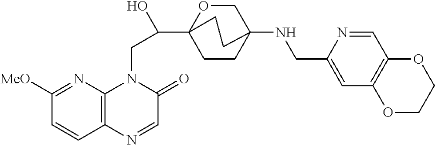 [SMILES: COc1ccc2ncc(=O)n(CC(O)C34CCC(NCc5cc6c(cn5)OCCO6)(CC3)CO4)c2n1]

¹H NMR (CDCl₃) δ 1.76-1.92 (m, 6H), 2.04-2.19 (m, 2H), 3.04 (d, J=6.7 Hz, 1H), 3.74 (s, 2H), 3.78 (s, 2H), 3.83 (ddd, J=9.8, 6.7, 2.4 Hz, 1H), 4.03 (s, 3H), 4.26-4.28 (m, 2H), 4.31-4.34 (m, 2H), 4.62 (dd, J=13.4, 9.8 Hz, 1H), 4.73 (dd, J=13.4, 2.4 Hz, 1H), 6.74 (d, J=8.6 Hz, 1H), 6.82 (s, 1H), 8.03 (d, J=8.6 Hz, 1H), 8.09 (s, 1H), 8.19 (s, 1H).

MS (ESI) m/z: 496 (MH⁺).

HRMS (ESI) for C₂₅H₃₀N₅O₆ (MH⁺): calcd, 496.21961. found, 496.21971.

### Example 223

The following compound was prepared consistent with the methods described herein.

### 6-((1-(2-(6-((3RS,4 SR)-4-Aminotetrahydro furan-3-yloxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

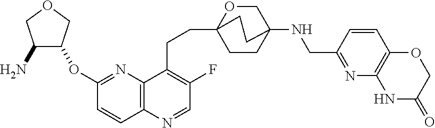

The title compound (16.5 mg) was prepared from benzyl (3SR,4RS)-4-(7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)tetrahydrofuran-3-ylcarbamate (42.0 mg) in the same manner as described for Step 4 of EXAMPLE 38.

¹H NMR (DMSO-d₆) δ 1.49-1.76 (m, 8H), 1.77-2.14 (m, 5H), 3.03-3.17 (m, 2H), 3.46 (dd, J=8.6, 3.1 Hz, 1H), 3.53-3.68 (m, 5H), 3.84 (d, J=9.2 Hz, 1H), 3.98 (dd, J=8.6, 5.5 Hz, 1H), 4.21 (dd, J=10.4, 4.9 Hz, 1H), 4.59 (s, 2H), 5.23 (d, J=4.3 Hz, 1H), 7.01 (d, J=7.9 Hz, 1H), 7.22 (d, J=9.2 Hz, 1H), 7.28 (d, J=7.9 Hz, 1H), 8.27 (d, J=8.6 Hz, 1H), 8.75 (s, 1H), 11.16 (s, 1H).

MS (ESI⁺) m/z: 565 (MH⁺).

HRMS (ESI) for C₂₉H₃₄FN₆O₅ (MH⁺): calcd, 565.25747. found, 565.25705.

### Preparation of Intermediates

### Step 1

### Preparation of Benzyl (3R,4R)-4-(tert-Butyldimethylsilyloxy)tetrahydrofuran-3-ylcarbamate

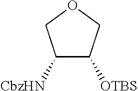 [SMILES: CC(C)(C)[Si](C)(C)O[C@H]1COC[C@H]1NC(=O)OCc1ccccc1]

The title compound (252 mg) was prepared from (3R,4R)-4-(tert-butyldimethylsilyloxy)tetrahydrofuran-3-amine (200 mg) in the same manner as described for Intermediate X.2.

¹H NMR (CDCl₃) δ 0.07 (s, 6H), 0.90 (s, 9H), 3.54 (t, J=7.9 Hz, 1H), 3.65 (dd, J=9.8, 3.1 Hz, 1H), 4.00 (dd, J=9.8, 4.9 Hz, 1H), 4.03 (t, J=8.6 Hz, 1H), 4.18-4.21 (m, 1H), 4.27-4.34 (m, 1H), 5.11 (s, 2H), 5.19 (d, J=7.3 Hz, 1H), 7.29-7.38 (m, 5H).

MS (CI⁺) m/z: 352 (MH⁺).

HRMS (CI⁺) for C₁₈H₃₀NO₄Si (MH⁺): calcd, 352.1944. found, 352.1909.

### Step 2

### Preparation of Benzyl (3R,4R)-4-Hydroxytetrahydrofuran-3-ylcarbamate

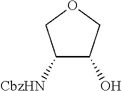 [SMILES: O=C(N[C@@H]1COC[C@@H]1O)OCc1ccccc1]

To a solution of benzyl (3R,4R)-4-(tert-butyldimethylsilyloxy)tetrahydrofuran-3-ylcarbamate (240 mg) in tetrahydrofuran was added a solution of tetrabutylammonium fluoride (1 M, 0.75 mL), the mixture was stirred under cooling with ice bath for 2 hours. The mixture was diluted with ethyl acetate, the mixture was washed with water. The organic extracts were dried over anhydrous sodium sulfate, filtered, and then concentrated in vacuo. Flash chromatography (silica, hexane:ethyl acetate=1:3) of the residue gave the title compound (136 mg).

¹H NMR (CDCl₃) δ 2.10 (br, 1H), 3.56 (t, J=7.9 Hz, 1H), 3.79 (dd, J=10.3, 2.4 Hz, 1H), 4.01 (dd, J=10.3, 4.3 Hz, 1H), 4.06 (t, J=7.9 Hz, 1H), 4.20-4.28 (m, 1H), 4.37-4.41 (m, 1H), 5.12 (s, 2H), 5.30 (br, 1H), 7.29-7.44 (m, 5H).

MS (CI⁺) m/z: 238 (MH⁺).

HRMS (CI⁺) for C₁₂H₁₆NO₄ (MH⁺): calcd, 238.1079. found, 238.1070.

### Step 3

### Preparation of Benzyl (3R,4S)-4-Bromotetrahydrofuran-3-ylcarbamate

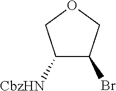 [SMILES: O=C(N[C@@H]1COC[C@H]1Br)OCc1ccccc1]

The title compound (66.2 mg) was prepared from benzyl (3R,4R)-4-hydroxytetrahydrofuran-3-ylcarbamate (130 mg) in the same manner as described for X.

¹H NMR (CDCl₃) δ 3.74 (dd, J=9.8, 1.8 Hz, 1H), 4.07 (dd, J=11.0, 3.1 Hz, 1H), 4.21-4.31 (m, 2H), 4.35 (dd, J=11.0, 5.5 Hz, 1H), 4.43 (br, 1H), 4.99-5.20 (m, 3H), 7.30-7.42 (m, 5H).

MS (CI⁺) m/z: 300 (MH⁺).

HRMS (CI⁺) for C₁₂H₁₅BrNO₃ (MH⁺): calcd, 300.0235. found, 300.0251.

### Step 4

### Preparation of tert-Butyl 1-(2-(6-((3SR,4RS)-4-Benzyloxycarbonyl-3-fluoro-tetrahydro furan-3-yloxy)-1,5-naphthyridin-4-yl)ethyl)-2-oxabicyclo[2.2.2]octan-4-ylcarbamate

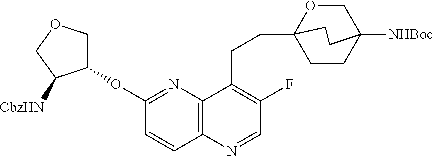 [SMILES: CC(C)(C)OC(=O)NC12CCC(CCc3c(F)cnc4ccc(O[C@H]5COC[C@@H]5NC(=O)OCc5ccccc5)nc34)(CC1)OC2]

The title compound (58.6 mg) was prepared from tert-butyl 1-(2-(3-fluoro-6-hydroxy-1,5-naphthyridin-4-yl)ethyl)-2-oxabicyclo[2.2.2]octan-4-ylcarbamate (79.7 mg) and benzyl (3R,4S)-4-bromotetrahydrofuran-3-ylcarbamate (63.0 mg) in the same manner as described for Step 1 of EXAMPLE 32.

¹H NMR (CDCl₃) δ 1.44 (s, 9H), 1.55-1.99 (m, 9H), 2.05-2.23 (m, 1H), 2.86-3.02 (m, 1H), 3.18-3.30 (m, 1H), 3.72-3.82 (m, 1H), 3.86-3.93 (m, 1H), 3.95-4.01 (m, 1H), 4.04 (dd, J=10.4, 3.1 Hz, 1H), 4.16-4.22 (m, 2H), 4.25 (dd, J=10.4, 6.1 Hz, 1H), 4.28-4.37 (m, 1H), 5.08 (dd, 15.9, 12.2 Hz, 2H), 5.61-5.68 (m, 1H), 6.64 (br, 1H), 7.08 (d, J=9.2 Hz, 1H), 7.28-7.38 (m, 5H), 8.20 (d, J=9.2 Hz, 1H), 8.61 (s, 1H).

MS (ESI⁺) m/z: 637 (MH⁺).

HRMS (ESI⁺) for C₃₄H₄₂FN₄O₇ (MH⁺): calcd, 637.30375. found, 637.30323.

### Step 5

### Preparation of benzyl (3SR,4RS)-4-(8-(2-(4-Amino-2-oxabicyclo[2.2.2]octan-1-yl)ethyl)-7-fluoro-1,5-naphthyridin-2-yloxy)tetrahydrofuran-3-ylcarbamate

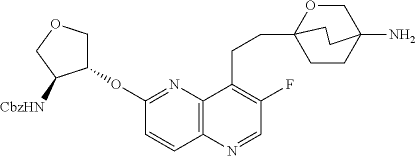 [SMILES: NC12CCC(CCc3c(F)cnc4ccc(O[C@H]5COC[C@@H]5NC(=O)OCc5ccccc5)nc34)(CC1)OC2]

The title compound (44.8 mg) was prepared from tert-butyl 1-(2-(6-((3SR,4RS)-4-benzyloxycarbonyl-3-fluoro-tetrahydrofuran-3-yloxy)-1,5-naphthyridin-4-yl)ethyl)-2-oxabicyclo[2.2.2]octan-4-ylcarbamate (58.0 mg) in the same manner as described for Step 2 of EXAMPLE 32.

¹H NMR (CDCl₃) δ 1.22-2.02 (m, 10H), 2.88-3.02 (m, 1H), 3.21-3.32 (m, 1H), 3.51-3.69 (m, 2H), 3.74-3.84 (m, 1H), 4.04 (dd, J=10.4, 3.1 Hz, 1H), 4.15-4.21 (m, 1H), 4.24 (dd, J=10.4, 6.1 Hz, 1H), 4.28-4.39 (m, 1H), 5.08 (s, 2H), 5.66-5.71 (m, 1H), 7.08 (d, J=8.6 Hz, 1H), 7.28-7.40 (m, 5H), 8.21 (d, J=9.2 Hz, 1H), 8.62 (s, 1H).

MS (ESI⁺) m/z: 537 (MH⁺).

HRMS (ESI⁺) for C₂₉H₃₄FN₄O₅ (MH⁺): calcd, 537.25132. found, 537.25047.

### Step 6

### Preparation of Benzyl (3SR,4RS)-4-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)tetrahydrofuran-3-ylcarbamate

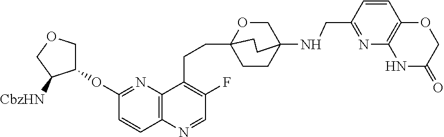 [SMILES: O=C1COc2ccc(CNC34CCC(CCc5c(F)cnc6ccc(O[C@H]7COC[C@@H]7NC(=O)OCc7ccccc7)nc56)(CC3)OC4)nc2N1]

The title compound (44.7 mg) was prepared from benzyl (3SR,4RS)-4-(8-(2-(4-amino-2-oxabicyclo[2.2.2]octan-1-yl)ethyl)-7-fluoro-1,5-naphthyridin-2-yloxy)tetrahydrofuran-3-ylcarbamate (44.0 mg) and 3-oxo-3,4-dihydro-2H-pyrido[3,2-b][1,4]oxazine-6-carbaldehyde (15.3 mg) in the same manner as described for Step 3 of EXAMPLE 1.

¹H NMR (CDCl₃) δ 1.54-1.98 (m, 10H), 3.22-3.34 (m, 1H), 3.60-3.83 (m, 5H), 4.02 (dd, J=10.4, 3.1 Hz, 1H), 4.17-4.22 (m, 1H), 4.24 (dd, J=10.4, 6.1 Hz, 1H), 4.28-4.36 (m, 1H), 4.64 (s, 2H), 5.08 (s, 2H), 5.66-5.72 (m, 1H), 6.71 (br, 1H), 6.90 (d, J=7.9 Hz, 1H), 7.09 (d, J=9.2 Hz, 1H), 7.20 (d, J=7.9 Hz, 1H), 7.28-7.38 (m, 5H), 8.21 (d, J=9.2 Hz, 1H), 8.62 (s, 1H).

MS (ESI⁺) m/z: 699 (MH⁺).

HRMS (ESI⁺) for C₃₇H₄₀FN₆O₇ (MH⁺): calcd, 699.29425. found, 699.29350.

### Example 224

The following compound was prepared consistent with the methods described herein.

### 6-((1-(2-(6-((3S,4R)-4-Aminotetrahydrofuran-3-yloxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

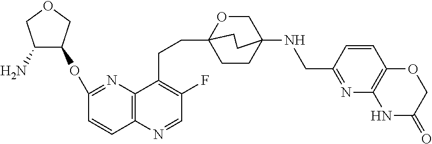 [SMILES: N[C@@H]1COC[C@H]1Oc1ccc2ncc(F)c(CCC34CCC(NCc5ccc6c(n5)NC(=O)CO6)(CC3)CO4)c2n1]

The title compound (9.6 mg) was prepared from benzyl (3R,4S)-4-(7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)tetrahydrofuran-3-ylcarbamate (26.0 mg) in the same manner as described for Step 4 of EXAMPLE 38.

¹H NMR (DMSO-d₆) δ 1.55-1.77 (m, 8H), 1.78-2.03 (m, 4H), 3.05-3.16 (m, 2H), 3.46 (dd, J=9.2, 3.0 Hz, 1H), 3.56-3.66 (m, 5H), 3.84 (d, J=9.8 Hz, 1H), 3.98 (dd, J=8.6, 5.5 Hz, 1H), 4.21 (dd, J=10.4, 4.3 Hz, 1H), 4.59 (s, 2H), 5.23 (d, J=4.3 Hz, 1H), 7.01 (d, J=7.9 Hz, 1H), 7.22 (d, J=9.2 Hz, 1H), 7.28 (d, J=7.9 Hz, 1H), 8.27 (d, J=9.2 Hz, 1H), 8.75 (s, 1H), 11.17 (s, 1H).

MS (ESI⁺) m/z: 565 (MH⁺).

HRMS (ESI⁺) for C₂₉H₃₄FN₆O₅ (MH⁺): calcd, 565.25747. found, 565.25754.

### Preparation of Intermediates

### Step 1

### Preparation of Benzyl (3R,45)-4-Hydroxytetrahydrofuran-3-ylcarbamate

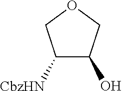 [SMILES: O=C(N[C@@H]1COC[C@H]1O)OCc1ccccc1]

A mixture of (3S,4R)-4-azidotetrahydrofuran-3-ol (300 mg), Lindlar catalyst (45.0 mg) and methanol was stirred at room temperature for 3 hours under H₂ atmosphere (1 kg/cm²). After the insoluble materials were filtered off, the filtrate was concentrated in vacuo. Flash chromatography (silica, hexane:ethyl acetate=1:2) of the residue gave the title compound (412 mg).

¹H NMR (CDCl₃) δ 2.94 (d, J=2.4 Hz, 1H), 3.66 (dd, J=16.5, 2.4 Hz, 1H), 3.68 (dd, J=17.1, 3.1 Hz, 1H), 3.97-4.12 (m, 3H), 4.32 (br, 1H), 4.97 (br, 1H), 5.11 (dd, J=15.3, 12.2 Hz, 2H), 7.29-7.41 (m, 5H).

MS (CI⁺) m/z: 238 (MH⁺).

HRMS (CI⁺) for C₁₂H₁₆NO₄ (MH⁺): calcd, 238.1079. found, 238.1101.

### Step 2

### Preparation of Benzyl (3R,4R)-4-Bromotetrahydrofuran-3-ylcarbamate

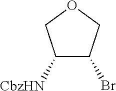 [SMILES: O=C(N[C@@H]1COC[C@@H]1Br)OCc1ccccc1]

The title compound (141 mg) was prepared from benzyl (3R,4S)-4-hydroxytetrahydrofuran-3-ylcarbamate (400 mg) in the same manner as described for Intermediate X.

¹H NMR (CDCl₃) δ 3.63 (t, J=8.6 Hz, 1H), 4.03-4.16 (m, 1H), 4.22 (dd, J=11.0, 2.4 Hz), 4.41 (dd, J=11.0, 4.3 Hz, 1H), 4.36-4.48 (m, 1H), 4.60 (br, 1H), 5.06-5.18 (m, 3H), 7.31-7.43 (m, 5H).

MS (CI⁺) m/z: 300 (MH⁺).

HRMS (CI⁺) for C₁₂H₁₅BrNO₃ (MH⁺): calcd, 300.0235. found, 300.0247.

### Step 3

### Preparation of tert-Butyl 1-(2-(6-((3R,45)-4-Benzyloxycarbonyl-3-fluoro-tetrahydro furan-3-yloxy)-1,5-naphthyridin-4-yl)ethyl)-2-oxabicyclo[2.2.2]octan-4-ylcarbamate

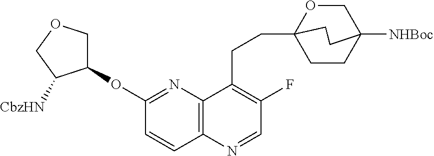 [SMILES: CC(C)(C)OC(=O)NC12CCC(CCc3c(F)cnc4ccc(O[C@@H]5COC[C@H]5NC(=O)OCc5ccccc5)nc34)(CC1)OC2]

The title compound (55.8 mg) was prepared from tert-butyl 1-(2-(3-fluoro-6-hydroxy-1,5-naphthyridin-4-yl)ethyl)-2-oxabicyclo[2.2.2]octan-4-ylcarbamate (100 mg) and benzyl (3R,4R)-4-bromotetrahydrofuran-3-ylcarbamate (79.1 mg) in the same manner as described for Step 1 of EXAMPLE 32.

¹H NMR (CDCl₃) δ 1.44 (s, 9H), 1.55-1.99 (m, 10H), 2.96-3.00 (m, 1H), 3.18-3.30 (m, 1H), 3.73-3.81 (m, 1H), 3.86-3.93 (m, 1H), 3.95-4.07 (m, 1H), 4.04 (dd, J=11.0, 3.1 Hz, 1H), 4.16-4.23 (m, 2H), 4.25 (dd, J=10.4, 5.5 Hz, 1H), 4.28-4.35 (m, 1H), 5.08 (dd, J=15.3, 12.2 Hz, 2H), 5.65, (q, J=3.1 Hz, 1H), 6.63 (br, 1H), 7.08 (d, J=9.2 Hz, 1H), 7.30-7.40 (m, 5H), 8.20 (d, J=9.2 Hz, 1H), 8.61 (s, 1H).

MS (ESI⁺) m/z: 637 (MH⁺).

HRMS (ESI⁺) for C₃₄H₄₂FN₄O₇ (MH⁺): calcd, 637.30375. found, 637.30315.

### Step 4

### Preparation of Benzyl (3R,4S)-4-(8-(2-(4-Amino-2-oxabicyclo[2.2.2]octan-1-yl)ethyl)-7-fluoro-1,5-naphthyridin-2-yloxy)tetrahydrofuran-3-ylcarbamate

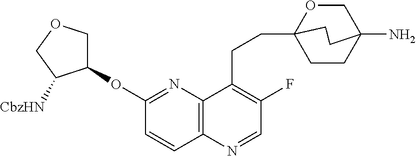 [SMILES: NC12CCC(CCc3c(F)cnc4ccc(O[C@@H]5COC[C@H]5NC(=O)OCc5ccccc5)nc34)(CC1)OC2]

The title compound (39.2 mg) was prepared from tert-butyl 1-(2-(6-((3R,45)-4-benzyloxycarbonyl-3-fluoro-tetrahydrofuran-3-yloxy)-1,5-naphthyridin-4-yl)ethyl)-2-oxabicyclo[2.2.2]octan-4-ylcarbamate (54.0 mg) in the same manner as described for Step 2 of EXAMPLE 32.

¹H NMR (CDCl₃) δ 1.28-1.98 (m, 10H), 2.87-3.01 (m, 1H), 3.22-3.30 (m, 1H), 3.51-3.68 (m, 2H), 3.73-3.84 (m, 1H), 3.98-4.08 (m, 1H), 4.13-4.21 (m, 1H), 4.24 (dd, J=10.4, 5.5 Hz, 1H), 4.28-4.37 (m, 1H), 5.08 (s, 2H), 5.64-5.72 (m, 1H), 7.08 (d, J=8.6 Hz, 1H), 7.27-7.41 (m, 5H), 8.21 (d, J=8.6 Hz, 1H), 8.62 (s, 1H).

MS (ESI⁺) m/z: 537 (MH⁺).

HRMS (ESI⁺) for C₂₉H₃₄FN₄O₅ (MH⁺): calcd, 537.25132. found, 537.25171.

### Step 5

### Preparation of Benzyl (3R,4S)-4-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)tetrahydrofuran-3-ylcarbamate

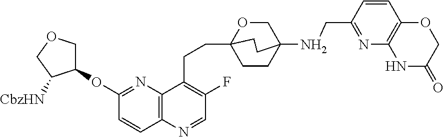 [SMILES: O=C1COc2ccc(CNC34CCC(CCc5c(F)cnc6ccc(O[C@@H]7COC[C@H]7NC(=O)OCc7ccccc7)nc56)(CC3)OC4)nc2N1]

The title compound (30.4 mg) was prepared from benzyl (3R,4S)-4-(8-(2-(4-amino-2-oxabicyclo[2.2.2]octan-1-yl)ethyl)-7-fluoro-1,5-naphthyridin-2-yloxy)tetrahydrofuran-3-ylcarbamate (35.0 mg) and 3-oxo-3,4-dihydro-2H-pyrido[3,2-b][1,4]oxazine-6-carbaldehyde (12.2 mg) in the same manner as described for Step 3 of EXAMPLE 1.

¹H NMR (CDCl₃) δ 1.62-2.05 (m, 10H), 2.86-3.06 (m, 1H), 3.22-3.35 (m, 1H), 3.59-3.88 (m, 2H), 3.97-4.08 (m, 1H), 4.16-4.36 (m, 3H), 4.64 (s, 2H), 5.08 (s, 2H), 5.64-5.77 (m, 1H), 6.72 (s, 1H), 6.91 (d, J=8.6 Hz, 1H), 7.09 (d, J=9.2 Hz, 1H), 7.20 (d, J=8.0 Hz, 1H), 7.28-7.38 (m, 5H), 8.21 (d, J=9.2 Hz, 1H), 8.62 (s, 1H).

MS (ESI⁺) m/z: 699 (MH⁺).

HRMS (ESI⁺) for C₃₇H₄₀FN₆O₇ (MH⁺): calcd, 699.29425. found, 699.29502.

### Example 225

### HCl Salt of Example 158

### 6-((1-(2-(3-Fluoro-6-(3-hydroxypropoxy)-1,5-nap hthyridin-4-yl)-1-hydroxyethyl)-2-oxabicyclo[2.2.2]octan-4-ylamino)methyl)-2H-pyrido[3,2-b][1,4]oxazin-3(4H)-one Hydrochloride (Enantiomer A)

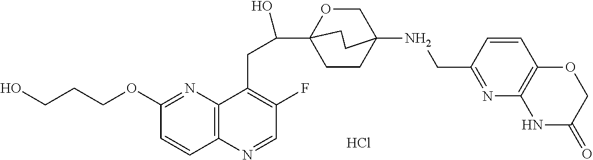 [SMILES: O=C1COc2ccc(CNC34CCC(C(O)Cc5c(F)cnc6ccc(OCCCO)nc56)(CC3)OC4)nc2N1 ; Cl]

¹H NMR (DMSO-d₆) δ 1.82-2.14 (m, 10H), 3.01 (dd, J=12.2, 10.4 Hz, 1H), 3.58 (t, J=6.1 Hz, 2H), 3.75-3.83 (m, 1H), 3.90 (s, 2H), 4.11 (t, J=6.1 Hz, 2H), 4.53 (t, J=6.1 Hz, 1H), 4.59 (brs, 1H), 4.69 (s, 3H), 7.20 (d, J=9.2 Hz, 1H), 7.20 (d, J=7.9 Hz, 1H), 7.46 (d, J=7.9 Hz, 1H), 8.26 (d, J=9.2 Hz, 1H), 8.73 (s, 1H), 9.23 (s, 2H), 11.33 (s, 1H).

MS (ESI⁺) m/z: 554 (MH⁺) (as free base).

HRMS (ESI⁺) for C₂₈H₃₃FN₅O₆ (MH⁺) (as free base): calcd, 554.24149. found, 554.24162.

### Example 226

### 6-((1-(2-(3-Fluoro-6-((5-hydroxy-4-oxo-1,4-dihydropyridin-2-yl)methoxy)-1,5-naphthyridin-4-yl)ethyl)-2-oxabicyclo[2.2.2]octan-4-ylamino)methyl)-2H-pyrido[3,2-b][1,4]oxazin-3(4H)-one

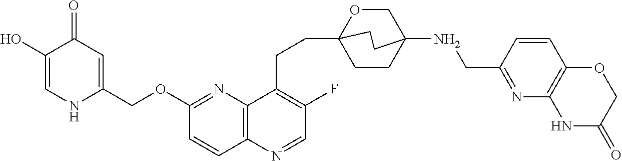 [SMILES: O=C1COc2ccc(CNC34CCC(CCc5c(F)cnc6ccc(OCc7cc(=O)c(O)c[nH]7)nc56)(CC3)OC4)nc2N1]

¹H NMR (DMSO-d₆) δ 1.51-1.72 (m, 8H), 1.77-1.91 (m, 2H), 3.03-3.12 (m, 2H), 3.57 (s, 2H), 3.62 (s, 2H), 4.58 (s, 2H), 5.40 (s, 2H), 6.47 (brs, 1H), 7.00 (d, J=7.9 Hz, 1H), 7.27 (d, J=7.9 Hz, 1H), 7.31 (d, J=8.6 Hz, 1H), 7.50 (brs, 1H), 8.32 (d, J=9.2 Hz, 1H), 8.76 (s, 1H).

MS (ESI⁺) m/z: 603 (MH⁺).

HRMS (ESI⁺) for C₃₁H₃₂FN₆O₆ (MH⁺): calcd, 603.23673. found, 603.23577.

### Example 227

### Methyl 247-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylate (Enantiomer A)

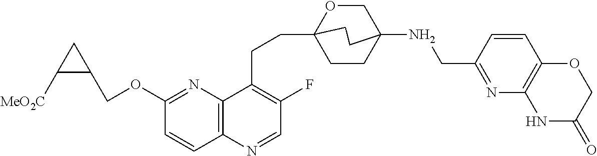 [SMILES: COC(=O)C1CC1COc1ccc2ncc(F)c(CCC34CCC(NCc5ccc6c(n5)NC(=O)CO6)(CC3)CO4)c2n1]

¹H NMR (CDCl₃) δ 1.19-1.28 (m, 2H), 1.72-2.08 (m, 12H), 3.12-3.24 (m, 2H), 3.69 (s, 3H), 3.76 (s, 2H), 3.77 (s, 2H), 4.48 (dd, J=11.6, 8.0 Hz, 1H), 4.63 (s, 2H), 4.90 (dd, J=11.6, 6.7 Hz, 1H), 6.94 (d, J=7.9 Hz, 1H), 7.04 (d, J=8.6 Hz, 1H), 7.20 (d, J=7.9 Hz, 1H), 8.16 (d, J=9.2 Hz, 1H), 8.16 (br, 1H), 8.59 (s, 1H).

MS (ESI⁺) m/z: 592 (MH⁺).

HRMS (ESI⁺) for C₃₁H₃₅FN₅O₆ (MH⁺): calcd, 592.25714. found, 592.25743.

### Preparation of Intermediates

### Step 1

### Preparation of (1RS,2SR)-Methyl 2-((8-(2-(4-(tert-Butoxycarbonylamino)-2-oxabicyclo[2.2.2]octan-1-yl)ethyl)-7-fluoro-1,5-naphthyridin-2-yloxy)methyl)cyclopropanecarboxylate

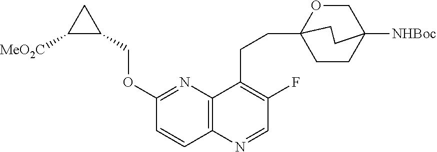 [SMILES: COC(=O)[C@@H]1C[C@@H]1COc1ccc2ncc(F)c(CCC34CCC(NC(=O)OC(C)(C)C)(CC3)CO4)c2n1]

¹H NMR (CDCl₃) δ 1.19-1.30 (m, 2H), 1.44 (s, 9H), 1.70-2.17 (m, 12H), 3.10-3.24 (m, 2H), 3.69 (s, 3H), 3.96 (s, 2H), 4.28 (s, 1H), 4.47 (dd, J=11.6, 8.6 Hz, 1H), 4.90 (dd, J=11.6, 6.1 Hz, 1H), 7.03 (d, J=9.2 Hz, 1H), 8.15 (d, J=9.2 Hz, 1H), 8.58 (s, 1H).

MS (ESI⁺) m/z: 530 (MH⁺).

HRMS (ESI⁺) for C₂₈H₃₇FN₃O₆ (MH⁺): calcd, 530.26664. found, 530.26634.

### Step 2

### Preparation of (1RS,2SR)-Methyl 2-((8-(2-(4-Amino-2-oxabicyclo[2.2.2]octan-1-yl)ethyl)-7-fluoro-1,5-naphthyridin-2-yloxy)methyl)cyclopropanecarboxylate

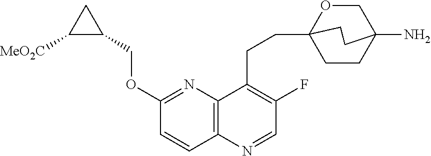 [SMILES: COC(=O)[C@@H]1C[C@@H]1COc1ccc2ncc(F)c(CCC34CCC(N)(CC3)CO4)c2n1]

¹H NMR (CDCl₃) δ 1.19-1.28 (m, 2H), 1.63-2.06 (m, 12H), 3.11-3.24 (m, 2H), 3.65 (s, 2H), 3.69 (s, 3H), 4.48 (dd, J=11.6, 8.6 Hz, 1H), 4.89 (dd, J=11.6, 6.1 Hz, 1H), 7.04 (d, J=9.2 Hz, 1H), 8.15 (d, J=9.2 Hz, 1H), 8.58 (s, 1H).

MS (ESI⁺) m/z: 430 (MH⁺).

HRMS (ESI⁺) for C₂₃H₂₉FN₃O₄ (MH⁺): calcd, 430.21421. found, 430.21399.

### Example 228

The following compound was prepared consistent with the methods described herein.

### Methyl 2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylate (Enantiomer B)

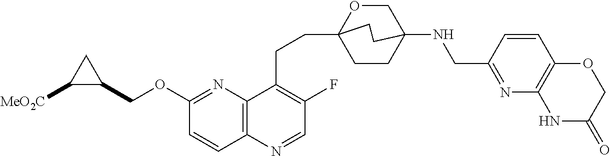 [SMILES: COC(=O)[C@H]1C[C@H]1COc1ccc2ncc(F)c(CCC34CCC(NCc5ccc6c(n5)NC(=O)CO6)(CC3)CO4)c2n1]

¹H NMR (CDCl₃) δ 1.19-1.28 (m, 2H), 1.72-2.08 (m, 12H), 3.12-3.24 (m, 2H), 3.69 (s, 3H), 3.76 (s, 2H), 3.78 (s, 2H), 4.48 (dd, J=11.6, 8.6 Hz, 1H), 4.63 (s, 2H), 4.90 (dd, J=11.6, 6.1 Hz, 1H), 6.94 (d, J=7.9 Hz, 1H), 7.04 (d, J=8.6 Hz, 1H), 7.20 (d, J=7.9 Hz, 1H), 8.16 (d, J=9.2 Hz, 1H), 8.16 (br, 1H), 8.59 (s, 1H).

MS (ESI⁺) m/z: 592 (MH⁺).

HRMS (ESI⁺) for C₃₁H₃₅FN₅O₆ (MH⁺): calcd, 592.25714. found, 592.25642.

### Example 229

The following compound was prepared consistent with the methods described herein.

### 6-((1-(2-(3-Fluoro-6-methoxy-1,5-naphthyridin-4-yl)-2-hydroxyethyl)-2-oxabicyclo[2.2.2]octan-4-ylamino)methyl)-2H-pyrido[3,2-b][1,4]oxazin-3(4H)-one (Enantiomer A)

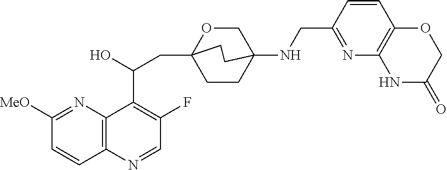 [SMILES: COc1ccc2ncc(F)c(C(O)CC34CCC(NCc5ccc6c(n5)NC(=O)CO6)(CC3)CO4)c2n1]

¹H NMR (DMSO-d₆) δ 1.75-2.04 (m, 8H), 2.13-2.24 (m, 2H), 3.70 (d, J=6.1 Hz, 1H), 3.78 (d, J=6.1 Hz, 1H), 4.05 (brs, 5H), 4.67 (s, 2H), 5.36 (brs, 1H), 5.95 (brs, 1H), 7.19 (d, J=7.9 Hz, 1H), 7.25 (d, J=9.2 Hz, 1H), 7.43 (d, J=7.9 Hz, 1H), 8.29 (d, J=9.2 Hz, 1H), 8.76 (d, J=1.8 Hz, 1H), 9.24 (brs, 2H), 11.29 (s, 1H).

MS (ESI⁺) m/z: 510 (MH⁺) (as free base).

HRMS (ESI⁺) for C₂₆H₂₉FN₅O₅ (MH⁺) (as free base): calcd, 510.21527. found 510.21529.

### Example 230

The following compound was prepared consistent with the methods described herein.

### 6-((1-(2-(3-Fluoro-6-methoxy-1,5-naphthyridin-4-yl)-2-hydroxyethyl)-2-oxabicyclo[2.2.2]octan-4-ylamino)methyl)-2H-pyrido[3,2-b][1,4]oxazin-3(4H)-one (Enantiomer B)

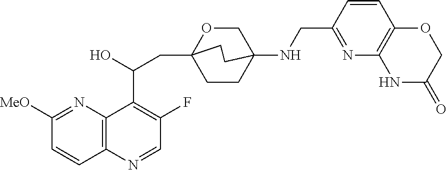 [SMILES: COc1ccc2ncc(F)c(C(O)CC34CCC(NCc5ccc6c(n5)NC(=O)CO6)(CC3)CO4)c2n1]

¹H NMR (DMSO-d₆) δ 1.75-2.03 (m, 8H), 2.13-2.23 (m, 2H), 3.70 (d, J=6.1 Hz, 1H), 3.78 (dd, J=7.9, 2.4 Hz, 1H), 4.05 (brs, 5H), 4.67 (s, 2H), 5.36 (brs, 1H), 5.95 (t, J=6.1 Hz, 1H), 7.19 (d, J=7.9 Hz, 1H), 7.25 (d, J=9.2 Hz, 1H), 7.43 (d, J=7.9 Hz, 1H), 8.29 (d, J=9.2 Hz, 1H), 8.76 (d, J=1.8 Hz, 1H), 9.24 (brs, 2H), 11.29 (s, 1H).

MS (ESI⁺) m/z: 510 (MH⁺) (as free base).

HRMS (ESI⁺) for C₂₆H₂₉FN₅O₅ (MH⁺) (as free base): calcd, 510.21527. found 510.21559.

### Example 231

The following compound was prepared consistent with the methods described herein.

### 3-((1-(2-(3-Fluoro-6-methoxy-1,5-naphthyridin-4-yl)ethyl)-2-oxabicyclo[2.2.2]octan-4-ylamino)methyl)-1-methyl-1,5-naphthyridin-2(1H)-one

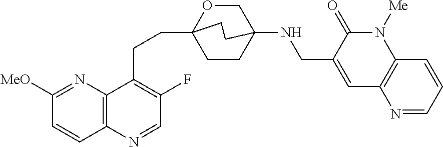 [SMILES: COc1ccc2ncc(F)c(CCC34CCC(NCc5cc6ncccc6n(C)c5=O)(CC3)CO4)c2n1]

¹H NMR (DMSO-d₆) δ 1.60-1.80 (m, 8H), 1.84-1.95 (m, 2H), 2.05-2.15 (m, 1H), 3.07-3.16 (m, 2H), 3.60-3.66 (m, 4H), 3.64 (s, 3H), 4.03 (s, 3H), 7.22 (d, J=9.2 Hz, 1H), 7.56 (dd, J=8.6, 4.9 Hz, 1H), 7.94-7.99 (m, 3H), 8.25 (d, J=9.2 Hz, 1H), 8.52 (dd, J=4.3, 1.2 Hz, 1H), 8.74 (s, 1H).

MS (ESI⁺) m/z: 504 (MH⁺).

HRMS (ESI⁺) for C₂₈H₃₁FN₅O₃ (MH⁺): calcd, 504.24109. found 504.24112.

### Example 232

The following compound was prepared consistent with the methods described herein.

### 2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylic Acid (Enantiomer A)

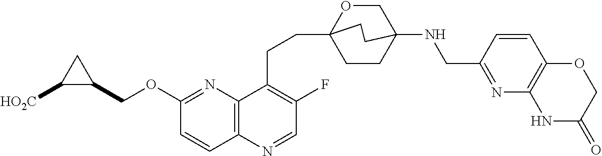 [SMILES: O=C1COc2ccc(CNC34CCC(CCc5c(F)cnc6ccc(OC[C@@H]7C[C@@H]7C(=O)O)nc56)(CC3)OC4)nc2N1]

¹H NMR (DMSO-d₆) δ 0.95-1.02 (m, 1H), 1.12-1.26 (m, 1H), 1.57-1.98 (m, 12H), 3.05-3.13 (m, 2H), 3.58 (s, 2H), 3.63 (s, 2H), 4.49 (dd, J=11.6, 8.6 Hz, 1H), 4.59 (s, 2H), 4.76 (dd, J=11.6, 6.1 Hz, 1H), 7.01 (d, J=7.9 Hz, 1H), 7.20 (d, J=9.2 Hz, 1H), 7.28 (d, J=7.9 Hz, 1H), 8.25 (d, J=9.2 Hz, 1H), 8.73 (s, 1H), 11.15 (s, 1H).

MS (ESI⁺) m/z: 578 (MH⁺).

HRMS (ESI⁺) for C₃₀H₃₃FN₅O₆ (MH⁺): calcd, 578.24149. found, 578.24104.

### Example 233

The following compound was prepared consistent with the methods described herein.

### 2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylic Acid (Enantiomer B)

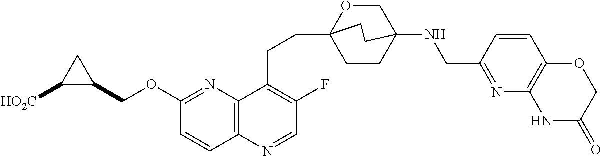 [SMILES: O=C1COc2ccc(CNC34CCC(CCc5c(F)cnc6ccc(OC[C@@H]7C[C@@H]7C(=O)O)nc56)(CC3)OC4)nc2N1]

¹H NMR (DMSO-d₆) δ 0.95-1.02 (m, 1H), 1.13-1.23 (m, 1H), 1.65-2.05 (m, 12H), 3.04-3.14 (m, 2H), 3.50-4.25 (m, 4H), 4.49 (dd, J=11.6, 9.2 Hz, 1H), 4.65 (s, 2H), 4.76 (dd, J=11.6, 6.1 Hz, 1H), 7.11 (br, 1H), 7.21 (d, J=9.2 Hz, 1H), 7.38 (br, 1H), 8.26 (d, J=8.6 Hz, 1H), 8.74 (s, 1H), 11.25 (s, 1H).

MS (ESI⁺) m/z: 578 (MH⁺).

HRMS (ESI⁺) for C₃₀H₃₃FN₅O₆ (MH⁺): calcd, 578.24149. found, 578.24145.

### Example 234

The following compound was prepared consistent with the methods described herein.

### 6-((1-(2-(3-Fluoro-6-hydroxy-1,5-naphthyridin-4-yl)ethyl)-2-oxabicyclo[2.2.2]octan-4-ylamino)methyl)-2H-pyrido[3,2-b][1,4]oxazin-3(4H)-one

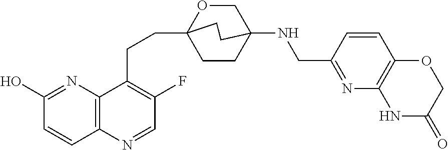 [SMILES: O=C1COc2ccc(CNC34CCC(CCc5c(F)cnc6ccc(O)nc56)(CC3)OC4)nc2N1]

¹H NMR (CDCl₃) δ 1.50-1.88 (m, 10H), 2.85-2.95 (m, 2H), 3.55-3.65 (m, 5H), 4.59 (s, 2H), 6.69 (d, J=9.8 Hz, 1H), 7.01 (d, J=7.9 Hz, 1H), 7.27 (d, J=7.9 Hz, 1H), 7.89 (d, J=9.8 Hz, 1H), 8.40 (s, 1H), 11.15 (s, 1H).

MS (ESI⁺) m/z: 480 (MH⁺).

HRMS (ESI⁺) for C₂₅H₂₇FN₅O₄ (MH⁺): calcd, 480.20471. found, 480.20505.

### Example 235

The following compound was prepared consistent with the methods described herein.

### 6-((1-(2-(3-Fluoro-6-(2-(5-hydroxy-4-oxo-1,4-dihydropyridin-2-yl)ethoxy)-1,5-naphthyridin-4-yl)ethyl)-2-oxabicyclo[2.2.2]octan-4-ylamino)methyl)-2H-pyrido[3,2-b][1,4]oxazin-3(4H)-one

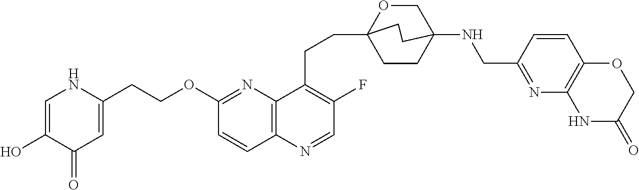 [SMILES: O=C1COc2ccc(CNC34CCC(CCc5c(F)cnc6ccc(OCCc7cc(=O)c(O)c[nH]7)nc56)(CC3)OC4)nc2N1]

¹H NMR (DMSO-d₆) δ 1.55-1.71 (m, 8H), 1.77-1.90 (m, 2H), 3.03 (t, J=6.7 Hz, 2H), 3.13-3.06 (m, 2H), 3.56 (s, 2H), 3.62 (s, 2H), 4.59 (s, 2H), 4.70 (t, J=7.0 Hz, 2H), 6.18 (brs, 1H), 7.01 (d, J=7.9 Hz, 1H), 7.20 (d, J=9.2 Hz, 1H), 7.28 (d, J=7.9 Hz, 1H), 7.30 (brs, 1H), 8.28 (d, J=8.6 Hz, 1H), 8.74 (s, 1H).

MS (ESI⁺) m/z: 617 (MH⁺).

HRMS (ESI⁺) for C₄₆H₄₆FN₆O₆ (MH⁺): calcd, 617.25238. found, 617.25305.

### Example 236

The following compound was prepared consistent with the methods described herein.

### 7-((1-(2-(3-Fluoro-6-methoxy-1,5-naphthyridin-4-yl)ethyl)-2-oxabicyclo[2.2.2]octan-4-ylamino)methyl)-1H-pyrido[2,3-b][1,4]oxazin-2(3H)-one Hydrochloride

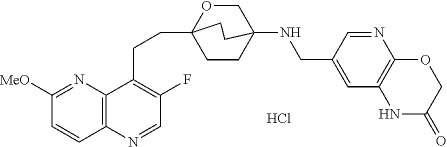 [SMILES: COc1ccc2ncc(F)c(CCC34CCC(NCc5cnc6c(c5)NC(=O)CO6)(CC3)CO4)c2n1 ; Cl]

¹H NMR (DMSO-d₆) δ 1.65-1.76 (m, 2H), 1.78-1.91 (m, 2H), 1.93-2.12 (m, 6H), 3.09-3.17 (m, 2H), 3.91 (s, 2H), 4.01-4.14 (m, 5H), 4.81 (s, 2H), 7.24 (d, J=9.1 Hz, 1H), 7.39 (s, 1H), 7.94 (s, 1H), 8.27 (d, J=9.1 Hz, 1H), 8.76 (s, 1H), 9.34 (brs, 2H), 11.13 (s, 1H).

MS (ESI⁺) m/z: 494 (MH⁺) (as free base).

HRMS (ESI⁺) for C₂₆H₂₉FN₅O₄ (MH⁺) (as free base): calcd, 494.22036. found, 494.22037.

### Example 237

The following compound was prepared consistent with the methods described herein.

### 6-((1-(2-(6-((3R,4S)-4-Aminotetrahydrofuran-3-yloxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

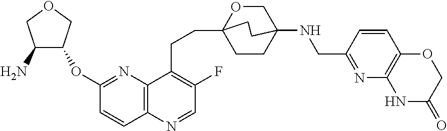 [SMILES: N[C@H]1COC[C@@H]1Oc1ccc2ncc(F)c(CCC34CCC(NCc5ccc6c(n5)NC(=O)CO6)(CC3)CO4)c2n1]

¹H NMR (DMSO-d₆) δ 1.54-1.78 (m, 8H), 1.80-1.95 (m, 2H), 3.01-3.18 (m, 2H), 3.50 (dd, J=8.6, 3.1 Hz, 1H), 3.60 (s, 2H), 3.64 (s, 2H), 3.84 (dd, J=10.4, 1.2 Hz, 1H), 4.00 (dd, J=8.6, 5.5 Hz, 1H), 4.24 (dd, J=10.4, 4.9 Hz, 2H), 4.59 (s, 2H), 5.27 (d, J=4.3 Hz, 1H), 7.23 (d, J=9.2 Hz, 1H), 7.28 (d, J=7.9 Hz, 1H), 8.28 (d, J=9.2 Hz, 1H), 8.76 (s, 1H), 11.14 (s, 1H).

MS (ESI⁺) m/z: 565 (MH⁺).

HRMS (ESI⁺) for C₂₉H₃₄FN₆O₅ (MH⁺): calcd, 565.25747. found, 565.25810.

### Example 238

The following compound was prepared consistent with the methods described herein.

### 8-Chloro-3-((1-(2-(3-fluoro-6-methoxy-1,5-naphthyridin-4-yl)ethyl)-2-oxabicyclo[2.2.2]octan-4-ylamino)methyl)-1-methylquinolin-2(1H)-one

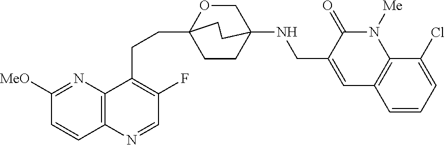 [SMILES: COc1ccc2ncc(F)c(CCC34CCC(NCc5cc6cccc(Cl)c6n(C)c5=O)(CC3)CO4)c2n1]

¹H NMR (DMSO-d₆) δ 1.60-1.80 (m, 8H), 1.84-1.95 (m, 2H), 1.95-2.02 (m, 1H), 3.08-3.17 (m, 2H), 3.56 (brd, J=6.7 Hz, 2H), 3.62 (brs, 2H), 3.85 (s, 3H), 7.22 (d, J=9.2 Hz, 1H), 7.24 (t, J=7.3 Hz, 1H), 7.62 (dd, J=7.9, 1.2 Hz, 1H), 7.69 (dd, J=7.9, 1.8 Hz, 1H), 7.88 (s, 1H), 8.26 (d, J=8.6 Hz, 1H), 8.74 (s, 1H).

MS (ESI⁺) m/z: 537 (MH⁺).

HRMS (ESI⁺) for C₂₉H₃₁ClFN₄O₃ (MH⁺): calcd, 537.20687. found 537.20622.

### Example 239

### (E)-6-Methoxy-4-(2-(4-((3-oxo-3,4-dihydro-2H-pyrido[3,2-b][1,4]oxazin-6-yl)methylamino)-2-oxabicyclo[2.2.2]octan-1-yl)vinyl)pyrido[3,2-c]pyridazine-3-carbonitrile

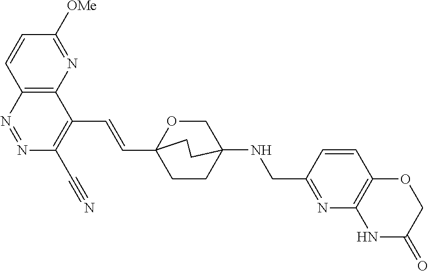 [SMILES: COc1ccc2nnc(C#N)c(/C=C/C34CCC(NCc5ccc6c(n5)NC(=O)CO6)(CC3)CO4)c2n1]

The title compound (12.1 mg) was prepared from (E)-4-(2-(4-amino-2-oxabicyclo[2.2.2]octan-1-yl)vinyl)-6-methoxypyrido[3,2-c]pyridazine-3-carbonitrile (17.1 mg) and 3-oxo-3,4-dihydro-2H-pyrido[3,2-b][1,4]oxazine-6-carbaldehyde (9.9 mg) in the same manner as described for Step 3 of EXAMPLE 1.

¹H NMR (DMSO-d₆) δ 1.66-2.06 (m, 9H), 3.63-3.69 (m, 2H), 3.76 (s, 2H), 4.09 (s, 3H), 4.59 (s, 2H), 7.00-7.05 (m, 2H), 7.29 (d, J=8.0 Hz, 1H), 7.60 (d, J=9.2 Hz, 1H), 7.85 (d, J=15.9 Hz, 1H), 8.76 (d, J=9.2 Hz, 1H), 11.15 (s, 1H).

MS (ESI⁺) m/z: 500 (MH⁺)

HRMS (ESI⁺) for C₂₆H₂₆N₇O₄ (MH⁺): calcd, 500.20463. found, 500.20493.

### Preparation of Intermediates

### Step 1

### Preparation of 3-(3-Fluoro-6-methoxypyridin-2-yl)-3-oxopropanenitrile

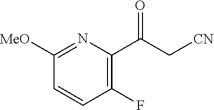 [SMILES: COc1ccc(F)c(C(=O)CC#N)n1]

A solution of 3-fluoro-6-methoxypicolinic acid (2.65 g) in thionyl chloride (11.0 mL) was stirred at 90° C. for 1.5 hours and concentrated in vacuo gave acid chloride. To a solution of cyanoacetic acid (2.78 g) in tetrahydrofuran (50 mL) was added a solution of butyl lithium (23.3 mL, 2.66 M in hexane) at −70° C., the mixture was stirred at the same temperature for 1.5 hours. The resulting solution was added a solution of the above acid chloride as a solution in tetrahydrofuran (32 mL) at −70° C., the mixture was stirred at room temperature for 1 hour. After quenching the reaction by adding hydrochloric acid (62 mL, 1M), the mixture was diluted with ethyl acetate. The mixture was washed with brine, dried over anhydrous sodium sulfate, filtered, and then concentrated in vacuo. Flash chromatography (silica, hexane:ethyl acetate=2:1) of the residue gave the title compound (1.41 g).

¹H NMR (CDCl₃) δ 3.99 (s, 3H), 4.20 (s, 2H), 7.05 (dd, J=9.2, 3.1 Hz, 1H), 7.51 (t, J=9.2 Hz, 1H).

MS (EI⁺) m/z: 194 (M⁺).

HRMS (EI⁺) for C₉H₇FN₂O₂ (M⁺): calcd, 194.0492. found, 194.0500.

### Step 2

### Preparation of 2-Diazo-3-(3-fluoro-6-methoxypyridin-2-yl)-3-oxopropanenitrile

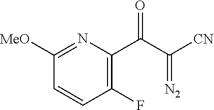

To a solution of 3-(3-fluoro-6-methoxypyridin-2-yl)-3-oxopropanenitrile (400 mg) and pyridine (0.33 mL) in acetonitrile (4.7 mL) was added 1H-imidazole-1-sulfonyl azide (647 mg) under cooling with ice bath, the mixture was stirred at room temperature for 40 minutes, and concentrated in vacuo. Flash chromatography (silica, hexane:ethyl acetate=2:1) of the residue gave the title compound (420 mg).

¹H NMR (CDCl₃) δ 4.01 (s, 3H), 7.02 (dd, J=9.2, 3.1 Hz, 1H), 7.51 (t, J=9.2 Hz, 1H).

MS (EI⁺) m/z: 220 (M⁺).

HRMS (EI⁺) for C₉H₅FN₄O₂ (M⁺): calcd, 220.0397. found, 220.0422.

### Step 3

### Preparation of 2-(3-Fluoro-6-methoxypyridin-2-yl)-2-oxoacetohydrazonoyl cyanide

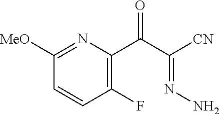 [SMILES: COc1ccc(F)c(C(=O)/C(C#N)=N/N)n1]

To a solution of 2-diazo-3-(3-fluoro-6-methoxypyridin-2-yl)-3-oxopropanenitrile (1.29 g) in tetrahydrofuran (29.5 mL) was added triphenylphosphine (1.70 g), the mixture was stirred at room temperature for 9 hours. Water (3.0 mL) was added to the solution, the mixture was heated under reflux for 7 hours, and then concentrated in vacuo. Flash chromatography (silica, hexane:ethyl acetate=1:1) of the residue gave the title compound (1.10 g).

MS (EI⁺) m/z: 222 (M⁺).

HRMS (EI⁺) for C₉H₇FN₄O₂ (M⁺): calcd, 222.0553. found, 222.0568.

### Step 4

### Preparation of 4-Hydroxy-6-methoxypyrido[3,2-c]pyridazine-3-carbonitrile

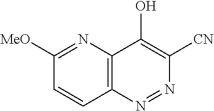 [SMILES: COc1ccc2nnc(C#N)c(O)c2n1]

A solution of 2-(3-fluoro-6-methoxypyridin-2-yl)-2-oxoacetohydrazonoyl cyanide (301 mg) in diglyme (13.5 mL) was stirred at 140° C. for 7 hours. After cooling the mixture with ice bath, the resulting precitipates were collected by filtration to give the title compound (80.0 mg).

¹H NMR (DMSO-d₆) δ 3.99 (s, 3H), 7.41 (d, J=9.1 Hz, 1H), 8.09 (d, J=9.1 Hz, 1H), 14.66 (brs, 1H).

MS (EI⁺) m/z: 202 (M⁺).

HRMS (EI⁺) for C₉H₆N₄O₂ (M⁺): calcd, 202.0491. found, 202.0461.

### Step 5

### Preparation of 4-Bromo-6-methoxypyrido[3,2-c]pyridazine-3-carbonitrile

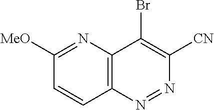 [SMILES: COc1ccc2nnc(C#N)c(Br)c2n1]

The title compound (100 mg) was prepared from 4-hydroxy-6-methoxypyrido[3,2-c]pyridazine-3-carbonitrile (85.0 mg) in the same manner as described for Intermediate J.

¹H NMR (CDCl₃) δ 4.17 (s, 3H), 7.67 (d, J=9.2 Hz, 1H), 8.82 (d, J=9.2 Hz, 1H).

MS (EI⁺) m/z: 264 (M⁺).

HRMS (EI⁺) for C₉H₅BrN₄O (M⁺): calcd, 263.9647. found, 263.9662.

### Step 6

### Preparation of (E)-tert-Butyl 1-(2-(3-Cyano-6-methoxypyrido[3,2-c]pyridazin-4-yl)vinyl)-2-oxabicyclo[2.2.2]octan-4-ylcarbamate

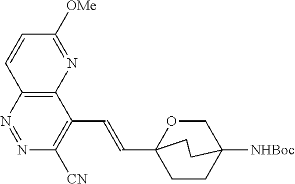 [SMILES: COc1ccc2nnc(C#N)c(/C=C/C34CCC(NC(=O)OC(C)(C)C)(CC3)CO4)c2n1]

The title compound (35.4 mg) was prepared from 4-bromo-6-methoxypyrido[3,2-c]pyridazine-3-carbonitrile (59.0 mg) and tert-butyl 1-vinyl-2-oxabicyclo[2.2.2]octan-4-ylcarbamate (57.3 mg) in the same manner as described for Step 1 of EXAMPLE 18.

¹H NMR (DMSO-d₆) δ 1.37 (s, 9H), 1.82-2.10 (m, 8H), 3.95 (s, 2H), 4.09 (s, 3H), 6.70 (brs, 1H), 7.02 (d, J=15.9 Hz, 1H), 7.60 (d, J=9.2 Hz, 1H), 7.81 (d, J=15.9 Hz, 1H), 8.76 (d, J=9.2 Hz, 1H).

MS (ESI⁺) m/z: 438 (MH⁺).

HRMS (ESI⁺) for C₂₃H₂₈N₅O₄ (MH⁺): calcd, 438.21413. found, 438.21431.

### Step 7

### Preparation of (E)-4-(2-(4-Amino-2-oxabicyclo[2.2.2]octan-1-yl)vinyl)-6-methoxypyrido[3,2-c]pyridazine-3-carbonitrile

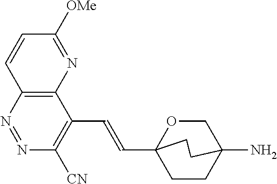 [SMILES: COc1ccc2nnc(C#N)c(/C=C/C34CCC(N)(CC3)CO4)c2n1]

The title compound (19.1 mg) was prepared from (E)-tert-butyl 1-(2-(3-cyano-6-methoxypyrido[3,2-c]pyridazin-4-yl)vinyl)-2-oxabicyclo[2.2.2]octan-4-ylcarbamate (30.2 mg) in the same manner as described for Step 2 of EXAMPLE 1.

¹H NMR (DMSO-d₆) δ 1.43 (brs, 2H), 1.48-1.71 (m, 4H), 1.79-1.89 (m, 2H) 1.92-2.02 (m, 2H), 3.62 (s, 2H), 4.09 (s, 3H), 7.01 (d, J=15.9 Hz, 1H), 7.60 (d, J=9.2 Hz, 1H), 7.84 (d, J=15.9 Hz, 1H), 8.76 (d, J=9.2 Hz, 1H).

MS (ESI⁺) m/z: 338 (MH⁺).

HRMS (ESI⁺) for C₁₈H₂₀N₅O₂ (MH⁺): calcd, 338.16170. found, 338.16186.

### Example 240

The following compound was prepared consistent with the methods described herein.

### 6-((1-(1-Hydroxy-2-(7-(2-hydroxyethoxy)-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one (Enantiomer A)

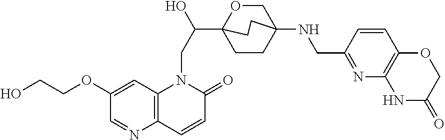 [SMILES: O=C1COc2ccc(CNC34CCC(C(O)Cn5c(=O)ccc6ncc(OCCO)cc65)(CC3)OC4)nc2N1]

¹H NMR (DMSO-d₆) δ 1.59-2.05 (m, 8H), 3.52-3.60 (m, 1H), 3.64 (s, 4H), 3.77 (dd, J=10.4, 4.9 Hz, 1H), 4.10-4.18 (m, 2H), 4.24-4.34 (m, 2H), 4.59 (s, 2H), 4.91 (d, J=6.1 Hz, 1H), 4.98 (t, J=5.5 Hz, 1H), 6.64 (d, J=9.8 Hz, 1H), 7.02 (d, J=8.0 Hz, 1H), 7.28 (d, J=8.6 Hz, 1H), 7.52 (d, J=2.4 Hz, 1H), 7.84 (d, J=9.8 Hz, 1H), 8.24 (d, J=2.4 Hz, 1H), 11.15 (s, 1H).

MS (ESI⁺) m/z: 538 (MH⁺).

HRMS (ESI⁺) for C₂₇H₃₂N₅O₇ (MH⁺): calcd, 538.23017. found, 538.22999.

### Example 241

The following compound was prepared consistent with the methods described herein.

### 6-((1-(1-Hydroxy-2-(7-(2-hydroxyethoxy)-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one (Enantiomer B)

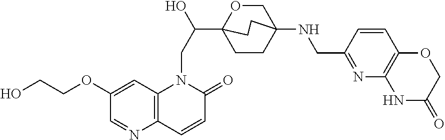 [SMILES: O=C1COc2ccc(CNC34CCC(C(O)Cn5c(=O)ccc6ncc(OCCO)cc65)(CC3)OC4)nc2N1]

¹H NMR (DMSO-d₆) δ 1.55-2.05 (m, 8H), 3.52-3.60 (m, 1H), 3.64 (s, 4H), 3.77 (dd, J=9.8, 4.9 Hz, 1H), 4.10-4.20 (m, 2H), 4.24-4.34 (m, 2H), 4.59 (s, 2H), 4.90 (d, J=6.1 Hz, 1H), 4.98 (t, J=5.5 Hz, 1H), 6.64 (d, J=9.8 Hz, 1H), 7.02 (d, J=8.0 Hz, 1H), 7.28 (d, J=8.0 Hz, 1H), 7.52 (d, J=1.8 Hz, 1H), 7.84 (d, J=9.8 Hz, 1H), 8.24 (d, J=2.4 Hz, 1H), 11.15 (s, 1H).

MS (ESI⁺) m/z: 538 (MH⁺).

HRMS (ESI⁺) for C₂₇H₃₂N₅O₇ (MH⁺): calcd, 538.23017. found, 538.23038.

### Example 242

### 6-Methoxy-4-(2-(4-((3-oxo-3,4-dihydro-2H-pyrido[3,2-b][1,4]oxazin-6-yl)methylamino)-2-oxabicyclo[2.2.2]octan-1-yl)ethyl)pyrido[3,2-c]pyridazine-3-carbonitrile

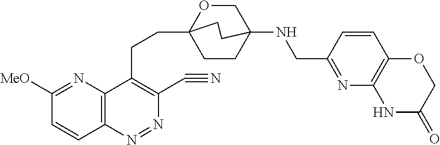 [SMILES: COc1ccc2nnc(C#N)c(CCC34CCC(NCc5ccc6c(n5)NC(=O)CO6)(CC3)CO4)c2n1]

The title compound (25.6 mg) was prepared from 4-(2-(4-amino-2-oxabicyclo[2.2.2]octan-1-yl)ethyl)-6-methoxypyrido[3,2-c]pyridazine-3-carbonitrile (20.1 mg) and 3-oxo-3,4-dihydro-2H-pyrido[3,2-b][1,4]oxazine-6-carbaldehyde (10.5 mg) in the same manner as described for Step 3 of EXAMPLE 1.

¹H NMR (DMSO-d₆) δ 1.58-1.79 (m, 8H), 1.83-1.96 (m, 3H), 3.22-3.30 (m, 2H), 3.54 (s, 2H), 3.61 (s, 2H), 4.11 (s, 3H), 4.58 (s, 2H), 7.00 (d, J=8.6 Hz, 1H), 7.27 (d, J=8.0 Hz, 1H), 7.58 (d, J=9.2 Hz, 1H), 8.76 (d, J=9.2 Hz, 1H), 11.14 (s, 1H).

MS (ESI⁺) m/z: 502 (MH⁺).

HRMS (ESI⁺) for C₂₆H₂₈N₇O₄ (MH⁺): calcd, 502.22028. found, 502.22039.

### Preparation of Intermediates

### Step 1

### Preparation of tert-Butyl 1-(2-(3-Cyano-6-methoxypyrido[3,2-c]pyridazin-4-yl)ethyl)-2-oxabicyclo[2.2.2]octan-4-ylcarbamate

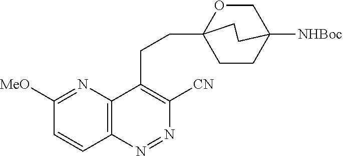 [SMILES: COc1ccc2nnc(C#N)c(CCC34CCC(NC(=O)OC(C)(C)C)(CC3)CO4)c2n1]

The title compound (40.5 mg) was prepared from (E)-tert-butyl 1-(2-(3-cyano-6-methoxypyrido[3,2-c]pyridazin-4-yl)vinyl)-2-oxabicyclo[2.2.2]octan-4-ylcarbamate (50.1 mg) in the same manner as described for Step 2 of EXAMPLE 18.

¹H NMR (DMSO-d₆) δ 1.35 (s, 9H), 1.66-1.99 (m, 10H), 3.21-3.28 (m, 2H), 3.73 (s, 2H), 4.11 (s, 3H), 6.57 (brs, 1H), 7.58 (d, J=9.2 Hz, 1H), 8.75 (d, J=9.2 Hz, 1H).

MS (ESI⁺) m/z: 440 (MH⁺).

HRMS (ESI⁺) for C₂₃H₃₀N₅O₄ (MH⁺): calcd, 440.22978. found, 440.22950.

### Step 2

### Preparation of 4-(2-(4-Amino-2-oxabicyclo[2.2.2]octan-1-yl)ethyl)-6-methoxypyrido[3,2-c]pyridazine-3-carbonitrile

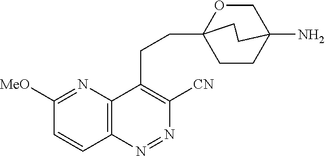 [SMILES: COc1ccc2nnc(C#N)c(CCC34CCC(N)(CC3)CO4)c2n1]

The title compound (26.1 mg) was prepared from tert-butyl 1-(2-(3-cyano-6-methoxypyrido[3,2-c]pyridazin-4-yl)ethyl)-2-oxabicyclo[2.2.2]octan-4-ylcarbamate (34.0 mg) in the same manner as described for Step 2 of EXAMPLE 1.

¹H NMR (DMSO-d₆) δ 1.36 (brs, 2H), 1.47-1.78 (m, 8H), 1.82-1.92 (m, 2H) 3.21-3.30 (m, 2H), 3.40 (s, 2H), 4.11 (s, 3H), 7.58 (d, J=9.2 Hz, 1H), 8.75 (d, J=9.2 Hz, 1H).

MS (ESI⁺) m/z: 340 (MH⁺).

HRMS (ESI⁺) for C₁₈H₂₂N₅O₂ (MH⁺): calcd, 340.17735. found, 340.17765.

### Example 243

### 6-((1-(1-Hydroxy-2-(7-(3-hydroxypropoxy)-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one (Enantiomer A)

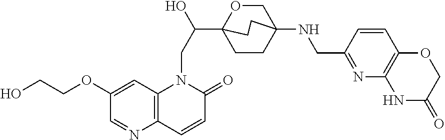 [SMILES: O=C1COc2ccc(CNC34CCC(C(O)Cn5c(=O)ccc6ncc(OCCO)cc65)(CC3)OC4)nc2N1]

¹H NMR (DMSO-d₆) δ 1.59-2.00 (m, 10H), 3.53-3.68 (m, 7H), 4.15-4.24 (m, 2H), 4.26-4.33 (m, 2H), 4.59-4.62 (m, 3H), 4.91 (d, J=6.1 Hz, 1H), 6.63 (d, J=9.8 Hz, 1H), 7.02 (d, J=7.9 Hz, 1H), 7.28 (d, J=7.9 Hz, 1H), 7.53 (d, J=1.8 Hz, 1H), 7.83 (d, J=9.8 Hz, 1H), 8.22 (d, J=2.4 Hz, 1H), 11.15 (s, 1H).

MS (ESI⁺) m/z: 552 (MH⁺).

HRMS (ESI⁺) for C₂₈H₃₄N₅O₇ (MH⁺): calcd, 552.24582. found: 552.24496.

### Preparation of Intermediates

### Step 1

### Preparation of tert-Butyl 1-(1-Hydroxy-2-(2-oxo-7-(3-(tetrahydro-2H-pyran-2-yloxy)propoxy)-1,5-naphthyridin-1(2H)-yl)ethyl)-2-oxabicyclo[2.2.2]octan-4-ylcarbamate (Enantiomer A)

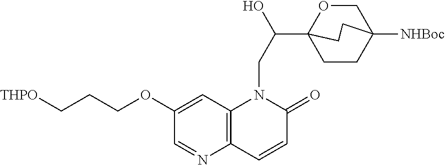

The title compound (117 mg) was prepared from tert-butyl 1-(1-hydroxy-2-(7-hydroxy-2-oxo-1,5-naphthyridin-1(2H)-yl)ethyl)-2-oxabicyclo[2.2.2]octan-4-ylcarbamate (100 mg) and 2-(3-bromopropoxy)tetrahydro-2H-pyran in the same manner as described for Step 1 of EXAMPLE 32.

¹H NMR (CDCl₃): δ 1.44 (s, 9H), 1.50-2.24 (m, 16H), 3.46-3.54 (m, 1H), 3.58-3.64 (m, 1H), 3.65-3.70 (m, 1H), 3.82-3.90 (m, 1H), 3.96-4.03 (m, 2H), 4.08 (brs, 1H), 4.10-4.16 (m, 1H), 4.10-4.16 (m, 1H), 4.22 (t, J=6.1 Hz, 2H) 4.33-4.48 (m, 3H), 4.59-4.62 (m, 1H), 6.76 (d, J=9.8 Hz, 1H), 7.52 (d, J=2.4 Hz, 1H), 7.90 (d, J=9.8 Hz, 1H), 8.29 (d, J=2.4 Hz, 1H)

MS (ESI⁺) m/z: 574 (MH⁺).

HRMS (ESI⁺) for C₃₀H₄₄N₃O₈ (MH⁺): calcd, 574.31284. found: 574.31212.

### Step 2

### Preparation of 1-(2-(4-Amino-2-oxabicyclo[2.2.2]octan-1-yl)-2-hydroxyethyl)-7-(3-hydroxypropoxy)-1,5-naphthyridin-2(1H)-one

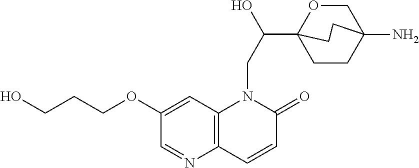 [SMILES: NC12CCC(C(O)Cn3c(=O)ccc4ncc(OCCCO)cc43)(CC1)OC2]

The title compound (74.0 mg) was prepared from tert-butyl 1-(1-hydroxy-2-(2-oxo-7-(3-(tetrahydro-2H-pyran-2-yloxy)propoxy)-1,5-naphthyridin-1(2H)-yl)ethyl)-2-oxabicyclo[2.2.2]octan-4-ylcarbamate (100 mg) in the same manner as described for Step 2 of EXAMPLE 32.

¹H NMR (DMSO-d₆): δ 1.51-1.99 (m, 10H), 3.51-3.61 (m, 5H), 4.16-4.32 (m, 4H), 4.60 (t, J=4.9 Hz, 1H), 4.90 (d, J=6.1 Hz, 1H), 6.63 (d, J=9.8 Hz, 1H), 7.52 (d, J=2.4 Hz, 1H), 7.83 (d, J=9.8 Hz, 1H), 8.22 (d, J=2.4 Hz, 1H).

MS (ESI⁺) m/z: 390 (MH⁺).

HRMS (ESI⁺) for C₂₀H₂₈N₃O₅ (MH⁺): calcd, 390.20290. found: 390.20270.

### Example 244

### 6-((1-(1-Hydroxy-2-(7-(3-hydroxypropoxy)-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one (Enantiomer B)

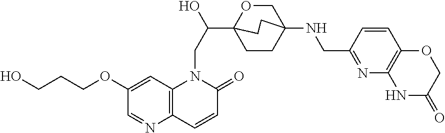 [SMILES: O=C1COc2ccc(CNC34CCC(C(O)Cn5c(=O)ccc6ncc(OCCCO)cc65)(CC3)OC4)nc2N1]

The title compound (68.3 mg) was prepared from 1-(2-(4-amino-2-oxabicyclo[2.2.2]octan-1-yl)-2-hydroxyethyl)-7-(3-hydroxypropoxy)-1,5-naphthyridin-2(1H)-one (79.0 mg) and 3-oxo-3,4-dihydro-2H-pyrido[3,2-b][1,4]oxazine-6-carbaldehyde (36.0 mg) in the same manner as described for Step 3 of EXAMPLE 1.

¹H NMR (DMSO-d₆) δ 1.59-2.00 (m, 10H), 3.53-3.68 (m, 7H), 4.15-4.24 (m, 2H), 4.26-4.33 (m, 2H), 4.59-4.62 (m, 3H), 4.91 (d, J=6.1 Hz, 1H), 6.63 (d, J=9.8 Hz, 1H), 7.02 (d, J=7.9 Hz, 1H), 7.28 (d, J=7.9 Hz, 1H), 7.53 (d, J=1.8 Hz, 1H), 7.83 (d, J=9.8 Hz, 1H), 8.22 (d, J=2.4 Hz, 1H), 11.15 (s, 1H).

MS (ESI⁺) m/z: 552 (MH⁺).

HRMS (ESI⁺) for C₂₈H₃₄N₅O₇ (MH⁺): calcd, 552.24582. found: 552.24486.

### Preparation of Intermediates

### Step 1

### Preparation of tert-Butyl 1-(1-Hydroxy-2-(2-oxo-7-(3-(tetrahydro-2H-pyran-2-yloxy)propoxy)-1,5-naphthyridin-1(2H)-yl)ethyl)-2-oxabicyclo[2.2.2]octan-4-ylcarbamate

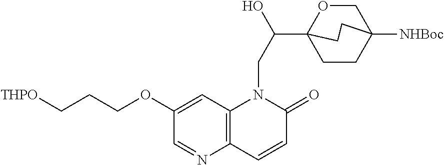

The title compound (139 mg) was prepared from tert-butyl 1-(1-hydroxy-2-(7-hydroxy-2-oxo-1,5-naphthyridin-1(2H)-yl)ethyl)-2-oxabicyclo[2.2.2]octan-4-ylcarbamate (115 mg) and 2-(3-bromopropoxy)tetrahydro-2H-pyran in the same manner as described for Step 1 of EXAMPLE 32.

¹H NMR (CDCl₃): δ 1.44 (s, 9H), 1.50-2.24 (m, 16H), 3.46-3.54 (m, 1H), 3.58-3.64 (m, 1H), 3.65-3.70 (m, 1H), 3.82-3.90 (m, 1H), 3.96-4.03 (m, 2H), 4.08 (brs, 1H), 4.10-4.16 (m, 1H), 4.10-4.16 (m, 1H), 4.22 (t, J=6.1 Hz, 2H) 4.33-4.48 (m, 3H), 4.59-4.62 (m, 1H), 6.76 (d, J=9.8 Hz, 1H), 7.52 (d, J=2.4 Hz, 1H), 7.90 (d, J=9.8 Hz, 1H), 8.29 (d, J=2.4 Hz, 1H)

MS (ESI⁺) m/z: 574 (MH⁺).

HRMS (ESI⁺) for C₃₀H₄₄N₃O₈ (MH⁺): calcd, 574.31284. found: 574.31213.

### Step 2

### Preparation of 1-(2-(4-Amino-2-oxabicyclo[2.2.2]octan-1-yl)-2-hydroxyethyl)-7-(3-hydroxypropoxy)-1,5-naphthyridin-2(1H)-one

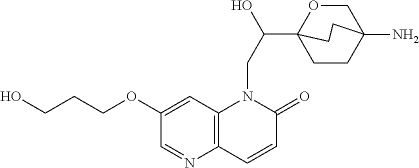 [SMILES: NC12CCC(C(O)Cn3c(=O)ccc4ncc(OCCCO)cc43)(CC1)OC2]

The title compound (89.5 mg) was prepared from tert-butyl 1-(1-hydroxy-2-(2-oxo-7-(3-(tetrahydro-2H-pyran-2-yloxy)propoxy)-1,5-naphthyridin-1(2H)-yl)ethyl)-2-oxabicyclo[2.2.2]octan-4-ylcarbamate (120 mg) in the same manner as described for Step 2 of EXAMPLE 32.

¹H NMR (DMSO-d₆): δ 1.51-1.99 (m, 10H), 3.51-3.61 (m, 5H), 4.16-4.32 (m, 4H), 4.60 (t, J=4.9 Hz, 1H), 4.90 (d, J=6.1 Hz, 1H), 6.63 (d, J=9.8 Hz, 1H), 7.52 (d, J=2.4 Hz, 1H), 7.83 (d, J=9.8 Hz, 1H), 8.22 (d, J=2.4 Hz, 1H).

MS (ESI⁺) m/z: 390 (MH⁺).

HRMS (ESI⁺) for C₂₀H₂₈N₃O₅ (MH⁺): calcd, 390.20290. found: 390.20257.

### Example 245

The following compound was prepared consistent with the methods described herein.

### 6-((1-(2-(6-((3S,4S)-4-Amino-5-oxopyrrolidin-3-yloxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

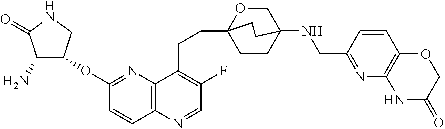 [SMILES: N[C@@H]1C(=O)NC[C@@H]1Oc1ccc2ncc(F)c(CCC34CCC(NCc5ccc6c(n5)NC(=O)CO6)(CC3)CO4)c2n1]

¹H NMR (DMSO-d₆) δ 1.61-2.15 (m, 10H), 2.97-3.14 (m, 2H), 3.24 (dd, J=11.0, 2.4 Hz, 1H), 3.51 (dd, J=11.0, 3.7 Hz, 1H), 3.60-3.84 (m, 3H), 4.61-4.75 (m, 3H), 4.89 (dd, J=6.7, 4.9 Hz, 1H), 5.31 (d, J=4.3 Hz, 1H), 7.03-7.21 (m, 1H), 7.33 (d, J=9.2 Hz, 1H), 7.36-7.46 (m, 1H), 7.47-7.61 (m, 1H), 7.93 (s, 1H), 7.97 (d, J=9.2 Hz, 1H), 8.50 (s, 1H), 11.26 (s, 1H).

MS (ESI⁺) m/z: 578 (MH⁺).

HRMS (ESI⁺) for C₂₉H₃₃FN₇O₅ (MH⁺): calcd, 578.25272. found, 578.25209.

### Example 246

### Methyl 2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylate (Enantiomer A)

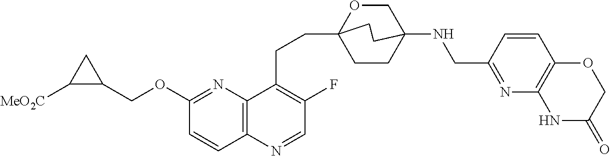 [SMILES: COC(=O)C1CC1COc1ccc2ncc(F)c(CCC34CCC(NCc5ccc6c(n5)NC(=O)CO6)(CC3)CO4)c2n1]

The title compound (814 mg) was prepared from (1SR,2SR)-methyl 2-((8-(2-(4-amino-2-oxabicyclo[2.2.2]octan-1-yl)ethyl)-7-fluoro-1,5-naphthyridin-2-yloxy)methyl)cyclopropanecarboxylate (800 mg) and 3-oxo-3,4-dihydro-2H-pyrido[3,2-b][1,4]oxazine-6-carbaldehyde (365 mg) in the same manner as described for Step 3 of EXAMPLE 1.

Optical resolution (CHIRALPAK IA, ethyl acetate:heptane:diethylamine=9:1:0.3) of the racemate (800 mg) gave Enantiomer A (378 mg).

¹H NMR (CDCl₃) δ 1.01-1.07 (m, 1H), 1.29-1.35 (m, 1H), 1.70-1.88 (m, 9H), 1.97-2.10 (m, 3H), 3.13-3.22 (m, 2H), 3.70 (s, 3H), 3.76 (s, 2H), 3.78 (s, 2H), 4.33 (dd, J=11.6, 7.3 Hz, 1H), 4.52 (dd, J=11.6, 6.1 Hz, 1H), 4.63 (s, 2H), 6.95 (d, J=7.9 Hz, 1H), 7.06 (d, J=9.2 Hz, 1H), 7.20 (d, J=7.9 Hz, 1H), 8.09 (br, 1H), 8.18 (d, J=9.2 Hz, 1H), 8.60 (s, 1H).

MS (ESI⁺) m/z: 592 (MH⁺).

HRMS (ESI⁺) for C₃₁H₃₅FN₅O₆ (MH⁺): calcd, 592.25714. found, 592.25704.

### Preparation of Intermediates

### Step 1

### Preparation of (1SR,2SR)-Methyl 2-((8-(2-(4-(tert-Butoxycarbonylamino)-2-oxabicyclo[2.2.2]octan-1-yl)ethyl)-7-fluoro-1,5-naphthyridin-2-yloxy)methyl)cyclopropanecarboxylate

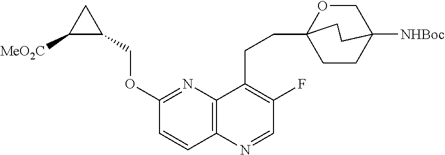 [SMILES: COC(=O)[C@H]1C[C@@H]1COc1ccc2ncc(F)c(CCC34CCC(NC(=O)OC(C)(C)C)(CC3)CO4)c2n1]

The title compound (1.09 g) was prepared from tert-butyl 1-(2-(3-fluoro-6-hydroxy-1,5-naphthyridin-4-yl)ethyl)-2-oxabicyclo[2.2.2]octan-4-ylcarbamate (880 mg) and (1SR,2SR)-methyl 2-(bromomethyl)cyclopropanecarboxylate (448 mg) in the same manner as described for Step 1 of EXAMPLE 32.

¹H NMR (CDCl₃) δ 0.99-1.07 (m, 1H), 1.28-1.35 (m, 1H), 1.44 (s, 9H), 1.68-1.81 (m, 5H), 1.83-1.93 (m, 2H), 1.97-2.16 (m, 5H), 3.10-3.20 (m, 2H), 3.69 (s, 3H), 3.96 (s, 2H), 4.31 (d, J=11.6, 7.3 Hz, 1H), 4.26-4.36 (m, 1H), 4.47 (dd, J=11.6, 6.1 Hz, 1H), 7.05 (d, J=9.2 Hz, 1H), 8.16 (d, J=9.2 Hz, 1H), 8.59 (s, 1H).

MS (ESI⁺) m/z: 530 (MH⁺).

HRMS (ESI⁺) for C₂₈H₃₇FN₃O₆ (MH⁺): calcd, 530.26664. found, 530.26634.

### Step 2

### Preparation of (1SR,2SR)-Methyl 2-((8-(2-(4-Amino-2-oxabicyclo[2.2.2]octan-1-yl)ethyl)-7-fluoro-1,5-naphthyridin-2-yloxy)methyl)cyclopropanecarboxylate

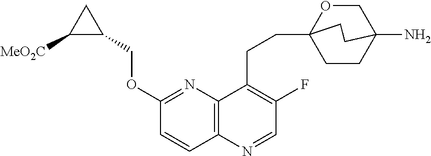 [SMILES: COC(=O)[C@H]1C[C@@H]1COc1ccc2ncc(F)c(CCC34CCC(N)(CC3)CO4)c2n1]

The title compound (820 mg) was prepared from (1SR,2SR)-methyl 2-((8-(2-(4-(tert-butoxycarbonylamino)-2-oxabicyclo[2.2.2]octan-1-yl)ethyl)-7-fluoro-1,5-naphthyridin-2-yloxy)methyl)cyclopropanecarboxylate (1.00 g) in the same manner as described for Step 2 of EXAMPLE 1.

¹H NMR (CDCl₃) δ 1.00-1.07 (m, 1H), 1.28-1.35 (m, 1H), 1.63-1.80 (m, 9H), 1.93-2.08 (m, 3H), 3.12-3.20 (m, 2H), 3.65 (s, 2H), 3.70 (s, 3H), 4.33 (d, J=11.6, 7.3 Hz, 1H), 4.51 (dd, J=11.6, 6.1 Hz, 1H), 7.06 (d, J=9.2 Hz, 1H), 8.17 (d, J=9.2 Hz, 1H), 8.59 (s, 1H).

MS (ESI⁺) m/z: 430 (MH⁺).

HRMS (ESI⁺) for C₂₃H₂₉FN₃O₄ (MH⁺): calcd, 430.21421. found, 430.21492.

### Example 247

The following compound was prepared consistent with the methods described herein.

### Methyl 2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylate (Enantiomer B)

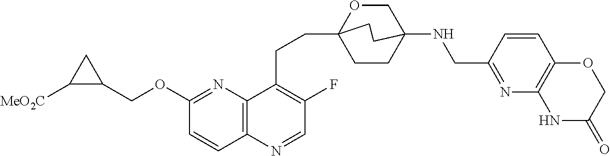 [SMILES: COC(=O)C1CC1COc1ccc2ncc(F)c(CCC34CCC(NCc5ccc6c(n5)NC(=O)CO6)(CC3)CO4)c2n1]

Optical resolution (CHIRALPAK IA, ethyl acetate:heptane:diethylamine=9:1:0.3) of the racemate (800 mg) of EXAMPLE 256 gave Enantiomer B (382 mg).

¹H NMR (CDCl₃) δ 1.01-1.07 (m, 1H), 1.29-1.35 (m, 1H), 1.70-1.88 (m, 9H), 1.97-2.10 (m, 3H), 3.12-3.22 (m, 2H), 3.70 (s, 3H), 3.76 (s, 2H), 3.78 (s, 2H), 4.33 (dd, J=11.6, 7.3 Hz, 1H), 4.52 (dd, J=11.6, 6.1 Hz, 1H), 4.63 (s, 1H), 6.95 (d, J=7.9 Hz, 1H), 7.06 (d, J=9.2 Hz, 1H), 7.20 (d, J=7.9 Hz, 1H), 8.09 (br, 1H), 8.18 (d, J=9.2 Hz, 1H), 8.60 (s, 1H).

MS (ESI⁺) m/z: 592 (MH⁺).

HRMS (ESI⁺) for C₃₁H₃₅FN₅O₆ (MH⁺): calcd, 592.25714. found, 592.25731.

### Example 248

The following compound was prepared consistent with the methods described herein.

### 2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylic Acid (Enantiomer A)

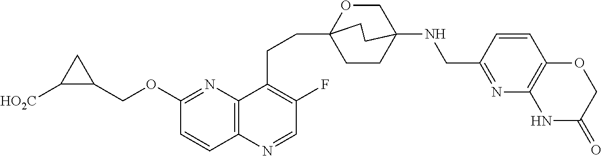 [SMILES: O=C1COc2ccc(CNC34CCC(CCc5c(F)cnc6ccc(OCC7CC7C(=O)O)nc56)(CC3)OC4)nc2N1]

¹H NMR (DMSO-d₆) δ 0.94-1.03 (m, 1H), 1.06-1.13 (m, 1H), 1.55-1.76 (m, 9H), 1.80-1.93 (m, 3H), 3.04-3.13 (m, 2H), 3.59 (s, 2H), 3.63 (s, 2H), 4.35 (dd, J=11.6, 7.4 Hz, 1H), 4.44 (dd, J=11.6, 6.7 Hz, 1H), 4.59 (s, 2H), 7.01 (d, J=7.9 Hz, 1H), 7.24 (d, J=9.2 Hz, 1H), 7.28 (d, J=7.9 Hz, 1H), 8.26 (d, J=9.2 Hz, 1H), 8.74 (s, 1H), 11.15 (s, 1H).

MS (ESI⁺) m/z: 578 (MH⁺).

HRMS (ESI⁺) for C₃₀H₃₃FN₅O₆ (MH⁺): calcd, 578.24149. found, 578.24136.

### Example 249

The following compound was prepared consistent with the methods described herein.

### 2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylic Acid (Enantiomer B)

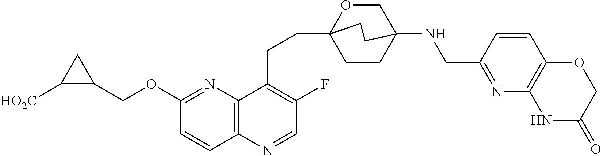 [SMILES: O=C1COc2ccc(CNC34CCC(CCc5c(F)cnc6ccc(OCC7CC7C(=O)O)nc56)(CC3)OC4)nc2N1]

¹H NMR (DMSO-d₆) δ 0.93-1.04 (m, 1H), 1.05-1.11 (m, 1H), 1.55-1.76 (m, 9H), 1.80-1.95 (m, 3H), 3.01-3.14 (m, 2H), 3.59 (s, 2H), 3.63 (s, 2H), 4.35 (dd, J=11.6, 7.4 Hz, 1H), 4.44 (dd, J=11.6, 6.7 Hz, 1H), 4.59 (s, 2H), 7.01 (d, J=7.9 Hz, 1H), 7.24 (d, J=9.2 Hz, 1H), 7.28 (d, J=7.9 Hz, 1H), 8.26 (d, J=9.2 Hz, 1H), 8.74 (s, 1H), 11.15 (s, 1H).

MS (ESI⁺) m/z: 578 (MH⁺).

HRMS (ESI⁺) for C₃₀H₃₃FN₅O₆ (MH⁺): calcd, 578.24149. found, 578.24163.

### Example 250

### Ethyl 4-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanoate

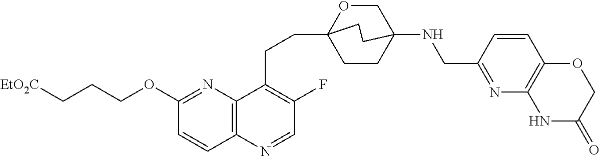 [SMILES: CCOC(=O)CCCOc1ccc2ncc(F)c(CCC34CCC(NCc5ccc6c(n5)NC(=O)CO6)(CC3)CO4)c2n1]

The title compound (124 mg) was prepared from ethyl 4-(8-(2-(4-amino-2-oxabicyclo[2.2.2]octan-1-yl)ethyl)-7-fluoro-1,5-naphthyridin-2-yloxy)butanoate (100 mg) and 3-oxo-3,4-dihydro-2H-pyrido[3,2-b][1,4]oxazine-6-carbaldehyde (43.4 mg) in the same manner as described for Step 3 of EXAMPLE 1.

¹H NMR (CDCl₃) δ 1.26 (t, J=7.3 Hz, 3H), 1.71-1.86 (m, 8H), 1.97-2.07 (m, 2H), 2.19 (quintet, J=6.7 Hz, 2H), 2.54 (t, J=7.3 Hz, 2H), 3.14-3.22 (m, 2H), 3.76 (s, 2H), 3.77 (s, 2H), 4.16 (q, J=7.3 Hz, 2H), 4.54 (t, J=6.1 Hz, 2H), 4.63 (s, 2H), 6.94 (d, J=7.9 Hz, 1H), 7.03 (d, J=9.2 Hz, 1H), 7.20 (d, J=7.9 Hz, 1H), 8.06 (br, 1H), 8.16 (d, J=9.2 Hz, 1H), 8.59 (s, 1H).

MS (ESI⁺) m/z: 594 (MH⁺).

HRMS (ESI⁺) for C₃₁H₃₇FN₅O₆ (MH⁺): calcd, 594.27279. found, 594.27195.

### Preparation of Intermediates

### Step 1

### Preparation of Ethyl 4-(8-(2-(4-(tert-Butoxycarbonylamino)-2-oxabicyclo[2.2.2]octan-1-yl)ethyl)-7-fluoro-1,5-naphthyridin-2-yloxy)butanoate

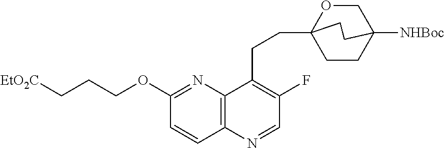 [SMILES: CCOC(=O)CCCOc1ccc2ncc(F)c(CCC34CCC(NC(=O)OC(C)(C)C)(CC3)CO4)c2n1]

The title compound (140 mg) was prepared from tert-butyl 1-(2-(3-fluoro-6-hydroxy-1,5-naphthyridin-4-yl)ethyl)-2-oxabicyclo[2.2.2]octan-4-ylcarbamate (120 mg) and ethyl 4-bromobutanoate (61.7 mg) in the same manner as described for Step 1 of EXAMPLE 32.

¹H NMR (CDCl₃) δ 1.26 (t, J=7.3 Hz, 3H), 1.43 (s, 9H), 1.67-1.81 (m, 4H), 1.82-1.95 (m, 2H), 1.97-2.16 (m, 4H), 2.16-2.24 (m, 2H), 2.53 (t, J=7.3 Hz, 2H), 3.12-3.21 (m, 2H), 3.96 (s, 2H), 4.16 (q, J=7.3 Hz, 2H), 4.28 (br, s, 1H), 4.53 (t, J=6.1 Hz, 2H), 7.03 (d, J=8.6 Hz, 1H), 8.15 (d, J=9.2 Hz, 1H), 8.58 (s, 1H).

MS (ESI⁺) m/z: 532 (MH⁺).

HRMS (ESI⁺) for C₂₈H₃₉FN₃O₆ (MH⁺): calcd, 532.28229. found, 532.28140.

### Step 2

### Preparation of Ethyl 4-(8-(2-(4-Amino-2-oxabicyclo[2.2.2]octan-1-yl)ethyl)-7-fluoro-1,5-naphthyridin-2-yloxy)butanoate

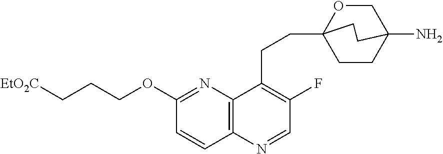 [SMILES: CCOC(=O)CCCOc1ccc2ncc(F)c(CCC34CCC(N)(CC3)CO4)c2n1]

The title compound (104 mg) was prepared from ethyl 4-(8-(2-(4-(tert-butoxycarbonylamino)-2-oxabicyclo[2.2.2]octan-1-yl)ethyl)-7-fluoro-1,5-naphthyridin-2-yloxy)butanoate (135 mg) in the same manner as described for Step 2 of EXAMPLE 32.

¹H NMR (CDCl₃) δ: 1.26 (t, J=7.3 Hz, 3H), 1.62-1.83 (m, 8H), 1.95-2.06 (m, 2H), 2.19 (quintet, J=7.3 Hz, 2H), 2.54 (t, J=7.3 Hz, 2H), 3.13-3.21 (m, 2H), 3.66 (s, 2H), 4.16 (q, J=7.3 Hz, 2H), 4.53 (t, J=6.7 Hz, 2H), 7.03 (d, J=9.2 Hz, 1H), 8.16 (d, J=9.2 Hz, 1H), 8.59 (s, 1H).

MS (ESI⁺) m/z: 432 (MH⁺).

HRMS (ESI⁺) for C₂₃H₃₁FN₃O₄ (MH⁺): calcd, 432.22986. found, 432.22907.

### Example 251

The following compound was prepared consistent with the methods described herein.

### 4-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanoic Acid

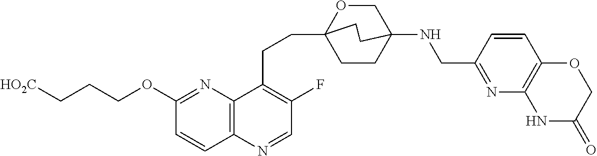 [SMILES: O=C(O)CCCOc1ccc2ncc(F)c(CCC34CCC(NCc5ccc6c(n5)NC(=O)CO6)(CC3)CO4)c2n1]

¹H NMR (DMSO-d₆) δ 1.49-1.97 (m, 10H), 2.03 (quintet, J=6.7 Hz, 2H), 2.40 (t, J=7.3 Hz, 2H), 3.02-3.14 (m, 2H), 3.67 (brs, 4H), 4.48 (t, J=6.7 Hz, 2H), 4.62 (s, 2H), 7.01-7.10 (m, 1H), 7.20 (d, J=9.2 Hz, 1H), 7.28-7.38 (m, 1H), 8.26 (d, J=9.2 Hz, 1H), 8.74 (s, 1H), 11.20 (brs, 1H).

MS (ESI⁺) m/z: 566 (MH⁺).

HRMS (ESI⁺) for C₂₉H₃₃FN₅O₆ (MH⁺): calcd, 566.24149. found, 566.24097.

### Example 252

The following compound was prepared consistent with the methods described herein.

### 7-((1-(2-(3-Fluoro-6-methoxy-1,5-naphthyridin-4-yl)ethyl)-2-oxabicyclo[2.2.2]octan-4-ylamino)methyl)-5-methylpyrido[3,2-b]pyrazin-6(5H)-one Hydrochloride

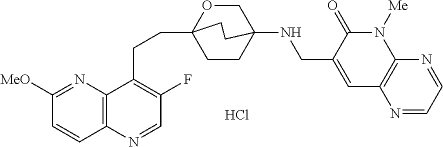 [SMILES: COc1ccc2ncc(F)c(CCC34CCC(NCc5cc6nccnc6n(C)c5=O)(CC3)CO4)c2n1 ; Cl]

¹H NMR (DMSO-d₆) δ 1.68-1.76 (m, 2H), 1.80-1.95 (m, 2H), 1.96-2.16 (m, 6H), 3.10-3.08 (m, 2H), 3.79 (s, 3H), 3.95 (brs, 2H), 4.05 (s, 3H), 4.16 (brs, 2H), 7.24 (d, J=9.2 Hz, 1H), 8.27 (d, J=9.2 Hz, 1H), 8.36 (s, 1H), 8.69 (d, J=1.8 Hz, 1H), 8.76 (s, 1H), 8.77 (d, J=2.4 Hz, 1H), 9.19 (brs, 2H).

MS (ESI⁺) m/z: 505 (MH⁺) (as free base).

HRMS (ESI⁺) for C₂₇H₃₀FN₆O₃ (MH⁺) (as free base): calcd, 505.23634. found 505.23567.

### Example 253

### 6-((1-(2-(6-Methoxypyrido[3,2-d]pyrimidin-4-yl)ethyl)-2-oxabicyclo[2.2.2]octan-4-ylamino)methyl)-2H-pyrido[3,2-b][1,4]oxazin-3(4H)-one Hydrochloride

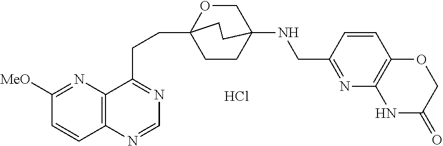 [SMILES: COc1ccc2ncnc(CCC34CCC(NCc5ccc6c(n5)NC(=O)CO6)(CC3)CO4)c2n1 ; Cl]

The title compound (95.3 mg) was prepared from 1-(2-(6-methoxypyrido[3,2-d]pyrimidin-4-yl)ethyl)-2-oxabicyclo[2.2.2]octan-4-amine (90.0 mg) and 3-oxo-3,4-dihydro-2H-pyrido[3,2-b][1,4]oxazine-6-carbaldehyde (50.2 mg) in the same manner as described for Step 3 of EXAMPLE 1.

¹H NMR (DMSO-d₆) δ 1.75-2.10 (m, 10H), 3.28-3.33 (m, 2H), 3.86 (brs, 2H), 4.05 (brs, 3H), 4.06-4.12 (m, 2H), 4.68 (s, 2H), 7.20 (d, J=7.9 Hz, 1H), 7.44 (d, J=7.9 Hz, 1H), 7.49 (d, J=9.2 Hz, 1H), 8.26 (d, J=9.2 Hz, 1H), 9.08 (s, 1H), 9.27 (brs, 2H), 11.31 (s, 1H)

MS (ESI⁺) m/z: 477 (MH⁺) (as free base).

HRMS (ESI⁺) for C₂₅H₂₉N₆O₄ (MH⁺) (as free base): calcd, 477.22503. found 477.22479.

### Preparation of Intermediates

### Step 1

### Preparation of 6-methoxypyrido[3,2-d]pyrimidin-4(3H)-one

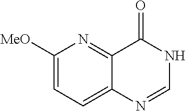 [SMILES: COc1ccc2nc[nH]c(=O)c2n1]

A mixture of 3-amino-6-methoxypicolinamide (2.14 g) and triethylorthoformate (64 mL) was stirred at 170° C. for 24 hours and concentrated in vacuo. Flash chromatography (silica, hexane:ethyl acetate=5:2) of the residue gave the title compound (1.38 g).

¹H NMR (DMSO-d₆) δ 3.95 (s, 3H), 7.26 (d, J=8.6 Hz, 1H), 7.98 (d, J=9.2 Hz, 1H), 8.04 (s, 1H), 12.48 (brs, 1H).

MS (ESI⁺) m/z: 178 (MH⁺).

HRMS (ESI⁺) calcd for C₈H₈N₃O₂ (MH⁺): 178.06165. found 178.06155.

### Step 2

### Preparation of 4-chloro-6-methoxypyrido[3,2-d]pyrimidine

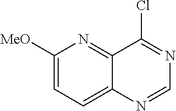 [SMILES: COc1ccc2ncnc(Cl)c2n1]

A mixture of 6-methoxypyrido[3,2-d]pyrimidin-4(3H)-one, thionyl chloride (18 mL) and N,N-dimethylformamide (5 drops) was stirred at 90° C. for 1 hour and concentrated in vacuo. Flash chromatography (silica, hexane:ethyl acetate=4:1) of the residue gave the title compound (1.57 g).

¹H NMR (DMSO-d₆) δ 4.08 (s, 3H), 7.60 (d, J=8.6 Hz, 1H), 8.35 (d, J=9.2 Hz, 1H), 9.03 (s, 1H).

MS (EI⁺) m/z: 195 (M⁺).

HRMS (EI⁺) for C₈H₆ClN₃O (M⁺): calcd, 195.0199. found 195.0221.

### Step 3

### Preparation of tert-butyl 1-(2-(6-Methoxypyrido[3,2-d]pyrimidin-4-yl)ethyl)-2-oxabicyclo[2.2.2]octan-4-ylcarbamate

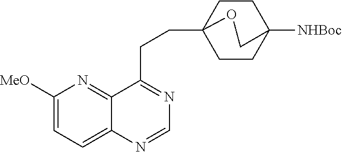 [SMILES: COc1ccc2ncnc(CCC34CCC(NC(=O)OC(C)(C)C)(CC3)CO4)c2n1]

To a solution of tert-butyl 1-vinyl-2-oxabicyclo[2.2.2]octan-4-ylcarbamate (1.90 g) in tetrahydrofuran was added a solution of 9-BBN (30 mL, 0.5M in tetrahydrofuran) at 4° C., the mixture was stirred at room temperature for 4 hours. A solution of potassium carbonate (10 mL, 2M) was added to the mixture, the mixture was stirred at room temperature for 30 minutes. To the mixture was added 4-chloro-6-methoxypyrido[3,2-d]pyrimidine (978 mg) and N,N-dimethylformamide (30 mL), the mixture was degassed and added triphenylphosphine (578 mg). The resulting mixture was stirred at 85° C. for 16 hours. After quenching the reaction by adding 10% citric acid solution (20 mL), the mixture was concentrated in vacuo. After dilution of the residue with ethyl acetate, the mixture was washed with brine. The organic extracts were dried over anhydrous sodium sulfate, filtered, and then concentrated in vacuo. Flash chromatography (silica, hexane:ethyl acetate=20:1) of the residue gave the title compound (1.77 g).

¹H NMR (DMSO-d₆) δ 1.34 (s, 9H), 1.64-1.98 (m, 10H), 3.24-3.32 (m, 2H), 3.73 (brs, 2H), 4.24 (s, 3H), 6.57 (brs, 1H), 7.47 (d, J=9.2 Hz, 1H), 8.24 (d, J=9.2 Hz, 1H), 9.06 (s, 1H).

MS (ESI⁺) m/z: 415 (MH⁺).

HRMS (ESI⁺) for C₂₂H₃₁N₄O₄ (MH⁺): calcd, 415.23453. found 415.23523.

### Step 4

### Preparation of 1-(2-(6-methoxypyrido[3,2-d]pyrimidin-4-yl)ethyl)-2-oxabicyclo[2.2.2]octan-4-amine

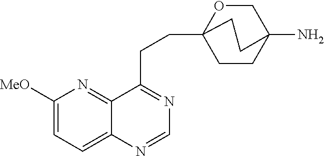 [SMILES: COc1ccc2ncnc(CCC34CCC(N)(CC3)CO4)c2n1]

To a solution of tert-butyl 1-(2-(6-methoxypyrido[3,2-d]pyrimidin-4-yl)ethyl)-2-oxabicyclo[2.2.2]octan-4-ylcarbamate (200 mg) in dichloromethane (2.4 mL) was added trifluoroacetic acid (2.2 mL) at 4° C., the mixture was stirred at room temperature for 2 hours and then concentrated in vacuo. Flash chromatography (silica, hexane:ethyl acetate=5:2) of the residue gave the title compound (151 mg).

¹H NMR (DMSO-d₆) δ 1.30 (brs, 1H), 1.48-1.62 (m, 4H), 1.62-1.72 (m, 2H), 1.75-1.85 (m, 4H), 3.26-3.32 (m, 2H), 3.41 (brs, 2H), 4.04 (s, 3H), 7.47 (d, J=8.6 Hz, 1H), 8.24 (d, J=9.2 Hz, 1H), 9.06 (s, 1H).

MS (ESI⁺) m/z: 315 (MH⁺).

HRMS (ESI⁺) for C₁₇H₂₃N₄O₂ (MH⁺): calcd, 318.18210. found 315.18211.

### Example 254

The following compound was prepared consistent with the methods described herein.

### 6-((1-(2-(6-((2-Aminocyclopropyl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

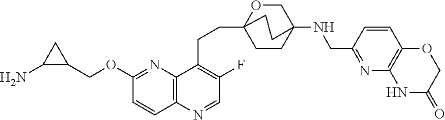 [SMILES: NC1CC1COc1ccc2ncc(F)c(CCC34CCC(NCc5ccc6c(n5)NC(=O)CO6)(CC3)CO4)c2n1]

¹H NMR (CDCl₃) δ 0.19-0.24 (m, 1H), 0.70-0.76 (m, 1H), 1.19-1.23 (m, 1H), 1.61-1.90 (m, 10H), 2.44-2.49 (m, 1H), 3.07-3.11 (m, 2H), 3.57 (s, 2H), 3.62 (s, 2H), 4.49-4.54 (m, 1H), 4.59 (s, 2H), 4.65-4.69 (m, 1H), 7.01 (d, J=7.9 Hz, 1H), 7.20 (d, J=9.2 Hz, 1H), 7.27 (d, J=7.9 Hz, 1H), 8.24 (d, J=9.2 Hz, 1H), 8.72 (s 1H), 11.1 (s, 1H).

[α]_{D}²⁸ −7.6 (c 0.1, MeOH).

### Example 255

### 4-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanenitrile

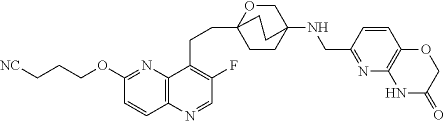 [SMILES: N#CCCCOc1ccc2ncc(F)c(CCC34CCC(NCc5ccc6c(n5)NC(=O)CO6)(CC3)CO4)c2n1]

¹H NMR (DMSO-d₆) δ 1.54-1.78 (m, 8H), 1.80-1.94 (m, 3H), 2.13 (quintet, J=6.7 Hz, 2H), 2.68 (t, J=7.3 Hz, 2H), 3.05-3.15 (m, 2H), 3.58 (s, 2H), 3.63 (d, J=4.3 Hz, 2H), 4.53 (t, J=6.1 Hz, 2H), 4.59 (s, 2H), 7.01 (d, J=7.9 Hz, 1H), 7.21 (d, J=9.2 Hz, 1H), 7.27 (d, J=7.9 Hz, 1H), 8.28 (d, J=9.2 Hz, 1H), 8.75 (s, 1H), 11.14 (s, 1H).

MS (ESI) m/z: 547 (MH⁺).

HRMS (ESI⁺) for C₂₉H₃₂FN₆O₄ (MH⁺): calcd, 547.24691. found, 547.24713.

### Preparation of Intermediates

### Step 1

### Preparation of tert-butyl 1-(2-(6-(3-cyanopropoxy)-3-fluoro-1,5-naphthyridin-4-yl)ethyl)-2-oxabicyclo[2.2.2]octan-4-ylcarbamate

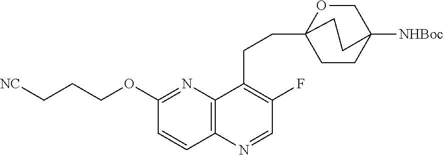 [SMILES: CC(C)(C)OC(=O)NC12CCC(CCc3c(F)cnc4ccc(OCCCC#N)nc34)(CC1)OC2]

The title compound 112 mg) was prepared from tert-butyl 1-(2-(3-fluoro-6-hydroxy-1,5-naphthyridin-4-yl)ethyl)-2-oxabicyclo[2.2.2]octan-4-ylcarbamate (125 mg) and 4-bromobutanenitrile (48.8 mg) in the same manner as described for Step 1 of EXAMPLE 32.

¹H NMR (CDCl₃) δ 1.44 (s, 9H), 1.67-1.81 (m, 4H), 1.82-1.92 (m, 2H), 1.97-2.18 (m, 4H), 2.19-2.28 (m, 2H), 2.61 (t, J=7.3 Hz, 2H), 3.13-3.22 (m, 2H), 3.97 (s, 2H), 4.29 (brs, 1H), 4.62 (t, J=6.1 Hz, 2H), 7.05 (d, J=9.2 Hz, 1H), 8.19 (d, J=9.2 Hz, 1H), 8.61 (s, 1H).

MS (ESI⁺) m/z: 485.3 (MH⁺).

HRMS (ESI⁺) for C₂₆H₃₄FN₄O₄ (MH⁺): calcd, 485.25641. found, 485.25715.

### Step 2

### Preparation of 4-(8-(2-(4-amino-2-oxabicyclo[2.2.2]octan-1-yl)ethyl)-7-fluoro-1,5-naphthyridin-2-yloxy)butanenitrile

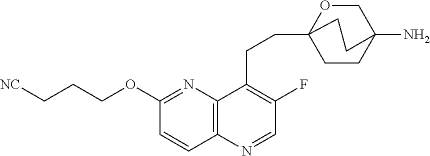 [SMILES: N#CCCCOc1ccc2ncc(F)c(CCC34CCC(N)(CC3)CO4)c2n1]

The title compound (73.8 mg) was prepared from tert-butyl 1-(2-(6-(3-cyanopropoxy)-3-fluoro-1,5-naphthyridin-4-yl)ethyl)-2-oxabicyclo[2.2.2]octan-4-ylcarbamate (105 mg) in the same manner as described for Step 2 of EXAMPLE 32.

¹H NMR (CDCl₃) δ 1.64-1.81 (m, 8H), 1.95-2.06 (m, 2H), 2.19-2.28 (m, 2H), 2.61 (t, J=7.3 Hz, 2H), 3.14-3.23 (m, 2H), 3.66 (s, 2H), 4.62 (t, J=6.1 Hz, 2H), 7.06 (d, J=9.2 Hz, 1H), 8.19 (d, J=8.6 Hz, 1H), 8.61 (s, 1H).

MS (ESI⁺) m/z: 385 (MH⁺).

HRMS (ESI⁺) for C₂₁H₂₆FN₄O₂ (MH⁺): calcd, 385.20398. found, 385.20316.

### Example 256

### Ethyl 4-(7-Cyano-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanoate

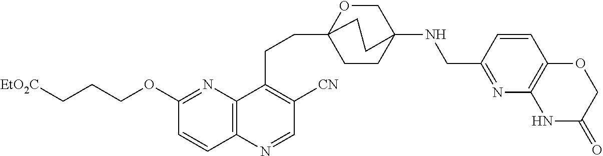 [SMILES: CCOC(=O)CCCOc1ccc2ncc(C#N)c(CCC34CCC(NCc5ccc6c(n5)NC(=O)CO6)(CC3)CO4)c2n1]

¹H NMR (DMSO-d₆) δ 1.15 (t, J=7.3 Hz, 3H), 1.55-1.79 (m, 8H), 1.80-1.95 (m, 3H), 2.07 (quintet, J=6.7 Hz, 2H), 2.46-2.52 (m, 2H), 3.23-3.33 (m, 2H), 3.59 (s, 2H), 3.63 (d, J=6.7 Hz, 2H), 4.05 (q, J=7.3 Hz, 2H), 4.49 (t, J=6.7 Hz, 2H), 4.59 (s, 2H), 7.01 (d, J=8.6 Hz, 1H), 7.28 (d, J=8.6 Hz, 1H), 7.39 (d, J=9.2 Hz, 1H), 8.32 (d, J=9.2 Hz, 1H), 8.97 (s, 1H), 11.15 (s, 1H).

MS (ESI⁺) m/z: 601 (MH⁺).

HRMS (ESI⁺) for C₃₂H₃₇N₆O₆ (MH⁺): calcd, 601.27746. found, 601.27661.

### Preparation of Intermediates

### Step 1

### Preparation of ethyl 4-(8-(2-(4-(tert-Butoxycarbonylamino)-2-oxabicyclo[2.2.2]octan-1-yl)ethyl)-7-cyano-1,5-naphthyridin-2-yloxy)butanoate

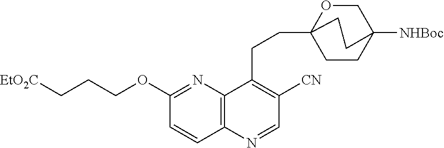 [SMILES: CCOC(=O)CCCOc1ccc2ncc(C#N)c(CCC34CCC(NC(=O)OC(C)(C)C)(CC3)CO4)c2n1]

The title compound (131 mg) was prepared from tert-butyl 1-(2-(3-cyano-6-hydroxy-1,5-naphthyridin-4-yl)ethyl)-2-oxabicyclo[2.2.2]octan-4-ylcarbamate (130 mg) and ethyl 4-bromobutanoate (65.7 mg) in the same manner as described for Step 1 of EXAMPLE 32.

¹H NMR (CDCl₃) δ 1.27 (t, J=7.3 Hz, 3H), 1.43 (s, 9H), 1.72-1.83 (m, 4H), 1.84-1.94 (m, 2H), 1.98-2.15 (m, 4H), 2.15-2.24 (m, 2H), 2.54 (t, J=7.3 Hz, 2H), 3.34-3.42 (m, 2H), 3.95 (s, 2H), 4.16 (q, J=7.3 Hz, 2H), 4.28 (brs, 1H), 4.55 (t, J=6.1 Hz, 2H), 7.19 (d, J=8.6 Hz, 1H), 8.20 (d, J=9.2 Hz, 1H), 8.80 (s, 1H).

MS (ESI⁺) m/z: 539 (MH⁺).

HRMS (ESI⁺) for C₂₉H₃₉N₄O₆ (MH⁺): calcd, 539.28696. found, 539.28641.

### Step 2

### Preparation of ethyl 4-(8-(2-(4-Amino-2-oxabicyclo[2.2.2]octan-1-yl)ethyl)-7-cyano-1,5-naphthyridin-2-yloxy)butanoate

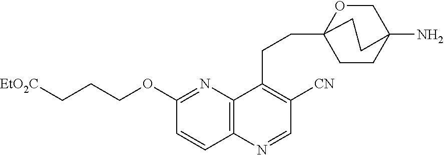 [SMILES: CCOC(=O)CCCOc1ccc2ncc(C#N)c(CCC34CCC(N)(CC3)CO4)c2n1]

The title compound (111 mg) was prepared from ethyl 4-(8-(2-(4-(tert-butoxycarbonylamino)-2-oxabicyclo[2.2.2]octan-1-yl)ethyl)-7-cyano-1,5-naphthyridin-2-yloxy)butanoate (127 mg) in the same manner as described for Step 2 of EXAMPLE 32.

¹H NMR (CDCl₃) δ 1.26 (t, J=7.3 Hz, 3H), 1.63-1.84 (m, 8H), 1.96-2.07 (m, 2H), 2.19 (dt, J=7.3, 6.1 Hz, 2H), 2.54 (t, J=7.3 Hz, 2H), 3.35-3.43 (m, 2H), 3.65 (s, 2H), 4.16 (q, J=7.3 Hz, 2H), 4.53 (t, J=6.1 Hz, 2H), 7.19 (d, J=8.6 Hz, 1H), 8.20 (d, J=9.2 Hz, 1H), 8.81 (s, 1H).

MS (ESI⁺) m/z: 439 (MH⁺).

HRMS (ESI⁺) for C₂₄H₃₁FN₄O₄ (MH⁺): calcd, 439.23453. found, 439.23385.

### Example 257

### 4-(7-Cyano-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanoic Acid

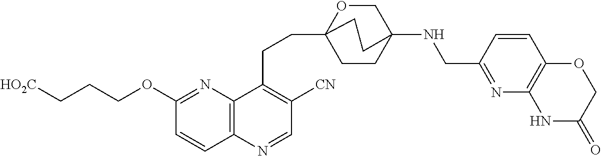 [SMILES: N#Cc1cnc2ccc(OCCCC(=O)O)nc2c1CCC12CCC(NCc3ccc4c(n3)NC(=O)CO4)(CC1)CO2]

¹H NMR (DMSO-d₆) δ 1.57-1.81 (m, 8H), 1.82-1.98 (m, 3H), 2.04 (quintet, J=6.7 Hz, 2H), 2.41 (t, J=7.3 Hz, 2H), 3.19-3.39 (m, 2H), 3.62 (brs, 4H), 4.49 (t, J=6.7 Hz, 2H), 4.60 (s, 2H), 6.98-7.08 (m, 1H), 7.24-7.35 (m, 1H), 7.40 (d, J=9.2 Hz, 1H), 8.33 (d, J=9.2 Hz, 1H), 8.97 (s, 1H), 11.17 (brs, 1H).

MS (ESI⁺) m/z: 573 (MH⁺).

HRMS (ESI⁺) for C₃₀H₃₃N₆O₆ (MH⁺): calcd, 573.24616. found, 573.24600.

### Example 258

### 6-((1-(2-(6-(((2S,3S)-3-Aminooxetan-2-yl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

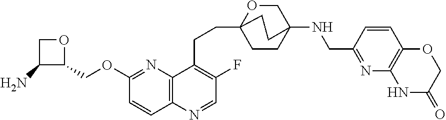 [SMILES: N[C@H]1CO[C@@H]1COc1ccc2ncc(F)c(CCC34CCC(NCc5ccc6c(n5)NC(=O)CO6)(CC3)CO4)c2n1]

The title compound (37.0 mg) was prepared from benzyl (2S,3S)-2-((7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)oxetan-3-ylcarbamate (75.0 mg) in the same manner as described for Step 4 of EXAMPLE 38.

¹H NMR (DMSO-d₆) δ 1.54-1.79 (m, 8H), 1.81-1.95 (m, 2H), 3.00-3.14 (m, 2H), 3.59 (s, 2H), 3.63 (s, 2H), 3.84 (dd, J=14.0, 6.7 Hz, 1H), 4.18 (t, J=6.7 Hz, 1H), 4.54 (dd, J=7.4, 6.1 Hz, 1H), 4.57-4.64 (m, 4H), 4.67-4.77 (m, 1H), 7.01 (d, J=8.6 Hz, 1H), 7.26 (d, J=8.6 Hz, 1H), 7.28 (d, J=8.0 Hz, 1H), 8.28 (d, J=9.2 Hz, 1H), 8.75 (s, 1H).

MS (ESI⁺) m/z: 565 (MH⁺).

HRMS (ESI⁺) for C₂₉H₃₄FN₆O₅ (MH⁺): calcd, 565.25747. found, 565.25780.

### Preparation of Intermediates

### Step 1

### Preparation of (2S,3S)-2-azido-4-(benzyloxy)-3-hydroxybutyl 4-Methylbenzenesulfonate

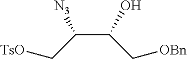 [SMILES: Cc1ccc(S(=O)(=O)OC[C@H](N=[N+]=[N-])[C@H](O)COCc2ccccc2)cc1]

The title compound (3.81 g) was prepared from (2S,3S)-2-azido-4-(benzyloxy)butane-1,3-diol (5.00 g) in the same manner as described for Step 1 of EXAMPLE 263.

¹H NMR (400 MHz, DMSO-d₆) δ 2.41 (s, 3H), 3.39-3.49 (m, 2H), 3.67 (dd, J=10.4, 4.9 Hz, 1H), 3.80 (ddd, J=8.6, 6.1, 3.0 Hz, 1H), 4.06 (dd, J=11.0, 8.6 Hz, 1H), 4.28 (dd, J=11.0, 3.0 Hz, 1H), 4.45 (s, 2H), 5.45 (d, J=5.5 Hz, 1H), 7.25-7.38 (m, 5H), 7.47 (d, J=7.9 Hz, 2H), 7.77 (d, J=7.9 Hz, 2H).

MS (ESI⁺) m/z: 409 (M+NH₄⁺).

HRMS (ESI⁺) for C₁₈H₂₅N₄O₅S (M+NH₄⁺): calcd, 409.15456.1280. found, 409.15447.

### Step 2

### Preparation of (2S,3S)-3-Azido-2-(benzyloxymethyl)oxetane

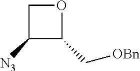 [SMILES: [N-]=[N+]=N[C@H]1CO[C@@H]1COCc1ccccc1]

The title compound (1.73 g) was prepared from (2S,3S)-2-azido-4-(benzyloxy)-3-hydroxybutyl 4-methylbenzenesulfonate (3.70 g) in the same manner as described for Step 2 of EXAMPLE 263.

¹H NMR (CDCl₃) δ 3.66 (ddd, J=14.1, 10.4, 2.4 Hz, 2H), 4.47-4.54 (m, 2H), 4.63 (dd, J=33.0, 11.6 Hz, 1H), 4.67-4.74 (m, 1H), 4.77-4.82 (m, 1H), 7.28-7.39 (m, 5H).

MS (EI⁺) m/z: 219 (M⁺).

HRMS (EI⁺) for C₁₁H₁₃N₃O₂ (M⁺): calcd, 219.10078. found, 219.10089.

### Step 3

### Preparation of benzyl (2S,3S)-2-(Hydroxymethyl)oxetan-3-ylcarbamate

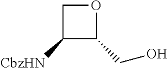 [SMILES: O=C(N[C@H]1CO[C@@H]1CO)OCc1ccccc1]

The title compound (144 mg) was prepared from (2S,3S)-3-azido-2-(benzyloxymethyl)oxetane (200 mg) in the same manner as described for Step 4 of EXAMPLE 263.

¹H NMR (CDCl₃) δ 2.36-2.47 (m, 1H), 3.68-3.86 (m, 2H), 4.39-4.49 (m, 1H), 4.62-4.76 (m, 3H), 5.11 (dd, J=14.1, 12.2 Hz, 2H), 5.21 (br s, 1H), 7.30-7.39 (m, 5H).

MS (ESI⁺) m/z: 238 (MH⁺).

HRMS (ESI⁺) for C₁₂H₁₆NO₄ (MH⁺): calcd, 238.10793. found, 238.10839.

### Step 4

### Preparation of benzyl (2S,3S)-2-(Bromomethyl)oxetan-3-ylcarbamate

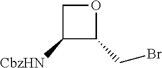 [SMILES: O=C(N[C@H]1CO[C@@H]1CBr)OCc1ccccc1]

The title compound (109 mg) was prepared from benzyl (2S,3S)-2-(hydroxymethyl)oxetan-3-ylcarbamate (140 mg) in the same manner as described for X.

¹H NMR (CDCl₃) δ 3.60 (d, J=4.3 Hz, 1H), 4.40 (t, J=6.7 Hz, 1H), 4.53-4.66 (m, 1H), 4.67-4.79 (m, 1H), 5.11 (dd, J=14.7, 12.8 Hz, 1H), 5.21 (brs, 1H), 7.30-7.43 (m, 5H).

MS (FI⁺) m/z: 299 (M⁺).

HRMS (FI⁺) for C₁₂H₁₄BrNO₃ (M⁺): calcd, 299.01571. found, 299.01502.

### Step 5

### Preparation of tert-butyl 1-(2-(6-(((2S,3S)-3-Benzyloxycarbonylaminooxetan-2-yl)methoxy)-3-fluoro-1,5-naphthyridin-4-yl)ethyl)-2-oxabicyclo[2.2.2]octan-4-ylcarbamate

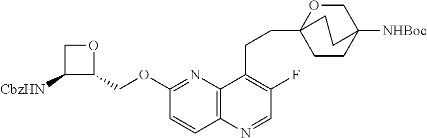 [SMILES: CC(C)(C)OC(=O)NC12CCC(CCc3c(F)cnc4ccc(OC[C@H]5OC[C@@H]5NC(=O)OCc5ccccc5)nc34)(CC1)OC2]

The title compound (167 mg) was prepared from tert-butyl 1-(2-(3-fluoro-6-hydroxy-1,5-naphthyridin-4-yl)ethyl)-2-oxabicyclo[2.2.2]octan-4-ylcarbamate (133 mg) and benzyl (2S,3S)-2-(bromomethyl)oxetan-3-ylcarbamate (105 mg) in the same manner as described for Step 1 of EXAMPLE 32.

¹H NMR (CDCl₃) δ 1.43 (s, 9H), 1.63-1.90 (m, 6H), 1.93-2.15 (m, 4H), 3.07-3.23 (m, 2H), 3.92-4.01 (m, 2H), 4.24-4.31 (m, 1H), 4.45-4.54 (m, 1H), 4.64-4.86 (m, 4H), 5.00-5.11 (m, 3H), 5.67 (brs, 1H), 7.14 (d, J=9.2 Hz, 1H), 7.29-7.40 (m, 5H), 8.19 (d, J=9.2 Hz, 1H), 8.60 (s, 1H).

MS (ESI⁺) m/z: 637 (MH⁺).

HRMS (ESI⁺) for C₃₄H₄₂FN₄O₇ (MH⁺): calcd, 637.30375. found, 637.30406.

### Step 6

### Preparation of benzyl (2S,3S)-2-((8-(2-(4-Amino-2-oxabicyclo[2.2.2]octan-1-yl)ethyl)-7-fluoro-1,5-naphthyridin-2-yloxy)methyl)oxetan-3-ylcarbamate

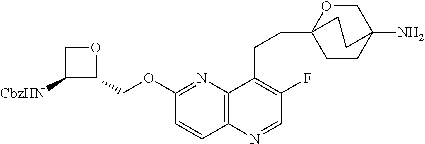 [SMILES: NC12CCC(CCc3c(F)cnc4ccc(OC[C@H]5OC[C@@H]5NC(=O)OCc5ccccc5)nc34)(CC1)OC2]

The title compound (105 mg) was prepared from tert-butyl 1-(2-(6-(((2S,3S)-3-benzyloxycarbonylaminooxetan-2-yl)methoxy)-3-fluoro-1,5-naphthyridin-4-yl)ethyl)-2-oxabicyclo[2.2.2]octan-4-ylcarbamate (160 mg) in the same manner as described for Step 2 of EXAMPLE 32.

¹H NMR (CDCl₃) δ 1.46-1.81 (m, 8H), 1.90-2.04 (m, 2H), 3.09-3.23 (m, 2H), 3.64 (s, 2H), 4.45-4.53 (m, 1H), 4.64-4.87 (m, 4H), 5.00-5.11 (m, 3H), 5.65 (brs, 1H), 7.14 (d, J=9.2 Hz, 1H), 7.28-7.40 (m, 5H), 8.19 (d, J=9.2 Hz, 1H), 8.61 (s, 1H).

MS (ESI⁺) m/z: 537 (MH⁺).

HRMS (ESI⁺) for C₂₉H₃₄FN₄O₅ (MH⁺): calcd, 537.25132. found, 537.25105.

### Step 7

### Preparation of benzyl (2S,3S)-2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)oxetan-3-ylcarbamate

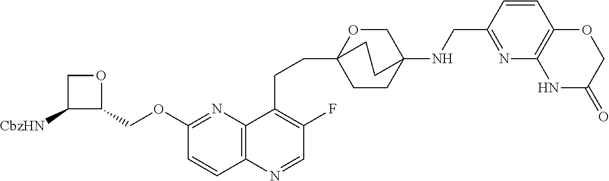 [SMILES: O=C1COc2ccc(CNC34CCC(CCc5c(F)cnc6ccc(OC[C@H]7OC[C@@H]7NC(=O)OCc7ccccc7)nc56)(CC3)OC4)nc2N1]

The title compound (78.3 mg) was prepared from benzyl (2S,3S)-2-((8-(2-(4-amino-2-oxabicyclo[2.2.2]octan-1-yl)ethyl)-7-fluoro-1,5-naphthyridin-2-yloxy)methyl)oxetan-3-ylcarbamate (103 mg) and 3-oxo-3,4-dihydro-2H-pyrido[3,2-b][1,4]oxazine-6-carbaldehyde (35.9 mg) in the same manner as described for Step 3 of EXAMPLE 1.

¹H NMR (CDCl₃) δ 1.66-1.83 (m, 8H), 1.95-2.06 (m, 2H), 3.08-3.25 (m, 2H), 3.74 (s, 2H), 3.77 (s, 2H), 4.43-4.54 (m, 1H), 4.63 (s, 2H), 4.65-4.79 (m, 2H), 4.84 (brs, 1H), 4.97-5.12 (m, 3H), 5.65 (brs, 1H), 6.93 (d, J=7.9 Hz, 1H), 7.14 (d, J=9.2 Hz, 1H), 7.19 (d, J=7.9 Hz, 1H), 7.28-7.38 (m, 5H), 8.20 (d, J=9.2 Hz, 1H), 8.61 (s, 1H).

MS (ESI⁺) m/z: 699 (MH⁺).

HRMS (ESI⁺) for C₃₇H₄₀FN₆O₇ (MH⁺): calcd, 699.29425. found, 699.29405.

### Example 259

### 4-(2-Hydroxy-2-(4-((3-oxo-3,4-dihydro-2H-pyrido[3,2-b][1,4]oxazin-6-yl)methylamino)-2-oxabicyclo[2.2.2]octan-1-yl)ethyl)-6-oxo-5,6,7,8-tetrahydro-1,5-naphthyridine-3-carbonitrile

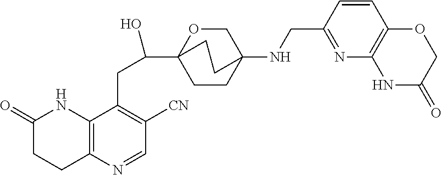 [SMILES: N#Cc1cnc2c(c1CC(O)C13CCC(NCc4ccc5c(n4)NC(=O)CO5)(CC1)CO3)NC(=O)CC2]

The title compound (64.0 mg) was prepared from 4-(2-(4-amino-2-oxabicyclo[2.2.2]octan-1-yl)-2-hydroxyethyl)-6-oxo-5,6,7,8-tetrahydro-1,5-naphthyridine-3-carbonitrile (53.0 mg) and 3-oxo-3,4-dihydro-2H-pyrido[3,2-b][1,4]oxazine-6-carbaldehyde (28.0 mg) in the same manner as described for Step 3 of EXAMPLE 1.

¹H NMR (CDCl₃) δ 1.48-2.00 (m, 7H), 2.12-2.23 (m, 1H), 2.60-2.80 (m, 3H), 2.92-2.98 (m, 1H), 3.12-3.33 (m, 3H), 3.48 (q, J=6.7 Hz, 1H), 3.66 (d, J=9.8 Hz, 1H), 3.75 (s, 2H), 3.79 (s, 2H), 4.64 (s, 2H), 6.94 (m, J=7.9 Hz, 1H), 7.20 (d, J=7.9 Hz, 1H), 8.14 (s, 1H), 8.43 (s, 1H), 9.10 (s, 1H).

MS (ESI⁺) m/z: 505 (MH⁺).

HRMS (ESI⁺) for C₂₆H₂₉N₆O₅ (MH⁺): calcd, 505.21994. found, 505.21965.

### Preparation of Intermediates

### Step 1

### Preparation of 4-bromo-6-hydroxy-1,5-naphthyridine-3-carboxylic Acid

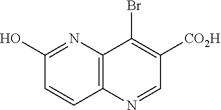 [SMILES: O=C(O)c1cnc2ccc(O)nc2c1Br]

4-Bromo-6-methoxy-1,5-naphthyridine-3-carboxylic acid (9.95 g) was added to a solution of hydrobromic acid in acetic acid (100 mL, 5.1M) under cooling with ice bath, the mixture was stirred at room temperature for 17 hours. The mixture was adjusted to pH 1-2 by addition of 30% sodium hydroxide solution under cooling with ice, then concentrated in vacuo. Treatment of the residue with water gave the title compound (9.33 g).

¹H NMR (DMSO-d₆) δ 6.71 (d, J=9.8 Hz, 1H), 7.84 (d, J=9.8 Hz, 1H), 8.25 (s, 1H), 10.57 (brs, 1H).

MS (FAB⁺) m/z: 291 (M+Na⁺).

HRMS (FAB⁺) for C₉H₅BrN₂NaO₃ (M+Na⁺): calcd, 290.9381. found, 290.9409.

### Step 2

### Preparation of benzyl 6-(Benzyloxy)-4-bromo-1,5-naphthyridine-3-carboxylate

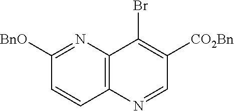 [SMILES: O=C(OCc1ccccc1)c1cnc2ccc(OCc3ccccc3)nc2c1Br]

To a suspension of 4-bromo-6-hydroxy-1,5-naphthyridine-3-carboxylic acid (8.80 g) and silver carbonate (18.1 g) was added benzylbromide (9.8 mL), the mixture was stirred at room temperature for 18 hours. After the insoluble materials were filtered off, the filtrate was concentrated in vacuo. Flash chromatography (silica, hexane:ethyl acetate=5:2) of the residue gave the title compound (10.6 g).

¹H NMR (CDCl₃) δ 5.49 (s, 2H), 5.65 (s, 2H), 7.25 (d, J=9.2 Hz, 1H), 7.30-7.44 (m, 6H), 7.49-7.53 (m, 2H), 7.57-7.62 (m, 2H), 8.22 (d, J=8.6 Hz, 1H), 8.97 (s, 1H).

MS (ESI⁺) m/z: 449 (MH⁺).

HRMS (ESI⁺) for C₂₃H₁₈BrN₂O₃ (MH⁺): calcd, 449.05008. found, 449.05011.

### Step 3

### Preparation of benzyl 6-(Benzyloxy)-4-methyl-1,5-naphthyridine-3-carboxylate

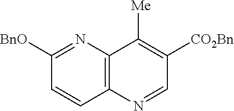 [SMILES: Cc1c(C(=O)OCc2ccccc2)cnc2ccc(OCc3ccccc3)nc12]

The title compound (391 mg) was prepared from benzyl 6-(benzyloxy)-4-bromo-1,5-naphthyridine-3-carboxylate (556 mg) in the same manner as described for R.

¹H NMR (CDCl₃) δ 3.03 (s, 3H), 5.45 (s, 2H), 5.58 (s, 2H), 7.23 (d, J=9.8 Hz, 1H), 7.32-7.45 (m, 6H), 7.48-7.55 (m, 4H), 8.21 (d, J=8.6 Hz, 1H), 9.18 (s, 1H).

MS (ESI⁺) m/z: 385 (MH⁺).

HRMS (ESI⁺) for C₂₄H₂₁N₂O₃ (MH⁺): calcd, 385.15522. found, 385.15471.

### Step 4

### Preparation of 6-(benzyloxy)-4-methyl-1,5-naphthyridine-3-carboxylic Acid

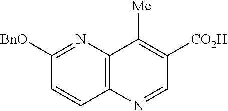 [SMILES: Cc1c(C(=O)O)cnc2ccc(OCc3ccccc3)nc12]

To a suspension of benzyl 6-(benzyloxy)-4-methyl-1,5-naphthyridine-3-carboxylate (4.02 g) in dimethyl sulfoxide (52 mL) was added water (52 mL) and potassium hydroxide (2.05 g), the mixture was stirred at 50° C. for 4 hours. The mixture was adjusted to pH 3-4 by addition of 10% citric acid solution, the resulting precipitates were collected by filtration. Flash chromatography (silica, chloroform:methanol=10:1) of the crude product gave the title compound (2.22 g).

¹H NMR (DMSO-d₆) δ 2.94 (s, 3H), 5.57 (s, 2H), 7.30-7.35 (m, 1H), 7.36-7.41 (m, 2H), 7.39 (d, J=9.2 Hz, 1H), 7.52-7.56 (m, 2H), 8.30 (d, J=8.6 Hz, 1H), 9.03 (s, 1H), 13.56 (brs, 1H).

MS (ESI⁺) m/z: 503.2 (MH⁺).

HRMS (ESI⁺) for C₂₆H₂₇N₆O₅ (MH⁺): calcd, 503.20429. found, 503.20498.

### Step 5

### Preparation of 6-(benzyloxy)-4-methyl-1,5-naphthyridine-3-carboxamide

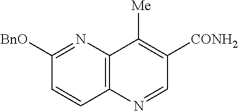 [SMILES: Cc1c(C(N)=O)cnc2ccc(OCc3ccccc3)nc12]

To a suspension of 6-(benzyloxy)-4-methyl-1,5-naphthyridine-3-carboxylic acid (2.67 g) and pyridine (2.2 mL) was added di-tert-butyl dicarbonate (5.95 g), the mixture was stirred at room temperature for 1 hour. Ammonium carbonate was added to the mixture, the mixture was stirred at the same temperature for 16 hours and concentrated in vacuo. After dilution of the residue with water, the resulting precipitates were collected by filtration. Flash chromatography (silica, chloroform:methanol=20:1) of the crude product gave the title compound (2.28 g).

¹H NMR (CDCl₃) δ 2.90 (s, 3H), 5.45 (s, 2H), 5.58 (s, 2H), 5.87 (brs, 2H), 7.22 (d, J=9.2 Hz, 1H), 7.31-7.42 (m, 6H), 7.50-7.55 (m, 2H), 8.20 (d, J=9.2 Hz, 1H), 8.81 (s, 1H).

MS (ESI⁺) m/z: 294 (MH⁺).

HRMS (ESI⁺) for C₁₇H₁₆N₃O₂ (MH⁺): calcd, 294.12425. found, 294.12405.

### Step 6

### Preparation of 6-(benzyloxy)-4-methyl-1,5-naphthyridine-3-carbonitrile

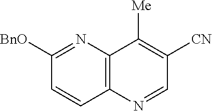 [SMILES: Cc1c(C#N)cnc2ccc(OCc3ccccc3)nc12]

To a suspension of 6-(benzyloxy)-4-methyl-1,5-naphthyridine-3-carboxamide (2.27 g) and triethylamine (5.8 mL) in dichloromethane (7.7 mL) was added trifluoroacetic anhydride (2.8 mL) under cooling with ice, the mixture was stirred at room temperature for 2 hours. After dilution of the mixture with dichloromethane, the mixture was washed with water. The organic extracts were washed with brine, dried over anhydrous sodium sulfate, filtered, and then concentrated in vacuo. Flash chromatography (silica, hexane:ethyl acetate=4:1) of the residue gave the title compound (1.82 g).

¹H NMR (CDCl₃) δ 2.96 (s, 3H), 5.57 (s, 2H), 7.29 (d, J=9.2 Hz, 1H), 7.34-7.43 (m, 3H), 7.50-7.54 (m, 2H), 8.23 (d, J=9.2 Hz, 1H), 8.83 (s, 1H).

MS (ESI⁺) m/z: 275 (MH⁺).

HRMS (ESI⁺) for C₁₇H₁₃N₃O₁ (MH⁺): calcd, 275.10586. found, 275.10645.

### Step 7

### Preparation of tert-butyl 1-(2-(6-(Benzyloxy)-3-cyano-1,5-naphthyridin-4-yl)-1-hydroxyethyl)-2-oxabicyclo[2.2.2]octan-4-ylcarbamate

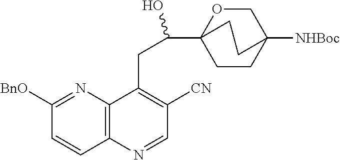 [SMILES: CC(C)(C)OC(=O)NC12CCC(C(O)Cc3c(C#N)cnc4ccc(OCc5ccccc5)nc34)(CC1)OC2]

The title compound (1.08 g) was prepared from 6-(benzyloxy)-4-methyl-1,5-naphthyridine-3-carbonitrile (900 mg) and Intermediate F (835 mg) in the same manner as described for Step 1 of EXAMPLE 20.

¹H NMR (CDCl₃) δ 1.43 (s, 9H), 1.72-1.94 (m, 4H), 2.10-2.24 (m, 4H), 2.59 (d, J=6.7

Hz, 1H), 3.41 (dd, J=12.2, 10.4 Hz, 1H), 3.49 (d, J=3.7 Hz, 1H), 3.60 (d, J=12.2, 2.4 Hz, 1H), 3.77-3.84 (m, 1H), 3.98-4.25 (m, 2H), 4.31 (brs, 1H), 5.54 (s, 2H), 7.29 (d, J=9.2 Hz, 1H), 7.34-7.43 (m, 3H), 7.45-7.50 (m, 2H), 8.26 (d, J=8.6 Hz, 1H), 8.86 (s, 1H).

MS (ESI⁺) m/z: 531 (MH⁺).

HRMS (ESI⁺) for C₃₀H₃₅N₄O₅ (MH⁺): calcd, 531.26074. found, 531.26046.

### Step 8

### Preparation of tert-butyl 1-(2-(3-Cyano-6-oxo-5,6,7,8-tetrahydro-1,5-naphthyridin-4-yl)-1-hydroxyethyl)-2-oxabicyclo[2.2.2]octan-4-ylcarbamate

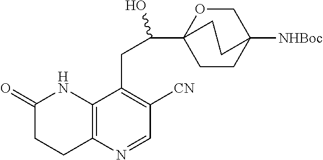 [SMILES: CC(C)(C)OC(=O)NC12CCC(C(O)Cc3c(C#N)cnc4c3NC(=O)CC4)(CC1)OC2]

A suspension of tert-butyl 1-(2-(6-(benzyloxy)-3-cyano-1,5-naphthyridin-4-yl)-1-hydroxyethyl)-2-oxabicyclo[2.2.2]octan-4-ylcarbamate (150 mg) and 10% Pd—C (45 mg) in N,N-dimethylformamide (3.0 mL) was stirred at room temperature for 4 hours under H₂ atmosphere (1 kg/cm²). After the insoluble materials were filtered off. Flash chromatography (silica, chloroform:methanol=15:1) of the residue gave the title compound (95.4 mg).

¹H NMR (CDCl₃) δ 1.43 (s, 9H), 1.80-2.00 (m, 5H), 2.08-2.24 (m, 3H), 2.60-2.80 (m, 2H), 3.12 (s, 1H), 3.14-3.32 (m, 2H), 3.64 (d, J=10.4 Hz, 1H), 3.98 (dd, J=8.0, 3.0 Hz, 1H), 4.02-4.10 (m, 1H), 4.33 (brs, 1H), 8.43 (s, 1H), 9.07 (s, 1H).

MS (ESI⁺) m/z: 443 (MH⁺).

HRMS (ESI⁺) for C₂₃H₃₁N₄O₅ (MH⁺): calcd, 443.22944. found, 443.22984.

### Step 9

### Preparation of 4-(2-(4-amino-2-oxabicyclo[2.2.2]octan-1-yl)-2-hydroxyethyl)-6-oxo-5,6,7,8-tetrahydro-1,5-naphthyridine-3-carbonitrile

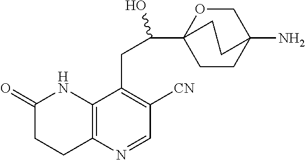 [SMILES: N#Cc1cnc2c(c1CC(O)C13CCC(N)(CC1)CO3)NC(=O)CC2]

The title compound (55.6 mg) was prepared from tert-butyl 1-(2-(3-cyano-6-oxo-5,6,7,8-tetrahydro-1,5-naphthyridin-4-yl)-1-hydroxyethyl)-2-oxabicyclo[2.2.2]octan-4-ylcarbamate (89.8 mg) in the same manner as described for Step 2 of EXAMPLE 1.

¹H NMR (CDCl₃) δ 1.62-1.84 (m, 6H), 1.90-2.00 (m, 2H), 2.08-2.20 (m, 1H), 2.60-2.80 (m, 3H), 2.93 (d, J=1.2 Hz, 1H), 3.10-3.32 (m, 1H), 3.62-3.70 (m, 3H), 8.43 (s, 1H), 9.09 (s, 1H).

MS (ESI⁺) m/z: 343 (MH⁺).

HRMS (ESI⁺) for C₁₈H₂₃N₄O₃ (MH⁺): calcd, 343.17701. found, 343.17766.

### Example 260

### 6-((1-(2-(7-(2-Hydroxyethoxy)-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one

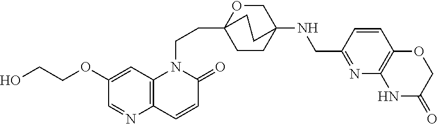 [SMILES: O=C1COc2ccc(CNC34CCC(CCn5c(=O)ccc6ncc(OCCO)cc65)(CC3)OC4)nc2N1]

The title compound (38.0 mg) was prepared from 1-(2-(4-amino-2-oxabicyclo[2.2.2]octan-1-yl)ethyl)-7-(2-hydroxyethoxy)-1,5-naphthyridin-2(1H)-one (54.0 mg) and 3-oxo-3,4-dihydro-2H-pyrido[3,2-b][1,4]oxazine-6-carbaldehyde (28.1 mg) in the same manner as described for Step 3 of EXAMPLE 1.

¹H NMR (DMSO-d₆) δ 1.56-1.74 (m, 8H), 1.80-1.98 (m, 3H), 3.35-3.40 (m, 1H), 3.63 (s, 2H), 3.65 (s, 2H), 3.79 (q, J=4.9 Hz, 2H), 4.19 (t, J=4.9 Hz, 2H), 4.22-4.25 (m, 2H), 4.59 (s, 2H), 5.00 (t, J=5.5 Hz, 1H), 6.63 (d, J=9.8 Hz, 1H), 7.01 (d, J=8.0 Hz, 1H), 7.28 (d, J=8.6 Hz, 1H), 7.42 (d, J=2.4 Hz, 1H), 7.84 (d, J=9.8 Hz, 1H), 8.27 (d, J=2.4 Hz, 1H), 11.15 (s, 1H).

MS (ESI⁺) m/z: 522 (MH⁺).

HRMS (ESI⁺) for C₂₇H₃₂N₅O₆ (MH⁺): calcd, 522.23526. found, 522.23489.

### Preparation of Intermediates

### Step 1

### Preparation of tert-butyl 1-(2-(7-(Benzyloxy)-2-oxo-1,5-naphthyridin-1(2H)-yl)ethyl)-2-oxabicyclo[2.2.2]octan-4-ylcarbamate

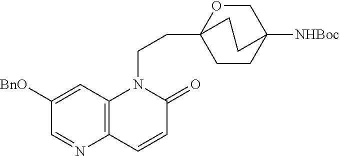 [SMILES: CC(C)(C)OC(=O)NC12CCC(CCn3c(=O)ccc4ncc(OCc5ccccc5)cc43)(CC1)OC2]

A suspension of 7-(benzyloxy)-1,5-naphthyridin-2(1H)-one (100 mg), 18-crown-6 (105 mg) and sodium carbonate (63.0 mg) in dioxane was stirred at room temperature for 55 minutes. tert-Butyl 1-(2-iodoethyl)-2-oxabicyclo[2.2.2]octan-4-ylcarbamate (166 mg) was added to the mixture. The resulting mixture was stirred at 125° C. for 32 hours. After dilution of the mixture with ethyl acetate, the mixture was washed with water. The organic extracts were washed with brine, dried over anhydrous sodium sulfate, filtered, and then concentrated in vacuo. Flash chromatography (silica, hexane:ethyl acetate=3:1) of the residue gave the title compound (139 mg).

¹H NMR (CDCl₃) δ 1.43 (s, 9H), 1.68-1.88 (m, 6H), 1.94-2.05 (m, 2H), 2.08-2.20 (m, 2H), 4.07 (s, 2H), 4.26-4.34 (m, 3H), 5.22 (s, 2H), 6.71 (d, J=9.8 Hz, 1H), 7.35-7.45 (m, 3H), 7.47-7.53 (m, 2H), 7.56 (d, J=2.4 Hz, 1H), 7.82 (d, J=9.8 Hz, 1H), 8.33 (d, J=2.4 Hz, 1H).

MS (ESI⁺) m/z: 506 (MH⁺).

HRMS (ESI⁺) for C₂₉H₃₆N₃O₅ (MH⁺): calcd, 506.26550. found, 506.26466.

### Step 2

### Preparation of tert-butyl 1-(2-(7-Hydroxy-2-oxo-1,5-naphthyridin-1(2H)-yl)ethyl)-2-oxabicyclo[2.2.2]octan-4-ylcarbamate

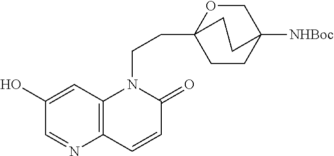 [SMILES: CC(C)(C)OC(=O)NC12CCC(CCn3c(=O)ccc4ncc(O)cc43)(CC1)OC2]

A suspension of tert-butyl 1-(2-(7-(benzyloxy)-2-oxo-1,5-naphthyridin-1(2H)-yl)ethyl)-2-oxabicyclo[2.2.2]octan-4-ylcarbamate (260 mg) and 10% Pd—C (40 mg) in dichloromethane (2.0 mL) and methanol (5.2 mL) was stirred at room temperature for 3 hours under H₂ atmosphere (1 kg/cm²). After the insoluble materials were filtered off, the filtrate was concentrated in vacuo to give the title compound (166 mg).

¹H NMR (DMSO-d₆) δ 1.36 (s, 9H), 1.52-1.58 (m, 2H), 1.64-1.74 (m, 2H), 1.76-2.00 (m, 3H), 3.82 (s, 2H), 4.07-4.15 (m, 2H), 6.56 (d, J=9.8 Hz, 1H), 6.61 (s, 1H), 7.13 (d, J=1.8 Hz, 1H), 7.79 (d, J=9.8 Hz, 1H), 8.12 (d, J=1.8 Hz, 1H), 10.83 (s, 1H).

MS (ESI⁺) m/z: 416 (MH⁺).

HRMS (ESI⁺) for C₂₂H₃₀N₃O₅ (MH⁺): calcd, 416.21855. found, 416.21801.

### Step 3

### Preparation of tert-butyl 1-(2-(2-Oxo-7-(2-(tetrahydro-2H-pyran-2-yloxy)ethoxy)-1,5-naphthyridin-1(2H)-yl)ethyl)-2-oxabicyclo[2.2.2]octan-4-ylcarbamate

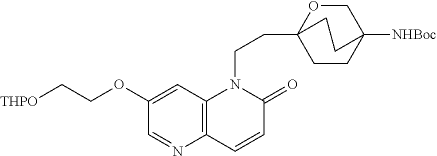

The title compound (96.4 mg) was prepared from tert-butyl 1-(2-(7-hydroxy-2-oxo-1,5-naphthyridin-1(2H)-yl)ethyl)-2-oxabicyclo[2.2.2]octan-4-ylcarbamate (75.0 mg) and 2-(2-bromoethoxy)tetrahydro-2H-pyran (50 uL) in the same manner as described for Step 1 of EXAMPLE 32.

¹H NMR (CDCl₃) δ 1.43 (s, 9H), 1.59-1.86 (m, 12H), 1.94-2.03 (m, 2H), 2.08-2.20 (m, 2H), 3.51-3.60 (m, 1H), 3.84-3.96 (m, 2H), 4.04 (s, 2H), 4.10-4.18 (m, 1H), 4.26-4.36 (m, 5H), 4.71-4.75 (m, 1H), 6.71 (d, J=9.8 Hz, 1H), 7.53 (d, J=1.8 Hz, 1H), 7.82 (d, J=9.8 Hz, 1H), 8.30 (d, J=2.4 Hz, 1H).

MS (ESI⁺) m/z: 544 (MH⁺).

HRMS (ESI⁺) for C₂₉H₄₂N₃O₇ (MH⁺): calcd, 544.30227. found, 544.30294.

### Step 4

### Preparation of 1-(2-(4-amino-2-oxabicyclo[2.2.2]octan-1-yl)ethyl)-7-(2-hydroxyethoxy)-1,5-naphthyridin-2(1H)-one

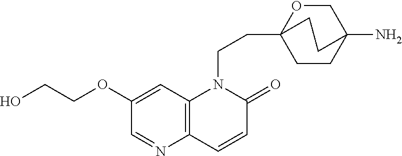 [SMILES: NC12CCC(CCn3c(=O)ccc4ncc(OCCO)cc43)(CC1)OC2]

The title compound (58.6 mg) was prepared from tert-butyl 1-(2-(2-oxo-7-(2-(tetrahydro-2H-pyran-2-yloxy)ethoxy)-1,5-naphthyridin-1(2H)-yl)ethyl)-2-oxabicyclo[2.2.2]octan-4-ylcarbamate (90.0 mg) in the same manner as described for Step 2 of EXAMPLE 32.

¹H NMR (DMSO-d₆) δ 1.50-1.72 (m, 8H), 1.78-1.90 (m, 2H), 3.54 (s, 2H), 3.76-3.82 (m, 2H), 4.17-4.24 (m, 4H), 4.96-5.04 (m, 1H), 6.63 (d, J=9.8 Hz, 1H), 7.42 (d, J=2.4 Hz, 1H), 7.84 (d, J=9.8 Hz, 1H), 8.27 (d, J=2.4 Hz, 1H).

MS (ESI⁺) m/z: 360 (MH⁺).

HRMS (ESI⁺) for C₁₉H₂₆N₃O₄ (MH⁺): calcd, 360.19233. found, 360.19221.

### Example 261

### 6-((1-(2-(7-(3-Hydroxypropoxy)-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one

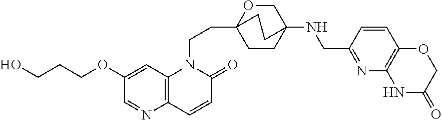 [SMILES: O=C1COc2ccc(CNC34CCC(CCn5c(=O)ccc6ncc(OCCCO)cc65)(CC3)OC4)nc2N1]

The title compound (72.7 mg) was prepared from 1-(2-(4-amino-2-oxabicyclo[2.2.2]octan-1-yl)ethyl)-7-(3-hydroxypropoxy)-1,5-naphthyridin-2(1H)-one (59.0 mg) and 3-oxo-3,4-dihydro-2H-pyrido[3,2-b][1,4]oxazine-6-carbaldehyde (30.0 mg) in the same manner as described for Step 3 of EXAMPLE 1.

¹H NMR (DMSO-d₆) δ 1.56-1.74 (m, 8H), 1.80-1.98 (m, 4H), 3.56-3.68 (m, 6H), 4.18-4.28 (m, 4H), 4.59 (s, 2H), 4.61 (t, J=5.5 Hz, 1H), 6.63 (d, J=9.8 Hz, 1H), 7.02 (d, J=7.9 Hz, 1H), 7.28 (d, J=7.9 Hz, 1H), 7.43 (d, J=2.4 Hz, 1H), 7.84 (d, J=9.8 Hz, 1H), 8.26 (d, J=2.4 Hz, 1H), 11.15 (s, 1H).

MS (ESI⁺) m/z: 536 (MH⁺).

HRMS (ESI⁺) for C₂₈H₃₄N₅O₆ (MH⁺): calcd, 536.25091. found, 536.25012.

### Preparation of Intermediates

### Step 1

### Preparation of tert-butyl 1-(2-(2-Oxo-7-(3-(Tetrahydro-2H-pyran-2-yloxy)propoxy)-1,5-naphthyridin-1(2H)-yl)ethyl)-2-oxabicyclo[2.2.2]octan-4-ylcarbamate

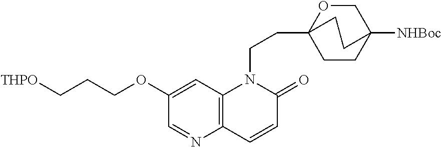

The title compound (96.4 mg) was prepared from tert-butyl 1-(2-(7-hydroxy-2-oxo-1,5-naphthyridin-1(2H)-yl)ethyl)-2-oxabicyclo[2.2.2]octan-4-ylcarbamate (75.0 mg) and 2-(3-bromopropoxy)tetrahydro-2H-pyran (61 uL) in the same manner as described for Step 1 of EXAMPLE 32.

¹H NMR (CDCl₃) δ 1.44 (s, 9H), 1.50-1.60 (m, 4H), 1.68-1.92 (m, 8H), 1.93-2.04 (m, 2H), 2.06-2.22 (m, 4H), 3.47-3.55 (m, 1H), 3.58-3.66 (m, 1H), 3.82-3.91 (m, 1H), 3.94-4.08 (m, 3H), 4.24 (dd, J=6.7, 6.1 Hz, 1H), 4.28-4.38 (m, 3H), 4.58-4.64 (m, 1H), 6.71 (d, J=9.8 Hz, 1H), 7.52 (d, J=2.4 Hz, 1H), 7.82 (d, J=9.8 Hz, 1H), 8.26 (d, J=2.4 Hz, 1H),

MS (ESI⁺) m/z: 558 (MH⁺).

HRMS (ESI⁺) for C₃₀H₄₄N₃O₇ (MH⁺): calcd, 558.31792. found, 558.31750.

### Step 2

### Preparation of 1-(2-(4-amino-2-oxabicyclo[2.2.2]octan-1-yl)ethyl)-7-(3-hydroxypropoxy)-1,5-naphthyridin-2(1H)-one

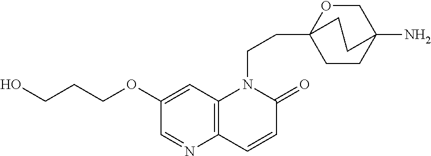 [SMILES: NC12CCC(CCn3c(=O)ccc4ncc(OCCCO)cc43)(CC1)OC2]

The title compound (62.1 mg) was prepared from tert-butyl 1-(2-(2-Oxo-7-(3-(Tetrahydro-2H-pyran-2-yloxy)propoxy)-1,5-naphthyridin-1(2H)-yl)ethyl)-2-oxabicyclo[2.2.2]octan-4-ylcarbamate (91.0 mg) in the same manner as described for Step 2 of EXAMPLE 32.

¹H NMR (DMSO-d₆) δ 1.50-1.75 (m, 8H), 1.78-1.98 (m, 4H), 3.52-3.62 (m, 4H), 4.18-4.26 (m, 4H), 4.61 (brs, 1H), 6.63 (d, J=9.2 Hz, 1H), 7.43 (d, J=1.8 Hz, 1H), 7.84 (d, J=9.8 Hz, 1H), 8.25 (d, J=1.8 Hz, 1H).

MS (ESI⁺) m/z: 374 (MH⁺).

HRMS (ESI⁺) for C₂₀H₂₈N₃O₄ (MH⁺): calcd, 374.20798. found, 374.20788.

### Example 262

### Methyl 5-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)pentanoate

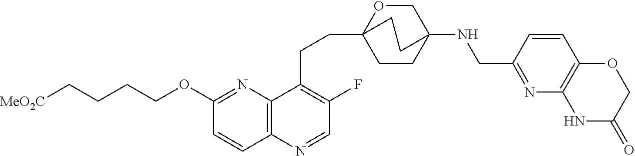 [SMILES: COC(=O)CCCCOc1ccc2ncc(F)c(CCC34CCC(NCc5ccc6c(n5)NC(=O)CO6)(CC3)CO4)c2n1]

The title compound (98.1 mg) was prepared from methyl 5-(8-(2-(4-amino-2-oxabicyclo[2.2.2]octan-1-yl)ethyl)-7-fluoro-1,5-naphthyridin-2-yloxy)pentanoate (90.0 mg) and 3-oxo-3,4-dihydro-2H-pyrido[3,2-b][1,4]oxazine-6-carbaldehyde (39.0 mg) in the same manner as described for Step 3 of EXAMPLE 1.

¹H NMR (DMSO-d₆) δ 1.56-1.93 (m, 14H), 2.39 (t, J=7.3 Hz, 2H), 3.04-3.13 (m, 2H), 3.57 (s, 3H), 3.58 (s, 2H), 3.62 (d, J=4.9 Hz, 2H), 4.47 (t, J=6.7 Hz, 2H), 4.59 (s, 2H), 7.01 (d, J=8.6 Hz, 1H), 7.20 (d, J=8.6 Hz, 1H), 7.28 (d, J=8.0 Hz, 1H), 8.25 (d, J=8.6 Hz, 1H), 8.73 (s, 1H), 11.15 (s, 1H).

MS (ESI⁺) m/z: 594 (MH⁺).

HRMS (ESI⁺) for C₃₁H₃₇FN₅O₆ (MH⁺): calcd, 594.27279. found, 594.27338.

### Preparation of Intermediates

### Step 1

### Preparation of ethyl 5-(8-(2-(4-(tert-Butoxycarbonylamino)-2-oxabicyclo[2.2.2]octan-1-yl)ethyl)-7-fluoro-1,5-naphthyridin-2-yloxy)pentanoate

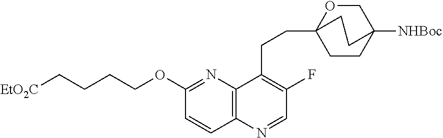 [SMILES: CCOC(=O)CCCCOc1ccc2ncc(F)c(CCC34CCC(NC(=O)OC(C)(C)C)(CC3)CO4)c2n1]

The title compound (165 mg) was prepared from tert-butyl 1-(2-(3-fluoro-6-hydroxy-1,5-naphthyridin-4-yl)ethyl)-2-oxabicyclo[2.2.2]octan-4-ylcarbamate (140 mg) and ethyl 5-bromopentanoate (77.1 mg) in the same manner as described for Step 1 of EXAMPLE 32.

¹H NMR (CDCl₃) δ 1.26 (t, J=7.3 Hz, 3H), 1.43 (s, 9H), 1.69-1.94 (m, 10H), 1.98-2.15 (m, 4H), 2.41 (dd, J=7.3, 6.7 Hz, 2H), 3.12-3.20 (m, 2H), 3.95 (s, 2H), 4.14 (q, J=7.3 Hz, 2H), 4.28 (s, 1H), 4.49 (dd, J=6.7, 5.5 Hz, 2H), 7.03 (d, J=8.6 Hz, 1H), 8.15 (d, J=9.2 Hz, 1H), 8.58 (s, 1H).

MS (ESI⁺) m/z: 546 (MH⁺).

HRMS (ESI⁺) for C₂₉H₄₁FN₃O₆ (MH⁺): calcd, 546.29794. found, 546.29710.

### Step 2

### Preparation of methyl 5-(8-(2-(4-Amino-2-oxabicyclo[2.2.2]octan-1-yl)ethyl)-7-fluoro-1,5-naphthyridin-2-yloxy)pentanoate

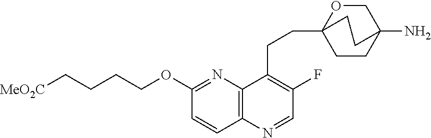 [SMILES: COC(=O)CCCCOc1ccc2ncc(F)c(CCC34CCC(N)(CC3)CO4)c2n1]

The title compound (76.9 mg) was prepared from methyl 5-(8-(2-(4-(tert-butoxycarbonylamino)-2-oxabicyclo[2.2.2]octan-1-yl)ethyl)-7-fluoro-1,5-naphthyridin-2-yloxy)pentanoate (160 mg) in the same manner as described for Step 2 of EXAMPLE 32.

¹H NMR (CDCl₃) δ: 1.60-1.93 (m, 12H), 1.94-2.06 (m, 2H), 2.43 (t, J=7.3 Hz, 2H), 3.14-3.22 (m, 2H), 3.65 (s, 2H), 3.69 (s, 3H), 4.50 (t, J=6.1 Hz, 2H), 7.03 (d, J=8.6 Hz, 1H), 8.15 (d, J=9.2 Hz, 1H), 8.58 (s, 1H).

MS (ESI⁺) m/z: 432 (MH⁺).

HRMS (ESI⁺) for C₂₃H₃₁FN₃O₄ (MH⁺): calcd, 432.22986. found, 432.22969.

### Example 263

### 2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylic Acid Hydrochloride

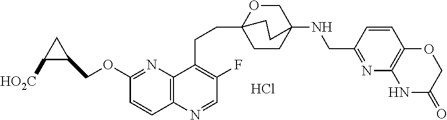 [SMILES: O=C1COc2ccc(CNC34CCC(CCc5c(F)cnc6ccc(OC[C@@H]7C[C@@H]7C(=O)O)nc56)(CC3)OC4)nc2N1 ; Cl]

¹H NMR (DMSO-d₆) δ 0.95-1.06 (m, 1H), 1.13-1.21 (m, 1H), 1.63-1.73 (m, 2H), 1.77-1.89 (m, 3H), 1.90-2.13 (m, 7H), 3.04-3.15 (m, 2H), 3.92 (s, 2H), 4.10 (s, 2H), 4.50 (dd, J=11.6, 9.2 Hz, 1H), 4.69 (s, 2H), 4.77 (dd, J=11.6, 6.1 Hz, 1H), 7.21 (d, J=9.2 Hz, 2H), 7.45 (d, J=7.9 Hz, 1H), 8.26 (d, J=9.2 Hz, 1H), 8.75 (s, 1H), 9.28 (s, 2H), 11.32 (s, 1H), 12.22 (brs, 1H).

MS (ESI) m/z: 578 (MH⁺) (as free base).

HRMS (ESI⁺) for C₃₀H₃₃FN₅O₆ (MH⁺) (as free base): calcd, 578.24149. found, 578.24173.

### Example 264

### 2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylic Acid Hydrochloride

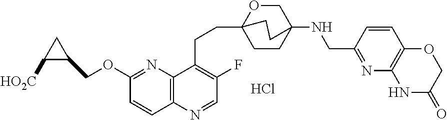 [SMILES: O=C1COc2ccc(CNC34CCC(CCc5c(F)cnc6ccc(OC[C@@H]7C[C@@H]7C(=O)O)nc56)(CC3)OC4)nc2N1 ; Cl]

¹H NMR (DMSO-d₆) δ 0.95-1.06 (m, 1H), 1.10-1.28 (m, 1H), 1.57-2.13 (m, 12H), 3.00-3.15 (m, 2H), 3.88 (brs, 2H), 4.09 (brs, 2H), 4.50 (dd, J=11.6, 9.2 Hz, 1H), 4.67 (s, 2H), 4.77 (dd, J=11.6, 6.1 Hz, 1H), 7.10-7.23 (m, 2H), 7.21 (d, J=9.2 Hz, 1H), 7.35-7.51 (m, 1H), 8.26 (d, J=9.2 Hz, 1H), 8.75 (s, 1H), 9.20 (s, 2H), 11.29 (s, 1H).

MS (ESI⁺) m/z: 578 (MH⁺) (as free base).

HRMS (ESI⁺) for C₃₀H₃₃FN₅O₆ (MH⁺) (as free base): calcd, 578.24149. found, 578.24220.

### Example 265

### 5-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)ₚ entanoic Acid

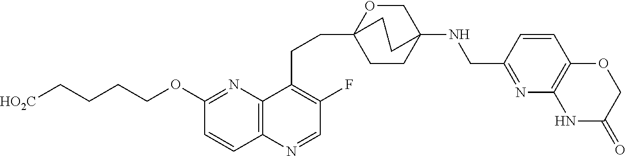 [SMILES: O=C(O)CCCCOc1ccc2ncc(F)c(CCC34CCC(NCc5ccc6c(n5)NC(=O)CO6)(CC3)CO4)c2n1]

¹H NMR (DMSO-d₆) δ 1.42-2.15 (m, 14H), 2.30 (t, J=7.3 Hz, 2H), 2.97-3.17 (m, 2H), 3.45-4.24 (m, 4H), 4.48 (t, J=6.7 Hz, 2H), 4.64 (brs, 2H), 6.89-7.15 (m, 1H), 7.20 (d, J=8.6 Hz, 1H), 7.26-7.51 (m, 1H), 8.25 (d, J=8.6 Hz, 1H), 8.74 (s, 1H), 11.22 (brs, 1H).

MS (ESI⁺) m/z: 580 (MH⁺).

HRMS (ESI⁺) for C₃₀H₃₅N₅FO₆ (MH⁺): calcd, 580.25714. found, 580.25716.

### Example 266

### Methyl 7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-2-carboxylate

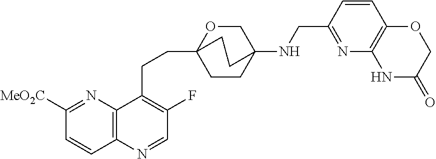 [SMILES: COC(=O)c1ccc2ncc(F)c(CCC34CCC(NCc5ccc6c(n5)NC(=O)CO6)(CC3)CO4)c2n1]

The title compound (63.8 mg) was prepared from methyl 8-(2-(4-amino-2-oxabicyclo[2.2.2]octan-1-yl)ethyl)-7-fluoro-1,5-naphthyridine-2-carboxylate (270 mg) and 3-oxo-3,4-dihydro-2H-pyrido[3,2-b][1,4]oxazine-6-carbaldehyde (109 mg) in the same manner as described for Step 3 of EXAMPLE 1.

¹H NMR (DMSO-d₆) δ 1.58-1.76 (m, 8H), 1.80-1.98 (m, 2H), 3.20-3.28 (m, 2H), 3.52 (s, 2H), 3.62 (s, 2H), 3.97 (s, 3H), 4.59 (s, 2H), 7.00 (d, J=8.6 Hz, 1H), 7.27 (d, J=8.0 Hz, 1H), 8.30 (d, J=8.6 Hz, 1H), 8.61 (d, J=8.6 Hz, 1H), 9.10 (s, 1H), 11.14 (s, 1H).

MS (ESI⁺) m/z: 522 (MH⁺).

HRMS (ESI⁺) for C₂₇H₂₉FN₅O₅ (MH⁺): calcd, 522.21527. found, 522.21519.

### Preparation of Intermediates

### Step 1

### Preparation of methyl 8-(2-(4-(tert-Butoxycarbonylamino)-2-oxabicyclo[2.2.2]octan-1-yl)ethyl)-7-fluoro-1,5-naphthyridine-2-carboxylate

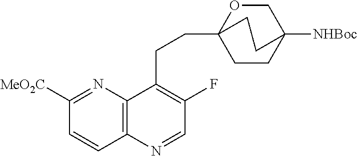 [SMILES: COC(=O)c1ccc2ncc(F)c(CCC34CCC(NC(=O)OC(C)(C)C)(CC3)CO4)c2n1]

CO gas was bubbled through a suspension of 8-(2-(4-(tert-butoxycarbonylamino)-2-oxabicyclo[2.2.2]octan-1-yl)ethyl)-7-fluoro-1,5-naphthyridin-2-yl trifluoromethanesulfonate (600 mg), triethylamine (0.32 mL), triphenylphosphine (18.0 mg) and palladium acetate (8.40 mg) in N,N-dimethylformamide (2.4 mL) and methanol (1.1 mL) for 2 minutes. The mixture was stirred at room temperature for 1.25 hours and at 60° C. for 3 hours. After dilution of the mixture with chloroform, the mixture was washed with brine. The organic extracts were dried over anhydrous sodium sulfate, filtered, and then concentrated in vacuo. Flash chromatography (silica, hexane:ethyl acetate=2:1) of the residue gave the title compound (297 mg).

¹H NMR (CDCl₃) δ 1.43 (s, 9H), 1.78-1.94 (m, 6H), 2.04-2.15 (m, 4H), 3.32-3.40 (m, 2H), 3.92 (s, 2H), 4.06 (s, 3H), 4.27 (brs, 1H), 8.33 (d, J=8.6 Hz, 1H), 8.49 (d, J=8.6 Hz, 1H), 8.88 (s, 1H).

MS (ESI⁺) m/z: 460 (MH⁺).

HRMS (ESI⁺) for C₂₄H₃₁FN₃O₅ (MH⁺): calcd, 460.22477. found, 460.22456.

### Step 2

### Preparation of methyl 8-(2-(4-Amino-2-oxabicyclo[2.2.2]octan-1-yl)ethyl)-7-fluoro-1,5-naphthyridine-2-carboxylate

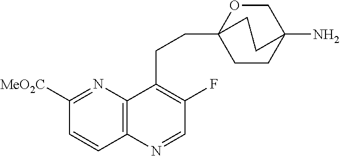 [SMILES: COC(=O)c1ccc2ncc(F)c(CCC34CCC(N)(CC3)CO4)c2n1]

The title compound (229 mg) was prepared from methyl 8-(2-(4-(tert-butoxycarbonylamino)-2-oxabicyclo[2.2.2]octan-1-yl)ethyl)-7-fluoro-1,5-naphthyridine-2-carboxylate (281 mg) in the same manner as described for Step 2 of EXAMPLE 32.

¹H NMR (DMSO-d₆) δ: 1.48-1.75 (m, 8H), 1.80-1.92 (m, 2H), 3.21-3.54 (m, 4H), 3.97 (s, 3H), 8.30 (d, J=8.6 Hz, 1H), 8.60 (d, J=9.2 Hz, 1H), 9.09 (s, 1H).

MS (ESI⁺) m/z: 360 (MH⁺).

HRMS (ESI⁺) for C₁₉H₂₃FN₃O₃ (MH⁺): calcd, 360.17234. found, 360.17316.

### Example 267

The following compound was prepared consistent with the methods described herein.

### 7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-2-carboxylic Acid

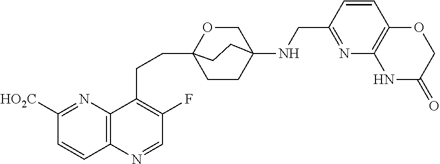 [SMILES: O=C1COc2ccc(CNC34CCC(CCc5c(F)cnc6ccc(C(=O)O)nc56)(CC3)OC4)nc2N1]

¹H NMR (DMSO-d₆) δ 1.58-1.78 (m, 8H), 1.85-1.97 (m, 2H), 3.19-3.40 (m, 2H), 3.55 (s, 2H), 3.65 (s, 2H), 4.59 (s, 2H), 7.01 (d, J=7.9 Hz, 1H), 7.28 (d, J=7.9 Hz, 1H), 8.25 (d, J=9.2 Hz, 1H), 8.54 (d, J=9.2 Hz, 1H), 9.05 (s, 1H), 11.16 (s, 1H).

MS (ESI⁺) m/z: 508 (MH⁺).

HRMS (ESI⁺) for C₂₆H₂₇FN₅O₅ (MH⁺): calcd, 508.19962. found, 508.19904.

### Example 268

### 6-((1-(2-(6-(((2R,3S)-3-Aminooxetan-2-yl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

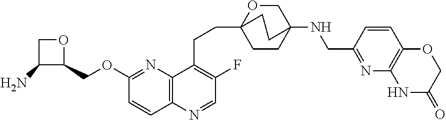 [SMILES: N[C@H]1CO[C@H]1COc1ccc2ncc(F)c(CCC34CCC(NCc5ccc6c(n5)NC(=O)CO6)(CC3)CO4)c2n1]

The title compound (23.4 mg) was prepared from benzyl (2R,3S)-2-((7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)oxetan-3-ylcarbamate (45.0 mg) in the same manner as described for Step 4 of EXAMPLE 38.

¹H NMR (DMSO-d₆) δ 1.56-1.81 (m, 8H), 1.84-1.96 (m, 2H), 3.03-3.20 (m, 2H), 3.58 (s, 2H), 3.62 (s, 2H), 4.18 (dd, J=14.7, 7.4 Hz, 1H), 4.32 (t, J=6.7 Hz, 1H), 4.59 (s, 2H), 4.70 (dd, J=7.3, 6.1 Hz, 1H), 4.74-4.86 (m, 2H), 4.95-5.01 (m, 1H), 7.00 (d, J=8.0 Hz, 1H), 7.26 (d, J=9.2 Hz, 1H), 7.28 (d, J=8.0 Hz, 1H), 8.27 (d, J=8.6 Hz, 1H), 8.75 (s, 1H), 11.15 (s, 1H).

MS (ESI⁺) m/z: 565 (MH⁺).

HRMS (ESI⁺) for C₂₉H₃₄FN₆O₅ (MH⁺): calcd, 565.25747. found, 565.25796.

### Preparation of Intermediates

### Step 1

### Preparation of (2S,3S)-2-Azido-4-(benzyloxy)butane-1,3-diyl Bis(4-methylbenzenesulfonate)

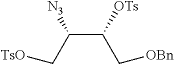 [SMILES: Cc1ccc(S(=O)(=O)OC[C@H](N=[N+]=[N-])[C@@H](COCc2ccccc2)OS(=O)(=O)c2ccc(C)cc2)cc1]

To a solution of (2S,3S)-2-azido-4-(benzyloxy)butane-1,3-diol (25.0 g), 4-(dimethylamino)pyridine (103 mg) and triethylamine (117 mL) in dichloromethane (215 mL) was added p-toluenesulfonyl chloride (80.4 g) at 0° C., the mixture was stirred at room temperature for 23 hours. After dilution of the mixture with dichloromethane, the mixture was washed with water. The organic extracts were washed with brine, dried over anhydrous sodium sulfate, filtered, and then concentrated in vacuo. Flash chromatography (silica, hexane:ethyl acetate=3:1) of the residue gave the title compound (57.6 g).

¹H NMR (CDCl₃) δ 2.45 (s, 6H), 3.60 (d, J=4.3 Hz, 2H), 3.91 (dd, J=10.4, 8.0 Hz, 1H), 4.02 (ddd, J=8.0, 6.1, 3.1 Hz, 1H), 4.20 (dd, J=10.4, 3.1 Hz, 1H), 4.41 (s, 2H), 4.51 (td, J=6.1, 4.3 Hz, 1H), 7.18-7.22 (m, 2H), 7.28-7.36 (m, 7H), 7.72-7.77 (m, 4H).

MS (ESI⁺) m/z: 563 (M+NH₄⁺).

HRMS (ESI⁺) for C₂₅H₃₁N₄O₇S₂ (M+NH₄⁺): calcd, 563.16341.1280. found, 563.16372.

### Step 2

### Preparation of (2S,3R)-2-Azido-4-(benzyloxy)butane-1,3-diyl Diacetate

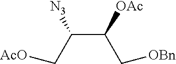 [SMILES: CC(=O)OC[C@H](N=[N+]=[N-])[C@H](COCc1ccccc1)OC(C)=O]

To a solution of (2S,3S)-2-azido-4-(benzyloxy)butane-1,3-diyl bis(4-methylbenzenesulfonate) (54.8 g) in toluene (2.2 L) was added cesium carbonate (193 g) and 18-crown-6 (53.1 g), the mixture was heated under reflux for 6 hours. The mixture was washed with water and brine. The organic extracts were dried over anhydrous sodium sulfate, filtered, and then concentrated in vacuo. Flash chromatography (silica, hexane:ethyl acetate=5:1) of the residue gave the title compound (13.9 g).

¹H NMR (CDCl₃) δ 2.09 (s, 3H), 2.11 (s, 3H), 3.62 (ddd, J=16.5, 10.4, 5.5 Hz, 1H), 3.95-4.02 (m, 1H), 4.13 (d, J=11.6, 8.0 Hz, 1H), 4.28 (dd, J=11.6, 4.3 Hz, 1H), 4.54 (dd, J=16.5, 12.2 Hz, 2H), 5.14 (dd, J=10.4, 4.9 Hz, 1H), 7.29-7.40 (m, 5H).

MS (ESI⁺) m/z: 322 (MH⁺).

HRMS (ESI⁺) for C₁₅H₂₀N₃O₅ (MH⁺): calcd, 322.14030. found, 322.14015.

### Step 3

### Preparation of (2S,3R)-2-Azido-4-(benzyloxy)butane-1,3-diol

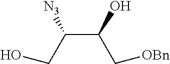 [SMILES: [N-]=[N+]=N[C@@H](CO)[C@@H](O)COCc1ccccc1]

To a solution of (2S,3R)-2-azido-4-(benzyloxy)butane-1,3-diyl diacetate (13.4 g) in methanol (140 mL) was added potassium carbonate (575 mg) under cooling with ice bath, the mixture was stirred at room temperature for 2 hours and concentrated in vacuo. After dilution of the residue with ethyl acetate, the mixture was washed with phosphate buffer solution (pH7) and brine. The organic extracts were dried over anhydrous sodium sulfate, filtered, and then concentrated in vacuo. Flash chromatography (silica, hexane:ethyl acetate=2:1) of the residue gave the title compound (7.28 g).

¹H NMR (DMSO-d₆) δ 3.34-3.39 (m, 1H), 3.40-3.47 (m, 3H), 3.54-3.66 (m, 2H), 3.76 (ddd, J=9.8, 6.1, 3.7 Hz, 1H), 4.48 (s, 2H), 4.95 (dd, J=6.1, 4.9 Hz, 1H), 5.11 (d, J=5.5 Hz, 1H), 7.24-7.37 (m, 5H).

MS (ESI⁺) m/z: 238 (MH⁺).

HRMS (ESI⁺) for C₁₁H₁₆N₃O₃ (MH⁺): calcd, 238.11917. found, 238.11917.

### Step 4

### Preparation of (2S,3R)-2-Azido-4-(benzyloxy)-3-hydroxybutyl 4-Methylbenzenesulfonate

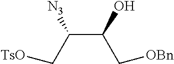 [SMILES: Cc1ccc(S(=O)(=O)OC[C@H](N=[N+]=[N-])[C@@H](O)COCc2ccccc2)cc1]

The title compound (5.00 g) was prepared from (2S,3R)-2-azido-4-(benzyloxy)butane-1,3-diol (5.00 g) in the same manner as described for Step 1 of EXAMPLE 263.

¹H NMR (CDCl₃) δ 2.45 (s, 3H), 3.53 (ddd, J=12.8, 9.2, 4.9 Hz, 2H), 3.76 (ddd, J=7.3, 4.9, 3.7 Hz, 1H), 3.88 (ddd, J=9.2, 4.9, 3.7 Hz, 1H), 4.26 (dd, J=10.4, 4.9 Hz, 1H), 4.53 (s, 2H), 7.28-7.39 (m, 7H), 7.81 (dd, J=8.6, 1.8 Hz, 1H).

MS (ESI⁺) m/z: 409 (M+NH₄⁺).

HRMS (FAB) for C₁₈H₂₅N₄O₅S (M+NH₄): calcd, 409.15456. found, 409.15405.

### Step 5

### Preparation of (2R,3S)-3-Azido-2-(benzyloxymethyl)oxetane

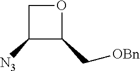 [SMILES: [N-]=[N+]=N[C@H]1CO[C@H]1COCc1ccccc1]

The title compound (1.39 g) was prepared from (2S,3R)-2-azido-4-(benzyloxy)-3-hydroxybutyl 4-methylbenzenesulfonate (4.98 g) in the same manner as described for Step 2 of EXAMPLE 263.

¹H NMR (CDCl₃) δ 3.75 (dd, J=10.4, 5.5 Hz, 1H), 3.89 (dd, J=10.4, 5.5 Hz, 1H), 4.48 (dd, J=7.9, 6.7 Hz, 1H), 4.61 (s, 2H), 4.73 (ddd, J=12.8, 7.3, 5.5 Hz, 1H), 4.82 (t, J=6.7 Hz, 1H), 5.02 (dd, J=12.8, 6.7 Hz, 1H), 7.27-7.38 (m, 5H).

MS (FI⁺) m/z: 219 (10.

HRMS (FI⁺) for C₁₁H₁₃N₃O₂ (M⁺): calcd, 219.10078. found, 219.10073.

### Step 6

### Preparation of benzyl (2R,3S)-2-(Hydroxymethyl)oxetan-3-ylcarbamate

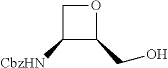 [SMILES: O=C(N[C@H]1CO[C@H]1CO)OCc1ccccc1]

The title compound (79.1 mg) was prepared from (2R,3S)-3-azido-2-(benzyloxymethyl)oxetane (200 mg) in the same manner as described for Step 4 of EXAMPLE 263.

¹H NMR (CDCl₃) δ 2.43 (dd, J=9.8, 2.4 Hz, 1H), 3.78 (dd, J=12.8, 9.8 Hz, 1H), 3.96 (dt, J=12.8, 3.1 Hz, 1H), 4.45 (t, J=6.7 Hz, 1H), 4.87-4.99 (m, 2H), 5.03-5.21 (m, 1H), 5.10 (dd, J=18.4, 12.2 Hz, 2H), 6.24-6.36 (m, 1H), 7.31-7.39 (m, 5H).

MS (FI⁺) m/z: 237 (10.

HRMS (FI⁺) for C₁₂H₁₅NO₄ (M⁺): calcd, 237.10011. found, 237.10094.

### Step 7

### Preparation of benzyl (2R,3S)-2-(Bromomethyl)oxetan-3-ylcarbamate

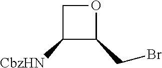 [SMILES: O=C(N[C@H]1CO[C@H]1CBr)OCc1ccccc1]

The title compound (46.5 mg) was prepared from benzyl (2R,3S)-2-(hydroxymethyl)oxetan-3-ylcarbamate (80 mg) in the same manner as described for X.

¹H NMR (CDCl₃) δ 3.51 (dd, J=11.0, 4.9 Hz, 1H), 3.60 (dd, J=11.6, 5.5 Hz, 1H), 4.39-4.54 (m, 2H), 4.81-4.94 (m, 1H), 4.99-5.10 (m, 1H), 5.12 (s, 2H), 5.42 (brs, 1H), 7.28-7.45 (m, 5H).

MS (ESI⁺) m/z: 300 (MH⁺).

HRMS (ESI⁺) for C₁₂H₁₅BrNO₃ (MH⁺): calcd, 300.02353. found, 300.02276.

### Step 8

### Preparation of tert-butyl 1-(2-(6-(((2R,3S)-3-Benzyloxycarbonylaminooxetan-2-yl)methoxy)-3-fluoro-1,5-naphthyridin-4-yl)ethyl)-2-oxabicyclo[2.2.2]octan-4-ylcarbamate

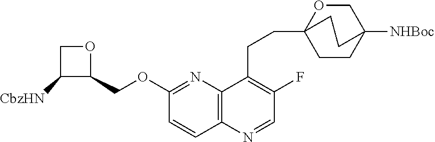 [SMILES: CC(C)(C)OC(=O)NC12CCC(CCc3c(F)cnc4ccc(OC[C@@H]5OC[C@@H]5NC(=O)OCc5ccccc5)nc34)(CC1)OC2]

The title compound (69.4 mg) was prepared from tert-butyl 1-(2-(3-fluoro-6-hydroxy-1,5-naphthyridin-4-yl)ethyl)-2-oxabicyclo[2.2.2]octan-4-ylcarbamate (56.9 mg) and benzyl (2R,3S)-2-(bromomethyl)oxetan-3-ylcarbamate (45.0 mg) in the same manner as described for Step 1 of EXAMPLE 32.

¹H NMR (CDCl₃) δ 1.43 (s, 9H), 1.62-1.90 (m, 6H), 1.92-2.16 (m, 4H), 3.08-3.25 (m, 2H), 3.91-3.99 (m, 2H), 4.25 (brs, 1H), 4.55-4.66 (m, 2H), 4.87-4.98 (m, 2H), 4.98-5.16 (m, 2H), 5.17-5.32 (m, 2H), 5.85-5.97 (m, 1H), 7.11 (d, J=9.2 Hz, 1H), 7.16-7.31 (m, 5H), 8.20 (d, J=8.6 Hz, 1H), 8.61 (s, 1H).

MS (ESI⁺) m/z: 637 (MH⁺).

HRMS (ESI⁺) for C₃₄H₄₂FN₄O₇ (MH⁺): calcd, 637.30375. found, 637.30277.

### Step 9

### Preparation of benzyl (2R,3S)-2-((8-(2-(4-Amino-2-oxabicyclo[2.2.2]octan-1-yl)ethyl)-7-fluoro-1,5-naphthyridin-2-yloxy)methyl)oxetan-3-ylcarbamate

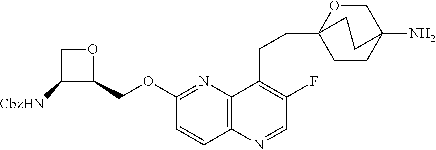 [SMILES: NC12CCC(CCc3c(F)cnc4ccc(OC[C@@H]5OC[C@@H]5NC(=O)OCc5ccccc5)nc34)(CC1)OC2]

The title compound (51.2 mg) was prepared from tert-butyl 1-(2-(6-(((2R,3S)-3-benzyloxycarbonylaminooxetan-2-yl)methoxy)-3-fluoro-1,5-naphthyridin-4-yl)ethyl)-2-oxabicyclo[2.2.2]octan-4-ylcarbamate (68.0 mg) in the same manner as described for Step 2 of EXAMPLE 32.

¹H NMR (CDCl₃) δ 1.65-1.86 (m, 7H), 1.89-2.09 (m, 3H), 3.09-3.24 (m, 2H), 3.59-3.66 (m, 2H), 4.62 (t, J=6.7 Hz, 2H), 4.87-4.99 (m, 2H), 5.01-5.15 (m, 2H), 5.17-5.32 (m, 2H), 5.92 (d, J=8.6 Hz, 1H), 7.12 (d, J=9.2 Hz, 1H), 7.17-7.36 (m, 5H), 8.21 (d, J=9.2 Hz, 1H), 8.61 (s, 1H).

MS (ESI⁺) m/z: 537 (MH⁺).

HRMS (ESI⁺) for C₂₉H₃₄FN₄O₅ (MH⁺): calcd, 537.25132. found, 537.25053.

### Step 10

### Preparation of benzyl (2R,3S)-2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)oxetan-3-ylcarbamate

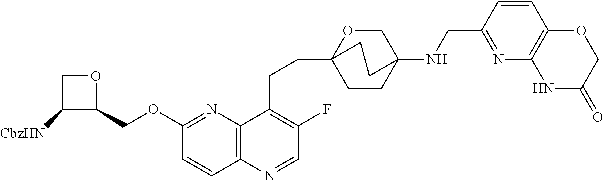 [SMILES: O=C1COc2ccc(CNC34CCC(CCc5c(F)cnc6ccc(OC[C@@H]7OC[C@@H]7NC(=O)OCc7ccccc7)nc56)(CC3)OC4)nc2N1]

The title compound (47.0 mg) was prepared from benzyl (2R,3S)-2-((8-(2-(4-amino-2-oxabicyclo[2.2.2]octan-1-yl)ethyl)-7-fluoro-1,5-naphthyridin-2-yloxy)methyl)oxetan-3-ylcarbamate (50.0 mg) and 3-oxo-3,4-dihydro-2H-pyrido[3,2-b][1,4]oxazine-6-carbaldehyde (17.4 mg) in the same manner as described for Step 3 of EXAMPLE 1.

¹H NMR (CDCl₃) δ 1.66-1.86 (m, 8H), 1.92-2.08 (m, 2H), 3.10-3.26 (m, 2H), 3.73 (s, 2H), 3.75 (s, 2H), 4.54-4.69 (m, 4H), 4.85-4.98 (m, 2H), 5.00-5.32 (m, 4H), 5.87 (d, J=9.2 Hz, 1H), 6.93 (d, J=8.0 Hz, 1H), 7.12 (d, J=8.6 Hz, 1H), 7.17-7.31 (m, 5H), 7.19 (d, J=8.6 Hz, 1H), 8.21 (d, J=8.6 Hz, 1H), 8.62 (s, 1H).

MS (ESI⁺) m/z: 699 (MH⁺).

HRMS (ESI⁺) for C₃₇H₄₀FN₆O₇ (MH⁺): calcd, 699.29425. found, 699.29485.

### Example 269

### Methyl 1-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylate

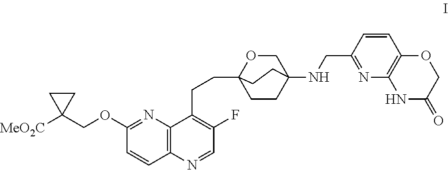 [SMILES: COC(=O)C1(COc2ccc3ncc(F)c(CCC45CCC(NCc6ccc7c(n6)NC(=O)CO7)(CC4)CO5)c3n2)CC1]

The title compound (127 mg) was prepared from methyl 1-((8-(2-(4-amino-2-oxabicyclo[2.2.2]octan-1-yl)ethyl)-7-fluoro-1,5-naphthyridin-2-yloxy)methyl)cyclopropanecarboxylate (108 mg) and 3-oxo-3,4-dihydro-2H-pyrido[3,2-b][1,4]oxazine-6-carbaldehyde (44.7 mg) in the same manner as described for Step 3 of EXAMPLE 1.

¹H NMR (CDCl₃) δ 1.10 (dd, J=7.4, 4.3 Hz, 2H), 1.42 (dd, J=7.4, 4.3 Hz, 2H), 1.71-1.86 (m, 8H), 1.98-2.08 (m, 2H), 3.13-3.21 (m, 2H), 3.73 (s, 3H), 3.76 (s, 2H), 3.77 (s, 2H), 4.63 (s, 2H), 4.67 (s, 2H), 6.94 (d, J=7.9 Hz, 1H), 7.07 (d, J=9.2 Hz, 1H), 7.20 (d, J=7.9 Hz, 1H), 8.04 (s, 1H), 8.16 (d, J=9.2 Hz, 1H), 8.59 (s, 1H).

MS (ESI⁺) m/z: 592 (MH⁺).

HRMS (ESI⁺) for C₃₁H₃₅FN₅O₆ (MH⁺): calcd, 592.25714. found, 592.25734.

### Preparation of Intermediates

### Step 1

### Preparation of methyl 1-((8-(2-(4-(tert-Butoxycarbonylamino)-2-oxabicyclo[2.2.2]octan-1-yl)ethyl)-7-fluoro-1,5-naphthyridin-2-yloxy)methyl)cyclopropanecarboxylate

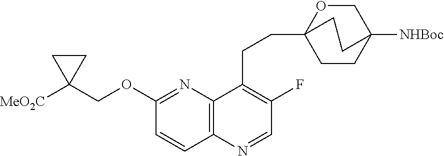 [SMILES: COC(=O)C1(COc2ccc3ncc(F)c(CCC45CCC(NC(=O)OC(C)(C)C)(CC4)CO5)c3n2)CC1]

The title compound (150 mg) was prepared from tert-butyl 1-(2-(3-fluoro-6-hydroxy-1,5-naphthyridin-4-yl)ethyl)-2-oxabicyclo[2.2.2]octan-4-ylcarbamate (115 mg) and methyl 1-(bromomethyl)cyclopropanecarboxylate (64.4 mg) in the same manner as described for Step 1 of EXAMPLE 32.

¹H NMR (CDCl₃) δ 1.10 (dd, J=6.8, 3.7 Hz, 2H), 1.40-1.46 (m, 11H), 1.70-1.83 (m, 4H), 1.83-1.95 (m, 2H), 1.97-2.17 (m, 4H), 3.09-3.21 (m, 2H), 3.73 (s, 3H), 3.96 (s, 2H), 4.29 (brs, 1H), 4.66 (s, 2H), 7.07 (d, J=9.2 Hz, 1H), 8.15 (d, J=9.2 Hz, 1H), 8.58 (s, 1H).

MS (ESI⁺) m/z: 530 (MH⁺).

HRMS (ESI⁺) for C₂₈H₃₇FN₃O₆ (MH⁺): calcd, 530.26664. found, 530.26727.

### Step 2

### Preparation of methyl 1-((8-(2-(4-Amino-2-oxabicyclo[2.2.2]octan-1-yl)ethyl)-7-fluoro-1,5-naphthyridin-2-yloxy)methyl)cyclopropanecarboxylate

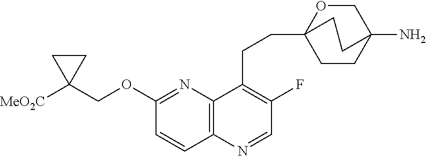 [SMILES: COC(=O)C1(COc2ccc3ncc(F)c(CCC45CCC(N)(CC4)CO5)c3n2)CC1]

The title compound (120 mg) was prepared from methyl 1-((8-(2-(4-(tert-butoxycarbonylamino)-2-oxabicyclo[2.2.2]octan-1-yl)ethyl)-7-fluoro-1,5-naphthyridin-2-yloxy)methyl)cyclopropanecarboxylate (148 mg) in the same manner as described for Step 2 of EXAMPLE 32.

¹H NMR (400 MHz, DMSO-d₆) δ: 1.13 (dd, J=6.7, 3.7 Hz, 2H), 1.27 (dd, J=6.8, 3.7 Hz, 2H), 1.46-1.72 (m, 8H), 1.76-1.88 (m, 2H), 3.03-3.11 (m, 2H), 3.45 (s, 2H), 3.62 (s, 3H), 4.62 (s, 2H), 7.23 (d, J=9.2 Hz, 1H), 8.24 (d, J=9.2 Hz, 1H), 8.73 (s, 1H).

MS (ESI⁺) m/z: 430 (MH⁺).

HRMS (ESI⁺) for C₂₃H₂₉FN₃O₄ (MH⁺): calcd, 430.21421. found, 430.21395.

### Example 270

The following compound was prepared consistent with the methods described herein.

### 1-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylic Acid

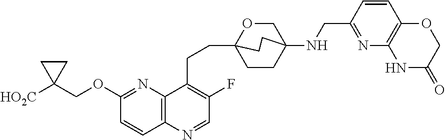 [SMILES: O=C1COc2ccc(CNC34CCC(CCc5c(F)cnc6ccc(OCC7(C(=O)O)CC7)nc56)(CC3)OC4)nc2N1]

¹H NMR (DMSO-d₆) δ 1.06 (dd, J=6.7, 3.7 Hz, 2H), 1.24 (dd, J=6.1, 3.7 Hz, 2H), 1.60-2.00 (m, 10H), 3.04-3.13 (m, 2H), 3.50-4.10 (m, 4H), 4.59 (s, 2H), 4.64 (s, 2H), 7.02-7.18 (m, 1H), 7.24 (d, J=9.2 Hz, 1H), 7.30-7.45 (m, 1H), 8.25 (d, J=9.2 Hz, 1H), 8.74 (s, 1H), 11.23 (brs, 1H).

MS (ESI⁺) m/z: 578 (MH⁺).

HRMS (ESI⁺) for C₃₀H₃₃FN₅O₆ (MH⁺): calcd, 578.24149. found, 578.24187.

### Example 271

The following compound was prepared consistent with the methods described herein.

### Methyl 2-((6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridin-3-yloxy)methyl)cyclopropanecarboxylate

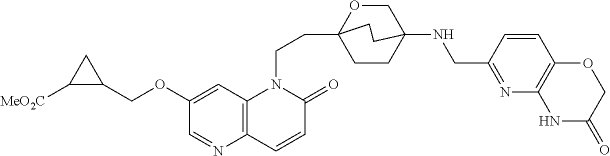 [SMILES: COC(=O)C1CC1COc1cnc2ccc(=O)n(CCC34CCC(NCc5ccc6c(n5)NC(=O)CO6)(CC3)CO4)c2c1]

¹H NMR (DMSO-d₆) δ 1.02-1.09 (m, 1H), 1.23-1.28 (m, 1H), 1.58-1.71 (m, 8H), 1.84-1.98 (m, 4H), 3.56 (s, 3H), 3.64 (s, 4H), 4.12-4.23 (m, 3H), 4.49 (dd, J=11.0, 5.5 Hz, 1H), 4.59 (s, 2H), 6.63 (d, J=9.8 Hz, 1H), 7.02 (d, J=7.9 Hz, 1H), 7.28 (d, J=7.9 Hz, 1H), 7.41 (d, J=1.8 Hz, 1H), 7.83 (d, J=9.8 Hz, 1H), 8.23 (d, J=1.8 Hz, 1H), 11.14 (s, 1H).

MS (ESI⁺) m/z: 590 (MH⁺).

HRMS (ESI⁺) for C₃₁H₃₆N₅O₇ (MH⁺): calcd, 590.26147. found, 590.26168.

### Example 272

The following compound was prepared consistent with the methods described herein.

### Methyl 2-((6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridin-3-yloxy)methyl)cyclopropanecarboxylate

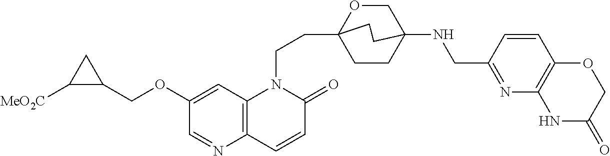 [SMILES: COC(=O)C1CC1COc1cnc2ccc(=O)n(CCC34CCC(NCc5ccc6c(n5)NC(=O)CO6)(CC3)CO4)c2c1]

¹H NMR (DMSO-d₆) δ 1.02-1.06 (m, 1H), 1.23-1.28 (m, 1H), 1.58-1.71 (m, 8H), 1.84-1.96 (m, 4H), 3.56 (s, 3H), 3.64 (s, 4H), 4.12-4.23 (m, 3H), 4.49 (dd, J=9.0, 5.5 Hz, 1H), 4.59 (s, 2H), 6.63 (d, J=9.8 Hz, 1H), 7.02 (d, J=7.9 Hz, 1H), 7.28 (d, J=7.9 Hz, 1H), 7.41 (d, J=1.8 Hz, 1H), 7.83 (d, J=9.8 Hz, 1H), 8.23 (d, J=1.8 Hz, 1H), 11.14 (s, 1H).

MS (ESI⁺) m/z: 590 (MH⁺).

HRMS (ESI⁺) for C₃₁H₃₆N₅O₇ (MH⁺): calcd, 590.26147. found, 590.26183.

### Example 273

### 4-(2-Hydroxy-2-(4-((3-oxo-3,4-dihydro-2H-pyrido[3,2-b][1,4]oxazin-6-yl)methylamino)-2-oxabicyclo[2.2.2]octan-1-yl)ethyl)-6-(2-hydroxyethoxy)-1,5-naphthyridine-3-carbonitrile (Enantiomer A)

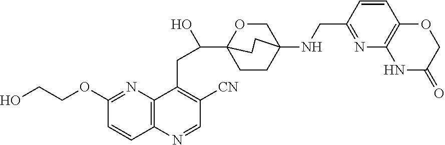 [SMILES: N#Cc1cnc2ccc(OCCO)nc2c1CC(O)C12CCC(NCc3ccc4c(n3)NC(=O)CO4)(CC1)CO2]

The title compound (25.1 mg) was prepared from 4-(2-(4-amino-2-oxabicyclo[2.2.2]octan-1-yl)-2-hydroxyethyl)-6-(2-hydroxyethoxy)-1,5-naphthyridine-3-carbonitrile (25.4 mg) and 3-oxo-3,4-dihydro-2H-pyrido[3,2-b][1,4]oxazine-6-carbaldehyde (12.0 mg) in the same manner as described for Step 3 of EXAMPLE 1.

¹H NMR (DMSO-d₆) δ 1.56-2.05 (m, 8H), 3.01 (dd, J=12.2, 10.4 Hz, 1H), 3.58 (s, 2H), 3.63 (s, 2H), 3.69 (dd, J=12.2, 2.4 Hz, 1H), 3.73-3.82 (m, 3H), 4.49 (t, J=4.9 Hz, 2H), 4.58-4.69 (m, 3H), 4.90 (t, J=5.5 Hz, 1H), 7.01 (d, J=8.0 Hz, 1H), 7.28 (d, J=8.0 Hz, 1H), 7.40 (d, J=8.6 Hz, 1H), 8.32 (d, J=9.2 Hz, 1H), 8.95 (s, 1H), 11.16 (s, 1H).

MS (ESI⁺) m/z: 547 (MH⁺).

HRMS (ESI⁺) for C₂₈H₃₁N₆O₆ (MH⁺): calcd, 547.23051. found, 547.23009.

### Preparation of Intermediates

### Step 1

### Preparation of tert-butyl 1-(2-(3-Cyano-6-oxo-5,6-dihydro-1,5-naphthyridin-4-yl)-1-hydroxyethyl)-2-oxabicyclo[2.2.2]octan-4-ylcarbamate

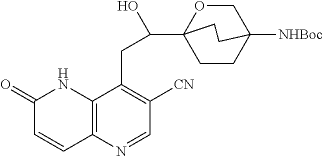 [SMILES: CC(C)(C)OC(=O)NC12CCC(C(O)Cc3c(C#N)cnc4ccc(=O)[nH]c34)(CC1)OC2]

To a solution of tert-butyl 1-(2-(3-cyano-6-oxo-5,6,7,8-tetrahydro-1,5-naphthyridin-4-yl)-1-hydroxyethyl)-2-oxabicyclo[2.2.2]octan-4-ylcarbamate (380 mg) in dioxane (8.6 mL) was added 2,3-dichloro-5,6-dicyano-p-benzoquinone (390 mg), the mixture was stirred at 120° C. for 3 hours and concentrated in vacuo. Flash chromatography (silica, hexane:ethyl acetate=5:2) of the residue gave tert-butyl 1-(2-(3-cyano-6-oxo-5,6-dihydro-1,5-naphthyridin-4-yl)-1-hydroxyethyl)-2-oxabicyclo[2.2.2]octan-4-ylcarbamate (385 mg).

Optical resolution (CHIRALPAK IA, TBME:ethanol=4:1) of the racemate (385 mg) gave Enantiomer A (193 mg) and Enantiomer B (232 mg).

Enantiomer A: ¹H NMR (CDCl₃) δ 1.43 (s, 9H), 1.73-2.04 (m, 6H), 2.11-2.25 (m, 3H), 2.92 (dd, J=14.7, 9.2 Hz, 1H), 3.21 (dd, J=14.7, 1.2 Hz, 1H), 3.66-3.76 (m, 1H), 3.71 (dd, J=9.8, 1.8 Hz, 1H), 3.98-4.12 (m, 2H), 4.34 (brs, 1H), 6.96 (d, J=9.8 Hz, 1H), 7.95 (d, J=9.8 Hz, 1H), 8.66 (s, 1H).

MS (ESI⁺) m/z: 441 (MH⁺).

HRMS (ESI⁺) for C₂₃H₂₉N₄O₅ (MH⁺): calcd, 441.21379. found, 441.21404.

Enantiomer B: ¹H NMR (CDCl₃) δ 1.43 (s, 9H), 1.74-2.04 (m, 6H), 2.12-2.25 (m, 3H), 2.92 (dd, J=14.7, 9.2 Hz, 1H), 3.21 (dd, J=14.7, 1.8 Hz, 1H), 3.67-3.77 (m, 1H), 3.71 (dd, J=9.2, 1.8 Hz, 1H), 3.98-4.11 (m, 2H), 4.34 (brs, 1H), 6.96 (d, J=9.8 Hz, 1H), 7.95 (d, J=9.8 Hz, 1H), 8.66 (s, 1H).

MS (ESI⁺) m/z: 441 (MH⁺).

HRMS (ESI⁺) for C₂₃H₂₉N₄O₅ (MH⁺): calcd, 441.21379. found, 441.21378.

### Step 2

### Preparation of tert-Butyl 1-(2-(3-Cyano-6-(2-(tetrahydro-2H-pyran-2-yloxy)ethoxy)-1,5-naphthyridin-4-yl)-1-hydroxyethyl)-2-oxabicyclo[2.2.2]octan-4-ylcarbamate

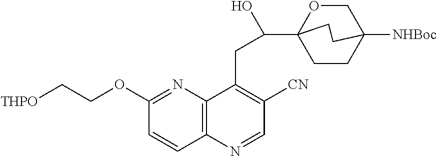

The title compound (64.2 mg) was prepared from tert-butyl 1-(2-(3-cyano-6-oxo-5,6-dihydro-1,5-naphthyridin-4-yl)-1-hydroxyethyl)-2-oxabicyclo[2.2.2]octan-4-ylcarbamate (90 mg) and 2-(2-bromoethoxy)tetrahydro-2H-pyran (50 uL) in the same manner as described for Step 1 of EXAMPLE 32.

Enantiomer A: ¹H NMR (CDCl₃) δ 1.44 (s, 9H), 1.50-1.94 (m, 10H), 2.08-2.26 (m, 4H), 2.73-2.80 (m, 1H), 3.37 (t, J=11.6 Hz, 1H), 3.51-3.58 (m, 1H), 3.61 (dd, J=12.2, 2.4 Hz, 1H), 3.80-3.95 (m, 3H), 4.01 (s, 1H), 4.10-4.18 (m, 1H), 4.01 (s, 2H), 4.10-4.20 (m, 1H), 4.32 (s, 1H), 4.62-4.74 (m, 3H), 7.27 (d, J=8.0 Hz, 1H), 8.23 (d, J=8.6 Hz, 1H), 8.65 (s, 1H).

MS (ESI⁺) m/z: 569 (MH⁺).

HRMS (ESI⁺) for C₃₀H₄₁N₄O₇ (MH⁺): calcd, 569.29752. found, 569.29821.

### Step 3

### Preparation of 4-(2-(4-Amino-2-oxabicyclo[2.2.2]octan-1-yl)-2-hydroxyethyl)-6-(2-hydroxyethoxy)-1,5-naphthyridine-3-carbonitrile

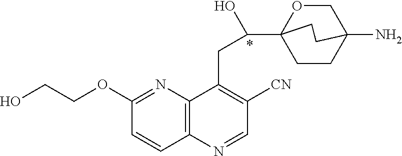 [SMILES: N#Cc1cnc2ccc(OCCO)nc2c1CC(O)C12CCC(N)(CC1)CO2]

The title compound (26.7 mg) was prepared from tert-butyl 1-(2-(3-cyano-6-(2-(tetrahydro-2H-pyran-2-yloxy)ethoxy)-1,5-naphthyridin-4-yl)-1-hydroxyethyl)-2-oxabicyclo[2.2.2]octan-4-ylcarbamate (59.0 mg) in the same manner as described for Step 2 of EXAMPLE 32.

¹H NMR (DMSO-d₆) δ 1.35-1.66 (m, 6H), 1.68-1.86 (m, 3H), 1.88-1.99 (m, 1H), 2.99 (dd, J=12.2, 10.4 Hz, 1H), 3.42-3.50 (m, 2H), 3.68 (dd, J=12.2, 2.4 Hz, 1H), 3.74-3.82 (m, 2H), 4.49 (t, J=4.9 Hz, 1H), 4.57 (d, J=5.5 Hz, 1H), 4.90 (t, J=5.5 Hz, 1H), 7.40 (d, J=9.2 Hz, 1H), 8.32 (d, J=9.2 Hz, 1H), 8.95 (s, 1H).

MS (ESI⁺) m/z: 385 (MH⁺).

HRMS (ESI⁺) for C₂₀H₂₅N₄O₄ (MH⁺): calcd, 385.18758. found, 385.18766.

### Example 274

### 4-(2-Hydroxy-2-(4-((3-oxo-3,4-dihydro-2H-pyrido[3,2-b][1,4]oxazin-6-yl)methylamino)-2-oxabicyclo[2.2.2]octan-1-yl)ethyl)-6-(2-hydroxyethoxy)-1,5-naphthyridine-3-carbonitrile (Enantiomer B)

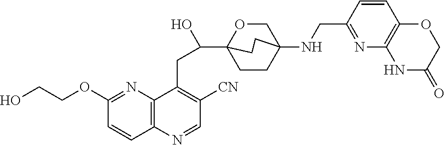 [SMILES: N#Cc1cnc2ccc(OCCO)nc2c1CC(O)C12CCC(NCc3ccc4c(n3)NC(=O)CO4)(CC1)CO2]

The title compound (51.5 mg) was prepared from 4-(2-(4-amino-2-oxabicyclo[2.2.2]octan-1-yl)-2-hydroxyethyl)-6-(2-hydroxyethoxy)-1,5-naphthyridine-3-carbonitrile (48.0 mg) and 3-oxo-3,4-dihydro-2H-pyrido[3,2-b][1,4]oxazine-6-carbaldehyde (23.0 mg) in the same manner as described for Step 3 of EXAMPLE 1.

¹H NMR (DMSO-d₆) δ 1.56-2.05 (m, 8H), 3.01 (dd, J=12.2, 10.4 Hz, 1H), 3.58 (s, 2H), 3.63 (s, 2H), 3.69 (dd, J=12.2, 2.4 Hz, 1H), 3.73-3.82 (m, 3H), 4.49 (t, J=4.9 Hz, 2H), 4.58-4.61 (m, 3H), 4.90 (t, J=5.5 Hz, 1H), 7.01 (d, J=8.0 Hz, 1H), 7.28 (d, J=8.0 Hz, 1H), 7.40 (d, J=8.6 Hz, 1H), 8.32 (d, J=9.2 Hz, 1H), 8.95 (s, 1H), 11.16 (s, 1H).

MS (ESI⁺) m/z: 547 (MH⁺).

HRMS (ESI⁺) for C₂₈H₃₁N₆O₆ (MH⁺): calcd, 547.23051. found, 547.23055.

### Preparation of Intermediates

### Step 1

### tert-Butyl 1-(2-(3-Cyano-6-oxo-5,6-dihydro-1,5-naphthyridin-4-yl)-1-hydroxyethyl)-2-oxabicyclo[2.2.2]octan-4-ylcarbamate

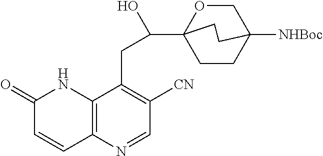 [SMILES: CC(C)(C)OC(=O)NC12CCC(C(O)Cc3c(C#N)cnc4ccc(=O)[nH]c34)(CC1)OC2]

Preparation method is same as step 1 of Example 273.

### Step 2

### Preparation of tert-Butyl 1-(2-(3-Cyano-6-(2-(tetrahydro-2H-pyran-2-yloxy)ethoxy)-1,5-naphthyridin-4-yl)-1-hydroxyethyl)-2-oxabicyclo[2.2.2]octan-4-ylcarbamate

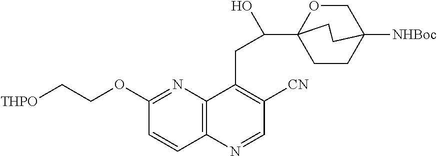

The title compound (64.2 mg) was prepared from tert-butyl 1-(2-(3-cyano-6-oxo-5,6-dihydro-1,5-naphthyridin-4-yl)-1-hydroxyethyl)-2-oxabicyclo[2.2.2]octan-4-ylcarbamate (90 mg) and 2-(2-bromoethoxy)tetrahydro-2H-pyran (50 uL) in the same manner as described for Step 1 of EXAMPLE 32.

¹H NMR (CDCl₃) δ 1.44 (s, 9H), 1.50-1.96 (m, 10H), 2.09-2.28 (m, 4H), 2.72-2.80 (m, 1H), 3.33-3.41 (m, 1H), 3.51-3.58 (m, 1H), 3.61 (dd, J=12.2, 2.4 Hz, 1H), 3.80-3.95 (m, 3H), 4.01 (s, 2H), 4.10-4.18 (m, 1H), 4.31 (s, 1H), 4.58-4.76 (m, 3H), 7.27 (d, J=8.0 Hz, 1H), 8.23 (d, J=9.2 Hz, 1H), 8.65 (s, 1H).

MS (ESI⁺) m/z: 569 (MH⁺).

HRMS (ESI⁺) for C₃₀H₄₁N₄O₇ (MH⁺): calcd, 569.29752. found, 569.29697.

### Step 3

### Preparation of 4-(2-(4-Amino-2-oxabicyclo[2.2.2]octan-1-yl)-2-hydroxyethyl)-6-(2-hydroxyethoxy)-1,5-naphthyridine-3-carbonitrile

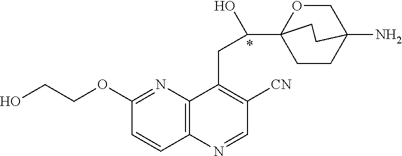 [SMILES: N#Cc1cnc2ccc(OCCO)nc2c1CC(O)C12CCC(N)(CC1)CO2]

The title compound (51.2 mg) was prepared from tert-butyl 1-(2-(3-cyano-6-(2-(tetrahydro-2H-pyran-2-yloxy)ethoxy)-1,5-naphthyridin-4-yl)-1-hydroxyethyl)-2-oxabicyclo[2.2.2]octan-4-ylcarbamate (86.0 mg) in the same manner as described for Step 2 of EXAMPLE 32.

¹H NMR (DMSO-d₆) δ 1.40-1.66 (m, 6H), 1.68-1.86 (m, 3H), 1.87-2.00 (m, 1H), 2.99 (dd, J=12.2, 10.4 Hz, 1H), 3.41-3.50 (m, 2H), 3.68 (dd, J=12.2, 2.4 Hz, 1H), 3.72-3.82 (m, 2H), 4.49 (t, J=4.9 Hz, 1H), 4.57 (d, J=5.5 Hz, 1H), 4.90 (t, J=5.5 Hz, 1H), 7.39 (d, J=9.2 Hz, 1H), 8.32 (d, J=9.2 Hz, 1H), 8.95 (s, 1H).

MS (ESI⁺) m/z: 385 (MH⁺).

HRMS (ESI⁺) for C₂₀H₂₅N₄O₄ (MH⁺): calcd, 385.18758. found, 385.18686.

### Example 275

### 4-(2-Hydroxy-2-(4-((3-oxo-3,4-dihydro-2H-pyrido[3,2-b][1,4]oxazin-6-yl)methylamino)-2-oxabicyclo[2.2.2]octan-1-yl)ethyl)-6-(3-hydroxypropoxy)-1,5-naphthyridine-3-carbonitrile (Enantiomer A)

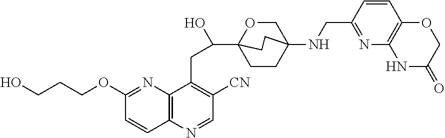 [SMILES: N#Cc1cnc2ccc(OCCCO)nc2c1CC(O)C12CCC(NCc3ccc4c(n3)NC(=O)CO4)(CC1)CO2]

The title compound (50.6 mg) was prepared from 4-(2-(4-amino-2-oxabicyclo[2.2.2]octan-1-yl)-2-hydroxyethyl)-6-(3-hydroxypropoxy)-1,5-naphthyridine-3-carbonitrile (64.0 mg) and 3-oxo-3,4-dihydro-2H-pyrido[3,2-b][1,4]oxazine-6-carbaldehyde (29.0 mg) in the same manner as described for Step 3 of EXAMPLE 1.

¹H NMR (DMSO-d₆) δ 1.56-2.04 (m, 10H), 1.90-2.04 (m, 4H), 3.00 (dd, J=12.2, 10.4 Hz, 1H), 3.56-3.80 (m, 8H), 4.51-4.62 (m, 6H), 7.01 (d, J=7.9 Hz, 1H), 7.28 (d, J=7.9 Hz, 1H), 7.38 (d, J=8.6 Hz, 1H), 8.31 (d, J=9.2 Hz, 1H), 8.95 (s, 1H), 11.15 (s, 1H).

MS (ESI⁺) m/z: 561 (MH⁺).

HRMS (ESI⁺) for C₂₉H₃₃N₆O₆ (MH⁺): calcd, 561.24616. found, 561.24612.

### Preparation of Intermediates

### Step 1

### Preparation of tert-Butyl 1-(2-(3-Cyano-6-(3-(tetrahydro-2H-pyran-2-yloxy)propoxy)-1,5-naphthyridin-4-yl)-1-hydroxyethyl)-2-oxabicyclo[2.2.2]octan-4-ylcarbamate

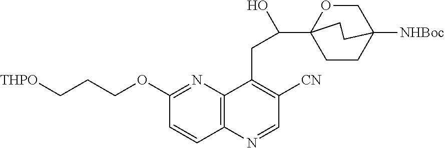

The title compound (101 mg) was prepared from tert-butyl 1-(2-(3-cyano-6-oxo-5,6-dihydro-1,5-naphthyridin-4-yl)-1-hydroxyethyl)-2-oxabicyclo[2.2.2]octan-4-ylcarbamate (93.0 mg) and 2-(3-bromopropoxy)tetrahydro-2H-pyran (44 uL) in the same manner as described for Step 1 of EXAMPLE 32.

¹H NMR (CDCl₃) δ 1.44 (s, 9H), 1.51-1.61 (m, 4H), 1.68-1.96 (m, 6H), 2.10-2.24 (m, 6H), 2.80-2.87 (m, 1H), 3.32-3.41 (m, 1H), 3.47-3.54 (m, 1H), 3.56-3.65 (m, 2H), 3.81-3.90 (m, 2H), 3.92-4.05 (m, 3H), 4.31 (s, 1H), 4.53-4.64 (m, 3H), 7.21 (d, J=8.6 Hz, 1H), 8.22 (d, J=9.2 Hz, 1H), 8.85 (s, 1H).

MS (ESI⁺) m/z: 583 (MH⁺).

HRMS (ESI⁺) for C₃₁H₄₃N₄O₇ (MH⁺): calcd, 583.31317. found, 583.31278.

### Step 2

### Preparation of 4-(2-(4-Amino-2-oxabicyclo[2.2.2]octan-1-yl)-2-hydroxyethyl)-6-(3-hydroxypropoxy)-1,5-naphthyridine-3-carbonitrile

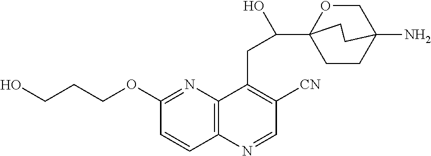 [SMILES: N#Cc1cnc2ccc(OCCCO)nc2c1CC(O)C12CCC(N)(CC1)CO2]

The title compound (66.6 mg) was prepared from tert-butyl 1-(2-(3-cyano-6-(3-(tetrahydro-2H-pyran-2-yloxy)propoxy)-1,5-naphthyridin-4-yl)-1-hydroxyethyl)-2-oxabicyclo[2.2.2]octan-4-ylcarbamate (90.0 mg) in the same manner as described for Step 2 of EXAMPLE 32.

¹H NMR (DMSO-d₆) δ 1.50-1.66 (m, 4H), 1.70-1.84 (m, 3H), 1.90-2.00 (m, 3H), 2.99 (dd, J=12.2, 10.4 Hz, 1H), 3.45-3.51 (m, 2H), 3.57 (dd, J=11.0, 6.1 Hz, 2H), 3.70 (dd, J=11.6, 2.4 Hz, 1H), 3.73-3.76 (m, 1H), 4.51-4.59 (m, 4H), 7.37 (d, J=9.2 Hz, 1H), 8.31 (d, J=9.2 Hz, 1H), 8.94 (s, 1H).

MS (ESI⁺) m/z: 399 (MH⁺).

HRMS (ESI⁺) for C₂₁H₂₇N₄O₄ (MH⁺): calcd, 399.20323. found, 399.20288.

### Example 276

### 4-(2-Hydroxy-2-(4-((3-oxo-3,4-dihydro-2H-pyrido[3,2-b][1,4]oxazin-6-yl)methylamino)-2-oxabicyclo[2.2.2]octan-1-yl)ethyl)-6-(3-hydroxypropoxy)-1,5-naphthyridine-3-carbonitrile (Enantiomer B)

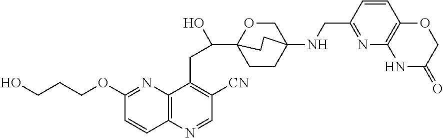 [SMILES: N#Cc1cnc2ccc(OCCCO)nc2c1CC(O)C12CCC(NCc3ccc4c(n3)NC(=O)CO4)(CC1)CO2]

The title compound (58.1 mg) was prepared from 4-(2-(4-amino-2-oxabicyclo[2.2.2]octan-1-yl)-2-hydroxyethyl)-6-(3-hydroxypropoxy)-1,5-naphthyridine-3-carbonitrile (66.5 mg) and 3-oxo-3,4-dihydro-2H-pyrido[3,2-b][1,4]oxazine-6-carbaldehyde (30.0 mg) in the same manner as described for Step 3 of EXAMPLE 1.

¹H NMR (DMSO-d₆) δ 1.56-2.04 (m, 10H), 3.00 (dd, J=12.2, 10.4 Hz, 1H), 3.56-3.80 (m, 8H), 4.51-4.62 (m, 6H), 7.01 (d, J=7.9 Hz, 1H), 7.28 (d, J=8.6 Hz, 1H), 7.38 (d, J=9.2 Hz, 1H), 8.31 (d, J=9.2 Hz, 1H), 8.95 (s, 1H), 11.15 (s, 1H).

MS (ESI⁺) m/z: 561 (MH⁺).

HRMS (ESI⁺) for C₂₉H₃₃N₆O₆ (MH⁺): calcd, 561.24616. found, 561.24607.

### Preparation of Intermediates

### Step 1

### Preparation of tert-Butyl 1-(2-(3-Cyano-6-(3-(tetrahydro-2H-pyran-2-yloxy)propoxy)-1,5-naphthyridin-4-yl)-1-hydroxyethyl)-2-oxabicyclo[2.2.2]octan-4-ylcarbamate

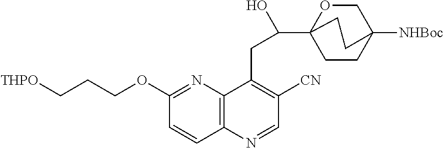

The title compound (107 mg) was prepared from tert-butyl 1-(2-(3-cyano-6-oxo-5,6-dihydro-1,5-naphthyridin-4-yl)-1-hydroxyethyl)-2-oxabicyclo[2.2.2]octan-4-ylcarbamate (90.0 mg) and 2-(3-bromopropoxy)tetrahydro-2H-pyran (46 uL) in the same manner as described for Step 1 of EXAMPLE 32.

¹H NMR (CDCl₃) δ 1.44 (s, 9H), 1.66-1.96 (m, 6H), 2.10-2.24 (m, 6H), 2.80-2.87 (m, 1H), 3.36 (dd, J=11.6, 10.4 Hz, 1H), 3.47-3.55 (m, 1H), 3.56-3.65 (m, 2H), 3.80-3.90 (m, 2H), 3.92-4.04 (m, 3H), 4.31 (s, 1H), 4.53-4.64 (m, 3H), 7.21 (d, J=9.2 Hz, 1H), 8.22 (d, J=9.2 Hz, 1H), 8.85 (s, 1H).

MS (ESI⁺) m/z: 583 (MH⁺).

HRMS (ESI⁺) for C₃₁H₄₃N₄O₇ (MH⁺): calcd, 583.31317. found, 583.31313.

### Step 2

### Preparation of 4-(2-(4-Amino-2-oxabicyclo[2.2.2]octan-1-yl)-2-hydroxyethyl)-6-(3-hydroxypropoxy)-1,5-naphthyridine-3-carbonitrile

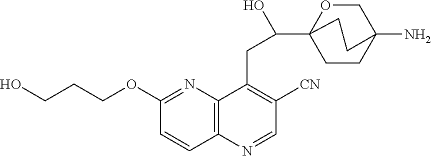 [SMILES: N#Cc1cnc2ccc(OCCCO)nc2c1CC(O)C12CCC(N)(CC1)CO2]

The title compound (70.6 mg) was prepared from tert-butyl 1-(2-(3-cyano-6-(3-(tetrahydro-2H-pyran-2-yloxy)propoxy)-1,5-naphthyridin-4-yl)-1-hydroxyethyl)-2-oxabicyclo[2.2.2]octan-4-ylcarbamate (101 mg) in the same manner as described for Step 2 of EXAMPLE 32.

¹H NMR (DMSO-d₆) δ 1.48-1.66 (m, 4H), 1.69-1.86 (m, 3H), 1.94 (quintet, J=6.1 Hz, 3H), 2.99 (dd, J=12.2, 10.4 Hz, 1H), 3.44-3.51 (m, 2H), 3.57 (dd, J=11.6, 6.1 Hz, 2H), 3.70 (dd, J=11.6, 2.4 Hz, 1H), 3.73-3.80 (m, 1H), 4.51-4.60 (m, 4H), 7.38 (d, J=9.2 Hz, 1H), 8.31 (d, J=8.6 Hz, 1H), 8.94 (s, 1H).

MS (ESI⁺) m/z: 399 (MH⁺).

HRMS (ESI⁺) for C₂₁H₂₇N₄O₄ (MH⁺): calcd, 399.20323. found, 399.20343.

### Example 277

The following compound was prepared consistent with the methods described herein.

### Methyl 2-((6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridin-3-yloxy)methyl)cyclopropanecarboxylate

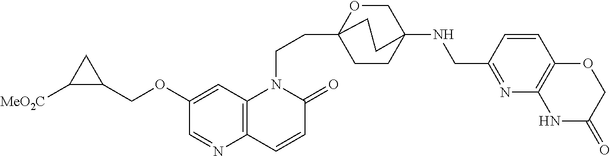 [SMILES: COC(=O)C1CC1COc1cnc2ccc(=O)n(CCC34CCC(NCc5ccc6c(n5)NC(=O)CO6)(CC3)CO4)c2c1]

H NMR (DMSO-d₆) δ 1.02-1.08 (m, 1H), 1.14-1.19 (m, 1H), 1.58-1.71 (m, 8H), 1.80-1.90 (m, 4H), 3.60 (s, 3H), 3.63 (s, 4H), 4.03 (dd, J=10.7, 7.6 Hz, 1H), 4.20-4.24 (m, 3H), 4.59 (s, 2H), 6.64 (d, J=9.8 Hz, 1H), 7.02 (d, J=8.6 Hz, 1H), 7.29 (d, J=7.9 Hz, 1H), 7.41 (d, J=2.4 Hz, 1H), 7.84 (d, J=9.8 Hz, 1H), 8.28 (d, J=2.4 Hz, 1H), 11.16 (s, 1H).

MS (ESI⁺) m/z: 590 (MH⁺).

HRMS (ESI⁺) for C₃₁H₃₆N₅O₇ (MH⁺): calcd, 590.26147. found, 590.26163.

### Example 278

The following compound was prepared consistent with the methods described herein.

### Ethyl 2,2-Difluoro-4-(7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanoate

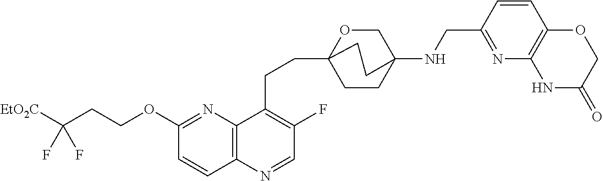 [SMILES: CCOC(=O)C(F)(F)CCOc1ccc2ncc(F)c(CCC34CCC(NCc5ccc6c(n5)NC(=O)CO6)(CC3)CO4)c2n1]

¹H NMR (CDCl₃) δ 1.32 (t, J=7.0 Hz, 3H), 1.70-1.87 (m, 8H), 1.98-2.08 (m, 2H), 2.71 (tt, J=15.9, 6.1 Hz, 2H), 3.15-3.23 (m, 2H), 3.76 (s, 2H), 3.77 (s, 2H), 4.33 (q, J=7.0 Hz, 2H), 4.63 (s, 2H), 4.72 (t, J=6.1 Hz, 2H), 6.94 (d, J=7.9 Hz, 1H), 7.00 (d, J=9.2 Hz, 1H), 7.20 (d, J=7.9 Hz, 1H), 8.19 (d, J=8.6 Hz, 1H), 8.62 (s, 1H).

MS (ESI⁺) m/z: 630 (MH⁺).

HRMS (ESI⁺) for C₃₁H₃₅F₃N₅O₆ (MH⁺): calcd, 630.25394. found, 630.25401.

### Example 279

The following compound was prepared consistent with the methods described herein.

### 2,2-Difluoro-4-(7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanamide

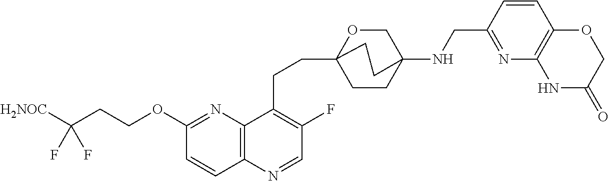 [SMILES: NC(=O)C(F)(F)CCOc1ccc2ncc(F)c(CCC34CCC(NCc5ccc6c(n5)NC(=O)CO6)(CC3)CO4)c2n1]

¹H NMR (DMSO-d₆) δ 1.57-1.74 (m, 10H), 2.57-2.72 (m, 2H), 3.06-3.13 (m, 2H), 3.58 (s, 2H), 3.62 (s, 2H), 4.59 (s, 2H), 4.63 (t, J=6.1 Hz), 7.01 (d, J=7.9 Hz, 1H), 7.17 (d, J=9.2 Hz, 1H), 7.28 (d, J=7.9 Hz, 1H), 7.93 (s, 1H), 8.15 (s, 1H), 8.28 (d, J=9.2 Hz, 1H), 8.76 (s, 1H), 11.16 (s, 1H).

MS (ESI⁺) m/z: 601 (MH⁺).

HRMS (ESI⁺) for C₂₉H₃₂F₃N₆O₅ (MH⁺): calcd, 601.23863. found 601.23847.

### Example 280

The following compound was prepared consistent with the methods described herein.

### Methyl 2-((6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridin-3-yloxy)methyl)cyclopropanecarboxylate

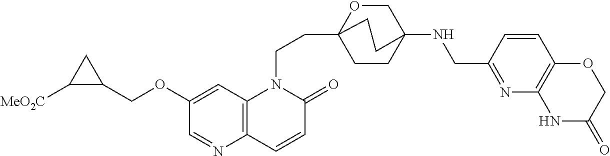 [SMILES: COC(=O)C1CC1COc1cnc2ccc(=O)n(CCC34CCC(NCc5ccc6c(n5)NC(=O)CO6)(CC3)CO4)c2c1]

¹H NMR (DMSO-d₆) δ 1.02-1.07 (m, 1H), 1.13-1.18 (m, 1H), 1.56-1.68 (m, 8H), 1.79-1.93 (m, 4H), 3.60 (s, 3H), 3.62 (s, 4H), 4.02 (dd, J=10.4, 7.3 Hz, 1H), 4.18-4.22 (m, 3H), 4.58 (s, 2H), 6.62 (d, J=9.8 Hz, 1H), 7.01 (d, J=7.9 Hz, 1H), 7.27 (d, J=7.9 Hz, 1H), 7.40 (d, J=1.8 Hz, 1H), 7.83 (d, J=9.8 Hz, 1H), 8.27 (d, J=2.4 Hz, 1H), 11.14 (s, 1H).

MS (ESI⁺) m/z: 590 (MH⁺).

HRMS (ESI⁺) for C₃₁H₃₆N₅O₇ (MH⁺): calcd, 590.26147. found, 590.26120.

### Example 281

The following compound was prepared consistent with the methods described herein.

### 2,2-Difluoro-4-(7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanoic Acid

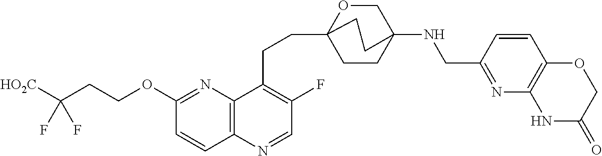 [SMILES: O=C1COc2ccc(CNC34CCC(CCc5c(F)cnc6ccc(OCCC(F)(F)C(=O)O)nc56)(CC3)OC4)nc2N1]

¹H NMR (DMSO-d₆) δ 1.62-1.80 (m, 4H), 1.84-2.07 (m, 6H), 2.48-2.58 (m, 2H), 3.15 (t, J=7.3 Hz, 2H), 3.77 (s, 2H), 4.03 (s, 2H), 4.58 (t, J=6.7 Hz, 2H), 4.65 (s, 2H), 7.16 (d, J=7.9 Hz, 1H), 7.20 (d, J=9.1 Hz, 1H), 7.39 (d, J=7.9 Hz, 1H), 8.25 (d, J=9.1 Hz, 1H), 8.74 (s, 1H), 11.41 (s, 1H).

MS (ESI⁺) m/z: 602 (MH⁺).

HRMS (ESI⁺) for C₂₉H₃₁F₃N₅O₆ (MH⁺): calcd, 602.22264. found 602.22228.

### Example 282

### 1-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarbonitrile

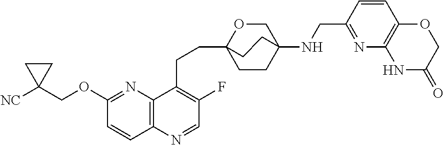 [SMILES: N#CC1(COc2ccc3ncc(F)c(CCC45CCC(NCc6ccc7c(n6)NC(=O)CO7)(CC4)CO5)c3n2)CC1]

The title compound (109 mg) was prepared from 1-((8-(2-(4-amino-2-oxabicyclo[2.2.2]octan-1-yl)ethyl)-7-fluoro-1,5-naphthyridin-2-yloxy)methyl)cyclopropanecarbonitrile (94 mg) and 3-oxo-3,4-dihydro-2H-pyrido[3,2-b][1,4]oxazine-6-carbaldehyde (42.2 mg) in the same manner as described for Step 3 of EXAMPLE 1.

¹H NMR (DMSO-d₆) δ 1.23 (dd, J=7.3, 4.9 Hz, 2H), 1.42 (dd, J=7.3, 4.9 Hz, 2H), 1.58-1.76 (m, 8H), 1.80-1.94 (m, 2H), 3.04-3.14 (m, 2H), 3.59 (s, 2H), 3.63 (s, 2H), 4.56 (s, 2H), 4.59 (s, 2H), 7.01 (d, J=8.6 Hz, 1H), 7.28 (d, J=8.0 Hz, 1H), 7.32 (d, J=8.6 Hz, 1H), 8.31 (d, J=9.2 Hz, 1H), 8.77 (s, 1H), 11.16 (s, 1H).

MS (ESI⁺) m/z: 559 (MH⁺).

HRMS (ESI⁺) for C₃₀H₃₂FN₆O₄ (MH⁺): calcd, 559.24691. found, 559.24634.

### Preparation of Intermediates

### Step 1

### Preparation of tert-Butyl 1-(2-(6-((1-Cyanocyclopropyl)methoxy)-3-fluoro-1,5-naphthyridin-4-yl)ethyl)-2-oxabicyclo[2.2.2]octan-4-ylcarbamate

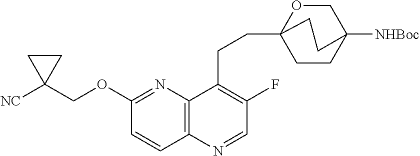 [SMILES: CC(C)(C)OC(=O)NC12CCC(CCc3c(F)cnc4ccc(OCC5(C#N)CC5)nc34)(CC1)OC2]

The title compound (51.5 mg) was prepared from tert-butyl 1-(2-(3-fluoro-6-hydroxy-1,5-naphthyridin-4-yl)ethyl)-2-oxabicyclo[2.2.2]octan-4-ylcarbamate (100 mg) and 1-(bromomethyl)cyclopropanecarbonitrile (58.0 mg) in the same manner as described for Step 1 of EXAMPLE 32.

¹H NMR (CDCl₃) δ 1.21 (dd, J=7.3, 5.5 Hz, 2H), 1.40-1.48 (m, 11H), 1.67-1.79 (m, 4H), 1.82-1.94 (m, 2H), 1.96-2.06 (m, 2H), 2.07-2.18 (m, 2H), 3.10-3.20 (m, 2H), 3.97 (s, 2H), 4.30 (brs, 1H), 4.54 (s, 2H), 7.14 (d, J=9.2 Hz, 1H), 8.22 (d, J=9.2 Hz, 1H), 8.62 (s, 1H).

MS (ESI⁺) m/z: 497 (MH⁺).

HRMS (ESI⁺) for C₂₇H₃₄FN₄O₄ (MH⁺): calcd, 497.25641. found, 497.25550.

### Step 2

### Preparation of 1-((8-(2-(4-Amino-2-oxabicyclo[2.2.2]octan-1-yl)ethyl)-7-fluoro-1,5-naphthyridin-2-yloxy)methyl)cyclopropanecarbonitrile

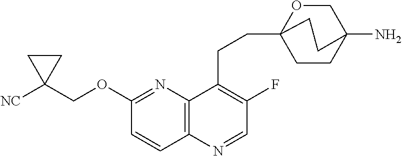 [SMILES: N#CC1(COc2ccc3ncc(F)c(CCC45CCC(N)(CC4)CO5)c3n2)CC1]

The title compound (96.9 mg) was prepared from tert-butyl 1-(2-(6-((1-cyanocyclopropyl)methoxy)-3-fluoro-1,5-naphthyridin-4-yl)ethyl)-2-oxabicyclo[2.2.2]octan-4-ylcarbamate (118 mg) in the same manner as described for Step 2 of EXAMPLE 32.

¹H NMR (CDCl₃) δ: 1.21 (dd, J=7.3, 4.9 Hz, 2H), 1.43 (dd, J=7.3, 4.9 Hz, 2H), 1.63-1.81 (m, 8H), 1.92-2.06 (m, 2H), 3.10-3.21 (m, 2H), 3.65 (s, 2H), 4.55 (s, 2H), 7.14 (d, J=9.2 Hz, 1H), 8.22 (d, J=9.2 Hz, 1H), 8.62 (s, 1H).

MS (ESI⁺) m/z: 397 (MH⁺).

HRMS (ESI⁺) for C₂₂H₂₆FN₄O₂ (MH⁺): calcd, 397.20398. found, 397.20482.

### Example 283

The following compound was prepared consistent with the methods described herein.

2-((6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridin-3-yloxy)methyl)cyclopropanecarboxylic Acid Hydrochloride

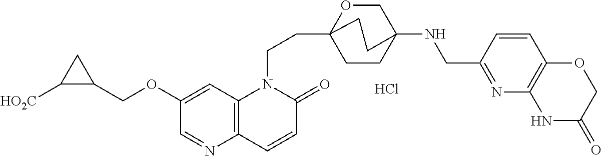 [SMILES: O=C1COc2ccc(CNC34CCC(CCn5c(=O)ccc6ncc(OCC7CC7C(=O)O)cc65)(CC3)OC4)nc2N1 ; Cl]

¹H NMR (DMSO-d₆) δ 0.99-1.06 (m, 1H), 1.12-1.16 (m, 1H), 1.64-1.71 (m, 3H), 1.76-1.90 (m, 3H), 1.90-2.10 (m, 6H), 3.97 (s, 2H), 4.04 (dd, J=10.7, 7.6 Hz, 1H), 4.10 (s, 2H), 4.21-4.26 (m, 3H), 4.68 (s, 2H), 6.65 (d, J=9.2 Hz, 1H), 7.24 (d, J=7.9 Hz, 1H), 7.34 (d, J=2.4 Hz, 1H), 7.45 (d, J=7.9 Hz, 1H), 7.85 (d, J=9.8 Hz, 1H), 8.30 (d, J=2.4 Hz, 1H), 9.37 (s, 2H), 11.33 (s, 1H), 12.29 (brs, 1H).

MS (ESI⁺) m/z: 576 (MH⁺) (as free base).

HRMS (ESI⁺) for C₃₀H₃₄N₅O₇ (MH⁺) (as free base): calcd, 576.24582. found, 576.24540.

### Example 284

The following compound was prepared consistent with the methods described herein.

### 6-((1-(2-(6-(Difluoromethoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one

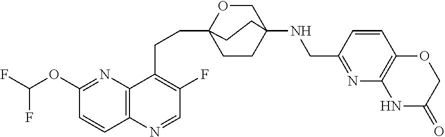 [SMILES: O=C1COc2ccc(CNC34CCC(CCc5c(F)cnc6ccc(OC(F)F)nc56)(CC3)OC4)nc2N1]

¹H NMR (DMSO-d₆) δ 1.60-1.74 (m, 8H), 1.84-1.89 (m, 2H), 3.07-3.11 (m, 2H), 3.58 (s, 2H), 3.62 (s, 2H), 4.59 (s, 2H), 7.01 (d, J=7.9 Hz, 1H), 7.27 (d, J=7.9 Hz, 1H), 7.46 (d, J=8.6 Hz, 1H), 7.90 (t, J=72.1 Hz, 1H), 8.51 (d, J=9.2 Hz, 1H), 8.91 (s, 1H), 11.16 (s, 1H).

MS (ESI⁺) m/z: 530 (MH⁺).

HRMS (ESI⁺) for C₂₆H₂₇F₃N₅O₄ (MH⁺): calcd, 530.20151. found, 530.20133.

### Example 285

The following compound was prepared consistent with the methods described herein.

### 5,8-Difluoro-3-((1-(2-(3-fluoro-6-methoxy-1,5-naphthyridin-4-yl)ethyl)-2-oxabicyclo[2.2.2]octan-4-ylamino)methyl)-1-methylquinolin-2(1H)-one

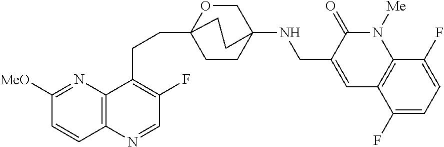 [SMILES: COc1ccc2ncc(F)c(CCC34CCC(NCc5cc6c(F)ccc(F)c6n(C)c5=O)(CC3)CO4)c2n1]

¹H NMR (DMSO-d₆) δ 1.60-1.80 (m, 8H), 1.83-1.95 (m, 2H), 2.00-2.20 (m, 1H), 3.09-3.16 (m, 2H), 3.58 (brs, 2H), 3.63 (brs, 2H), 3.79 (d, J=8.6 Hz, 3H), 4.03 (s, 3H), 7.13 (td, J=8.6, 3.1 Hz, 1H), 7.22 (d, J=9.2 Hz, 1H), 7.45 (td, J=8.6, 3.7 Hz, 1H), 7.99 (brs, 1H), 8.25 (d, J=8.6 Hz, 1H), 8.74 (s, 1H).

MS (ESI⁺) m/z: 539 (MH⁺).

HRMS (ESI⁺) for C₂₉H₃₀F₃N₄O₃ (MH⁺): calcd, 539.22700. found 539.00683.

### Example 286

The following compound was prepared consistent with the methods described herein.

### 6-((1-(2-(3-Fluoro-1,5-naphthyridin-4-yl)ethyl)-2-oxabicyclo[2.2.2]octan-4-ylamino)methyl)-2H-pyrido[3,2-b][1,4]oxazin-3(4H)-one

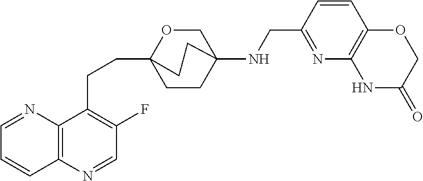 [SMILES: O=C1COc2ccc(CNC34CCC(CCc5c(F)cnc6cccnc56)(CC3)OC4)nc2N1]

¹H NMR (DMSO-d₆) δ 1.57-1.75 (m, 8H), 1.78-1.93 (m, 2H), 3.19-3.27 (m, 2H), 3.57 (s, 2H), 3.63 (d, J=4.3 Hz, 2H), 4.59 (s, 2H), 7.01 (d, J=7.9 Hz, 1H), 7.28 (d, J=7.9 Hz, 1H), 7.77 (dd, J=8.6, 4.3 Hz, 1H), 8.44 (dd, J=8.6, 1.8 Hz, 1H), 8.98 (s, 1H), 9.05 (dd, J=4.3, 1.8 Hz, 1H), 11.15 (s, 1H).

MS (ESI⁺) m/z: 464 (MH⁺).

HRMS (ESI⁺) for C₂₅H₂₇FN₅O₃ (MH⁺): calcd, 464.20979. found, 464.21063.

### Example 287

The following compound was prepared consistent with the methods described herein.

### 2-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)-N-methylacetamide

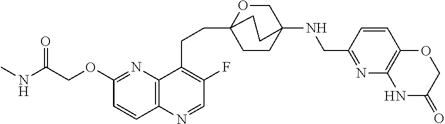 [SMILES: CNC(=O)COc1ccc2ncc(F)c(CCC34CCC(NCc5ccc6c(n5)NC(=O)CO6)(CC3)CO4)c2n1]

¹H NMR (DMSO-d₆) δ 1.51-1.93 (m, 10H), 2.62 (d, J=4.2 Hz), 2.67-3.08 (m, 2H), 3.57 (s, 2H), 3.63 (d, J=5.4 Hz, 2H), 3.64 (s, 2H), 3.84 (dd, J=10.4, 1.2 Hz, 1H), 4.00 (dd, J=8.6, 5.5 Hz, 1H), 4.24 (dd, J=10.4, 4.9 Hz, 2H), 4.59 (s, 2H), 4.89 (s, 2H), 7.01 (d, J=7.9 Hz, 1H), 7.28 (d, J=8.5 Hz, 1H), 7.30 (d, J=9.1 Hz, 1H), 8.00 (q, J=4.2 Hz, 1H), 8.31 (d, J=9.1 Hz, 1H), 8.76 (s, 1H), 11.15 (s, 1H).

MS (ESI⁺) m/z: 551 (MH⁺).

HRMS (ESI⁺) for C₂₈H₃₂FN₆O₅ (MH⁺): calcd, 551.24182. found, 551.24149.

### Example 288

The following compound was prepared consistent with the methods described herein.

### N-((5,8-Difluoro-2-methoxyquinolin-3-yl)methyl)-1-(2-(3-fluoro-6-methoxy-1,5-naphthyridin-4-yl)ethyl)-2-oxabicyclo[2.2.2]octan-4-amine

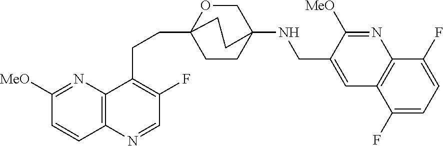 [SMILES: COc1ccc2ncc(F)c(CCC34CCC(NCc5cc6c(F)ccc(F)c6nc5OC)(CC3)CO4)c2n1]

¹H NMR (DMSO-d₆) δ 1.60-1.80 (m, 8H), 1.82-1.94 (m, 2H), 2.16-2.26 (m, 1H), 3.08-3.16 (m, 2H), 3.57 (brs, 2H), 3.63 (brs, 2H), 4.03 (s, 3H), 4.09 (s, 3H), 7.18-7.28 (m, 1H), 7.22 (d, J=9.2 Hz, 1H), 7.46 (tdd, J=8.6, 3.7, 1.8 Hz, 1H), 8.26 (d, J=9.2 Hz, 1H), 8.36 (brs, 1H), 8.75 (s, 1H).

MS (ESI⁺) m/z: 539 (MH⁺)

HRMS (ESI⁺) for C₂₉H₃₀F₃N₄O₃ (MH⁺): calcd, 539.22700. found 539.22626.

### Example 289

The following compound was prepared consistent with the methods described herein.

### N-(2-(2,5-Difluorophenoxy)ethyl)-1-(2-(3-fluoro-6-methoxy-1,5-naphthyridin-4-yl)ethyl)-2-oxabicyclo[2.2.2]octan-4-amine

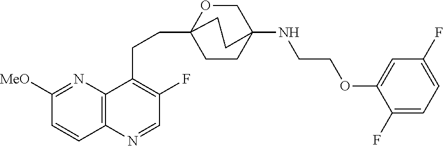 [SMILES: COc1ccc2ncc(F)c(CCC34CCC(NCCOc5cc(F)ccc5F)(CC3)CO4)c2n1]

¹H NMR (DMSO-d₆) δ 1.56-1.78 (m, 9H), 1.80-1.92 (m, 2H), 2.80-2.88 (m, 2H), 3.08-3.14 (m, 2H), 3.56 (brs, 2H), 3.98-4.04 (m, 2H), 4.03 (s, 3H), 6.70-6.78 (m, 1H), 7.10 (tdd, J=10.4, 6.7, 3.1 Hz, 1H), 7.18-7.28 (m, 1H), 7.22 (d, J=9.2 Hz, 1H), 8.26 (d, J=9.2 Hz, 1H), 8.74 (s, 1H).

MS (ESI⁺) m/z: 488 (MH⁺).

HRMS (ESI⁺) for C₂₆H₂₉F₃N₃O₃ (MH⁺): calcd, 488.21610. found 488.21640.

### Example 290

The following compound was prepared consistent with the methods described herein.

### 4-(7-Fluoro-8-((2-(4-((3-oxo-3,4-dihydro-2H-pyrido[3,2-b][1,4]oxazin-6-yl)methyl)amino)-2-oxabicyclo[2.2.2]octan-1-yl)ethyl)-1,5-naphthyridin-2-yl)thiomorpholine-1,1-dioxide

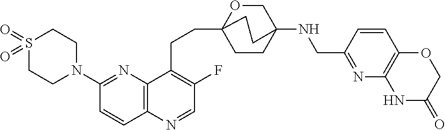 [SMILES: O=C1COc2ccc(CNC34CCC(CCc5c(F)cnc6ccc(N7CCS(=O)(=O)CC7)nc56)(CC3)OC4)nc2N1]

¹H NMR (DMSO-d₆) δ 1.53-1.93 (m, 10H), 2.98-3.09 (m, 2H), 3.17-3.25 (m, 2H), 3.60 (s, 2H), 3.63 (d, J=7.3 Hz, 2H), 4.21-4.31 (m, 4H), 4.59 (s, 2H), 7.01 (d, J=7.9 Hz, 1H), 7.28 (d, J=8.6 Hz, 1H), 7.57 (d, J=9.2 Hz, 1H), 8.13 (d, J=9.2 Hz, 1H), 8.57 (s, 1H), 11.15 (s, 1H).

MS (ESI⁺) m/z: 597 (MH⁺).

HRMS (ESI⁺) for C₂₉H₃₄FN₆O₅S (MH⁺): calcd, 597.22954. found, 597.22904.

### Example 291

The following compound was prepared consistent with the methods described herein.

### 2-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)-N,N-dimethylacetamide

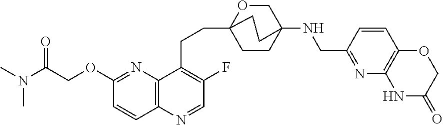 [SMILES: CN(C)C(=O)COc1ccc2ncc(F)c(CCC34CCC(NCc5ccc6c(n5)NC(=O)CO6)(CC3)CO4)c2n1]

¹H NMR (DMSO-d₆) δ 1.47-1.96 (m, 10H), 2.84 (s, 3H), 2.97-3.07 (m, 2H), 3.04 (s, 3H), 3.58 (s, 2H), 3.63 (d, J=6.7 Hz, 2H), 4.59 (s, 2H), 5.24 (s, 2H), 7.01 (d, J=8.6 Hz, 1H), 7.28 (d, J=7.9 Hz, 1H), 7.30 (d, J=9.2 Hz, 1H), 8.28 (d, J=9.2 Hz, 1H), 8.74 (s, 1H), 11.15 (s, 1H).

MS (ESI⁺) m/z: 565 (MH⁺).

HRMS (ESI⁺) for C₂₉H₃₄FN₆O₅ (MH⁺): calcd, 565.25747. found, 565.25780.

### Example 292

The following compound was prepared consistent with the methods described herein.

### 6-((1-(2-(3-Fluoro-6-(2-oxooxazolidin-3-yl)-1,5-naphthyridin-4-yl)ethyl)-2-oxabicyclo[2.2.2]octan-4-ylamino)methyl)-2H-pyrido[3,2-b][1,4]oxazin-3(4H)-one

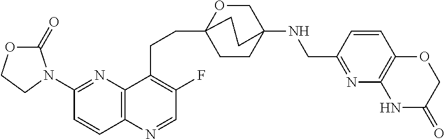 [SMILES: O=C1COc2ccc(CNC34CCC(CCc5c(F)cnc6ccc(N7CCOC7=O)nc56)(CC3)OC4)nc2N1]

¹H NMR (DMSO-d₆) δ 1.56-1.77 (m, 8H), 1.78-1.94 (m, 2H), 3.07-3.13 (m, 2H), 3.58 (s, 2H), 3.63 (d, J=4.3 Hz, 2H), 4.32 (t, J=7.9 Hz, 2H), 4.55 (t, J=7.9 Hz, 2H), 4.59 (s, 2H), 7.01 (d, J=8.6 Hz, 1H), 7.28 (d, J=7.9 Hz, 1H), 8.43 (d, J=9.2 Hz, 1H), 8.52 (d, J=9.2 Hz, 1H), 8.83 (s, 1H), 11.15 (s, 1H).

MS (ESI⁺) m/z: 549 (MH⁺).

HRMS (ESI⁺) for C₂₈H₃₀FN₆O₅ (MH⁺): calcd, 549.22617. found, 549.22581.

### Example 293

The following compound was prepared consistent with the methods described herein.

### 6-((1-(2-(3-Fluoro-6-(4-hydroxypiperidin-1-yl)-1,5-naphthyridin-4-yl)ethyl)-2-oxabicyclo[2.2.2]octan-4-ylamino)methyl)-2H-pyrido[3,2-b][1,4]oxazin-3(4H)-one

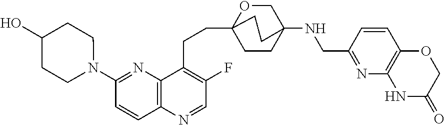 [SMILES: O=C1COc2ccc(CNC34CCC(CCc5c(F)cnc6ccc(N7CCC(O)CC7)nc56)(CC3)OC4)nc2N1]

¹H NMR (DMSO-d₆) δ 1.30-1.45 (m, 2H), 1.53-1.74 (m, 8H), 1.75-1.93 (m, 5H), 2.95-3.05 (m, 2H), 3.31-3.41 (m, 2H), 3.58 (s, 2H), 3.62 (d, J=5.5 Hz, 2H), 3.73-3.83 (m, 1H), 4.18-4.28 (m, 2H), 4.59 (s, 2H), 4.72 (d, J=4.3 Hz, 1H), 7.01 (d, J=7.9 Hz, 1H), 7.27 (d, J=7.9 Hz, 1H), 7.42 (d, J=9.8 Hz, 1H), 7.99 (d, J=9.2 Hz, 1H), 8.46 (s, 1H), 11.15 (s, 1H).

MS (ESI⁺) m/z: 563 (MH⁺).

HRMS (ESI⁺) for C₃₀H₃₆FN₆O₄ (MH⁺): calcd, 563.27821. found, 563.27904.

### Example 294

The following compound was prepared consistent with the methods described herein.

### (S)-6-((1-(2-(3-Fluoro-6-(3-hydroxypyrrolidin-1-yl)-1,5-naphthyridin-4-yl)ethyl)-2-oxabicyclo[2.2.2]octan-4-ylamino)methyl)-2H-pyrido[3,2-b][1,4]oxazin-3(4H)-one

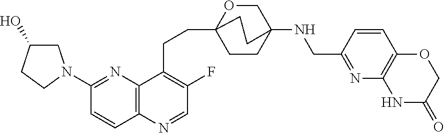 [SMILES: O=C1COc2ccc(CNC34CCC(CCc5c(F)cnc6ccc(N7CC[C@H](O)C7)nc56)(CC3)OC4)nc2N1]

¹H NMR (DMSO-d₆) δ 1.52-1.77 (m, 8H), 1.79-2.14 (m, 5H), 2.95-3.06 (m, 2H), 3.51-3.72 (m, 8H), 4.44 (br, 1H), 4.59 (s, 2H), 5.01-5.05 (m, 1H), 7.01 (d, J=8.0 Hz, 1H), 7.04 (d, J=9.2 Hz, 1H), 7.28 (d, J=8.0 Hz, 1H), 7.99 (d, J=9.2 Hz, 1H), 8.42 (s, 1H), 11.15 (s, 1H).

MS (ESI⁺) m/z: 549 (MH⁺).

HRMS (ESI⁺) for C₂₉H₃₄FN₆O₄ (MH⁺): calcd, 549.26256. found, 549.26323.

### Example 295

The following compound was prepared consistent with the methods described herein.

### 6-((1-(2-(3-Fluoro-6-(3-hydroxyazetidin-1-yl)-1,5-naphthyridin-4-yl)ethyl)-2-oxabicyclo[2.2.2]octan-4-ylamino)methyl)-2H-pyrido[3,2-b][1,4]oxazin-3(4H)-one

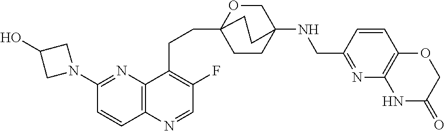 [SMILES: O=C1COc2ccc(CNC34CCC(CCc5c(F)cnc6ccc(N7CC(O)C7)nc56)(CC3)OC4)nc2N1]

¹H NMR (DMSO-d₆) δ 1.52-1.78 (m, 8H), 1.78-1.92 (m, 3H), 2.92-3.05 (m, 2H), 3.58 (s, 2H), 3.63 (d, J=6.1 Hz, 2H), 3.85 (dd, J=9.2, 4.3 Hz, 2H), 4.29-4.36 (m, 2H), 4.55-4.67 (m, 3H), 5.74 (d, J=6.1 Hz, 1H), 6.89 (d, J=9.2, 1H), 7.00 (d, J=7.9 Hz, 1H), 7.27 (d, J=7.9 Hz, 1H), 8.00 (d, J=9.2 Hz, 1H), 8.47 (s, 1H), 11.15 (s, 1H).

MS (ESI⁺) m/z: 535 (MH⁺).

HRMS (ESI⁺) for C₂₈H₃₂FN₆O₄ (MH⁺): calcd, 535.24691. found, 535.24623.

### Example 296

The following compound was prepared consistent with the methods described herein.

### (R)-6-((1-(2-(3-Fluoro-6-(3-hydroxypyrrolidin-1-yl)-1,5-naphthyridin-4-yl)ethyl)-2-oxabicyclo[2.2.2]octan-4-ylamino)methyl)-2H-pyrido[3,2-b][1,4]oxazin-3(4H)-one

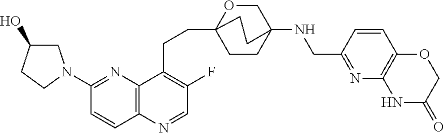 [SMILES: O=C1COc2ccc(CNC34CCC(CCc5c(F)cnc6ccc(N7CC[C@@H](O)C7)nc56)(CC3)OC4)nc2N1]

¹H NMR (DMSO-d₆) δ 1.52-1.77 (m, 8H), 1.79-2.14 (m, 5H), 2.95-3.06 (m, 2H), 3.51-3.72 (m, 8H), 4.44 (br, 1H), 4.59 (s, 2H), 5.01-5.05 (m, 1H), 7.01 (d, J=8.0 Hz, 1H), 7.04 (d, J=9.2 Hz, 1H), 7.28 (d, J=8.0 Hz, 1H), 7.99 (d, J=9.2 Hz, 1H), 8.42 (s, 1H), 11.15 (s, 1H).

MS (ESI⁺) m/z: 549 (MH⁺).

HRMS (ESI⁺) for C₂₉H₃₄FN₆O₄ (MH⁺): calcd, 549.26256. found, 549.26312.

### Example 297

The following compound was prepared consistent with the methods described herein.

### 6-((1-(2-(3-Fluoro-6-(2-hydroxyethylamino)-1,5-naphthyridin-4-yl)ethyl)-2-oxabicyclo[2.2.2]octan-4-ylamino)methyl)-2H-pyrido[3,2-b][1,4]oxazin-3(4H)-one

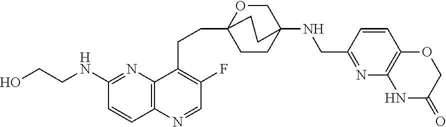 [SMILES: O=C1COc2ccc(CNC34CCC(CCc5c(F)cnc6ccc(NCCO)nc56)(CC3)OC4)nc2N1]

¹H NMR (DMSO-d₆) δ 1.54-1.76 (m, 8H), 1.77-1.91 (m, 3H), 2.92-3.04 (m, 2H), 3.41-3.53 (m, 2H), 3.56-3.67 (m, 6H), 4.59 (s, 2H), 4.70 (t, J=5.5 Hz, 1H), 6.97 (d, J=9.2 Hz, 1H), 7.00 (d, J=7.9 Hz, 1H), 7.28 (d, J=7.9 Hz, 1H), 7.50 (br, 1H), 7.84 (d, J=9.2 Hz, 1H), 8.38 (s, 1H), 11.15 (s, 1H).

MS (ESI⁺) m/z: 523 (MH⁺).

HRMS (ESI⁺) for C₂₇H₃₂FN₆O₄ (MH⁺): calcd, 523.24691. found, 523.24709.

### Example 298

The following compound was prepared consistent with the methods described herein.

### 2-(8-(2-(4-((2,3-Dihydro-[1,4]dioxino[2,3-c]pyridin-7-yl)methylamino)-2-oxabicyclo[2.2.2]octan-1-yl)ethyl)-7-fluoro-1,5-naphthyridin-2-yloxy)-N-methylacetamide

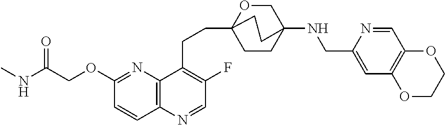 [SMILES: CNC(=O)COc1ccc2ncc(F)c(CCC34CCC(NCc5cc6c(cn5)OCCO6)(CC3)CO4)c2n1]

¹H NMR (DMSO-d₆) δ 1.50-1.75 (m, 8H), 1.76-2.02 (m, 3H), 2.62 (d, J=4.9 Hz, 3H), 2.97-3.09 (m, 2H), 3.55 (s, 2H), 3.61 (d, J=6.7 Hz, 1H), 4.24-4.28 (m, 2H), 4.30-4.35 (m, 2H), 4.89 (s, 2H), 6.92 (s, 1H), 7.29 (d, J=9.2 Hz, 1H), 7.96-8.03 (m, 2H), 8.31 (d, J=9.2 Hz, 1H), 8.76 (s, 1H).

MS (ESI⁺) m/z: 528 (MH⁺).

HRMS (ESI⁺) for C₂₈H₃₃FN₅O₅ (MH⁺): calcd, 538.24657. found, 538.24639.

It will be appreciated that various of the above-discussed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

1. A compound of Formula (Ib): 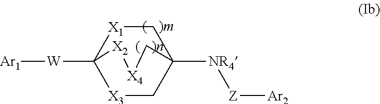
wherein:
X₁, X₂, and X₃ are independently CR₁R₂, O, S, S═O, SO₂ or NR₀;
X₄ is CR₁R₂, O, S, S═O, SO₂, NR₀, or is absent;
with the provisos that if X₂ is O, S, S═O, SO₂ or NR₀, then X₄ is CR₁R₂, if X₄ is O, S, S═O, SO₂ or NR₀, then X₂ is CR₁R₂, and no more than two of X₁, X₂, X₃ and X₄ are O, S, S═O, SO₂ or NR₀;
m is 1, 2, or 3;
n is 0, 1, or 2;
W is C(═O), —CR₁R₂—, —CH═CH—, —C≡C—, —CR₁R₂—CR₁′R₂′—, —O—CR₁R₂—, —O—CR₁R₂—CR₁′R₂′,
—NR₄—CR₁R₂—, or a group of the following structure: 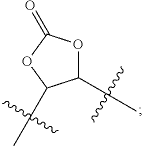 [SMILES: *C1OC(=O)OC1*]
R₀ is H, (C₁₋₆)alkyl, acyl or sulfonyl;
each R1, R2, R1′, and R2′ is independently H, (C₁₋₆)alkyl, (C₁₋₆)hydroxyalkyl, —CO₂R₃, —CONR₄R₅, halogen, OR₃, CF₃, aryl, heteroaryl or NHR₄;
with the proviso that R₁ is not OR₃ or NHR₄ when R₂ is OR₃ or NHR₄, and R₁′ is not OR₃ or NHR₄ when R₂′ is OR₃ or NHR₄;
wherein R₁ and R₂, or R₁′ and R₂′ on the same carbon together may form ═O or ═NOR₄;
R₃ is H, (C₁₋₆)alkyl, hydroxy(C₁₋₆)alkyl, or CF₃;
R₄, R₄′ and R₅ are independently H, (C₁₋₆)alkyl, or CO₂R₃;
Z is CH₂, C(═O), CH₂—CH═CH, CH₂—CH₂—O, or SO₂;
Ar₁ is a group having one of the following structures: 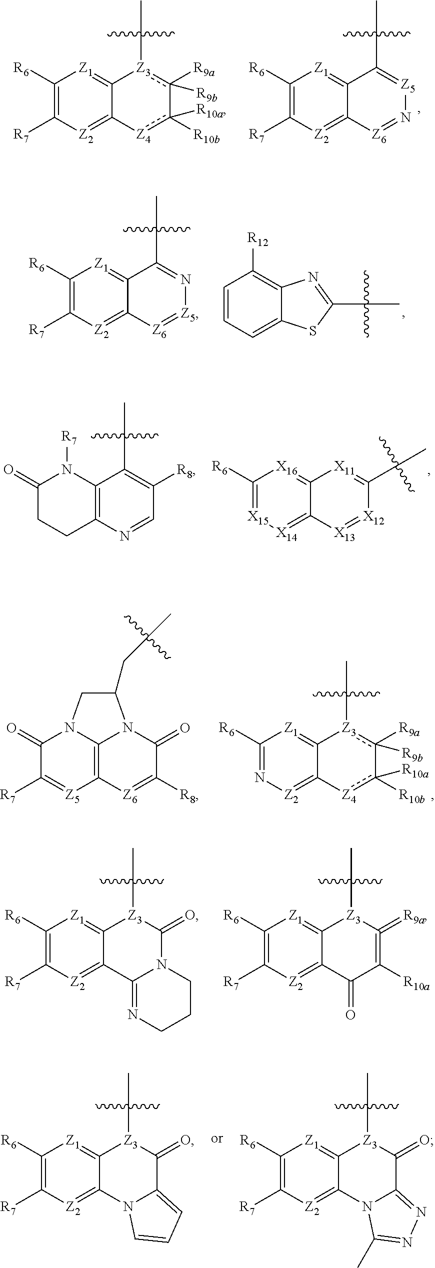
Z₁ is CR₁ₐ or N;
Z₂, Z₅ and Z₆ are independently CR_{1b}, or N;
Z₃ is C or N;
wherein Z₃ is not N if the [symbol] bond to which it is attached is a double bond;
Z₄ is CR₁₁ₐR_{11b}, N, CR₁₁ₐ, NR₁₁ₐ, or O;
wherein Z₄ is not O, NR₁₁ₐ or CR₁₁ₐR_{11b} if the [symbol] bond to which it is attached is a double bond;
X₁₁, X₁₃, X₁₄ and X₁₆ are independently N or CR₁ₐ;
wherein at least one of X₁₁, X₁₃, X₁₄ and X₁₆ is N;
X₁₂ is CH, C—(C₁₋₆)alkyl, C—(C₁₋₆)alkoxy, C-halo, or C—COOH;
X₁₅ is CH, C—(C₁₋₆)alkyl or C-halo;
R₆ is H; OH; NR₁₃R₁₄; (C₁₋₆)alkyl; C(O)OR₁₃; halo; CF₃; cyano; allyloxy; —R₁₅COOR₁₄; —OR₁₅COOR₁₄; —OR₁₅CONR₁₃R₁₄; (C₁₋₆)alkoxy, (C₃₋₆)cycloalkoxy, (C₃₋₆)heterocycleoxy, (C₃₋₁₀)cycloalkylalkoxy, or (C₃₋₁₀)heterocycloalkoxy which are optionally substituted with 1 to 3 substituents selected from NR₁₃R₁₄, CONR₁₃R₁₄, OH, halo, CF₃, COOR₁₄, cyano, oxo, (C₁₋₆)alkyl, or (C₁₋₆)alkoxy; S(O)₂R₁₃ optionally substituted with a (C₁₋₆)alkyl; or 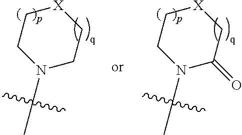
wherein X is CR_{1c}, O, S or SO₂;
each p and q is 0, 1, or 2, with the proviso that if X is O or S, both p and q cannot be 0;
each R₇ and R₈ is independently H, halo, OH, (C₁₋₆)alkoxy, NR₁₃R₁₄, CF₃, or cyano;
R₉ₐ is H, halo, OH, (C₁₋₆)alkoxy, NH₂, or cyano; R_{9b} is absent; and the [symbol] bond attached to Z₃ is a double bond; or
R₉ₐ and R_{9b} together form oxo; and the [symbol] bond attached to Z₃ is a single bond;
R₁₀ₐ is H or (C₁₋₆)alkyl; R_{10b} is absent; and the [symbol] bond attached to Z₄ is a double bond; or
R₁₀ₐ and R_{10b} together form oxo; and the [symbol] bond attached to Z₄ is a single bond;
R₁₁ₐ is H or (C₁₋₆)alkyl; and R_{11b} is absent; and the [symbol] bond attached to Z₄ is a double bond or Z₄ is NR₁₁ₐ; or
R₁₁ₐ and R_{11b} together form oxo; and the [symbol] bond attached to Z₄ is a single bond;
or R₁₀ₐ and R₁₁ₐ together with the atoms to which they are attached form a 5-membered saturated, unsaturated or aromatic ring having 0 to 3 N and optionally substituted with a (C₁₋₆)alkyl, wherein R_{10b} and R_{11b} are H or absent, depending on valence;
each R₁₂, R₁₃ and R₁₄ is independently H, (C₁₋₆)alkyl, or (C₁₋₆)hydroxyalkyl;
each R₁₅ is independently (C₁-C₆)alkylene or (C₂-C₆)alkenylene with the proviso that when R₆ is —OR₁₅COOR₁₄, R₁₅ is not C₂alkenylene;
R₁ₐ is H, OH, (C₁₋₆)alkoxy, cyano, or halo;
R_{1b} is H, (C₁₋₆)alkenyl, (C₁₋₆)alkoxy, halo, cyano, or C(O)OR₁₃;
R_{1c} is H, OH, halo or (C₁₋₆)alkyl;
Ar₂ is
(i) C₃-C₆-cycloalkyl, optionally substituted with —OH, halo, cyano, NR₁₃R₁₄ or (C₁₋₆)alkyl;
(ii) aryl, wherein aryl is phenyl or naphthyl optionally substituted with 1 to 3 substituents selected from OH, halo, (C₁₋₆)alkoxy, halo(C₁₋₆)alkoxy and (C₁₋₆)alkyl;
(iii) a heterocyclyl, wherein the heterocyclyl is a 5- to 6-membered non-aromatic or aromatic ring having 1 or 2 heteroatoms selected from N, O or S optionally substituted with 1 to 3 substitutents selected from OH, halo, cyano, (C₁₋₆)alkoxy, (C₁₋₆)alkyl, NR₁₃R₁₄ and a 5- to 6-membered aromatic or non-aromatic ring having 1 or 2 heteroatoms selected from N, O or S; wherein (C₁₋₆)alkoxy or (C₁₋₆)alkyl are optionally substituted with 1 or 2 halo; or
(iv) a group having one of the following structures: 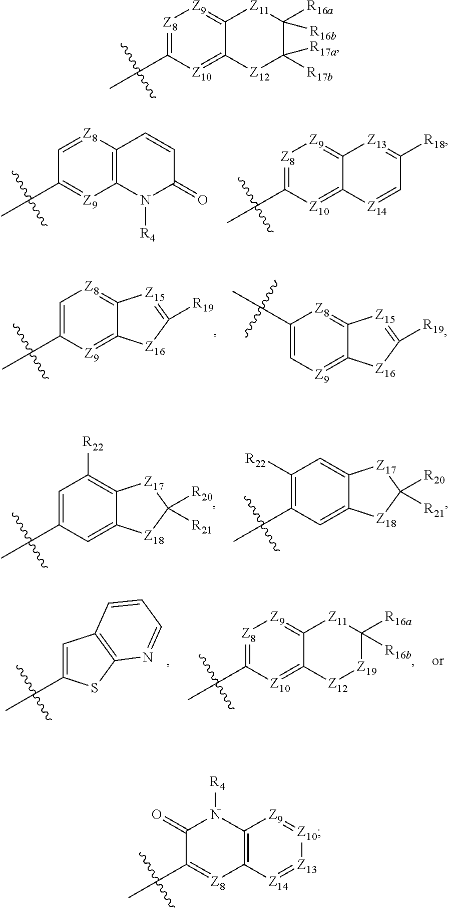
each Z₈, Z₉ and Z₁₀ is independently CR₁ₐ, CR_{1b} or N;
Z₁₁ and Z₁₂ are each independently CR₁ₐR_{1b}, NR₄, O, SO₂ or S;
Z₁₃ and Z₁₄ are each independently CR₁ₐ or N;
Z₁₅ is CR₁ₐ or N;
Z₁₆ is CR₁ₐR_{1b} or NH;
each Z₁₇ and Z₁₈ is independently NR₄ or O;
Z₁₉ is SO₂;
each R₁₆ₐ and R_{16b} is independently H or CH₃;
or R₁₆ₐ and R_{16b} together form oxo;
each R₁₇ₐ and R_{1b} is H;
or R₁₇ₐ and R_{17b} together form oxo or ═NOR₃;
R₁₈ is H or (C₁₋₆)alkoxy;
R₁₉ is H or halo;
each R₂₀, R₂₁ and R₂₂ is independently H or halo;
or R₂₀ and R₂₁ together form oxo;
or a pharmaceutically acceptable salt thereof.

2. A compound of Formula (I): 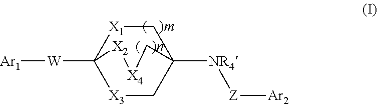
wherein:
X₁, X₂, and X₃ are independently CR₁R₂, O, S, S═O, SO₂ or NR₀;
X₄ is CR₁R₂, O, S, S═P, SO₂, NR₀, or is absent;
with the provisos that if X₂ is O, S, S═O, SO₂ or NR₀, then X₄ is CR₁R₂, if X₄ is O, S, S═O, SO₂ or NR₀, then X₂ is CR₁R₂, and no more than two of X₁, X₂, X₃ and X₄ are O, S, S═O, SO₂ or NR₀;
m is 1, 2, or 3;
n is 0, 1, or 2;
W is C(═O), —CR₁R₂—, —CH═CH—, —C≡C—, —CR₁R₂—CR₁′R₂′—, —O—CR₁R₂—, —NR₄—CR₁R₂—, or a group of the following structure: 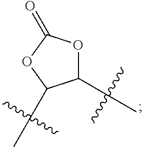 [SMILES: *C1OC(=O)OC1*]
R₀ is H, (C₁₋₆)alkyl, acyl or sulfonyl;
each R₁, R₂, R₁′, and R₂′ is independently H, (C₁₋₆)alkyl, (C₁₋₆)hydroxyalkyl, —CO₂R₃, —CONR₄R₅, halogen, OR₃, CF₃, aryl, heteroaryl or NHR₄;
with the proviso that R₁ is not OR₃ or NHR₄ when R₂ is OR₃ or NHR₄, and R₁′ is not OR₃ or NHR₄ when R₂′ is OR₃ or NHR₄;
wherein R₁ and R₂, or R₁′ and R₂′ on the same carbon together may form ═O or ═NOR₄;
R₃ is H, (C₁₋₆)alkyl, hydroxyl(C₁₋₆)alkyl or CF₃;
R₄, R₄′ and R₅ are independently H, (C₁₋₆)alkyl, or CO₂R₃;
Z is CH₂, C(═O), CH₂—CH═CH, or SO₂;
Ar₁ is a group having one of the following structures: 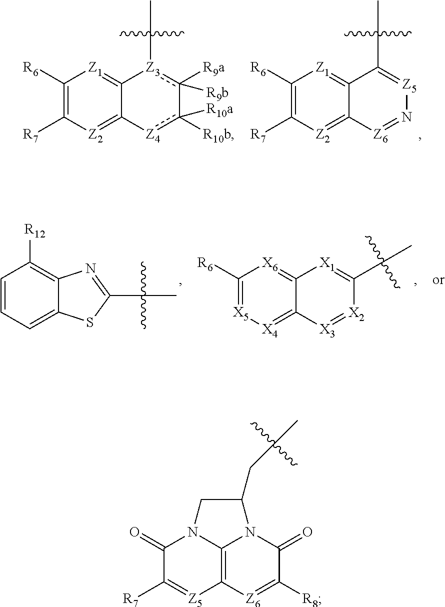
Z₁ is CR₁ₐ or N;
Z₂, Z₅ and Z₆ are independently CR_{1b}, or N;
Z₃ is C or N if the [symbol] bond to which it is attached is a single bond; or
Z₃ is C if the [symbol] bond to which it is attached is a double bond;
Z₄ is CR₁₁ₐR_{11b}, NR₁₁ₐ, or O if the [symbol] bond to which it is attached is a single bond; or
Z₄ is CR₁₁ₐ or N if the [symbol] bond to which it is attached is a double bond;
X₁, X₃, X₄ and X₆ are independently N or CR₁ₐ;
wherein at least one of X₁, X₃, X₄ and X₆ is N;
X₂ is CH, C—(C₁₋₆)alkyl, C—(C₁₋₆)alkoxy, C-halo, or C—COOH;
X₅ is CH, C—(C₁₋₆)alkyl or C-halo;
R₆ is H; OH; NR₁₃R₁₄; (C₁₋₆)alkyl; C(O)OR₁₃; halo; CF₃; cyano; allyloxy; —R₁₅COOR₁₄; —OR₁₅COOR₁₄; (C₁₋₆)alkoxy, (C₃₋₆)cycloalkoxy, (C₃₋₆)heterocycleoxy, (C₃₋₆)cycloalkylalkoxy, or (C₃₋₆)heterocycloalkoxy which are optionally substituted with NR₁₃R₁₄, OH, CF₃, COOR₁₄, cyano, oxo, (C₁₋₆)alkyl or (C₁₋₆)alkoxy; S(O)₂R₁₃ optionally substituted with a (C₁₋₆)alkyl; or 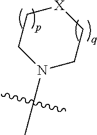
wherein X is CR_{1c}, O or S;
each p and q is 0, 1, or 2, with the proviso that if X is O or S, both p and q cannot be 0;
each R₇ and R₈ is independently H, halo, OH, (C₁₋₆)alkoxy, NR₁₃R₁₄, CF₃, or cyano;
R₉ₐ is H, halo, OH, (C₁₋₆)alkoxy, NH₂, or cyano; R_{9b} is absent; and the [symbol] bond attached to Z₃ is a double bond; or
R₉ₐ and R_{9b} together form oxo; and the [symbol] bond attached to Z₃ is a single bond;
R₁₀ₐ is H or (C₁₋₆)alkyl; R_{10b} is absent; and the [symbol] bond attached to Z₄ is a double bond; or
R₁₀ₐ and R_{10b} together form oxo; and the [symbol] bond attached to Z₄ is a single bond or Z₄ is NR₁₁ₐ;
R₁₁ₐ is H or (C₁₋₆)alkyl; and R_{11b} is absent; and the [symbol] bond attached to Z₄ is a double bond; or
R₁₁ₐ and R_{11b} together form oxo; and the [symbol] bond attached to Z₄ is a single bond;
or R₁₀ₐ and R₁₁ₐ together with the atoms to which they are attached form a 5-membered saturated, unsaturated or aromatic ring having 0 to 3 N and optionally substituted with a (C₁₋₆)alkyl, wherein R_{10b} and R_{11b} are H or absent, depending on valence;
each R₁₂, R₁₃ and R₁₄ is independently H or (C₁₋₆)alkyl;
each R₁₅ is independently (C₁-C₆)alkylene or (C₂-C₆)alkenylene with the proviso that when R₆ is —OR₁₅COOR₁₄, R₁₅ is not C₂alkenylene;
R₁ₐ is H, OH, (C₁₋₆)alkoxy, cyano, or halo;
R_{1b} is H, (C₁₋₆)alkoxy, halo, cyano, or C(O)OR₁₃;
R_{1c} is H, halo or (C₁₋₆)alkyl;
Ar₂ is
(i) C₃-C₆-cycloalkyl, optionally substituted with —OH, halo, cyano, NR₁₃R₁₄ or (C₁₋₆)alkyl;
(ii) aryl, wherein aryl is phenyl or naphthyl optionally substituted with 1 to 3 substituents selected from OH, halo, (C₁₋₆)alkoxy, halo(C₁₋₆)alkoxy and (C₁₋₆)alkyl;
(iii) a heterocyclyl, wherein the heterocyclyl is a 5- to 6-membered non-aromatic or aromatic ring having 1 or 2 heteroatoms selected from N, O or S optionally substituted with 1 to 3 substitutents selected from OH, halo, cyano, (C₁₋₆)alkoxy, (C₁₋₆)alkyl, NR₁₃R₁₄ and a 5- to 6-membered aromatic or non-aromatic ring having 1 or 2 heteroatoms selected from N, O or S; wherein (C₁₋₆)alkoxy or (C₁₋₆)alkyl optionally substituted with 1 or 2 halo; or
(iv) a group having one of the following structures: 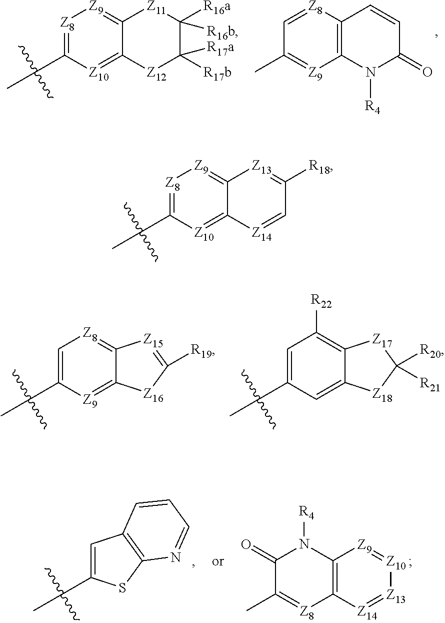
each Z₈, Z₉ and Z₁₀ is independently CR₁ₐ or N;
Z₁₁ and Z₁₂ are each independently CR₁ₐR_{1b}, NR₄, O, or S;
Z₁₃ and Z₁₄ are each independently CR₁ₐ or N;
Z₁₅ is CR₁ₐ or N;
Z₁₆ is CR₁ₐR_{1b} or NH;
each Z₁₇ and Z₁₈ is independently NR₄ or O;
each R₁₆ₐ and R_{16b} is independently H or CH₃;
or R₁₆ₐ and R_{16b} together form oxo;
each R₁₇ₐ and R_{1b} is H;
or R₁₇ₐ and R_{1b} together form oxo or ═NOR₃;
R₁₈ is H or (C₁₋₆)alkoxy;
R₁₉ is H or halo;
each R₂₀, R₂₁ and R₂₂ is independently H or halo;
or a pharmaceutically acceptable salt thereof.

3. A compound of Formula (Ia): 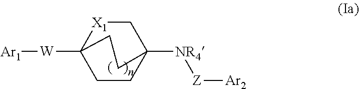
wherein:
X₁ is CH₂, O, or NR₀;
n is 0 or 1;
W is C(═O), —CH═CH—, —C≡C—, —CR₁R₂—CR₁R₂—, —O—CR₁R₂—CR₁R₂—, —CH₂—CR₁R₂—, —CR₁R₂—CH₂—, —O—CR₁R₂—, —NHR₄—CR₁R₂—, or a group of the following structure: 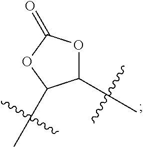 [SMILES: *C1OC(=O)OC1*]
each R₁ and R₂ is independently H, halo, (C₁₋₆)alkyl, OR₃, or NHR₄, wherein only one of R₁ or R₂ on the same carbon is OR₃ or NHR₄;
or R₁ and R₂ on the same carbon together form ═O or ═NOR₃;
R₃ is H or (C₁₋₆)alkyl;
Ar₁ is a group having one of the following structures: 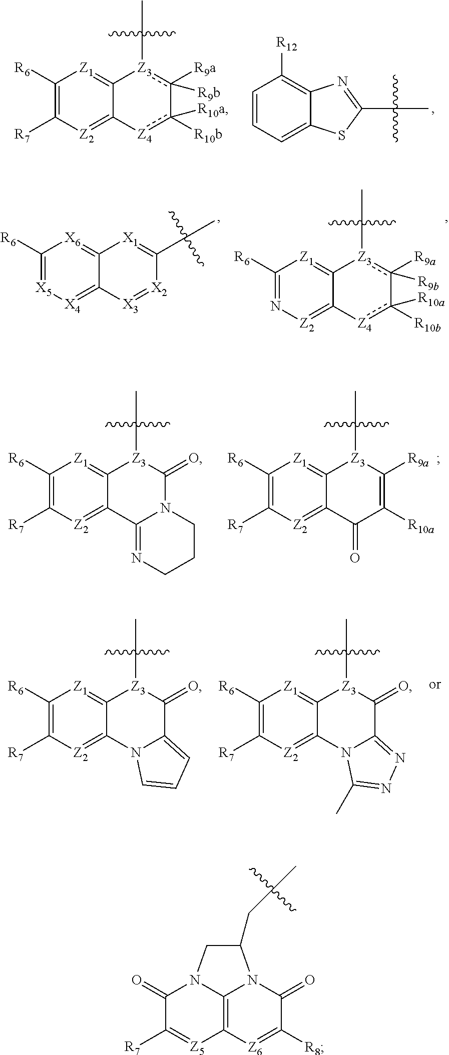
and all other variables are as defined in claim 1;
or a pharmaceutically acceptable salt thereof.

4. The compound of claim 1, wherein
X₁ is CH₂ or O;
n is 1;
W is —CH═CH—, —C≡C—, —CR₁R₂—CR₁R₂—, —CH₂—CR₁R₂—, —CR₁R₂—CH₂—, or —O—CH₂—;
each R₁ and R₂ is independently H, (C₁₋₆)alkyl or OH, wherein only one of R₁ or R₂ on the same carbon is OH;
R₄′ is H or (C₁₋₆)alkyl;
Z is CH₂ or CH₂—CH═CH;
Ar₁ is a group of the following structure: 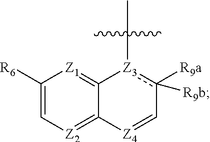
Z₄ is CR₁₁ₐ or N;
and no more than three of Z₁, Z₂, Z₃, and Z₄ are N;
R₆ is OH; (C₁₋₆)alkyl; halo; CF₃; cyano; (C₁₋₆)alkoxy, (C₃₋₆)cycloalkoxy, (C₃₋₆)heterocycleoxy, (C₃₋₆)cycloalkylalkoxy, or (C₃₋₆)heterocycloalkoxy which are optionally substituted with NR₁₃R₁₄, OH, CF₃, COOR₁₄, cyano, oxo or (C₁₋₆)alkoxy;
R₉ₐ is H, F, Cl, OH, (C₁₋₆)alkoxy, or cyano; R_{9b} is absent; and the [symbol] bond attached to Z₃ is a double bond; or
R₉ₐ and R_{9b} together form oxo; and the [symbol] bond attached to Z₃ is a single bond;
R₁₁ₐ is H or (C₁₋₆)alkyl;
R₁ₐ is H, halo or (C₁₋₆)alkoxy;
R_{1b} is H, (C₁₋₆)alkyl, halo, or (C₁₋₆)alkoxy;
Ar₂ is selected from
aryl, wherein aryl is phenyl optionally substituted with 1 or 2 halo;
or a group of the following structure: 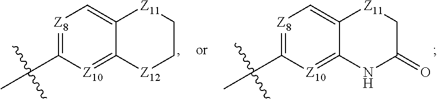
Z₁₀ is CH or N;
Z₁₁ and Z₁₂ are CR₁ₐR_{1b}, N—(C₁₋₆)alkyl, O or S;
and all other variables are as defined in claim 1;
or a pharmaceutically acceptable salt thereof.

5. The compound of claim 1, wherein
X₁ is CH₂ or O;
n is 1;
W is —CH₂—CH₂—, —CH═CH—, —C≡C—, —CH₂—CHOH—, —CHOH—CH₂, —CH₂—C(CH₃)OH, or —O—CH₂;
Z is CH₂ or —CH₂—CH═CH—;
Ar₁ is a group having one of the following structures: 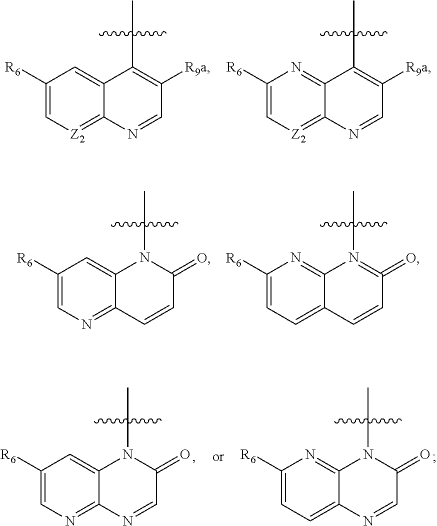
Z₂ is CR_{1b};
R₆ is (C₁₋₆)alkyl; halo; cyano; or (C₁₋₆)alkoxy, (C₃₋₆)cycloalkylalkoxy, or (C₃₋₆)heterocycloalkoxy which are optionally substituted with OH, COOR₁₄, cyano, or oxo;
R₉ₐ is H, F, Cl, OH, or cyano;
R_{1b} is H, F, Cl, or (C₁₋₆)alkyl;
Ar₂ is a group having one of the following structures: 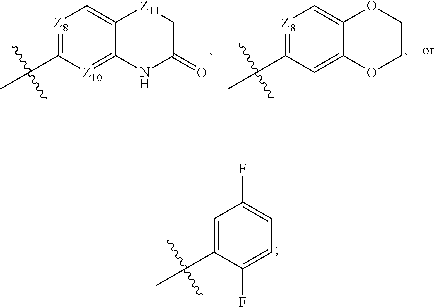
Z₈ and Z₁₀ are independently CR₁ₐ or N;
R₁ₐ is H, F, Cl, or (C₁₋₆)alkoxy;
Z₁₁ is O or S;
and the other variables are as defined in claim 1;
or a pharmaceutically acceptable salt thereof.

6. The compound of claim 1, wherein Z is —CH₂—CHOH—.

7. The compound of claim 1 wherein Ar₂ is

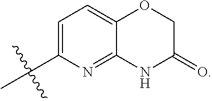 [SMILES: *c1ccc2c(n1)NC(=O)CO2]

8. The compound of claim 1, wherein Ar₁ is

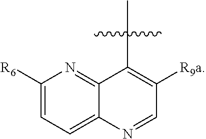

9. The compound of claim 1, wherein X₁ is O.

10. The compound of claim 1, wherein each R₁, R₂, R₁′, and R₂′ is independently H, OH, (C₁₋₆)alkyl, or (C₁₋₆)hydroxyalkyl.

11. The compound of claim 1, wherein Ar₁ is

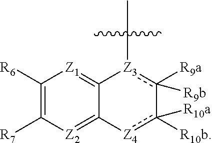

12. The compound of claim 1, wherein Ar₁ is

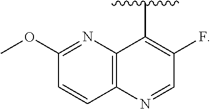 [SMILES: *c1c(F)cnc2ccc(OC)nc12]

13. The compound of claim 1, wherein Ar₂ is

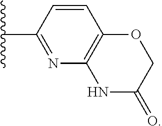 [SMILES: *c1ccc2c(n1)NC(=O)CO2]

14. The compound of claim 1, wherein the compound is:
(E)-6-((1-(2-(3-Fluoro-6-methoxy-1,5-naphthyridin-4-yl)vinyl)-2-oxabicyclo[2.2.2]octan-4-ylamino)methyl)-2H-pyrido[3,2-b][1,4]oxazin-3(4H)-one;
6-((1-(2-(3-Chloro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one;
N-((2,3-Dihydrobenzo[b][1,4]dioxin-6-yl)methyl)-1-(2-(3-fluoro-6-methoxy-1,5-naphthyridin-4-yl)ethyl)-2-oxabicyclo[2.2.2]octan-4-amine;
7-Chloro-6-(((1-(2-(3-fluoro-6-methoxy-1,5-naphthyridin-4-yl)ethyl)-2-oxabicyclo[2.2.2]octan-4-yl)amino)methyl)-2H-pyrido[3,2-b][1,4]oxazin-3(4H)-one;
6-((4-(2-(3-Fluoro-6-methoxy-1,5-naphthyridin-4-yl)ethyl)bicyclo[2.2.2]octan-1-ylamino)methyl)-2H-pyrido[3,2-b][1,4]oxazin-3(4H)-one;
6-((1-(2-(3-Fluoro-6-methoxy-1,5-naphthyridin-4-yl)ethyl)-2-oxabicyclo[2.2.2]octan-4-ylamino)methyl)-2H-pyrido[3,2-b][1,4]oxazin-3(4H)-one;
6-Methoxy-4-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-3-carbonitrile;
6-(((1-(1-Hydroxy-2-(7-methoxy-2-oxo-1,5-naphthyridin-1(2H)-yl)ethyl)-2-oxabicyclo[2.2.2]octan-4-yl)amino)methyl)-2H-pyrido[3,2-b][1,4]oxazin-3(4H)-one;
6-(((1-(2-(7-Methoxy-2-oxopyrido[2,3-b]pyrazin-1(2H)-yl)ethyl)-2-oxabicyclo[2.2.2]octan-4-yl)amino)methyl)-2H-pyrido[3,2-b][1,4]oxazin-3(4H)-one;
6-((1-(2-(7-Methoxy-2-oxo-1,8-naphthyridin-1(2H)-yl)ethyl)-2-oxabicyclo[2.2.2]octan-4-ylamino)methyl)-2H-pyrido[3,2-b][1,4]oxazin-3(4H)-one;
6-((1-(2-(3,8-difluoro-6-methoxyquinolin-4-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-methoxy-1,5-naphthyridin-4-yl)ethyl)-2-oxabicyclo[2.2.2]octan-4-ylamino)methyl)-2H-pyrido[3,2-b][1,4]thiazin-3(4H)-one;
7-Fluoro-6-(((1-(2-(3-fluoro-6-methoxy-1,5-naphthyridin-4-yl)ethyl)-2-oxabicyclo[2.2.2]octan-4-yl)amino)methyl)-2H-pyrido[3,2-b][1,4]oxazin-3(4H)-one;
(E)-6-((4-(2-(3-Chloro-6-methoxy-1,5-naphthyridin-4-yl)vinyl)bicyclo[2.2.2]octan-1-ylamino)methyl)-2H-pyrido[3,2-b][1,4]oxazin-3(4H)-one;
6-(((1-(2-(7-Methoxy-2-oxo-1,5-naphthyridin-1(2H)-yl)ethyl)-2-oxabicyclo[2.2.2]octan-4-yl)amino)methyl)-2H-pyrido[3,2-b][1,4]oxazin-3(4H)-one;
6-((1-(2-(3-Fluoro-6-methoxyquinolin-4-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-(3-hydroxypropoxy)-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(3-fluoro-6-methoxy-8-methyl-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(3-fluoro-6-methoxy-8-methyl-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(6-Methoxy-1,5-naphthyridin-4-yl)ethyl)-2-oxabicyclo[2.2.2]octan-4-ylamino)methyl)-2H-pyrido[3,2-b][1,4]oxazin-3(4H)-one;
6-((4-((3-Chloro-6-methoxy-1,5-naphthyridin-4-yloxy)methyl)bicyclo[2.2.2]octan-1-ylamino)methyl)-2H-pyrido[3,2-b][1,4]oxazin-3(4H)-one;
6-((1-(2-(7-Methoxy-2-oxo-1,8-naphthyridin-1(2H)-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-(2-hydroxyethoxy)-1,5-naphthyridin-4-yl)ethyl)-2-oxabicyclo[2.2.2]octan-4-ylamino)methyl)-2H-pyrido[3,2-b][1,4]oxazin-3(4H)-one;
6-((4-(2-(3-Chloro-6-methoxy-1,5-naphthyridin-4-yl)ethyl)bicyclo[2.2.2]octan-1-ylamino)methyl)-2H-pyrido[3,2-b][1,4]oxazin-3(4H)-one;
6-((1-(2-(6-Methoxy-1,5-naphthyridin-4-yl)ethyl)-2-oxabicyclo[2.2.2]octan-4-ylamino)methyl)-2H-pyrido[3,2-b][1,4]oxazin-3(4H)-one;
6-((4-((3-Chloro-6-methoxy-1,5-naphthyridin-4-yl)ethynyl)bicyclo[2.2.2]octan-1-ylamino)methyl)-2H-pyrido[3,2-b][1,4]oxazin-3(4H)-one;
6-((4-(2-(6-Methoxy-1,5-naphthyridin-4-yl)ethyl)bicyclo[2.2.2]octan-1-ylamino)methyl)-2H-pyrido[3,2-b][1,4]oxazin-3(4H)-one;
6-((4-(2-(3-Chloro-6-methoxy-1,5-naphthyridin-4-yl)-2-hydroxyethyl)bicyclo[2.2.2]octan-1-ylamino)methyl)-2H-pyrido[3,2-b][1,4]oxazin-3(4H)-one;
6-((4-(2-(3-Chloro-6-methoxy-1,5-naphthyridin-4-yl)-1-hydroxyethyl)bicyclo[2.2.2]octan-1-ylamino)methyl)-2H-pyrido[3,2-b][1,4]oxazin-3(4H)-one;
6-(((1-(1-Hydroxy-2-(6-methoxy-3-oxopyrido[2,3-b]pyrazin-4(3H)-yl)ethyl)-2-oxabicyclo[2.2.2]octan-4-yl)amino)methyl)-2H-pyrido[3,2-b][1,4]oxazin-3(4H)-one;
7-((1-(2-(3-Fluoro-6-methoxy-1,5-naphthyridin-4-yl)ethyl)-2-oxabicyclo[2.2.2]octan-4-ylamino)methyl)-1H-pyrido[3,4-b][1,4]oxazin-2(3H)-one;
7-Fluoro-6-((1-(2-(3-fluoro-6-methoxy-1,5-naphthyridin-4-yl)-1-hydroxyethyl)-2-oxabicyclo[2.2.2]octan-4-ylamino)methyl)-2H-pyrido[3,2-b][1,4]oxazin-3(4H)-one;
6-((4-(2-(3-Chloro-6-methoxy-1,5-naphthyridin-4-yl)-1-hydroxyethyl)bicyclo[2.2.2]octan-1-ylamino)methyl)-2H-pyrido[3,2-b][1,4]oxazin-3(4H)-one;
6-(((1-(1-Hydroxy-2-(7-methoxy-2-oxo-1,8-naphthyridin-1(2H)-yl)ethyl)-2-oxabicyclo[2.2.2]octan-4-yl)amino)methyl)-2H-pyrido[3,2-b][1,4]oxazin-3(4H)-one;
6-(((1-(2-(3-Hydroxy-6-methoxy-1,5-naphthyridin-4-yl)ethyl)-2-oxabicyclo[2.2.2]octan-4-yl)amino)methyl)-2H-pyrido[3,2-b][1,4]oxazin-3(4H)-one;
1-(4-((2,3-Dihydro-[1,4]dioxino[2,3-c]pyridin-7-yl)methylamino)-2-oxabicyclo[2.2.2]octan-1-yl)-2-(3-fluoro-6-methoxy-1,5-naphthyridin-4-yl)ethanol;
4-(2-(4-((2,3-Dihydro-[1,4]dioxino[2,3-c]pyridin-7-yl)methylamino)-2-oxabicyclo[2.2.2]octan-1-yl)ethyl)-6-methoxy-1,5-naphthyridine-3-carbonitrile;
6-((1-(2-(3-Fluoro-6-methoxy-1,5-naphthyridin-4-yl)-1-hydroxyethyl)-2-oxabicyclo[2.2.2]octan-4-ylamino)methyl)-2H-pyrido[3,2-b][1,4]oxazin-3(4H)-one;
6-(((1-(1-Hydroxy-2-(6-methoxy-3-oxopyrido[2,3-b]pyrazin-4(3H)-yl)ethyl)-2-oxabicyclo[2.2.2]octan-4-yl)amino)methyl)-2H-pyrido[3,2-b][1,4]oxazin-3(4H)-one;
7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-2-carbonitrile;
4-(2-(4-((2,3-Dihydro-[1,4]dioxino[2,3-c]pyridin-7-yl)methylamino)-2-oxabicyclo[2.2.2]octan-1-yl)ethyl)-6-methoxypyrido[3,2-b]pyrazin-3(4H)-one;
6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridine-3-carbonitrile;
6-(((1-(1-Hydroxy-2-(7-methoxy-2-oxo-1,8-naphthyridin-1(2H)-yl)ethyl)-2-oxabicyclo[2.2.2]octan-4-yl)amino)methyl)-2H-pyrido[3,2-b][1,4]oxazin-3(4H)-one;
6-((4-((3-Chloro-6-methoxy-1,5-naphthyridin-4-yloxy)methyl)bicyclo[2.2.2]octan-1-ylamino)methyl)-2H-pyrido[3,2-b][1,4]oxazin-3(4H)-one;
6-((1-((3-Fluoro-6-methoxy-1,5-naphthyridin-4-yloxy)methyl)-2-oxabicyclo[2.2.2]octan-4-ylamino)methyl)-2H-pyrido[3,2-b][1,4]oxazin-3(4H)-one;
((1-(1-(3-Fluoro-6-methoxy-1,5-naphthyridin-4-yl)-2-hydroxypropan-2-yl)-2-oxabicyclo[2.2.2]octan-4-ylamino)methyl)-2H-pyrido[3,2-b][1,4]oxazin-3(4H)-one;
6-((1-(2-(3-Fluoro-6-methoxy-1,5-naphthyridin-4-yl)-1-hydroxyethyl)-2-oxabicyclo[2.2.2]octan-4-ylamino)methyl)-2H-pyrido[3,2-b][1,4]thiazin-3(4H)-one;
6-((1-(2-(7-Methyl-2-oxo-1,8-naphthyridin-1(2H)-yl)ethyl)-2-oxabicyclo[2.2.2]octan-4-ylamino)methyl)-2H-pyrido[3,2-b][1,4]oxazin-3(4H)-one; and
6-((1-(2-(7-Fluoro-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one;
1-(2-(3-Fluoro-6-methoxy-1,5-naphthyridin-4-yl)-3-hydroxypropyl)-N-((3-oxo-3,4-dihydro-2H-pyrido[3,2-b][1,4]oxazin-6-yl)methyl)-2-oxabicyclo[2.2.2]octan-4-aminium chloride;
sodium 2-(3-fluoro-6-methoxy-1,5-naphthyridin-4-yl)-3-(4-(((3-oxo-3,4-dihydro-2H-pyrido[3,2-b][1,4]oxazin-6-yl)methyl)amino)-2-oxabicyclo[2.2.2]octan-1-yl)propanoate;
7-Chloro-N-(4-(2-(3-fluoro-6-methoxy-1,5-naphthyridin-4-yl)ethyl)bicyclo[2.2.2]octan-1-yl)-3-oxo-3,4-dihydro-2H-pyrido[3,2-b][1,4]thiazine-6-carboxamide;
1-(2-(3-Fluoro-6-methoxy-1,5-naphthyridin-4-yl)ethyl)-N-((7-fluoro-8-methyl-3-oxo-3,4-dihydro-2H-pyrido[3,2-b][1,4]oxazin-6-yl)methyl)-2-oxabicyclo[2.2.2]octan-4-aminium chloride;
N-((7-Ethyl-8-methyl-3-oxo-3,4-dihydro-2H-pyrido[3,2-b][1,4]oxazin-6-yl)methyl)-1-(2-(3-fluoro-6-methoxy-1,5-naphthyridin-4-yl)ethyl)-2-oxabicyclo[2.2.2]octan-4-aminium chloride;
1-(2-(3-Fluoro-6-methoxy-1,5-naphthyridin-4-yl)ethyl)-N-((8-methyl-3-oxo-7-vinyl-3,4-dihydro-2H-pyrido[3,2-b][1,4]oxazin-6-yl)methyl)-2-oxabicyclo[2.2.2]octan-4-aminium chloride;
(R)—N-((2,3-Dihydro-[1,4]dioxino[2,3-c]pyridin-7-yl)methyl)-4-(2-(3-fluoro-6-methoxy-1,5-naphthyridin-4-yl)-1-hydroxyethyl)bicyclo[2.2.2]octan-1-aminium chloride;
(S)—N-((2,3-Dihydro-[1,4]dioxino[2,3-c]pyridin-7-yl)methyl)-4-(2-(3-fluoro-6-methoxy-1,5-naphthyridin-4-yl)-1-hydroxyethyl)bicyclo[2.2.2]octan-1-aminium chloride;
1-(2-(6-Cyano-3-oxo-2H-benzo[b][1,4]oxazin-4(3H)-yl)ethyl)-N-((3-oxo-3,4-dihydro-2H-pyrido[3,2-b][1,4]oxazin-6-yl)methyl)-2-oxabicyclo[2.2.2]octan-4-aminium chloride;
1-(2-(6-Bromo-3-oxo-2H-benzo[b][1,4]oxazin-4(3H)-yl)ethyl)-N-((3-oxo-3,4-dihydro-2H-pyrido[3,2-b][1,4]oxazin-6-yl)methyl)-2-oxabicyclo[2.2.2]octan-4-aminium chloride
(S)-1-(2-(3-Fluoro-6-methoxy-1,5-naphthyridin-4-yl)-1-hydroxyethyl)-N-((3-oxo-3,4-dihydro-2H-benzo[b][1,4]thiazin-6-yl)methyl)-2-oxabicyclo[2.2.2]octan-4-aminium chloride;
(S)—N-((1,1-Dioxido-3-oxo-3,4-dihydro-2H-benzo[b][1,4]thiazin-6-yl)methyl)-1-(2-(3-fluoro-6-methoxy-1,5-naphthyridin-4-yl)-1-hydroxyethyl)-2-oxabicyclo[2.2.2]octan-4-aminium chloride;
(S)-6-(((1-(2-(3-Fluoro-6-methoxy-1,5-naphthyridin-4-yl)-1-hydroxyethyl)-2-oxabicyclo[2.2.2]octan-4-yl)ammonio)methyl)-3,4-dihydro-2H-pyrido[3,2-b][1,4]oxazin-4-ium chloride;
6-(((1-(2-(3-Fluoro-6-methoxy-1,5-naphthyridin-4-yl)ethyl)-2-oxabicyclo[2.2.2]octan-4-yl)ammonio)methyl)-2,3-dihydro-1H-pyrido[2,3-b][1,4]oxazin-1-ium chloride;
6-(((1-(2-(3-Fluoro-6-methoxy-1,5-naphthyridin-4-yl)-2-hydroxyethyl)-2-thiabicyclo[2.2.2]octan-4-yl)amino)methyl)-2H-pyrido[3,2-b][1,4]oxazin-3(4H)-one;
1-(2-(3-Fluoro-6-(2-hydroxyethoxy)-1,5-naphthyridin-4-yl)-2-hydroxypropyl)-N-((3-oxo-3,4-dihydro-2H-pyrido[3,2-b][1,4]oxazin-6-yl)methyl)-2-oxabicyclo[2.2.2]octan-4-aminium chloride;
1-(2-(3-Fluoro-6-(3-hydroxypropoxy)-1,5-naphthyridin-4-yl)-2-hydroxyethyl)-N-((3-oxo-3,4-dihydro-2H-pyrido[3,2-b][1,4]oxazin-6-yl)methyl)-2-oxabicyclo[2.2.2]octan-4-aminium chloride;
4-(2-Hydroxy-2-(4-((3-oxo-3,4-dihydro-2H-pyrido[3,2-b][1,4]oxazin-6-yl)methylamino)-2-oxabicyclo[2.2.2]octan-1-yl)ethyl)-6-methoxy-1,5-naphthyridine-3-carbonitrile Hydrochloride;
6-(2-Hydroxyethoxy)-4-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-3-carbonitrile;
6-(3-Hydroxypropoxy)-4-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-3-carbonitrile;
6-((1-(2-(6-(((1S,3R,4S)-3,4-Dihydroxycyclopentyl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
8-(2-(4-((3-Oxo-3,4-dihydro-2H-pyrido[3,2-b][1,4]oxazin-6-yl)methylamino)-2-oxabicyclo[2.2.2]octan-1-yl)ethyl)-1,5-naphthyridine-2,7-dicarbonitrile;
6-((1-(1-Hydroxy-2-(7-methoxy-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one Hydrochloride;
6-((1-(2-(6-((1-Aminocyclopropyl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(7-Methoxy-4-oxoquinolin-1(4H)-yl)ethyl)-2-oxabicyclo[2.2.2]octan-4-ylamino)methyl)-2H-pyrido[3,2-b][1,4]oxazin-3(4H)-one;
1-(2-(4-((2,3-Dihydro-[1,4]dioxino[2,3-c]pyridin-7-yl)methylamino)-2-oxabicyclo[2.2.2]octan-1-yl)-2-hydroxyethyl)-7-methoxy-1,5-naphthyridin-2(1H)-one;
4-(2-(4-((2,3-Dihydro-[1,4]dioxino[2,3-c]pyridin-7-yl)methylamino)-2-oxabicyclo[2.2.2]octan-1-yl)-2-hydroxyethyl)-6-methoxypyrido[3,2-b]pyrazin-3(4H)-one;
6-((1-(2-(6-((3R,4S)-4-Aminotetrahydrofuran-3-yloxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(6-((3S,4R)-4-Aminotetrahydrofuran-3-yloxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-(3-hydroxypropoxy)-1,5-naphthyridin-4-yl)-1-hydroxyethyl)-2-oxabicyclo[2.2.2]octan-4-ylamino)methyl)-2H-pyrido[3,2-b][1,4]oxazin-3(4H)-one Hydrochloride;
5-(2-(4-((2,3-Dihydro-[1,4]dioxino[2,3-c]pyridin-7-yl)methylamino)-2-oxabicyclo[2.2.2]octan-1-yl)-2-hydroxyethyl)-6-oxo-5,6-dihydro-1,5-naphthyridine-3-carbonitrile;
5-(2-Hydroxy-2-(4-((3-oxo-3,4-dihydro-2H-pyrido[3,2-b][1,4]oxazin-6-yl)methylamino)-2-oxabicyclo[2.2.2]octan-1-yl)ethyl)-6-oxo-5,6-dihydro-1,5-naphthyridine-3-carbonitrile;
5-(2-(4-((2,3-Dihydro-[1,4]dioxino[2,3-c]pyridin-7-yl)methylamino)-2-oxabicyclo[2.2.2]octan-1-yl)-2-hydroxyethyl)-6-oxo-5,6-dihydro-1,5-naphthyridine-3-carbonitrile;
5-(2-Hydroxy-2-(4-((3-oxo-3,4-dihydro-2H-pyrido[3,2-b][1,4]oxazin-6-yl)methylamino)-2-oxabicyclo[2.2.2]octan-1-yl)ethyl)-6-oxo-5,6-dihydro-1,5-naphthyridine-3-carbonitrile;
7-((1-(2-(3-Fluoro-6-methoxy-1,5-naphthyridin-4-yl)ethyl)-2-oxabicyclo[2.2.2]octan-4-ylamino)methyl)-1H-pyrido[2,3-e][1,3,4]oxathiazine-2,2-dioxide;
6-((1-(2-(6-(((2S,3R)-3-Amino-4-oxoazetidin-2-yl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(6-(((1r,3R,4S)-3,4-Dihydroxycyclopentyl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-((3-hydroxyoxetan-3-yl)methoxy)-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-(2-hydroxyethoxy)-1,5-naphthyridin-4-yl)-1-hydroxyethyl)-2-oxabicyclo[2.2.2]octan-4-ylamino)methyl)-2H-pyrido[3,2-b][1,4]oxazin-3(4H)-one Hydrochloride;
3-((1-(2-(3-Fluoro-6-methoxy-1,5-naphthyridin-4-yl)ethyl)-2-oxabicyclo[2.2.2]octan-4-ylamino)methyl)-1-methyl-1,8-naphthyridin-2(1H)-one Hydrochloride;
6-((1-(2-(3-Fluoro-6-((5-hydroxy-4-oxo-1,4-dihydropyridin-2-yl)methoxy)-1,5-naphthyridin-4-yl)ethyl)-2-oxabicyclo[2.2.2]octan-4-ylamino)methyl)-2H-pyrido[3,2-b][1,4]oxazin-3(4H)-one;
Methyl 2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylate;
6-((1-(2-(3-Fluoro-6-methoxy-1,5-naphthyridin-4-yl)-2-hydroxyethyl)-2-oxabicyclo[2.2.2]octan-4-ylamino)methyl)-2H-pyrido[3,2-b][1,4]oxazin-3(4H)-one;
3-((1-(2-(3-Fluoro-6-methoxy-1,5-naphthyridin-4-yl)ethyl)-2-oxabicyclo[2.2.2]octan-4-ylamino)methyl)-1-methyl-1,5-naphthyridin-2(1H)-one;
2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylic Acid;
6-((1-(2-(3-Fluoro-6-hydroxy-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-(2-(5-hydroxy-4-oxo-1,4-dihydropyridin-2-yl)ethoxy)-1,5-naphthyridin-4-yl)ethyl)-2-oxabicyclo[2.2.2]octan-4-ylamino)methyl)-2H-pyrido[3,2-b][1,4]oxazin-3(4H)-one;
7-((1-(2-(3-Fluoro-6-methoxy-1,5-naphthyridin-4-yl)ethyl)-2-oxabicyclo[2.2.2]octan-4-ylamino)methyl)-1H-pyrido[2,3-b][1,4]oxazin-2(3H)-one Hydrochloride;
6-((1-(2-(6-((3R,4S)-4-Aminotetrahydrofuran-3-yloxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
8-Chloro-3-((1-(2-(3-fluoro-6-methoxy-1,5-naphthyridin-4-yl)ethyl)-2-oxabicyclo[2.2.2]octan-4-ylamino)methyl)-1-methylquinolin-2(1H)-one;
(E)-6-Methoxy-4-(2-(4-((3-oxo-3,4-dihydro-2H-pyrido[3,2-b][1,4]oxazin-6-yl)methylamino)-2-oxabicyclo[2.2.2]octan-1-yl)vinyl)pyrido[3,2-c]pyridazine-3-carbonitrile;
6-((1-(1-Hydroxy-2-(7-(2-hydroxyethoxy)-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one;
6-Methoxy-4-(2-(4-((3-oxo-3,4-dihydro-2H-pyrido[3,2-b][1,4]oxazin-6-yl)methylamino)-2-oxabicyclo[2.2.2]octan-1-yl)ethyl)pyrido[3,2-c]pyridazine-3-carbonitrile;
6-((1-(1-Hydroxy-2-(7-(3-hydroxypropoxy)-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one;
6-((1-(2-(6-((3S,4S)-4-Amino-5-oxopyrrolidin-3-yloxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
methyl 2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylate;
2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylic Acid;
6-((1-(2-(6-(((2R,3R)-3-Aminooxetan-2-yl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
Ethyl 4-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanoate;
4-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanoic Acid;
6-((1-(2-(6-(((2S,3R)-3-Aminooxetan-2-yl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
7-((1-(2-(3-Fluoro-6-methoxy-1,5-naphthyridin-4-yl)ethyl)-2-oxabicyclo[2.2.2]octan-4-ylamino)methyl)-5-methylpyrido[3,2-b]pyrazin-6(5H)-one Hydrochloride;
6-((1-(2-(6-Methoxypyrido[3,2-d]pyrimidin-4-yl)ethyl)-2-oxabicyclo[2.2.2]octan-4-ylamino)methyl)-2H-pyrido[3,2-b][1,4]oxazin-3(4H)-one Hydrochloride;
6-((1-(2-(6-((2-Aminocyclopropyl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
4-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanenitrile;
ethyl 4-(7-Cyano-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanoate;
4-(7-Cyano-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanoic Acid;
6-((1-(2-(6-(((2S,3S)-3-Aminooxetan-2-yl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
4-(2-Hydroxy-2-(4-((3-oxo-3,4-dihydro-2H-pyrido[3,2-b][1,4]oxazin-6-yl)methylamino)-2-oxabicyclo[2.2.2]octan-1-yl)ethyl)-6-oxo-5,6,7,8-tetrahydro-1,5-naphthyridine-3-carbonitrile;
6-((1-(2-(7-(2-Hydroxyethoxy)-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one;
6-((1-(2-(7-(3-Hydroxypropoxy)-2-oxo-1,5-naphthyridin-1(2H)-yl)ethyl)-2-oxabicyclo[2.2.2]octan-4-ylamino)methyl)-2H-pyrido[3,2-b][1,4]oxazin-3(4H)-one;
methyl 5-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)pentanoate;
2-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylic Acid Hydrochloride;
5-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)pentanoic Acid;
methyl 7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-2-carboxylate;
7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridine-2-carboxylic Acid;
6-((1-(2-(6-(((2R,3S)-3-Aminooxetan-2-yl)methoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
methyl 1-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylate;
1-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarboxylic Acid;
methyl 2-((6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridin-3-yloxy)methyl)cyclopropanecarboxylate;
methyl 2-((6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridin-3-yloxy)methyl)cyclopropanecarboxylate;
4-(2-Hydroxy-2-(4-((3-oxo-3,4-dihydro-2H-pyrido[3,2-b][1,4]oxazin-6-yl)methylamino)-2-oxabicyclo[2.2.2]octan-1-yl)ethyl)-6-(2-hydroxyethoxy)-1,5-naphthyridine-3-carbonitrile;
4-(2-Hydroxy-2-(4-((3-oxo-3,4-dihydro-2H-pyrido[3,2-b][1,4]oxazin-6-yl)methylamino)-2-oxabicyclo[2.2.2]octan-1-yl)ethyl)-6-(3-hydroxypropoxy)-1,5-naphthyridine-3-carbonitrile;
methyl 2-((6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridin-3-yloxy)methyl)cyclopropanecarboxylate;
ethyl 2,2-Difluoro-4-(7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanoate;
2,2-Difluoro-4-(7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanamide;
methyl 2-((6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridin-3-yloxy)methyl)cyclopropanecarboxylate;
2,2-Difluoro-4-(7-fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)butanoic Acid;
1-((7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)methyl)cyclopropanecarbonitrile;
2-((6-Oxo-5-(2-(4-((3-oxo-3,4-dihydro-2H-pyrido[3,2-b][1,4]oxazin-6-yl)methylamino)-2-oxabicyclo[2.2.2]octan-1-yl)ethyl)-5,6-dihydro-1,5-naphthyridin-3-yloxy)methyl)cyclopropanecarboxylic Acid Hydrochloride;
6-((1-(2-(6-(Difluoromethoxy)-3-fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
5,8-Difluoro-3-((1-(2-(3-fluoro-6-methoxy-1,5-naphthyridin-4-yl)ethyl)-2-oxabicyclo[2.2.2]octan-4-ylamino)methyl)-1-methylquinolin-2(1H)-one;
6-((1-(2-(3-Fluoro-1,5-naphthyridin-4-yl)ethyl)-2-oxabicyclo[2.2.2]octan-4-ylamino)methyl)-2H-pyrido[3,2-b][1,4]oxazin-3(4H)-one;
2-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)-N-methylacetamide;
N-((5,8-Difluoro-2-methoxyquinolin-3-yl)methyl)-1-(2-(3-fluoro-6-methoxy-1,5-naphthyridin-4-yl)ethyl)-2-oxabicyclo[2.2.2]octan-4-amine;
N-(2-(2,5-Difluorophenoxy)ethyl)-1-(2-(3-fluoro-6-methoxy-1,5-naphthyridin-4-yl)ethyl)-2-oxabicyclo[2.2.2]octan-4-amine;
4-(7-Fluoro-8-((2-(4-((3-oxo-3,4-dihydro-2H-pyrido[3,2-b][1,4]oxazin-6-yl)methyl)amino)-2-oxabicyclo[2.2.2]octan-1-yl)ethyl)-1,5-naphthyridin-2-yl)thiomorpholine-1,1-dioxide;
2-(7-Fluoro-8-(2-(4-((3-oxo-3,4-dihydro-2H-pyrido[3,2-b][1,4]oxazin-6-yl)methylamino)-2-oxabicyclo[2.2.2]octan-1-yl)ethyl)-1,5-naphthyridin-2-yloxy)-N,N-dimethylacetamide;
6-((1-(2-(3-Fluoro-6-methyl-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-(2-oxooxazolidin-3-yl)-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-(4-hydroxypiperidin-1-yl)-1,5-naphthyridin-4-yl)ethyl)-2-oxabicyclo[2.2.2]octan-4-ylamino)methyl)-2H-pyrido[3,2-b][1,4]oxazin-3(4H)-one;
(S)-6-((1-(2-(3-Fluoro-6-(3-hydroxypyrrolidin-1-yl)-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-(3-hydroxyazetidin-1-yl)-1,5-naphthyridin-4-yl)ethyl)-2-oxabicyclo[2.2.2]octan-4-ylamino)methyl)-2H-pyrido[3,2-b][1,4]oxazin-3(4H)-one;
(R)-6-((1-(2-(3-Fluoro-6-(3-hydroxypyrrolidin-1-yl)-1,5-naphthyridin-4-yl)ethyl)-2-oxabicyclo[2.2.2]octan-4-ylamino)methyl)-2H-pyrido[3,2-b][1,4]oxazin-3(4H)-one;
6-((1-(2-(3-Fluoro-6-(2-hydroxyethylamino)-1,5-naphthyridin-4-yl)ethyl)-2-oxabicyclo[2.2.2]octan-4-ylamino)methyl)-2H-pyrido[3,2-b][1,4]oxazin-3(4H)-one;
2-(8-(2-(4-((2,3-Dihydro-[1,4]dioxino[2,3-c]pyridin-7-yl)methylamino)-2-oxabicyclo[2.2.2]octan-1-yl)ethyl)-7-fluoro-1,5-naphthyridin-2-yloxy)-N-methylacetamide;
(E)-2-(8-(2-(4-(3-(2,5-Difluorophenyl)allylamino)-2-oxabicyclo[2.2.2]octan-1-yl)ethyl)-7-fluoro-1,5-naphthyridin-2-yloxy)-N-methylacetamide;
(E)-1-(2-(3-Fluoro-6-methoxy-1,5-naphthyridin-4-yl)ethyl)-N-(3-(pyridin-2-yl)allyl)-2-oxabicyclo[2.2.2]octan-4-amine;
or a pharmaceutically acceptable salt or stereoisomer thereof.

15. A composition comprising the compound of claim 1 and a pharmaceutically acceptable carrier, adjuvant or vehicle.

16. The composition according to claim 15, further comprising a second therapeutic agent is selected from the group consisting of carbapenems, penicillins, and cephalosporins.

17. A method of treating a bacterial infection in a patient in need thereof, comprising administering to said patient an effective amount of the compound of claim 1.

18. The method of claim 17 further comprising administration of an effective amount of a second therapeutic agent.

19. (canceled)
